(19) United States
(12) Patent Application Publication
(10) Pub. No.: US 2024/0409617 A1
(43) Pub. Date: Dec. 12, 2024

(54) MULTISPECIFIC CORONAVIRUS ANTIBODIES

(71) Applicant: Dana-Farber Cancer Institute, Inc., Boston, MA (US)

(72) Inventors: Wayne A. Marasco, Wellesley, MA (US); Matthew Chang, Brookline, MA (US)

(21) Appl. No.: 18/014,236

(22) PCT Filed: Jul. 6, 2021
(86) PCT No.: PCT/US2021/040418

§ 371 Date: Jan. 3, 2023

Related U.S. Application Data
(60) Provisional application No. 63/048,046, filed on Jul. 3, 2020.
(60) Provisional application No. 63/069,461, filed on Aug. 24, 2020.

(51) Int. Cl. C07K 16/10; A61P 31/14

(52) U.S. Cl. CPC C07K 16/1003; A61P 31/14; C07K 2317/21; C07K 2317/622; C07K 2317/76

(57) ABSTRACT

This invention provides multispecific antibodies that neutralize SARS-CoV2 and methods of use thereof. The antibodies described herein can be used to treat SARS-CoV2 infections and symptoms thereof.

This application is an International Application which claims priority from U.S. Provisional Patent Application Nos. 63/048,046 filed on Jul. 3, 2020, and 63/069,461 filed on Aug. 24, 2020, the contents of which is incorporated herein by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

This application contains a Sequence Listing which has been submitted electronically in ASCII format and is hereby incorporated by reference in its entirety. Said ASCII copy, created on [ ] is named [ ] and is [ ] bytes in size.

### FIELD OF THE INVENTION

Aspects of this invention are drawn to severe acute respiratory syndrome-associated coronavirus 2 (SARS-CoV2) neutralizing antibodies as well as to methods for use thereof.

### BACKGROUND

Human monoclonal antibody (mAb) therapy offers considerable advantages for prophylaxis, preemptive and acute treatment in viral outbreak settings.

### SUMMARY

An aspect of the invention is directed to isolated monoclonal antibodies directed to Severe Acute Respiratory Syndrome coronavirus (SARS-CoV2). In some embodiments, the antibody binds to an epitope in SEQ ID NO: 979. In some embodiments, the antibody binds to an epitope in the receptor binding domain (RBD) of the spike protein (S). In some embodiments, the antibody neutralizes SARS-CoV2. In some embodiments the epitope is linear. In other embodiments, the epitope is non-linear. In some embodiments, the epitope comprises a region within amino acids 319-490 of SEQ ID NO: 980 of the spike protein. In other embodiments, the epitope comprises a region within amino acids 319-541 SEQ ID NO: 980 of the spike protein. In further embodiments, the monoclonal antibody inhibits viral and cell membrane fusion. In yet other embodiments, the monoclonal antibody competes with the binding of a monoclonal antibody to the spike protein. In some embodiments, the monoclonal antibody blocks the binding of SARS-CoV2 spike protein to angiotensin converting enzyme 2 (ACE2) cell surface receptor. In another embodiment, the monoclonal antibody is a fully human antibody. In some embodiments, the monoclonal antibody comprises: (a) a heavy chain with three CDRs comprising the amino acid sequences GGSIRTHS (SEQ ID NO:93), IHHSGAT (SEQ ID NO:94), and ARGPGILSY (SEQ ID NO:95) respectively, and/or a light chain with three CDRs comprising the amino acid sequences SSNIGSNT (SEQ ID NO:227), SNN (SEQ ID NO:228), and AAWDDSLNVHYV (SEQ ID NO:229) respectively; (b) a heavy chain with three CDRs comprising the amino acid sequences GGSISSYY (SEQ ID NO:96), IYTSGST (SEQ ID NO:97), and ARDVGFGWFDR (SEQ ID NO:98) respectively and/or a light chain with three CDRs comprising the amino acid sequences SGSIASNY (SEQ ID NO:230), EDN (SEQ ID NO:231), and QSFDSASLWV (SEQ ID NO:232) respectively; (c) a heavy chain with three CDRs comprising the amino acid sequences GGSIRTHS (SEQ ID NO:99), IHHSGAT (SEQ ID NO:100), and ARGPGILSY (SEQ ID NO:101) respectively and/or a light chain with three CDRs comprising the amino acid sequences SSNIGSND (SEQ ID NO:233), SNN (SEQ ID NO:234), and ATWDDSLSAGV (SEQ ID NO:235) respectively; (d) a heavy chain with three CDRs comprising the amino acid sequences GDSVSSYSDA (SEQ ID NO:102), TYYRSKWYN (SEQ ID NO:103), and AREIVATTPFRNYYYGMDV (SEQ ID NO:104) respectively and/or a light chain with three CDRs comprising the amino acid sequences SGSIASNY (SEQ ID NO:236), QDK (SEQ ID NO:237), and QSYDSSSLWV (SEQ ID NO:238) respectively; (e) a heavy chain with three CDRs comprising the amino acid sequences GFTFSHYD (SEQ ID NO:105), IGYDGTNL (SEQ ID NO: 106), and ARAANYYDSSGYGRADAFDI (SEQ ID NO:107) respectively and/or a light chain with three CDRs comprising the amino acid sequences TGSIAGNY (SEQ ID NO:239), DDN (SEQ ID NO:240), and QSYDSGNRGV (SEQ ID NO:241) respectively; (f) a heavy chain with three CDRs comprising the amino acid sequences GFTFSDFP (SEQ ID NO:108), ISYDGNIK (SEQ ID NO:109), and ARGGSSFDI (SEQ ID NO:110) respectively and/or a light chain with three CDRs comprising the amino acid sequences TSNIGNNA (SEQ ID NO:242), YNE (SEQ ID NO:243), and AAWDDSLSGHVV (SEQ ID NO:244) respectively; (g) a heavy chain with three CDRs comprising the amino acid sequences GFSLSTTGVG (SEQ ID NO:111), IYWNDDK (SEQ ID NO:112), and ARISGSGYFYPFDI (SEQ ID NO:113) respectively and/or a light chain with three CDRs comprising the amino acid sequences SGSIASNY (SEQ ID NO:245), EDN (SEQ ID NO:246), and QSYDSSSLWV (SEQ ID NO:247) respectively; (h) a heavy chain with three CDRs comprising the amino acid sequences GYTFSDYY (SEQ ID NO: 120), IDPNSGGT (SEQ ID NO:121), and ARDRGRGGQAGAFDY (SEQ ID NO:978) respectively and/or a light chain with three CDRs comprising the amino acid sequences KIGSKS (SEQ ID NO:254), DDS (SEQ ID NO:255), and HVWDSSSDQNV (SEQ ID NO:256) respectively; (i) a heavy chain with three CDRs comprising the amino acid sequences GFTFSSYA (SEQ ID NO:122), ISYGGSNK (SEQ ID NO:123), and AKVRGSGWYWGSAFDI (SEQ ID NO:124) respectively and/or a light chain with three CDRs comprising the amino acid sequences SLRAYF (SEQ ID NO:257), GQD (SEQ ID NO:258), and NSRDSGENHLI (SEQ ID NO:259) respectively; (j) a heavy chain with three CDRs comprising the amino acid sequences GYSFTGSH (SEQ ID NO:125), INPDSGVI (SEQ ID NO:126), and ARDKAIGYVWALDY (SEQ ID NO:127) respectively and/or a light chain with three CDRs comprising the amino acid sequences SSDVGTYNR (SEQ ID NO:260), EVS (SEQ ID NO:261), and SSYTRTFTYV (SEQ ID NO:262) respectively; (k) a heavy chain with three CDRs comprising the amino acid sequences GVSLDTIGMR (SEQ ID NO:128), IDWDDDK (SEQ ID NO: 129), and ARSGLLYDLDV (SEQ ID NO:130) respectively and/or a light chain with three CDRs comprising the amino acid sequences DSDIGANF (SEQ ID NO:263), RNT (SEQ ID NO:264), and QSYDSSLSAYV (SEQ ID NO:265) respectively; (l) a heavy chain with three CDRs comprising the amino acid sequences GYSFTSYW (SEQ ID NO:134), IYPGDSDT (SEQ ID NO:135), and ARGWQWHDY (SEQ ID NO:136) respectively and/or a light chain with three CDRs comprising the amino acid sequences SLRSYY (SEQ ID NO:269), DKD (SEQ ID NO:270), and NSRDRSDNHVV (SEQ ID NO:271) respectively; (m) a heavy chain with three CDRs comprising the amino acid sequences GDSVSSRSSA (SEQ ID NO:137), TYYRSNWNY (SEQ ID NO:138), and VRNMRPDFDL (SEQ ID NO:139) respectively and/or a light chain with three CDRs comprising the amino acid sequences QSVSNN (SEQ ID NO:272), DAT (SEQ ID NO:273), and QQYDNLPV (SEQ ID NO:274) respectively; (n) a heavy chain with three CDRs comprising the amino acid sequences GYTFTTSG (SEQ ID NO:140), ISAYNGNT (SEQ ID NO:141), and ARDFHLYYGMDV (SEQ ID NO:142) respectively and/or a light chain with three CDRs comprising the amino acid sequences SSDVGAYNY (SEQ ID NO:275), DVT (SEQ ID NO:276), and AVWDDGLNGRVV (SEQ ID NO:277) respectively; (o) a heavy chain with three CDRs comprising the amino acid sequences GGTFSSYA (SEQ ID NO:143), INPNSGGT (SEQ ID NO:144), and ARGSGGYYLG (SEQ ID NO:145) respectively and/or a light chain with three CDRs comprising the amino acid sequences SNNVGNQG (SEQ ID NO:278), MNN (SEQ ID NO:279), and SAWDSSLSRWV (SEQ ID NO:280) respectively; (p) a heavy chain with three CDRs comprising the amino acid sequences GGTFSSYT (SEQ ID NO: 146), IIPILGTP (SEQ ID NO: 147), and AVGSGWYSGFDY (SEQ ID NO:148) respectively and/or a light chain with three CDRs comprising the amino acid sequences SGSIASNY (SEQ ID NO:281), EDS (SEQ ID NO:282), and QSFHNSNPVI (SEQ ID NO:283) respectively; (q) a heavy chain with three CDRs comprising the amino acid sequences GFTFSSYW (SEQ ID NO:149), IKQDGSEK (SEQ ID NO:150), and ARGFYYYGAFDI (SEQ ID NO:151) respectively and/or a light chain with three CDRs comprising the amino acid sequences SGSIASNY (SEQ ID NO:284), EDN (SEQ ID NO:285), and QSYDSSNHWV (SEQ ID NO:286) respectively; (r) a heavy chain with three CDRs comprising the amino acid sequences GFTFDDYA (SEQ ID NO:152), IDWNSGVI (SEQ ID NO:153), and AKDAYSYGFLGAFDI (SEQ ID NO:154) respectively and/or a light chain with three CDRs comprising the amino acid sequences NIGSKS (SEQ ID NO:287), EDR (SEQ ID NO:288), and QVWDGDSDHYV (SEQ ID NO:289) respectively; (s) a heavy chain with three CDRs comprising the amino acid sequences GFTFDDYA (SEQ ID NO:155), IDWNSGVI (SEQ ID NO:156), and ARDILPSNFDGKKIIVFQPPAKRDLDNYYGMDV (SEQ ID NO:157) respectively and/or a light chain with three CDRs comprising the amino acid sequences SSDVGGYNL (SEQ ID NO:290), EGS (SEQ ID NO:291), and SSYTITDVVV (SEQ ID NO:292) respectively; or (t) a heavy chain with three CDRs comprising the amino acid sequences GYSFTSNW (SEQ ID NO:158), IFPGDSDT (SEQ ID NO:159), and ARESYNAYGS (SEQ ID NO:160) respectively and/or a light chain with three CDRs comprising the amino acid sequences SSNIGSNP (SEQ ID NO:293), SNN (SEQ ID NO:294), and AAWDDSLSGVV (SEQ ID NO:295) respectively.

In other embodiments, the monoclonal antibody comprises: (a) a heavy chain with three CDRs comprising the amino acid sequences GFTFTTYG (SEQ ID NO:114), ISYDGSIK (SEQ ID NO:115), and ARVGDSSSYYGIDA (SEQ ID NO: 116) respectively, and/or a light chain with three CDRs comprising the amino acid sequences SSNIGSNS (SEQ ID NO:248), SNN (SEQ ID NO:249), and AAWDDSLTGYV (SEQ ID NO:250) respectively; (b) a heavy chain with three CDRs comprising the amino acid sequences GFTFSSHA (SEQ ID NO:117), ISYDGSYT (SEQ ID NO:118), and ARDWVNFGMDV (SEQ ID NO:119) respectively, and/or a light chain with three CDRs comprising the amino acid sequences SSDVGGYNY (SEQ ID NO:251), EVS (SEQ ID NO:252), and AAWDDSLSGPV (SEQ ID NO:253) respectively; or (c) a heavy chain with three CDRs comprising the amino acid sequences GFTFSDYP (SEQ ID NO:131), TSYDGRIK (SEQ ID NO:132), and ARDPGWLRSVGMDV (SEQ ID NO:133) respectively, and/or a light chain with three CDRs comprising the amino acid sequences SGSIARNY (SEQ ID NO:266), ADR (SEQ ID NO:267), and QSYDSSNQAAV (SEQ ID NO:268) respectively. In yet further embodiments, the monoclonal antibody comprises:
a) a heavy chain with three CDRs comprising the amino acid sequences GYTFTSYG (SEQ ID NO:161), ISAYNGNT (SEQ ID NO:162), and ARGFPQLGSDY (SEQ ID NO: 163) respectively, and/or a light chain with three CDRs comprising the amino acid sequences SGSIASNY (SEQ ID NO:296), EDN (SEQ ID NO:297), and QSYDSTNWV (SEQ ID NO:298) respectively;
b) a heavy chain with three CDRs comprising the amino acid sequences GGTFSSYA (SEQ ID NO:164), ISGYNGNT (SEQ ID NO:165), and ARQMKDSGNYWEYYYYGMDV (SEQ ID NO:166) respectively, and/or a light chain with three CDRs comprising the amino acid sequences NIGSES (SEQ ID NO:299), EDR (SEQ ID NO:300), and QVWNPSGSLQYV (SEQ ID NO:301) respectively;
c) a heavy chain with three CDRs comprising the amino acid sequences GYTFTSYG (SEQ ID NO:167), ISTYNGNT (SEQ ID NO:168), and ARDVFGHFDY (SEQ ID NO:169) respectively, and/or a light chain with three CDRs comprising the amino acid sequences SGNIATNY (SEQ ID NO:302), EDN (SEQ ID NO:303), and KSYDDGNHV (SEQ ID NO:304) respectively;
d) a heavy chain with three CDRs comprising the amino acid sequences GFSLTTTGVS (SEQ ID NO:170), IHWDDDK (SEQ ID NO:171), and ASFIMTVYAEYFED (SEQ ID NO: 172) respectively, and/or a light chain with three CDRs comprising the amino acid sequences QSVSSN (SEQ ID NO:305), DVS (SEQ ID NO:306), and QQRGVWPLT (SEQ ID NO:307) respectively;
e) a heavy chain with three CDRs comprising the amino acid sequences GFSLSTSAMC (SEQ ID NO:173), IDWDNDR (SEQ ID NO:174), and AHSPYDSIWGSFRPSVYYFDY (SEQ ID NO:175) respectively, and/or a light chain with three CDRs comprising the amino acid sequences SGSIVSSY (SEQ ID NO:308), EHN (SEQ ID NO:309), and QSYDSQNGV (SEQ ID NO:310) respectively;
f) a heavy chain with three CDRs comprising the amino acid sequences GFTFSDYY (SEQ ID NO:176), ISSSSSDT (SEQ ID NO:177), and AMPTREPAY (SEQ ID NO:178) respectively, and/or a light chain with three CDRs comprising the amino acid sequences SSDLGTYNY (SEQ ID NO:311), DVF (SEQ ID NO:312), and SSYTSSSTYV (SEQ ID NO:313) respectively;
g) a heavy chain with three CDRs comprising the amino acid sequences GFAFSDFP (SEQ ID NO: 179), ISYDGSLK (SEQ ID NO:180), and AREGVSNSRPFDH (SEQ ID NO:181) respectively, and/or a light chain with three CDRs comprising the amino acid sequences SIGTKS (SEQ ID NO: 314), DDD (SEQ ID NO:315), and QVWESDDDDLV (SEQ ID NO:316) respectively;
h) a heavy chain with three CDRs comprising the amino acid sequences GFTFSSYA (SEQ ID NO:182), ISSNGGST (SEQ ID NO:183), and TRDLWSGSADSFDI (SEQ ID NO:184) respectively, and/or a light chain with three CDRs comprising the amino acid sequences SLRRYY (SEQ ID NO:317), GKN (SEQ ID NO:318), and NSRDISDNQWQWI (SEQ ID NO:319) respectively;
i) a heavy chain with three CDRs comprising the amino acid sequences GFPFNAYY (SEQ ID NO:185), INQDGSEK (SEQ ID NO:186), and ARLYWWGMDV (SEQ ID NO:187) respectively, and/or a light chain with three CDRs comprising the amino acid sequences SSDVGGYKY (SEQ ID NO:320), DVN (SEQ ID NO:321), and SSYTGRMNLYV (SEQ ID NO:322) respectively;
j) a heavy chain with three CDRs comprising the amino acid sequences GFTFDDYA (SEQ ID NO:188), IDWNSGVI (SEQ ID NO:189), and AKDAYSYGFLGAFDI (SEQ ID NO:190) respectively, and/or a light chain with three CDRs comprising the amino acid sequences NIRTKG (SEQ ID NO:323), YAS (SEQ ID NO:324), and QVWDSSSDLVV (SEQ ID NO:325) respectively;
k) a heavy chain with three CDRs comprising the amino acid sequences GFTFDDYA (SEQ ID NO:191), ISWNSGSI (SEQ ID NO:192), and ARDWWGSIDH (SEQ ID NO:193) respectively, and/or a light chain with three CDRs comprising the amino acid sequences SSDVGGYDY (SEQ ID NO:326), DVS (SEQ ID NO:327), and SSYTSSSPVV (SEQ ID NO:328) respectively;
l) a heavy chain with three CDRs comprising the amino acid sequences GGSISSSNW (SEQ ID NO:194), IYHSGST (SEQ ID NO:195), and ARRGGTYHRGAFDI (SEQ ID NO:196) respectively, and/or a light chain with three CDRs comprising the amino acid sequences SRDVGSYDL (SEQ ID NO:329), EGS (SEQ ID NO:330), and SSYTSSNSLV (SEQ ID NO:331) respectively;
m) a heavy chain with three CDRs comprising the amino acid sequences GASISNSF (SEQ ID NO:197), TSYSGNS (SEQ ID NO:198), and ARREWIKGHFDY (SEQ ID NO:199) respectively, and/or a light chain with three CDRs comprising the amino acid sequences GGSIASNY (SEQ ID NO:332), EDN (SEQ ID NO:333), and QSYDSSNPVV (SEQ ID NO:334) respectively;
n) a heavy chain with three CDRs comprising the amino acid sequences GGSFTTHS (SEQ ID NO:200), ILPGGAT (SEQ ID NO:201), and ARGPGILSY (SEQ ID NO:202) respectively, and/or a light chain with three CDRs comprising the amino acid sequences SSIGSND (SEQ ID NO:335), SNN (SEQ ID NO:336), and AWDDSLSAVV (SEQ ID NO:337) respectively;
o) a heavy chain with three CDRs comprising the amino acid sequences GGSFRTHS (SEQ ID NO:203), IHHSGAT (SEQ ID NO:204), and ARGPGILSY (SEQ ID NO:205) respectively, and/or a light chain with three CDRs comprising the amino acid sequences SSNIGSNT (SEQ ID NO:338), INN (SEQ ID NO:339), and AEWYDSLNVHYV (SEQ ID NO:340) respectively;
p) a heavy chain with three CDRs comprising the amino acid sequences GGSIRTHS (SEQ ID NO:206), IHHSGAT (SEQ ID NO:207), and ARGPGILSY (SEQ ID NO:208) respectively, and/or a light chain with three CDRs comprising the amino acid sequences SSNIGSNT (SEQ ID NO:341), INN (SEQ ID NO:342), and AECYDSLNDHYV (SEQ ID NO:343) respectively;
q) a heavy chain with three CDRs comprising the amino acid sequences GGSIRTHS (SEQ ID NO:209), IHHSGAT (SEQ ID NO:210), and GRGPGILSY (SEQ ID NO:211) respectively, and/or a light chain with three CDRs comprising the amino acid sequences SSNIGSNT (SEQ ID NO:344), SNN (SEQ ID NO:345), and AAWDDSLNVHYV (SEQ ID NO:346) respectively;
r) a heavy chain with three CDRs comprising the amino acid sequences GYSFTSYW (SEQ ID NO:212), IYPGDSDT (SEQ ID NO:213), and ARQGDGGGYDY (SEQ ID NO:214) respectively, and/or a light chain with three CDRs comprising the amino acid sequences SSNIGSNP (SEQ ID NO:347), NNN (SEQ ID NO:348), and AAWDDSLNGL (SEQ ID NO:349) respectively;
s) a heavy chain with three CDRs comprising the amino acid sequences RYSFSNYW (SEQ ID NO:215), IYPYDSDT (SEQ ID NO:216), and ARQGSSQSFDI (SEQ ID NO:217) respectively, and/or a light chain with three CDRs comprising the amino acid sequences SLRSYY (SEQ ID NO:350), GKN (SEQ ID NO:351), and NSRDSSGDVRV (SEQ ID NO:352) respectively;
t) a heavy chain with three CDRs comprising the amino acid sequences GYSFTSYW (SEQ ID NO:218), IYPGDSDT (SEQ ID NO:219), and ARRRGSAAAFDT (SEQ ID NO:220) respectively, and/or a light chain with three CDRs comprising the amino acid sequences SSNIGSNP (SEQ ID NO:353), DNN (SEQ ID NO:354), and EAWDDSLSGPV (SEQ ID NO:355) respectively;
u) a heavy chain with three CDRs comprising the amino acid sequences GYSFTSYW (SEQ ID NO:221), IYPGDSDT (SEQ ID NO:222), and ARTTYSYGSFDY (SEQ ID NO:223) respectively, and/or a light chain with three CDRs comprising the amino acid sequences SSNIGGNS (SEQ ID NO:356), RNN (SEQ ID NO:357), and AAWDDSLNGWV (SEQ ID NO:358) respectively; or
v) a heavy chain with three CDRs comprising the amino acid sequences GDSVTSNSAA (SEQ ID NO:224), TYYSSKWYN (SEQ ID NO:225), and ARGWLRLSFDP (SEQ ID NO:226) respectively, and/or a light chain with three CDRs comprising the amino acid sequences SGSIASNY (SEQ ID NO:359), EDN (SEQ ID NO:360), and QSYDPNNHGVV (SEQ ID NO:361) respectively.

In other embodiments, the monoclonal antibody comprises: a) a heavy chain with three CDRs comprising the amino acid sequences GFSLSTSGVG (SEQ ID NO:754), IYWDDDK (SEQ ID NO:755), and ARISGSGYFYPFDI (SEQ ID NO:756) respectively, and/or a light chain with three CDRs comprising the amino acid sequences SGSIASNY (SEQ ID NO:802), EDN (SEQ ID NO:803), and QSYDSSNLWV (SEQ ID NO:804) respectively; b) a heavy chain with three CDRs comprising the amino acid sequences GDSVSSNSAA (SEQ ID NO:757), TYYRSRWYN (SEQ ID NO:758), and AREIRGFDY (SEQ ID NO:759) respectively, and/or a light chain with three CDRs comprising the amino acid sequences SSDVGAYNF (SEQ ID NO:805), DFN (SEQ ID NO:806), and SSYAGSNNFDVV (SEQ ID NO:807) respectively; c) a heavy chain with three CDRs comprising the amino acid sequences GFTFGDYA (SEQ ID NO:760), IRSKAYGGTT (SEQ ID NO:761), and TTADDDMDV (SEQ ID NO:762) respectively, and/or a light chain with three CDRs comprising the amino acid sequences SGTIASNY (SEQ ID NO:808), EDN (SEQ ID NO:809), and QSYDTSNHYV (SEQ ID NO:810) respectively; d) a heavy chain with three CDRs comprising the amino acid sequences GFTFSNYG (SEQ ID NO:763), IWERGSKK (SEQ ID NO:764), and AREGISMTGAEYFQH (SEQ ID NO:765) respectively, and/or a light chain with three CDRs comprising the amino acid sequences SSNIGAGYD (SEQ ID NO:811), GTN (SEQ ID NO:812), and QSYDNSLTDPYV (SEQ ID NO:813) respectively; e) a heavy chain with three CDRs comprising the amino acid sequences GFTFDDYA (SEQ ID NO:766), IDWNSGVI (SEQ ID NO:767), and AKDIGPGGSGSYYAFDI (SEQ ID NO:768) respectively, and/or a light chain with three CDRs comprising the amino acid sequences SSDVGGSKY (SEQ ID NO:814), DVT (SEQ ID NO:815), and AAWDDSLNGVV (SEQ ID NO:816) respectively; f) a heavy chain with three CDRs comprising the amino acid sequences GFSFSRYG (SEQ ID NO:769), IRHDGSKK (SEQ ID NO:770), and AKDGRLEAALDD (SEQ ID NO:771) respectively, and/or a light chain with three CDRs comprising the amino acid sequences SGSIANNF (SEQ ID NO:817), EDN (SEQ ID NO:818), and QSYDSSNLV (SEQ ID NO:819) respectively; g) a heavy chain with three CDRs comprising the amino acid sequences GYSFTSYW (SEQ ID NO:772), IYPGDSDT (SEQ ID NO:773), and ARRGDLDAFDI (SEQ ID NO:774) respectively, and/or a light chain with three CDRs comprising the amino acid sequences SANIGSNA (SEQ ID NO:820), GNT (SEQ ID NO:821), and AAWDDSLNGYV (SEQ ID NO:822) respectively; h) a heavy chain with three CDRs comprising the amino acid sequences GYRLSDYY (SEQ ID NO:775), IKQDGSEK (SEQ ID NO:776), and ARVRGWSRGYFDY (SEQ ID NO:777) respectively, and/or a light chain with three CDRs comprising the amino acid sequences SGSIASNY (SEQ ID NO:823), EDN (SEQ ID NO:824), and QSYDSSNHWV (SEQ ID NO:825) respectively; i) a heavy chain with three CDRs comprising the amino acid sequences GFTFDDYA (SEQ ID NO:778), ISWNSGSI (SEQ ID NO:779), and ARDWWGSIDH (SEQ ID NO:780) respectively, and/or a light chain with three CDRs comprising the amino acid sequences SSDVGGYDY (SEQ ID NO:826), DVS (SEQ ID NO:827), and SSYTSSSPVV (SEQ ID NO:828) respectively; j) a heavy chain with three CDRs comprising the amino acid sequences GFTFSHYD (SEQ ID NO:781), IGYDGTNL (SEQ ID NO:782), and ARAANYYDSSGYGRADAFDI (SEQ ID NO:783) respectively, and/or a light chain with three CDRs comprising the amino acid sequences TGSIAGNY (SEQ ID NO:829), DDN (SEQ ID NO:830), and QSYDSGNRGV (SEQ ID NO:831) respectively; k) a heavy chain with three CDRs comprising the amino acid sequences GGTFSTYG (SEQ ID NO:784), IIPSLGIP (SEQ ID NO:785), and ARENIDLATNDF (SEQ ID NO:786) respectively, and/or a light chain with three CDRs comprising the amino acid sequences SRDIGAYGY (SEQ ID NO:832), EVR (SEQ ID NO:833), and SSYTSSSTLDVV (SEQ ID NO:834) respectively; l) a heavy chain with three CDRs comprising the amino acid sequences GGTFSSSG (SEQ ID NO:787), IIPMLGTP (SEQ ID NO:788), and ARDGGNYDY (SEQ ID NO:789) respectively, and/or a light chain with three CDRs comprising the amino acid sequences SSNIGRNA (SEQ ID NO:835), SNN (SEQ ID NO:836), and SAWDTSLSTWV (SEQ ID NO:837) respectively; m) a heavy chain with three CDRs comprising the amino acid sequences GFTFSSYW (SEQ ID NO:790), IKQDGSEK (SEQ ID NO:791), and ARGFYYYGAFDI (SEQ ID NO:792) respectively, and/or a light chain with three CDRs comprising the amino acid sequences SGSIASNY (SEQ ID NO:838), EDN (SEQ ID NO:839), and QSYDSSNHWV (SEQ ID NO:840) respectively; n) a heavy chain with three CDRs comprising the amino acid sequences GFTFDDYA (SEQ ID NO:793), IDWNSGVI (SEQ ID NO:794), and AKDAYSYGFLGAFDI (SEQ ID NO:795) respectively, and/or a light chain with three CDRs comprising the amino acid sequences NIRTKG (SEQ ID NO:841), YAS (SEQ ID NO:842), and QVWDSSSDLVV (SEQ ID NO:843) respectively;
o) a heavy chain with three CDRs comprising the amino acid sequences GYSFTGSH (SEQ ID NO:796), INPDSGVI (SEQ ID NO:797), and ARDKAIGYVWALDY (SEQ ID NO:798) respectively, and/or a light chain with three CDRs comprising the amino acid sequences SSDVGTYNR (SEQ ID NO:844), EVS (SEQ ID NO:845), and SSYTRTFTYV (SEQ ID NO:846) respectively; or p) a heavy chain with three CDRs comprising the amino acid sequences GASISNSF (SEQ ID NO:799), TSYSGNS (SEQ ID NO:800), and ARREWIKGHFDY (SEQ ID NO:801) respectively, and/or a light chain with three CDRs comprising the amino acid sequences GGSIASNY (SEQ ID NO:847), EDN (SEQ ID NO:848), and QSYDSSNPVV (SEQ ID NO:849) respectively. In embodiments, the monoclonal antibody comprises a heavy chain with three CDRs comprising the amino acid sequences GFSLTTSGVS (SEQ ID NO:983), IHWDDDK (SEQ ID NO:984), and ASFIMTVYAEYFED (SEQ ID NO:985) respectively, and/or a light chain with three CDRs comprising the amino acid sequences QSVSSN (SEQ ID NO:986), DVS (SEQ ID NO:987), and QQRGAWPLT (SEQ ID NO:988) respectively.

In other embodiments, the monoclonal antibody comprises:
a. a V_{H} amino acid sequence having SEQ ID NO: 1, and a V_{L} amino acid sequence having SEQ ID NO: 2;
b. a V_{H} amino acid sequence having SEQ ID NO: 3, and a V_{L} amino acid sequence having SEQ ID NO: 4;
c. a V_{H} amino acid sequence having SEQ ID NO: 5, and a V_{L} amino acid sequence having SEQ ID NO: 6;
d. a V_{H} amino acid sequence having SEQ ID NO: 7, and a V_{L} amino acid sequence having SEQ ID NO: 8;
e. a V_{H} amino acid sequence having SEQ ID NO: 9, and a V_{L} amino acid sequence having SEQ ID NO: 10;
f. a V_{H} amino acid sequence having SEQ ID NO: 11, and a V_{L} amino acid sequence having SEQ ID NO: 12;
g. a V_{H} amino acid sequence having SEQ ID NO: 13, and a V_{L} amino acid sequence having SEQ ID NO: 14;
h. a V_{H} amino acid sequence having SEQ ID NO: 19, and a V_{L} amino acid sequence having SEQ ID NO: 20;
i. a V_{H} amino acid sequence having SEQ ID NO: 21, and a V_{L} amino acid sequence having SEQ ID NO: 22;
j. a V_{H} amino acid sequence having SEQ ID NO: 23, and a V_{L} amino acid sequence having SEQ ID NO: 24;
k. a V_{H} amino acid sequence having SEQ ID NO: 25, and a V_{L} amino acid sequence having SEQ ID NO: 26;
l. a V_{H} amino acid sequence having SEQ ID NO: 29, and a V_{L} amino acid sequence having SEQ ID NO: 30;
m. a V_{H} amino acid sequence having SEQ ID NO: 31, and a V_{L} amino acid sequence having SEQ ID NO: 32;
n. a V_{H} amino acid sequence having SEQ ID NO: 33, and a V_{L} amino acid sequence having SEQ ID NO: 34;
o. a V_{H} amino acid sequence having SEQ ID NO: 35, and a V_{L} amino acid sequence having SEQ ID NO: 36;
p. a V_{H} amino acid sequence having SEQ ID NO: 37, and a V_{L} amino acid sequence having SEQ ID NO: 38;
q. a V_{H} amino acid sequence having SEQ ID NO: 39, and a V_{L} amino acid sequence having SEQ ID NO: 40;
r. a V_{H} amino acid sequence having SEQ ID NO: 41, and a V_{L} amino acid sequence having SEQ ID NO: 42;
s. a V_{H} amino acid sequence having SEQ ID NO: 43, and a V_{L} amino acid sequence having SEQ ID NO: 44; or
t. a V_{H} amino acid sequence having SEQ ID NO: 47, and a V_{L} amino acid sequence having SEQ ID NO: 48.

In some embodiments, the antibody comprises: (a) a V_{H} amino acid sequence having SEQ ID NO: 15, and a V_{L} amino acid sequence having SEQ ID NO: 16; (b) a V_{H} amino acid sequence having SEQ ID NO: 17, and a V_{L} amino acid sequence having SEQ ID NO: 18; or (c) a V_{H} amino acid sequence having SEQ ID NO: 27, and a V_{L} amino acid sequence having SEQ ID NO: 28. In other embodiments, the monoclonal antibody comprises:
a. a V_{H} amino acid sequence having SEQ ID NO: 49, and a V_{L} amino acid sequence having SEQ ID NO: 50;
b. a V_{H} amino acid sequence having SEQ ID NO: 51, and a V_{L} amino acid sequence having SEQ ID NO: 52;
c. a V_{H} amino acid sequence having SEQ ID NO: 53, and a V_{L} amino acid sequence having SEQ ID NO: 54;
d. a V_{H} amino acid sequence having SEQ ID NO: 55, and a V_{L} amino acid sequence having SEQ ID NO: 56;
e. a V_{H} amino acid sequence having SEQ ID NO: 57, and a V_{L} amino acid sequence having SEQ ID NO: 58;
f. a V_{H} amino acid sequence having SEQ ID NO: 59, and a V_{L} amino acid sequence having SEQ ID NO: 60;
g. a V_{H} amino acid sequence having SEQ ID NO: 61, and a V_{L} amino acid sequence having SEQ ID NO: 62;
h. a V_{H} amino acid sequence having SEQ ID NO: 63, and a V_{L} amino acid sequence having SEQ ID NO: 64;
i. a V_{H} amino acid sequence having SEQ ID NO: 65, and a V_{L} amino acid sequence having SEQ ID NO: 66;
j. a V_{H} amino acid sequence having SEQ ID NO: 67, and a V_{L} amino acid sequence having SEQ ID NO: 68;
k. a V_{H} amino acid sequence having SEQ ID NO: 69, and a V_{L} amino acid sequence having SEQ ID NO: 70;
l. a V_{H} amino acid sequence having SEQ ID NO: 71, and a V_{L} amino acid sequence having SEQ ID NO: 72;
m. a V_{H} amino acid sequence having SEQ ID NO: 73, and a V_{L} amino acid sequence having SEQ ID NO: 74;
n. a V_{H} amino acid sequence having SEQ ID NO: 75, and a V_{L} amino acid sequence having SEQ ID NO: 76;
o. a V_{H} amino acid sequence having SEQ ID NO: 77, and a V_{L} amino acid sequence having SEQ ID NO: 78;
p. a V_{H} amino acid sequence having SEQ ID NO: 79, and a V_{L} amino acid sequence having SEQ ID NO: 80;
q. a V_{H} amino acid sequence having SEQ ID NO: 81, and a V_{L} amino acid sequence having SEQ ID NO: 82;
r. a V_{H} amino acid sequence having SEQ ID NO: 83, and a V_{L} amino acid sequence having SEQ ID NO: 84;
s. a V_{H} amino acid sequence having SEQ ID NO: 85, and a V_{L} amino acid sequence having SEQ ID NO: 86;
t. a V_{H} amino acid sequence having SEQ ID NO: 87, and a V_{L} amino acid sequence having SEQ ID NO: 88;
u. a V_{H} amino acid sequence having SEQ ID NO: 89, and a V_{L} amino acid sequence having SEQ ID NO: 90; or
v. a V_{H} amino acid sequence having SEQ ID NO: 91, and a V_{L} amino acid sequence having SEQ ID NO: 92.

In some embodiments, the antibody comprises: a) a VH amino acid sequence having SEQ ID NO: 722, and a VL amino acid sequence having SEQ ID NO: 723; b) a VH amino acid sequence having SEQ ID NO: 724, and a VL amino acid sequence having SEQ ID NO: 725; c) a VH amino acid sequence having SEQ ID NO: 726, and a VL amino acid sequence having SEQ ID NO: 727; d) a VH amino acid sequence having SEQ ID NO: 728, and a VL amino acid sequence having SEQ ID NO: 729; e) a VH amino acid sequence having SEQ ID NO: 730, and a VL amino acid sequence having SEQ ID NO: 731; f) a VH amino acid sequence having SEQ ID NO: 732, and a VL amino acid sequence having SEQ ID NO: 733; g) a VH amino acid sequence having SEQ ID NO: 734, and a VL amino acid sequence having SEQ ID NO: 735; h) a VH amino acid sequence having SEQ ID NO: 736, and a VL amino acid sequence having SEQ ID NO: 737; i) a VH amino acid sequence having SEQ ID NO: 738, and a VL amino acid sequence having SEQ ID NO: 739; j) a VH amino acid sequence having SEQ ID NO: 740, and a VL amino acid sequence having SEQ ID NO: 741; k) a VH amino acid sequence having SEQ ID NO: 742, and a VL amino acid sequence having SEQ ID NO: 743; 1) a VH amino acid sequence having SEQ ID NO: 744, and a VL amino acid sequence having SEQ ID NO: 745; m) a VH amino acid sequence having SEQ ID NO: 746, and a VL amino acid sequence having SEQ ID NO: 747; n) a VH amino acid sequence having SEQ ID NO: 748, and a VL amino acid sequence having SEQ ID NO: 749; o) a VH amino acid sequence having SEQ ID NO: 750, and a VL amino acid sequence having SEQ ID NO: 751; or p) a VH amino acid sequence having SEQ ID NO: 752, and a VL amino acid sequence having SEQ ID NO: 753. In embodiments, the monoclonal antibody comprises a VH amino acid sequence having SEQ ID NO: 981, and a V_{L} amino acid sequence having SEQ ID NO: 982. In one embodiment, the antibody is a multispecific antibody (for example, a bispecific antibody or trispecific antibody).

An aspect of the invention is directed to isolated scFv antibodies directed to Severe Acute Respiratory Syndrome coronavirus (SARS-CoV2). In some embodiments, the antibody binds to an epitope in SEQ ID NO: 979. In some embodiments, the scFv antibody binds to an epitope in the receptor binding domain (RBD) of the spike protein of SARS-CoV2. In other embodiments, the scFv antibody neutralizes SARS-CoV2. In some embodiments the epitope is linear. In other embodiments, the epitope is non-linear. In some embodiments, the epitope comprises a region within amino acids 319-490 of SEQ ID NO: 980 of the spike protein. In other embodiments, the epitope comprises a region within amino acids 319-541 SEQ ID NO: 980 of the spike protein. In further embodiments, the scFv antibody inhibits viral and cell membrane fusion. In yet other embodiments, the scFv antibody competes with the binding of a monoclonal antibody to the spike protein. In some embodiments, the scFv antibody blocks the binding of SARS-CoV2 spike protein to angiotensin converting enzyme 2 (ACE2) cell surface receptor. In another embodiment, the scFv antibody is a fully human antibody. In some embodiments, the scFv antibody comprises: (a) a heavy chain with three CDRs comprising the amino acid sequences GGSIRTHS (SEQ ID NO:93), IHHSGAT (SEQ ID NO:94), and ARGPGILSY (SEQ ID NO:95) respectively, and/or a light chain with three CDRs comprising the amino acid sequences SSNIGSNT (SEQ ID NO:227), SNN (SEQ ID NO:228), and AAWDDSLNVHYV (SEQ ID NO:229) respectively; (b) a heavy chain with three CDRs comprising the amino acid sequences GGSISSYY (SEQ ID NO:96), IYTSGST (SEQ ID NO:97), and ARDVGFGWFDR (SEQ ID NO:98) respectively and/or a light chain with three CDRs comprising the amino acid sequences SGSIASNY (SEQ ID NO:230), EDN (SEQ ID NO:231), and QSFDSASLWV (SEQ ID NO:232) respectively; (c) a heavy chain with three CDRs comprising the amino acid sequences GGSIRTHS (SEQ ID NO:99), IHHSGAT (SEQ ID NO:100), and ARGPGILSY (SEQ ID NO:101) respectively and/or a light chain with three CDRs comprising the amino acid sequences SSNIGSND (SEQ ID NO:233), SNN (SEQ ID NO:234), and ATWDDSLSAGV (SEQ ID NO:235) respectively; (d) a heavy chain with three CDRs comprising the amino acid sequences GDSVSSYSDA (SEQ ID NO:102), TYYRSKWYN (SEQ ID NO:103), and AREIVATTPFRNYYYGMDV (SEQ ID NO:104) respectively and/or a light chain with three CDRs comprising the amino acid sequences SGSIASNY (SEQ ID NO:236), QDK (SEQ ID NO:237), and QSYDSSSLWV (SEQ ID NO:238) respectively; (e) a heavy chain with three CDRs comprising the amino acid sequences GFTFSHYD (SEQ ID NO:105), IGYDGTNL (SEQ ID NO: 106), and ARAANYYDSSGYGRADAFDI (SEQ ID NO:107) respectively and/or a light chain with three CDRs comprising the amino acid sequences TGSIAGNY (SEQ ID NO:239), DDN (SEQ ID NO:240), and QSYDSGNRGV (SEQ ID NO:241) respectively; (f) a heavy chain with three CDRs comprising the amino acid sequences GFTFSDFP (SEQ ID NO:108), ISYDGNIK (SEQ ID NO:109), and ARGGSSFDI (SEQ ID NO:110) respectively and/or a light chain with three CDRs comprising the amino acid sequences TSNIGNNA (SEQ ID NO:242), YNE (SEQ ID NO:243), and AAWDDSLSGHVV (SEQ ID NO:244) respectively; (g) a heavy chain with three CDRs comprising the amino acid sequences GFSLSTTGVG (SEQ ID NO:111), IYWNDDK (SEQ ID NO:112), and ARISGSGYFYPFDI (SEQ ID NO:113) respectively and/or a light chain with three CDRs comprising the amino acid sequences SGSIASNY (SEQ ID NO:245), EDN (SEQ ID NO:246), and QSYDSSSLWV (SEQ ID NO:247) respectively; (h) a heavy chain with three CDRs comprising the amino acid sequences GYTFSDYY (SEQ ID NO: 120), IDPNSGGT (SEQ ID NO:121), and ARDRGRGGQAGAFDY (SEQ ID NO:978) respectively and/or a light chain with three CDRs comprising the amino acid sequences KIGSKS (SEQ ID NO:254), DDS (SEQ ID NO:255), and HVWDSSSDQNV (SEQ ID NO:256) respectively; (i) a heavy chain with three CDRs comprising the amino acid sequences GFTFSSYA (SEQ ID NO:122), ISYGGSNK (SEQ ID NO:123), and AKVRGSGWYWGSAFDI (SEQ ID NO:124) respectively and/or a light chain with three CDRs comprising the amino acid sequences SLRAYF (SEQ ID NO:257), GQD (SEQ ID NO:258), and NSRDSGENHLI (SEQ ID NO:259) respectively; (j) a heavy chain with three CDRs comprising the amino acid sequences GYSFTGSH (SEQ ID NO:125), INPDSGVI (SEQ ID NO:126), and ARDKAIGYVWALDY (SEQ ID NO:127) respectively and/or a light chain with three CDRs comprising the amino acid sequences SSDVGTYNR (SEQ ID NO:260), EVS (SEQ ID NO:261), and SSYTRTFTYV (SEQ ID NO:262) respectively; (k) a heavy chain with three CDRs comprising the amino acid sequences GVSLDTIGMR (SEQ ID NO:128), IDWDDDK (SEQ ID NO: 129), and ARSGLLYDLDV (SEQ ID NO:130) respectively and/or a light chain with three CDRs comprising the amino acid sequences DSDIGANF (SEQ ID NO:263), RNT (SEQ ID NO:264), and QSYDSSLSAYV (SEQ ID NO:265) respectively; (l) a heavy chain with three CDRs comprising the amino acid sequences GYSFTSYW (SEQ ID NO:134), IYPGDSDT (SEQ ID NO:135), and ARGWQWHDY (SEQ ID NO:136) respectively and/or a light chain with three CDRs comprising the amino acid sequences SLRSYY (SEQ ID NO:269), DKD (SEQ ID NO:270), and NSRDRSDNHVV (SEQ ID NO:271) respectively; (m) a heavy chain with three CDRs comprising the amino acid sequences GDSVSSRSSA (SEQ ID NO:137), TYYRSNWNY (SEQ ID NO:138), and VRNMRPDFDL (SEQ ID NO:139) respectively and/or a light chain with three CDRs comprising the amino acid sequences QSVSNN (SEQ ID NO:272), DAT (SEQ ID NO:273), and QQYDNLPV (SEQ ID NO:274) respectively; (n) a heavy chain with three CDRs comprising the amino acid sequences GYTFTTSG (SEQ ID NO:140), ISAYNGNT (SEQ ID NO:141), and ARDFHLYYGMDV (SEQ ID NO:142) respectively and/or a light chain with three CDRs comprising the amino acid sequences SSDVGAYNY (SEQ ID NO:275), DVT (SEQ ID NO:276), and AVWDDGLNGRVV (SEQ ID NO:277) respectively; (o) a heavy chain with three CDRs comprising the amino acid sequences GGTFSSYA (SEQ ID NO:143), INPNSGGT (SEQ ID NO:144), and ARGSGGYYLG (SEQ ID NO:145) respectively and/or a light chain with three CDRs comprising the amino acid sequences SNNVGNQG (SEQ ID NO:278), MNN (SEQ ID NO:279), and SAWDSSLSRWV (SEQ ID NO:280) respectively; (p) a heavy chain with three CDRs comprising the amino acid sequences GGTFSSYT (SEQ ID NO: 146), IIPILGTP (SEQ ID NO: 147), and AVGSGWYSGFDY (SEQ ID NO:148) respectively and/or a light chain with three CDRs comprising the amino acid sequences SGSIASNY (SEQ ID NO:281), EDS (SEQ ID NO:282), and QSFHNSNPVI (SEQ ID NO:283) respectively; (q) a heavy chain with three CDRs comprising the amino acid sequences GFTFSSYW (SEQ ID NO:149), IKQDGSEK (SEQ ID NO:150), and ARGFYYYGAFDI (SEQ ID NO:151) respectively and/or a light chain with three CDRs comprising the amino acid sequences SGSIASNY (SEQ ID NO:284), EDN (SEQ ID NO:285), and QSYDSSNHWV (SEQ ID NO:286) respectively; (r) a heavy chain with three CDRs comprising the amino acid sequences GFTFDDYA (SEQ ID NO:152), IDWNSGVI (SEQ ID NO:153), and AKDAYSYGFLGAFDI (SEQ ID NO:154) respectively and/or a light chain with three CDRs comprising the amino acid sequences NIGSKS (SEQ ID NO:287), EDR (SEQ ID NO:288), and QVWDGDSDHYV (SEQ ID NO:289) respectively; (s) a heavy chain with three CDRs comprising the amino acid sequences GFTFDDYA (SEQ ID NO:155), IDWNSGVI (SEQ ID NO:156), and ARDILPSNFDGKKIIVFQPPAKRDLDNYYGMDV (SEQ ID NO:157) respectively and/or a light chain with three CDRs comprising the amino acid sequences SSDVGGYNL (SEQ ID NO:290), EGS (SEQ ID NO:291), and SSYTITDVVV (SEQ ID NO:292) respectively; or (t) a heavy chain with three CDRs comprising the amino acid sequences GYSFTSNW (SEQ ID NO:158), IFPGDSDT (SEQ ID NO:159), and ARESYNAYGS (SEQ ID NO:160) respectively and/or a light chain with three CDRs comprising the amino acid sequences SSNIGSNP (SEQ ID NO:293), SNN (SEQ ID NO:294), and AAWDDSLSGVV (SEQ ID NO:295) respectively.

In other embodiments, the scFv antibody comprises: (a) a heavy chain with three CDRs comprising the amino acid sequences GFTFTTYG (SEQ ID NO:114), ISYDGSIK (SEQ ID NO:115), and ARVGDSSSYYGIDA (SEQ ID NO:116) respectively, and/or a light chain with three CDRs comprising the amino acid sequences SSNIGSNS (SEQ ID NO:248), SNN (SEQ ID NO:249), and AAWDDSLTGYV (SEQ ID NO:250) respectively; (b) a heavy chain with three CDRs comprising the amino acid sequences GFTFSSHA (SEQ ID NO:117), ISYDGSYT (SEQ ID NO:118), and ARDWVNFGMDV (SEQ ID NO:119) respectively, and/or a light chain with three CDRs comprising the amino acid sequences SSDVGGYNY (SEQ ID NO:251), EVS (SEQ ID NO:252), and AAWDDSLSGPV (SEQ ID NO:253) respectively; or (c) a heavy chain with three CDRs comprising the amino acid sequences GFTFSDYP (SEQ ID NO:131), TSYDGRIK (SEQ ID NO:132), and ARDPGWLRSVGMDV (SEQ ID NO:133) respectively, and/or a light chain with three CDRs comprising the amino acid sequences SGSIARNY (SEQ ID NO:266), ADR (SEQ ID NO:267), and QSYDSSNQAAV (SEQ ID NO:268) respectively. In yet further embodiments, the scFv antibody comprises:
a) a heavy chain with three CDRs comprising the amino acid sequences GYTFTSYG (SEQ ID NO:161), ISAYNGNT (SEQ ID NO:162), and ARGFPQLGSDY (SEQ ID NO: 163) respectively, and/or a light chain with three CDRs comprising the amino acid sequences SGSIASNY (SEQ ID NO:296), EDN (SEQ ID NO:297), and QSYDSTNWV (SEQ ID NO:298) respectively;
b) a heavy chain with three CDRs comprising the amino acid sequences GGTFSSYA (SEQ ID NO:164), ISGYNGNT (SEQ ID NO:165), and ARQMKDSGNYWEYYYYGMDV (SEQ ID NO:166) respectively, and/or a light chain with three CDRs comprising the amino acid sequences NIGSES (SEQ ID NO:299), EDR (SEQ ID NO:300), and QVWNPSGSLQYV (SEQ ID NO:301) respectively;
c) a heavy chain with three CDRs comprising the amino acid sequences GYTFTSYG (SEQ ID NO:167), ISTYNGNT (SEQ ID NO:168), and ARDVFGHFDY (SEQ ID NO:169) respectively, and/or a light chain with three CDRs comprising the amino acid sequences SGNIATNY (SEQ ID NO:302), EDN (SEQ ID NO:303), and KSYDDGNHV (SEQ ID NO:304) respectively;
d) a heavy chain with three CDRs comprising the amino acid sequences GFSLTTTGVS (SEQ ID NO:170), IHWDDDK (SEQ ID NO:171), and ASFIMTVYAEYFED (SEQ ID NO: 172) respectively, and/or a light chain with three CDRs comprising the amino acid sequences QSVSSN (SEQ ID NO:305), DVS (SEQ ID NO:306), and QQRGVWPLT (SEQ ID NO:307) respectively;
e) a heavy chain with three CDRs comprising the amino acid sequences GFSLSTSAMC (SEQ ID NO:173), IDWDNDR (SEQ ID NO:174), and AHSPYDSIWGSFRPSVYYFDY (SEQ ID NO:175) respectively, and/or a light chain with three CDRs comprising the amino acid sequences SGSIVSSY (SEQ ID NO:308), EHN (SEQ ID NO:309), and QSYDSQNGV (SEQ ID NO:310) respectively;
f) a heavy chain with three CDRs comprising the amino acid sequences GFTFSDYY (SEQ ID NO:176), ISSSSSDT (SEQ ID NO:177), and AMPTREPAY (SEQ ID NO:178) respectively, and/or a light chain with three CDRs comprising the amino acid sequences SSDLGTYNY (SEQ ID NO:311), DVF (SEQ ID NO:312), and SSYTSSSTYV (SEQ ID NO:313) respectively;
g) a heavy chain with three CDRs comprising the amino acid sequences GFAFSDFP (SEQ ID NO: 179), ISYDGSLK (SEQ ID NO:180), and AREGVSNSRPFDH (SEQ ID NO:181) respectively, and/or a light chain with three CDRs comprising the amino acid sequences SIGTKS (SEQ ID NO:314), DDD (SEQ ID NO:315), and QVWESDDDDLV (SEQ ID NO:316) respectively;
h) a heavy chain with three CDRs comprising the amino acid sequences GFTFSSYA (SEQ ID NO:182), ISSNGGST (SEQ ID NO:183), and TRDLWSGSADSFDI (SEQ ID NO:184) respectively, and/or a light chain with three CDRs comprising the amino acid sequences SLRRYY (SEQ ID NO: 317), GKN (SEQ ID NO:318), and NSRDISDNQWQWI (SEQ ID NO:319) respectively;
i) a heavy chain with three CDRs comprising the amino acid sequences GFPFNAYY (SEQ ID NO:185), INQDGSEK (SEQ ID NO:186), and ARLYWWGMDV (SEQ ID NO:187) respectively, and/or a light chain with three CDRs comprising the amino acid sequences SSDVGGYKY (SEQ ID NO:320), DVN (SEQ ID NO:321), and SSYTGRMNLYV (SEQ ID NO:322) respectively;
j) a heavy chain with three CDRs comprising the amino acid sequences GFTFDDYA (SEQ ID NO:188), IDWNSGVI (SEQ ID NO:189), and AKDAYSYGFLGAFDI (SEQ ID NO:190) respectively, and/or a light chain with three CDRs comprising the amino acid sequences NIRTKG (SEQ ID NO:323), YAS (SEQ ID NO:324), and QVWDSSSDLVV (SEQ ID NO:325) respectively;
k) a heavy chain with three CDRs comprising the amino acid sequences GFTFDDYA (SEQ ID NO:191), ISWNSGSI (SEQ ID NO:192), and ARDWWGSIDH (SEQ ID NO:193) respectively, and/or a light chain with three CDRs comprising the amino acid sequences SSDVGGYDY (SEQ ID NO:326), DVS (SEQ ID NO:327), and SSYTSSSPVV (SEQ ID NO:328) respectively;
l) a heavy chain with three CDRs comprising the amino acid sequences GGSISSSNW (SEQ ID NO:194), IYHSGST (SEQ ID NO:195), and ARRGGTYHRGAFDI (SEQ ID NO:196) respectively, and/or a light chain with three CDRs comprising the amino acid sequences SRDVGSYDL (SEQ ID NO:329), EGS (SEQ ID NO:330), and SSYTSSNSLV (SEQ ID NO:331) respectively;
m) a heavy chain with three CDRs comprising the amino acid sequences GASISNSF (SEQ ID NO:197), TSYSGNS (SEQ ID NO:198), and ARREWIKGHFDY (SEQ ID NO:199) respectively, and/or a light chain with three CDRs comprising the amino acid sequences GGSIASNY (SEQ ID NO:332), EDN (SEQ ID NO:333), and QSYDSSNPVV (SEQ ID NO:334) respectively;
n) a heavy chain with three CDRs comprising the amino acid sequences GGSFTTHS (SEQ ID NO:200), ILPGGAT (SEQ ID NO:201), and ARGPGILSY (SEQ ID NO:202) respectively, and/or a light chain with three CDRs comprising the amino acid sequences SSIGSND (SEQ ID NO:335), SNN (SEQ ID NO:336), and AWDDSLSAVV (SEQ ID NO:337) respectively;
o) a heavy chain with three CDRs comprising the amino acid sequences GGSFRTHS (SEQ ID NO:203), IHHSGAT (SEQ ID NO:204), and ARGPGILSY (SEQ ID NO:205) respectively, and/or a light chain with three CDRs comprising the amino acid sequences SSNIGSNT (SEQ ID NO:338), INN (SEQ ID NO:339), and AEWYDSLNVHYV (SEQ ID NO:340) respectively;
p) a heavy chain with three CDRs comprising the amino acid sequences GGSIRTHS (SEQ ID NO:206), IHHSGAT (SEQ ID NO:207), and ARGPGILSY (SEQ ID NO:208) respectively, and/or a light chain with three CDRs comprising the amino acid sequences SSNIGSNT (SEQ ID NO:341), INN (SEQ ID NO:342), and AECYDSLNDHYV (SEQ ID NO:343) respectively;
q) a heavy chain with three CDRs comprising the amino acid sequences GGSIRTHS (SEQ ID NO:209), IHHSGAT (SEQ ID NO:210), and GRGPGILSY (SEQ ID NO:211) respectively, and/or a light chain with three CDRs comprising the amino acid sequences SSNIGSNT (SEQ ID NO:344), SNN (SEQ ID NO:345), and AAWDDSLNVHYV (SEQ ID NO:346) respectively;
r) a heavy chain with three CDRs comprising the amino acid sequences GYSFTSYW (SEQ ID NO:212), IYPGDSDT (SEQ ID NO:213), and ARQGDGGGYDY (SEQ ID NO:214) respectively, and/or a light chain with three CDRs comprising the amino acid sequences SSNIGSNP (SEQ ID NO:347), NNN (SEQ ID NO:348), and AAWDDSLNGL (SEQ ID NO:349) respectively;
s) a heavy chain with three CDRs comprising the amino acid sequences RYSFSNYW (SEQ ID NO:215), IYPYDSDT (SEQ ID NO:216), and ARQGSSQSFDI (SEQ ID NO:217) respectively, and/or a light chain with three CDRs comprising the amino acid sequences SLRSYY (SEQ ID NO:350), GKN (SEQ ID NO:351), and NSRDSSGDVRV (SEQ ID NO:352) respectively;
t) a heavy chain with three CDRs comprising the amino acid sequences GYSFTSYW (SEQ ID NO:218), IYPGDSDT (SEQ ID NO:219), and ARRRGSAAAFDT (SEQ ID NO:220) respectively, and/or a light chain with three CDRs comprising the amino acid sequences SSNIGSNP (SEQ ID NO:353), DNN (SEQ ID NO:354), and EAWDDSLSGPV (SEQ ID NO:355) respectively;
u) a heavy chain with three CDRs comprising the amino acid sequences GYSFTSYW (SEQ ID NO:221), IYPGDSDT (SEQ ID NO:222), and ARTTYSYGSFDY (SEQ ID NO:223) respectively, and/or a light chain with three CDRs comprising the amino acid sequences SSNIGGNS (SEQ ID NO:356), RNN (SEQ ID NO:357), and AAWDDSLNGWV (SEQ ID NO:358) respectively; or
v) a heavy chain with three CDRs comprising the amino acid sequences GDSVTSNSAA (SEQ ID NO:224), TYYSSKWYN (SEQ ID NO:225), and ARGWLRLSFDP (SEQ ID NO:226) respectively, and/or a light chain with three CDRs comprising the amino acid sequences SGSIASNY (SEQ ID NO:359), EDN (SEQ ID NO:360), and QSYDPNNHGVV (SEQ ID NO:361) respectively.

In other embodiments, the scFv antibody comprises: a) a heavy chain with three CDRs comprising the amino acid sequences GFSLSTSGVG (SEQ ID NO:754), IYWDDDK (SEQ ID NO:755), and ARISGSGYFYPFDI (SEQ ID NO:756) respectively, and/or a light chain with three CDRs comprising the amino acid sequences SGSIASNY (SEQ ID NO:802), EDN (SEQ ID NO:803), and QSYDSSNLWV (SEQ ID NO:804) respectively; b) a heavy chain with three CDRs comprising the amino acid sequences GDSVSSNSAA (SEQ ID NO:757), TYYRSRWYN (SEQ ID NO:758), and AREIRGFDY (SEQ ID NO:759) respectively, and/or a light chain with three CDRs comprising the amino acid sequences SSDVGAYNF (SEQ ID NO:805), DFN (SEQ ID NO:806), and SSYAGSNNFDVV (SEQ ID NO:807) respectively; c) a heavy chain with three CDRs comprising the amino acid sequences GFTFGDYA (SEQ ID NO:760), IRSKAYGGTT (SEQ ID NO:761), and TTADDDMDV (SEQ ID NO:762) respectively, and/or a light chain with three CDRs comprising the amino acid sequences SGTIASNY (SEQ ID NO:808), EDN (SEQ ID NO:809), and QSYDTSNHYV (SEQ ID NO:810) respectively; d) a heavy chain with three CDRs comprising the amino acid sequences GFTFSNYG (SEQ ID NO:763), IWERGSKK (SEQ ID NO:764), and AREGISMTGAEYFQH (SEQ ID NO:765) respectively, and/or a light chain with three CDRs comprising the amino acid sequences SSNIGAGYD (SEQ ID NO:811), GTN (SEQ ID NO:812), and QSYDNSLTDPYV (SEQ ID NO:813) respectively; e) a heavy chain with three CDRs comprising the amino acid sequences GFTFDDYA (SEQ ID NO:766), IDWNSGVI (SEQ ID NO:767), and AKDIGPGGSGSYYAFDI (SEQ ID NO:768) respectively, and/or a light chain with three CDRs comprising the amino acid sequences SSDVGGSKY (SEQ ID NO:814), DVT (SEQ ID NO:815), and AAWDDSLNGVV (SEQ ID NO:816) respectively; f) a heavy chain with three CDRs comprising the amino acid sequences GFSFSRYG (SEQ ID NO:769), IRHDGSKK (SEQ ID NO:770), and AKDGRLEAALDD (SEQ ID NO:771) respectively, and/or a light chain with three CDRs comprising the amino acid sequences SGSIANNF (SEQ ID NO:817), EDN (SEQ ID NO:818), and QSYDSSNLV (SEQ ID NO:819) respectively; g) a heavy chain with three CDRs comprising the amino acid sequences GYSFTSYW (SEQ ID NO:772), IYPGDSDT (SEQ ID NO:773), and ARRGDLDAFDI (SEQ ID NO:774) respectively, and/or a light chain with three CDRs comprising the amino acid sequences SANIGSNA (SEQ ID NO:820), GNT (SEQ ID NO:821), and AAWDDSLNGYV (SEQ ID NO:822) respectively; h) a heavy chain with three CDRs comprising the amino acid sequences GYRLSDYY (SEQ ID NO:775), IKQDGSEK (SEQ ID NO:776), and ARVRGWSRGYFDY (SEQ ID NO:777) respectively, and/or a light chain with three CDRs comprising the amino acid sequences SGSIASNY (SEQ ID NO:823), EDN (SEQ ID NO:824), and QSYDSSNHWV (SEQ ID NO:825) respectively; i) a heavy chain with three CDRs comprising the amino acid sequences GFTFDDYA (SEQ ID NO:778), ISWNSGSI (SEQ ID NO:779), and ARDWWGSIDH (SEQ ID NO:780) respectively, and/or a light chain with three CDRs comprising the amino acid sequences SSDVGGYDY (SEQ ID NO:826), DVS (SEQ ID NO:827), and SSYTSSSPVV (SEQ ID NO:828) respectively; j) a heavy chain with three CDRs comprising the amino acid sequences GFTFSHYD (SEQ ID NO:781), IGYDGTNL (SEQ ID NO:782), and ARAANYYDSSGYGRADAFDI (SEQ ID NO:783) respectively, and/or a light chain with three CDRs comprising the amino acid sequences TGSIAGNY (SEQ ID NO:829), DDN (SEQ ID NO:830), and QSYDSGNRGV (SEQ ID NO:831) respectively; k) a heavy chain with three CDRs comprising the amino acid sequences GGTFSTYG (SEQ ID NO:784), IIPSLGIP (SEQ ID NO:785), and ARENIDLATNDF (SEQ ID NO:786) respectively, and/or a light chain with three CDRs comprising the amino acid sequences SRDIGAYGY (SEQ ID NO:832), EVR (SEQ ID NO:833), and SSYTSSSTLDVV (SEQ ID NO:834) respectively; l) a heavy chain with three CDRs comprising the amino acid sequences GGTFSSSG (SEQ ID NO:787), IIPMLGTP (SEQ ID NO:788), and ARDGGNYDY (SEQ ID NO:789) respectively, and/or a light chain with three CDRs comprising the amino acid sequences SSNIGRNA (SEQ ID NO:835), SNN (SEQ ID NO:836), and SAWDTSLSTWV (SEQ ID NO:837) respectively; m) a heavy chain with three CDRs comprising the amino acid sequences GFTFSSYW (SEQ ID NO:790), IKQDGSEK (SEQ ID NO:791), and ARGFYYYGAFDI (SEQ ID NO:792) respectively, and/or a light chain with three CDRs comprising the amino acid sequences SGSIASNY (SEQ ID NO:838), EDN (SEQ ID NO:839), and QSYDSSNHWV (SEQ ID NO:840) respectively; n) a heavy chain with three CDRs comprising the amino acid sequences GFTFDDYA (SEQ ID NO:793), IDWNSGVI (SEQ ID NO:794), and AKDAYSYGFLGAFDI (SEQ ID NO:795) respectively, and/or a light chain with three CDRs comprising the amino acid sequences NIRTKG (SEQ ID NO:841), YAS (SEQ ID NO:842), and QVWDSSSDLVV (SEQ ID NO:843) respectively; o) a heavy chain with three CDRs comprising the amino acid sequences GYSFTGSH (SEQ ID NO:796), INPDSGVI (SEQ ID NO:797), and ARDKAIGYVWALDY (SEQ ID NO:798) respectively, and/or a light chain with three CDRs comprising the amino acid sequences SSDVGTYNR (SEQ ID NO:844), EVS (SEQ ID NO:845), and SSYTRTFTYV (SEQ ID NO:846) respectively; or p) a heavy chain with three CDRs comprising the amino acid sequences GASISNSF (SEQ ID NO:799), TSYSGNS (SEQ ID NO:800), and ARREWIKGHFDY (SEQ ID NO:801) respectively, and/or a light chain with three CDRs comprising the amino acid sequences GGSIASNY (SEQ ID NO:847), EDN (SEQ ID NO:848), and QSYDSSNPVV (SEQ ID NO:849) respectively. In embodiments, the scFv antibody comprises a heavy chain with three CDRs comprising the amino acid sequences GFSLTTSGVS (SEQ ID NO:983), IHWDDDK (SEQ ID NO:984), and ASFIMTVYAEYFED (SEQ ID NO:985) respectively, and/or a light chain with three CDRs comprising the amino acid sequences QSVSSN (SEQ ID NO:986), DVS (SEQ ID NO:987), and QQRGAWPLT (SEQ ID NO:988) respectively

In other embodiments, the scFv antibody comprises:
a. a V_{H} amino acid sequence having SEQ ID NO: 1, and a V_{L} amino acid sequence having SEQ ID NO: 2;
b. a V_{H} amino acid sequence having SEQ ID NO: 3, and a V_{L} amino acid sequence having SEQ ID NO: 4;
c. a V_{H} amino acid sequence having SEQ ID NO: 5, and a V_{L} amino acid sequence having SEQ ID NO: 6;
d. a V_{H} amino acid sequence having SEQ ID NO: 7, and a V_{L} amino acid sequence having SEQ ID NO: 8;
e. a V_{H} amino acid sequence having SEQ ID NO: 9, and a V_{L} amino acid sequence having SEQ ID NO: 10;
f. a V_{H} amino acid sequence having SEQ ID NO: 11, and a V_{L} amino acid sequence having SEQ ID NO: 12;
g. a V_{H} amino acid sequence having SEQ ID NO: 13, and a V_{L} amino acid sequence having SEQ ID NO: 14;
h. a V_{H} amino acid sequence having SEQ ID NO: 19, and a V_{L} amino acid sequence having SEQ ID NO: 20;
i. a V_{H} amino acid sequence having SEQ ID NO: 21, and a V_{L} amino acid sequence having SEQ ID NO: 22;
j. a V_{H} amino acid sequence having SEQ ID NO: 23, and a V_{L} amino acid sequence having SEQ ID NO: 24;
k. a V_{H} amino acid sequence having SEQ ID NO: 25, and a V_{L} amino acid sequence having SEQ ID NO: 26;
l. a V_{H} amino acid sequence having SEQ ID NO: 29, and a V_{L} amino acid sequence having SEQ ID NO: 30;
m. a V_{H} amino acid sequence having SEQ ID NO: 31, and a V_{L} amino acid sequence having SEQ ID NO: 32;
n. a V_{H} amino acid sequence having SEQ ID NO: 33, and a V_{L} amino acid sequence having SEQ ID NO: 34;
o. a V_{H} amino acid sequence having SEQ ID NO: 35, and a V_{L} amino acid sequence having SEQ ID NO: 36;
p. a V_{H} amino acid sequence having SEQ ID NO: 37, and a V_{L} amino acid sequence having SEQ ID NO: 38;
q. a V_{H} amino acid sequence having SEQ ID NO: 39, and a V_{L} amino acid sequence having SEQ ID NO: 40;
r. a V_{H} amino acid sequence having SEQ ID NO: 41, and a V_{L} amino acid sequence having SEQ ID NO: 42;
s. a V_{H} amino acid sequence having SEQ ID NO: 43, and a V_{L} amino acid sequence having SEQ ID NO: 44; or
t. a V_{H} amino acid sequence having SEQ ID NO: 47, and a V_{L} amino acid sequence having SEQ ID NO: 48.

In some embodiments, the scFv antibody comprises: (a) a V_{H} amino acid sequence having SEQ ID NO: 15, and a V_{L} amino acid sequence having SEQ ID NO: 16; (b) a V_{H} amino acid sequence having SEQ ID NO: 17, and a V_{L} amino acid sequence having SEQ ID NO: 18; or (c) a V_{H} amino acid sequence having SEQ ID NO: 27, and a V_{L} amino acid sequence having SEQ ID NO: 28. In other embodiments, the scFv antibody comprises:
a. a V_{H} amino acid sequence having SEQ ID NO: 49, and a V_{L} amino acid sequence having SEQ ID NO: 50;
b. a V_{H} amino acid sequence having SEQ ID NO: 51, and a V_{L} amino acid sequence having SEQ ID NO: 52;
c. a V_{H} amino acid sequence having SEQ ID NO: 53, and a V_{L} amino acid sequence having SEQ ID NO: 54;
d. a V_{H} amino acid sequence having SEQ ID NO: 55, and a V_{L} amino acid sequence having SEQ ID NO: 56;
e. a V_{H} amino acid sequence having SEQ ID NO: 57, and a V_{L} amino acid sequence having SEQ ID NO: 58;
f. a V_{H} amino acid sequence having SEQ ID NO: 59, and a V_{L} amino acid sequence having SEQ ID NO: 60;
g. a V_{H} amino acid sequence having SEQ ID NO: 61, and a V_{L} amino acid sequence having SEQ ID NO: 62;
h. a V_{H} amino acid sequence having SEQ ID NO: 63, and a V_{L} amino acid sequence having SEQ ID NO: 64;
i. a V_{H} amino acid sequence having SEQ ID NO: 65, and a V_{L} amino acid sequence having SEQ ID NO: 66;
j. a V_{H} amino acid sequence having SEQ ID NO: 67, and a V_{L} amino acid sequence having SEQ ID NO: 68;
k. a V_{H} amino acid sequence having SEQ ID NO: 69, and a V_{L} amino acid sequence having SEQ ID NO: 70;
l. a V_{H} amino acid sequence having SEQ ID NO: 71, and a V_{L} amino acid sequence having SEQ ID NO: 72;
m. a V_{H} amino acid sequence having SEQ ID NO: 73, and a V_{L} amino acid sequence having SEQ ID NO: 74;
n. a V_{H} amino acid sequence having SEQ ID NO: 75, and a V_{L} amino acid sequence having SEQ ID NO: 76;
o. a V_{H} amino acid sequence having SEQ ID NO: 77, and a V_{L} amino acid sequence having SEQ ID NO: 78;
p. a V_{H} amino acid sequence having SEQ ID NO: 79, and a V_{L} amino acid sequence having SEQ ID NO: 80;
q. a V_{H} amino acid sequence having SEQ ID NO: 81, and a V_{L} amino acid sequence having SEQ ID NO: 82;
r. a V_{H} amino acid sequence having SEQ ID NO: 83, and a V_{L} amino acid sequence having SEQ ID NO: 84;
s. a V_{H} amino acid sequence having SEQ ID NO: 85, and a V_{L} amino acid sequence having SEQ ID NO: 86;
t. a V_{H} amino acid sequence having SEQ ID NO: 87, and a V_{L} amino acid sequence having SEQ ID NO: 88;
u. a V_{H} amino acid sequence having SEQ ID NO: 89, and a V_{L} amino acid sequence having SEQ ID NO: 90; or
v. a V_{H} amino acid sequence having SEQ ID NO: 91, and a V_{L} amino acid sequence having SEQ ID NO: 92.

In other embodiments, the scFv antibody comprises: a) a VH amino acid sequence having SEQ ID NO: 722, and a VL amino acid sequence having SEQ ID NO: 723; b) a VH amino acid sequence having SEQ ID NO: 724, and a VL amino acid sequence having SEQ ID NO: 725; c) a VH amino acid sequence having SEQ ID NO: 726, and a VL amino acid sequence having SEQ ID NO: 727; d) a VH amino acid sequence having SEQ ID NO: 728, and a VL amino acid sequence having SEQ ID NO: 729; e) a VH amino acid sequence having SEQ ID NO: 730, and a VL amino acid sequence having SEQ ID NO: 731; f) a VH amino acid sequence having SEQ ID NO: 732, and a VL amino acid sequence having SEQ ID NO: 733; g) a VH amino acid sequence having SEQ ID NO: 734, and a VL amino acid sequence having SEQ ID NO: 735; h) a VH amino acid sequence having SEQ ID NO: 736, and a VL amino acid sequence having SEQ ID NO: 737; i) a VH amino acid sequence having SEQ ID NO: 738, and a VL amino acid sequence having SEQ ID NO: 739;
j) a VH amino acid sequence having SEQ ID NO: 740, and a VL amino acid sequence having SEQ ID NO: 741; k) a VH amino acid sequence having SEQ ID NO: 742, and a VL amino acid sequence having SEQ ID NO: 743; 1) a VH amino acid sequence having SEQ ID NO: 744, and a VL amino acid sequence having SEQ ID NO: 745; m) a VH amino acid sequence having SEQ ID NO: 746, and a VL amino acid sequence having SEQ ID NO: 747;
n) a VH amino acid sequence having SEQ ID NO: 748, and a VL amino acid sequence having SEQ ID NO: 749; o) a VH amino acid sequence having SEQ ID NO: 750, and a VL amino acid sequence having SEQ ID NO: 751; or p) a VH amino acid sequence having SEQ ID NO: 752, and a VL amino acid sequence having SEQ ID NO: 753. In embodiments, the scFv antibody comprises a VH amino acid sequence having SEQ ID NO: 981, and a VL amino acid sequence having SEQ ID NO: 982. In one embodiment, the antibody is a multispecific antibody (for example, a bispecific antibody or trispecific antibody).

An aspect of the invention is directed to methods of preventing a disease or disorder caused by a Severe Acute Respiratory Syndrome coronavirus (SARS-CoV2). In some embodiments, the method comprises administering to a subject at risk of suffering from the disease or disorder, a therapeutically effective amount of the monoclonal antibody described herein or the scFv antibody described herein. In some embodiments, the method further comprises administering an anti-viral drug, a viral entry inhibitor, a viral attachment inhibitor, or a combination thereof. In some embodiments, the method comprises administering two or more antibodies specific to SARS-CoV2. In some embodiments, the antibody is administered prior to or after exposure to SARS-CoV2. In other embodiments, the antibody is administered at a dose sufficient to neutralize the SARS-CoV2. In one embodiment, the antibody is a multispecific antibody (for example, a bispecific antibody or trispecific antibody).

An aspect of the invention is directed to methods of delaying the onset of one or more symptoms of a SARS-CoV2 infection. In some embodiments, the method comprises administering to a subject at risk of suffering from the disease or disorder, a therapeutically effective amount of the monoclonal antibody described herein or the scFv antibody described herein. In some embodiments, the method further comprises administering an anti-viral drug, a viral entry inhibitor, a viral attachment inhibitor, or a combination thereof. In some embodiments, the method comprises administering two or more antibodies specific to SARS-CoV2. In some embodiments, the antibody is administered prior to or after exposure to SARS-CoV2. In other embodiments, the antibody is administered at a dose sufficient to neutralize the SARS-CoV2. In one embodiment, the antibody is a multispecific antibody (for example, a bispecific antibody or trispecific antibody).

An aspect of the invention is directed to compositions comprising the monoclonal antibody described herein or the scFv antibody described herein, and a carrier. In one embodiment, the antibody is a multispecific antibody (for example, a bispecific antibody or trispecific antibody).

An aspect of the invention is directed to methods of detecting the presence of SARS-CoV2 in a sample. In some embodiments, the method comprising: (a) contacting the sample with the monoclonal antibody described herein or the scFv antibody described herein; and detecting the presence or absence of an antibody-antigen complex, thereby detecting the presence of SARS-CoV2 in a sample. In some embodiments, the detecting occurs in vivo. In other embodiments, the sample is obtained from blood, hair, cheek scraping, saliva, biopsy, or semen.

Unless otherwise defined, all technical and scientific terms used herein can have the same meaning as understood by one of ordinary skill in the art to which this invention belongs. Although methods and materials similar or equivalent to those described herein can be used in the practice or testing of the invention, suitable methods and materials are described herein. All publications, patent applications, patents, and other references mentioned herein are incorporated by reference in their entirety. In the case of conflict, the specification, including definitions, will control. In addition, the materials, methods, and examples are illustrative only and are not intended to be limiting.

Other features and advantages of the invention will be apparent from and are encompassed by the following detailed description and claims.

### BRIEF DESCRIPTION OF THE DRAWINGS

FIG. 1A-FIG. 1P shows the amino acid sequences and germline assignments of the heavy chain and light chain regions of the antibodies directed to SARS-COV-2.

FIG. 2 shows the input and output phage titers from the 3 rounds of anti-SARS-COV-2 panning against soluble RBD or S1. An additional cross panning in round 3 with 2^{nd} round S1 phage applied to RBD was performed to further target the resultant phage to the RBD. As the phage clones enriched are dependent on the orientation and presentation of the bait (target protein), we expected that phage panned against S1 followed by RBD would yield additional clones compared to those selected after 3 rounds against RBD alone.

FIG. 3 shows screening results from the 3^{rd} round of panning.

FIG. 4 shows purified phage binding curves (RBD-Fc). The curves are made by coating plates with 1 μg/ml of SARS-COV-2 RBD-Fc or IL2-Fc (negative control) or blocking buffer only. Phage binding was detected by anti-M13-HRP; Negative curves plotted with the positive samples are against blocking buffer; Negative curves on the slides after are against IL2-Fc (1 μg/ml).

FIG. 5 shows EC50 values for purified phage against RBD-Fc. Red names had ambiguous curve fitting. Consult graphs for data reliability.

FIG. 6 shows Fc coat negative binding curves.

FIG. 7 shows purified phage binding against S1 protein. Negatives are also graphed.

FIG. 8 shows SARS-RBD-Fc ACE2 binding curve. These curves are made by coating plates with 1 μg/ml of SARS-COV-2 RBD-Fc and adding serial dilutions of ACE2-his tag. ACE2 binding is detected by HRP conjugated anti-his antibody.

FIG. 9 shows anti-RBD competition with ACE2. The red box on plate 1 shows exemplary clones of interest. These clones appear to demonstrate at least a partial ability to block RBD-ACE2 binding.

FIG. 10 shows a detailed look at the 7 anti-RBD clones that shows differential ELISA signal in blocking experiment. In this experiment, if the red bar is below that of the purple bar, it indicates that there is competition of the phage with ACE2.

FIG. 11 shows a RBD phage competition curve.

FIG. 12 shows the amino acid sequences of the heavy chain and light chain regions of the antibodies directed to SARS-COV-2. The asterisks are amber/stop codons. In the TG1 bacterial cells, they are mutated such that the TAG stop codon is read as a Q (glutamine). When the IMGT numbering is used to break the DNA sequence down into FW/CDRs, the system does not recognize an amber suppressor so a stop codon is assumed, but in the phage the codon is read as a Q. The sequences are later re-cloned such that the TAG is changed to the codons for Q. The periods are also from the IMGT system. In the numbering system they use, each FW/CDR needs to have a certain number of residues, so the periods are just used as gaps to make the beginning and the end of the segments fit their numbering scheme.

FIG. 13 is a table of KD measurements. KD values were measured on Octet with SA sensors. Abs are scFv-Fc format except for CR3022. Sensors were coated with 2.5 ug/ml Biotinylated SARS-CoV-2 S1 protein (ACRO, S1N-C82E8). Abs were run at 3 concentrations, 25-12.5-6.25 nM and the kinetic parameters were calculated by linking the three curves. Control sensor was coated with biotinylated PD1 and 25 nM antibody was allowed to bind.

FIG. 14 shows graphs of kinetic measurements.

FIG. 15 shows the nucleic acid sequences of the heavy chain and light chain regions of antibodies directed to SARS-COV-2.

FIG. 16 shows a phylogenetic tree of the coronavirus family and schematics of the viruses taken from Li, F. (2016). Structure, Function, and Evolution of Coronavirus Spike Proteins. Annu Rev Virol. http://doi.org/doi:10.1146/annurev-virology-110615-042301. The figure is an introduction to coronaviruses and their spike proteins. (a) Classification of coronaviruses. Representative coronaviruses in each genus are human coronavirus NL63 (HCoV-NL63), porcine transmissible gastroenteritis coronavirus (TGEV), porcine epidemic diarrhea coronavirus (PEDV), and porcine respiratory coronavirus (PRCV) in the genus Alphacoronavirus; severe acute respiratory syndrome coronavirus (SARS-CoV), Middle East respiratory syndrome coronavirus (MERS-CoV), bat coronavirus HKU4, mouse hepatitis coronavirus (MHV), bovine coronavirus (BCoV), and human coronavirus OC43 in the genus Betacoronavirus; avian infectious bronchitis coronavirus (IBV) in the genus Gammacoronavirus; and porcine deltacoronavirus (PdCV) in the genus Deltacoronavirus. (b) Schematic of the overall structure of prefusion coronavirus spikes. Shown are the receptor-binding subunit S1, the membrane-fusion subunit S2, the transmembrane anchor (TM), the intracellular tail (IC), and the viral envelope. (c) Schematic of the domain structure of coronavirus spikes, including the S1 N-terminal domain (S1-NTD), the S1 C-terminal domain (S1-CTD), the fusion peptide (FP), and heptad repeat regions N and C (HR-N and HR-C). Scissors indicate two proteolysis sites in coronavirus spikes. (d) Summary of the structures and functions of coronavirus spikes. Host receptors recognized by the S1 domains are angiotensin-converting enzyme 2 (ACE2), aminopeptidase N (APN), dipeptidyl peptidase 4 (DPP4), carcinoembryonic antigen-related cell adhesion molecule 1 (CEACAM1), and sugar. The available crystal structures of S1 domains and S2 HRs are shown. Their PDB IDs are 3KBH for HCoV-NL63 S1-CTD, 4F5C for PRCV S1-CTD, 2AJF for SARS-CoV S1-CTD, 4KR0 for MERS-CoV S1-CTD, 3R4D for MHV S1-NTD, 4H14 for BCoV S1-NTD, 2IEQ for HCoV-NL63 HRs, 1WYY for SARS-CoV HRs, 4NJL for MERS-CoV HRs, and 1WDF for MHV HRs.

FIG. 17 is a schematic of the coronavirus structure adapted from Li, F. (2016). Structure, Function, and Evolution of Coronavirus Spike Proteins. Annu Rev Virol. http://doi.org/doi:10.1146/annurev-virology-110615-042301.

FIG. 18 is a schematic of the structure of the 2019-nCoV S in the prefusion conformation adapted from Daniel Wrapp et al. Science 2020; 367:1260-1263. (A) Schematic of 2019-nCoV S primary structure colored by domain. Domains that were excluded from the ectodomain expression construct or cannot be visualized in the final map are colored white. SS, signal sequence; S2′, S2′ protease cleavage site; FP, fusion peptide; HR1, heptad repeat 1; CH, central helix; CD, connector domain; HR2, heptad repeat 2; TM, transmembrane domain; CT, cytoplasmic tail. Arrows denote protease cleavage sites. (B) Side and top views of the prefusion structure of the 2019-nCoV S protein with a single RBD in the up conformation. The two RBD down protomers are shown as cryo-EM density in white or gray and the RBD up protomer is shown in ribbons colored corresponding to the schematic in (A).

FIG. 19 is a schematic of ribbon diagrams showing the structural comparison between 2019-nCoV S and SARS-CoV Sadapted from Daniel Wrapp et al. Science 2020; 367:1260-1263. (A) Single protomer of 2019-nCoV S with the RBD in the down conformation (left) is shown in ribbons colored according to FIG. 1 of Wrapp et al. Science 2020; 367:1260-1263. A protomer of 2019-nCoV S in the RBD up conformation is shown (center) next to a protomer of SARS-CoV S in the RBD up conformation (right), displayed as ribbons and colored white (PDB ID: 6CRZ). (B) RBDs of 2019-nCoV and SARS-CoV aligned based on the position of the adjacent NTD from the neighboring protomer. The 2019-nCoV RBD is colored green and the SARS-CoV RBD is colored white. The 2019-nCoV NTD is colored blue. (C) Structural domains from 2019-nCoV S have been aligned to their counterparts from SARS-CoV S as follows: NTD (top left), RBD (top right), SD1 and SD2 (bottom left), and S2 (bottom right).

FIG. 20 is a graph showing 2019-nCoV S binds human ACE2 with high affinity adapted from Daniel Wrapp et al. Science 2020; 367:1260-1263. Surface plasmon resonance sensorgram shows the binding kinetics for human ACE2 and immobilized 2019-nCoV S. Data are shown as black lines, and the best fit of the data to a 1:1 binding model is shown in red.

FIG. 21 shows the antigenicity of the 2019-nCoV RBD adapted from Daniel Wrapp et al. Science 2020; 367:1260-1263. (A) SARS-CoV RBD shown as a white molecular surface (PDB ID: 2AJF), with residues that vary in the 2019-nCoV RBD colored red. The ACE2-binding site is outlined with a black dashed line. (B) Biolayer interferometry sensorgram showing binding to ACE2 by the 2019-nCoV RBD-SD1. Binding data are shown as a black line, and the best fit of the data to a 1:1 binding model is shown in red. (C) Biolayer interferometry to measure cross-reactivity of the SARS-CoV RBD-directed antibodies S230, m396, and 80R. Sensor tips with immobilized antibodies were dipped into wells containing 2019-nCoV RBD-SD1, and the resulting data are shown as a black line.

FIG. 22 shows tables of the input and output phage numbers during the panning process conducted to identify the SARS-CoV2 antibodies described herein.

FIG. 23 shows a table of the screening process conducted to identify the SARS-CoV2 antibodies described herein. SARS2 was screened via ELISA.

FIG. 24 is a binding curve showing SARS-RBD-Fc binding.

FIG. 25 outlines the Panning plan.

FIG. 26 is a graph showing virus infection. GD03 SARS and SARS2 pseudovirus was generated by transfecting LentiX-293T cells. ACE2+ target cells were incubated with varying dilutions of the pseudovirus supernatant for 48 hours before cell lysis and luciferase detection. The SARS2 pseudovirus displays decreased infection compared to the GD03 SARS strain which can be explained by low production titers or decreased viral entry into the target cells. However, the values for SARS2 are above baseline and can be used for introductory pseudovirus neutralization assays.

FIG. 27 shows optimization of the pseudovirus. FIG. 27A is a blot of Lentivirus Display showing Stable Expression of Human Antibodies on the Surface of Human Cells and Virus Particles (adapted from Taube R, Zhu Q, Xu C, Diaz-Griffero F, Sui J, et al. (2008). PLOS ONE 3(9): e3181) FIG. 27B is a bar graph adapted from Hoffmann et al., (Cell (2020), https://doi.org/10.1016/j.cell.2020.02.052) showing SARS-CoV-2 Cell Entry Depends on ACE2 and TMPRSS2.

FIG. 28 is a graph showing SARS/SARS-CoV2 pseudovirus infection of 293T cells transduced with ACE2. Two SARS-CoV2 spike pseudovirus constructs were used, WT spike and one with the end of the intraviron domain replaced with a gp41 tail. Two preps of pseudovirus were also used, one made in 150 mm plates with 3 day transfection, the second done in 100 mm plates with 2 day incubation (cells were floating after 2 days). Transfected with Lipofectamine 3000. 10,000 transduced 293T-ACE2 cells were cultured O/N. The next day pseudovirus supernatant was added to the sample in serial 2× dilutions, starting with straight supernatant in the top well. Plates were incubated for 48 hours before the supernatant was removed and cells were lysed with Promega passive lysis buffer. After equilibration period, Promega BioGlow luciferase reagent was added and the plate was read. Interval time was 0.5 sec, and the gain adjustment was at 40%.

FIG. 29 shows a table of the germline assignments for the first set of SARS-CoV2 antibodies identified.

FIG. 30 shows V gene germline sequence alignments of SARS-CoV2 antibodies identified as assigned by IMGT. Disagreements from the V gene germline sequence are highlighted in red.

FIG. 31 shows a table of the binding affinities for the first set of SARS-CoV2 antibodies identified. KD values measured on Octet with SA (streptavidin) sensors. Abs are scFv-Fc format except for CR3022. Sensors were coated with 2.5 ug/ml Biotinylated SARS-CoV-2 S1 protein (ACRO, S1N-C82E8). Abs were run at 3 concentrations, 25-12.5-6.25 nM and the kinetic parameters were calculated by linking the three curves. Control sensor was coated with biotinylated PD1 and 25 nM antibody was allowed to bind

FIG. 32 shows binding sensorgrams of the first set of SARS-CoV2 antibodies identified.

FIG. 33 outlines the competition assay protocol used for the first set of SARS-CoV2 antibodies identified.

FIG. 34 shows a graph of a saturation test. SA sensor loaded with S1-biotin (2.5 ug/ml, ACRO). Sensors were then dipped into wells containing a 250 nM ab solution and allowed to bind for 10 minutes. Following a short baseline in PBST, sensors were returned to the ab well to see if there was further binding. As demonstrated here, return to the ab well does not lead to additional binding, indicating that the antibodies are saturating the receptors at 250 nM.

FIG. 35 shows competition sensorgrams of the first set of SARS-CoV2 antibodies identified. Only the baseline followed by 2nd antibody step is shown here. Each sensor is saturated with an antibody (sensor key provided herein) and after a short baseline is added to wells containing the 2nd competing antibody. The antibody listed on each graph is the competing antibody. The light green lines are sensors loaded with S1, but no 1st antibody (shows maximal binding). Each set also has a “self” competition control, i.e. in Ab 7, sensors are first saturated with the Abs listed in the key at the bottom. The pink line is the competition of a sensor saturated with 250 nM Ab 7, followed by competition in a well with 125 nM Ab 7 (competition control). Based on these results, Ab 7 and 12 can fall into one bin and Ab2-2, 2-7, 2-10 can fall into the epitope recognized by CR3022.

FIG. 36 shows a table of the competition matrix. In the matrix, the names along the left side of the table are the 1st antibody, while the names across the tope are the 2nd/competing antibody. Boxes highlighted in red are considered blocking.

FIG. 37 shows a graph of ACE2 competition. Competition was conducted with ACE2; however protein quantity was limited and not a high enough concentration was used (only used ˜85 nM). No antibody control shows maximal ACE2 binding to S1 loaded sensors. The red line below that is CR3022, which is not reported to block ACE2 binding. The antibodies are below the CR3022 line with Ab 12, Ab 2-7, and Ab 2-10 being particularly flat.

FIG. 38 is a table of germline assignments for additional SARS-CoV2 antibodies identified.

FIG. 39 shows germline sequence alignments of additional SARS-CoV2 antibodies identified.

FIG. 40 is a table of the germline references for the additional SARS-CoV2 antibodies identified.

FIG. 41 is a table of the kinetics determined for the additional SARS-CoV2 antibodies identified. A couple of the antibodies bind RBD but not S1. Without wishing to be bound by theory, the differences in binding can be between the ACRO protein and Sino protein. Panning was done with proteins purchased from Sino Biologics. Some antibodies show increased binding to RBD compared to S1 (e.g. Ab 15 and Ab 25). RBD is also smaller than S1, so loading more molecules when doing RBD loading can lead to increased signal.

FIG. 42 is a table of a competition matrix. These studies were conducted in two separate assays, SARS-CoV2 Abs 13 thru 20 were run together and SARS-CoV2 Abs 21 thru 28 were another group. Ab 2-2 was used as a surrogate for CR3022 in both assays. The 1st ab was used at 250 nM (vertical axis) and the 2nd ab was used at 125 nM (horizontal axis). Green shaded boxes are non-competing pairs and red shaded boxes are competing pairs. Shading was done manually since our antibodies have a range of binding characteristics and maximum, unblocked binding can be below the threshold.

FIG. 43 shows a schematic of an epitope binning matrix for SARS-CoV2 antibodies.

FIG. 44 outlines the master competition with the groups for SARS-CoV2 antibodies.

FIG. 45 shows a schematic showing a table for the master binning for SARS-CoV2 antibodies 1 thru 28. Using data from the previous competition assays, a final competition assay with the 8 antibodies thought to be in separate bins was performed. Green shaded boxes are non-competing pairs, red shaded boxes are competing pairs, and the lighter green are debatable. Shading was done manually since the antibodies have a range of binding characteristics and maximum, unblocked binding can be below the threshold. The master bins clarified that Ab 28 and Ab 2-10 are both competing for the same epitope. Other antibodies showed interesting binning characteristics. For example, Ab 12 blocks Ab 14 and Ab 19, but Ab 14 does not block Ab 19 in either direction. A more detailed epitope mapping with finer resolution will be conducted to parse out these differences.

FIG. 46 is a graph showing SARS-CoV2 pseudovirus neutralization by anti-SARS-CoV2 scFv-Fcs. 293T-ACE2 cells were used as targets for SARS-CoV-2 pseudovirus. For neutralization, scFv-Fc was mixed with pseudovirus and incubated at RT for 1 hour. scFv-Fcs were used at about 25 nM* and pseudovirus was diluted 2×. After mixing pseudovirus/ab with the cells, the plates were incubated at 37° C. for 48 hours. Cells were then lysed with Promega passive lysis buffer and Promega bioglo luciferase was added.

FIG. 47 shows antibody nucleotide sequences for SARS-CoV-2 antibodies.

FIG. 48 shows antibody amino acid sequences for SARS-CoV-2 antibodies.

FIG. 49 shows a schematic of a human antibody discovery through pathogenic CoV Outbreaks of SARS, MERS and SARS2.

FIG. 50 shows a schematic of the size and genetic complexity of the Mehta I & II Human scFv-Phage Display Libraries.

FIG. 51 shows ribbon diagrams for Structural Basis of Neutralization and In Vivo Protection by 80R Antibody.

FIG. 52 shows Mutant MERS-CoVs were assigned to three epitope groups. Four escape mutants were chosen for cross neutralization assay.

FIG. 53 shows kinetic analysis of selected scFv-Fc candidates from SARS-2 S1/RBD panning. Three rounds of panning for anti-SARS-2 S1/RBD antibodies was done using recombinantly expressed soluble protein. a large number of antibodies with varying kinetic properties. Antibodies highlighted in blue are suspected of binding S1 outside of the RBD.

FIG. 54 shows Epitope binning of anti-SARS-CoV Spike scFvFc's. Competitive binding assay was run to identify antibodies that bind different epitopes. Sensors were first saturated with Ab 1 (250 nM), then Ab 2 (125 nM) was added. If there was additional antibody binding as demonstrated in the top panel, the antibodies were considered to bind separate epitopes. Results from these competition assays were compiled in a matrix as seen in the middle panel. Once the antibodies were grouped into general clusters, a more detailed competition assay was performed to further differentiate the broader bins as seen in Bin 3. Based on this analysis, the antibodies fell into 3 major bins and 7 minor bins.

FIG. 55 is a graph showing Percent Pseudovirus Neutralization by Anti-Spike scFvFcs from Different Bins.

FIG. 56 is a graph showing FACS Staining of Anti-Spike scFvFc to SARS2 Spike-293T cells. 100 k 293T+/−SARS2 Spike cells were stained with 100 ul of scFv-Fc at 5 μg/ml. Binding was detected by anti-human Fc APC. CR3022 is full IgG. This is selected data from FIG. 74.

FIG. 57 is a graph showing a dose-response curve for monoclonal antibody Ab-12 neutralization activity against live SARS-CoV-2 virus.

FIG. 58 is a graph showing a dose-response curve for monoclonal antibody Ab-27 neutralization activity against live SARS-CoV-2 virus.

FIG. 59 is a graph showing a dose-response curve for monoclonal antibody Ab-14 neutralization activity against live SARS-CoV-2 virus.

FIG. 60 is a graph showing a dose-response curve for monoclonal antibody Ab-19 neutralization activity against live SARS-CoV-2 virus.

FIG. 61 is a graph showing a dose-response curve for monoclonal antibody Ab-28 neutralization activity against live SARS-CoV-2 virus.

FIG. 62 is a bar graph showing pseudovirus neutralization by anti-SARS-CoV2 scFv-Fcs at 100 μg/ml. The dotted line approximates virus only and non-SARS-CoV-2 scFv-Fc neutralization. Values below the dotted line correspond to neutralization.

FIG. 63 is a bar graph for pseudovirus neutralization of anti-SARS-CoV2 scFv-Fc dilutions.

FIG. 64 is a bar graph for pseudovirus neutralization dilution curves for anti-SARS-CoV2 scFv-FCs. Ab 14=Ab 27>Ab 19>Ab 23>Ab 26>Ab 28

FIG. 65 is a table showing % blockade in a competition assay for master clones of Abs 1-28 via BLI (Octet).

FIG. 66 is a schematic showing Ab 1-28 master clone ACE2 competition. The value in the box is the percent binding normalized to the unblocked sensor. Shading was done manually since our antibodies have a range of binding characteristics and maximum, unblocked binding can be below the threshold.

FIG. 67 is a bar graph for pseudovirus neutralization of anti-SARS-CoV2 scFv-Fcs.

FIG. 68 is a line graph for pseudovirus neutralization of anti-SARS-CoV2 scFv-Fcs.

FIG. 69 is an epitope binning schematic. Based on competition matrix, Abs fell into 3 major bins which were further divided into 8 subbins. Kinetic measurements against S1 are below

FIG. 70 is a schematic of epitope binning/competition assay of Abs 29-40, repeat Abs 1-8.

FIG. 71 is a schematic of epitope binning of further competition with Ab 12 group. Competition of the antibodies in the Ab 12 group leads to some interesting sub bins. Additionally, a few of the strong binders compete with Ab 12, but do not compete with ab 27 or ACE2 (i.e. Ab 35). Based upon the results here, Ab 27 competition is more correlated with ACE2 blockade compared to Ab 12 competition.

FIG. 72 is a schematic of epitope binning of further competition with CR3022 group. Abs in the CR3022 bind have similar competition patterns. The only difference is that our Abs appear to block ACE2 whereas CR3022 does not. CR3022 is known to bind outside of the ACE2/RBD interface.

FIG. 73 is a schematic of epitope binning of further competition with S1 binding group. Abs 5, 23, 30 bind to the S1 outside of RBD. Interestingly the abs appear to target the same epitope as they compete with each other. To confirm, they do not compete with ACE2, CR3022, Ab 12, or Ab 27.

FIG. 74 is a plot depicting FACS binding of scFv-Fcs to 293T+/−SARS2 spike expressing cells. FACS binding at single concentration (5 ug/ml) of scFv-Fc with transduced 293T-SARS2-Spike expressing cells. Cells were first gated for BFP (transduced cells) and then for antibody binding. Some of the background can be due to the inherent stickiness of scFv-Fcs. Binding was detected with anti-human-Fc APC from Biolegend.

FIG. 75 is a binding curve showing different formats of Ab-12 binding to SARS-2 spike expressing cells. 293T cells were transduced with SARS-2 lentivirus. FACS was done with cells before sorting. Only BFP+ cells were used in the analysis of Ab binding.

FIG. 76 is a binding curve showing different formats Ab-12 binding to 293T cells. Untransduced 293T cells were used as the negative. IgG and scFv-Fc were detected by anti-human-Fc-APC and the Fab was detected by anti-His APC.

FIG. 77 is a schematic of an overview of bispecific antibodies and antibody-based approaches to SARS-CoV-2. The schematic is adapted from Kontermann et al., 2015.

FIG. 78 is a schematic of clinical applications of bispecific antibodies, adapted from Labrijn et al., 2019.

FIG. 79 is a schematic showing bispecific antibodies in the clinical pipeline adapted from Labrijn et al., 2019. * Withdrawn from market in 2017 for commercial reasons.

FIG. 80 is a schematic of SARS-CoV-2 Structural Features adapted from Wrapp et al., 2020.

FIG. 81 is a schematic showing the visualization of Epitopes on RBD Monomer (see, for B38: Wu et al., 2020; for CR3022: Yuan et al., 2020; for 5309: Pinto et al., 2020; and for P2B-2F6: Ju et al., 2020). PDB ID: 6MOJ.

FIG. 82 is a schematic showing the visualization of Epitopes on S Trimer (1 RBD “Up”). PDB ID: 6VSB.

FIG. 83 is a schematic showing the visualization of Epitopes on S Trimer (Closed State). PDB ID: 6X2C.

FIG. 84 is a schematic showing the conformational States of S Trimer. For example, the fusion-peptide proximal regions (FPPRs) can clamp down the three RBDs in the prefusion S trimer. However, one can occasionally flip out of position due to intrinsic protein dynamics, which allows RBDs to sample the up conformation” (Cai et al., 2020).

FIG. 85 is schematic showing a strategy to develop a bispecific antibody targeting ACE2 and CR3022 epitopes to fix RBD in the “up” position, thus exposing the S2′ cleavage site and facilitating irreversible S protein transition into more stable postfusion conformation. PDB ID: 6VSB.

FIG. 86 is a schematic showing a strategy to engineer bispecific antibodies that bind to distinct, non-overlapping epitopes on the S protein RBD. PDB ID: 6VSB.

FIG. 87 is a schematic showing a strategy to demonstrate enhanced binding affinities of bispecific antibodies to RBD epitopes. PDB ID: 6VSB.

FIG. 88 is a schematic showing a strategy to determine neutralization potential of bispecific antibodies towards SARS-CoV-2. PDB ID: 6VSB.

FIG. 89 is a schematic of a bispecific antibody engineering approach to target RBD epitopes on different protomers of same S trimer. PDB ID: 6VSB.

FIG. 90 is a schematic of a bispecific antibody engineering approach to target RBD epitopes on same protomer of different S trimers. Adapted from Neuman et al., 2006. PDB ID: 6VSB.

FIG. 91 is a schematic of a construct for engineering a tetravalent, Bispecific IgG1-scFv Heavy Chain Fusion that recognizes the ACE-2 binding epitope and the CR3022 epitope. Note: scFv is ˜35 Å with a (G₄S)₃ linker (Klein et al., 2009).

FIG. 92 is a linear schematic of a construct for engineering a tetravalent, Bispecific IgG1-scFv Heavy Chain Fusion that recognizes the ACE-2 epitope and the CR3022 epitope.

FIG. 93 is a schematic of a plasmid map for a construct for engineering a tetravalent, Bispecific IgG1-scFv Heavy Chain Fusion that recognizes the ACE-2 epitope and the CR3022 epitope.

FIG. 94 is a schematic of a construct for engineering a tetravalent, Bispecific IgG1-scFv Light Chain Fusion that recognizes the ACE-2 epitope and the CR3022 epitope.

FIG. 95 is a linear schematic of a construct for engineering a tetravalent, Bispecific IgG1-scFv Light Chain Fusion that recognizes the ACE-2 epitope and the CR3022 epitope.

FIG. 96 is a schematic of a plasmid map for a construct for engineering a tetravalent, Bispecific IgG1-scFv Light Chain Fusion that recognizes the ACE-2 epitope and the CR3022 epitope.

FIG. 97 is a schematic of a construct for engineering a tetravalent, Bispecific scFv Fusion that recognizes the ACE-2 epitope and the CR3022 epitope.

FIG. 98 is a linear schematic of a construct for engineering a tetravalent, Bispecific scFv Fusion that recognizes the ACE-2 epitope and the CR3022 epitope.

FIG. 99 is a schematic of a construct for engineering a tetravalent, Bispecific tandem scFv Fusion that recognizes the ACE-2 epitope and the CR3022 epitope.

FIG. 100 is a linear schematic of a construct for engineering a tetravalent, Bispecific tandem scFv Fusion that recognizes the ACE-2 epitope and the CR3022 epitope.

FIG. 101 is a schematic of a plasmid map for a construct for engineering a tetravalent, bispecific tandem scFv Fusion that recognizes the ACE-2 epitope and the CR3022 epitope.

FIG. 102 is a schematic of “Knob in Hole” Heterodimerization. Adapted from Sasorith et al., 2013.

FIG. 103 is a schematic of a design for “Knob in Hole” Constructs.

FIG. 104 is a schematic of a construct for engineering a bivalent, Bispecific Minibody (“Knob in Hole”) that recognizes the ACE-2 epitope and the CR3022 epitope.

FIG. 105 is a linear schematic of a construct for engineering a bivalent, Bispecific Minibody (“Knob in Hole”) that recognizes the ACE-2 epitope and the CR3022 epitope.

FIG. 106 shows a schematic of a plasmid map for a construct for engineering a bivalent, Bispecific Minibody (“Knob in Hole”) that recognizes the ACE-2 epitope and the CR3022 epitope.

FIG. 107 is a schematic of a design for “Knob in Hole” Constructs: KiH Construct 1.

FIG. 108 is a schematic of a design for “Knob in Hole” Constructs: KiH Construct 2.

FIG. 109 is a schematic of a design for “Knob in Hole” Constructs: KiH Construct 3.

FIG. 110 is an image of an SDS-PAGE gel. The four constructs were expressed and are the correct size in their native and reduced forms. Parental Ab 12 IgG and Ab 2-7 scFv-Fc were run as controls. Reduced (+10% BME) and non-reduced samples were run.

FIG. 111 is an Octet BLI binding curve for kinetic measurements for the bispecific antibodies.

FIG. 112 is schematic showing antibody binding kinetics as determined by BLI using SA sensors and biotinylated RBD.

FIG. 113 is a binding sensorgram of the interaction between biotinylated human FcRn and antibodies measured with an Octet platform. Human FcRn at 5 μg/mL was immobilized as the ligand onto Streptavidin biosensors, and antibodies were used as analytes at 500 nM. Kinetic measurements for FcRn binding are in table below.

FIG. 114 is a graph showing SEC characterization of indicated bispecific antibodies.

FIG. 115 are graphs showing FACS binding curves for aSARS-CoV2 bispecific antibodies against 293T cells stabley expressing the SARS-CoV-2 spike.

FIG. 116 are graphs showing GeoMFI curves from FACS binding experiments for aSARS-CoV2 bispecific antibodies.

FIG. 117 are graphs showing binding curves for aSARS-CoV2 bispecific antibodies to negative cells.

FIG. 118 shows graphs of FACS analyses of stably transduced 293T-SARS2 spike cells that were stained with various concentration of anti-SARS-2 antibody, followed by a constant concentration of soluble, biotinylated RBD. Antibody binding to the cell was detected with anti-human Fc-PE and soluble RBD binding to free antibody arms was measured with streptavidin APC. It was determined if all arms of the bispecific are able to bind on the spike or if there are available free arms. FIG. 118A depicts where all binding regions of Ab are occupied (e.g., no soluble RBD binding). FIG. 118B depicts where only some binding regions of Ab are occupied (e.g., soluble RBD binding).

FIG. 119 shows graphs of FACS analyses of IgG Fusions (2 nM). Ab 12 IgG is not able to bind free RBD in solution (all binding arms are occupied by cell surface Spike). The aPD1 LC fusion maintains this binding configuration, however aPD1 HC fusion is not able to get both binding arms to bind the cell simultaneously. With the PD1 antibody, Ab 2-7 as a HC fusion is able to bind the cells and soluble RBD. The Ab 12-Ab 2-7 LC fusion is able to bind more molecules per cell (higher MFI compared the HC fusion and parental IgG).

FIG. 120 shows graphs of FACS analyses of tandem scFv-Fcs (2 nM). Ab 12 scFv-Fc does not bind as well soluble RBD when bound to the cell surface. The addition of the second scFv-Fc forces the tandem scFv-Fc to adopt a confirmation that has the second arm more accessible to soluble RBD. The Ab 12/Ab 2-7 tandem shows increased binding to the cell surface (increased MFI compared to Ab 12 scFv-Fc). Ab 12/Ab 2-7 has increased binding, however it also has increased free arms to bind soluble RBD compared to Ab2-7/Ab 12 Tandem.

FIG. 121 is graph showing live SARS-2 virus neutralization with aSARS-CoV2 bispecific antibodies (luciferase assay).

FIG. 122 is a graph showing live SARS-2 virus neutralization with aSARS-CoV2 Ab12 IgG and IgG fusion antibodies (luciferase assay).

FIG. 123 is a DSC plot of the SYPRO orange fluorescence relative temperature showing thermal stability of aSARS-CoV2 bispecific antibodies.

FIG. 124 is a DSC plot of the d(RLU)/dT.

FIG. 125 is a schematic of Knob in Hole (KiH) designs. Knob in hole designs were generated with a tandem scFv-Fc on one side and a mono scFv-Fc on the other. Heterodimerization can lead to a trivalent antibody that targets 3 epitopes on the S1 domain of the spike. KiH designs rely on steric clashes between different side chains to create biased dimerization potential between the monomers. Asymmetric cysteines were added to some of the constructs to improve dimerization.

FIG. 126 is a schematic of the KiH Construct 1.

FIG. 127 is a schematic of the KiH Construct 2.

FIG. 128 is a schematic of the KiH Construct 3.

FIG. 129A is a graph of size-exclusion chromatography (SEC) traces of select multi-specific antibodies and a parental monoclonal antibody. Absorbance was measured at a wavelength of 280 nm

FIG. 129B is an image of a gel of SDS-PAGE analyses of trispecific antibodies under normal and reducing (2-mercaptoethanol) conditions. From left to right: Ab12-Ab2-7 Tandem scFv-Fc Knob-Ab5 scFv-Fc Hole SS Fusion, Ab12-Ab2-7 Tandem scFv-Fc Knob-Ab5 scFv-Fc Hole ZW1 Fusion, Ab12-Ab2-7 Tandem scFv-Fc Knob-Ab5 scFv-Fc Hole LT Fusion.

FIG. 130 is a schematic of the Ab12/Ab2-7 tandem scFv-Fc construct showing the nucleic acid and amino acid sequences.

FIG. 131 is a schematic of the Ab2-7/Ab12 tandem scFv-Fc construct showing the nucleic acid and amino acid sequences.

FIG. 132 is a schematic of the Ab5 scFv-Fc construct showing the nucleic acid and amino acid sequences.

FIG. 133 is a schematic of the LT-knob-T22Y Y5C-6×His construct showing the nucleic acid and amino acid sequences.

FIG. 134 is a schematic of the LT-hole-Y86T E13C-FLAG tag construct showing the nucleic acid and amino acid sequences.

FIG. 135 is a schematic of the LT-hole-Y86T E13C-C9 tag construct showing the nucleic acid and amino acid sequences.

FIG. 136 is a schematic of the SS-knob-T22W S10C-6×His construct showing the nucleic acid and amino acid sequences.

FIG. 137 is a schematic of the SS-hole-T22S L24A Y86V Y5C-FLAG tag construct showing the nucleic acid and amino acid sequences.

FIG. 138 is a schematic of the SS-hole-T22S L24A Y86V Y5C-C9 tag construct showing the nucleic acid and amino acid sequences.

FIG. 139 is a schematic of the ZW1-knob-T6V T22L K79L T81W-6×His tag construct showing the nucleic acid and amino acid sequences.

FIG. 140 is a schematic of the ZW1-hole-T6V L7Y F85.1A Y86V-FLAG tag construct showing the nucleic acid and amino acid sequences.

FIG. 141 is a schematic of the ZW1-hole-T6V L7Y F85.1A Y86V-C9 tag construct showing the nucleic acid and amino acid sequences.

FIG. 142 are graphs of scFv-Fc neutralization studies of the live SARS-CoV-2 virus.

FIG. 143 is a graph of neutralization studies of the live SARS-CoV-2 virus with mAb12 as an IgG or scFv-Fc.

FIG. 144 is a graph of neutralization studies of the live SARS-CoV-2 virus with mAb14 as an IgG or scFv-Fc.

FIG. 145 is a graph of neutralization studies of the live SARS-CoV-2 virus with mAb27. Note: Ab 27 IgG was actually Ab 2-2 IgG, for 27 data see FIG. 146.

FIG. 146 is a graph of neutralization studies of the live SARS-CoV-2 virus with mAb27.

FIG. 147 is a graph of neutralization studies of the live SARS-CoV-2 virus with mAb29 as an IgG or scFv-Fc.

FIG. 148 is a graph of neutralization studies of the live SARS-CoV-2 virus with mAb2-7 as an IgG or scFv-Fc.

FIG. 149 is a graph of neutralization studies of the live SARS-CoV-2 virus with mAb38 as an IgG or scFv-Fc.

FIG. 150 is a graph of neutralization studies of the live SARS-CoV-2 virus with mAb5 as an IgG or scFv-Fc.

FIG. 151 is a graph of neutralization studies of the live SARS-CoV-2 virus with PD-1 control as an IgG or scFv-Fc.

FIG. 152 shows graphs of neutralization studies of the live SARS-CoV-2 virus engineered to express luciferase. Neutralization was also tested with a mouse adapted variant of SARS-CoV-2 (Dinnon, K. H., Leist, S. R., Schafer, A. et al. A mouse-adapted model of SARS-CoV-2 to test COVID-19 countermeasures. Nature 586, 560-566 (2020). https://doi-org.ezp-prodl.hul.harvard.edu/10.1038/s41586-020-2708-8). Mixtures of various scFv-Fcs were tested: Ab 12+Ab 2-7 (mix 1), Ab 27+Ab 2-7 (mix 2), Ab 12+Ab 27 (mix 3), Ab 27+Ab 35 (mix 4), Ab 27+Ab 2-7+Ab 30 (mix 5).

FIG. 153 shows graphs of neutralization studies of the live SARS-CoV-2 virus engineered to express luciferase.

FIG. 154 shows graphs of neutralization studies of the live SARS-CoV-2 virus comparing WT and D614G mutants using virus engineered to express luciferase.

FIG. 155 shows graphs of weight loss in hamsters (TOP) and Viral load of lung tissues, 3 dpi, PFU/g (BOTTOM) of hamsters. Therapeutic treatment of Syrian golden hamsters post infection with Ab 12 IgG or Ab 2-7 scFv-Fc leads to a 513.9- and 5-fold reduction respectively compared to control treated animals. No significant difference in weight loss is observed.

FIG. 156 shows graphs of serum neutralization, day 3 post infection in hamsters. Serum was collected 3 days post infection and tested in vitro neutralization assays. Serum from Ab 12 treated animals is able to neutralize virus, whereas serum from Ab 2-7 and control treated animals is not.

FIG. 157 shows images of lung pathology studies and a graph depicting gross lesions Score, 3 dpi.

FIG. 158 shows images of lung pathology studies and a graph depicting gross lesions score, 3 dpi.

FIG. 159 are graphs showing lung lesion scores in hamsters treated with mAb 12.

FIG. 160 shows a graph a of serum neutralization study.

FIG. 161 are graphs showing lung lesion scores in hamsters treated with mAb 12.

FIG. 162 are fluorescent micrographs showing the visualization of Ab 12 IgG uptake by THP1 cells via Fc receptors. This is a surrogate for ADE infection.

FIG. 163 shows competition of the antibodies in the Ab 12 group. For example, a few of the strong binders compete with Ab 12, but do not compete with Ab 27 or ACE2 (i.e. Ab 35). Ab 27 competition is more correlated with ACE2 blockade compared to Ab 12 competition.

FIG. 164 shows further competition within CR3022 group. Abs in the CR3022 bind have similar competition patterns. The only difference is that the Abs appear to block ACE2 whereas CR3022 does not. CR3022 is known to bind outside of the ACE2/RBD interface.

FIG. 165 shows further competition within S1 binding group. Abs 5, 23, 30 bind to the S1 outside of RBD. Without wishing to be bound by theory, the antibodies target the same epitope as they compete with each other. To confirm, they do not compete with ACE2, CR3022, Ab 12, or Ab 27.

FIG. 166 is a schematic of epitope binning. Bin 1: S1, non RBD binding; Bin 2: RBD binding, competes with CR3022; Bin 3: RBD binding, non CR3022 competition.

FIG. 167 is a graph showing SARS-CoV-2 virus neutralization by scFv-Fc in a PRNT assay.

FIG. 168 shows graphs of IgG vs scFv-Fc virus neutralization in PRNT. scFv-Fcs and IgGs were tested in parallel SARS-CoV-2 neutralization assays. Abs 12 and 38 showed minimal loss in neutralization efficacy, however Abs 14, 27, 29, and 2-7 displayed substantial loss. Of the 3 most potent scFv-Fcs, Ab 12 is an Ab that maintains its ability to neutralize as an IgG, while the IC50 for Ab 14 and Ab 27 shifts the right by 40- and 950-fold, respectively.

FIG. 169 shows FACS binding curves with 293T-Spike cells show a pronounced decrease in binding for Abs 14, 27, and 2-7 in IgG format, whereas Ab 12 shows an increase in binding. Kinetic measurements via BLI of selected Abs show that conversion from scFv to IgG does not have a significant effect on the KD values for these antibodies when binding.

FIG. 170 shows graphs of pathology scores for animals treated with PBS or anti-SARS-CoV2 Abs 12 or 2-7. *=p<0.05; ns=Not significant (Kruskall-Wallis test with Dunn's post-hoc correction)

FIG. 171 shows lung histology images. A) and B) are not depicted in this image. C) Control lung. Consolidation with multiple foci of inflammatory infiltration. Magnified images (locations on low magnification images marked with numbers): (1) Airways are obstructed by inflammatory cells (combination of MNC and PMNs). (2) Airway epithelial hyperplasia notable. Perivascular cuffing and congestion prominent. D) Ab 2-7 lung. Consolidation with multiple foci of inflammatory infiltration. (1) Pleuritis noted, but less severe. (2) Fewer inflammatory cells in airways, but hyperplasia of airway epithelia is still prominent. E) Ab 12 lung. Consolidation markedly reduced, with fewer and smaller foci of inflammatory infiltration. Infiltrating inflammatory cells present in some airways. (1) Pleuritis is moderate relative to control. (2) Airway epithelial hypertrophy still present.

FIG. 172 is a graph showing Spike shedding induced by scFv-Fcs. FACS based Spike shedding experiment comparing parental Abs with the BsAbs. A decrease in median fluorescence correlates to an increase in spike shedding whereas an increase in fluorescence indicates minimal shedding is observed. Antibodies that are grouped into a similar sub-bin show similar levels of shedding. Western blot image is also shown depicting the detection shed S1 in supernatant from Ab 12 IgG spike shedding experiment confirming decreasing fluorescence is a result of shedding and not internalization of the spike-Ab complex.

FIG. 173 is a cryo-EM image of Ab 5, starting at medium resolution. Without wishing to be bound by theory, Ab 5 is an anti-NTD binder.

FIG. 174 are cryo-EM images of Ab 38: 2D classification.

FIG. 175 shows cryo-EM images of Ab 12, at Medium resolution (5 Å) to begin. Without wishing to be by theory, the red arrow in the bottom figure points to a quaternary epitope: Glycan N165 from a different monomer can be involved in the epitope.

FIG. 176 shows a map refined to a nominal resolution of 2.97 Ångstroms.

FIG. 177 shows a schematic of the refinement of a mixed population.

FIG. 178 shows images of cryoEM of the scFv-bound species and the map in the region of the RBD/scFv.

FIG. 179 shows images of a cryoEM map depicting three scFv molecules bound to a spike trimer, with 3-fold symmetry.

FIG. 180 shows images of a cryoEM map depicting a mixed interaction between heavy chain and light chain.

FIG. 181 shows cryoEM images of a further refined Ab2-7. Spike is blue, heavy chain is orange, light chain is gray.

FIG. 182 shows broad epitope binding for whole cell panning derived phage. Phage supernatant of unique clones were tested via ELISA against the different SARS-CoV-2 subunits (S1, S2, RBD) and against full length spikes from SARS-CoV-2 (D614G) and SARS-CoV. IL2-Fc was used as a negative control. Values shown are OD450 values, phage binding is detected with anti-M13-HRP. As shown here, a number of phage bind to the full length spike but not to any of the individually expressed domains. Without wishing to be bound by theory, this can be due to a conformational shift or junction epitope. These clones do not appear to non-specifically bind to the plates as the IL2 signal is negligible. One antibody appears to cross react between SARS1 and SARS2 and it binds the S2 domain, which is more conserved than the S1 domains.

FIG. 183 is a photograph of an SDS gel (4-12% Bolt Gel ran in MES) with aSARS2 trispecific (Knob in Holes) expressed and purified for animal studies. Lanes 2-7 are non reduced. Lanes 9-14 are reduced with 10% BME. Lane 12=aSARS-CoV-2 Ab 12. Lanes 2-7=aSARS-CoV-2 Ab 2-7. The names of the sample denote the KiH format (SS, LT, ZW) and the order of the tandem Abs on the knob side (12/2-7 or 2-7/12). The monomer side (hole) is Ab 5.

FIG. 184 is a table showing the kinetics for trispecific antibodies.

FIG. 185 is a graph showing neutralization data of engineered human monoclonal antibodies against SARS-CoV-2 (trispecific construct: LT Ab12/2-7).

FIG. 186 is a graph showing neutralization data of engineered human monoclonal antibodies against SARS-CoV-2 (trispecific construct: SS Ab12/2-7).

FIG. 187 is a graph showing neutralization data of engineered human monoclonal antibodies against SARS-CoV-2 (trispecific construct: ZW1 Ab12/2-7).

FIG. 188 is a graph showing neutralization data of engineered human monoclonal antibodies against SARS-CoV-2 (trispecific construct: LT Ab2-7/12).

FIG. 189 is a graph showing neutralization data of engineered human monoclonal antibodies against SARS-CoV-2 (trispecific construct: SS Ab2-7/12).

FIG. 190 is a graph showing neutralization data of engineered human monoclonal antibodies against SARS-CoV-2 (trispecific construct: ZW1 Ab2-7/12).

FIG. 191 is a graph showing neutralization data of engineered human monoclonal antibodies against SARS-CoV-2 (trispecific construct: Ab12/2-7 Tandem+Ab5).

FIG. 192 is a graph showing neutralization data of engineered human monoclonal antibodies against SARS-CoV-2 (trispecific construct: Ab2-7/12 Tandem+Ab5).

FIG. 193 is a graph showing neutralization data of engineered human monoclonal antibodies against SARS-CoV-2 (trispecific construct: Ab12+Ab2-7+Ab5 scFvFc).

FIG. 194 are graphs of spike shedding time courses relative to binding to T5. ACE2 reaches 50% of the original intensity. Ab 12, LC fusion, and Ab 12/Ab2-7 tan scFv-Fc have the greatest effect on spike shedding, though the tandem scFv-Fc leads to a slower rate of shedding. HC fusion is on par with ACE2.

FIG. 195 shows graphs of in vitro neutralization of SARS-CoV-2 virus in PRNT assay.

FIG. 196 shows a graph of live virus neutralization using virus engineered to express luciferase.

FIG. 197 shows a graph of live virus neutralization using virus engineered to express luciferase.

FIG. 198 shows a graph and table of a test for neutralizing activity of Ab12 and Ab2-7 constructs, prophylactic treatment in vivo with MOUSE ADAPTED SARS-CoV-2 virus. mice: 5 12-month old female Balb/c mice (Envigo) per treatment group; mAb treatment: 200 ug of each given i.p.; 12 hrs prior (prophyl) infection; infection: 10≡pfu mouse-adapted SARS-CoV-2 (SARS-2 MA) intranasally; readout: d2pi lung titer by plaque assay. Note: groups Ab12IgG and Ab12 scFv-Fc are n=4. Fold improvement=Mean aPD1 IgG/mean sample. Bottom chart shows lung titers after prophalyactic treatment of hACE2 transgenic mice using WT SARS-2 virus.

FIG. 199 shows an overall kinetics table.

FIG. 200 shows biochemical characterization of Ab 12 and 2-7 scFv-Fcs. FIG. 200A is a competition matrix with CR3022 and ACE2. FIG. 200B is a bar graph showing antibody cross binding that was measured by ELISA with Tor2 SARS-CoV spike. FIG. 200C is a graph showing PRNT neutralization assays performed with SARS-CoV-2 (isolate USA-WA1/2020) demonstrating that Ab 12 is the more potent of the two scFv-Fcs. Both IgG and scFv-Fc formats were tested in parallel PRNT neutralization assays. Ab 12 in FIG. 200D shows minimal change in neutralization efficacy between the two formats, however Ab 2-7 IgG in FIG. 200E displayed complete loss of neutralization. FIG. 200F is a FACS binding curve showing percent of cells positively labeled by antibodies. While Ab 12 IgG shows a shift to the left compared to the scFv-Fc, Ab 2-7 shows a ˜10-fold shift to the right. FIG. 200G is a graph of geometric mean fluorescence showing a more pronounced decrease in Ab 2-7 binding from the scFv-Fc to IgG.

FIG. 201 shows structural studies of Ab 2-7 and Ab 12 bound to SARS-CoV-2 spike. Cryo-EM structures were generated for Ab 2-7 scFv and Ab 12 Fab bound to full length SARS-CoV-2 spike. FIG. 201A is a Cryo-EM structure showing two Ab 2-7 scFvs bound to a SARS-CoV-2 spike trimer, with the Ab 2-7 heavy chain in red, light chain in pink, and the spikes in various shades of blue. FIG. 201B is a ribbon diagram depicting that Ab 2-7 binding rotates the RBD into the “up” confirmation seen with CR3022 (M. Yuan, et al., A highly conserved cryptic epitope in the receptor-binding domains of SARS-CoV-2 and SARS-CoV. Science 368, 630-633 (2020)). Image on the right is an overlay of unbound RBD in the “down” configuration (yellow) compared to Ab 2-7 bound RBD (blue). FIG. 201C is a schematic showing that the CH1 and CL domains of the Fab can sterically clash with a neighboring spike protein (circled in blue), providing a structural explanation for the lack of Ab 2-7 IgG binding and neutralization due to the angle of approach for Ab 2-7 scFv. FIG. 201D is a model without wishing to be bound by theory that if Ab 2-7 light chain makes the predominant contacts with the RBD, the distance between the C termini is 50 A (black line) while heavy chain dominance results in a distance of 115 A (purple line), which can be too long for an scFv-Fc to bridge. FIG. 201E shows a Cryo-EM map density for Ab 12 Fab bound to full length spike, with Fabs colored orange, monomers of the spike in green, blue and violet, and glycans in yellow. FIG. 201F is a ribbon model of two Ab 12 Fabs in complex with spike. Inset, the binding site of Ab 12 (orange) on the RBD (teal) overlaps with that of ACE2 (yellow). FIG. 201G shows the location of the Q498Y/P499T mutations in SARS-CoV-2 MA virus compared to the Ab12 epitope.

FIG. 202 shows therapeutic efficacy of Ab 12 IgG and Ab 2-7 scFv-Fc in Syrian golden hamster model As Ab 2-7 IgG does not neutralize in vitro, Ab 2-7 scFv-Fc and Ab 12 IgG were tested. FIG. 202A is a graph showing that therapeutic treatment of Syrian golden hamsters post infection with Ab 12 or Ab 2-7 leads to a 513.9- and 5.2-fold reduction of viral loads respectively compared to control (PBS) treated animals. FIG. 202B are graphs of pathology scores for animals treated with PBS, Ab 12 IgG, or Ab 2-7 scFv-Fc. Scores were determined based on the criteria in FIG. 217. FIG. 202C is a histological representative image of stained control lung. Consolidation with multiple foci of inflammatory infiltration. Magnified images (locations on low magnification images marked with numbers): (1) Airways are obstructed by inflammatory cells (combination of MNC and PMNs). (2) Airway epithelial hyperplasia notable. Perivascular cuffing and congestion prominent. FIG. 202D is a histological representative image of stained Ab 2-7 lung. Consolidation with multiple foci of inflammatory infiltration. (1) Pleuritis noted, but less severe. (2) Fewer inflammatory cells in airways, but hyperplasia of airway epithelia is still prominent. FIG. 202E is a histological representative image of stained Ab 12 lung. Consolidation markedly reduced, with fewer and smaller foci of inflammatory infiltration. Infiltrating inflammatory cells present in some airways. (1) Pleuritis is moderate relative to control. (2) Airway epithelial hypertrophy still present.

FIG. 203 shows the design and in vitro characterization of anti-SARS-CoV-2 BsAbs. FIG. 203A shows the design of the four anti-SARS-CoV-2 BsAbs. Constant regions are colored in gray, Ab 12 binding domains are blue, and Ab 2-7 binding domains are orange. In vitro neutralization was performed with the IgG fusion BsAbs (FIG. 203B) and tandem scFv-Fcs (FIG. 203C). The IC50 values and fold change of the IC50 relative the 1:1 mixtures are tabulated below their respective graphs, confirming that the BsAbs demonstrate improved neutralization compared to the parental Abs and mixtures (FIGS. 203D-E). CompuSyn was used to calculate the combination index (CI) for each therapy and extrapolated for various doses. CI values>1 indicates antagonistic interactions, values<1 indicate synergistic interactions, and values=1 indicate an additive effect. The IgG fusion BsAbs lead to greater synergy than a mixture of individual components while the order of scFvs in the tandem drastically altered the level of synergy achieved. FIG. 203F is a graph of a FACS based spike shedding experiment comparing parental Abs with the BsAbs. A decrease in median fluorescence correlates to an increase in spike shedding whereas an increase in fluorescence indicates minimal shedding is observed. FIG. 203G is an image of a Western blot detecting shed S1 in supernatant from Ab 12 IgG spike shedding experiment confirming decreasing fluorescence is a result of shedding and not internalization of the spike-Ab complex.

FIG. 204 shows the prophylactic efficacy against SARS-CoV-2 virus in mouse models. Two mutations in RBD were identified to allow the RBD to bind mouse ACE2. FIG. 204A shows that the mutations Q498T (red) and P499Y (yellow) are located towards the end of the RBD and on the edge of the ACE2 binding region. FIG. 204B is a graph that shows In vitro neutralization was performed with recombinant nLuc SARS-CoV-2 MA virus. Ab 12 scFv-Fc shows no difference in neutralization between WT and SARS-CoV-2 MA virus. Ab 12 IgG shows greater than a log shift to the right against the mouse adapted virus. FIG. 204C is a graph that shows Prophylactic efficacy of Ab 12 and Ab 2-7 mono- and bi-specific antibodies were tested in aged Balb/c mice. Mean PFU after infection is tabulated in the table to the right of the chart, with fold improvement relative the αPD1 negative control. FIG. 204D is a graph showing BsAb-HC fusion and scFv-Fc mixture were selected for prophylactic testing in transgenic hACE2 mice with WT SARS-CoV-2 virus. Both treatments lead to reduction of viral titers below the limit of detection in the samples except for one animal treated with 10 mg kg-1 of BsAb-HC that showed residual virus.

FIG. 205 shows the characterization and analysis of scFv-Fcs. FIG. 205A) Biolayer interferometry traces for Ab 12 (left) and 2-7 (right) scFv-Fcs. Abs were tested at 50, 25, and 12.5 nM against biotinylated RBD. The red trace shows the classical 1:1 binding fit model for the given data sets. FIG. 205B) Kinetic constants derived from the traces in FIG. 205A. FIG. 205C) kinetic measurements and IC50 with PRNT. FIG. 205D) Germline and allele assignments for variable domains of Ab 12 and Ab 2-7. Sequences for the V region of (FIG. 205E) Ab 12 and (FIG. 205F) Ab 2-7 were aligned with their native germline sequences with differences highlighted in red. Ab 12 HC is heavily mutated from the original IGHV4-59 sequences, whereas the LC shows minimal mutations outside of the FW1 region. Ab 2-7 has very few mutations compared to the germline sequences. Germline assignment and V gene alignment was performed by IMGT (Brochet, Lefranc and Giudicelli 2008).

FIG. 206 shows Antibody arm occupancy when binding to SARS-CoV-2 spike expressing cells by staining 293T-Spike cells with our antibodies followed by biotinylated RBD. Binding of the aSARS-CoV-2 Ab and RBD were detected by ahFc-PE and streptavidin-APC respectively. FIG. 206A) RBD-streptavidin-APC and FIG. 206B) ahFc-PE background binding to Spike expressing cells. FIG. 206C) Ab 12 IgG is not able to bind RBD in solution as shown by the lack of PE signal in the FACS plot, demonstrating that both arms are occupied by the cell surface spike proteins. FIG. 206D) Conversely, Ab 2-7 scFv-Fc is only able to bind the cell surface spike protein with one arm at a time as shown by the binding of RBD and the increase in APC signal. FIG. 206E) FACS based dual staining experiment with Ab 12 and Ab 2-7 scFv-Fc shown at different concentrations. Samples were incubated for 1 hour with soluble RBD at 4° C. before washing and the addition of secondary. At sub-saturating levels, Ab 12 scFv-Fc binds with both arms on the spike and is unable to bind soluble RBD. Ab 2-7 scFv-Fc is not able to bind with both arms on the spike as when Ab 2-7 is bound we are also able to bind soluble RBD. BLI was used to measure the kinetics of Ab 2-7 in scFv (FIG. 206F) and scFv-Fc (FIG. 206G) formats to biotinylated RBD. Kinetic values show in (FIG. 206H) demonstrate that Ab 2-7 is a high affinity antibody with low avidity dependency.

FIG. 207 shows dual binding of Ab 12 and Ab 2-7 scFv-Fcs Different concentrations of Abs were bound to 293T cells stably expressing the SARS-CoV-2 spike for 1 hour at 4° C., followed by the addition of soluble biotinylated RBD. Ab binding and RBD capture was detected by ahFc-PE and streptavidin-APC, respectively. Cells were incubated with RBD for different time periods (30, 60, 120 min) and at different temperatures (4° C., RT) to ensure that Abs and RBD were at equilibrium. At lower concentrations (˜2.5 nM), Ab 12 scFv-Fc stops binding soluble RBD, while still binding strongly to the cells. Ab 2-7 scFv-Fc shows binding to soluble RBD at concentrations with significant amounts of Ab bound to the cells.

FIG. 208 shows serum neutralization from Ab 12 IgG or Ab 2-7 scFv-Fc treated animals Ab 12 IgG remains active in the serum of infected animals for 3 days post infection and is able to neutralize virus in vitro. Ab 2-7 scFv-Fc treated animals do not have in vitro protective antibodies in the serum 3 days post infection.

FIG. 209 shows biochemical characterization of bispecific antibodies. FIG. 209A) SDS-PAGE gel of IgG fusions and tandem scFv-Fcs. The first four lanes are non-reduced samples and the last 4 lanes are reduced with 10% BME. FIG. 209B) Size exclusion chromatography of the bispecific antibodies showing peaks at the expected elution volume and minimal aggregation. FIG. 209C) Thermal stability of our mono and bispecific constructs measured by SYPRO Orange thermal shift assay. Graph on the left is the raw fluorescence vs temperature, graph on the right is the change of fluorescence vs temp. Melting peaks of composite antibodies is similar to that of the individual components, demonstrating that the fusions do not significantly affect the stability of the parental IgG or scFv. FIG. 209D) BLI curves for bispecific and monospecific Abs show strong binding the RBD. Ab 12 is the dominant binding moiety in these bispecifics and that is also shown in the binding curves here. FIG. 209E) Engineered BsAbs display similar kinetics to FcRn as parental antibodies, binding at pH 6 and disassociating at pH 7.4.

FIG. 210 shows mono and bispecific antibody competition via BLI. Streptavidin sensors were loaded with biotinylated RBD, saturated with Ab 1, and then competing Ab 2 was allowed to bind. FIG. 210A) Ab 12 and Ab 2-7 and their BsAbs were tested for cross-competition or blockade of ACE2 and CR3022. Red boxes show competition, blue boxes are no competition. BsAbs block target epitopes. FIG. 210B) Hybrid control BsAbs encoding only one anti-RBD Ab and a second control anti-PD1 IgG or scFv were tested in competition assay. Interestingly, both of the BsAb-HC fusion hybrids completely blocked both epitopes, demonstrating that the blockade in this case is steric (red boxes) and not due to direct competition.

FIG. 211 shows mono specific and BsAb competition via BLI. RBD coated sensors were saturated with each of 1st Abs listed on right and then tested for binding of each Ab to saturated sensor. FIGS. 211A-B), sensors saturated with Ab2-7 scFv-Fc are able to bind Ab 12 IgG or scFv-Fc but not BsAbs, whereas Ab2-7 scFv-Fc (FIG. 211C) or CR3022 IgG (FIG. 211D) are only able to bind Ab12 IgG or scFv but not BsAbs. ACE2 saturated sensors were not able to bind any mono or BsAbs (FIG. 211E). In the plots, PBST is used as the no competition control and shows maximal binding.

FIG. 212 shows control hybrid BsAbs were generated by replacing one pair of the Ab 12 IgG/scFv or Ab 2-7 scFv binding domains with a control anti-hPD1 IgG or scFv which were then used in competition assays to examine if competition was due to epitope sharing or steric hinderance. RBD-coated sensors were saturated with the six listed Abs (Ab1) followed by the same as competing Ab2. Unexpectedly, Ab 12 IgG-PD1 scFv HC fusion was able to block both Ab 2-7 scFv-Fc (FIG. 212C) and CR3022 IgG (FIG. 212D) while anti-hPD1 IgG-Ab 2-7 scFv HC fusion was able to block Ab 12 IgG (FIG. 212A) and scFv binding (FIG. 212B). Other BsAb-LC fusion and tandem BsAbs showed less off target inhibition. FIG. 212E) Bispecific are able to block ACE2. In the plots, PBST is used as the no competition control and shows maximal binding.

FIG. 213 shows FACS binding characteristics of anti-SARS-CoV-2 bispecific fusions. Dual binding is dose dependent at saturating concentrations, there are free arms. To accommodate this, dose response curves were performed, and a representative concentration was selected. FIGS. 213A-B) Similar to Ab 2-7 scFv-Fc, Ab 12-Ab 2-7 BsAb-HC and BsAb-LC fusions are not able to occupy the binding arms of the bispecific as shown by simultaneous binding of the bispecifics to the cells and to soluble RBD in solution. FIG. 213C) hybrid BsAb-HC fusion with Ab 12 IgG and non-specific scFv continues to show binding to free RBD in solution. FIG. 213D) Hybrid BsAb-LC fusion resembles that of the parental IgG and is able to bind both arms of the IgG to cell surface spike protein. FACS staining plots demonstrate that the expanded length of the BsAb-HC fusion is not able to fit into the available binding area to successfully bind both arms simultaneously. The BsAb-LC fusion, which is “wider” but maintains the same length is still able to occupy both arms on cell surface spike. Histograms representing the geoMFI for BsAb-HC fusion (FIG. 213E) and BsAb-LC fusion (FIG. 213F) binding to Spike cells demonstrate that the geoMFI is substantially higher with the BsAb-LC fusion compared to the BsAb-HC fusion, demonstrating greater Ab occupancy on the cell surface. Binding was performed as a dose response curve, starting at 10 nM and following a 4× dilution pattern. Tandem scFv-Fcs were also tested in the dual binding assay. FIG. 213G) Ab 12 scFv-Fc is able to bind the spike with both arms as no soluble RBD is bound. FIGS. 213H-I) Both tandem scFv-Fcs shows binding of both arms. FIGS. 213J-K) Hybrid tandems with Ab 12/aPD1 scFv returns to the binding pattern of the parental Ab 12 scFv-Fc, demonstrating that the tandem does not interfere with the ability of Ab 12 to bind both arms simultaneously.

FIG. 214 shows binding schematics for IgG fusions. Based on FACS and BLI competition data, the BsAb-HC fusion binds in a monovalent manor. As the affinity of Ab 12 is much greater than that of Ab 2-7 and it has been shown that one binding event effectively blocks the second, binding of the BsAb-HC fusion is governed by Ab 12. The BsAb-LC fusion exists in two binding states, both arms of Ab 12 bound and one arm of Ab 2-7 bound. Each of these states exist independent of the other as there is no steric hinderance.

FIG. 215 shows an assay comparison of viral neutralization in different assays and against different viral strains. FIG. 215A) In vitro SARS-CoV-2 neutralization was performed via recombinant nLuc virus and standard PRNT (SARS-CoV-2 isolate WA1 used in both assays). Due to differences in protocol, EC50 values differed, however overall trend was similar. FIG. 215B) In vitro neutralization of D614G virus Antibodies did not show a significant difference in neutralization for D614G and WT virus.

FIG. 216 shows FACS binding curve for tandem scFv-Fcs Tandem scFv-Fcs were bound to SARS-CoV-2 spike expressing cells. Ab 2-7/12 tandem exhibits a decrease in binding efficiency compared to Ab 12/2-7 tandem scFv-Fc.

FIG. 217 shows criteria for lung histopathology scoring HPF—high power field (>10×); PMN—polymorphonuclear cells/heterophils; MNC—mononuclear cells including lymphocytes and macrophages); PVC—Peri-vascular cuff.

FIG. 218 shows graphs of pseudovirus neutralization of D614G, B.1.1.7, and B.1.351. B.1.1.7 (VG40771-UT) and B.1.351 (VG40772-UT) mutant spike cDNA was purchased from Sino Bio and cloned into the pseudovirus spike vector (pcDNA3.4) with a truncated cytoplasmic domain and gp41 tail. LentiX-293T cells were transiently transfected via PEI to generate pseudoviral particles pseudotyped with the various spike proteins. After 3 days culture, the virus containing supernatant was harvested and stored at 4° C. overnight before use in the assay. For the assay, 30 μl ab dilution was mixed with 30 μl virus supernatant and incubated for 60 min at RT, followed by 10 min at 37° C. to warm before adding to cells. The culture media was removed from the 293T-ACE2 cells and replaced with 60 μl of Ab/Virus sup mixture, and cultured for 48 hours at 37° C. After incubation, Ab/virus mixture was removed and replaced with 30 ul DMEM+FBS and allowed to equilibrate to RT on the bench. After equilibration, 30 μl of room temperature bioglo reagent (Promega) as added. Plates were shaken for 15 min at RT, then luciferase was detected on the polarstar omega. B.1.1.7 produced the best pseudovirus with high RLU values (200,000 RLU), D614G was in the middle (virus only=20,000 RLU), but B.1.351 had very low signals (5000 RLU).

FIG. 219 is a graph showing pseudovirus neutralization of SA (B.1.351) virus. B.1.351 shows the most pronounced effect with the Abs in both binding and pseudoneutralization assays. Ab 12 IgG effectively neutralizes D614G and B.1.1.7, however it does not neutralize B.1.351, which correlates to a severe loss of affinity to the variant spike. Ab 2-7 scFv-Fc is not as affected by the mutant spikes and that is also seen in the pseudoneutralization data. Interestingly, the bispecifics appear to retain some benefits of the Ab 2-7 scFv-Fc as neutralization is improved over that of Ab 12 IgG.

FIG. 220 is a bar graph showing scFv-Fc binding to D614G and B.1.1.7 variant spikes via BLI. Anti-SARS-CoV-2 scFv-Fcs were functionalized to AHC sensors prior to Spike capture. Values were normalized to WT binding for each sample.

FIG. 221 is a bar graph showing scFv-Fc binding to B.1.351 and P.1 variant spikes via BLI. Anti-SARS-CoV-2 scFv-Fcs were functionalized to AHC sensors prior to Spike capture. Values were normalized to WT binding for each sample.

FIG. 222 is a bar graph showing bispecific antibody binding to SARS-CoV-2 variant spikes via BLI. Anti-SARS-CoV-2 scFv-Fcs were functionalized to AHC sensors prior to Spike capture. Values were normalized to WT binding for each sample.

FIG. 223 shows graphs of neutralization studies of SARS-CoV2 variants WA 614G, UK (Alpha), Brazil (Gamma), South Africa (Beta), and India-2 (Delta), respectively. Tables depict the numerical values from the graphs as well as the Spike Protein substitutions. Non-limiting examples of amino acid substitutions or deletions of SEQ ID NO: 980 giving rise to the SARS-CoV2 variants include T19R, G142D, Del AA 156-157, R158G, L452R, T478K, D614G, P681R, Del AA 689-691, and D950N.

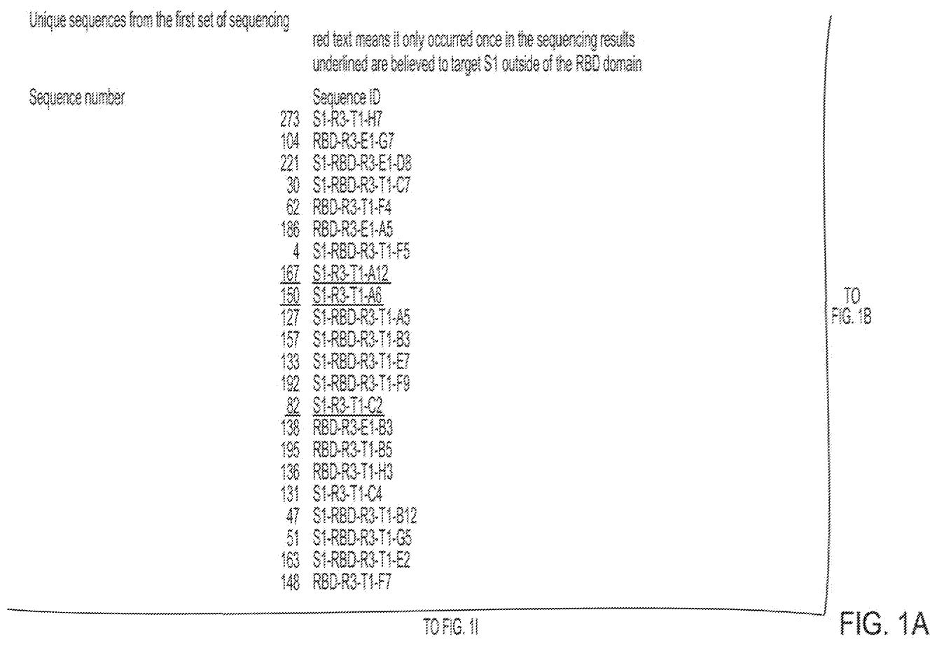

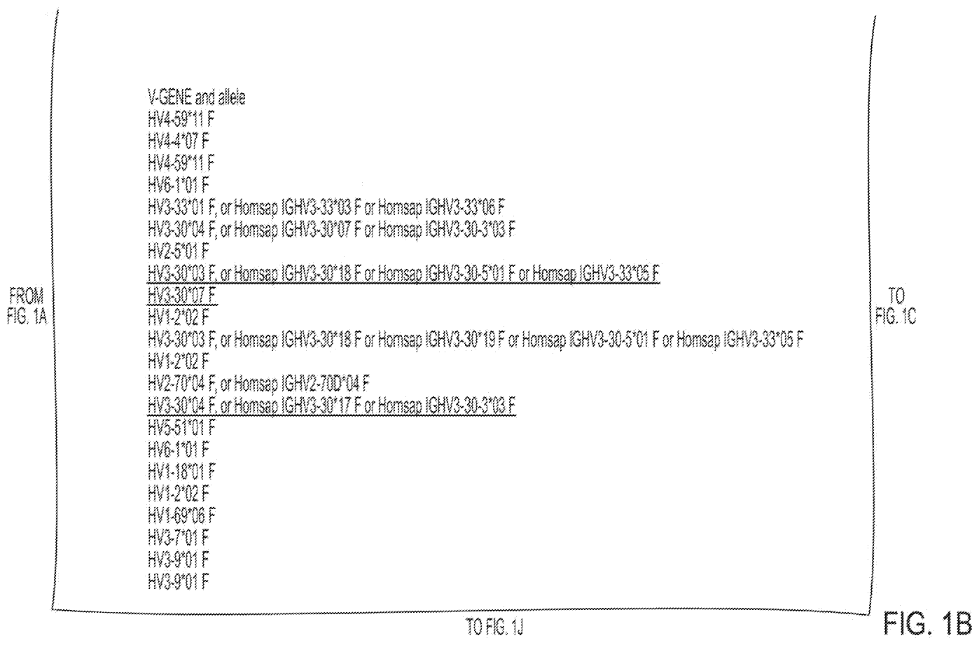

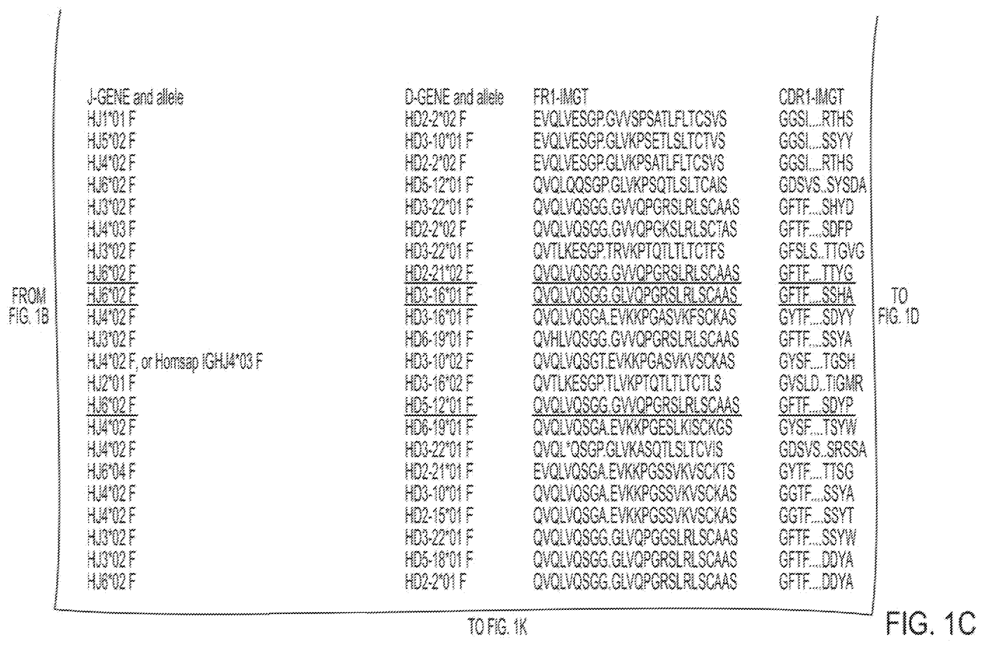

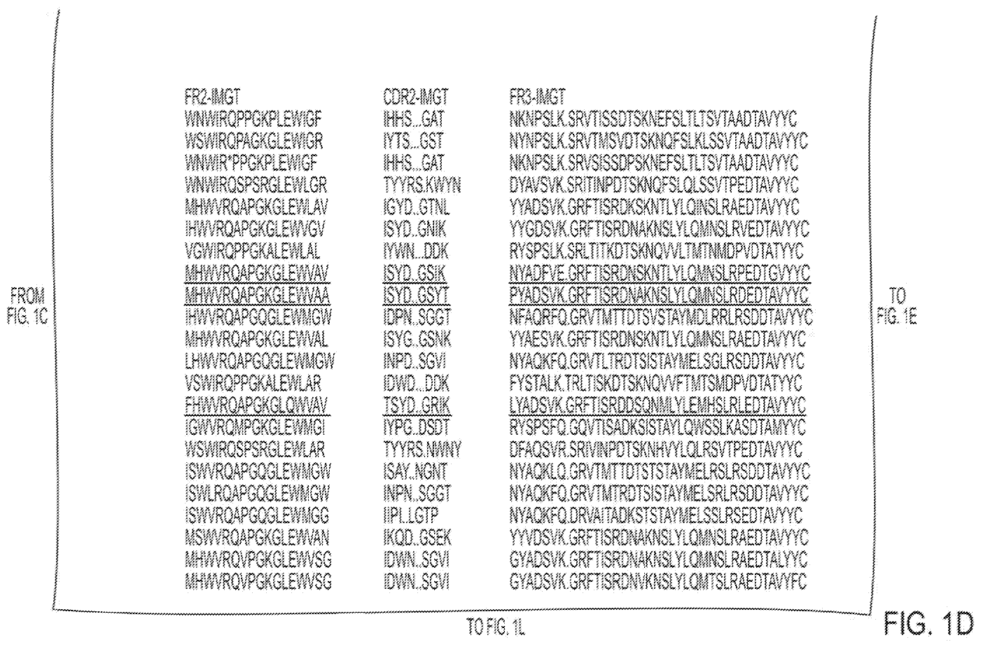

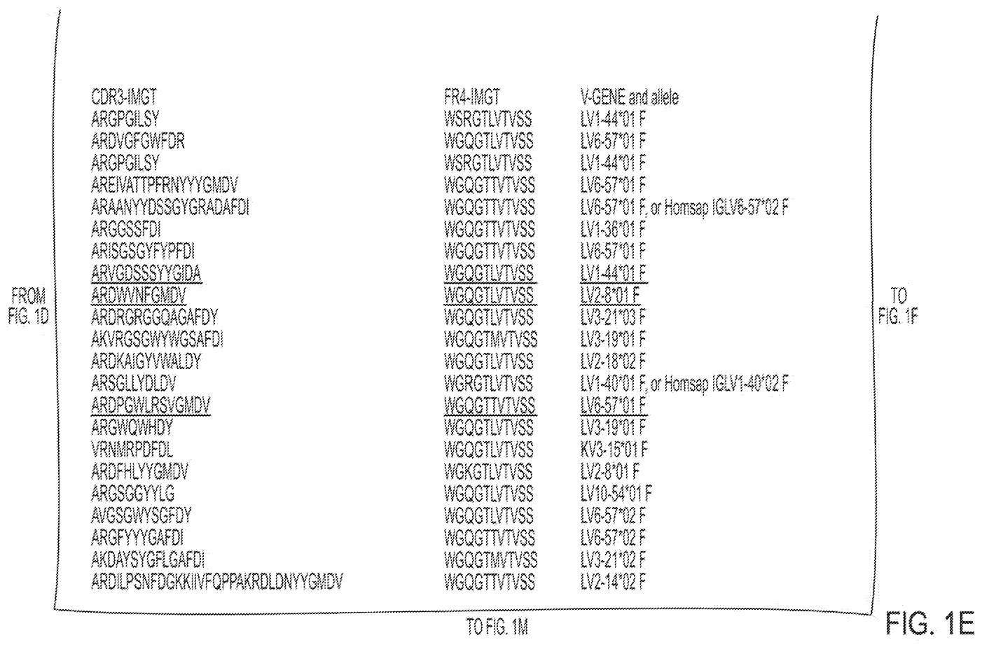

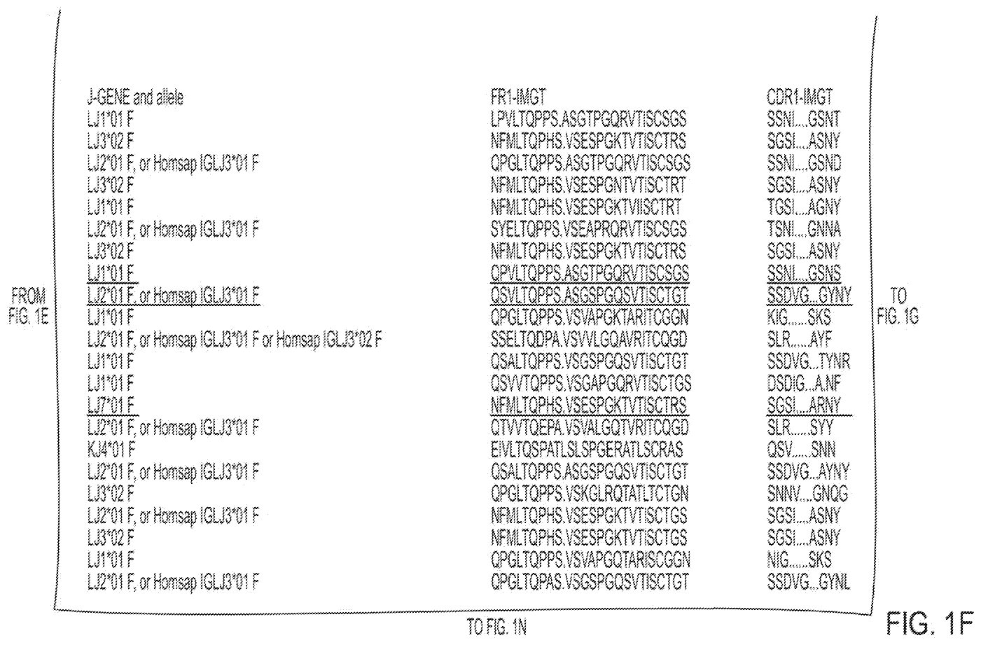

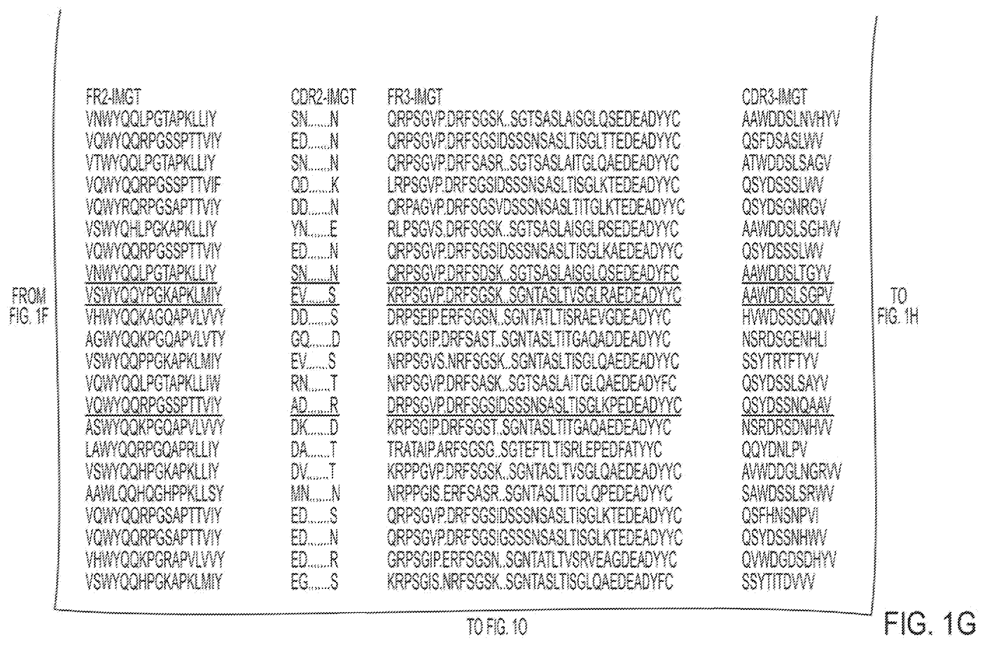

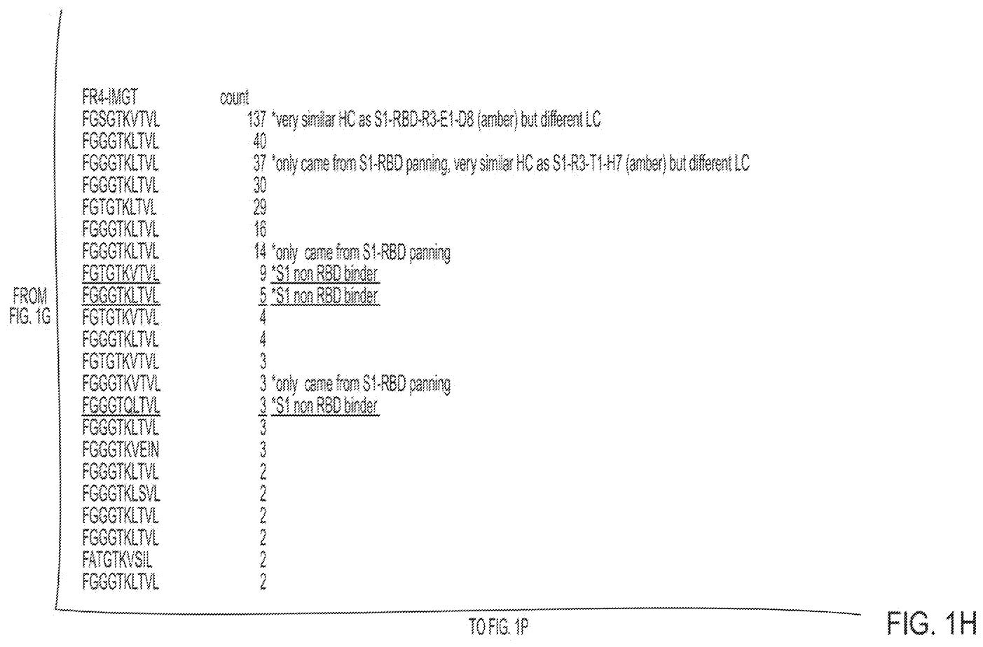

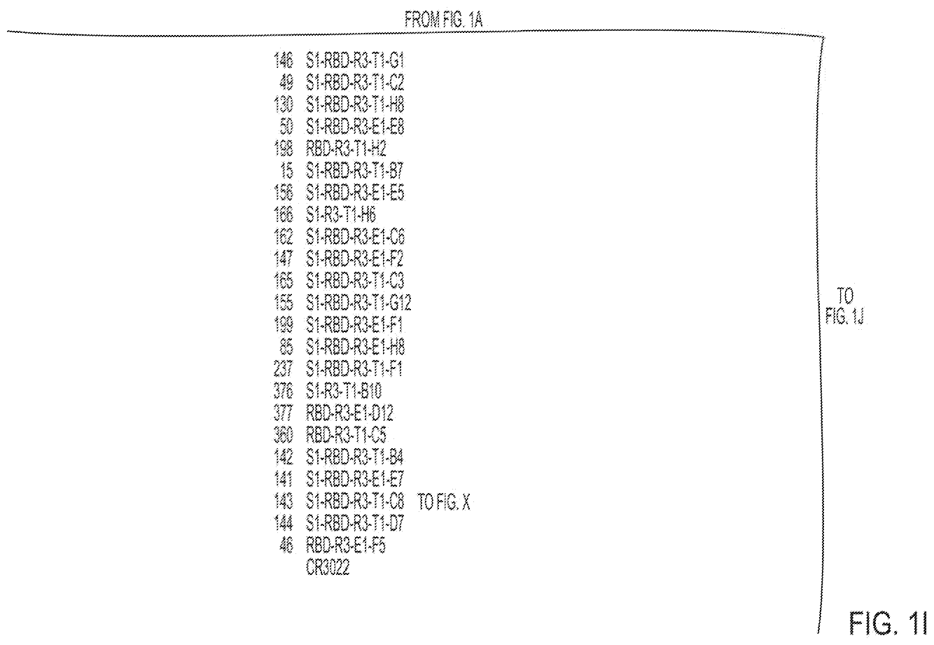

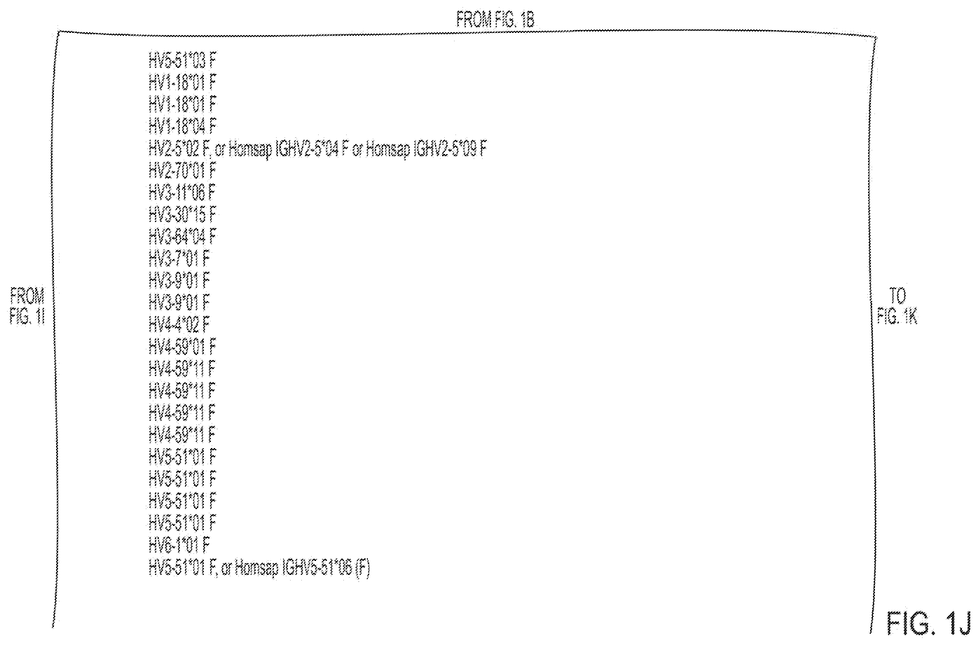

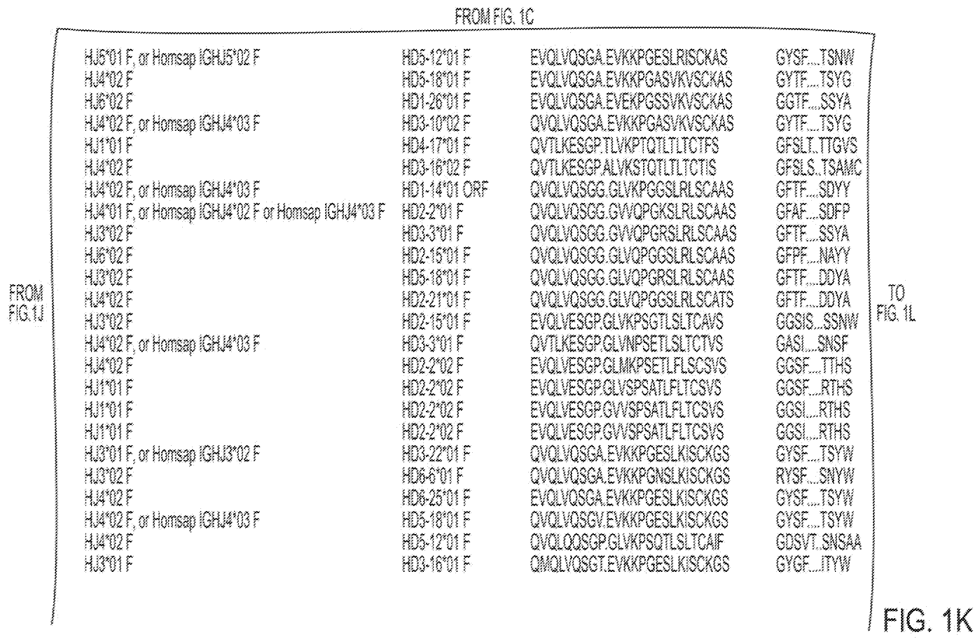

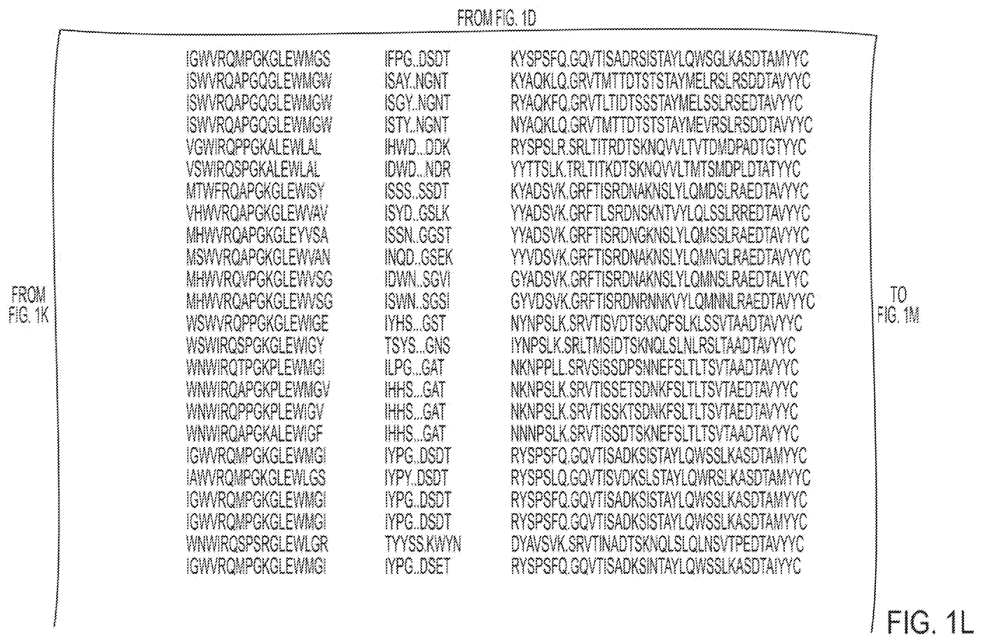

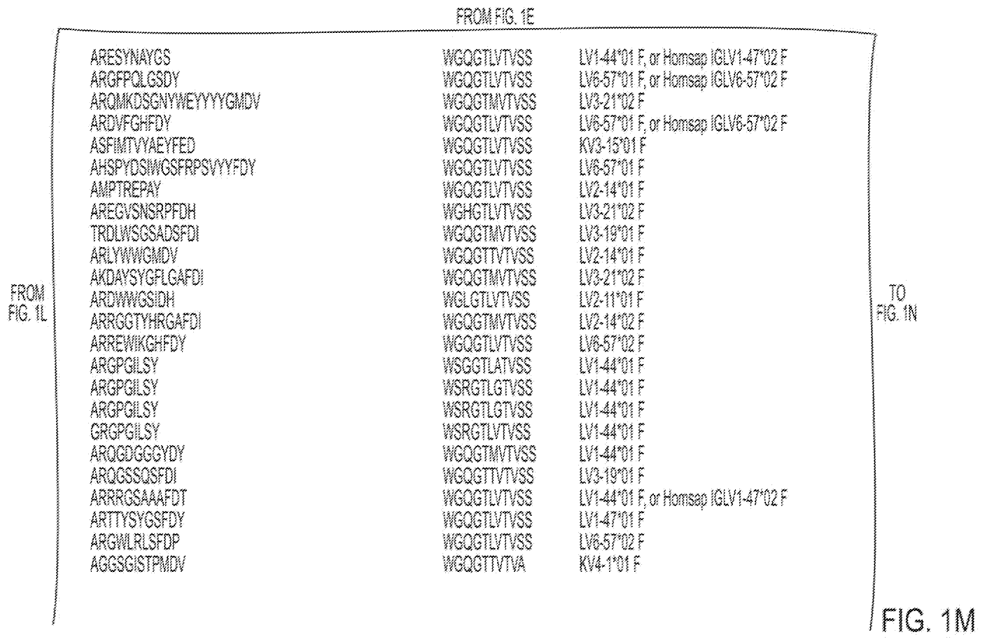

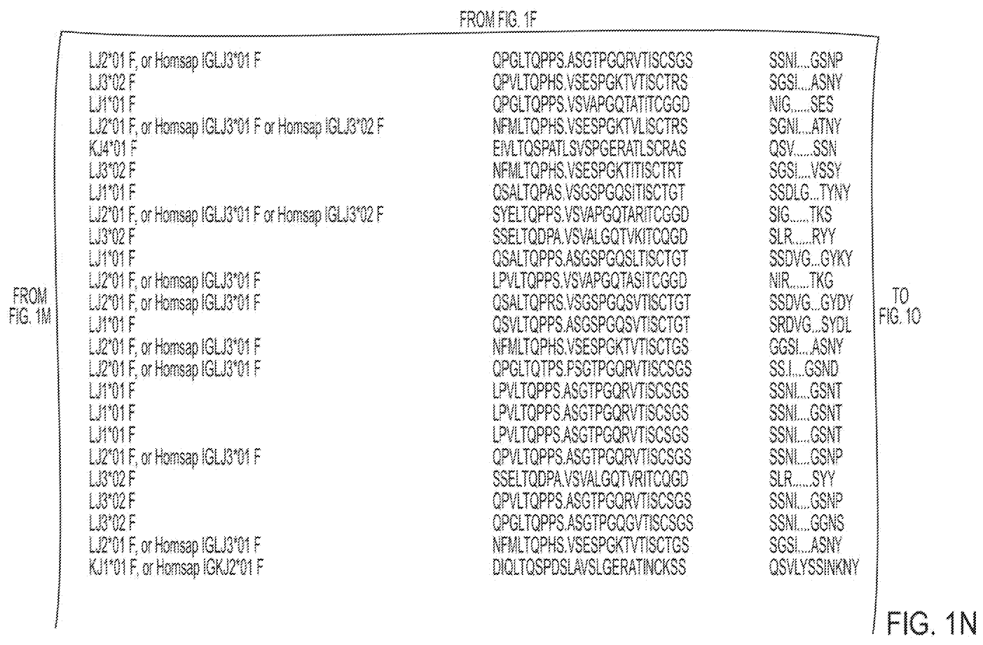

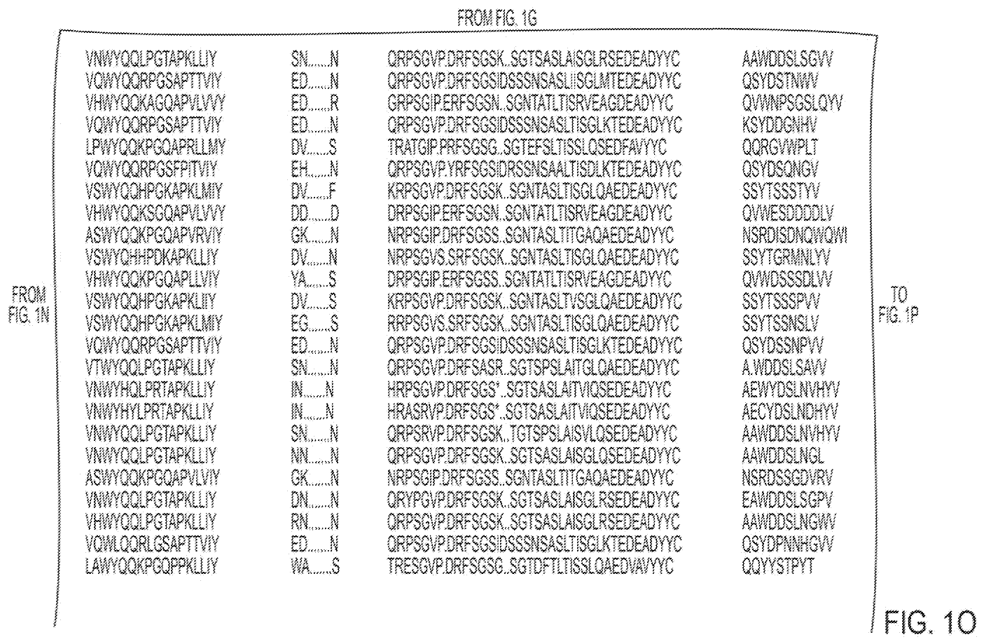

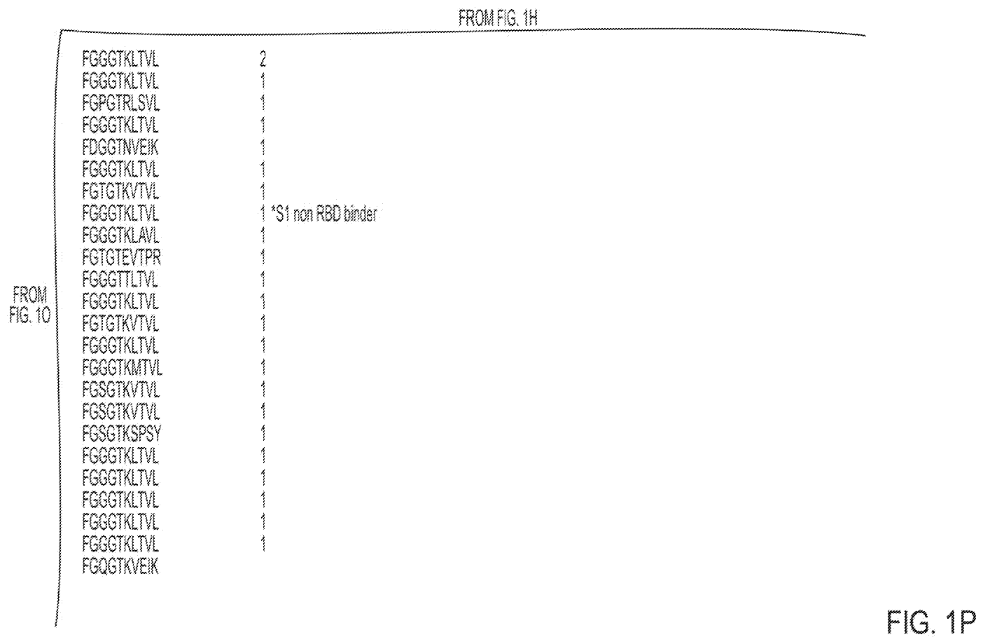

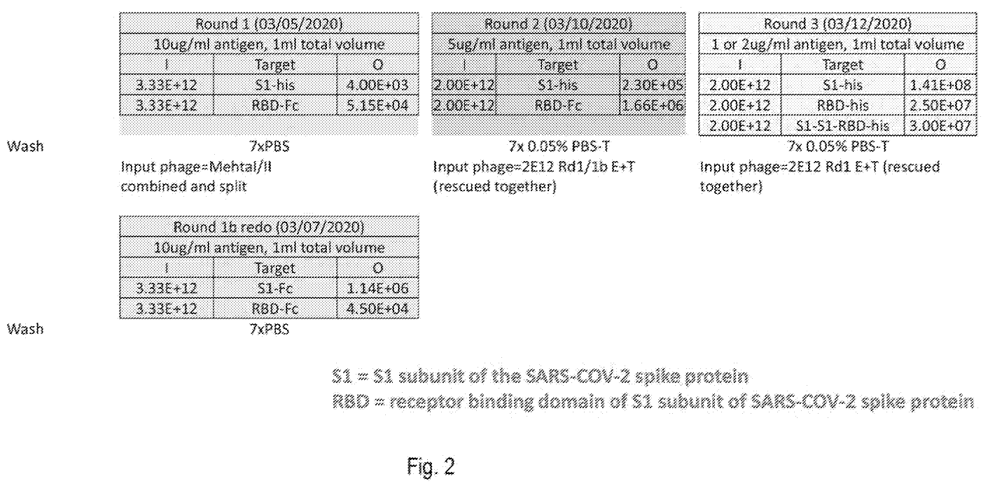

### DETAILED DESCRIPTION

This invention provides antibodies that are directed to severe acute respiratory syndrome-associated coronavirus (SARS-CoV2). In some embodiments, the antibodies described herein can neutralize infection by severe acute respiratory syndrome-associated coronavirus (SARS-CoV2). In other embodiments, the SARS-CoV2 antibodies, for example non-neutralizing antibodies, can be useful for diagnostic purposes. Specifically, anti-SARS-CoV2 Abs were isolated from a non-immune human Ab-phage library using a panning strategy.

The amino acid sequence of the monoclonal SARS-CoV2 antibodies are provided herein; the amino acid sequences of the heavy and light chain complementary determining regions CDRs of the COVID-19 antibodies are underlined (CDR1), underlined and bolded (CDR2), or underlined, italicized, and bolded (CDR3) below:

TABLE 1
S1-R3-T1-H7 (Ab12) Ab Variable Region amino acid sequences

V_{H} chain of H7
EVQLVESGPGVVSPSATLFLTCSVSGGSIRTHSWNWIRQPPGKPLEWIGFIHHSGAT
NKNPSLKSRVTISSDTSKNEFSLTLTSVTAADTAVYYCARGPGILSYWSRGTLVTVS
S
(SEQ ID NO: 1)
V_{L} chain of H7
LPVLTQPPSASGTPGQRVTISCSGSSSNIGSNTVNWYQQLPGTAPKLLIYSNNQRPSG
VP.DRFSGSKSGTSASLAISGLQSEDEADYYCAAWDDSLNVHYVFGSGTKVTVL
(SEQ ID NO: 2)

TABLE 2
RBD-R3-E1-G7 (Ab2-10) Ab Variable Region amino acid sequences

V_{H} chain of G7
EVQLVWSGPGLVPSETLSLTCTVSGGSISSYYWSWIRQPAGKGLEWIGRIYSTGST
NYNPSLKSRVTMSVDTSKNQFSLKLSSVTAADTAVYYCARDVGFGWFDRWGQGT
LVTVSS
(SEQ ID NO: 3)
V_{L} chain of G7
NFMLTQPHSVSESPGKTVTISCTRSSGSIASNYVQWYQQRPGSSPTTVIYEDNQRPS
GVPDRFSGSIDSSSNSASLTISGLTTEDEADYYCQSFDSASLWVFGGGTKLTVL
(SEQ ID NO: 4)

TABLE 3
S1-RBD-R3-E1-D8 (Ab_3) Ab Variable Region amino acid sequences

V_{H} chain of D8
EVQLVESGPGLVKPSATLFLTCSVSGGSIRTHSWNWIR*PPGKPLEWIGFIHHSGAT
NKNPSLKSRVSISSDPSKNEFSLTLTSVTAADTAVYYCARGPGILSYWSRGTLVTVS
S
(SEQ ID NO: 5)
V_{L} chain of D8
QPGLTQPPSASGTPGQRVTISCSGSSSNIGSNDVTWYQQLPGTAPKLLIYSNNQRPSG
VPDRFSASRSGTSASLAITGLQAEDEADYYCATWDDSLSAGVFGGGTKLTVL
(SEQ ID NO: 6)

TABLE 4
S1-RBD-R3-T1-C7 Ab (Ab_29) Variable Region amino acid sequences

V_{H} chain of C7
QVQLQQSGPGLVKPSQTLSLTCAISGDSVSSYSDAWNWIRQSPSRGLEWLGRTYYR
SKWYNDYAVSVKSRITINPDTSKNQFSLQLSSVTPEDTAVYYCAREIVATTPFRNYY
YGMDVWGQGTTVTVSS
(SEQ ID NO: 7)
V_{L} chain of C7
NFMLTQPHSVSESPGNTVTISCTRTSGSIASNYVQWYQQRPGSSPTTVIFQDKLRPS
GVPDRFSGSIDSSSNSASLTISGLKTEDEADYYCQSYDSSSLWVFGGGTKLTVL
(SEQ ID NO: 8)

TABLE 5
RBD-R3-T1-F4 (Ab2-2) Ab Variable Region amino acid sequences

V_{H} chain of F4
QVQLVQSGGGVVQPGRSLRLSCAASGFTFSHYDMHWVRQAPGKGLEWLAVIGYD
GTNLYYADSVKGRFTISRDKSKNTLYLQINSLRAEDTAVYYCARAANYYDSSGYGR
ADAFDIWGQGTTVTVSS
(SEQ ID NO: 9)
V_{L} chain of F4
NFMLTQPHSVSESPGKTVIISCTRTTGSIAGNYVQWYRQRPGSAPTTVIYDDNQRPA
GVPDRFSGSVDSSSNSASLTITGLKTEDEADYYCQSYDSGNRGVFGTGTKLTVL
(SEQ ID NO: 10)

TABLE 6
RBD-R3-E1-A5 Ab (Ab_30) Variable Region amino acid sequences

V_{H} chain of A5
QVQLVQSGGGVVQPGKSLRLSCTASGFTFSDFPIHWVRQAPGKGLEWVGVISYDG
NIKYYGDSVKGRFTISRDNAKNSLYLQMNSLRVEDTAVYYCARGGSSFDIWGQGT
TVTVSS
(SEQ ID NO: 11)
V_{L} chain of A5
SYELTQPPSVSEAPRQRVTISCSGSTSNIGNNAVSWYQHLPGKAPKLLIYYNERLP
SGVSDRFSGSKSGTSASLAISGLRSEDEADYYCAAWDDSLSGHVVFGGGTKLTVL
(SEQ ID NO: 12)

TABLE 7
S1-RBD-R3-T1-F5 (Ab2-7) Ab Variable Region amino acid sequences

V_{H} chain of F5
QVTLKESGPTRVKPTQTLTLTCTFSGFSLSTTGVGVGWIRQPPGKALEWLALIYWN
DDKRYSPSLKSRLTITKDTSKNQVVLTMTNMDPVDTATYYCARISGSGYFYPFDIW
GQGTTVTVSS
(SEQ ID NO: 13)
V_{L} chain of F5
NFMLTQPHSVSESPGKTVTISCTRSSGSIASNYVQWYQQRPGSSPTTVIYEDNQRP
SGVPDRFSGSIDSSSNSASLTISGLKAEDEADYYCQSYDSSSLWVFGGGTKLTVL
(SEQ ID NO: 14)

TABLE 8
S1-R3-T1-A12 (Ab_5) Ab Variable Region amino acid sequences

V_{H} chain of A12
QVQLVQSGGGVVQPGRSLRLSCAASGFTFTTYGMHWVRQAPGKGLEWVAVISYDG
SIKNYADFVEGRFTISRDNSKNTLYLQMNSLRPEDTGVYYCARVGDSSSYYGIDA
WGQGTLVTVSS
(SEQ ID NO: 15)
V_{L} chain of A12
QPVLTQPPSASGTPGQRVTISCSGSSSNIGSNSVNWYQQLPGTAPKLLIYSNNQRP
SGVPDRFSDSKSGTSASLAISGLQSEDEADYFCAAWDDSLTGYVFGTGTKVTVL
(SEQ ID NO: 16)

TABLE 9
S1-R3-T1-A6 (Ab_4) Ab Variable Region amino acid sequences

V_{H} chain of A6
QVQLVQSGGGLVQPGRSLRLSCAASGFTFSSHAMHWVRQAPGKGLEWVAAISYD
GSYTPYADSVKGRFTISRDNAKNSLYLQMNSLRDEDTAVYYCARDWVNFGMDVW
GQGTLVTVSS
(SEQ ID NO: 17)
V_{L} chain of A6
QSVLTQPPSASGSPGQSVTISCTGTSSDVGGYNYVSWYQQYPGKAPKLMIYEVSKR
PSGVPDRFSGSKSGNTASLTVSGLRAEDEADYYCAAWDDSLSGPVFGGGTKLTVL
(SEQ ID NO: 18)

TABLE 10
S1-RBD-R3-T1-A5 Ab (Ab_33) Variable Region amino acid sequences

V_{H} chain of T1-A5
QVQLVQSGAEVKKPGASVKFSCKASGYTFSDYYIHWVRQAPGQGLEWMGWIDPN
SGGTNFAQRFQGRVTMTTDTSVSTAYMDLRRLRSDDTAVYYCARDRGRGGQAGA
FDYWGQGTLVTVSS
(SEQ ID NO: 19)
V_{L} chain of T1-A5
QPGLTQPPSVSVAPGKTARITCGGNKIGSKSVHWYQQKAGQAPVLVVYDDSDRPS
EIPERFSGSNSGNTATLTISRAEVGDEADYYCHVWDSSSDQNVFGTGTKVTVL
(SEQ ID NO: 20)

TABLE 11
S1-RBD-R3-T1-B3 (Ab_1) Ab Variable Region
amino acid sequences

V_{H} chain of B3
QVHLVQSGGGVVQPGRSLRLSCAASGFTFSSYAMHWVRQAPGKGLEWVALISYG
GSNKYYAESVKGRFTISRDNSKNTLYLQMNSLRAEDTAVYYCAKVRGSGWYWGS
AFDIWGQGTMVTVSS
(SEQ ID NO: 21)
V_{L} chain of B3
SSELTQDPAVSVVLGQAVRITCQGDSLRAYFAGWYQQKPGQAPVLVTYGQDKRPS
GIPDRFSASTSGNTASLTITGAQADDEADYYCNSRDSGENHLIFGGGTKLTVL
(SEQ ID NO: 22)

TABLE 12
S1-RBD-R3-T1-E7 Ab Variable Region amino acid sequences

V_{H} chain of E7
QVQLVQSGTEVKKPGASVKVSCKASGYSFTGSHLHWVRQAPGQGLEWMGWINPD
SGVINYAQKFQGRVTLTRDTSISTAYMELSGLRSDDTAVYYCARDKAIGYVWAL
DYWGQGTLVTVSS
(SEQ ID NO: 23)
V_{L} chain of E7
QSALTQPPSVSGSPGQSVTISCTGTSSDVGTYNRVSWYQQPPGKAPKLMIYEVS
NRPSGVSNRFSGSKSGNTASLTISGLQAEDEADYYCSSYTRTFTYVFGTGTKVT
VL
(SEQ ID NO: 24)

TABLE 13
S1-RBD-R3-T1-F9 Ab (Ab_39) Variable Region
amino acid sequences

V_{H} chain of F9
QVTLKESGPTLVKPTQTLTLTCTLSGVSLDTIGMRVSWIRQPPGKALEWLARIDWD
DDKFYSTALKTRLTISKDTSKNQVVFTMTSMDPVDTATYYCARSGLLYDLDVWGR
GTLVTVSS
(SEQ ID NO: 25)
V_{L} chain of F9
QSVVTQPPSVSGAPGQRVTISCTGSDSDIGANFVQWYQQLPGTAPKLLIWRNTNRP
SGVPDRFSASKSGTSASLAITGLQAEDEADYFCQSYDSSLSAYVFGGGTKVTVL
(SEQ ID NO: 26)

TABLE 14
S1-R3-T1-C2 (Ab_6) Ab Variable Region amino acid sequences

V_{H} chain of C2
QVQLVQSGGGVVQPGRSLRLSCAASGFTFSDYPFHWVRQAPGKGLQWVAVTSYD
GRIKLYADSVKGRFTISRDDSQNMLYLEMHSLRLEDTAVYYCARDPGWLRSVGM
DVWGQGTTVTVSS
(SEQ ID NO: 27)
V_{L} chain of C2
NFMLTQPHSVSESPGKTVTISCTRSSGSIARNYVQWYQQRPGSSPTTVIYADRDRPS
GVPDRFSGSIDSSSNSASLTISGLKPEDEADYYCQSYDSSNQAAVFGGGTQLTVL
(SEQ ID NO: 28)

TABLE 15
RBD-R3-E1-B3 Ab (Ab_32) Variable Region
amino acid sequences

V_{H} chain of E1-B3
QVQLVQSGAEVKKPGESLKISCKGSGYSFTSYWIGWVRQMPGKGLEWMGIIYPGD
SDTRYSPSFQGQVTISADKSISTAYLQWSSLKASDTAMYYCARGWQWHDYWGQG
TLVTVSS
(SEQ ID NO: 29)
V_{L} chain of E1-B3
QTVVTQEPAVSVALGQTVRITCQGDSLRSYYASWYQQKPGQAPVLVVYDKDKRPS
GIPDRFSGSTSGNTASLTITGAQAEDEADYYCNSRDRSDNHVVFGGGTKLTVL
(SEQ ID NO: 30)

TABLE 16
RBD-R3-T1-B5 Ab Variable Region amino acid sequences

V_{H} chain of B5
QVQL*QSGPGLVKASQTLSLTCVISGDSVSSRSSAWSWIRQSPSRGLEWLARTYYRS
NWNYDFAQSVRSRIVINPDTSKNHVYLQLRSVTPEDTAVYYCVRNMRPDFDLWG
QGTLVTVSS
(SEQ ID NO: 31)
V_{L} chain of B5
EIVLTQSPATLSLSPGERATLSCRASQSVSNNLAWYQQRPGQAPRLLIYDATTRATA
IPARFSGSGSGTEFTLTISRLEPEDFATYYCQQYDNLPVFGGGTKVEIN
(SEQ ID NO: 32)

TABLE 17
RBD-R3-T1-H3 (Ab_2) Ab Variable Region amino acid sequences

V_{H} chain of H3
EVQLVQSGAEVKKPGSSVKVSCKTSGYTFTTSGISWVRQAPGQGLEWMGWISAYN
GNTNYAQKLQGRVTMTTDTSTSTAYMELRSLRSDDTAVYYCARDFHLYYGMDV
WGKGTLVTVSS
(SEQ ID NO: 33)
V_{L} chain of H3
QSALTQPPSASGSPGQSVTISCTGTSSDVGAYNYVSWYQQHPGKAPKLLIYDVTKR
PPGVPDRFSGSKSGNTASLTVSGLQAEDEADYYCAVWDDGLNGRVVFGGGTKLTV
L
(SEQ ID NO: 34)

TABLE 18
S1-R3-T1-C4 (Ab_8) Ab Variable Region amino acid sequences

V_{H} chain of C4
QVQLVQSGAEVKKPGSSVKVSCKASGGTFSSYAISWLRQAPGQGLEWMGWINPNS
GGTNYAQKFQGRVTMTRDTSISTAYMELSRLRSDDTAVYYCARGSGGYYLGWGQ
GTLVTVSS
(SEQ ID NO: 35)
V_{L} chain of C4
QPGLTQPPSVSKGLRQTATLTCTGNSNNVGNQGAAWLQQHQGHPPKLLSYMNNN
RPPGISERFSASRSGNTASLTITGLQPEDEADYYCSAWDSSLSRWVFGGGTKLSVL
(SEQ ID NO: 36)

TABLE 19
S1-RBD-R3-T1-B12 Ab (Ab_31) Variable Region
amino acid sequences

V_{H} chain of B12
QVQLVQSGAEVKKPGSSVKVSCKASGGTFSSYTISWVRQAPGQGLEWMGGIIPILG
TPNYAQKFQDRVAITADKSTSTAYMELSSLRSEDTAVYYCAVGSGWYSGFDYWGQ
GTLVTVSS
(SEQ ID NO: 37)
V_{L} chain of B12
NFMLTQPHSVSESPGKTVTISCTGSSGSIASNYVQWYQQRPGSAPTTVIYEDSQRPS
GVPDRFSGSIDSSSNSASLTISGLKTEDEADYYCQSFHNSNPVIFGGGTKLTVL
(SEQ ID NO: 38)

TABLE 20
S1-RBD-R3-T1-G5 Ab Variable Region amino acid sequences

V_{H} chain of G5
QVQLVQSGGGLVQPGGSLRLSCAASGFTFSSYWMSWVRQAPGKGLEWVANIKQD
GSEKYYVDSVKGRFTISRDNAKNSLYLQMNSLRAEDTAVYYCARGFYYYGAFDIW
GQGTTVTVSS
(SEQ ID NO: 39)
V_{L} chain of G5
NFMLTQPHSVSESPGKTVTISCTGSSGSIASNYVQWYQQRPGSAPTTVIYEDNQRPS
GVPDRFSGSIGSSSNSASLTISGLKTEDEADYYCQSYDSSNHWVFGGGTKLTVL
(SEQ ID NO: 40)

TABLE 21
S1-RBD-R3-T1-E2 (Ab7) Ab Variable Region amino acid sequences

V_{H} chain of E2
QVQLVQSGGGLVQPGRSLRLSCAASGFTFDDYAMHWVRQVPGKGLEWVSGIDW
NSGVIGY ADSVKGRFTISRDNAKNSLYLQMNSLRAEDTALYYCAKDAYSYGFLGA
FDIWGQGTMVTVSS
(SEQ ID NO: 41)
V_{L} chain of E2
QPGLTQPPSVSVAPGQTARISCGGNNIGSKSVHWYQQKPGRAPVLVVYEDRGRPSG
IPERFSGSNSGNTATLTVSRVEAGDEADYYCQVWDGDSDHYVFATGTKVSIL
(SEQ ID NO: 42)

TABLE 22
RBD-R3-T1-F7 Ab Variable Region amino acid sequences

V_{H} chain of F7
QVQLVQSGGGLVQPGRSLRLSCAASGFTFDDYAMHWVRQVPGKGLEWVSGIDW
NSGVIGYADSVKGRFTISRDNVKNSLYLQMTSLRAEDTAVYFCARDILPSNFD
GKKIIVFQPPAKRDLDNYYGMDVWGQGTTVTVSS
(SEQ ID NO: 43)
V_{L} chain of F7
QPGLTQPASVSGSPGQSVTISCTGTSSDVGGYNLVSWYQQHPGKAPKLMIYEGSK
RPSGISNRFSGSKSGNTASLTISGLQAEDEADYFCSSYTITDVVVFGGGTKLTVL
(SEQ ID NO: 44)

TABLE 23
S1-RBD-R3-T1-G1 Ab (Ab_35) Variable Region
amino acid sequences

V_{H} chain of G1
EVQLVQSGAEVKKPGESLRISCKASGYSFTSNWIGWVRQMPGKGLEWMGSIFPGD
SDTKYSPSFQGQVTISADRSISTAYLQWSGLKASDTAMYYCARESYNAYGSWGQGT
LVTVSS
(SEQ ID NO: 47)
V_{L} chain of G1
QPGLTQPPSASGTPGQRVTISCSGSSSNIGSNPVNWYQQLPGTAPKLLIYSNNQRP
SGVPDRFSGSKSGTSASLAISGLRSEDEADYYCAAWDDSLSGVVFGGGTKLTVL
(SEQ ID NO: 48)

TABLE 24
S1-RBD-R3-T1-C2 Ab Variable Region amino acid sequences

V_{H} chain of RBD-R3-T1-C2
EVQLVQSGAEVKKPGASVKVSCKASGYTFTSYGISWVRQAPGQGLEWMGWISAY
NGNTKYAQKLQGRVTMTTDTSTSTAYMELRSLRSDDTAVYYCARGFPQLGSDYW
GQGTLVTVSS
(SEQ ID NO: 49)
V_{L} chain of RBD-R3-T1-C2
QPVLTQPHSVSESPGKTVTISCTRSSGSIASNYVQWYQQRPGSAPTTVIYEDNQR
PSGVPDRFSGSIDSSSNSASLIISGLMTEDEADYYCQSYDSTNWVFGGGTKLTVL
(SEQ ID NO: 50)

TABLE 25
S1-RBD-R3-T1-H8 Ab Variable Region amino acid sequences

V_{H} chain of RBD-R3-T1-H8
EVQLVQSGAEVEKPGSSVKVSCKASGGTFSSYAISWVRQAPGQGLEWMGWISGYN
GNTRYAQKFQGRVTLTIDTSSSTAYMELSSLRSEDTAVYYCARQMKDSGNYWEYY
YYGMDVWGQGTMVTVSS
(SEQ ID NO: 51)
V_{L} chain of RBD-R3-T1-H8
QPGLTQPPSVSVAPGQTATITCGGDNIGSESVHWYQQKAGQAPVLVVYEDRGRPS
GIPERFSGSNSGNTATLTISRVEAGDEADYYCQVWNPSGSLQYVFGPGTRLSVL
(SEQ ID NO: 52)

TABLE 26
S1-RBD-R3-E1-E8 Ab (Ab_37) Variable Region amino acid sequences
V_{H} chain of RBD-R3-E1-E8
QVQLVQSGAEVKKPGASVKVSCKASGYTFTSYGISWVRQAPGQGLEWMGWISTY
NGNTNYAQKLQGRVTMTTDTSTSTAYMEVRSLRSDDTAVYYCARDVFGHFDYW
GQGTLVTVSS
(SEQ ID NO: 53)
V_{L} chain of RBD-R3-E1-E8
NFMLTQPHSVSESPGKTVLISCTRSSGNIATNYVQWYQQRPGSAPTTVIYEDNQRPS
GVPDRFSGSIDSSSNSASLTISGLKTEDEADYYCKSYDDGNHVFGGGTKLTVL
(SEQ ID NO: 54)

TABLE 27
RBD-R3-T1-H2 Ab Variable Region amino acid sequences
V_{H} chain of RBD-R3-T1-H2
QVTLKESGPTLVKPTQTLTLTCTFSGFSLTTTGVSVGWIRQPPGKALEWLALIHWD
DDKRYSPSLRSRLTITRDTSKNQVVLTVTDMDPADTGTYYCASFIMTVYAEYFED
WGQGTLVTVSS
(SEQ ID NO: 55)
V_{L} chain of RBD-R3-T1-H2
EIVLTQSPATLSVSPGERATLSCRASQSVSSNLPWYQQKPGQAPRLLMYDVSTRAT
GIPPRFSGSGSGTEFSLTISSLQSEDFAVYYCQQRGVWPLTFDGGTNVEIK
(SEQ ID NO: 56)

TABLE 28
S1-RBD-R3-T1-B7 Ab Variable Region amino acid sequences
V_{H} chain of RBD-R3-T1-B7
QVTLKESGPALVKSTQTLTLTCTISGFSLSTSAMCVSWIRQSPGKALEWLALIDWD
NDRYYTTSLKTRLTITKDTSKNQVVLTMTSMDPLDTATYYCAHSPYDSIWGSFRPS
VYYFDYWGQGTLVTVSS
(SEQ ID NO: 57)
V_{L} chain of RBD-R3-T1-B7
NFMLTQPHSVSESPGKTITISCTRTSGSIVSSYVQWYQQRPGSFPITVIYEHNQRPSG
VPYRFSGSIDRSSNSAALTISDLKTEDEADYYCQSYDSQNGVFGGGTKLTVL
(SEQ ID NO: 58)

TABLE 29
S1-RBD-R3-E1-E5 Ab Variable Region amino acid sequences
V_{H} chain of RBD-R3-E1-E5
QVQLVQSGGGLVKPGGSLRLSCAASGFTFSDYYMTWFRQAPGKGLEWISYISSSSS
DTKYADSVKGRFTISRDNAKNSLYLQMDSLRAEDTAVYYCAMPTREPAYWGQGT
LVTVSS
(SEQ ID NO: 59)
V_{L} chain of RBD-R3-E1-E5
QSALTQPASVSGSPGQSITISCTGTSSDLGTYNYVSWYQQHPGKAPKLMIYDVFKRP
SGVPDRFSGSKSGNTASLTISGLQAEDEADYYCSSYTSSSTYVFGTGTKVTVL
(SEQ ID NO: 60)

TABLE 30
S1-R3-T1-H6 Ab (Ab_40) Variable Region amino acid sequences
V_{H} chain of RBD-R3-T1-H6
QVQLVQSGGGVVQPGKSLRLSCAASGFAFSDFPVHWVRQAPGKGLEWVAVISYD
GSLKYYADSVKGRFTLSRDNSKNTVYLQLSSLRREDTAVYYCAREGVSNSRPFDH
WGHGTLVTVSS
(SEQ ID NO: 61)
V_{L} chain of RBD-R3-T1-H6
SYELTQPPSVSVAPGQTARITCGGDSIGTKSVHWYQQKSGQAPVLVVYDDDDRPSG
IPERFSGSNSGNTATLTISRVEAGDEADYYCQVWESDDDDLVFGGGTKLTVL
(SEQ ID NO: 62)

TABLE 31
S1-RBD-R3-E1-C6 Ab Variable Region amino acid sequences
V_{H} chain of RBD-R3-E1-C6
QVQLVQSGGGVVQPGRSLRLSCAASGFTFSSYAMHWVRQAPGKGLEYVSAISSNG
GSTYYADSVKGRFTISRDNGKNSLYLQMSSLRAEDTAVYYCTRDLWSGSADSFDI
WGQGTMVTVSS
(SEQ ID NO: 63)
V_{L} chain of RBD-R3-E1-C6
SSELTQDPAVSVALGQTVKITCQGDSLRRYYASWYQQKPGQAPVRVIYGKNNRPS
GIPDRFSGSSSGNTASLTITGAQAEDEADYYCNSRDISDNQWQWIFGGGTKLAVL
(SEQ ID NO: 64)

TABLE 32
S1-RBD-R3-E1-F2 Ab Variable Region amino acid sequences
V_{H} chain of RBD-R3-E1-F2
QVQLVQSGGGLVQPGGSLRLSCAASGFPFNAYYMSWVRQAPGKGLEWVANINQD
GSEKYYVDSVKGRFTISRDNAKNSLYLQMNGLRAEDTAVYYCARLYWWGMDVW
GQGTTVTVSS
(SEQ ID NO: 65)
V_{L} chain of RBD-R3-E1-F2
QSALTQPPSASGSPGQSLTISCTGTSSDVGGYKYVSWYQHHPDKAPKLLIYDVNNR
PSGVSSRFSGSKSGNTASLTISGLQAEDEADYYCSSYTGRMNLYVFGTGTEVTPR
(SEQ ID NO: 66)

TABLE 33
S1-RBD-R3-T1-C3 Ab Variable Region amino acid sequences
V_{H} chain of RBD-R3-T1-C3
QVQLVQSGGGLVQPGRSLRLSCAASGFTFDDYAMHWVRQVPGKGLEWVSGIDW
NSGVIGYADSVKGRFTISRDNAKNSLYLQMNSLRAEDTALYYCAKDAYSYGFLGA
FDIWGQGTMVTVSS
(SEQ ID NO: 67)
V_{L} chain of RBD-R3-T1-C3
LPVLTQPPSVSVAPGQTASITCGGDNIRTKGVHWYQQKPGQAPLLVIYYASDRPSGI
PERFSGSSSGNTATLTISRVEAGDEADYYCQVWDSSSDLVVFGGGTTLTVL
(SEQ ID NO: 68)

TABLE 34
S1-RBD-R3-T1-G12 Ab Variable Region amino acid sequences
V_{H} chain of RBD-R3-T1-G12
QVQLVQSGGGLVQPGGSLRLSCATSGFTFDDYAMHWVRQAPGKGLEWVSGISWN
SGSIGYVDSVKGRFTISRDNRNNKVYLQMNNLRAEDTAVYYCARDWWGSIDHWG
LGTLVTVSS
(SEQ ID NO: 69)
V_{L} chain of RBD-R3-T1-G12
QSALTQPRSVSGSPGQSVTISCTGTSSDVGGYDYVSWYQQHPGKAPKLIIYDVSKRP
SGVPDRFSGSKSGNTASLTVSGLQAEDEADYYCSSYTSSSPVVFGGGTKLTVL
(SEQ ID NO: 70)

TABLE 35
S1-RBD-R3-E1-F1 Ab Variable Region amino acid sequences
V_{H} chain of RBD-R3-E1-F1
EVQLVESGPGLVKPSGTLSLTCAVSGGSISSSNWWSWVRQPPGKGLEWIGEIYHSG
STNYNPSLKSRVTISVDTSKNQFSLKLSSVTAADTAVYYCARRGGTYHRGAFDIWG
QGTMVTVSS
(SEQ ID NO: 71)
V_{L} chain of RBD-R3-E1-F1
QSVLTQPPSASGSPGQSVTISCTGTSRDVGSYDLVSWYQQHPGKAPKLMIYEGSRR
PSGVSSRFSGSKSGNTASLTISGLQAEDEADYYCSSYTSSNSLVFGTGTKVTVL
(SEQ ID NO: 72)

TABLE 36
S1-RBD-R3-E1-H8 Ab Variable Region amino acid sequences
V_{H} chain of RBD-R3-E1-H8
QVTLKESGPGLVNPSETLSLTCTVSGASISNSFWSWIRQSPGKGLEWIGYTSYSGNSI
YNPSLKSRLTMSIDTSKNQLSLNLRSLTAADTAVYYCARREWIKGHFDYWGQGTL
VTVSS
(SEQ ID NO: 73)
V_{L} chain of RBD-R3-E1-H8
NFMLTQPHSVSESPGKTVTISCTGSGGSIASNYVQWYQQRPGSAPTTVIYEDNQRPS
GVPDRFSGSIDSSSNSASLTISGLKTEDEADYYCQSYDSSNPVVFGGGTKLTVL
(SEQ ID NO: 74)

TABLE 37
S1-RBD-R3-T1-F1 Ab Variable Region amino acid sequences
V_{H} chain of RBD-R3-T1-F1
EVQLVESGPGLMKPSETLFLSCSVSGGSFTTHSWNWIRQTPGKPLEWMGIILPGGA
TNKNPPLLSRVSISSDPSNNEFSLTLTSVTAADTAVYYCARGPGILSYWSGGTLATV
SS
(SEQ ID NO: 75)
V_{L} chain of RBD-R3-T1-F1
QPGLTQTPSPSGTPGQRVTISCSGSSSIGSNDVTWYQQLPGTAPKLLIYSNNQRPSGV
PDRFSASRSGTSPSLAITGLQAEDEADYYCAWDDSLSAVVFGGGTKMTVL
(SEQ ID NO: 76)

TABLE 38
S1-R3-T1-B10 Ab Variable Region amino acid sequences
V_{H} chain of R3-T1-B10
EVQLVESGPGLVSPSATLFLTCSVSGGSFRTHSWNWIRQAPGKPLEWMGVIHHSGA
TNKNPSLKSRVTISSETSDNKFSLTLTSVTAEDTAVYYCARGPGILSYWSRGTLGTV
SS
(SEQ ID NO: 77)
V_{L} chain of R3-T1-B10
LPVLTQPPSASGTPGQRVTISCSGSSSNIGSNTVNWYHQLPRTAPKLLIYINNHRPSG
VPDRFSGS*SGTSASLAITVIQSEDEADYYCAEWYDSLNVHYVFGSGTKVTVL
(SEQ ID NO: 78)

TABLE 39
RBD-R3-E1-D12 Ab Variable Region amino acid sequences
V_{H} chain of RBD-R3-E1-D12
EVQLVESGPGVVSPSATLFLTCSVSGGSIRTHSWNWIRQPPGKPLEWIGVIHHSGAT
NKNPSLKSRVTISSKTSDNKFSLTLTSVTAEDTAVYYCARGPGILSYWSRGTLGTVS
S
(SEQ ID NO: 79)
V_{L} chain of RBD-R3-E1-D12
LPVLTQPPSASGTPGQRVTISCSGSSSNIGSNTVNWYHYLPRTAPKLLIYINNHRASR
VPDRFSGS*SGTSASLAITVIQSEDEADYYCAECYDSLNDHYVFGSGTKVTVL
(SEQ ID NO: 80)

TABLE 40
RBD-R3-T1-C5 Ab Variable Region amino acid sequences
V_{H} chain of RBD-R3-T1-C5
EVQLVESGPGVVSPSATLFLTCSVSGGSIRTHSWNWIRQAPGKALEWIGFIHHSGAT
NNNPSLKSRVTISSDTSKNEFSLTLTSVTAADTAVYYCGRGPGILSYWSRGTLVTVS
S
(SEQ ID NO: 81)
V_{L} chain of RBD-R3-T1-C5
LPVLTQPPSASGTPGQRVTISCSGSSSNIGSNTVNWYQQLPGTAPKLLIYSNNQRPSR
VPDRFSGSKTGTSPSLAISVLQSEDEADYYCAAWDDSLNVHYVFGSGTKSPSY
(SEQ ID NO: 82)

TABLE 41
S1-RBD-R3-T1-B4 Ab Variable Region amino acid sequences
V_{H} chain of RBD-R3-T1-B4
QVQLVQSGAEVKKPGESLKISCKGSGYSFTSYWIGWVRQMPGKGLEWMGIIYPGD
SDTRYSPSFQGQVTISADKSISTAYLQWSSLKASDTAMYYCARQGDGGGYDYWGQ
GTMVTVSS
(SEQ ID NO: 83)
V_{L} chain of RBD-R3-T1-B4
QPVLTQPPSASGTPGQRVTISCSGSSSNIGSNPVNWYQQLPGTAPKLLIYNNNQRPS
GVPDRFSGSKSGTSASLAISGLQSEDEADYYCAAWDDSLNGLFGGGTKLTVL
(SEQ ID NO: 84)

TABLE 42
S1-RBD-R3-E1-E7 Ab Variable Region amino acid sequences
V_{H} chain of RBD-R3-E1-E7
QVQLVQSGAEVKKPGNSLKISCKGSRYSFSNYWIAWVRQMPGKGLEWLGSIYPYD
SDTRYSPSLQGQVTISVDKSLSTAYLQWRSLKASDTAMYYCARQGSSQSFDIWGQ
GTTVTVSS
(SEQ ID NO: 85)
V_{L} chain of RBD-R3-E1-E7
SSELTQDPAVSVALGQTVRITCQGDSLRSYYASWYQQKPGQAPVLVIYGKNNRPS
GIPDRFSGSSSGNTASLTITGAQAEDEADYYCNSRDSSGDVRVFGGGTKLTVL
(SEQ ID NO: 86)

TABLE 43
S1-RBD-R3-T1-C8 Ab Variable Region amino acid sequences
V_{H} chain of RBD-R3-T1-C8
EVQLVQSGAEVKKPGESLKISCKGSGYSFTSYWIGWVRQMPGKGLEWMGIIYPGD
SDTRYSPSFQGQVTISADKSISTAYLQWSSLKASDTAMYYCARRRGSAAAFDTWGQ
GTLVTVSS
(SEQ ID NO: 87)
V_{L} chain of RBD-R3-T1-C8
QPVLTQPPSASGTPGQRVTISCSGSSSNIGSNPVNWYQQLPGTAPKLLIYDNNQRYP
GVPDRFSGSKSGTSASLAISGLRSEDEADYYCEAWDDSLSGPVFGGGTKLTVL
(SEQ ID NO: 88)

TABLE 44
S1-RBD-R3-T1-D7 Ab (Ab_34) Variable Region amino acid sequences
V_{H} chain of RBD-R3-T1-D7
QVQLVQSGVEVKKPGESLKISCKGSGYSFTSYWIGWVRQMPGKGLEWMGIIYPGD
SDTRYSPSFQGQVTISADKSISTAYLQWSSLKASDTAMYYCARTTYSYGSFDYWGQ
GTLVTVSS
(SEQ ID NO: 89)
V_{L} chain of RBD-R3-T1-D7
QPGLTQPPSASGTPGQGVTISCSGSSSNIGGNSVHWYQQLPGTAPKLLIYRNNQRPS
GVPDRFSGSKSGTSASLAISGLRSEDEADYYCAAWDDSLNGWVFGGGTKLTVL
(SEQ ID NO: 90)

TABLE 45
RBD-R3-E1-F5 Ab (Ab_36) Variable Region amino acid sequences
V_{H} chain of RBD-R3-E1-F5
QVQLQQSGPGLVKPSQTLSLTCAIFGDSVTSNSAAWNWIRQSPSRGLEWLGRTYYS
SKWYNDYAVSVKSRVTINADTSKNQLSLQLNSVTPEDTAVYYCARGWLRLSFDP
WGQGTLVTVSS
(SEQ ID NO: 91)
V_{L} chain of RBD-R3-E1-F5
NFMLTQPHSVSESPGKTVTISCTGSSGSIASNYVQWLQQRLGSAPTTVIYEDNQRPS
GVPDRFSGSIDSSSNSASLTISGLKTEDEADYYCQSYDPNNHGVVFGGGTKLTVL
(SEQ ID NO: 92)

TABLE 46
Ab_13 Variable Region amino acid sequences
V_{H} chain of Ab_13
QVTLKESGPKLVKPTQTLTLTCTFSGFSLSTSGVGVGWIRQPPGKALEWLALIYWD
DDKRYSPSLKSRLTIAKDTSKYQVVLTMTNMDPVDTATYYCARISGSGYFYPFDIW
GQGTMVTVSA
(SEQ ID NO: 722)
V_{L} chain of Ab_13
NFMLTQPHSVSESPGKTVTISCTRSSGSIASNYVQWYQQRPGSAPTTVIYEDNQRPS
GVPDRFSGSIDSSSNSASLTISGLKTEDEADYYCQSYDSSNLWVFGGGTKLTVL
(SEQ ID NO: 723)

TABLE 47
Ab_14 Variable Region amino acid sequences
V_{H} chain of Ab_14
QVQLQQSGPGLVKPSQTLSLTCAISGDSVSSNSAAWNWIRQSPARGLEWLGRTYY
RSRWYNDYAISMKSRITINPDTSKNQFSLQLESVTPEDTAVYYCAREIRGFDYWGQ
GTLVTVSS
(SEQ ID NO: 724)
V_{L} chain of Ab_14
QSVLTQPPSASGSPGQSVTISCTGTSSDVGAYNFVSWYQHHPGKAPKLIIYDFNKRP
SGVPDRFSGSKSGNTASLTVSGLQADDEADYYCSSYAGSNNFDVVFGGGTKLTVL
(SEQ ID NO: 725)

TABLE 48
Ab_15 Variable Region amino acid sequences
V_{H} chain of Ab_15
QVQLVQSGGGLVKPGRSLRLSCTASGFTFGDYAMSWFRQAPGKGLEWVGFIRSKA
YGGTTGYAASVKGRFTISRDDSKSIAYLQMNSLKTEDTAVYYCTTADDDMDVWG
KGTTVTVSS
(SEQ ID NO: 726)
V_{L} chain of Ab_15
NFMLTQPHSVSESPGKTVTISCTRSSGTIASNYVQWYQQRPGSAPTTVIYEDNQRPS
GVPDRFSGSIDRSSNSASLTISGLTPDDEADYYCQSYDTSNHYVFGTGTKVTVL
(SEQ ID NO: 727)

TABLE 49
Ab_16 Variable Region amino acid sequences
V_{H} chain of Ab_16
QVQLVQSGGGVVQPGRSLRLSCAASGFTFSNYGMHWVRQAPGKGLEWVALIWER
GSKKDYADSVKGRFTVSRDNSKNTLYLQMNSLRPEDTAVYFCAREGISMTGAEYF
QHWGQGTLVTVSS
(SEQ ID NO: 728)
V_{L} chain of Ab_16
QSVLTQPPSVSGSPGQRVTMSCTGSSSNIGAGYDVHWYQQVPGAAPRLLIYGTNNR
PSGVPDRFSGSKSGTSASLTITGLQAEDEADYYCQSYDNSLTDPYVFGTGTKVTVL
(SEQ ID NO: 729)

TABLE 50
Ab_17 Variable Region amino acid sequences
V_{H} chain of Ab_17
QVQLVQSGGGVVQPGRSLRLSCAASGFTFDDYAMHWVRQVPGKGLEWVSGIDW
NSGVIGYADSVKGRFTISRDNVKNSLYLQMNSLRTEDSALYYCAKDIGPGGSGSYY
AFDIWGQGTTVTVSS
(SEQ ID NO: 730)
V_{L} chain of Ab_17
QSALTQPASVSGSPGQSITISCTGTSSDVGGSKYVSWYQQHPGKAPKVMIYDVTKR
PSGVPDRFSGSKSGNTASLTISGLQAEDEADYYCAAWDDSLNGVVFGGGTKLTVL
(SEQ ID NO: 731)

TABLE 51
Ab_18 Variable Region amino acid sequences
V_{H} chain of Ab_18
EVQLVQSGGGVVQPGRSLRVSCAASGFSFSRYGMHWVRQAPGKGLEWVAFIRHD
GSKKYYADSVEGRFTISRDNSRNTVSLEMNSLRGEDTAVYYCAKDGRLEAALDDW
GQGTLVTVSS
(SEQ ID NO: 732)
V_{L} chain of Ab_18
NFMLTQPHSMSGSAGKTVTVSCIRSSGSIANNFVQWYQQRPGSAPTTVIYEDNQRP
SGVPDRFSGSIDSSSNSASLTISGLKTEDEADYYCQSYDSSNLVFGGGTRLTVL
(SEQ ID NO: 733)

TABLE 52
Ab_19 Variable Region amino acid sequences
VH chain of Ab_19
EVQLVESGAEVKKPGESLKISCKGSGYSFTSYWIGWVRQMPGKGLEWMGIIYPGD
SDTRYSPSFQGQVTISADKSISTAYLQWSSLKASDTAMYYCARRGDLDAFDIWGQG
TMVTVSS
(SEQ ID NO: 734)
VL chain of Ab_19
QPGLTQPPAASGTPGQRVTVSCSGASANIGSNAVSWFKQFPETAPRLLISGNTHRPS
GVPDRVSGSKSGTSASLTISGLQSDDEADYYCAAWDDSLNGYVFGSGTKVTVL
(SEQ ID NO: 735)

TABLE 53
Ab_20 Variable Region amino acid sequences
VH chain of Ab_20
QVQLVQSGGGSVKPGGSLRLSCAASGYRLSDYYMHWVRQAPGKGLEWVANIKQ
DGSEKYYVDSVKGRFTISRDNAKNSLYLQMNSLRAEDTAVYYCARVRGWSRGYF
DYWGQGTLVTVSS
(SEQ ID NO: 736)
VL chain of Ab_20
NFMLTQPHSVSESPGKTVTISCTGSSGSIASNYVQWYQQRPGSAPTTVIYEDNQRPS
GVPDRFSGSIDSSSNSASLLISGLKTEDEADYYCQSYDSSNHWVFGGGTKLTVL
(SEQ ID NO: 737)

TABLE 54
Ab_21 Variable Region amino acid sequences
VH chain of Ab_21
QVQLVQSGGGLVQPGGSLRLSCATSGFTFDDYAMHWVRQAPGKGLEWVSGISWN
SGSIGYVDSVKGRFTISRDNRNNKVYLQMNNLRAEDTAVYYCARDWWGSIDHWG
LGTLVTVSS
(SEQ ID NO: 738)
VL chain of Ab_21
QSALTQPRSVSGSPGQSVTISCTGTSSDVGGYDYVSWYQQHPGKAPKLIIYDVSKRP
SGVPDRFSGSKSGNTASLTVSGLQAEDEADYYCSSYTSSSPVVFGGGTKLTVL
(SEQ ID NO: 739)

TABLE 55
Ab_22 Variable Region amino acid sequences
VH chain of Ab_22
QVQLVQSGGGVVQPGRSLRLSCAASGFTFSHYDMHWVRQAPGKGLEWLAVIGYD
GTNLYYADSVKGRFTISRDKSKNTLYLQINSLRAEDTAVYYCARAANYYDSSGYGR
ADAFDIWGQGTTVTVSS
(SEQ ID NO: 740)
VL chain of Ab_22
NFMLTQPHSVSESPGKTVIISCTRTTGSIAGNYVQWYRQRPGSAPTTVIYDDNQRPA
GVPDRFSGSVDSSSNSASLTITGLKTEDEADYYCQSYDSGNRGVFGTGTKLTVL
(SEQ ID NO: 741)

TABLE 56
Ab_23 Variable Region amino acid sequences
V_{H} chain of Ab_23
EVQLVQSGAEVKKPGSSVKVSCRSSGGTFSTYGITWVRQAPGQGLEWMGRIIPSLG
IPNYAQKFQGRVTITADTSVSTAWMELSSLESDDTAIYYCARENIDLATNDFWGQG
TLVTVSS
(SEQ ID NO: 742)
V_{L} chain of Ab_23
QSALTQPPSASGSPGQSVTISCTGTSRDIGAYGYVSWYQQVPGKAPKLIIYEVRNRP
SGVSSRFSGSKSGNTASLTISGLQAEDEADYYCSSYTSSSTLDVVFGGGTKLTVL
(SEQ ID NO: 743)

TABLE 57
Ab_24 Variable Region amino acid sequences
V_{H} chain of Ab_24
QVQLVQSGAEVKTPGSSVKVSCKASGGTFSSSGVSWVRQAPGQGLEWMGGIIPML
GTPNYAQKFQGRITITADEATSTVYMALSSLRSEDTAMYYCARDGGNYDYWGQGT
LVTVSS
(SEQ ID NO: 744)
V_{L} chain of Ab_24
QPVLTQPPSASGTPGQRVSISCSGSSSNIGRNAVDWYHQVPGTAPQLLIYSNNERSS
GVPDRFSASRSGNTASLTIIGLQPEDEADYYCSAWDTSLSTWVFGGGTKLTVL
(SEQ ID NO: 745)

TABLE 58
Ab_25 Variable Region amino acid sequences
V_{H} chain of Ab_25
QVQLVQSGGGLVQPGGSLRLSCAASGFTFSSYWMSWVRQAPGKGLEWVANIKQD
GSEKYYVDSVKGRFTISRDNAKNSLYLQMNSLRAEDTAVYYCARGFYYYGAFDIW
GQGTTVTVSS
(SEQ ID NO: 746)
V_{L} chain of Ab_25
NFMLTQPHSVSESPGKTVTISCTGSSGSIASNYVQWYQQRPGSAPTTVIYEDNQRPS
GVPDRFSGSIGSSSNSASLTISGLKTEDEADYYCQSYDSSNHWVFGGGTKLTVL
(SEQ ID NO: 747)

TABLE 59
Ab_26 Variable Region amino acid sequences
V_{H} chain of Ab_26
QVQLVQSGGGLVQPGRSLRLSCAASGFTFDDYAMHWVRQVPGKGLEWVSGIDW
NSGVIGYADSVKGRFTISRDNAKNSLYLQMNSLRAEDTALYYCAKDAYSYGFLGA
FDIWGQGTMVTVSS
(SEQ ID NO: 748)
V_{L} chain of Ab_26
LPVLTQPPSVSVAPGQTASITCGGDNIRTKGVHWYQQKPGQAPLLVIYYASDRPSGI
PERFSGSSSGNTATLTISRVEAGDEADYYCQVWDSSSDLVVFGGGTTLTVL
(SEQ ID NO: 749)

TABLE 60
Ab_27 Variable Region amino acid sequences
V_{H} chain of Ab_27
QVQLVQSGTEVKKPGASVKVSCKASGYSFTGSHLHWVRQAPGQGLEWMGWINPD
SGVINYAQKFQGRVTLTRDTSISTAYMELSGLRSDDTAVYYCARDKAIGYVWALDY
WGQGTLVTVSS
(SEQ ID NO: 750)
V_{L} chain of Ab_27
QSALTQPPSVSGSPGQSVTISCTGTSSDVGTYNRVSWYQQPPGKAPKLMIYEVSNRP
SGVSNRFSGSKSGNTASLTISGLQAEDEADYYCSSYTRTFTYVFGTGTKVTVL
(SEQ ID NO: 751)

TABLE 61
Ab_28 Variable Region amino acid sequences
V_{H} chain of Ab_28
QVTLKESGPGLVNPSETLSLTCTVSGASISNSFWSWIRQSPGKGLEWIGYTSYSGNSI
YNPSLKSRLTMSIDTSKNQLSLNLRSLTAADTAVYYCARREWIKGHFDYWGQGTL
VTVSS
(SEQ ID NO: 752)
V_{L} chain of Ab_28
NFMLTQPHSVSESPGKTVTISCTGSGGSIASNYVQWYQQRPGSAPTTVIYEDNQRPS
GVPDRFSGSIDSSSNSASLTISGLKTEDEADYYCQSYDSSNPVVFGGGTKLTVL
(SEQ ID NO: 753)

TABLE 62
Ab_38 Variable Region amino acid sequences
V_{H} chain of Ab_38
QVTLKESGPTLVKPTQTLTLTCTFSGFSLTTSGVSVGWIRQPPGKALEWLALIHWD
DDKRYSPSLRSRLTITRDTSKNQVVLTVTDMDPADTGTYYCASFIMTVYAEYFED
WGQGTLVTVSS
(SEQ ID NO: 981)
V_{L} chain of Ab_38
EIVLTQSPATLSVSPGERATLSCRASQSVSSNLAWYQQKPGQAPRLLMYDVSTRAT
GIPARFSGSGSGTEFSLTISSLQSEDFAVYYCQQRGAWPLTFGGGTKVEIK
(SEQ ID NO: 982)

TABLE 65
wcS2-T4-E7 Variable Region amino acid sequences
V_{H} chain of T4-E7
EVQLVQSGGGVVQPGRSLRLSCAASGFTFSSYAMHWVRQAPGKGLEWVAVISYD
GSNKYYADSVKGRFTISRDNSKNTLYLQMNSLRAEDTAVYYCARVNSGSYYGAFD
IWGQGTTVTVSS
(SEQ ID NO: 997)
V_{L} chain of T4-E7
NFMLTQPHSVSESPGKTVTMSCTRSSGDIATRHVQWYQQRPGSAPTTVIYESNQRP
SGVSGRFSGSIDSSSNSASLTISGLQPEDEADYYCQSYDSTNPWVFGGGTKLTVL
(SEQ ID NO: 998)

TABLE 66
wcS2-E1-A9 Variable Region amino acid sequences
V_{H} chain of E1-A9
QVQLV*SGGGLVQPGRSLRLSCAASGFTFDDYAMHWVRQAPGKGLEWVSGISWN
SGSIGYADSVKGRFTISRDNAKNSLYLQMDSLRAEDTAVYYCARDGGIQLSSFEYW
GQGTLVTVSS
(SEQ ID NO: 999)
V_{L} chain of E1-A9
QPVLTQPPSASGSPGQSVTISCTGTSSDVGAYNYVSWYQQHPDKAPKLLIYEVSKRP
SGVPDRFSGSKSGNTASLTVSGLQADDEADYYCSSYAGTRKYYVFGGGTKVTVL
(SEQ ID NO: 1000)

TABLE 67
wcS2-T4-H8 Variable Region amino acid sequences
V_{H} chain of T4-H8
QV*LVQSGGGVVQPGRSLRLSCAASGFTFSSYGMHWVRQAPGKGLEWVAVISYD
GSNKYYADSVKGRFTISRDNSKNTLYLQMNSLRAEDTAVYYCARGSGSYLTDFDY
WGQGTLVTVSS
(SEQ ID NO: 1001)
V_{L} chain of T4-H8
ETTLTQSPATLSLSPGERATLSCRASQIVTNNNLAWYQQKPGQAPRLLIYGASSRAT
GIPDRFSGSGSGTDFTLTISRLEPEDFAVYYCQQYYYWPLSFGGGTKVEIK
(SEQ ID NO: 1002)

TABLE 68
wcS2-T3-F5 Variable Region amino acid sequences
V_{H} chain of T3-F5
QVQLVQSGGGVVQPGRSLRLSCAASGFTFSSYAMHWVRQAPGKGLEWVAVMSY
DGSNKYYADSVKGRFTISRDNAKNSLYLQMNSLRDEDTAVYYCARTGGYLRPIDY
WGQGTLVTVSS
(SEQ ID NO: 1003)
V_{L} chain of T3-F5
QPVLTQPRSVSGSPGQSVTISCTGTSSDVGGYKYVSWYQHHPGKAPKLMIYDVSER
PSGVSSRFSGSKSGNTASLTISGLQGEDEADYFCSSFSQTNSYVFGTGTRVAVL
(SEQ ID NO: 1004)

TABLE 69
wcS2-E2-C1 Variable Region amino acid sequences
V_{H} chain of E2-C1
EVQLVQSGGGVVQPGRSLRLSCAASGFTFSSYAMHWVRQAPGKGLEWVAVISYD
GSNKYYADSVKGRFTISRDNSKNTLYLQMNSLRAEDTAVYYCARDRSGSYWGAF
DIWGQGTLVTVSS
(SEQ ID NO: 1005)
V_{L} chain of E2-C1
QSVLTQPPSASGSPGQSVTISCTGTSSDVGRYKYVSWYQQHPGKAPKPMIYEVNKR
PSGVPDRFSGSKSGNTASLTVSGLQAEDEADYYCSSYAGSNNPYVFGTGTKVTVL
(SEQ ID NO: 1006)

TABLE 70
wcS2-T4-C9 Variable Region amino acid sequences
V_{H} chain of T4-C9
QVQLVQSGGGVVQPGRSLRLSCAASGFTFSSYGMHWVRQAPGKGLEWVAVISYD
GSNKYYADSVKGRFTISRDNSKNTLYLQMNSLRAEDTAVYYCAKGRGSYSTYFDY
WGQGTLVTVSS
(SEQ ID NO: 1007)
V_{L} chain of T4-C9
EIVLTQSPATLSLSPGERATLSCRASQNVPSNSLAWYQQKPGQAPRLLINGASSRAN
GIPDRFSGSGSGTDFTLTITRLEPEDFAVYFCQLYDRSSQLAFGGGTKLEIK
(SEQ ID NO: 1008)

TABLE 71
wcS2-T3-B9 Variable Region amino acid sequences
V_{H} chain of T3-B9
QVQLVQSGGGVVQPGRSLRLSCAASGFTFSGYAMHWVRQAPGKGLEWVAVVSY
DGSNKYYADSVKGRFTISRDNSKNTLYLQMNSLRAEDTAVYYCARSEGYSSGWPL
DYWGQGTLVTVSS
(SEQ ID NO: 1009)
V_{L} chain of T3-B9
SSELTQDPAVSVALGQTVRITCQGDSLGTYYASWYQQKPGQAPLLVIYGKNNRPS
GIPDRFSGSSSGNTASLTITGAQAEDEADYYCNSRNNSGYHEFGGGTKLTVL
(SEQ ID NO: 1010)

TABLE 72
wcS2-E3-C8 Variable Region amino acid sequences
V_{H} chain of E3-C8
QV*LVQSGAEVKKPGESLKISCKGSGYNFIDYWIAWVRQLPGQGLEWMGIIYPGDS
DARYSPSFQGQVTISADKSINTAYLQWSRLKASDTAKYYCARGYAMDVWGQGTTV
TVSS
(SEQ ID NO: 1011)
V_{L} chain of E3-C8
QSVLTQPLSASGTPGHRVTISCSGSQSNIGSNTVNWYQQVPGTAPKLLIYVNNRRPS
GVPDRFSGSKSGTSAALAISGLQSEDEADYYCSSYAGSNDYVFGTGTKVTVL
(SEQ ID NO: 1012)

TABLE 73
wcS2-T4-F8 Variable Region amino acid sequences

V_{H} chain of T4-F8
QVELVQSGAEVKEPGASVKVSCKDSGHTFLGHYMHWVRQAPGQGLEWMG
WINPNSGVTKYAEKFQGWVTMTRDTSISTAYMELSRLKSDDTALYYCAR
ERTTGGAFDIWGQGTMVTVSS
(SEQ ID NO: 1013)
V_{L} chain of T4-F8
NFMLTQPHSVSESPGKTVTISCTRSSGSIASNYVQWYQQRPGSAPTAVI
FGDNQRPSGVPDRFSGSIDSSSNSASLTISGLRTEDEADYYCQSFDGSY
HWVFGGGTKLTVL
(SEQ ID NO: 1014)

TABLE 74
wcS2-T3-F1 Variable Region amino acid sequences

V_{H} chain of T3-F1
EVQLVQSGGGVVQPGRSLRLSCAASGFTFSSYAMHWVRQAPGKGLEWVA
VISYDGSNKYYADSVKGRFTISRDNSKNTLYLQMNSLRAEDTAVYYCAR
DRSGSYWGAFDIWGQGTLVTVSS
(SEQ ID NO: 1015)
V_{L} chain of T3-F1
LPVLTQPPSASGTPGQRVTISCSGSSSNIGSNTVNWYQQLPGTAPKLLI
YSNNQRPSGVPDRFSGSKSGTSASLAISGLRSEDEADYYCATWDDGLSG
RVFGGGTNLAVL
(SEQ ID NO: 1016)

TABLE 75
wcS2-E2-B1 Variable Region amino acid sequences

V_{H} chain of E2-B1
QVQLVQSGGGVVQPGRSLRLSCAASGFTFSSYAMHWVRQAPGKGLEWVA
VISYDGSNKYYADSVKGRFTISRDNSKNTLYLQMNSLRAEDTAVYYCAR
GDYYYYMDVWGKGTLVTVSS
(SEQ ID NO: 1017)
V_{L} chain of E2-B1
SSELTQDPAVSVALGQTVRITCQGDSLRSYYASWYQQKPGQAPVLVIYG
KNNRPSGIPDRFSGSSSGNTASLTITGAQAEDEADYYCNSRDSSGNHLR
VFGTGTKVTVL
(SEQ ID NO: 1018)

TABLE 76
wcS2-T2-G3 Variable Region amino acid sequences

V_{H} chain of T2-G3
QVQLQESGGGVVQPGRSLRLSCAASGFTFSSYAMHWVRQAPGKGLEWVA
VISYDGSNKYYADSVKGRFTISRDNSKNTLYLQMNSLRAEDTAVYYCAR
GLGGGYYYGMDVWGQGTLVTVSS
(SEQ ID NO: 1019)
V_{L} chain of T2-G3
SYELTQPPSVSQGLRQTATLTCTGNSNNVGNQGAAWLQQHQGHPPKLLS
YRNNIRPSGISERLSASTSGNTASLTITGLQPEDEADYYCSAWDNSLGA
WVFGEGTKLTVL
(SEQ ID NO: 1020)

TABLE 77
wcS2-T1-A6 Variable Region amino acid sequences

V_{H} chain of T1-A6
QV*LVQSGAEVKKPGESLKISCKGSGYNFIDYWIAWVRQLPGQGLEWMG
IIYPGDSDARYSPSFQGQVTISADKSINTAYLQWSRLKASDTAKYYCAR
G*AMDVWGQGTTVTVSS
(SEQ ID NO: 1021)
V_{L} chain of T1-A6
QSVLTQPLSASGTPGHRVTISCSGSQSNIGSNTVNWYQQVPGTAPKLLI
YVNNRRPSGVPDRFSGSKSGTSAALAISGLQSEDEADYYCSSYAGSNDY
VFGTGTKVTVL
(SEQ ID NO: 1022)

TABLE 78
wcS2-T4-D4 Variable Region amino acid sequences

V_{H} chain of T4-D4
EV*LVQSGGGVVQPGRSLRLSCAASGFTFSSYAMHWVRQAPGKGLEWVA
VISYDGSNKYYADSVKGRFTISRDNSKNTLYLQMNSLRAEDTAVYYCAR
GNRGSYYGAFDSWGQGTTATASS
(SEQ ID NO: 1023)
V_{L} chain of T4-D4
NFMLTHPHSVS*SPGKTATMSCTRSSGDIATRHVQWYQKRPGSAPTTVI
YESNQRPAGVSGRFSGSIDSSSNSASLTISAVHPEDEADYYCLTYDITN
PWVFGGGTNLTVL
(SEQ ID NO: 1024)

TABLE 79
wcS2-T2-D10 Variable Region amino acid sequences

V_{H} chain of T2-D10
EVQLVQSGGGVVQPGRSLRLSCAASGFTFSSYAMHWVRQAPGKGLEWVA
VISYDGSNKYYADSVKGRFTISRDNSKNTLYLQMNSLRAEDTAVYYCAR
DNSGSYYGAFDIWGQGTLVTVSS
(SEQ ID NO: 1025)
V_{L} chain of T2-D10
HFVLTQPPSASESPGKTVTMSCTGTSGDIATKHDDCCHQRPPGAPPTAM
NDNDHKPSAGSADLFAAFDSASTSALIAFSVLHADDDDDYCCSYDDSTN
PCVFGAGTKVTVL
(SEQ ID NO: 1026)

TABLE 80
wcS2-T1-G9 Variable Region amino acid sequences

V_{H} chain of T1-G9
QVQLVQSGGGVVQPGRSLRLSCAASGFTFSSYGMHWVRQAPGKGLEWVA
VISYDGSNKYYADSVKGRFTISRDNSKNTLYLQMNSLRAEDTAVYYCAK
GRGSYSTYFDYWGQGTLVTVSS
(SEQ ID NO: 1027)
V_{L} chain of T1-G9
ETTLTQSPGTLSLSPGERATLSCRASQSISSSYLAWYQQKPGQAPRLLI
YDASNRAPGIPARFSGSGSGTDFTLTISSLEPEDFAVYYCQQRGNWPLT
FGPGTKVHIK
(SEQ ID NO: 1028)

TABLE 81
wcS2-E3-H7 Variable Region amino acid sequences

V_{H} chain of E3-H7
QVQLVQSGGGVVQPGRSLRLSCAASGFTFSIYGMHWVRQAPGKGLEWVA
GISYDGSNKYYADSVKGRFTISRDNSKNTLYLQMNSLRAEDTAVYYCAK
GRGSYSTYFDYWGQGTLVTVSS
(SEQ ID NO: 1029)
V_{L} chain of E3-H7
EIVLTQSPATLALSPGERATLSCRDSQNVPSNSLAWYQQRPGQAPRLLI
NGASSRANGIPYRFSGSGSGTDFTLTITRLEPEDFAVYFCQLYDRSSQL
ASAERTKMEIK
(SEQ ID NO: 1030)

TABLE 82
wcS2-T2-C11 Variable Region amino acid sequences

V_{H} chain of T2-C11
QVTLKESGPTLVKPTQTLTLTCTFSGFSLSTSGVGVGWIRQPPGKALEW
LALIYWDDDKRYSPSLKSRLTISKDTSRNQVVLTMTNMDPADTGTYYCA
HRRPDWDAFDVWGQGTMVTVSS
(SEQ ID NO: 1031)
V_{L} chain of T2-C11
SSELTQDPAVSVALGQTVRITCQGDSLTIFFANWYQQKPGQAPILVMSK
DTERPSGIPERFSGSSSGTRVTLTISGVQAEDEADYYCQSADTSGTLKV
FGGGTKLTVL
(SEQ ID NO: 1032)

TABLE 83
SARS2-R3-G2-P1-H9_PelB-F_2020-06-19_D08
Variable Region amino acid sequences

V_{H} chain of G2-P1-H9
QVQLVQSGGGLVKPGGSLRLSCSASGFTFSSYAMHWVRQAPGKGLEWVA
VISYDGSNKYYADSVQGRITISRDNSKNTLYLQMNSLRAEDTAVYFCAR
SDGYPYEPFDYWGQGTLVTVSS
(SEQ ID NO: 1033)
V_{L} chain of G2-P1-H9
QSALTQPASVSGSPGQSITISCTGTSSDLGGHNFVSWYQQHPGKAPKLM
IYGVNKRPSGVPDRFSGSKSGNTASLTVSGLQAEDEADYYCSSYEATHI
YVFGTGTKVAVL
(SEQ ID NO: 1034)

TABLE 84
SARS2-R3-G3-P1-F8_PelB-F_2020-06-19_F11
Variable Region amino acid sequences

V_{H} chain of G3-P1-F8
QVQLVQSGGGVVQPGRSLRLSCAASGFTFSSYGMHWVRQAPGKGLEWVA
VISYDGSNKYYADSVKGRFTISRDNSKNTLYLQMNSLRAEDTAVYYCAR
GSGITGAFRDWGQGTLVTVSS
(SEQ ID NO: 1035)
V_{L} chain of G3-P1-F8
QSALTQPASVSGSPGQSITISCTGTSSDVGSYNLVSWYQQHPGKAPKLM
ISEVSNRPSGVSNRFSGSKSGNTASLTISGLQAEDEADYYCSSFSSGSI
PYVFGAGTKVTVL
(SEQ ID NO: 1036)

TABLE 85
SARS2-R3-G2-P1-B4_PelB-F_2020-06-19_B02
Variable Region amino acid sequences

V_{H} chain of G2-P1-B4
QVQLVQSGGGVVQPGRSLRLSCAASGFTFSSYGMHWVRQAPGKGLEWVA
VISYDGSNKYYADSVKGRFTISRDNSKNTLYLQMNSLRTEDTAVYYCAR
PQGGAYNGYFDSWGQGTLVTVSS
(SEQ ID NO: 1037)
V_{L} chain of G2-P1-B4
EIVLTQSPATLSVSPGERATLSCRASQSVSTNLAWYQQKPGQAPRLLIY
GASTRATGIPARFSGSGSGTEFTLTVSRLEPEDFAVYYCQHYANWPRTF
GQGTRVEIR
(SEQ ID NO: 1038)

TABLE 86
SARS2-R3-G3-P1-B2 PelB-F 2020-06-19_A08
Variable Region amino acid sequences

V_{H} chain of G3-P1-B2
QVQLVQSGGGVVQPGRSLRLSCAASGFTFSSYAMHWVRQAPGKGLEWVA
VISYDGSNKYYADSVKGRFTISRDNSKNTLYLQMNSLRAEDTAVYYCAR
SDSGNYYGAFDIWGQGTTVTVSS
(SEQ ID NO: 1039)
V_{L} chain of G3-P1-B2
QSVLTQPPSASGTPGQRVTISCSGSSSNIGSNTVNWYQQLPGTAPKLLI
YSNNQRPSGVPDRFSGSKSGTSVSLAISGLQSEDEADYYCATWDDSLNG
PVFGGGTNLAVL
(SEQ ID NO: 1040)

TABLE 87
SARS2-R3-G3-P1-B3_PelB-F_2020-06-19_B08
Variable Region amino acid sequences

V_{H} chain of G3-P1-B3
QVQLQESGGGVVQPGRALRLSCRASGFNFGTFGMHWVRQAPGKGLEWVA
VISYDGSNKYYADSVKGRFTISRDNSKNTLYLQMNSLRAEDTAVYYCAR
VRGSGYPHAFDIWGQGTMVTVSS
(SEQ ID NO: 1041)
V_{L} chain of G3-P1-B3
SYELTQPPSVSVAPGKTANMTCGGNNIGSKSVHWYQQKPGQAPVLVVYD
DTDRPPGIPERFSGSNSGNTATLTISRVEVGDEADYYCQVWDTSSDHPY
VFGTGTKVTVL
(SEQ ID NO: 1042)

TABLE 88
SARS2-R3-G1-P3-E8_PelB-F_2020-06-19_H04 Variable Region amino acid
sequences

V_{H} chain of G1-P3-E8
*VQLVQSGAEVKKPGSSVKVSCKASGGTFSSYAISWVRQAPGQGLEWMGGIIPIFG
TANYAQKFQGRVTITADKSTSTAYMELSSLRSEDTAVYYCARYKWEPNVGGAFDI
WGQGTMVTVSS
(SEQ ID NO: 1043)
V_{L} chain of G1-P3-E8
SSELTQDPAVSVALGQTVRITCQGDSLRSYYASWYQQKPGQAPVLVIYGGNNRPS
GIPDRISGSSSGNTASLTITGAQAEDEADYYCSSRATSAFYVFGTGTKVTVL
(SEQ ID NO: 1044)

TABLE 89
SARS2-R3-G1-P2-D7_PelB-F_2020-06-19_H09 Variable Region amino acid
sequences

V_{H} chain of G1-P2-D7
EVQLVQSGAEVKKPGASVKVSCKASGYTFTSYGISWVRQAPGQGLEWMGWISAY
NGNTNYAQKLQGRVTMTTNTSTNTAYMELRSLRSDDTAGYYWAKGGSYGRYGM
DVWGQGTTVTVSS
(SEQ ID NO: 1045)
V_{L} chain of G1-P2-D7
QSGLTQPPSVSGAPGQRVTISCTGSSSNIGAGYDVHWYQQLPGTAPKLIIYGNSNRP
SGVPDRFSGSKSGTSASLAITGLQAEDEADYYCQSYDSSLSGWVFGGGTKLTVL
(SEQ ID NO: 1046)

TABLE 90
SARS2-R3-G1-P1-C6_PelB-F_2020-06-19_H02 Variable Region amino acid
sequences

V_{H} chain of G1-P1-C6
QVTLKESGPTLVKPTQTLTLTCTFSGFSLSTSGVGVGWIRQPPGKALEWLALIYWD
DDKRYSPSLKSRLTITKDTSKNQVVLTMTNMDPVDTATYYCAKMGNAWTFEHWG
QGTLVTVSS
(SEQ ID NO: 1047)
V_{L} chain of G1-P1-C6
SYELTQPPSVSVSPGQTARITCSGDALPNRYAYWYQQRPGQAPVLVIYKDSERPSGI
PERFSGSSSGTTVTLTISGVQAEDEADYYCQSVNTIGTYVFGGGTKVTVL
(SEQ ID NO: 1048)

TABLE 91
SARS2-R3-T-P2-E12_PelB-F_2020-06-19_D07 Variable Region amino acid
sequences

V_{H} chain of T-P2-E12
QVQLVQSGGGLVQPGGSLRLSCSASGFTFSSYAMHWVRQAPGKGLEWVEVISYDG
SNKYYAYSVQGRFTISKNNSKNTLYLQMNSLKAEDTAVYFCARANGCPY*PFYYR
GRGTPGTASS
(SEQ ID NO: 1049)
V_{L} chain of T-P2-E12
HTVLTHPAPAAAYPGQTITISCSATSSDLVGHKEVSWYQQHPGKAPTLVIYEINNR
PSGVPDRFSGSIYGNTDSLTVSAGVEDEDDYYCTCYDEDHIYVS*TGTEVAFI
(SEQ ID NO: 1050)

TABLE 92
SARS2-R3-G2-P1-D12_PelB-F_2020-06-19_H04 Variable Region amino acid
sequences

V_{H} chain of G2-P1-D12
QVQLVQSGGGVVQPGRSLRLSCAASGFIFSSYGMHWVRQAPGKGLEWVAVISYD
GSNKYYADSVKGRFTISRDNSKNTLYLQMNSLRVEDTAVYYCARCSGGSCYFNG
MDVWGQGTMVTVSS
(SEQ ID NO: 1051)
V_{L} chain of G2-P1-D12
DIVMTQTPSTLSASVGDRVTISCRASENVNNWVAWYQQKPGKVPELLMYKASRLE
PGVPSRFSGSGSGTEFTLTISNLQPEDFATYYCQQYKSYSFGQGTKVDIK
(SEQ ID NO: 1052)

TABLE 93
SARS2-R3-G1-P2-E8_PelB-F 2020-06-19_E10 Variable Region amino acid
sequences

V_{H} chain of G1-P2-E8
EVQLVQSGGGVVQPGRSLRLSCAASGFTFSSYAMHWVRQAPGKGLEWVAVISYD
GSNKYYADSVKGRFTISRDNSKNTLYLQMNSLRAEDTAVYYCARDRSGSYWGAF
DIWGQGTLVTVSS
(SEQ ID NO: 1053)
V_{L} chain of G1-P2-E8
NFMLTQPHSVSESPGKTVTISCTRSSGSIASNYVQWYQQRPGSSPTAVIYAHNQRPS
EVPDRFSGSIDISSNSASLTISGLKTEDEADYYCHSYDTNNPVVFGGGTKLTVL
(SEQ ID NO: 1054)

TABLE 94
SARS2-R3-G1-P1-A10_PelB-F_2020-06-19_H01 Variable Region amino acid
sequences

V_{H} chain of G1-P1-A10
EVQLVQSGGGVVQPGRSLTLSCAASGFTFSSYAMHWVRQAPGKGLEWVAVISYD
GSNKYYADSVKGRFTISRDNSKNTLYLQMNSLRAEDTAVYYCARDRSGSYWGAF
DIWGQGTTVTVSS
(SEQ ID NO: 1055)
V_{L} chain of G1-P1-A10
ETTLTQSPGTLSLSPGERATLSCRASQSVRSNLAWYQQKPGQAPRLLIYGVSTR
ATGIPARFSGSGSGTDFTLTISRLEPEDFAVYFCQHYGSSPLYTFGQGTKLEIK
(SEQ ID NO: 1056)

TABLE 95
SARS2-R3-G1-P3-B5_PelB-F_2020-06-19_D02 Variable Region amino acid
sequences

V_{H} chain of G1-P3-B5
QVQLVQSGGGVVQPGRSLRLSCAASGFTFSSYGMHWVRQAPGKGLEWVALISYD
GSNKYYADSVKGRFTISRDNAKNSLYLQMNSLRAEDTAVYYCARDYGLRYFDYW
GQGTLVTVSS
(SEQ ID NO: 1057)
V_{L} chain of G1-P3-B5
QSALTQPPSASGSPGQSVTISCTGTSSDVGSYNRVSWYQQPPGTAPKLMIYEVSN
RPSGVPDRFSGSKSGNTASLTISGLQAEDEADYYCSSYTSSSPYVFGTGTKVTVL
(SEQ ID NO: 1058)

TABLE 96
SARS2-R3-G3-P1-A2_PelB-F_2020-06-19_B07 Variable Region amino acid
sequences

V_{H} chain of G3-P1-A2
QVQLVQSGTEVRQPGASVKVSCKASGYTFTSYGISWVRQAPGQGLEWMGGIIPIF
GTANYAQKFQGRVTITADESTSTAYMELSSLRSEDTAVYYCARSMVQGVIRFYGM
DVWGQGTTVTVSS
(SEQ ID NO: 1059)
V_{L} chain of G3-P1-A2
NFMLTQPHSVSGSPGKTVTISCTRDIGDIARNYVQWYQQRPGSSPTTVIYEDDRR
PSGVPDRFSGSVDRSSNSASLTISGLDTEDEADYYCQSYDTTIVFGGGTKLTVL
(SEQ ID NO: 1060)

TABLE 97
SARS2-R3-G3-P1-D1_PelB-F 2020-06-19_G09 Variable Region amino acid
sequences

V_{H} chain of G3-P1-D1
EVQLVQSGGGVVQPGRSLRLSCAASGFTFSSYAMHWVRQAPGKGLEWVAVISYD
GSNKYYADSVKGRFTISRDNSKNTLYLQMNSLRAEDTAVYYCARSSNYYYMDVW
GKGTLVTVSS
(SEQ ID NO: 1061)
V_{L} chain of G3-P1-D1
QPVLTQPPSVSGAPGQSVTISCIGSSSNIGAGYHVQWYQQVPGTAPKLLIYGNQNR
PSGVPDRFSGSKSGTSASLAISGLRSEDEADYFCSAWDDSLGGEVFGTGTKVNVL
(SEQ ID NO: 1062)

TABLE 98
SARS2-R3-G1-P2-D4_PelB-F_2020-06-19_G09 Variable Region amino acid
sequences

V_{H} chain of G1-P2-D4
QVQLVQSGGGVVRPGRSLRLSCAASGFTFSSYALHWVRQAPGKGLEWVAVISYDG
SHKYYADSVKGRFTISRDNAKNSLYLQMNSLRAEDTAVYYCARVLGGSYRMGAF
DIWGQGTMVTVSS
(SEQ ID NO: 1063)
V_{L} chain of G1-P2-D4
QPGLTQPPSVSKDLRQTATLTCTGNSNNVGHEGAAWLQQHQGHPPKLLSYKNDNR
PSGISERFSASTSGNTASLTITGLQPEDEADYYCSAWDSSLGSWVFGGGSRLTVL
(SEQ ID NO: 1064)

TABLE 99
SARS2-R3-G1-P1-B6_PelB-F_2020-06-19_E02 Variable Region amino acid
sequences

V_{H} chain of G1-P1-B6
EVQLVQSGGGVVQPGRSLRLSCAASGFTFSSYAMHWVRQAPGKGLEWVAVISYD
GSNKYYADSVKGRFTISRDNSKNTLYLQMNSLRAEDTAIYYCSRDAVAGYRGGFD
YWGQGTLVTVSS
(SEQ ID NO: 1065)
V_{L} chain of G1-P1-B6
QPVLTQPPSASGTPGQRVTISCSGGRSNIGSNTVNWYQQLPGTAPKLLIYSNNHR
PSGVPDRFSGSKSGNTASLTISGLQAEDEADYYCQSYDSSVVFGGGTKLTVL
(SEQ ID NO: 1066)

TABLE 100
SARS2-R3-G3-P1-G8_PelB-F_2020-06-25_A05 Variable Region amino acid
sequences

V_{H} chain of G3-P1-G8
QVTLKESGPALVKPTQTLTLTCTFSGFSLSTSGVGVGWIRQPPGKALEWLALIYWD
DDKRYSPSLKSRLTITKDTSKNQVVLTMTNMDPVDTATYYCAHLDTFYGMDVWG
QGTLVTVSS
(SEQ ID NO: 1067)
V_{L} chain of G3-P1-G8
QPVLTQPPSVSVSPGQTASITCSGDNLGEIYASWYQQKPGQSPVLVIYQDKKRPSG
IPERFSGSNSGNTATLTISETQAMDEADYYCQAWDSSTGVFGTGTKVTVL
(SEQ ID NO: 1068)

TABLE 101
SARS2-R3-G1-P1-F11_PelB-F_2020-06-25_B05 Variable Region amino
acid sequences

V_{H} chain of G1-P1-F11
QVTLKESGPTLVKPTQTLTLTCTLSGFSLTTSGVGVGWIRQPPGKALEWLALIYWD
DDKRYSPSLKSRLTITKDTSKNQVVLTMTNMDPVDTATYYCAHSRGLWGAFDIW
GQGTTVTVSS
(SEQ ID NO: 1069)
V_{L} chain of G1-P1-F11
SYELTQPPSVSVSPGQTATITCSGDALPDKYAYWYRQRPGQAPVLVIYKDSERPSGI
PERFSGSSSGTTVTLTISGVQAEDEADYYCQSADSSGTWVFGGGTKLTVL
(SEQ ID NO: 1070)

TABLE 102
SARS2-R3-T1-P3-F4_PelB-F_2020-06-20_C03 Variable Region amino acid
sequences

V_{H} chain of T1-P3-F4
QVQLVQSGGGVVQPGRSRRLSCTASGITFYSYGMHWVRQAPGKGLEWVSTISNIY
NTHYADSVKGRFTISRDNSKNTLYLQMKSLRAEDTATYYCAKGIAAADYWGQGTL
VTVSS
(SEQ ID NO: 1071)
V_{L} chain of T1-P3-F4
QSVLTQPPSASGSPGQSVTISCTGTSSDVGGYNHVSWYQQHPGKAPKVLIYDVSKR
PSGVPDRFSGSKSGNTASLTVSGLQAEDEADYYCTSYAGSNSLVFGTGTEVTVL
(SEQ ID NO: 1072)

TABLE 103
SARS2-R3-G1-P2-B8_PelB-F_2020-06-25_C06 Variable Region amino acid
sequences
V_{H} chain of G1-P2-B8
QVQLVQSGGGLVQPGGSLRLSCAASGFTFSSYAMSWVRQAPGKGLEWVSAISGSG
GSTYYADSVKGRFTISRDNSKNTLYLQMNSLRAEDTGVYYCANDIQGDPVRWGQ
GTLVTVSS
(SEQ ID NO: 1073)
V_{L} chain of G1-P2-B8
QSVLTQPPSASGSPGHSVTISCTGTSGDVGGYNSVSWYQHHPGKAPKLMIYEVTKR
PSGVPDRFSGSKSGNTASLSVSGLQAEDEADYYCSSYAGSNNYVFGTGTKVTVL
(SEQ ID NO: 1074)

TABLE 104
SARS2-R3-G1-P2-D3_PelB-F_2020-06-25_C12 Variable Region amino acid
sequences
V_{H} chain of G1-P2-D3
QVQLVQSGGGVVQPGRSLRLSCAASGFTFSSYAMHWVRQAPGKGLEWVAVISYD
GSNKYYADSVKGRFTISRDNSKNTLYLQMNSLRAEDTAVYYCARARGYSYGHHF
DYWGQGTLVTVSS
(SEQ ID NO: 1075)
V_{L} chain of G1-P2-D3
NFMLTQPHSVSESPGKTVAISCTRSSGSIASNYVQWYQQRPGSSPTTVIYEDNQRPS
GVPDRFSGSIDSSSNSASLTISGLKTEDEADYYCQSYDSPWVFGGGTKLTVL
(SEQ ID NO: 1076)

TABLE 105
SARS2-R3-G1-P4-C5_PelB-F_2020-06-25_C07 Variable Region amino acid
sequences
V_{H} chain of G1-P4-C5
EVQLVQSGGGVVQPGRSLRLSCAASGFTFSSYGMHWVRQAPGKGLEWVSYISSSS
KYIYYADSVKGRFTISRDNAKNSLYLQMNSLRAEDTAVYYCARDADTSSSESDYW
GQGTLVTVSS
(SEQ ID NO: 1077)
V_{L} chain of G1-P4-C5
LPMLTQPPSMSGTPGQRVTISCSGSSSNIGSNTVNWYQQVPGTAPKVLIYSNNQRPS
GVPDRFSGSKSGTSASLAISGLRSEDEADYSCAVWDDSLNGLVIGGGTKLTVL
(SEQ ID NO: 1078)

TABLE 106
SARS2-R3-G1-P2-D6_PelB-F_2020-06-25_D02 Variable Region amino acid
sequences
V_{H} chain of G1-P2-D6
QVQLVQSGAEVKKPGASVKVSCKASGYTFSSYAFSWVRQAPGQGLEWMGWISAF
NGNTDYAQNFQGRVTMTTDTSTNTAYMELRSLRSDDTAVYYCARDGGKLDYWG
QGTLVTVSS
(SEQ ID NO: 1079)
V_{L} chain of G1-P2-D6
SYELTQPPSVSKGLRQTATLTCTGNSNNVGHEGASWLQHHQGHPPKLLSYRNKNR
PSGISERFSASRSGNTASLTITGLQPEDEADYYCSAWDGRLNGYLFGTGTKVTVL
(SEQ ID NO: 1080)

TABLE 107
SARS2-R3-G1-P1-B7_PelB-F_2020-06-25_A03 Variable Region amino acid
sequences
V_{H} chain of G1-P1-B7
EVQLVQSGAEVKKPGASVKVSCKASGYTFTSYYMHWVRQAPGQGLEWMGIINPS
GGSTSYAQKFQGRVTMTRDTSTSTVYMELSSLRSEDTAVYYCARDLPLRYRPNAF
DIWGQGTTVTVSS
(SEQ ID NO: 1081)
V_{L} chain of G1-P1-B7
SYELTQPPSVSVSPGQTATITCSGDKLEDKFVSWYQQKPGHSPLLVIYEDAKRPSGIP
ERFSGSNSGNRAILTINGTQALDEADYYCQAWAITTEVFGGGTKLAVL
(SEQ ID NO: 1082)

TABLE 108
SARS2_95_PelB-F_2020-06-25_G11 Variable Region amino acid sequences
V_{H} chain of G11
QVQLVQSGGGVVQPGRSLRLSCAASGFTFSNYAMHWVRQAPGKGLEWVAVISYD
GSTEYYADSVKGRFTISRDNSKKMLYLQMNSLTAEDTAVYYCARDPRYCSGAGCY
YYGMDVWGQGTTVTVSS
(SEQ ID NO: 1083)
V_{L} chain of G11
ETTLTQSPGTLSLSPGERATLSCRASQSVSSTYLAWYQQKPGQAPRLLIYGASNRAT
GIPDRFSGSGSGTDFTLTISSLQPEDFATYYCQHSYRTPYTFGQGTKLEIN
(SEQ ID NO: 1084)

TABLE 109
SARS2_73_PelB-F_2020-06-25_F09 Variable Region amino acid sequences
V_{H} chain of F09
QVQLVQSGGGLVQPGGSLRLSCAASGFTFSSYEMNWVRQAPGKGLEWVAVISYD
GSNKYYADSVKGRFTISRDNSKNTLYLQMNSLRAEDTAVYYCARDPSYGLDYWG
QGTLVTVSS
(SEQ ID NO: 1085)
V_{L} chain of F09
DIVMTQSPATLSVSPGERATLSCRASQSVRDGYLAWYQQRPGQAPRLLISGASTRA
TDIPDRFTGSGSGTDFTLTISSLEPEDFAVYYCQQRSNWPPTFGQGTKVESK
(SEQ ID NO: 1086)

TABLE 110
SARS2-R3-G3-P1-G9_PelB-F_2020-06-25_B05 Variable Region amino acid
sequences
V_{H} chain of G3-P1-G9
QVQLVQSGGGLVKPGGSLRLSCAASGFTFSSYAMHWVRQAPGKGLEWVAVISYD
GSNKYYADSVKGRFTISRDNSKNTLYLQMNSLRAEDTAVYYCARDRGGYSGYLDY
WGQGTLVTVSS
(SEQ ID NO: 1087)
V_{L} chain of G3-P1-G9
QSVLTQPPSASGSPGQSVTVSCTGTSDDVGGFAHVSWYQQHPGKAPKLLIHDVSKR
PSGVPDRFSASKSGNTASLTISGLQPEDEGDYFCSSYAGRNGVIFGGGTTLTVL
(SEQ ID NO: 1088)

TABLE 111
SARS2-R3-G1-P3-G7_PelB-F_2020-06-25_H01 Variable Region amino acid
sequences
V_{H} chain of G1-P3-G7
QVQLVQSGAEVKKPGASVKVSCKASGYTFTGYYMHWVRQAPGQGLEWMGRINP
NSGGTNYAQKFQGRVTMTRDTSISTAYMELSRLRSDDTAVYYCARESGLLWFGDY
YYGMDVWGQGTTVTVSS
(SEQ ID NO: 1089)
V_{L} chain of G1-P3-G7
QPVLTQPPSVSKDLRQTATLTCTGNSNNVGDQGTAWLQQHQGHPPKLVSYRNNN
RPSGVSERFSASRSGNTASLTITGLQAEDEADYYCSAWDSSLSAQVFGGGTKLTVL
(SEQ ID NO: 1090)

TABLE 112
SARS2-R3-G1-P4-A2_PelB-F_2020-06-25_A06 Variable Region amino acid
sequences
V_{H} chain of G1-P4-A2
QVQLQQSGAEVKKPGSSVKVSCKASGSTFNNYAVSWVRQAPGQGPEWMGRIIPIV
DIANYAQRFQGRVTITADESTNTAYMELSSLRSEDTAVYYCARGGSQGAYYMDVW
GKGTTVTVSS
(SEQ ID NO: 1091)
V_{L} chain of G1-P4-A2
NFMLTQPHSVSESPGKTVTISCTRSSGSIASNYVQWYQQRPGSAPTTVIYEDNQRPS
GVPDRFSGSIDSSSKTASLIISGLETEDEADYYCQSYDNNNHVVFGGGTKLTVP
(SEQ ID NO: 1092)

TABLE 113
SARS2-R3-G3-P1-G1_PelB-F_2020-06-25_F04 Variable Region amino acid
sequences
V_{H} chain of G3-P1-G1
EVQLVQSGAEVKKPGASVKVSCKASGYTFTSYGISWVRQAPGQGLEWMGWISAY
NGNTNYAQKLQGRVTMTTDTSTSTAYMELRSLRSDDTAVYYCARGGSYGRYGMD
VWGQGTTVTVSS
(SEQ ID NO: 1093)
V_{L} chain of G3-P1-G1
QSVLTQPPSVSGAPGQRVTISCTGSSSNIGAGYDVHWYQQLPGTAPKLIIYGNSNRP
SGVPDRFSGSKSGTSASLAITGLQAEDEADYYCQSYDSSLSGWVFGGGTKLTVL
(SEQ ID NO: 1094)

TABLE 114
SARS2-R3-G3-P1-F1_PelB-F_2020-06-25_C04 Variable Region amino acid
sequences
V_{H} chain of G3-P1-F1
QVQLVQSGGGVVQPGRSLRLSCAASGFTFSSYAMHWVRQAPGKGLEWVAVISYD
GSNKYYADSVKGRFTISRDNSKNTLYLQMNSLRDEDTAVYYCARGLSGTYRATTF
DFWGQGTLVTVSS
(SEQ ID NO: 1095)
V_{L} chain of G3-P1-F1
QSALTQPASVSGSPGQSITISCTGTSSDLGGHNFVSWYQQHPASAPKLIIYDVYNRPS
GVSNRFSGSESGDTASLTISGLRAEDEADYFCSSYAGSNPYVFGTGTKVTVL
(SEQ ID NO: 1096)

TABLE 115
SARS2-R3-G1-P3-H10_PelB-F_2020-06-25_H10 Variable Region amino
acid sequences
V_{H} chain of G1-P3-H10
QVQLQESGAGLLRPSETLSLTCAVYGGSFSSYHWSWIRQPPGKGLEWIGEIDHYGS
PNYNPSFQSRVAMSRDTPKNQFSLKLSSVTAADTAVYYCARGYAFDIWGQGTLVT
VSS
(SEQ ID NO: 1097)
V_{L} chain of G1-P3-H10
LPVLTQPPSASETPGQRVTISCSGGRSNIGINSVNWYQQLPGTAPKLLIYRNNQRPSG
VPDRFFGSKSGTSASLAISGLQSEDEADYYCAAWDGSLNGPLFGGGTKLTVL
(SEQ ID NO: 1098)

TABLE 116
SARS2-85_PelB-F_2020-06-25_G10 Variable Region amino acid sequences
V_{H} chain of G10
QVQLV*SGAEVKKPGESLKISCQGSGYNFNDYWVGWVRQKPGKGLEWMGIIYPG
DSDTRKNPSFEGQVTMSVDKSLHSVYLHWTSLKVSDTAKYYCARGYAMDVWGQG
TLGTVSS
(SEQ ID NO: 1099)
V_{L} chain of G10
QPVLTQPPSVSGAPGQSVTISCTGSSSNIGNYGVHWYQQLPGTAPKLLIYSNNQRPS
GVPDRFSGSKSGTSASLAISGLQSEDEADYYCAAWDDSLKGVFGGGTKVTVL
(SEQ ID NO: 1100)

TABLE 117
SARS2-R3-G1-P4-A3_PelB-F_2020-06-25_B06 Variable Region amino acid
sequences
V_{H} chain of G1-P4-A3
QVQLVQSGAEVKKPGASVKVSCKASGYTFSNYDINWVRQATGQGLEWMGWMNP
NSGNTGYAQSFQGRVTFTRDTSINTAYMELSSLRSEDTAVYYCARGYGMDVWGQ
GTTVTVSS
(SEQ ID NO: 1101)
V_{L} chain of G1-P4-A3
QSVLTQPPSASGTPGGRVTISCSGSTSNIGRNKVYWYQRLPGTAPKLLIYLNNQRPS
GVPDRFSGSKSGTSASLAISGLQSEDEADYFCAAWDDSLTGYVFGTGTKVTVL
(SEQ ID NO: 1102)

TABLE 118
SARS2-R3-G1-P3-H6_PelB-F_2020-06-25_H08 Variable Region amino
acid sequences
V_{H} chain of G1-P3-H6
QVQLVQSGAEVKKSGESLEISCKGSGYSFTNYWISWVRQMPGKGLEWMGRIDPRD
SYTNYSPSFQGHVTITVDKSTGAAYLHWSSLKASDTGMYYCARHPAGGTSFDMW
GQGTMVTVSS
(SEQ ID NO: 1103)
V_{L} chain of G1-P3-H6
SSELTQDPAVSVALGQTVRIICQGDSLSRYYANWYQQKSGQAPILVMYGKDIRPSIP
DRFSGSSSGNTASLTITGAQEDEADYYCNSRDSSGNLVRRRIKLTVL
(SEQ ID NO: 1104)

TABLE 119
SARS2-R3-G3-P1-G5_PelB-F_2020-06-25_H04 Variable Region amino
acid sequences
V_{H} chain of G3-P1-G5
EVQLVQSGAEVKKPGESLRISCKGSGYTFTNYWIGWVRQMPGKGLEWMGVIYPG
DSDTIYSPSFQGQVTISADKSISTAYLQWSSLKASDTAIYYCARRAGAQGAFDIWGQ
GTMVTVSS
(SEQ ID NO: 1105)
V_{L} chain of G3-P1-G5
QSALTQPASVSGSPGQSITISCTGTSSDVGGYNYVSWYQQHPGKAPKLMIYDVNYR
PSGISHRFSGSKSGNTASLTISGLQAEDEADYYCSSYTSSSTYVFGTGTKVTVL
(SEQ ID NO: 1106)

TABLE 120
SARS2-R3-G1-P4-C10_PelB-F_2020-06-25_F07 Variable Region amino
acid sequences
V_{H} chain of G1-P4-C10
EVQLVESGGGQVKPGGSLRISCAASGFTFENYNMHWVRQAPGKGLEWVSSITGHA
YYTYYADSLKGRFNISRDNAKKLLYLQLSSLSAEDTALYFCARSNYASAGHGMDV
WGHGTLVTVSS
(SEQ ID NO: 1107)
V_{L} chain of G1-P4-C10
SYELTQPPSVSVSPGQTASITCSGDGLPKHYAYWYQQRPGQAPVLLIYKDTERSSGI
PERFSGSGSGTTVTLTINGVQAEDEADYYCQSGDTSGPVVFGGGTKLTVL
(SEQ ID NO: 1108)

TABLE 121
SARS2-R3-G1-P1-F10_PelB-F_2020-06-25_B04 Variable Region amino
acid sequences
V_{H} chain of G1-P1-F10
QVQLVQSGAEVKKPGSSVKVSCKASGYTFTSYGISWVRQAPGQGLELMGWISAYN
GNTNYAQKLQGRVTMTTDTSTSTAYMELRSLRSGDTAVYYCARTVGSLDYWGQG
TLVTVSS
(SEQ ID NO: 1109)
V_{L} chain of G1-P1-F10
LPVLTQPPSVSGAPGQRVAISCTGSSSNIGRGYNVHWYQQLPGAAPKLLIYGNSNRP
SGVPDRFSGSKSGTSASLAISGLRSEDEADYYCSAWDDSLNNVVFGGGTRLTVL
(SEQ ID NO: 1110)

TABLE 122
SARS2-R3-T1-P4-H4_PelB-F_2020-06-20_H04 Variable Region amino
acid sequences
V_{H} chain of T1-P4-H4
EVQLVESGPGLVKPSQTLSLTCTVSGGSISSGGYYWSWIRQHPGKGLEWIGYIYYS
GSTYYNPSLKSRVTISVDTSKNQFSLKLSSVTAADTAVYYCARYDRDAFDIWGQGT
MVTVSS
(SEQ ID NO: 1111)
V_{L} chain of T1-P4-H4
QSVLTQPPSVSVAPGQTARITCGGDNIGHKGVHWYQQKAGQAPVLVVHDDSDRPS
GIPERFSGSNSGNTATLTISRVEAGDEADYYCQVWESYGDHVVFGGGTKLTVL
(SEQ ID NO: 1112)

TABLE 123
SARS2-R3-G2-P1-E8_PelB-F_2020-06-25_A02 Variable Region amino
acid sequences
V_{H} chain of G2-P1-E8
QVQLVQSGGGVVRPGRSLRLSCAASGFTFSSYAMHWVRQAPGKGLEWVAVISYD
GSNKYYADSVKGRFTISRDNAKNSLYLQMNSLRAEDTAVYYCASGSYAQFDYWG
QGTLVTVSS
(SEQ ID NO: 1111)
V_{L} chain of G2-P1-E8
DIVMTQSPLSLPVTPGEPASISCRSSQSLLHSNGYNYLDWYLQKPGQSPQLLIHFGS
NRASGVPDRFSGSGSDTDFTLKISRVEAEDVGVYYCMQVLQTPPITFGQGTRLDIK
(SEQ ID NO: 1112)

TABLE 124
SARS2-R3-G1-P1-C3_PelB-F_2020-06-25_A07 Variable Region amino
acid sequences
V_{H} chain of G1-P1-C3
QVQLVQSGGGLVQPGGSLRLSCAASGFTFSSYGMHWVRQAPGKGLEWVAVISYD
GSNKYYADSVKGRFTISRDNSKNTLYLQMNSLRAEDTAVYYCATPLGFFGAFDIW
GQGTLVTVSS
(SEQ ID NO: 1113)
V_{L} chain of G1-P1-C3
QSVLTQPPSVSGAPGERVTFSCTGTTSNIGAGYDVHWYQQLPGTAPKLLIYDNNIRP
SGVPDRFSASKSGTSASLAITGLQSEDEGDYYCQSYDSGLDGSVFGGGTKLTVL
(SEQ ID NO: 1114)

TABLE 125
SARS2-R3-G3-P1-G2_PelB-F_2020-06-25_G04 Variable Region amino
acid sequences
V_{H} chain of G3-P1-G2
EVQLVQSGGGLVQPGGSLRLPCSASGFDFSNYDMHWVRQAPGKGLEHISIITRDGG
RTDYAESVKGRFTISRDNSKNTLYLQMTSLREEDTAVYYCDKAGGEWGQGTLVTV
SS
(SEQ ID NO: 1115)
V_{L} chain of G3-P1-G2
QSVLTQPPSASGSPGQSVTISCTGTSSDVGGYNHVSWYQQHPGKAPKVLIYDVSKR
PSGVPDRFSGSKSGNTASLTVSGLQAEDEADYYCTSYAGSNSLVFGTGTEVTVL
(SEQ ID NO: 1116)

TABLE 126
SARS2-R3-G1-P4-D1_PelB-F_2020-06-25_A08 Variable Region amino
acisequences
V_{H} chain of G1-P4-D1
QVQLVQSGAEVKKPGSSAKVSCKASGGTFNNYAISWVRQAPGQGPEWMGRIIPIV
DIANYAQRFQGRVTITADESTNTAYMELSSLRSEDTAVYYCVRGGSQGAYYMDVW
GKGTLVTVSS
(SEQ ID NO: 1117)
V_{L} chain of G1-P4-D1
NFMLTQPHSVSESPGKTVTISCTRSSGSIASNYVQWYQQRPGSAPTTVIYEDDQRPS
GVPDRFSASIDSSSNSASLTISGLKAEDEADYYCQSYDNNNAVIFGGGTKLTVL
(SEQ ID NO: 1118)

The amino acid sequences of the heavy and light chain complementary determining regions of the anti-SARS CoV antibodies are shown in Table 63A-B below:

TABLE 63A
Heavy chain (V_{H}) complementary determining regions (CDRs) of the COVID-
19 antibodies.

| Sequence | | | |
|---|---|---|---|
| ID | CDR1-IMGT | CDR2-IMGT | CDR3-IMGT |
| S1-R3-T1- | GGSIRTHS | IHHSGAT | ARGPGILSY |
| H7 (Ab 12) | (SEQ ID NO: 93) | (SEQ ID NO: 94) | (SEQ ID NO: 95) |
| RBD-R3- | GGSISSYY | IYTSGST | ARDVGFGWFDR |
| E1-G7 | (SEQ ID NO: 96) | (SEQ ID NO: 97) | (SEQ ID NO: 98) |
| (Ab2-10) | | | |
| S1-RBD- | GGSIRTHS | IHHSGAT | ARGPGILSY |
| R3-E1-D8 | (SEQ ID NO: 99) | (SEQ ID NO: 100) | (SEQ ID NO: 101) |
| (Ab_3) | | | |
| S1-RBD- | GDSVSSYSDA | TYYRSKWYN | AREIVATTPFRNYYYGMDV |
| R3-T1-C7 | (SEQ ID NO: 102) | (SEQ ID NO: 103) | (SEQ ID NO: 104) |
| RBD-R3- | GFTFSHYD | IGYDGTNL | ARAANYYDSSGYGRADAF |
| T1-F4 | (SEQ ID NO: 105) | (SEQ ID NO: 106) | DI |
| (Ab2-2) | | | (SEQ ID NO: 107) |
| RBD-R3- | GFTFSDFP | ISYDGNIK | ARGGSSFDI |
| E1-A5 | (SEQ ID NO: 108) | (SEQ ID NO: 109) | (SEQ ID NO: 110) |
| S1-RBD- | GFSLSTTGVG | IYWNDDK | ARISGSGYFYPFDI |
| R3-T1-F5 | (SEQ ID NO: 111) | (SEQ ID NO: 112) | (SEQ ID NO: 113) |
| (Ab2-7) | | | |
| S1-R3-T1- | GFTFTTYG | ISYDGSIK | ARVGDSSSYYGIDA |
| A12 (Ab_5) | (SEQ ID NO: 114) | (SEQ ID NO: 115) | (SEQ ID NO: 116) |
| S1-R3-T1- | GFTFSSHA | ISYDGSYT | ARDWVNFGMDV |
| A6 (Ab_4) | (SEQ ID NO: 117) | (SEQ ID NO: 118) | (SEQ ID NO: 119) |
| S1-RBD- | GYTFSDYY | IDPNSGGT | ARDRGRGGQAGAFDY |
| R3-T1-A5 | (SEQ ID NO: 120) | (SEQ ID NO: 121) | (SEQ ID NO: 978) |
| S1-RBD- | GFTFSSYA | ISYGGSNK | AKVRGSGWYWGSAFDI |
| R3-T1-B3 | (SEQ ID NO: 122) | (SEQ ID NO: 123) | (SEQ ID NO: 124) |
| (Ab_1) | | | |
| S1-RBD- | GYSFTGSH | INPDSGVI | ARDKAIGYVWALDY |
| R3-T1-E7 | (SEQ ID NO: 125) | (SEQ ID NO: 126) | (SEQ ID NO: 127) |
| S1-RBD- | GVSLDTIGMR | IDWDDDK | ARSGLLYDLDV |
| R3-T1-F9 | (SEQ ID NO: 128) | (SEQ ID NO: 129) | (SEQ ID NO: 130) |
| S1-R3-T1- | GFTFSDYP | TSYDGRIK | ARDPGWLRSVGMDV |
| C2 (Ab_6) | (SEQ ID NO: 131) | (SEQ ID NO: 132) | (SEQ ID NO: 133) |
| RBD-R3- | GYSFTSYW | IYPGDSDT | ARGWQWHDY |
| E1-B3 | (SEQ ID NO: 134) | (SEQ ID NO: 135) | (SEQ ID NO: 136) |
| RBD-R3- | GDSVSSRSSA | TYYRSNWNY | VRNMRPDFDL |
| T1-B5 | (SEQ ID NO: 137) | (SEQ ID NO: 138) | (SEQ ID NO: 139) |
| RBD-R3- | GYTFTTSG | ISAYNGNT | ARDFHLYYGMDV |
| T1-H3 | (SEQ ID NO: 140) | (SEQ ID NO: 141) | (SEQ ID NO: 142) |
| (Ab_2) | | | |
| S1-R3-T1- | GGTFSSYA | INPNSGGT | ARGSGGYYLG |
| C4 (Ab_8) | (SEQ ID NO: 143) | (SEQ ID NO: 144) | (SEQ ID NO: 145) |
| S1-RBD- | GGTFSSYT | IIPILGTP | AVGSGWYSGFDY |
| R3-T1-B12 | (SEQ ID NO: 146) | (SEQ ID NO: 147) | (SEQ ID NO: 148) |
| S1-RBD- | GFTFSSYW | IKQDGSEK | ARGFYYYGAFDI |
| R3-T1-G5 | (SEQ ID NO: 149) | (SEQ ID NO: 150) | (SEQ ID NO: 151) |
| S1-RBD- | GFTFDDYA | IDWNSGVI | AKDAYSYGFLGAFDI |
| R3-T1-E2 | (SEQ ID NO: 152) | (SEQ ID NO: 153) | (SEQ ID NO: 154) |
| (Ab7) | | | |
| RBD-R3- | GFTFDDYA | IDWNSGVI | ARDILPSNFDGKKIIVFQPPA |
| T1-F7 | (SEQ ID NO: 155) | (SEQ ID NO: 156) | KRDLDNYYGMDV |
| | | | (SEQ ID NO: 157) |
| S1-RBD- | GYSFTSNW | IFPGDSDT | ARESYNAYGS |
| R3-T1-G1 | (SEQ ID NO: 158) | (SEQ ID NO: 159) | (SEQ ID NO: 160) |
| S1-RBD- | GYTFTSYG | ISAYNGNT | ARGFPQLGSDY |
| R3-T1-C2 | (SEQ ID NO: 161) | (SEQ ID NO: 162) | (SEQ ID NO: 163) |
| S1-RBD- | GGTFSSYA | ISGYNGNT | ARQMKDSGNYWEYYYYG |
| R3-T1-H8 | (SEQ ID NO: 164) | (SEQ ID NO: 165) | MDV |
| | | | (SEQ ID NO: 166) |
| S1-RBD- | GYTFTSYG | ISTYNGNT | ARDVFGHFDY |
| R3-E1-E8 | (SEQ ID NO: 167) | (SEQ ID NO: 168) | (SEQ ID NO: 169) |
| RBD-R3- | GFSLTTTGVS | IHWDDDK | ASFIMTVY AEYFED |
| T1-H2 | (SEQ ID NO: 170) | (SEQ ID NO: 171) | (SEQ ID NO: 172) |
| S1-RBD- | GFSLSTSAMC | IDWDNDR | AHSPYDSIWGSFRPSVYYF |
| R3-T1-B7 | (SEQ ID NO: 173) | (SEQ ID NO: 174) | DY |
| | | | (SEQ ID NO: 175) |
| S1-RBD- | GFTFSDYY | ISSSSSDT | AMPTREPAY |
| R3-E1-E5 | (SEQ ID NO: 176) | (SEQ ID NO: 177) | (SEQ ID NO: 178) |
| S1-R3-T1- | GFAFSDFP | ISYDGSLK | AREGVSNSRPFDH |
| H6 | (SEQ ID NO: 179) | (SEQ ID NO: 180) | (SEQ ID NO: 181) |
| S1-RBD- | GFTFSSYA | ISSNGGST | TRDLWSGSADSFDI |
| R3-E1-C6 | (SEQ ID NO: 182) | (SEQ ID NO: 183) | (SEQ ID NO: 184) |
| S1-RBD- | GFPFNAYY | INQDGSEK | ARLYWWGMDV |
| R3-E1-F2 | (SEQ ID NO: 185) | (SEQ ID NO: 186) | (SEQ ID NO: 187) |
| S1-RBD- | GFTFDDYA | IDWNSGVI | AKDAYSYGFLGAFDI |
| R3-T1-C3 | (SEQ ID NO: 188) | (SEQ ID NO: 189) | (SEQ ID NO: 190) |
| S1-RBD- | GFTFDDYA | ISWNSGSI | ARDWWGSIDH |
| R3-T1-G12 | (SEQ ID NO: 191) | (SEQ ID NO: 192) | (SEQ ID NO: 193) |
| S1-RBD- | GGSISSSNW | IYHSGST | ARRGGTYHRGAFDI |
| R3-E1-F1 | (SEQ ID NO: 194) | (SEQ ID NO: 195) | (SEQ ID NO: 196) |
| S1-RBD- | GASISNSF | TSYSGNS | ARREWIKGHFDY |
| R3-E1-H8 | (SEQ ID NO: 197) | (SEQ ID NO: 198) | (SEQ ID NO: 199) |
| S1-RBD- | GGSFTTHS | ILPGGAT | ARGPGILSY |
| R3-T1-F1 | (SEQ ID NO: 200) | (SEQ ID NO: 201) | (SEQ ID NO: 202) |
| S1-R3-T1- | GGSFRTHS | IHHSGAT | ARGPGILSY |
| B10 | (SEQ ID NO: 203) | (SEQ ID NO: 204) | (SEQ ID NO: 205) |
| RBD-R3- | GGSIRTHS | IHHSGAT | ARGPGILSY |
| E1-D12 | (SEQ ID NO: 206) | (SEQ ID NO: 207) | (SEQ ID NO: 208) |
| RBD-R3- | GGSIRTHS | IHHSGAT | GRGPGILSY |
| T1-C5 | (SEQ ID NO: 209) | (SEQ ID NO: 210) | (SEQ ID NO: 211) |
| S1-RBD- | GYSFTSYW | IYPGDSDT | ARQGDGGGYDY |
| R3-T1-B4 | (SEQ ID NO: 212) | (SEQ ID NO: 213) | (SEQ ID NO: 214) |
| S1-RBD- | RYSFSNYW | IYPYDSDT | ARQGSSQSFDI |
| R3-E1-E7 | (SEQ ID NO: 215) | (SEQ ID NO: 216) | (SEQ ID NO: 217) |
| S1-RBD- | GYSFTSYW | IYPGDSDT | ARRRGSAAAFDT |
| R3-T1-C8 | (SEQ ID NO: 218) | (SEQ ID NO: 219) | (SEQ ID NO: 220) |
| S1-RBD- | GYSFTSYW | IYPGDSDT | ARTTYSYGSFDY |
| R3-T1-D7 | (SEQ ID NO: 221) | (SEQ ID NO: 222) | (SEQ ID NO: 223) |
| RBD-R3- | GDSVTSNSAA | TYYSSKWYN | ARGWLRLSFDP |
| E1-F5 | (SEQ ID NO: 224) | (SEQ ID NO: 225) | (SEQ ID NO: 226) |
| Ab_13 | GFSLSTSGVG | IYWDDDK | ARISGSGYFYPFDI |
| | (SEQ ID NO: 754) | (SEQ ID NO:755) | (SEQ ID NO: 756) |
| Ab_14 | GDSVSSNSAA | TYYRSRWYN | AREIRGFDY |
| | (SEQ ID NO: 757) | (SEQ ID NO: 758) | (SEQ ID NO: 759) |
| Ab_15 | GFTFGDYA | IRSKAYGGTT | TTADDDMDV |
| | (SEQ ID NO: 760) | (SEQ ID NO: 761) | (SEQ ID NO: 762) |
| Ab_16 | GFTFSNYG | IWERGSKK | AREGISMTGAEYFQH |
| | (SEQ ID NO: 763) | (SEQ ID NO: 764) | (SEQ ID NO: 765) |
| Ab_17 | GFTFDDYA | IDWNSGVI | AKDIGPGGSGSYY AFDI |
| | (SEQ ID NO: 766) | (SEQ ID NO: 767) | (SEQ ID NO: 768) |
| Ab_18 | GFSFSRYG | IRHDGSKK | AKDGRLEAALDD |
| | (SEQ ID NO: 769) | (SEQ ID NO: 770) | (SEQ ID NO: 771) |
| Ab_19 | GYSFTSYW | IYPGDSDT | ARRGDLDAFDI |
| | (SEQ ID NO: 772) | (SEQ ID NO: 773) | (SEQ ID NO: 774) |
| Ab_20 | GYRLSDYY | IKQDGSEK | ARVRGWSRGYFDY |
| | (SEQ ID NO: 775) | (SEQ ID NO: 776) | (SEQ ID NO: 777) |
| Ab_21 | GFTFDDYA | ISWNSGSI | ARDWWGSIDH |
| | (SEQ ID NO: 778) | (SEQ ID NO: 779) | (SEQ ID NO: 780) |
| Ab_22 | GFTFSHYD | IGYDGTNL | ARAANYYDSSGYGRADAF |
| | (SEQ ID NO: 781) | (SEQ ID NO: 782) | DI (SEQ ID NO: 783) |
| Ab_23 | GGTFSTYG | IIPSLGIP | ARENIDLATNDF |
| | (SEQ ID NO: 784) | (SEQ ID NO: 785) | (SEQ ID NO: 786) |
| Ab_24 | GGTFSSSG | IIPMLGTP | ARDGGNYDY |
| | (SEQ ID NO: 787) | (SEQ ID NO: 788) | (SEQ ID NO: 789) |
| Ab_25 | GFTFSSYW | IKQDGSEK | ARGFYYYGAFDI |
| | (SEQ ID NO: 790) | (SEQ ID NO: 791) | (SEQ ID NO: 792) |
| Ab_26 | GFTFDDYA | IDWNSGVI | AKDAYSYGFLGAFDI |
| | (SEQ ID NO: 793) | (SEQ ID NO: 794) | (SEQ ID NO: 795) |
| Ab_27 | GYSFTGSH | INPDSGVI | ARDKAIGYVWALDY |
| | (SEQ ID NO: 796) | (SEQ ID NO: 797) | (SEQ ID NO: 798) |
| Ab_28 | GASISNSF | TSYSGNS | ARREWIKGHFDY |
| | (SEQ ID NO: 799) | (SEQ ID NO: 800) | (SEQ ID NO: 801) |
| Ab_38 | GFSLTTSGVS | IHWDDDK | ASFIMTVYAEYFED |
| | (SEQ ID NO: 983) | (SEQ ID NO: 984) | (SEQ ID NO: 985) |
| wCS2-T4-E7 | GFTFSSYA | ISYDGSNK | ARVNSGSYYGAFDI |
| | (SEQ ID NO: 122) | (SEQ ID NO: ) | (SEQ ID NO: ) |
| wcS2-E1-A9 | GFTFDDYA | ISWNSGSI | ARDGGIQLSSFEY |
| | (SEQ ID NO: 152) | (SEQ ID NO: 192) | (SEQ ID NO: ) |
| wCS2-T4-H8 | GFTFSSYG | ISYDGSNK | ARGSGSYLTDFDY |
| | (SEQ ID NO:) | (SEQ ID NO: ) | (SEQ ID NO: ) |
| wCS2-T3-F5 | GFTFSSYA | MSYDGSNK | ARTGGYLRPIDY |
| | (SEQ ID NO: 122) | (SEQ ID NO: ) | (SEQ ID NO: ) |
| wCS2-E2-C1 | GFTFSSYA | ISYDGSNK | ARDRSGSYWGAFDI |
| | (SEQ ID NO: 122) | (SEQ ID NO: ) | (SEQ ID NO: ) |
| wCS2-T4-C9 | GFTFSSYG | ISYDGSNK | AKGRGSYSTYFDY |
| | (SEQ ID NO: ) | (SEQ ID NO: ) | (SEQ ID NO: ) |
| wCS2-T3-B9 | GFTFSGYA | VSYDGSNK | ARSEGYSSGWPLDY |
| | (SEQ ID NO: ) | (SEQ ID NO: ) | (SEQ ID NO: ) |
| wcS2-E3-C8 | GYNFIDYW | IYPGDSDA | ARGYAMDV |
| | (SEQ ID NO: ) | (SEQ ID NO: ) | (SEQ ID NO: ) |
| wCS2-T4-F8 | GHTFLGHY | INPNSGVT | ARERTTGGAFDI |
| | (SEQ ID NO: ) | (SEQ ID NO: ) | (SEQ ID NO: ) |
| wCS2-T3-F1 | GFTFSSYA | ISYDGSNK | ARDRSGSYWGAFDI |
| | (SEQ ID NO: 122) | (SEQ ID NO: ) | (SEQ ID NO: ) |
| wcS2-E2-B1 | GFTFSSYA | ISYDGSNK | ARGDYYYYMDV |
| | (SEQ ID NO: 122) | (SEQ ID NO: ) | (SEQ ID NO: ) |
| wCS2-T2-G3 | GFTFSSYA | ISYDGSNK | ARGLGGGYYYGMDV |
| | (SEQ ID NO: 122) | (SEQ ID NO: ) | (SEQ ID NO: ) |
| wCS2-T1-A6 | GYNFIDYW | IYPGDSDA | ARG*AMDV |
| | (SEQ ID NO: ) | (SEQ ID NO: ) | (SEQ ID NO: ) |
| wcS2-T4-D4 | GFTFSSYA | ISYDGSNK | ARGNRGSYYGAFDS |
| | (SEQ ID NO: 122) | (SEQ ID NO: ) | (SEQ ID NO: ) |
| wCS2-T2- | GFTFSSYA | ISYDGSNK | ARDNSGSYYGAFDI |
| D10 | (SEQ ID NO: 122) | (SEQ ID NO: ) | (SEQ ID NO: ) |
| wCS2-T1-G9 | GFTFSSYG | ISYDGSNK | AKGRGSYSTYFDY |
| | (SEQ ID NO: ) | (SEQ ID NO: ) | (SEQ ID NO: ) |
| wcS2-E3-H7 | GFTFSIYG | ISYDGSNK | AKGRGSYSTYFDY |
| | (SEQ ID NO: ) | (SEQ ID NO: ) | (SEQ ID NO: ) |
| wCS2-T2- | GFSLSTSGVG | IYWDDDK | AHRRPDWDAFDV |
| C11 | (SEQ ID NO: ) | (SEQ ID NO: ) | (SEQ ID NO: ) |
| SARS2-R3- | GFTFSSYA | ISYDGSNK | ARSDGYPYEPFDY |
| G2-P1- | (SEQ ID NO: ) | (SEQ ID NO: ) | (SEQ ID NO: ) |
| H9_PelB- | | | |
| F_2020-06- | | | |
| 19_D08 | | | |
| SARS2-R3- | GFTFSSYG | ISYDGSNK | ARGSGITGAFRD |
| G3-P1- | (SEQ ID NO: ) | (SEQ ID NO: ) | (SEQ ID NO: ) |
| F8_PelB- | | | |
| F_2020-06- | | | |
| 19_F11 | | | |
| SARS2-R3- | GFTFSSYG | ISYDGSNK | ARPQGGAYNGYFDS |
| G2-P1- | (SEQ ID NO: ) | (SEQ ID NO: ) | (SEQ ID NO: ) |
| B4_PelB- | | | |
| F_2020-06- | | | |
| 19_B02 | | | |
| SARS2-R3- | GFTFSSYA | ISYDGSNK | ARSDSGNYYGAFDI |
| G3-P1- | | | |
| B2_PelB- | | | |
| F_2020-06- | | | |
| 19_A08 | | | |
| SARS2-R3- | GFNFGTFG | ISYDGSNK | ARVRGSGYPHAFDI |
| G3-P1- | | | |
| B3_PelB- | | | |
| F_2020-06- | | | |
| 19_B08 | | | |
| SARS2-R3- | GGTFSSYA | IIPIFGTA | ARYKWEPNVGGAFDI |
| G1-P3- | | | |
| E8_PelB- | | | |
| F_2020-06- | | | |
| 19_H04 | | | |
| SARS2-R3- | GYTFTSYG | ISAYNGNT | AKGGSYGRYGMDV |
| G1-P2- | | | |
| D7_PelB- | | | |
| F_2020-06- | | | |
| 19_H09 | | | |
| SARS2-R3- | GFSLSTSGVG | IYWDDDK | AKMGNAWTFEH |
| G1-P1- | | | |
| C6_PelB- | | | |
| F_2020-06- | | | |
| 19_H02 | | | |
| SARS2-R3- | GFTFSSYA | ISYDGSNK | ARANGCPY*PFYY |
| T-P2- | | | |
| E12_PelB- | | | |
| F_2020-06- | | | |
| 19_D07 | | | |
| SARS2-R3- | GFIFSSYG | ISYDGSNK | ARCSGGSCYFNGMDV |
| G2-P1- | | | |
| D12_PelB- | | | |
| F_2020-06- | | | |
| 19_H04 | | | |
| SARS2-R3- | GFTFSSYA | ISYDGSNK | ARDRSGSYWGAFDI |
| G1-P2- | | | |
| E8_PelB- | | | |
| F_2020-06- | | | |
| 19_E10 | | | |
| SARS2-R3- | GFTFSSYA | ISYDGSNK | ARDRSGSYWGAFDI |
| G1-P1- | | | |
| A10_PelB- | | | |
| F_2020-06- | | | |
| 19_H01 | | | |
| SARS2-R3- | GFTFSSYG | ISYDGSNK | ARDYGLRYFDY |
| G1-P3- | | | |
| B5_PelB- | | | |
| F_2020-06- | | | |
| 19_D02 | | | |
| SARS2-R3- | GYTFTSYG | IIPIFGTA | ARSMVQGVIRFYGMDV |
| G3-P1- | | | |
| A2_PelB- | | | |
| F_2020-06- | | | |
| 19_B07 | | | |
| SARS2-R3- | GFTFSSYA | ISYDGSNK | ARSSNYYYMDV |
| G3-P1- | | | |
| D1_PelB- | | | |
| F_2020-06- | | | |
| 19_G09 | | | |
| SARS2-R3- | GFTFSSYA | ISYDGSHK | ARVLGGSYRMGAFDI |
| G1-P2- | | | |
| D4_PelB- | | | |
| F_2020-06- | | | |
| 19_G09 | | | |
| SARS2-R3- | GFTFSSYA | ISYDGSNK | SRDAVAGYRGGFDY |
| G1-P1- | | | |
| B6_PelB- | | | |
| F_2020-06- | | | |
| 19_E02 | | | |
| SARS2-R3- | GFSLSTSGVG | IYWDDDK | AHLDTFYGMDV |
| G3-P1- | | | |
| G8_PelB- | | | |
| F_2020-06- | | | |
| 25_A05 | | | |
| SARS2-R3- | GFSLTTSGVG | IYWDDDK | AHSRGLWGAFDI |
| G1-P1- | | | |
| F11_PelB- | | | |
| F_2020-06- | | | |
| 25_B05 | | | |
| SARS2-R3- | GITFYSYG | ISNIYNT | AKGIAAADY |
| T1-P3- | | | |
| F4_PelB- | | | |
| F_2020-06- | | | |
| 20_C03 | | | |
| SARS2-R3- | GFTFSSYA | ISGSGGST | ANDIQGDPVR |
| G1-P2- | | | |
| B8_PelB- | | | |
| F_2020-06- | | | |
| 25_C06 | | | |
| SARS2-R3- | GFTFSSYA | ISYDGSNK | ARARGYSYGHHFDY |
| G1-P2- | | | |
| D3_PelB- | | | |
| F_2020-06- | | | |
| 25_C12 | | | |
| SARS2-R3- | GFTFSSYG | ISSSSKYI | ARDADTSSSESDY |
| G1-P4- | | | |
| C5_PelB- | | | |
| F_2020-06- | | | |
| 25_C07 | | | |
| SARS2-R3- | GYTFSSYA | ISAFNGNT | ARDGGKLDY |
| G1-P2- | | | |
| D6_PelB- | | | |
| F_2020-06- | | | |
| 25_D02 | | | |
| SARS2-R3- | GYTFTSYY | INPSGGST | ARDLPLRYRPNAFDI |
| G1-P1- | | | |
| B7_PelB- | | | |
| F_2020-06- | | | |
| 25_A03 | | | |
| 95_PelB- | GFTFSNYA | ISYDGSTE | ARDPRYCSGAGCYYYGMD |
| F_2020-06- | | | V |
| 25_G11 | | | |
| 73_PelB- | GFTFSSYE | ISYDGSNK | ARDPSYGLDY |
| F_2020-06- | | | |
| 25_F09 | | | |
| SARS2-R3- | GFTFSSYA | ISYDGSNK | ARDRGGYSGYLDY |
| G3-P1- | | | |
| G9_PelB- | | | |
| F_2020-06- | | | |
| 25_B05 | | | |
| SARS2-R3- | GYTFTGYY | INPNSGGT | ARESGLLWFGDYYYGMDV |
| G1-P3- | | | |
| G7_PelB- | | | |
| F_2020-06- | | | |
| 25_H01 | | | |
| SARS2-R3- | GSTFNNYA | IIPIVDIA | ARGGSQGAYYMDV |
| G1-P4- | | | |
| A2_PelB- | | | |
| F_2020-06- | | | |
| 25_A06 | | | |
| SARS2-R3- | GYTFTSYG | ISAYNGNT | ARGGSYGRYGMDV |
| G3-P1- | | | |
| G1_PelB- | | | |
| F_2020-06- | | | |
| 25_F04 | | | |
| SARS2-R3- | GFTFSSYA | ISYDGSNK | ARGLSGTYRATTFDF |
| G3-P1- | | | |
| F1_PelB- | | | |
| F_2020-06- | | | |
| 25_C04 | | | |
| SARS2-R3- | GGSFSSYH | IDHYGSP | ARGYAFDI |
| G1-P3- | | | |
| H10_PelB- | | | |
| F_2020-06- | | | |
| 25_H10 | | | |
| 85_PelB- | GYNFNDYW | IYPGDSDT | ARGYAMDV |
| F_2020-06- | | | |
| 25_G10 | | | |
| SARS2-R3- | GYTFSNYD | MNPNSGNT | ARGYGMDV |
| G1-P4- | | | |
| A3_PelB- | | | |
| F_2020-06- | | | |
| 25_B06 | | | |
| SARS2-R3- | GYSFTNYW | IDPRDSYT | ARHPAGGTSFDM |
| G1-P3- | | | |
| H6_PelB- | | | |
| F_2020-06- | | | |
| 25_H08 | | | |
| SARS2-R3- | GYTFTNYW | IYPGDSDT | ARRAGAQGAFDI |
| G3-P1- | | | |
| G5_PelB- | | | |
| F_2020-06- | | | |
| 25_H04 | | | |
| SARS2-R3- | GFTFENYN | ITGHAYYT | ARSNYASAGHGMDV |
| G1-P4- | | | |
| C10_PelB- | | | |
| F_2020-06- | | | |
| 25_F07 | | | |
| SARS2-R3- | GYTFTSYG | ISAYNGNT | ARTVGSLDY |
| G1-P1- | | | |
| F10_PelB- | | | |
| F_2020-06- | | | |
| 25_B04 | | | |
| SARS2-R3- | GGSISSGGYY | IYYSGST | ARYDRDAFDI |
| T1-P4- | | | |
| H4_PelB- | | | |
| F_2020-06- | | | |
| 20_H04 | | | |
| SARS2-R3- | GFTFSSYA | ISYDGSNK | ASGSYAQFDY |
| G2-P1- | | | |
| E8_PelB- | | | |
| F_2020-06- | | | |
| 25_A02 | | | |
| SARS2-R3- | GFTFSSYG | ISYDGSNK | ATPLGFFGAFDI |
| G1-P1- | | | |
| C3_PelB- | | | |
| F_2020-06- | | | |
| 25_A07 | | | |
| SARS2-R3- | GFDFSNYD | ITRDGGRT | DKAGGE |
| G3-P1- | | | |
| G2_PelB- | | | |
| F_2020-06- | | | |
| 25_G04 | | | |
| SARS2-R3- | GGTFNNYA | IIPIVDIA | VRGGSQGAYYMDV |
| G1-P4- | | | |
| D1_PelB- | | | |
| F_2020-06- | | | |
| 25_A08 | | | |

TABLE 63B
Light chain (V_{L}) complementary determining regions (CDRs) of the COVID-19
antibodies

| Sequence ID | CDR1-IMGT | CDR2-IMGT | CDR3-IMGT |
|---|---|---|---|
| S1-R3-T1-H7 | SSNIGSNT | SNN | AAWDDSLNVHYV |
| (Ab12) | (SEQ ID NO: 227) | (SEQ ID NO: 228) | (SEQ ID NO: 229) |
| RBD-R3-E1- | SGSIASNY | EDN | QSFDSASLWV |
| G7 (Ab2-10) | (SEQ ID NO: 230) | (SEQ ID NO: 231) | (SEQ ID NO: 232) |
| S1-RBD-R3- | SSNIGSND | SNN | ATWDDSLSAGV |
| E1-D8 (Ab_3) | (SEQ ID NO: 233) | (SEQ ID NO: 234) | (SEQ ID NO: 235) |
| S1-RBD-R3- | SGSIASNY | QDK | QSYDSSSLWV |
| T1-C7 | (SEQ ID NO: 236) | (SEQ ID NO: 237) | (SEQ ID NO: 238) |
| RBD-R3-T1- | TGSIAGNY | DDN | QSYDSGNRGV |
| F4 (Ab2-2) | (SEQ ID NO: 239) | (SEQ ID NO: 240) | (SEQ ID NO: 241) |
| RBD-R3-E1- | TSNIGNNA | YNE | AAWDDSLSGHVV |
| A5 | (SEQ ID NO: 242) | (SEQ ID NO: 243) | (SEQ ID NO: 244) |
| S1-RBD-R3- | SGSIASNY | EDN | QSYDSSSLWV |
| T1-F5 | (SEQ ID NO: 245) | (SEQ ID NO: 246) | (SEQ ID NO: 247) |
| (Ab2-7) | | | |
| S1-R3-T1- | SSNIGSNS | SNN | AAWDDSLTGYV |
| A12 (Ab_5) | (SEQ ID NO: 248) | (SEQ ID NO: 249) | (SEQ ID NO: 250) |
| S1-R3-T1-A6 | SSDVGGYNY | EVS | AAWDDSLSGPV |
| (Ab_4) | (SEQ ID NO: 251) | (SEQ ID NO: 252) | (SEQ ID NO: 253) |
| S1-RBD-R3- | KIGSKS | DDS | HVWDSSSDQNV |
| T1-A5 | (SEQ ID NO: 254) | (SEQ ID NO: 255) | (SEQ ID NO: 256) |
| S1-RBD-R3- | SLRAYF | GQD | NSRDSGENHLI |
| T1-B3 (Ab_1) | (SEQ ID NO: 257) | (SEQ ID NO: 258) | (SEQ ID NO: 259) |
| S1-RBD-R3- | SSDVGTYNR | EVS | SSYTRTFTYV |
| T1-E7 | (SEQ ID NO: 260) | (SEQ ID NO: 261) | (SEQ ID NO: 262) |
| S1-RBD-R3- | DSDIGANF | RNT | QSYDSSLSAYV |
| T1-F9 | (SEQ ID NO: 263) | (SEQ ID NO: 264) | (SEQ ID NO: 265) |
| S1-R3-T1-C2 | SGSIARNY | ADR | QSYDSSNQAAV |
| (Ab 6) | (SEQ ID NO: 266) | (SEQ ID NO: 267) | (SEQ ID NO: 268) |
| RBD-R3-E1- | SLRSYY | DKD | NSRDRSDNHVV |
| B3 | (SEQ ID NO: 269) | (SEQ ID NO: 270) | (SEQ ID NO: 271) |
| RBD-R3-T1- | QSVSNN | DAT | QQYDNLPV |
| B5 | (SEQ ID NO: 272) | (SEQ ID NO: 273) | (SEQ ID NO: 274) |
| RBD-R3-T1- | SSDVGAYNY | DVT | AVWDDGLNGRVV |
| H3 (Ab_2) | (SEQ ID NO: 275) | (SEQ ID NO: 276) | (SEQ ID NO: 277) |
| S1-R3-T1-C4 | SNNVGNQG | MNN | SAWDSSLSRWV |
| (Ab_8) | (SEQ ID NO: 278) | (SEQ ID NO: 279) | (SEQ ID NO: 280) |
| S1-RBD-R3- | SGSIASNY | EDS | QSFHNSNPVI |
| T1-B12 | (SEQ ID NO: 281) | (SEQ ID NO: 282) | (SEQ ID NO: 283) |
| S1-RBD-R3- | SGSIASNY | EDN | QSYDSSNHWV |
| T1-G5 | (SEQ ID NO: 284) | (SEQ ID NO: 285) | (SEQ ID NO: 286) |
| S1-RBD-R3- | NIGSKS | EDR | QVWDGDSDHYV |
| T1-E2 (Ab7) | (SEQ ID NO: 287) | (SEQ ID NO: 288) | (SEQ ID NO: 289) |
| RBD-R3-T1- | SSDVGGYNL | EGS | SSYTITDVVV |
| F7 | (SEQ ID NO: 290) | (SEQ ID NO: 291) | (SEQ ID NO: 292) |
| S1-RBD-R3- | SSNIGSNP | SNN | AAWDDSLSGVV |
| T1-G1 | (SEQ ID NO: 293) | (SEQ ID NO: 294) | (SEQ ID NO: 295) |
| S1-RBD-R3- | SGSIASNY | EDN | QSYDSTNWV |
| T1-C2 | (SEQ ID NO: 296) | (SEQ ID NO: 297) | (SEQ ID NO: 298) |
| S1-RBD-R3- | NIGSES | EDR | QVWNPSGSLQYV |
| T1-H8 | (SEQ ID NO: 299) | (SEQ ID NO: 300) | (SEQ ID NO: 301) |
| S1-RBD-R3- | SGNIATNY | EDN | KSYDDGNHV |
| E1-E8 | (SEQ ID NO: 302) | (SEQ ID NO: 303) | (SEQ ID NO: 304) |
| RBD-R3-T1- | QSVSSN | DVS | QQRGVWPLT |
| H2 | (SEQ ID NO: 305) | (SEQ ID NO: 306) | (SEQ ID NO: 307) |
| S1-RBD-R3- | SGSIVSSY | EHN | QSYDSQNGV |
| T1-B7 | (SEQ ID NO: 308) | (SEQ ID NO: 309) | (SEQ ID NO: 310) |
| S1-RBD-R3- | SSDLGTYNY | DVF | SSYTSSSTYV |
| E1-E5 | (SEQ ID NO: 311) | (SEQ ID NO: 312) | (SEQ ID NO: 313) |
| S1-R3-T1-H6 | SIGTKS | DDD | QVWESDDDDLV |
| | (SEQ ID NO: 314) | (SEQ ID NO: 315) | (SEQ ID NO: 316) |
| S1-RBD-R3- | SLRRYY | GKN | NSRDISDNQWQWI |
| E1-C6 | (SEQ ID NO: 317) | (SEQ ID NO: 318) | (SEQ ID NO: 319) |
| S1-RBD-R3- | SSDVGGYKY | DVN | SSYTGRMNLYV |
| E1-F2 | (SEQ ID NO: 320) | (SEQ ID NO: 321) | (SEQ ID NO: 322) |
| S1-RBD-R3- | NIRTKG | YAS | QVWDSSSDLVV |
| T1-C3 | (SEQ ID NO: 323) | (SEQ ID NO: 324) | (SEQ ID NO: 325) |
| S1-RBD-R3- | SSDVGGYDY | DVS | SSYTSSSPVV |
| T1-G12 | (SEQ ID NO: 326) | (SEQ ID NO: 327) | (SEQ ID NO: 328) |
| S1-RBD-R3- | SRDVGSYDL | EGS | SSYTSSNSLV |
| E1-F1 | (SEQ ID NO: 329) | (SEQ ID NO: 330) | (SEQ ID NO: 331) |
| S1-RBD-R3- | GGSIASNY | EDN | QSYDSSNPVV |
| E1-H8 | (SEQ ID NO: 332) | (SEQ ID NO: 333) | (SEQ ID NO: 334) |
| S1-RBD-R3- | SSIGSND | SNN | AWDDSLSAVV |
| T1-F1 | (SEQ ID NO: 335) | (SEQ ID NO: 336) | (SEQ ID NO: 337) |
| S1-R3-T1- | SSNIGSNT | INN | AEWYDSLNVHYV |
| B10 | (SEQ ID NO: 338) | (SEQ ID NO: 339) | (SEQ ID NO: 340) |
| RBD-R3-E1- | SSNIGSNT | INN | AECYDSLNDHYV |
| D12 | (SEQ ID NO: 341) | (SEQ ID NO: 342) | (SEQ ID NO: 343) |
| RBD-R3-T1- | SSNIGSNT | SNN | AAWDDSLNVHYV |
| C5 | (SEQ ID NO: 344) | (SEQ ID NO: 345) | (SEQ ID NO: 346) |
| S1-RBD-R3- | SSNIGSNP | NNN | AAWDDSLNGL |
| T1-B4 | (SEQ ID NO: 347) | (SEQ ID NO: 348) | (SEQ ID NO: 349) |
| S1-RBD-R3- | SLRSYY | GKN | NSRDSSGDVRV |
| E1-E7 | (SEQ ID NO: 350) | (SEQ ID NO: 351) | (SEQ ID NO: 352) |
| S1-RBD-R3- | SSNIGSNP | DNN | EAWDDSLSGPV |
| T1-C8 | (SEQ ID NO: 353) | (SEQ ID NO: 354) | (SEQ ID NO: 355) |
| S1-RBD-R3- | SSNIGGNS | RNN | AAWDDSLNGWV |
| T1-D7 | (SEQ ID NO: 356) | (SEQ ID NO: 357) | (SEQ ID NO: 358) |
| RBD-R3-E1- | SGSIASNY | EDN | QSYDPNNHGVV |
| F5 | (SEQ ID NO: 359) | (SEQ ID NO: 360) | (SEQ ID NO: 361) |
| Ab_13 | SGSIASNY | EDN | QSYDSSNLWV |
| | (SEQ ID NO: 802) | (SEQ ID NO: 803) | (SEQ ID NO: 804) |
| Ab_14 | SSDVGAYNF | DFN | SSYAGSNNFDVV |
| | (SEQ ID NO: 805) | (SEQ ID NO: 806) | (SEQ ID NO: 807) |
| Ab_15 | SGTIASNY | EDN | QSYDTSNHYV |
| | (SEQ ID NO: 808) | (SEQ ID NO: 809) | (SEQ ID NO: 810) |
| Ab_16 | SSNIGAGYD | GTN | QSYDNSLTDPYV |
| | (SEQ ID NO: 811) | (SEQ ID NO: 812) | (SEQ ID NO: 813) |
| Ab_17 | SSDVGGSKY | DVT | AAWDDSLNGVV |
| | (SEQ ID NO: 814) | (SEQ ID NO: 815) | (SEQ ID NO: 816) |
| Ab_18 | SGSIANNF | EDN | QSYDSSNLV |
| | (SEQ ID NO: 817) | (SEQ ID NO: 818) | (SEQ ID NO: 819) |
| Ab_19 | SANIGSNA | GNT | AAWDDSLNGYV |
| | (SEQ ID NO: 820) | (SEQ ID NO: 821) | (SEQ ID NO: 822) |
| Ab_20 | SGSIASNY | EDN | QSYDSSNHWV |
| | (SEQ ID NO: 823) | (SEQ ID NO: 824) | (SEQ ID NO: 825) |
| Ab_21 | SSDVGGYDY | DVS | SSYTSSSPVV |
| | (SEQ ID NO: 826) | (SEQ ID NO: 827) | (SEQ ID NO: 828) |
| Ab_22 | TGSIAGNY | DDN | QSYDSGNRGV |
| | (SEQ ID NO: 829) | (SEQ ID NO: 830) | (SEQ ID NO: 831) |
| Ab_23 | SRDIGAYGY | EVR | SSYTSSSTLDVV |
| | (SEQ ID NO: 832) | (SEQ ID NO: 833) | (SEQ ID NO: 834) |
| Ab_24 | SSNIGRNA | SNN | SAWDTSLSTWV |
| | (SEQ ID NO: 835) | (SEQ ID NO: 836) | (SEQ ID NO: 837) |
| Ab_25 | SGSIASNY | EDN | QSYDSSNHWV |
| | (SEQ ID NO: 838) | (SEQ ID NO: 839) | (SEQ ID NO: 840) |
| Ab_26 | NIRTKG | YAS | QVWDSSSDLVV |
| | (SEQ ID NO: 841) | (SEQ ID NO: 842) | (SEQ ID NO: 843) |
| Ab_27 | SSDVGTYNR | EVS | SSYTRTFTYV |
| | (SEQ ID NO: 844) | (SEQ ID NO: 845) | (SEQ ID NO: 846) |
| Ab_28 | GGSIASNY | EDN | QSYDSSNPVV |
| | (SEQ ID NO: 847) | (SEQ ID NO: 848) | (SEQ ID NO: 849) |
| Ab_38 | QSVSSN | DVS | QQRGAWPLT |
| | (SEQ ID NO: 986) | (SEQ ID NO: 987) | (SEQ ID NO: 988) |
| wcS2-T4-E7 | SGDIATRH | ESN | QSYDSTNPWV |
| | (SEQ ID NO: ) | (SEQ ID NO: ) | (SEQ ID NO: ) |
| wcS2-E1-A9 | SSDVGAYNY | EVS | SSYAGTRKYYV |
| | (SEQ ID NO: ) | (SEQ ID NO: ) | (SEQ ID NO: ) |
| wcS2-T4-H8 | QIVTNNN | GAS | QQYYYWPLS |
| | (SEQ ID NO: ) | (SEQ ID NO: ) | (SEQ ID NO: ) |
| wcS2-T3-F5 | SSDVGGYKY | DVS | SSFSQTNSYV |
| | (SEQ ID NO: ) | (SEQ ID NO: ) | (SEQ ID NO: ) |
| wcS2-E2-C1 | SSDVGRYKY | EVN | SSYAGSNNPYV |
| | (SEQ ID NO: ) | (SEQ ID NO: ) | (SEQ ID NO: ) |
| wcS2-T4-C9 | QNVPSNS | GAS | QLYDRSSQLA |
| | (SEQ ID NO: ) | (SEQ ID NO: ) | (SEQ ID NO: ) |
| wcS2-T3-B9 | SLGTYY | GKN | NSRNNSGYHE |
| | (SEQ ID NO: ) | (SEQ ID NO: ) | (SEQ ID NO: ) |
| wcS2-E3-C8 | QSNIGSNT | VNN | SSYAGSNDYV |
| | (SEQ ID NO: ) | (SEQ ID NO: ) | (SEQ ID NO: ) |
| wcS2-T4-F8 | SGSIASNY | GDN | QSFDGSYHWV |
| | (SEQ ID NO: ) | (SEQ ID NO: ) | (SEQ ID NO: ) |
| wcS2-T3-F1 | SSNIGSNT | SNN | ATWDDGLSGRV |
| | (SEQ ID NO: ) | (SEQ ID NO: ) | (SEQ ID NO: ) |
| wcS2-E2-B1 | SLRSYY | GKN | NSRDSSGNHLRV |
| | (SEQ ID NO: ) | (SEQ ID NO: ) | (SEQ ID NO: ) |
| wcS2-T2-G3 | SNNVGNQG | RNN | SAWDNSLGAWV |
| | (SEQ ID NO: ) | (SEQ ID NO: ) | (SEQ ID NO: ) |
| wcS2-T1-A6 | QSNIGSNT | VNN | SSYAGSNDYV |
| | (SEQ ID NO: ) | (SEQ ID NO: ) | (SEQ ID NO: ) |
| wcS2-T4-D4 | SGDIATRH | ESN | LTYDITNPWV |
| | (SEQ ID NO: ) | (SEQ ID NO: ) | (SEQ ID NO: ) |
| wcS2-T2-D10 | SGDIATKH | DND | SYDDSTNPCV |
| | (SEQ ID NO: ) | (SEQ ID NO: ) | (SEQ ID NO: ) |
| wcS2-T1-G9 | QSISSSY | DAS | QQRGNWPLT |
| | (SEQ ID NO: ) | (SEQ ID NO: ) | (SEQ ID NO: ) |
| wcS2-E3-H7 | QNVPSNS | GAS | QLYDRSSQLA |
| | (SEQ ID NO: ) | (SEQ ID NO: ) | (SEQ ID NO: ) |
| wcS2-T2-C11 | SLTIFF | KDT | QSADTSGTLKV |
| | (SEQ ID NO: ) | (SEQ ID NO: ) | (SEQ ID NO: ) |
| SARS2-R3- | SSDLGGHNF | GVN | SSYEATHIYV |
| G2-P1- | | | |
| H9_PelB- | | | |
| F_2020-06- | | | |
| 19_D08 | | | |
| SARS2-R3- | SSDVGSYNL | EVS | SSFSSGSIPYV |
| G3-P1- | | | |
| F8_PelB- | | | |
| F_2020-06- | | | |
| 19_F11 | | | |
| SARS2-R3- | QSVSTN | GAS | QHYANWPRT |
| G2-P1- | | | |
| B4_PelB- | | | |
| F_2020-06- | | | |
| 19_B02 | | | |
| SARS2-R3- | SSNIGSNT | SNN | ATWDDSLNGPV |
| G3-P1- | | | |
| B2_PelB- | | | |
| F_2020-06- | | | |
| 19_A08 | | | |
| SARS2-R3- | NIGSKS | DDT | QVWDTSSDHPYV |
| G3-P1- | | | |
| B3_PelB- | | | |
| F_2020-06- | | | |
| 19_B08 | | | |
| SARS2-R3- | SLRSYY | GGN | SSRATSAFYV |
| G1-P3- | | | |
| E8_PelB- | | | |
| F_2020-06- | | | |
| 19_H04 | | | |
| SARS2-R3- | SSNIGAGYD | GNS | QSYDSSLSGWV |
| G1-P2- | | | |
| D7_PelB- | | | |
| F_2020-06- | | | |
| 19_H09 | | | |
| SARS2-R3- | ALPNRY | KDS | QSVNTIGTYV |
| G1-P1- | | | |
| C6_PelB- | | | |
| F_2020-06- | | | |
| 19_H02 | | | |
| SARS2-R3-T- | SSDLVGHKF | EIN | TCYDEDHIYV |
| P2-E12_PelB- | | | |
| F_2020-06- | | | |
| 19_D07 | | | |
| SARS2-R3- | ENVNNW | KAS | QQYKSYS |
| G2-P1- | | | |
| D12_PelB- | | | |
| F_2020-06- | | | |
| 19_H04 | | | |
| SARS2-R3- | SGSIASNY | AHN | HSYDTNNPVV |
| G1-P2- | | | |
| E8_PelB- | | | |
| F_2020-06- | | | |
| 19_E10 | | | |
| SARS2-R3- | QSVRSN | GVS | QHYGSSPLYT |
| G1-P1- | | | |
| A10_PelB- | | | |
| F_2020-06- | | | |
| 19_H01 | | | |
| SARS2-R3- | SSDVGSYNR | EVS | SSYTSSSPYV |
| G1-P3- | | | |
| B5_PelB- | | | |
| F_2020-06- | | | |
| 19_D02 | | | |
| SARS2-R3- | IGDIARNY | EDD | QSYDTTIV |
| G3-P1- | | | |
| A2_PelB- | | | |
| F_2020-06- | | | |
| 19_B07 | | | |
| SARS2-R3- | SSNIGAGYH | GNQ | SAWDDSLGGEV |
| G3-P1- | | | |
| D1_PelB- | | | |
| F_2020-06- | | | |
| 19_G09 | | | |
| SARS2-R3- | SNNVGHEG | KND | SAWDSSLGSWV |
| G1-P2- | | | |
| D4_PelB- | | | |
| F_2020-06- | | | |
| 19_G09 | | | |
| SARS2-R3- | RSNIGSNT | SNN | QSYDSSVV |
| G1-P1- | | | |
| B6_PelB- | | | |
| F_2020-06- | | | |
| 19_E02 | | | |
| SARS2-R3- | NLGEIY | QDK | QAWDSSTGV |
| G3-P1- | | | |
| G8_PelB- | | | |
| F_2020-06- | | | |
| 25_A05 | | | |
| SARS2-R3- | ALPDKY | KDS | QSADSSGTWV |
| G1-P1- | | | |
| F11_PelB- | | | |
| F_2020-06- | | | |
| 25_B05 | | | |
| SARS2-R3- | SSDVGGYNH | DVS | TSYAGSNSLV |
| T1-P3- | | | |
| F4_PelB- | | | |
| F_2020-06- | | | |
| 20_C03 | | | |
| SARS2-R3- | SGDVGGYNS | EVT | SSYAGSNNYV |
| G1-P2- | | | |
| B8_PelB- | | | |
| F_2020-06- | | | |
| 25_C06 | | | |
| SARS2-R3- | SGSIASNY | EDN | QSYDSPWV |
| G1-P2- | | | |
| D3_PelB- | | | |
| F_2020-06- | | | |
| 25_C12 | | | |
| SARS2-R3- | SSNIGSNT | SNN | AVWDDSLNGLV |
| G1-P4- | | | |
| C5_PelB- | | | |
| F_2020-06- | | | |
| 25_C07 | | | |
| SARS2-R3- | SNNVGHEG | RNK | SAWDGRLNGYL |
| G1-P2- | | | |
| D6_PelB- | | | |
| F_2020-06- | | | |
| 25_D02 | | | |
| SARS2-R3- | KLEDKF | EDA | QAWAITTEV |
| G1-P1- | | | |
| B7_PelB- | | | |
| F_2020-06- | | | |
| 25_A03 | | | |
| 95_PelB- | QSVSSTY | GAS | QHSYRTPYT |
| F_2020-06- | | | |
| 25_G11 | | | |
| 73_PelB- | QSVRDGY | GAS | QQRSNWPPT |
| F_2020-06- | | | |
| 25_F09 | | | |
| SARS2-R3- | SDDVGGFAH | DVS | SSYAGRNGVI |
| G3-P1- | | | |
| G9_PelB- | | | |
| F_2020-06- | | | |
| 25_B05 | | | |
| SARS2-R3- | SNNVGDQG | RNN | SAWDSSLSAQV |
| G1-P3- | | | |
| G7_PelB- | | | |
| F_2020-06- | | | |
| 25_H01 | | | |
| SARS2-R3- | SGSIASNY | EDN | QSYDNNNHVV |
| G1-P4- | | | |
| A2_PelB- | | | |
| F_2020-06- | | | |
| 25_A06 | | | |
| SARS2-R3- | SSNIGAGYD | GNS | QSYDSSLSGWV |
| G3-P1- | | | |
| G1_PelB- | | | |
| F_2020-06- | | | |
| 25_F04 | | | |
| SARS2-R3- | SSDLGGHNF | DVY | SSYAGSNPYV |
| G3-P1- | | | |
| F1_PelB- | | | |
| F_2020-06- | | | |
| 25_C04 | | | |
| SARS2-R3- | RSNIGINS | RNN | AAWDGSLNGPL |
| G1-P3- | | | |
| H10_PelB- | | | |
| F_2020-06- | | | |
| 25_H10 | | | |
| 85_PelB- | SSNIGNYG | SNN | AAWDDSLKGV |
| F_2020-06- | | | |
| 25_G10 | | | |
| SARS2-R3- | TSNIGRNK | LNN | AAWDDSLTGYV |
| G1-P4- | | | |
| A3_PelB- | | | |
| F_2020-06- | | | |
| 25_B06 | | | |
| SARS2-R3- | SLSRYY | GKD | NSRDSSGNL |
| G1-P3- | | | |
| H6_PelB- | | | |
| F_2020-06- | | | |
| 25_H08 | | | |
| SARS2-R3- | SSDVGGYNY | DVN | SSYTSSSTYV |
| G3-P1- | | | |
| G5_PelB- | | | |
| F_2020-06- | | | |
| 25_H04 | | | |
| SARS2-R3- | GLPKHY | KDT | QSGDTSGPVV |
| G1-P4- | | | |
| C10_PelB- | | | |
| F_2020-06- | | | |
| 25_F07 | | | |
| SARS2-R3- | SSNIGRGYN | GNS | SAWDDSLNNVV |
| G1-P1- | | | |
| F10_PelB- | | | |
| F_2020-06- | | | |
| 25_B04 | | | |
| SARS2-R3- | NIGHKG | DDS | QVWESYGDHVV |
| T1-P4- | | | |
| H4_PelB- | | | |
| F_2020-06- | | | |
| 20_H04 | | | |
| SARS2-R3- | QSLLHSNGYNY | FGS | MQVLQTPPIT |
| G2-P1- | | | |
| E8_PelB- | | | |
| F_2020-06- | | | |
| 25_A02 | | | |
| SARS2-R3- | TSNIGAGYD | DNN | QSYDSGLDGSV |
| G1-P1- | | | |
| C3_PelB- | | | |
| F_2020-06- | | | |
| 25_A07 | | | |
| SARS2-R3- | SSDVGGYNH | DVS | TSYAGSNSLV |
| G3-P1- | | | |
| G2_PelB- | | | |
| F_2020-06- | | | |
| 25_G04 | | | |
| SARS2-R3- | SGSIASNY | EDD | QSYDNNNAVI |
| G1-P4- | | | |
| DI_PelB- | | | |
| F_2020-06- | | | |
| 25_A08 | | | |

The amino acid sequences of the heavy and light chain framework regions of the COVID-19 antibodies are shown in Table 64A-B below:

TABLE 64A
Heavy chain (V_{H}) framework regions (FRs) of the COVID-19 antibodies.

| Seq ID | FR1-IMGT | FR2-IMGT | FR3-IMGT | FR4-IMGT |
|---|---|---|---|---|
| S1-R3- | EVQLVESGPGV | WNWIRQPPGKPL | NKNPSLK.SRVTIS | WSRGTL |
| T1-H7 | VSPSATLFLTCS | EWIGF | SDTSKNEFSLTLT | VTVSS |
| (Ab12) | VS | (SEQ ID NO: 363) | SVTAADTAVYYC | (SEQ ID |
| | (SEQ ID NO: 362) | | (SEQ ID NO: 364) | NO: 365) |
| RBD-R3- | EVQLVESGPGL | WSWIRQPAGKG | NYNPSLKSRVTM | WGQGTL |
| E1-G7 | VKPSETLSLTCT | LEWIGR | SVDTSKNQFSLKL | VTVSS |
| (Ab2-10) | VS | (SEQ ID NO: 367) | SSVTAADTAVYY | (SEQ ID |
| | (SEQ ID NO: 366) | | C | NO: 369) |
| | | | (SEQ ID NO: 368) | |
| S1-RBD- | EVQLVESGPGL | WNWIR*PPGKPL | NKNPSLKSRVSIS | WSRGTL |
| R3-E1- | VKPSATLFLTCS | EWIGF | SDPSKNEFSLTLT | VTVSS |
| D8 | VS | (SEQ ID NO: 371) | SVTAADTAVYYC | (SEQ ID |
| (Ab_3) | (SEQ ID NO: 370) | | (SEQ ID NO: 372) | NO: 373) |
| S1-RBD- | QVQLQQSGPGL | WNWIRQSPSRGL | DYAVSVKSRITIN | WGQGTT |
| R3-T1- | VKPSQTLSLTCA | EWLGR | PDTSKNQFSLQLS | VTVSS |
| C7 | IS | (SEQ ID NO: 375) | SVTPEDTAVYYC | (SEQ ID |
| | (SEQ ID NO: 374) | | (SEQ ID NO: 376) | NO: 377) |
| RBD-R3- | QVQLVQSGGGV | MHWVRQAPGK | YYADSVKGRFTIS | WGQGTT |
| T1-F4 | VQPGRSLRLSC | GLEWLAV | RDKSKNTLYLQIN | VTVSS |
| (Ab2-2) | AAS | (SEQ ID NO: 379) | SLRAEDTAVYYC | (SEQ ID |
| | (SEQ ID NO: 378) | | (SEQ ID NO: 380) | NO: 381) |
| RBD-R3- | QVQLVQSGG.G | IHWVRQAPGKG | YYGDSVKGRFTIS | WGQGTT |
| E1-A5 | VVQPGKSLRLS | LEWVGV | RDNAKNSLYLQM | VTVSS |
| | CTAS | (SEQ ID NO: 383) | NSLRVEDTAVYY | (SEQ ID |
| | (SEQ ID NO: 382) | | C | NO: 385) |
| | | | (SEQ ID NO: 384) | |
| S1-RBD- | QVTLKESGPTR | VGWIRQPPGKAL | RYSPSLKSRLTIT | WGQGTT |
| R3-T1- | VKPTQTLTLTCT | EWLAL | KDTSKNQVVLTM | VTVSS |
| F5 | FS | (SEQ ID NO: 387) | TNMDPVDTATYY | (SEQ ID |
| (Ab2-7) | (SEQ ID NO: 386) | | C | NO: 389) |
| | | | (SEQ ID NO: 388) | |
| S1-R3- | QVQLVQSGGGV | MHWVRQAPGK | NYADFVEGRFTIS | WGQGTL |
| T1-A12 | VQPGRSLRLSC | GLEWVAV | RDNSKNTLYLQM | VTVSS |
| (Ab_5) | AAS | (SEQ ID NO: 391) | NSLRPEDTGVYY | (SEQ ID |
| | (SEQ ID NO: 390) | | C | NO: 393) |
| | | | (SEQ ID NO: 392) | |
| S1-R3- | QVQLVQSGGGL | MHWVRQAPGK | PYADSVKGRFTIS | WGQGTL |
| T1-A6 | VQPGRSLRLSC | GLEWVAA | RDNAKNSLYLQM | VTVSS |
| (Ab_4) | AAS | (SEQ ID NO: 395) | NSLRDEDTAVYY | (SEQ ID |
| | (SEQ ID NO: 394) | | C | NO: 397) |
| | | | (SEQ ID NO: 396) | |
| S1-RBD- | QVQLVQSGAEV | IHWVRQAPGQG | NFAQRFQGRVTM | WGQGTL |
| R3-T1- | KKPGASVKFSC | LEWMGW | TTDTSVSTAYMD | VTVSS |
| A5 | KAS | (SEQ ID NO: 399) | LRRLRSDDTAVY | (SEQ ID |
| | (SEQ ID NO: 398) | | YC | NO: 401) |
| | | | (SEQ ID NO: 400) | |
| S1-RBD- | QVHLVQSGGGV | MHWVRQAPGK | YYAESVKGRFTIS | WGQGTM |
| R3-T1- | VQPGRSLRLSC | GLEWVAL | RDNSKNTLYLQM | VTVSS |
| B3 | AAS | (SEQ ID NO: 403) | NSLRAEDTAVYY | (SEQ ID |
| (Ab_1) | (SEQ ID NO: 402) | | C | NO: 405) |
| | | | (SEQ ID NO: 404) | |
| S1-RBD- | QVQLVQSGTEV | LHWVRQAPGQG | NYAQKFQGRVTL | WGQGTL |
| R3-T1- | KKPGASVKVSC | LEWMGW | TRDTSISTAYMEL | VTVSS |
| E7 | KAS | (SEQ ID NO: 407) | SGLRSDDTAVYY | (SEQ ID |
| | (SEQ ID NO: 406) | | C | NO: 409) |
| | | | (SEQ ID NO: 408) | |
| S1-RBD- | QVTLKESGPTL | VSWIRQPPGKAL | FYSTALKTRLTIS | WGRGTL |
| R3-T1- | VKPTQTLTLTCT | EWLAR | KDTSKNQVVFTM | VTVSS |
| F9 | LS | (SEQ ID NO: 411) | TSMDPVDTATYY | (SEQ ID |
| | (SEQ ID NO: 410) | | C | NO: 413) |
| | | | (SEQ ID NO: 412) | |
| S1-R3- | QVQLVQSGGGV | FHWVRQAPGKG | LYADSVKGRFTIS | WGQGTT |
| T1-C2 | VQPGRSLRLSC | LQWVAV | RDDSQNMLYLEM | VTVSS |
| (Ab_6) | AAS | (SEQ ID NO: 415) | HSLRLEDTAVYY | (SEQ ID |
| | (SEQ ID NO: 414) | | C | NO: 417) |
| | | | (SEQ ID NO: 416) | |
| RBD-R3- | QVQLVQSGAEV | IGWVRQMPGKG | RYSPSFQGQVTIS | WGQGTL |
| E1-B3 | KKPGESLKISCK | LEWMGI | ADKSISTAYLQW | VTVSS |
| | GS | (SEQ ID NO: 419) | SSLKASDTAMYY | (SEQ ID |
| | (SEQ ID NO: 418) | | C | NO: 421) |
| | | | (SEQ ID NO: 420) | |
| RBD-R3- | QVQL.QSGPGL | WSWIRQSPSRGL | DFAQSVRSRIVIN | WGQGTL |
| T1-B5 | VKASQTLSLTC | EWLAR | PDTSKNHVYLQL | VTVSS |
| | VIS | (SEQ ID NO: 423) | RSVTPEDTAVYY | (SEQ ID |
| | (SEQ ID NO: 422) | | C | NO: 425) |
| | | | (SEQ ID NO: 424) | |
| RBD-R3- | EVQLVQSGAEV | ISWVRQAPGQGL | NYAQKLQGRVT | WGKGTL |
| T1-H3 | KKPGSSVKVSC | EWMGW | MTTDTSTSTAYM | VTVSS |
| (Ab_2) | KTS | (SEQ ID NO: 427) | ELRSLRSDDTAV | (SEQ ID |
| | (SEQ ID NO: 426) | | YYC | NO: 429) |
| | | | (SEQ ID NO: 428) | |
| S1-R3- | QVQLVQSGAEV | ISWLRQAPGQGL | NYAQKFQGRVT | WGQGTL |
| T1-C4 | KKPGSSVKVSC | EWMGW | MTRDTSISTAYM | VTVSS |
| (Ab_8) | KAS | (SEQ ID NO: 431) | ELSRLRSDDTAV | (SEQ ID |
| | (SEQ ID NO: 430) | | YYC | NO: 433) |
| | | | (SEQ ID NO: 432) | |
| S1-RBD- | QVQLVQSGAEV | ISWVRQAPGQGL | NYAQKFQDRVAI | WGQGTL |
| R3-T1- | KKPGSSVKVSC | EWMGG | TADKSTSTAYME | VTVSS |
| B12 | KAS | (SEQ ID NO: 435) | LSSLRSEDTAVYY | (SEQ ID |
| | (SEQ ID NO: 434) | | C | NO: 437) |
| | | | (SEQ ID NO: 436) | |
| S1-RBD- | QVQLVQSGGGL | MSWVRQAPGKG | YYVDSVKGRFTIS | WGQGTT |
| R3-T1- | VQPGGSLRLSC | LEWVAN | RDNAKNSLYLQM | VTVSS |
| G5 | AAS | (SEQ ID NO: 439) | NSLRAEDTAVYY | (SEQ ID |
| | (SEQ ID NO: 438) | | C | NO: 441) |
| | | | (SEQ ID NO: 440) | |
| S1-RBD- | QVQLVQSGGGL | MHWVRQVPGK | GYADSVKGRFTIS | WGQGTM |
| R3-T1- | VQPGRSLRLSC | GLEWVSG | RDNAKNSLYLQM | VTVSS |
| E2 (Ab7) | AAS | (SEQ ID NO: 443) | NSLRAEDTALYY | (SEQ ID |
| | (SEQ ID NO: 442) | | C | NO: 445) |
| | | | (SEQ ID NO: 444) | |
| RBD-R3- | QVQLVQSGGGL | MHWVRQVPGK | GYADSVKGRFTIS | WGQGTT |
| T1-F7 | VQPGRSLRLSC | GLEWVSG | RDNVKNSLYLQM | VTVSS |
| | AAS | (SEQ ID NO: 447) | TSLRAEDTAVYF | (SEQ ID |
| | (SEQ ID NO: 446) | | C | NO: 449) |
| | | | (SEQ ID NO: 448) | |
| S1-RBD- | EVQLVQSGAEV | IGWVRQMPGKG | KYSPSFQGQVTIS | WGQGTL |
| R3-T1- | KKPGESLRISCK | LEWMGS | ADRSISTAYLQWS | VTVSS |
| G1 | AS | (SEQ ID NO: 451) | GLKASDTAMYYC | (SEQ ID |
| | (SEQ ID NO: 450) | | (SEQ ID NO: 452) | NO: 453) |
| S1-RBD- | EVQLVQSGAEV | ISWVRQAPGQGL | KYAQKLQGRVT | WGQGTL |
| R3-T1- | KKPGASVKVSC | EWMGW | MTTDTSTSTAYM | VTVSS |
| C2 | KAS | (SEQ ID NO: 455) | ELRSLRSDDTAV | (SEQ ID |
| | (SEQ ID NO: 454) | | YYC | NO: 457) |
| | | | (SEQ ID NO: 456) | |
| S1-RBD- | EVQLVQSGAEV | ISWVRQAPGQGL | RYAQKFQGRVTL | WGQGTM |
| R3-T1- | EKPGSSVKVSC | EWMGW | TIDTSSSTAYMEL | VTVSS |
| H8 | KAS | (SEQ ID NO: 459) | SSLRSEDTAVYYC | (SEQ ID |
| | (SEQ ID NO: 458) | | (SEQ ID NO: 460) | NO: 461) |
| S1-RBD- | QVQLVQSGAEV | ISWVRQAPGQGL | NYAQKLQGRVT | WGQGTL |
| R3-E1- | KKPGASVKVSC | EWMGW | MTTDTSTSTAYM | VTVSS |
| E8 | KAS | (SEQ ID NO: 463) | EVRSLRSDDTAV | (SEQ ID |
| | (SEQ ID NO: 462) | | YYC | NO: 465) |
| | | | (SEQ ID NO: 464) | |
| RBD-R3- | QVTLKESGPTL | VGWIRQPPGKAL | RYSPSLRSRLTITR | WGQGTL |
| T1-H2 | VKPTQTLTLTCT | EWLAL | DTSKNQVVLTVT | VTVSS |
| | FS | (SEQ ID NO: 467) | DMDPADTGTYYC | (SEQ ID |
| | (SEQ ID NO: 466) | | (SEQ ID NO: 468) | NO: 469) |
| S1-RBD- | QVTLKESGPAL | VSWIRQSPGKAL | YYTTSLKTRLTIT | WGQGTL |
| R3-T1- | VKSTQTLTLTCT | EWLAL | KDTSKNQVVLTM | VTVSS |
| B7 | IS | (SEQ ID NO: 471) | TSMDPLDTATYY | (SEQ ID |
| | (SEQ ID NO: 470) | | C | NO: 473) |
| | | | (SEQ ID NO: 472) | |
| S1-RBD- | QVQLVQSGGGL | MTWFRQAPGKG | KYADSVKGRFTIS | WGQGTL |
| R3-E1- | VKPGGSLRLSC | LEWISY | RDNAKNSLYLQM | VTVSS |
| E5 | AAS | (SEQ ID NO: 475) | DSLRAEDTAVYY | (SEQ ID |
| | (SEQ ID NO: 474) | | C | NO: 477) |
| | | | (SEQ ID NO: 476) | |
| S1-R3- | QVQLVQSGGGV | VHWVRQAPGKG | YYADSVKGRFTL | WGHGTL |
| T1-H6 | VQPGKSLRLSC | LEWVAV | SRDNSKNTVYLQ | VTVSS |
| | AAS | (SEQ ID NO: 479) | LSSLRREDTAVY | (SEQ ID |
| | (SEQ ID NO: 478) | | YC | NO: 481) |
| | | | (SEQ ID NO: 480) | |
| S1-RBD- | QVQLVQSGGGV | MHWVRQAPGK | YYADSVKGRFTIS | WGQGTM |
| R3-E1- | VQPGRSLRLSC | GLEYVSA | RDNGKNSLYLQM | VTVSS |
| C6 | AAS | (SEQ ID NO: 483) | SSLRAEDTAVYY | (SEQ ID |
| | (SEQ ID NO: 482) | | C | NO: 485) |
| | | | (SEQ ID NO: 484) | |
| S1-RBD- | QVQLVQSGGGL | MSWVRQAPGKG | YYVDSVKGRFTIS | WGQGTT |
| R3-E1- | VQPGGSLRLSC | LEWVAN | RDNAKNSLYLQM | VTVSS |
| F2 | AAS | (SEQ ID NO: 487) | NGLRAEDTAVYY | (SEQ ID |
| | (SEQ ID NO: 486) | | C | NO: 489) |
| | | | (SEQ ID NO: 488) | |
| S1-RBD- | QVQLVQSGGGL | MHWVRQVPGK | GYADSVKGRFTIS | WGQGTM |
| R3-T1- | VQPGRSLRLSC | GLEWVSG | RDNAKNSLYLQM | VTVSS |
| C3 | AAS | (SEQ ID NO: 491) | NSLRAEDTALYY | (SEQ ID |
| | (SEQ ID NO: 490) | | C | NO: 493) |
| | | | (SEQ ID NO: 492) | |
| S1-RBD- | QVQLVQSGGGL | MHWVRQAPGK | GYVDSVKGRFTIS | WGLGTL |
| R3-T1- | VQPGGSLRLSC | GLEWVSG | RDNRNNKVYLQ | VTVSS |
| G12 | ATS | (SEQ ID NO: 495) | MNNLRAEDTAVY | (SEQ ID |
| | (SEQ ID NO: 494) | | YC | NO: 497) |
| | | | (SEQ ID NO: 496) | |
| S1-RBD- | EVQLVESGPGL | WSWVRQPPGKG | NYNPSLKSRVTIS | WGQGTM |
| R3-E1- | VKPSGTLSLTCA | LEWIGE | VDTSKNQFSLKLS | VTVSS |
| F1 | VS | (SEQ ID NO: 499) | SVTAADTAVYYC | (SEQ ID |
| | (SEQ ID NO: 498) | | (SEQ ID NO: 500) | NO: 501) |
| S1-RBD- | QVTLKESGPGL | WSWIRQSPGKGL | IYNPSLKSRLTMSI | WGQGTL |
| R3-E1- | VNPSETLSLTCT | EWIGY | DTSKNQLSLNLRS | VTVSS |
| H8 | VS | (SEQ ID NO: 503) | LTAADTAVYYC | (SEQ ID |
| | (SEQ ID NO: 502) | | (SEQ ID NO: 504) | NO: 505) |
| S1-RBD- | EVQLVESGPGL | WNWIRQTPGKP | NKNPPLLSRVSISS | WSGGTL |
| R3-T1- | MKPSETLFLSCS | LEWMGI | DPSNNEFSLTLTS | ATVSS |
| F1 | VS | (SEQ ID NO: 507) | VTAADTAVYYC | (SEQ ID |
| | (SEQ ID NO: 506) | | (SEQ ID NO: 508) | NO: 509) |
| S1-R3- | EVQLVESGPGL | WNWIRQAPGKP | NKNPSLKSRVTIS | WSRGTL |
| T1-B10 | VSPSATLFLTCS | LEWMGV | SETSDNKFSLTLT | GTVSS |
| | VS | (SEQ ID NO: 511) | SVTAEDTAVYYC | (SEQ ID |
| | (SEQ ID NO: 510) | | (SEQ ID NO: 512) | NO: 513) |
| RBD-R3- | EVQLVESGPGV | WNWIRQPPGKPL | NKNPSLKSRVTIS | WSRGTL |
| E1-D12 | VSPSATLFLTCS | EWIGV | SKTSDNKFSLTLT | GTVSS |
| | VS | (SEQ ID NO: 515) | SVTAEDTAVYYC | (SEQ ID |
| | (SEQ ID NO: 514) | | (SEQ ID NO: 516) | NO: 517) |
| RBD-R3- | EVQLVESGPGV | WNWIRQAPGKA | NNNPSLKSRVTIS | WSRGTL |
| T1-C5 | VSPSATLFLTCS | LEWIGF | SDTSKNEFSLTLT | VTVSS |
| | VS | (SEQ ID NO: 519) | SVTAADTAVYYC | (SEQ ID |
| | (SEQ ID NO: 518) | | (SEQ ID NO: 520) | NO: 521) |
| S1-RBD- | QVQLVQSGAEV | IGWVRQMPGKG | RYSPSFQGQVTIS | WGQGTM |
| R3-T1- | KKPGESLKISCK | LEWMGI | ADKSISTAYLQW | VTVSS |
| B4 | GS | (SEQ ID NO: 523) | SSLKASDTAMYY | (SEQ ID |
| | (SEQ ID NO: 522) | | C | NO: 525) |
| | | | (SEQ ID NO: 524) | |
| S1-RBD- | QVQLVQSGAEV | IAWVRQMPGKG | RYSPSLQGQVTIS | WGQGTT |
| R3-E1- | KKPGNSLKISCK | LEWLGS | VDKSLSTAYLQW | VTVSS |
| E7 | GS | (SEQ ID NO: 527) | RSLKASDTAMYY | (SEQ ID |
| | (SEQ ID NO: 526) | | C | NO: 529) |
| | | | (SEQ ID NO: 528) | |
| S1-RBD- | EVQLVQSGAEV | IGWVRQMPGKG | RYSPSFQGQVTIS | WGQGTL |
| R3-T1- | KKPGESLKISCK | LEWMGI | ADKSISTAYLQW | VTVSS |
| C8 | GS | (SEQ ID NO: 531) | SSLKASDTAMYY | (SEQ ID |
| | (SEQ ID NO: 530) | | C | NO: 533) |
| | | | (SEQ ID NO: 532) | |
| S1-RBD- | QVQLVQSGVEV | IGWVRQMPGKG | RYSPSFQGQVTIS | WGQGTL |
| R3-T1- | KKPGESLKISCK | LEWMGI | ADKSISTAYLQW | VTVSS |
| D7 | GS | (SEQ ID NO: 535) | SSLKASDTAMYY | (SEQ ID |
| | (SEQ ID NO: 534) | | C | NO: 537) |
| | | | (SEQ ID NO: 536) | |
| RBD-R3- | QVQLQQSGPGL | WNWIRQSPSRGL | DYAVSVKSRVTI | WGQGTL |
| E1-F5 | VKPSQTLSLTCA | EWLGR | NADTSKNQLSLQ | VTVSS |
| | IF | (SEQ ID NO: 539) | LNSVTPEDTAVY | (SEQ ID |
| | (SEQ ID NO: 538) | | YC | NO: 541) |
| | | | (SEQ ID NO: 540) | |
| Ab_13 | QVTLKESGPKL | VGWIRQPPGKAL | RYSPSLKSRLTIA | WGQGTM |
| | VKPTQTLTLTCT | EWLAL | KDTSKYQVVLTM | VTVSA |
| | FS | (SEQ ID NO: 851) | TNMDPVDTATYY | (SEQ ID |
| | (SEQ ID NO: 850) | | C | NO: 853) |
| | | | (SEQ ID NO: 852) | |
| Ab_14 | QVQLQQSGPGL | WNWIRQSPARG | DYAISMKSRITINP | WGQGTL |
| | VKPSQTLSLTCA | LEWLGR | DTSKNQFSLQLES | VTVSS |
| | IS | (SEQ ID NO: 855) | VTPEDTAVYYC | (SEQ ID |
| | (SEQ ID NO: 854) | | (SEQ ID NO: 856) | NO: 857) |
| Ab_15 | QVQLVQSGGGL | MSWFRQAPGKG | GYAASVKGRFTIS | WGKGTT |
| | VKPGRSLRLSCT | LEWVGF | RDDSKSIAYLQM | VTVSS |
| | AS | (SEQ ID NO: 859) | NSLKTEDTAVYY | (SEQ ID |
| | (SEQ ID NO: 858) | | C | NO: 861) |
| | | | (SEQ ID NO: 860) | |
| Ab_16 | QVQLVQSGGGV | MHWVRQAPGK | DYADSVKGRFTV | WGQGTL |
| | VQPGRSLRLSC | GLEWVAL | SRDNSKNTLYLQ | VTVSS |
| | AAS | (SEQ ID NO: 863) | MNSLRPEDTAVY | (SEQ ID |
| | (SEQ ID NO: 862) | | FC | NO: 865) |
| | | | (SEQ ID NO: 864) | |
| Ab_17 | QVQLVQSGGGV | MHWVRQVPGK | GYADSVKGRFTIS | WGQGTT |
| | VQPGRSLRLSC | GLEWVSG | RDNVKNSLYLQM | VTVSS |
| | AAS | (SEQ ID NO: 867) | NSLRTEDSALYY | (SEQ ID |
| | (SEQ ID NO: 866) | | C | NO: 869) |
| | | | (SEQ ID NO: 868) | |
| Ab_18 | EVQLVQSGGGV | MHWVRQAPGK | YYADSVEGRFTIS | WGQGTL |
| | VQPGRSLRVSC | GLEWVAF | RDNSRNTVSLEM | VTVSS |
| | AAS | (SEQ ID NO: 871) | NSLRGEDTAVYY | (SEQ ID |
| | (SEQ ID NO: 870) | | C | NO: 873) |
| | | | (SEQ ID NO: 872) | |
| Ab_19 | EVQLVESGAEV | IGWVRQMPGKG | RYSPSFQGQVTIS | WGQGTM |
| | KKPGESLKISCK | LEWMGI | ADKSISTAYLQW | VTVSS |
| | GS | (SEQ ID NO: 875) | SSLKASDTAMYY | (SEQ ID |
| | (SEQ ID NO: 874) | | C | NO: 877) |
| | | | (SEQ ID NO: 876) | |
| Ab_20 | QVQLVQSGGGS | MHWVRQAPGK | YYVDSVKGRFTIS | WGQGTL |
| | VKPGGSLRLSC | GLEWVAN | RDNAKNSLYLQM | VTVSS |
| | AAS | (SEQ ID NO: 879) | NSLRAEDTAVYY | (SEQ ID |
| | (SEQ ID NO: 878) | | C | NO: 881) |
| | | | (SEQ ID NO: 880) | |
| Ab_21 | QVQLVQSGGGL | MHWVRQAPGK | GYVDSVKGRFTIS | WGLGTL |
| | VQPGGSLRLSC | GLEWVSG | RDNRNNKVYLQ | VTVSS |
| | ATS | (SEQ ID NO: 883) | MNNLRAEDTAVY | (SEQ ID |
| | (SEQ ID NO: 882) | | YC | NO: 885) |
| | | | (SEQ ID NO: 884) | |
| Ab_22 | QVQLVQSGGGV | MHWVRQAPGK | YYADSVKGRFTIS | WGQGTT |
| | VQPGRSLRLSC | GLEWLAV | RDKSKNTLYLQIN | VTVSS |
| | AAS | (SEQ ID NO: 887) | SLRAEDTAVYYC | (SEQ ID |
| | (SEQ ID NO: 886) | | (SEQ ID NO: 888) | NO: 889) |
| Ab_23 | EVQLVQSGAEV | ITWVRQAPGQGL | NYAQKFQGRVTI | WGQGTL |
| | KKPGSSVKVSC | EWMGR | TADTSVSTAWME | VTVSS |
| | RSS | (SEQ ID NO: 891) | LSSLESDDTAIYY | (SEQ ID |
| | (SEQ ID NO: 890) | | C | NO: 893) |
| | | | (SEQ ID NO: 892) | |
| Ab_24 | QVQLVQSGAEV | VSWVRQAPGQG | NYAQKFQGRITIT | WGQGTL |
| | KTPGSSVKVSC | LEWMGG | ADEATSTVYMAL | VTVSS |
| | KAS | (SEQ ID NO: 895) | SSLRSEDTAMYY | (SEQ ID |
| | (SEQ ID NO: 894) | | C | NO: 897) |
| | | | (SEQ ID NO: 896) | |
| Ab_25 | QVQLVQSGGGL | MSWVRQAPGKG | YYVDSVKGRFTIS | WGQGTT |
| | VQPGGSLRLSC | LEWVAN | RDNAKNSLYLQM | VTVSS |
| | AAS | (SEQ ID NO: 899) | NSLRAEDTAVYY | (SEQ ID |
| | (SEQ ID NO: 898) | | C | NO: 901) |
| | | | (SEQ ID NO: 900) | |
| Ab_26 | QVQLVQSGGGL | MHWVRQVPGK | GYADSVKGRFTIS | WGQGTM |
| | VQPGRSLRLSC | GLEWVSG | RDNAKNSLYLQM | VTVSS |
| | AAS | (SEQ ID NO: 903) | NSLRAEDTALYY | (SEQ ID |
| | (SEQ ID NO: 902) | | C | NO: 905) |
| | | | (SEQ ID NO: 904) | |
| Ab_27 | QVQLVQSGTEV | LHWVRQAPGQG | NYAQKFQGRVTL | WGQGTL |
| | KKPGASVKVSC | LEWMGW | TRDTSISTAYMEL | VTVSS |
| | KAS | (SEQ ID NO: 907) | SGLRSDDTAVYY | (SEQ ID |
| | (SEQ ID NO: 906) | | C | NO: 909) |
| | | | (SEQ ID NO: 908) | |
| Ab_28 | QVTLKESGPGL | WSWIRQSPGKGL | IYNPSLKSRLTMSI | WGQGTL |
| | VNPSETLSLTCT | EWIGY | DTSKNQLSLNLRS | VTVSS |
| | VS | (SEQ ID NO: 911) | LTAADTAVYYC | (SEQ ID |
| | (SEQ ID NO: 910) | | (SEQ ID NO: 912) | NO: 913) |
| Ab_38 | QVTLKESGPTL | VGWIRQPPGKAL | RYSPSLRSRLTITR | WGQGTL |
| | VKPTQTLTLTCT | EWLAL | DTSKNQVVLTVT | VTVSS |
| | FS | (SEQ ID NO: 990) | DMDPADTGTYYC | (SEQ ID |
| | (SEQ ID NO: 989) | | (SEQ ID NO: 991) | NO: 992) |
| wcS2- | EVQLVQSGGGV | MHWVRQAPGK | YYADSVKGRFTIS | WGQGTT |
| T4-E7 | VQPGRSLRLSC | GLEWVAV | RDNSKNTLYLQM | VTVSS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | QVQLV*SGGGL | MHWVRQAPGK | GYADSVKGRFTIS | WGQGTL |
| E1-A9 | VQPGRSLRLSC | GLEWVSG | RDNAKNSLYLQM | VTVSS |
| | AAS | (SEQ ID NO: ) | DSLRAEDTAVYY | (SEQ ID |
| | (SEQ ID NO: ) | | C | NO: ) |
| | | | (SEQ ID NO: ) | |
| wcS2- | QV*LVQSGGGV | MHWVRQAPGK | YYADSVKGRFTIS | WGQGTL |
| T4-H8 | VQPGRSLRLSC | GLEWVAV | RDNSKNTLYLQM | VTVSS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | QVQLVQSGGGV | MHWVRQAPGK | YYADSVKGRFTIS | WGQGTL |
| T3-F5 | VQPGRSLRLSC | GLEWVAV | RDNAKNSLYLQM | VTVSS |
| | AAS | (SEQ ID NO: ) | NSLRDEDTAVYY | (SEQ ID |
| | (SEQ ID NO: ) | | C | NO: ) |
| | | | (SEQ ID NO: ) | |
| wcS2- | EVQLVQSGGGV | MHWVRQAPGK | YYADSVKGRFTIS | WGQGTL |
| E2-C1 | VQPGRSLRLSC | GLEWVAV | RDNSKNTLYLQM | VTVSS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | QVQLVQSGGGV | MHWVRQAPGK | YYADSVKGRFTIS | WGQGTL |
| T4-C9 | VQPGRSLRLSC | GLEWVAV | RDNSKNTLYLQM | VTVSS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | QVQLVQSGGGV | MHWVRQAPGK | YYADSVKGRFTIS | WGQGTL |
| T3-B9 | VQPGRSLRLSC | GLEWVAV | RDNSKNTLYLQM | VTVSS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | QV*LVQSGAEV | IAWVRQLPGQGL | RYSPSFQGQVTIS | WGQGTT |
| E3-C8 | KKPGESLKISCK | EWMGI | ADKSINTAYLQW | VTVSS |
| | GS | (SEQ ID NO: ) | SRLKASDTAKYY | (SEQ ID |
| | (SEQ ID NO: ) | | C | NO: ) |
| | | | (SEQ ID NO: ) | |
| wcS2- | QVELVQSGAEV | MHWVRQAPGQ | KYAEKFQGWVT | WGQGTM |
| T4-F8 | KEPGASVKVSC | GLEWMGW | MTRDTSISTAYM | VTVSS |
| | KDS | (SEQ ID NO: ) | ELSRLKSDDTALY | (SEQ ID |
| | (SEQ ID NO: ) | | YC | NO: ) |
| | | | (SEQ ID NO: ) | |
| wcS2- | EVQLVQSGGGV | MHWVRQAPGK | YYADSVKGRFTIS | WGQGTL |
| T3-F1 | VQPGRSLRLSC | GLEWVAV | RDNSKNTLYLQM | VTVSS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | QVQLVQSGGGV | MHWVRQAPGK | YYADSVKGRFTIS | WGKGTL |
| E2-B1 | VQPGRSLRLSC | GLEWVAV | RDNSKNTLYLQM | VTVSS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | QVQLQESGGGV | MHWVRQAPGK | YYADSVKGRFTIS | WGQGTL |
| T2-G3 | VQPGRSLRLSC | GLEWVAV | RDNSKNTLYLQM | VTVSS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | QV*LVQSGAEV | IAWVRQLPGQGL | RYSPSFQGQVTIS | WGQGTT |
| T1-A6 | KKPGESLKISCK | EWMGI | ADKSINTAYLQW | VTVSS |
| | GS | (SEQ ID NO: ) | SRLKASDTAKYY | (SEQ ID |
| | (SEQ ID NO: ) | | C | NO: ) |
| | | | (SEQ ID NO: ) | |
| wcS2- | EV*LVQSGGGV | MHWVRQAPGK | YYADSVKGRFTIS | WGQGTT |
| T4-D4 | VQPGRSLRLSC | GLEWVAV | RDNSKNTLYLQM | ATASS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | EVQLVQSGGGV | MHWVRQAPGK | YYADSVKGRFTIS | WGQGTL |
| T2-D10 | VQPGRSLRLSC | GLEWVAV | RDNSKNTLYLQM | VTVSS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | QVQLVQSGGGV | MHWVRQAPGK | YYADSVKGRFTIS | WGQGTL |
| T1-G9 | VQPGRSLRLSC | GLEWVAV | RDNSKNTLYLQM | VTVSS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | QVQLVQSGGGV | MHWVRQAPGK | YYADSVKGRFTIS | WGQGTL |
| E3-H7 | VQPGRSLRLSC | GLEWVAG | RDNSKNTLYLQM | VTVSS |
| | AAS | (SEQ ID NO: ) | NSLRAEDTAVYY | (SEQ ID |
| | (SEQ ID NO: ) | | C | NO: ) |
| | | | (SEQ ID NO: ) | |
| wcS2- | QVTLKESGPTL | VGWIRQPPGKAL | RYSPSLKSRLTISK | WGQGTM |
| T2-C11 | VKPTQTLTLTCT | EWLAL | DTSRNQVVLTMT | VTVSS |
| | FS | (SEQ ID NO: ) | NMDPADTGTYYC | (SEQ ID |
| | (SEQ ID NO: ) | | (SEQ ID NO: ) | NO: ) |
| SARS2- | QVQLVQSGGGL | MHWVRQAPGK | YYADSVQGRITIS | WGQGTL |
| R3-G2- | VKPGGSLRLSCS | GLEWVAV | RDNSKNTLYLQM | VTVSS |
| P1- | AS | | NSLRAEDTAVYF | |
| H9_PelB- | | | C | |
| F_2020- | | | | |
| 06- | | | | |
| 19_D08 | | | | |
| SARS2- | QVQLVQSGGGV | MHWVRQAPGK | YYADSVKGRFTIS | WGQGTL |
| R3-G3- | VQPGRSLRLSC | GLEWVAV | RDNSKNTLYLQM | VTVSS |
| P1- | AAS | | NSLRAEDTAVYY | |
| F8_PelB- | | | C | |
| F_2020- | | | | |
| 06- | | | | |
| 19_F11 | | | | |
| SARS2- | QVQLVQSGGGV | MHWVRQAPGK | YYADSVKGRFTIS | WGQGTL |
| R3-G2- | VQPGRSLRLSC | GLEWVAV | RDNSKNTLYLQM | VTVSS |
| P1- | AAS | | NSLRTEDTAVYY | |
| B4_PelB- | | | C | |
| F_2020- | | | | |
| 06- | | | | |
| 19_B02 | | | | |
| SARS2- | QVQLVQSGGGV | MHWVRQAPGK | YYADSVKGRFTIS | WGQGTT |
| R3-G3- | VQPGRSLRLSC | GLEWVAV | RDNSKNTLYLQM | VTVSS |
| P1- | AAS | | NSLRAEDTAVYY | |
| B2_PelB- | | | C | |
| F_2020- | | | | |
| 06- | | | | |
| 19_A08 | | | | |
| SARS2- | QVQLQESGGGV | MHWVRQAPGK | YYADSVKGRFTIS | WGQGTM |
| R3-G3- | VQPGRALRLSC | GLEWVAV | RDNSKNTLYLQM | VTVSS |
| P1- | RAS | | NSLRAEDTAVYY | |
| B3_PelB- | | | C | |
| F_2020- | | | | |
| 06- | | | | |
| 19_B08 | | | | |
| SARS2- | *VQLVQSGAEV | ISWVRQAPGQG | NYAQKFQGRVTI | WGQGTM |
| R3-G1- | KKPGSSVKVSC | LEWMGG | TADKSTSTAYME | VTVSS |
| P3- | KAS | | LSSLRSEDTAVYY | |
| E8_PelB- | | | C | |
| F_2020- | | | | |
| 06- | | | | |
| 19_H04 | | | | |
| SARS2- | EVQLVQSGAEV | ISWVRQAPGQG | NYAQKLQGRVT | WGQGTT |
| R3-G1- | KKPGASVKVSC | LEWMGW | MTTNTSTNTAYM | VTVSS |
| P2- | KAS | | ELRSLRSDDTAG | |
| D7_PelB- | | | YYW | |
| F_2020- | | | | |
| 06- | | | | |
| 19_H09 | | | | |
| SARS2- | QVTLKESGPTL | VGWIRQPPGKA | RYSPSLKSRLTIT | WGQGTL |
| R3-G1- | VKPTQTLTLTCT | LEWLAL | KDTSKNQVVLTM | VTVSS |
| P1- | FS | | TNMDPVDTATYY | |
| C6_PelB- | | | C | |
| F_2020- | | | | |
| 06- | | | | |
| 19_H02 | | | | |
| SARS2- | QVQLVQSGGGL | MHWVRQAPGK | YYAYSVQGRFTIS | RGRGTPG |
| R3-T-P2- | VQPGGSLRLSCS | GLEWVEV | KNNSKNTLYLQM | TASS |
| E12_ | AS | | NSLKAEDTAVYF | |
| PelB- | | | C | |
| F_2020- | | | | |
| 06- | | | | |
| 19_D07 | | | | |
| SARS2- | QVQLVQSGGGV | MHWVRQAPGK | YYADSVKGRFTIS | WGQGTM |
| R3-G2- | VQPGRSLRLSC | GLEWVAV | RDNSKNTLYLQM | VTVSS |
| P1- | AAS | | NSLRVEDTAVYY | |
| D12_ | | | C | |
| PelB- | | | | |
| F_2020- | | | | |
| 06- | | | | |
| 19_H04 | | | | |
| SARS2- | EVQLVQSGGGV | MHWVRQAPGK | YYADSVKGRFTIS | WGQGTL |
| R3-G1- | VQPGRSLRLSC | GLEWVAV | RDNSKNTLYLQM | VTVSS |
| P2- | AAS | | NSLRAEDTAVYY | |
| E8_PelB- | | | C | |
| F_2020- | | | | |
| 06- | | | | |
| 19_E10 | | | | |
| SARS2- | EVQLVQSGGGV | MHWVRQAPGK | YYADSVKGRFTIS | WGQGTT |
| R3-G1- | VQPGRSLTLSCA | GLEWVAV | RDNSKNTLYLQM | VTVSS |
| P1- | AS | | NSLRAEDTAVYY | |
| A10_ | | | C | |
| PelB- | | | | |
| F_2020- | | | | |
| 06- | | | | |
| 19_H01 | | | | |
| SARS2- | QVQLVQSGGGV | MHWVRQAPGK | YYADSVKGRFTIS | WGQGTL |
| R3-G1- | VQPGRSLRLSC | GLEWVAL | RDNAKNSLYLQM | VTVSS |
| P3- | AAS | | NSLRAEDTAVYY | |
| B5_PelB- | | | C | |
| F_2020- | | | | |
| 06- | | | | |
| 19_D02 | | | | |
| SARS2- | QVQLVQSGTEV | ISWVRQAPGQG | NYAQKFQGRVTI | WGQGTT |
| R3-G3- | RQPGASVKVSC | LEWMGG | TADESTSTAYME | VTVSS |
| P1- | KAS | | LSSLRSEDTAVYY | |
| A2_PelB- | | | C | |
| F_2020- | | | | |
| 06- | | | | |
| 19_B07 | | | | |
| SARS2- | EVQLVQSGGGV | MHWVRQAPGK | YYADSVKGRFTIS | WGKGTL |
| R3-G3- | VQPGRSLRLSC | GLEWVAV | RDNSKNTLYLQM | VTVSS |
| P1- | AAS | | NSLRAEDTAVYY | |
| D1_PelB- | | | C | |
| F_2020- | | | | |
| 06- | | | | |
| 19_G09 | | | | |
| SARS2- | QVQLVQSGGGV | LHWVRQAPGKG | YYADSVKGRFTIS | WGQGTM |
| R3-G1- | VRPGRSLRLSCA | LEWVAV | RDNAKNSLYLQM | VTVSS |
| P2- | AS | | NSLRAEDTAVYY | |
| D4_PelB- | | | C | |
| F_2020- | | | | |
| 06- | | | | |
| 19_G09 | | | | |
| SARS2- | EVQLVQSGGGV | MHWVRQAPGK | YYADSVKGRFTIS | WGQGTL |
| R3-G1- | VQPGRSLRLSC | GLEWVAV | RDNSKNTLYLQM | VTVSS |
| P1- | AAS | | NSLRAEDTAIYYC | |
| B6_PelB- | | | | |
| F_2020- | | | | |
| 06- | | | | |
| 19_E02 | | | | |
| SARS2- | QVTLKESGPAL | VGWIRQPPGKAL | RYSPSLKSRLTIT | WGQGTL |
| R3-G3- | VKPTQTLTLTCT | EWLAL | KDTSKNQVVLTM | VTVSS |
| P1- | FS | | TNMDPVDTATYY | |
| G8_PelB- | | | C | |
| F_2020- | | | | |
| 06- | | | | |
| 25_A05 | | | | |
| SARS2- | QVTLKESGPTL | VGWIRQPPGKAL | RYSPSLKSRLTIT | WGQGTT |
| R3-G1- | VKPTQTLTLTCT | EWLAL | KDTSKNQVVLTM | VTVSS |
| P1- | LS | | TNMDPVDTATYY | |
| F11_ | | | C | |
| PelB- | | | | |
| F_2020- | | | | |
| 06- | | | | |
| 25_B05 | | | | |
| SARS2- | QVQLVQSGGGV | MHWVRQAPGK | HYADSVKGRFTIS | WGQGTL |
| R3-T1- | VQPGRSRRLSCT | GLEWVST | RDNSKNTLYLQM | VTVSS |
| P3- | AS | | KSLRAEDTATYY | |
| F4_PelB- | | | C | |
| F_2020- | | | | |
| 06- | | | | |
| 20_C03 | | | | |
| SARS2- | QVQLVQSGGGL | MSWVRQAPGKG | YYADSVKGRFTIS | WGQGTL |
| R3-G1- | VQPGGSLRLSC | LEWVSA | RDNSKNTLYLQM | VTVSS |
| P2- | AAS | | NSLRAEDTGVYY | |
| B8_PelB- | | | C | |
| F_2020- | | | | |
| 06- | | | | |
| 25_C06 | | | | |
| SARS2- | QVQLVQSGGGV | MHWVRQAPGK | YYADSVKGRFTIS | WGQGTL |
| R3-G1- | VQPGRSLRLSC | GLEWVAV | RDNSKNTLYLQM | VTVSS |
| P2- | AAS | | NSLRAEDTAVYY | |
| D3_PelB- | | | C | |
| F_2020- | | | | |
| 06- | | | | |
| 25_C12 | | | | |
| SARS2- | EVQLVQSGGGV | MHWVRQAPGK | YYADSVKGRFTIS | WGQGTL |
| R3-G1- | VQPGRSLRLSC | GLEWVSY | RDNAKNSLYLQM | VTVSS |
| P4- | AAS | | NSLRAEDTAVYY | |
| C5_PelB- | | | C | |
| F_2020- | | | | |
| 06- | | | | |
| 25_C07 | | | | |
| SARS2- | QVQLVQSGAEV | FSWVRQAPGQG | DYAQNFQGRVT | WGQGTL |
| R3-G1- | KKPGASVKVSC | LEWMGW | MTTDTSTNTAYM | VTVSS |
| P2- | KAS | | ELRSLRSDDTAV | |
| D6_PelB- | | | YYC | |
| F_2020- | | | | |
| 06- | | | | |
| 25_D02 | | | | |
| SARS2- | EVQLVQSGAEV | MHWVRQAPGQ | SYAQKFQGRVTM | WGQGTT |
| R3-G1- | KKPGASVKVSC | GLEWMGI | TRDTSTSTVYME | VTVSS |
| P1- | KAS | | LSSLRSEDTAVYY | |
| B7_PelB- | | | C | |
| F_2020- | | | | |
| 06- | | | | |
| 25_A03 | | | | |
| 95_PelB- | QVQLVQSGGGV | MHWVRQAPGK | YYADSVKGRFTIS | WGQGTT |
| F_2020- | VQPGRSLRLSC | GLEWVAV | RDNSKKMLYLQ | VTVSS |
| 06- | AAS | | MNSLTAEDTAVY | |
| 25_G11 | | | YC | |
| 73_PelB- | QVQLVQSGGGL | MNWVRQAPGK | YYADSVKGRFTIS | WGQGTL |
| F_2020- | VQPGGSLRLSC | GLEWVAV | RDNSKNTLYLQM | VTVSS |
| 06- | AAS | | NSLRAEDTAVYY | |
| 25_F09 | | | C | |
| SARS2- | QVQLVQSGGGL | MHWVRQAPGK | YYADSVKGRFTIS | WGQGTL |
| R3-G3- | VKPGGSLRLSC | GLEWVAV | RDNSKNTLYLQM | VTVSS |
| P1- | AAS | | NSLRAEDTAVYY | |
| G9_PelB- | | | C | |
| F_2020- | | | | |
| 06- | | | | |
| 25_B05 | | | | |
| SARS2- | QVQLVQSGAEV | MHWVRQAPGQ | NYAQKFQGRVT | WGQGTT |
| R3-G1- | KKPGASVKVSC | GLEWMGR | MTRDTSISTAYM | VTVSS |
| P3- | KAS | | ELSRLRSDDTAV | |
| G7_PelB- | | | YYC | |
| F_2020- | | | | |
| 06- | | | | |
| 25_H01 | | | | |
| SARS2- | QVQLQQSGAEV | VSWVRQAPGQG | NYAQRFQGRVTI | WGKGTT |
| R3-G1- | KKPGSSVKVSC | PEWMGR | TADESTNTAYME | VTVSS |
| P4- | KAS | | LSSLRSEDTAVYY | |
| A2_PelB- | | | C | |
| F_2020- | | | | |
| 06- | | | | |
| 25_A06 | | | | |
| SARS2- | EVQLVQSGAEV | ISWVRQAPGQGL | NYAQKLQGRVT | WGQGTT |
| R3-G3- | KKPGASVKVSC | EWMGW | MTTDTSTSTAYM | VTVSS |
| P1- | KAS | | ELRSLRSDDTAV | |
| G1_PelB- | | | YYC | |
| F_2020- | | | | |
| 06- | | | | |
| 25_F04 | | | | |
| SARS2- | QVQLVQSGGGV | MHWVRQAPGK | YYADSVKGRFTIS | WGQGTL |
| R3-G3- | VQPGRSLRLSC | GLEWVAV | RDNSKNTLYLQM | VTVSS |
| P1- | AAS | | NSLRDEDTAVYY | |
| F1_PelB- | | | C | |
| F_2020- | | | | |
| 06- | | | | |
| 25_C04 | | | | |
| SARS2- | QVQLQESGAGL | WSWIRQPPGKGL | NYNPSFQSRVAM | WGQGTL |
| R3-G1- | LRPSETLSLTCA | EWIGE | SRDTPKNQFSLKL | VTVSS |
| P3- | VY | | SSVTAADTAVYY | |
| H10_ | | | C | |
| PelB- | | | | |
| F_2020- | | | | |
| 06- | | | | |
| 25_H10 | | | | |
| 85_PelB- | QVQLV*SGAEV | VGWVRQKPGKG | RKNPSFEGQVTM | WGQGTL |
| F_2020- | KKPGESLKISCQ | LEWMGI | SVDKSLHSVYLH | GTVSS |
| 06- | GS | | WTSLKVSDTAKY | |
| 25_G10 | | | YC | |
| SARS2- | QVQLVQSGAEV | INWVRQATGQG | GYAQSFQGRVTF | WGQGTT |
| R3-G1- | KKPGASVKVSC | LEWMGW | TRDTSINTAYMEL | VTVSS |
| P4- | KAS | | SSLRSEDTAVYYC | |
| A3_PelB- | | | | |
| F_2020- | | | | |
| 06- | | | | |
| 25_B06 | | | | |
| SARS2- | QVQLVQSGAEV | ISWVRQMPGKG | NYSPSFQGHVTIT | WGQGTM |
| R3-G1- | KKSGESLEISCK | LEWMGR | VDKSTGAAYLH | VTVSS |
| P3- | GS | | WSSLKASDTGMY | |
| H6_PelB- | | | YC | |
| F_2020- | | | | |
| 06- | | | | |
| 25_H08 | | | | |
| SARS2- | EVQLVQSGAEV | IGWVRQMPGKG | IYSPSFQGQVTISA | WGQGTM |
| R3-G3- | KKPGESLRISCK | LEWMGV | DKSISTAYLQWSS | VTVSS |
| P1- | GS | | LKASDTAIYYC | |
| G5_PelB- | | | | |
| F_2020- | | | | |
| 06- | | | | |
| 25_H04 | | | | |
| SARS2- | EVQLVESGGGQ | MHWVRQAPGK | YYADSLKGRFNIS | WGHGTL |
| R3-G1- | VKPGGSLRISCA | GLEWVSS | RDNAKKLLYLQL | VTVSS |
| P4- | AS | | SSLSAEDTALYFC | |
| C10_ | | | | |
| PelB- | | | | |
| F_2020- | | | | |
| 06- | | | | |
| 25_F07 | | | | |
| SARS2- | QVQLVQSGAEV | ISWVRQAPGQGL | NYAQKLQGRVT | WGQGTL |
| R3-G1- | KKPGSSVKVSC | ELMGW | MTTDTSTSTAYM | VTVSS |
| P1- | KAS | | ELRSLRSGDTAV | |
| F10_ | | | YYC | |
| PelB- | | | | |
| F_2020- | | | | |
| 06- | | | | |
| 25_B04 | | | | |
| SARS2- | EVQLVESGPGL | WSWIRQHPGKG | YYNPSLKSRVTIS | WGQGTM |
| R3-T1- | VKPSQTLSLTCT | LEWIGY | VDTSKNQFSLKLS | VTVSS |
| P4- | VS | | SVTAADTAVYYC | |
| H4_PelB- | | | | |
| F_2020- | | | | |
| 06- | | | | |
| 20_H04 | | | | |
| SARS2- | QVQLVQSGGGV | MHWVRQAPGK | YYADSVKGRFTIS | WGQGTL |
| R3-G2- | VRPGRSLRLSCA | GLEWVAV | RDNAKNSLYLQM | VTVSS |
| P1- | AS | | NSLRAEDTAVYY | |
| E8_PelB- | | | C | |
| F_2020- | | | | |
| 06- | | | | |
| 25_A02 | | | | |
| SARS2- | QVQLVQSGGGL | MHWVRQAPGK | YYADSVKGRFTIS | WGQGTL |
| R3-G1- | VQPGGSLRLSC | GLEWVAV | RDNSKNTLYLQM | VTVSS |
| P1- | AAS | | NSLRAEDTAVYY | |
| C3_PelB- | | | C | |
| F_2020- | | | | |
| 06- | | | | |
| 25_A07 | | | | |
| SARS2- | EVQLVQSGGGL | MHWVRQAPGK | DYAESVKGRFTIS | WGQGTL |
| R3-G3- | VQPGGSLRLPCS | GLEHISI | RDNSKNTLYLQM | VTVSS |
| P1- | AS | | TSLREEDTAVYY | |
| G2_PelB- | | | C | |
| F_2020- | | | | |
| 06- | | | | |
| 25_G04 | | | | |
| SARS2- | QVQLVQSGAEV | ISWVRQAPGQGP | NYAQRFQGRVTI | WGKGTL |
| R3-G1- | KKPGSSAKVSC | EWMGR | TADESTNTAYME | VTVSS |
| P4- | KAS | | LSSLRSEDTAVYY | |
| D1_PelB- | | | C | |
| F_2020- | | | | |
| 06- | | | | |
| 25_A08 | | | | |

TABLE 64B
Light chain (V_{L}) framework regions (FRs) of the COVID-19 antibodies.

| Seq ID | FR1-IMGT | FR2-IMGT | FR3-IMGT | FR4-IMGT |
|---|---|---|---|---|
| S1-R3- | LPVLTQPPS.AS | VNWYQQLPGTA | QRPSGVPDRFSG | FGSGTKVT |
| T1-H7 | GTPGQRVTISCS | PKLLIY | SKSGTSASLAISG | VL |
| (Ab12) | GS | (SEQ ID NO: 543) | LQSEDEADYYC | (SEQ ID |
| | (SEQ ID NO: 542) | | (SEQ ID NO: 544) | NO: 545) |
| RBD-R3- | NFMLTQPHS.VS | VQWYQQRPGSS | QRPSGVPDRFSG | FGGGTKLT |
| E1-G7 | ESPGKTVTISCT | PTTVIY | SIDSSSNSASLTIS | VL |
| (AB2-10) | RS | (SEQ ID NO: 547) | GLTTEDEADYYC | (SEQ ID |
| | (SEQ ID NO: 546) | | (SEQ ID NO: 548) | NO: 549) |
| S1-RBD- | QPGLTQPPS.AS | VTWYQQLPGTA | QRPSGVPDRFSA | FGGGTKLT |
| R3-E1- | GTPGQRVTISCS | PKLLIY | SRSGTSASLAITG | VL |
| D8 | GS | (SEQ ID NO: 551) | LQAEDEADYYC | (SEQ ID |
| (Ab_3) | (SEQ ID NO: 550) | | (SEQ ID NO: 552) | NO: 553) |
| S1-RBD- | NFMLTQPHS.VS | VQWYQQRPGSS | LRPSGVPDRFSG | FGGGTKLT |
| R3-T1- | ESPGNTVTISCT | PTTVIF | SIDSSSNSASLTIS | VL |
| C7 | RT | (SEQ ID NO: 555) | GLKTEDEADYY | (SEQ ID |
| | (SEQ ID NO: 554) | | C | NO: 557) |
| | | | (SEQ ID NO: 556) | |
| RBD-R3- | NFMLTQPHS.VS | VQWYRQRPGSA | QRPAGVPDRFSG | FGTGTKLT |
| T1-F4 | ESPGKTVIISCTR | PTTVIY | SVDSSSNSASLTI | VL |
| (Ab2-2) | T | (SEQ ID NO: 559) | TGLKTEDEADY | (SEQ ID |
| | (SEQ ID NO: 558) | | YC | NO: 561) |
| | | | (SEQ ID NO: 560) | |
| RBD-R3- | SYELTQPPS.VSE | VSWYQHLPGKA | RLPSGVSDRFSG | FGGGTKLT |
| E1-A5 | APRQRVTISCSG | PKLLIY | SKSGTSASLAISG | VL |
| | S | (SEQ ID NO: 563) | LRSEDEADYYC | (SEQ ID |
| | (SEQ ID NO: 562) | | (SEQ ID NO: 564) | NO: 565) |
| S1-RBD- | NFMLTQPHS.VS | VQWYQQRPGSS | QRPSGVPDRFSG | FGGGTKLT |
| R3-T1- | ESPGKTVTISCT | PTTVIY | SIDSSSNSASLTIS | VL |
| F5 | RS | | GLKAEDEADYY | (SEQ ID |
| (Ab2-7) | (SEQ ID NO: 566) | (SEQ ID NO: 567) | C | NO: 569) |
| | | | (SEQ ID NO: 568) | |
| S1-R3- | QPVLTQPPSASG | VNWYQQLPGTA | QRPSGVPDRFSD | FGTGTKVT |
| T1-A12 | TPGQRVTISCSG | PKLLIY | SKSGTSASLAISG | VL |
| (Ab_5) | S | (SEQ ID NO: 571) | LQSEDEADYFC | (SEQ ID |
| | (SEQ ID NO: 570) | | (SEQ ID NO: 572) | NO: 573) |
| S1-R3- | QSVLTQPPSASG | VSWYQQYPGKA | KRPSGVPDRFSG | FGGGTKLT |
| T1-A6 | SPGQSVTISCTG | PKLMIY | SKSGNTASLTVS | VL |
| (Ab_4) | T | (SEQ ID NO: 575) | GLRAEDEADYY | (SEQ ID |
| | (SEQ ID NO: 574) | | C | NO: 577) |
| | | | (SEQ ID NO: 576) | |
| S1-RBD- | QPGLTQPPSVSV | VHWYQQKAGQ | DRPSEIPERFSGS | FGTGTKVT |
| R3-T1- | APGKTARITCG | APVLVVY | NSGNTATLTISR | VL |
| A5 | GN | (SEQ ID NO: 579) | AEVGDEADYYC | (SEQ ID |
| | (SEQ ID NO: 578) | | (SEQ ID NO: 580) | NO: 581) |
| S1-RBD- | SSELTQDPAVSV | AGWYQQKPGQA | KRPSGIPDRFSAS | FGGGTKLT |
| R3-T1- | VLGQAVRITCQ | PVLVTY | TSGNTASLTITG | VL |
| B3 | GD | (SEQ ID NO: 583) | AQADDEADYYC | (SEQ ID |
| (Ab_1) | (SEQ ID NO: 582) | | (SEQ ID NO: 584) | NO: 585) |
| S1-RBD- | QSALTQPPSVSG | VSWYQQPPGKA | NRPSGVSNRFSG | FGTGTKVT |
| R3-T1- | SPGQSVTISCTG | PKLMIY | SKSGNTASLTISG | VL |
| E7 | T | (SEQ ID NO: 587) | LQAEDEADYYC | (SEQ ID |
| | (SEQ ID NO: 586) | | (SEQ ID NO: 588) | NO: 589) |
| S1-RBD- | QSVVTQPPSVSG | VQWYQQLPGTA | NRPSGVPDRFSA | FGGGTKVT |
| R3-T1- | APGQRVTISCTG | PKLLIW | SKSGTSASLAITG | VL |
| F9 | S | (SEQ ID NO: 591) | LQAEDEADYFC | (SEQ ID |
| | (SEQ ID NO: 590) | | (SEQ ID NO: 592) | NO: 593) |
| S1-R3- | NFMLTQPHSVS | VQWYQQRPGSS | DRPSGVPDRFSG | FGGGTQLT |
| T1-C2 | ESPGKTVTISCT | PTTVIY | SIDSSSNSASLTIS | VL |
| (Ab_6) | RS | (SEQ ID NO: 595) | GLKPEDEADYY | (SEQ ID |
| | (SEQ ID NO: 594) | | C | NO: 597) |
| | | | (SEQ ID NO: 596) | |
| RBD-R3- | QTVVTQEPAVS | ASWYQQKPGQA | KRPSGIPDRFSGS | FGGGTKLT |
| E1-B3 | VALGQTVRITC | PVLVVY | TSGNTASLTITG | VL |
| | QGD | (SEQ ID NO: 599) | AQAEDEADYYC | (SEQ ID |
| | (SEQ ID NO: 598) | | (SEQ ID NO: 600) | NO: 601) |
| RBD-R3- | EIVLTQSPATLS | LAWYQQRPGQA | TRATAIPARFSGS | FGGGTKVE |
| T1-B5 | LSPGERATLSCR | PRLLIY | GSGTEFTLTISRL | IN |
| | AS | (SEQ ID NO: 603) | EPEDFATYYC | (SEQ ID |
| | (SEQ ID NO: 602) | | (SEQ ID NO: 604) | NO: 605) |
| RBD-R3- | QSALTQPPSASG | VSWYQQHPGKA | KRPPGVPDRFSG | FGGGTKLT |
| T1-H3 | SPGQSVTISCTG | PKLLIY | SKSGNTASLTVS | VL |
| (Ab_2) | T | (SEQ ID NO: 607) | GLQAEDEADYY | (SEQ ID |
| | (SEQ ID NO: 606) | | C | NO: 609) |
| | | | (SEQ ID NO: 608) | |
| S1-R3- | QPGLTQPPSVSK | AAWLQQHQGHP | NRPPGISERFSAS | FGGGTKLS |
| T1-C4 | GLRQTATLTCT | PKLLSY | RSGNTASLTITGL | VL |
| (Ab_8) | GN | (SEQ ID NO: 611) | QPEDEADYYC | (SEQ ID |
| | (SEQ ID NO: 610) | | (SEQ ID NO: 612) | NO: 613) |
| S1-RBD- | NFMLTQPHSVS | VQWYQQRPGSA | QRPSGVPDRFSG | FGGGTKLT |
| R3-T1- | ESPGKTVTISCT | PTTVIY | SIDSSSNSASLTIS | VL |
| B12 | GS | (SEQ ID NO: 615) | GLKTEDEADYY | (SEQ ID |
| | (SEQ ID NO: 614) | | C | NO: 617) |
| | | | (SEQ ID NO: 616) | |
| S1-RBD- | NFMLTQPHSVS | VQWYQQRPGSA | QRPSGVPDRFSG | FGGGTKLT |
| R3-T1- | ESPGKTVTISCT | PTTVIY | SIGSSSNSASLTIS | VL |
| G5 | GS | (SEQ ID NO: 619) | GLKTEDEADYY | (SEQ ID |
| | (SEQ ID NO: 618) | | C | NO: 621) |
| | | | (SEQ ID NO: 620) | |
| S1-RBD- | QPGLTQPPSVSV | VHWYQQKPGRA | GRPSGIPERFSGS | FATGTKVS |
| R3-T1- | APGQTARISCGG | PVLVVY | NSGNTATLTVSR | IL |
| E2 (Ab7) | N | (SEQ ID NO: 623) | VEAGDEADYYC | (SEQ ID |
| | (SEQ ID NO: 622) | | (SEQ ID NO: 624) | NO: 625) |
| RBD-R3- | QPGLTQPASVS | VSWYQQHPGKA | KRPSGISNRFSGS | FGGGTKLT |
| T1-F7 | GSPGQSVTISCT | PKLMIY | KSGNTASLTISGL | VL |
| | GT | (SEQ ID NO: 627) | QAEDEADYFC | (SEQ ID |
| | (SEQ ID NO: 626) | | (SEQ ID NO: 628) | NO: 629) |
| S1-RBD- | QPGLTQPPSASG | VNWYQQLPGTA | QRPSGVPDRFSG | FGGGTKLT |
| R3-T1- | TPGQRVTISCSG | PKLLIY | SKSGTSASLAISG | VL |
| G1 | S | (SEQ ID NO: 631) | LRSEDEADYYC | (SEQ ID |
| | (SEQ ID NO: 630) | | (SEQ ID NO: 632) | NO: 633) |
| S1-RBD- | QPVLTQPHSVSE | VQWYQQRPGSA | QRPSGVPDRFSG | FGGGTKLT |
| R3-T1- | SPGKTVTISCTR | PTTVIY | SIDSSSNSASLIIS | VL |
| C2 | S | (SEQ ID NO: 635) | GLMTEDEADYY | (SEQ ID |
| | (SEQ ID NO: 634) | | C | NO: 637) |
| | | | (SEQ ID NO: 636) | |
| S1-RBD- | QPGLTQPPSVSV | VHWYQQKAGQ | GRPSGIPERFSGS | FGPGTRLS |
| R3-T1- | APGQTATITCGG | APVLVVY | NSGNTATLTISR | VL |
| H8 | D | (SEQ ID NO: 639) | VEAGDEADYYC | (SEQ ID |
| | (SEQ ID NO: 638) | | (SEQ ID NO: 640) | NO: 641) |
| S1-RBD- | NFMLTQPHSVS | VQWYQQRPGSA | QRPSGVPDRFSG | FGGGTKLT |
| R3-E1- | ESPGKTVLISCT | PTTVIY | SIDSSSNSASLTIS | VL |
| E8 | RS | (SEQ ID NO: 643) | GLKTEDEADYY | (SEQ ID |
| | (SEQ ID NO: 642) | | C | NO: 645) |
| | | | (SEQ ID NO: 644) | |
| RBD-R3- | EIVLTQSPATLS | LPWYQQKPGQA | TRATGIPPRFSGS | FDGGTNVE |
| T1-H2 | VSPGERATLSCR | PRLLMY | GSGTEFSLTISSL | IK |
| | AS | (SEQ ID NO: 647) | QSEDFAVYYC | (SEQ ID |
| | (SEQ ID NO: 646) | | (SEQ ID NO: 648) | NO: 649) |
| S1-RBD- | NFMLTQPHSVS | VQWYQQRPGSF | QRPSGVPYRFSG | FGGGTKLT |
| R3-T1- | ESPGKTITISCTR | PITVIY | SIDRSSNSAALTI | VL |
| B7 | T | (SEQ ID NO: 651) | SDLKTEDEADYY | (SEQ ID |
| | (SEQ ID NO: 650) | | C | NO: 653) |
| | | | (SEQ ID NO: 652) | |
| S1-RBD- | QSALTQPASVS | VSWYQQHPGKA | KRPSGVPDRFSG | FGTGTKVT |
| R3-E1- | GSPGQSITISCTG | PKLMIY | SKSGNTASLTISG | VL |
| E5 | T | (SEQ ID NO: 655) | LQAEDEADYYC | (SEQ ID |
| | (SEQ ID NO: 654) | | (SEQ ID NO: 656) | NO: 657) |
| S1-R3- | SYELTQPPSVSV | VHWYQQKSGQA | DRPSGIPERFSGS | FGGGTKLT |
| T1-H6 | APGQTARITCG | PVLVVY | NSGNTATLTISR | VL |
| | GD | (SEQ ID NO: 659) | VEAGDEADYYC | (SEQ ID |
| | (SEQ ID NO: 658) | | (SEQ ID NO: 660) | NO: 661) |
| S1-RBD- | SSELTQDPAVSV | ASWYQQKPGQA | NRPSGIPDRFSGS | FGGGTKLA |
| R3-E1- | ALGQTVKITCQ | PVRVIY | SSGNTASLTITGA | VL |
| C6 | GD | (SEQ ID NO: 663) | QAEDEADYYC | (SEQ ID |
| | (SEQ ID NO: 662) | | (SEQ ID NO: 664) | NO: 665) |
| S1-RBD- | QSALTQPPSASG | VSWYQHHPDKA | NRPSGVSSRFSG | FGTGTEVT |
| R3-E1- | SPGQSLTISCTG | PKLLIY | SKSGNTASLTISG | PR |
| F2 | T | (SEQ ID NO: 667) | LQAEDEADYYC | (SEQ ID |
| | (SEQ ID NO: 666) | | (SEQ ID NO: 668) | NO: 669) |
| S1-RBD- | LPVLTQPPSVSV | VHWYQQKPGQA | DRPSGIPERFSGS | FGGGTTLT |
| R3-T1- | APGQTASITCGG | PLLVIY | SSGNTATLTISRV | VL |
| C3 | D | (SEQ ID NO: 671) | EAGDEADYYC | (SEQ ID |
| | (SEQ ID NO: 670) | | (SEQ ID NO: 672) | NO: 673) |
| S1-RBD- | QSALTQPRSVSG | VSWYQQHPGKA | KRPSGVPDRFSG | FGGGTKLT |
| R3-T1- | SPGQSVTISCTG | PKLIIY | SKSGNTASLTVS | VL |
| G12 | T | (SEQ ID NO: 675) | GLQAEDEADYY | (SEQ ID |
| | (SEQ ID NO: 674) | | C | NO: 677) |
| | | | (SEQ ID NO: 676) | |
| S1-RBD- | QSVLTQPPSASG | VSWYQQHPGKA | RRPSGVSSRFSGS | FGTGTKVT |
| R3-E1- | SPGQSVTISCTG | PKLMIY | KSGNTASLTISGL | VL |
| F1 | T | (SEQ ID NO: 679) | QAEDEADYYC | (SEQ ID |
| | (SEQ ID NO: 678) | | (SEQ ID NO: 680) | NO: 681) |
| S1-RBD- | NFMLTQPHSVS | VQWYQQRPGSA | QRPSGVPDRFSG | FGGGTKLT |
| R3-E1- | ESPGKTVTISCT | PTTVIY | SIDSSSNSASLTIS | VI |
| H8 | GS | (SEQ ID NO: 683) | GLKTEDEADYY | (SEQ ID |
| | (SEQ ID NO: 682) | | C | NO: 685) |
| | | | (SEQ ID NO: 684) | |
| S1-RBD- | QPGLTQTPSPSG | VTWYQQLPGTA | QRPSGVPDRFSA | FGGGTKM |
| R3-T1- | TPGQRVTISCSG | PKLLIY | SRSGTSPSLAITG | TVL |
| F1 | S | (SEQ ID NO: 687) | LQAEDEADYYC | (SEQ ID |
| | (SEQ ID NO: 686) | | (SEQ ID NO: 688) | NO: 689) |
| S1-R3- | LPVLTQPPSASG | VNWYHQLPRTA | HRPSGVPDRFSG | FGSGTKVT |
| T1-B10 | TPGQRVTISCSG | PKLLIY | S*SGTSASLAITV | VL |
| | S | (SEQ ID NO: 691) | IQSEDEADYYC | (SEQ ID |
| | (SEQ ID NO: 690) | | (SEQ ID NO: 692) | NO: 693) |
| RBD-R3- | LPVLTQPPSASG | VNWYHYLPRTA | HRASRVPDRFSG | FGSGTKVT |
| E1-D12 | TPGQRVTISCSG | PKLLIY | S*SGTSASLAITV | VL |
| | S | (SEQ ID NO: 695) | IQSEDEADYYC | (SEQ ID |
| | (SEQ ID NO: 694) | | (SEQ ID NO: 696) | NO: 697) |
| RBD-R3- | LPVLTQPPSASG | VNWYQQLPGTA | QRPSRVPDRFSG | FGSGTKSP |
| T1-C5 | TPGQRVTISCSG | PKLLIY | SKTGTSPSLAISV | SY |
| | S | (SEQ ID NO: 699) | LQSEDEADYYC | (SEQ ID |
| | (SEQ ID NO: 698) | | (SEQ ID NO: 700) | NO: 701) |
| S1-RBD- | QPVLTQPPSASG | VNWYQQLPGTA | QRPSGVPDRFSG | FGGGTKLT |
| R3-T1- | TPGQRVTISCSG | PKLLIY | SKSGTSASLAISG | VL |
| B4 | S | (SEQ ID NO: 703) | LQSEDEADYYC | (SEQ ID |
| | (SEQ ID NO: 702) | | (SEQ ID NO: 704) | NO: 705) |
| S1-RBD- | SSELTQDPAVSV | ASWYQQKPGQA | NRPSGIPDRFSGS | FGGGTKLT |
| R3-E1- | ALGQTVRITCQ | PVLVIY | SSGNTASLTITGA | VL |
| E7 | GD | (SEQ ID NO: 707) | QAEDEADYYC | (SEQ ID |
| | (SEQ ID NO: 706) | | (SEQ ID NO: 708) | NO: 709) |
| S1-RBD- | QPVLTQPPSASG | VNWYQQLPGTA | QRYPGVPDRFSG | FGGGTKLT |
| R3-T1- | TPGQRVTISCSG | PKLLIY | SKSGTSASLAISG | VL |
| C8 | S | (SEQ ID NO: 711) | LRSEDEADYYC | (SEQ ID |
| | (SEQ ID NO: 710) | | (SEQ ID NO: 712) | NO: 713) |
| S1-RBD- | QPGLTQPPSASG | VHWYQQLPGTA | QRPSGVPDRFSG | FGGGTKLT |
| R3-T1- | TPGQGVTISCSG | PKLLIY | SKSGTSASLAISG | VL |
| D7 | S | (SEQ ID NO: 715) | LRSEDEADYYC | (SEQ ID |
| | (SEQ ID NO: 714) | | (SEQ ID NO: 716) | NO: 717) |
| RBD-R3- | NFMLTQPHSVS | VQWLQQRLGSA | QRPSGVPDRFSG | FGGGTKLT |
| E1-F5 | ESPGKTVTISCT | PTTVIY | SIDSSSNSASLTIS | VL |
| | GS | (SEQ ID NO: 719) | GLKTEDEADYY | (SEQ ID |
| | (SEQ ID NO: 718) | | C | NO: 721) |
| | | | (SEQ ID NO: 720) | |
| Ab_13 | NFMLTQPHSVS | VQWYQQRPGSA | QRPSGVPDRFSG | FGGGTKLT |
| | ESPGKTVTISCT | PTTVIY | SIDSSSNSASLTIS | VL |
| | RS | (SEQ ID NO: 915) | GLKTEDEADYY | (SEQ ID |
| | (SEQ ID NO: | | C | NO: 917) |
| | 914) | | (SEQ ID NO: 916) | |
| Ab_14 | QSVLTQPPSASG | VSWYQHHPGKA | KRPSGVPDRFSG | FGGGTKLT |
| | SPGQSVTISCTG | PKLIIY | SKSGNTASLTVS | VL |
| | T | (SEQ ID NO: 919) | GLQADDEADYY | (SEQ ID |
| | (SEQ ID NO: | | C | NO: 921) |
| | 918) | | (SEQ ID NO: 920) | |
| Ab_15 | NFMLTQPHSVS | VQWYQQRPGSA | QRPSGVPDRFSG | FGTGTKVT |
| | ESPGKTVTISCT | PTTVIY | SIDRSSNSASLTIS | VL |
| | RS | (SEQ ID NO: 923) | GLTPDDEADYY | (SEQ ID |
| | (SEQ ID NO: 922) | | C | NO: 925) |
| | | | (SEQ ID NO: 924) | |
| Ab_16 | QSVLTQPPSVSG | VHWYQQVPGAA | NRPSGVPDRFSG | FGTGTKVT |
| | SPGQRVTMSCT | PRLLIY | SKSGTSASLTITG | VL |
| | GS | (SEQ ID NO: 927) | LQAEDEADYYC | (SEQ ID |
| | (SEQ ID NO: 926) | | (SEQ ID NO: 928) | NO: 929) |
| Ab_17 | QSALTQPASVS | VSWYQQHPGKA | KRPSGVPDRFSG | FGGGTKLT |
| | GSPGQSITISCTG | PKVMIY | SKSGNTASLTISG | VL |
| | T | (SEQ ID NO: 931) | LQAEDEADYYC | (SEQ ID |
| | (SEQ ID NO: 930) | | (SEQ ID NO: 932) | NO: 933) |
| Ab_18 | NFMLTQPHSMS | VQWYQQRPGSA | QRPSGVPDRFSG | FGGGTRLT |
| | GSAGKTVTVSCI | PTTVIY | SIDSSSNSASLTIS | VL |
| | RS | (SEQ ID NO: 935) | GLKTEDEADYY | (SEQ ID |
| | (SEQ ID NO: 934) | | C | NO: 937) |
| | | | (SEQ ID NO: 936) | |
| Ab_19 | QPGLTQPPAAS | VSWFKQFPETAP | HRPSGVPDRVSG | FGSGTKVT |
| | GTPGQRVTVSC | RLLIS | SKSGTSASLTISG | VL |
| | SGA | (SEQ ID NO: 939) | LQSDDEADYYC | (SEQ ID |
| | (SEQ ID NO: 938) | | (SEQ ID NO: 940) | NO: 941) |
| Ab_20 | NFMLTQPHSVS | VQWYQQRPGSA | QRPSGVP.DRFSG | FGGGTKLT |
| | ESPGKTVTISCT | PTTVIY | SIDSSSNSASLLIS | VL |
| | GS | (SEQ ID NO: 943) | GLKTEDEADYY | (SEQ ID |
| | (SEQ ID NO: 942) | | C | NO: 945) |
| | | | (SEQ ID NO: 944) | |
| Ab_21 | QSALTQPRSVSG | VSWYQQHPGKA | KRPSGVPDRFSG | FGGGTKLT |
| | SPGQSVTISCTG | PKLIIY | SKSGNTASLTVS | VL |
| | T | (SEQ ID NO: 947) | GLQAEDEADYY | (SEQ ID |
| | (SEQ ID NO: 946) | | C | NO: 949) |
| | | | (SEQ ID NO: 948) | |
| Ab_22 | NFMLTQPHSVS | VQWYRQRPGSA | QRPAGVPDRFSG | FGTGTKLT |
| | ESPGKTVIISCTR | PTTVIY | SVDSSSNSASLTI | VL |
| | T | (SEQ ID NO: 951) | TGLKTEDEADY | (SEQ ID |
| | (SEQ ID NO: 950) | | YC | NO: 953) |
| | | | (SEQ ID NO: 952) | |
| Ab_23 | QSALTQPPSASG | VSWYQQVPGKA | NRPSGVSSRFSG | FGGGTKLT |
| | SPGQSVTISCTG | PKLIIY | SKSGNTASLTISG | VL |
| | T | (SEQ ID NO: 955) | LQAEDEADYYC | (SEQ ID |
| | (SEQ ID NO: 954) | | (SEQ ID NO: 956) | NO: 957) |
| Ab_24 | QPVLTQPPSASG | VDWYHQVPGTA | ERSSGVPDRFSA | FGGGTKLT |
| | TPGQRVSISCSG | PQLLIY | SRSGNTASLTIIG | VL |
| | S | (SEQ ID NO: 959) | LQPEDEADYYC | (SEQ ID |
| | (SEQ ID NO: 958) | | (SEQ ID NO: 960) | NO: 961) |
| Ab_25 | NFMLTQPHSVS | VQWYQQRPGSA | QRPSGVPDRFSG | FGGGTKLT |
| | ESPGKTVTISCT | PTTVIY | SIGSSSNSASLTIS | VL |
| | GS | (SEQ ID NO: 963) | GLKTEDEADYY | (SEQ ID |
| | (SEQ ID NO: 962) | | C | NO: 965) |
| | | | (SEQ ID NO: 964) | |
| Ab_26 | LPVLTQPPSVSV | VHWYQQKPGQA | DRPSGIPERFSGS | FGGGTTLT |
| | APGQTASITCGG | PLLVIY | SSGNTATLTISRV | VL |
| | D | (SEQ ID NO: 967) | EAGDEADYYC | (SEQ ID |
| | (SEQ ID NO: 966) | | (SEQ ID NO: 968) | NO: 969) |
| Ab_27 | QSALTQPPSVSG | VSWYQQPPGKA | NRPSGVSNRFSG | FGTGTKVT |
| | SPGQSVTISCTG | PKLMIY | SKSGNTASLTISG | VL |
| | T | (SEQ ID NO: 971) | LQAEDEADYYC | (SEQ ID |
| | (SEQ ID NO: 970) | | (SEQ ID NO: 972) | NO: 973) |
| Ab_28 | NFMLTQPHSVS | VQWYQQRPGSA | QRPSGVPDRFSG | FGGGTKLT |
| | ESPGKTVTISCT | PTTVIY | SIDSSSNSASLTIS | VL |
| | GS | (SEQ ID NO: 975) | GLKTEDEADYY | (SEQ ID |
| | (SEQ ID NO: 974) | | C | NO: 977) |
| | | | (SEQ ID NO: 976) | |
| Ab_38 | EIVLTQSPATLS | LAWYQQKPGQA | TRATGIPARFSGS | FGGGTKVE |
| | VSPGERATLSCR | PRLLMY | GSGTEFSLTISSL | IK |
| | AS | (SEQ ID NO: 994) | QSEDFAVYYC | (SEQ ID |
| | (SEQ ID NO: 993) | | (SEQ ID NO: 995) | NO: 996) |
| wcS2- | NFMLTQPHSVS | VQWYQQRPGSA | QRPSGVSGRFSG | FGGGTKLT |
| T4-E7 | ESPGKTVTMSC | PTTVIY | SIDSSSNSASLTIS | VL |
| | TRS | (SEQ ID NO: ) | GLQPEDEADYY | (SEQ ID NO: ) |
| | (SEQ ID NO: ) | | C | |
| | | | (SEQ ID NO: ) | |
| wcS2- | QPVLTQPPSASG | VSWYQQHPDKA | KRPSGVPDRFSG | FGGGTKVT |
| E1-A9 | SPGQSVTISCTG | PKLLIY | SKSGNTASLTVS | VL |
| | T | (SEQ ID NO: ) | GLQADDEADYY | (SEQ ID NO: ) |
| | (SEQ ID NO: ) | | C | |
| | | | (SEQ ID NO: ) | |
| wcS2- | ETTLTQSPATLS | LAWYQQKPGQA | SRATGIPDRFSGS | FGGGTKVE |
| T4-H8 | LSPGERATLSCR | PRLLIY | GSGTDFTLTISRL | IK |
| | AS | (SEQ ID NO: ) | EPEDFAVYYC | (SEQ ID NO: ) |
| | (SEQ ID NO: ) | | (SEQ ID NO: ) | |
| wcS2- | QPVLTQPRSVSG | VSWYQHHPGKA | ERPSGVSSRFSGS | FGTGTRVA |
| T3-F5 | SPGQSVTISCTG | PKLMIY | KSGNTASLTISGL | VL |
| | T | (SEQ ID NO: ) | QGEDEADYFC | (SEQ ID NO: ) |
| | (SEQ ID NO: ) | | (SEQ ID NO: ) | |
| wcS2- | QSVLTQPPSASG | VSWYQQHPGKA | KRPSGVPDRFSG | FGTGTKVT |
| E2-C1 | SPGQSVTISCTG | PKPMIY | SKSGNTASLTVS | VL |
| | T | (SEQ ID NO: ) | GLQAEDEADYY | (SEQ ID NO: ) |
| | (SEQ ID NO: ) | | C | |
| | | | (SEQ ID NO: ) | |
| wcS2- | EIVLTQSPATLS | LAWYQQKPGQA | SRANGIPDRFSGS | FGGGTKLE |
| T4-C9 | LSPGERATLSCR | PRLLIN | GSGTDFTLTITRL | IK |
| | AS | (SEQ ID NO: ) | EPEDFAVYFC | (SEQ ID NO: ) |
| | (SEQ ID NO: ) | | (SEQ ID NO: ) | |
| wcS2- | SSELTQDPAVSV | ASWYQQKPGQA | NRPSGIPDRFSGS | FGGGTKLT |
| T3-B9 | ALGQTVRITCQ | PLLVIY | SSGNTASLTITGA | VL |
| | GD | (SEQ ID NO: ) | QAEDEADYYC | (SEQ ID NO: ) |
| | (SEQ ID NO: ) | | (SEQ ID NO: ) | |
| wcS2- | QSVLTQPLSASG | VNWYQQVPGTA | RRPSGVPDRFSG | FGTGTKVT |
| E3-C8 | TPGHRVTISCSG | PKLLIY | SKSGTSAALAIS | VL |
| | S | (SEQ ID NO: ) | GLQSEDEADYY | (SEQ ID NO: ) |
| | (SEQ ID NO: ) | | C | |
| | | | (SEQ ID NO: ) | |
| wcS2- | NFMLTQPHSVS | VQWYQQRPGSA | QRPSGVPDRFSG | FGGGTKLT |
| T4-F8 | ESPGKTVTISCT | PTAVIF | SIDSSSNSASLTIS | VL |
| | RS | (SEQ ID NO: ) | GLRTEDEADYY | (SEQ ID NO: ) |
| | (SEQ ID NO: ) | | C | |
| | | | (SEQ ID NO: ) | |
| wcS2- | LPVLTQPPSASG | VNWYQQLPGTA | QRPSGVPDRFSG | FGGGTNLA |
| T3-F1 | TPGQRVTISCSG | PKLLIY | SKSGTSASLAISG | VL |
| | S | (SEQ ID NO: ) | LRSEDEADYYC | (SEQ ID NO: ) |
| | (SEQ ID NO: ) | | (SEQ ID NO: ) | |
| wcS2- | SSELTQDPAVSV | ASWYQQKPGQA | NRPSGIPDRFSGS | FGTGTKVT |
| E2-B1 | ALGQTVRITCQ | PVLVIY | SSGNTASLTITGA | VL |
| | GD | (SEQ ID NO: ) | QAEDEADYYC | (SEQ ID NO: ) |
| | (SEQ ID NO: ) | | (SEQ ID NO: ) | |
| wcS2- | SYELTQPPSVSQ | AAWLQQHQGHP | IRPSGISERLSAST | FGEGTKLT |
| T2-G3 | GLRQTATLTCT | PKLLSY | SGNTASLTITGL | VL |
| | GN | (SEQ ID NO: ) | QPEDEADYYC | (SEQ ID NO: ) |
| | (SEQ ID NO: ) | | (SEQ ID NO: ) | |
| wcS2- | QSVLTQPLSASG | VNWYQQVPGTA | RRPSGVPDRFSG | FGTGTKVT |
| T1-A6 | TPGHRVTISCSG | PKLLIY | SKSGTSAALAIS | VL |
| | S | (SEQ ID NO: ) | GLQSEDEADYY | (SEQ ID NO: ) |
| | (SEQ ID NO: ) | | C | |
| | | | (SEQ ID NO: ) | |
| wcS2- | NFMLTHPHSVS* | VQWYQKRPGSA | QRPAGVSGRFSG | FGGGTNLT |
| T4-D4 | SPGKTATMSCT | PTTVIY | SIDSSSNSASLTIS | VL |
| | RS | (SEQ ID NO: ) | AVHPEDEADYY | (SEQ ID NO: ) |
| | (SEQ ID NO: ) | | C | |
| | | | (SEQ ID NO: ) | |
| wcS2- | HFVLTQPPSASE | DDCCHQRPPGAP | HKPSAGSADLFA | FGAGTKVT |
| T2-D10 | SPGKTVTMSCT | PTAMN | AFDSASTSALIAF | VL |
| | GT | (SEQ ID NO: ) | SVLHADDDDDY | (SEQ ID NO: ) |
| | (SEQ ID NO: ) | | CC | |
| | | | (SEQ ID NO: ) | |
| wcS2- | ETTLTQSPGTLS | LAWYQQKPGQA | NRAPGIPARFSGS | FGPGTKVH |
| T1-G9 | LSPGERATLSCR | PRLLIY | GSGTDFTLTISSL | IK |
| | AS | (SEQ ID NO: ) | EPEDFAVYYC | (SEQ ID NO: ) |
| | (SEQ ID NO: ) | | (SEQ ID NO: ) | |
| wcS2- | EIVLTQSPATLA | LAWYQQRPGQA | SRANGIPYRFSGS | SAERTKME |
| E3-H7 | LSPGERATLSCR | PRLLIN | GSGTDFTLTITRL | IK |
| | DS | (SEQ ID NO: ) | EPEDFAVYFC | (SEQ ID NO: ) |
| | (SEQ ID NO: ) | | (SEQ ID NO: ) | |
| wcS2- | SSELTQDPAVSV | ANWYQQKPGQA | ERPSGIPERFSGS | FGGGTKLT |
| T2-C11 | ALGQTVRITCQ | PILVMS | SSGTRVTLTISGV | VL |
| | GD | (SEQ ID NO: ) | QAEDEADYYC | (SEQ ID NO: ) |
| | (SEQ ID NO: ) | | (SEQ ID NO: ) | |
| SARS2- | QSALTQPASVS | VSWYQQHPGKA | KRPSGVPDRFSG | FGTGTKVA |
| R3-G2- | GSPGQSITISCTG | PKLMIY | SKSGNTASLTVS | VL |
| P1- | T | | GLQAEDEADYY | |
| H9_PelB- | | | C | |
| F_2020- | | | | |
| 06- | | | | |
| 19_D08 | | | | |
| SARS2- | QSALTQPASVS | VSWYQQHPGKA | NRPSGVSNRFSG | FGAGTKVT |
| R3-G3- | GSPGQSITISCTG | PKLMIS | SKSGNTASLTISG | VL |
| P1- | T | | LQAEDEADYYC | |
| F8_PelB- | | | | |
| F_2020- | | | | |
| 06- | | | | |
| 19_F11 | | | | |
| SARS2- | EIVLTQSPATLS | LAWYQQKPGQA | TRATGIPARFSGS | FGQGTRVE |
| R3-G2- | VSPGERATLSCR | PRLLIY | GSGTEFTLTVSR | IR |
| P1- | AS | | LEPEDFAVYYC | |
| B4_PelB- | | | | |
| F_2020- | | | | |
| 06- | | | | |
| 19_B02 | | | | |
| SARS2- | QSVLTQPPSASG | VNWYQQLPGTA | QRPSGVPDRFSG | FGGGTNLA |
| R3-G3- | TPGQRVTISCSG | PKLLIY | SKSGTSVSLAISG | VL |
| P1- | S | | LQSEDEADYYC | |
| B2_PelB- | | | | |
| F_2020- | | | | |
| 06- | | | | |
| 19_A08 | | | | |
| SARS2- | SYELTQPPSVSV | VHWYQQKPGQA | DRPPGIPERFSGS | FGTGTKVT |
| R3-G3- | APGKTANMTCG | PVLVVY | NSGNTATLTISR | VL |
| P1- | GN | | VEVGDEADYYC | |
| B3_PelB- | | | | |
| F_2020- | | | | |
| 06- | | | | |
| 19_B08 | | | | |
| SARS2- | SSELTQDPAVSV | ASWYQQKPGQA | NRPSGIPDRISGS | FGTGTKVT |
| R3-G1- | ALGQTVRITCQ | PVLVIY | SSGNTASLTITGA | VL |
| P3- | GD | | QAEDEADYYC | |
| E8_PelB- | | | | |
| F_2020- | | | | |
| 06- | | | | |
| 19_H04 | | | | |
| SARS2- | QSGLTQPPSVSG | VHWYQQLPGTA | NRPSGVPDRFSG | FGGGTKLT |
| R3-G1- | APGQRVTISCTG | PKLIIY | SKSGTSASLAITG | VL |
| P2- | S | | LQAEDEADYYC | |
| D7_PelB- | | | | |
| F_2020- | | | | |
| 06- | | | | |
| 19_H09 | | | | |
| SARS2- | SYELTQPPSVSV | AYWYQQRPGQA | ERPSGIPERFSGS | FGGGTKVT |
| R3-G1- | SPGQTARITCSG | PVLVIY | SSGTTVTLTISGV | VL |
| P1- | D | | QAEDEADYYC | |
| C6_PelB- | | | | |
| F_2020- | | | | |
| 06- | | | | |
| 19_H02 | | | | |
| SARS2- | HTVLTHPAPAA | VSWYQQHPGKA | NRPSGVPDRFSG | S*TGTEVA |
| R3-T-P2- | AYPGQTITISCS | PTLVIY | SIYGNTDSLTVS | FI |
| E12_ | AT | | AGVEDEDDYYC | |
| PelB- | | | | |
| F_2020- | | | | |
| 06- | | | | |
| 19_D07 | | | | |
| SARS2- | DIVMTQTPSTLS | VAWYQQKPGKV | RLEPGVPSRFSGS | FGQGTKV |
| R3-G2- | ASVGDRVTISCR | PELLMY | GSGTEFTLTISNL | DIK |
| P1- | AS | | QPEDFATYYC | |
| D12_ | | | | |
| PelB- | | | | |
| F_2020- | | | | |
| 06- | | | | |
| 19_H04 | | | | |
| SARS2- | NFMLTQPHSVS | VQWYQQRPGSS | QRPSEVPDRFSG | FGGGTKLT |
| R3-G1- | ESPGKTVTISCT | PTAVIY | SIDISSNSASLTIS | VL |
| P2- | RS | | GLKTEDEADYY | |
| E8_PelB- | | | C | |
| F_2020- | | | | |
| 06- | | | | |
| 19_E10 | | | | |
| SARS2- | ETTLTQSPGTLS | LAWYQQKPGQA | TRATGIPARFSGS | FGQGTKLE |
| R3-G1- | LSPGERATLSCR | PRLLIY | GSGTDFTLTISRL | IK |
| P1- | AS | | EPEDFAVYFC | |
| A10_ | | | | |
| PelB- | | | | |
| F_2020- | | | | |
| 06- | | | | |
| 19_H01 | | | | |
| SARS2- | QSALTQPPSASG | VSWYQQPPGTA | NRPSGVPDRFSG | FGTGTKVT |
| R3-G1- | SPGQSVTISCTG | PKLMIY | SKSGNTASLTISG | VL |
| P3- | T | | LQAEDEADYYC | |
| B5_PelB- | | | | |
| F_2020- | | | | |
| 06- | | | | |
| 19_D02 | | | | |
| SARS2- | NFMLTQPHSVS | VQWYQQRPGSS | RRPSGVPDRFSG | FGGGTKLT |
| R3-G3- | GSPGKTVTISCT | PTTVIY | SVDRSSNSASLTI | VI |
| P1- | RD | | SGLDTEDEADYY | |
| A2_PelB- | | | C | |
| F_2020- | | | | |
| 06- | | | | |
| 19_B07 | | | | |
| SARS2- | QPVLTQPPSVSG | VQWYQQVPGTA | NRPSGVPDRFSG | FGTGTKVN |
| R3-G3- | APGQSVTISCIG | PKLLIY | SKSGTSASLAISG | VL |
| P1- | S | | LRSEDEADYFC | |
| D1_PelB- | | | | |
| F_2020- | | | | |
| 06- | | | | |
| 19_G09 | | | | |
| SARS2- | QPGLTQPPSVSK | AAWLQQHQGHP | NRPSGISERFSAS | FGGGSRLT |
| R3-G1- | DLRQTATLTCT | PKLLSY | TSGNTASLTITGL | VL |
| P2- | GN | | QPEDEADYYC | |
| D4_PelB- | | | | |
| F_2020- | | | | |
| 06- | | | | |
| 19_G09 | | | | |
| SARS2- | QPVLTQPPSASG | VNWYQQLPGTA | HRPSGVPDRFSG | FGGGTKLT |
| R3-G1- | TPGQRVTISCSG | PKLLIY | SKSGNTASLTISG | VL |
| P1- | G | | LQAEDEADYYC | |
| B6_PelB- | | | | |
| F_2020- | | | | |
| 06- | | | | |
| 19_E02 | | | | |
| SARS2- | QPVLTQPPSVSV | ASWYQQKPGQS | KRPSGIPERFSGS | FGTGTKVT |
| R3-G3- | SPGQTASITCSG | PVLVIY | NSGNTATLTISET | VL |
| P1- | D | | QAMDEADYYC | |
| G8_PelB- | | | | |
| F_2020- | | | | |
| 06- | | | | |
| 25_A05 | | | | |
| SARS2- | SYELTQPPSVSV | AYWYRQRPGQA | ERPSGIPERFSGS | FGGGTKLT |
| R3-G1- | SPGQTATITCSG | PVLVIY | SSGTTVTLTISGV | VL |
| P1- | D | | QAEDEADYYC | |
| F11_ | | | | |
| PelB- | | | | |
| F_2020- | | | | |
| 06- | | | | |
| 25_B05 | | | | |
| SARS2- | QSVLTQPPSASG | VSWYQQHPGKA | KRPSGVPDRFSG | FGTGTEVT |
| R3-T1- | SPGQSVTISCTG | PKVLIY | SKSGNTASLTVS | VL |
| P3- | T | | GLQAEDEADYY | |
| F4_PelB- | | | C | |
| F_2020- | | | | |
| 06- | | | | |
| 20_C03 | | | | |
| SARS2- | QSVLTQPPSASG | VSWYQHHPGKA | KRPSGVPDRFSG | FGTGTKVT |
| R3-G1- | SPGHSVTISCTG | PKLMIY | SKSGNTASLSVS | VL |
| P2- | T | | GLQAEDEADYY | |
| B8_PelB- | | | C | |
| F_2020- | | | | |
| 06- | | | | |
| 25_C06 | | | | |
| SARS2- | NFMLTQPHSVS | VQWYQQRPGSS | QRPSGVPDRFSG | FGGGTKLT |
| R3-G1- | ESPGKTVAISCT | PTTVIY | SIDSSSNSASLTIS | VL |
| P2- | RS | | GLKTEDEADYY | |
| D3_PelB- | | | C | |
| F_2020- | | | | |
| 06- | | | | |
| 25_C12 | | | | |
| SARS2- | LPMLTQPPSMS | VNWYQQVPGTA | QRPSGVPDRFSG | IGGGTKLT |
| R3-G1- | GTPGQRVTISCS | PKVLIY | SKSGTSASLAISG | VL |
| P4- | GS | | LRSEDEADYSC | |
| C5_PelB- | | | | |
| F_020- | | | | |
| 06- | | | | |
| 25_C07 | | | | |
| SARS2- | SYELTQPPSVSK | ASWLQHHQGHP | NRPSGISERFSAS | FGTGTKVT |
| R3-G1- | GLRQTATLTCT | PKLLSY | RSGNTASLTITGL | VI |
| P2- | GN | | QPEDEADYYC | |
| D6_PelB- | | | | |
| F_2020- | | | | |
| 06- | | | | |
| 25_D02 | | | | |
| SARS2- | SYELTQPPSVSV | VSWYQQKPGHS | KRPSGIPERFSGS | FGGGTKLA |
| R3-G1- | SPGQTATITCSG | PLLVIY | NSGNRAILTINGT | VL |
| P1- | D | | QALDEADYYC | |
| B7_PelB- | | | | |
| F_2020- | | | | |
| 06- | | | | |
| 25_A03 | | | | |
| 95_PelB- | ETTLTQSPGTLS | LAWYQQKPGQA | NRATGIPDRFSG | FGQGTKLE |
| F_2020- | LSPGERATLSCR | PRLLIY | SGSGTDFTLTISS | IN |
| 06- | AS | | LQPEDFATYYC | |
| 25_G11 | | | | |
| 73_PelB- | DIVMTQSPATLS | LAWYQQRPGQA | TRATDIPDRFTGS | FGQGTKVE |
| F_2020- | VSPGERATLSCR | PRLLIS | GSGTDFTLTISSL | SK |
| 06- | AS | | EPEDFAVYYC | |
| 25_F09 | | | | |
| SARS2- | QSVLTQPPSASG | VSWYQQHPGKA | KRPSGVPDRFSA | FGGGTTLT |
| R3-G3- | SPGQSVTVSCTG | PKLLIH | SKSGNTASLTISG | VI |
| P1- | T | | LQPEDEGDYFC | |
| G9_PelB- | | | | |
| F_2020- | | | | |
| 06- | | | | |
| 25_B05 | | | | |
| SARS2- | QPVLTQPPSVSK | TAWLQQHQGHP | NRPSGVSERFSA | FGGGTKLT |
| R3-G1- | DLRQTATLTCT | PKLVSY | SRSGNTASLTITG | VL |
| P3- | GN | | LQAEDEADYYC | |
| G7_PelB- | | | | |
| F_2020- | | | | |
| 06- | | | | |
| 25_H01 | | | | |
| SARS2- | NFMLTQPHSVS | VQWYQQRPGSA | QRPSGVPDRFSG | FGGGTKLT |
| R3-G1- | ESPGKTVTISCT | PTTVIY | SIDSSSKTASLIIS | VP |
| P4- | RS | | GLETEDEADYYC | |
| A2_PelB- | | | | |
| F_2020- | | | | |
| 06- | | | | |
| 25_A06 | | | | |
| SARS2- | QSVLTQPPSVSG | VHWYQQLPGTA | NRPSGVPDRFSG | FGGGTKLT |
| R3-G3- | APGQRVTISCTG | PKLIIY | SKSGTSASLAITG | VL |
| P1- | S | | LQAEDEADYYC | |
| G1_PelB- | | | | |
| F_2020- | | | | |
| 06- | | | | |
| 25_F04 | | | | |
| SARS2- | QSALTQPASVS | VSWYQQHPASA | NRPSGVSNRFSG | FGTGTKVT |
| R3-G3- | GSPGQSITISCTG | PKLIIY | SESGDTASLTISG | VL |
| P1- | T | | LRAEDEADYFC | |
| F1_PelB- | | | | |
| F_2020- | | | | |
| 06- | | | | |
| 25_C04 | | | | |
| SARS2- | LPVLTQPPSASE | VNWYQQLPGTA | QRPSGVPDRFFG | FGGGTKLT |
| R3-G1- | TPGQRVTISCSG | PKLLIY | SKSGTSASLAISG | VL |
| P3- | G | | LQSEDEADYYC | |
| H10_ | | | | |
| PelB- | | | | |
| F_2020- | | | | |
| 06- | | | | |
| 25_H10 | | | | |
| 85_PelB- | QPVLTQPPSVSG | VHWYQQLPGTA | QRPSGVPDRFSG | FGGGTKVT |
| F_2020- | APGQSVTISCTG | PKLLIY | SKSGTSASLAISG | VL |
| 06- | S | | LQSEDEADYYC | |
| 25_G10 | | | | |
| SARS2- | QSVLTQPPSASG | VYWYQRLPGTA | QRPSGVPDRFSG | FGTGTKVT |
| R3-G1- | TPGGRVTISCSG | PKLLIY | SKSGTSASLAISG | VI |
| P4- | S | | LQSEDEADYFC | |
| A3_PelB- | | | | |
| F_2020- | | | | |
| 06- | | | | |
| 25_B06 | | | | |
| SARS2- | SSELTQDPAVSV | ANWYQQKSGQA | IRPSIPDRFSGSSS | VRRRIKLT |
| R3-G1- | ALGQTVRIICQG | PILVMY | GNTASLTITGAQ. | VL |
| P3- | D | | EDEADYYC | |
| H6_PelB- | | | | |
| F_2020- | | | | |
| 06- | | | | |
| 25_H08 | | | | |
| SARS2- | QSALTQPASVS | VSWYQQHPGKA | YRPSGISHRFSGS | FGTGTKVT |
| R3-G3- | GSPGQSITISCTG | PKLMIY | KSGNTASLTISGL | VL |
| P1- | T | | QAEDEADYYC | |
| G5_PelB- | | | | |
| F_2020- | | | | |
| 06- | | | | |
| 25_H04 | | | | |
| SARS2- | SYELTQPPSVSV | AYWYQQRPGQA | ERSSGIPERFSGS | FGGGTKLT |
| R3-G1- | SPGQTASITCSG | PVLLIY | GSGTTVTLTING | VI |
| P4- | D | | VQAEDEADYYC | |
| C10_ | | | | |
| PelB- | | | | |
| F_2020- | | | | |
| 06- | | | | |
| 25_F07 | | | | |
| SARS2- | LPVLTQPPSVSG | VHWYQQLPGAA | NRPSGVPDRFSG | FGGGTRLT |
| R3-G1- | APGQRVAISCTG | PKLLIY | SKSGTSASLAISG | VL |
| P1- | S | | LRSEDEADYYC | |
| F10_ | | | | |
| PelB- | | | | |
| F_2020- | | | | |
| 06- | | | | |
| 25_B04 | | | | |
| SARS2- | QSVLTQPPSVSV | VHWYQQKAGQ | DRPSGIPERFSGS | FGGGTKLT |
| R3-T1- | APGQTARITCG | APVLVVH | NSGNTATLTISR | VL |
| P4- | GD | | VEAGDEADYYC | |
| H4_PelB- | | | | |
| F_2020- | | | | |
| 06- | | | | |
| 20_H04 | | | | |
| SARS2- | DIVMTQSPLSLP | LDWYLQKPGQS | NRASGVPDRFSG | FGQGTRLD |
| R3-G2- | VTPGEPASISCR | PQLLIH | SGSDTDFTLKISR | IK |
| P1- | SS | | VEAEDVGVYYC | |
| E8_PelB- | | | | |
| F_2020- | | | | |
| 06- | | | | |
| 2_A02 | | | | |
| SARS2- | QSVLTQPPSVSG | VHWYQQLPGTA | IRPSGVPDRFSAS | FGGGTKLT |
| R3-G1- | APGERVTFSCTG | PKLLIY | KSGTSASLAITGL | VL |
| P1- | T | | QSEDEGDYYC | |
| C3_PelB- | | | | |
| F_2020- | | | | |
| 06- | | | | |
| 25_A07 | | | | |
| SARS2- | QSVLTQPPSASG | VSWYQQHPGKA | KRPSGVPDRFSG | FGTGTEVT |
| R3-G3- | SPGQSVTISCTG | PKVLIY | SKSGNTASLTVS | VL |
| P1- | T | | GLQAEDEADYY | |
| G2_PelB- | | | C | |
| F_2020- | | | | |
| 06- | | | | |
| 25_G04 | | | | |
| SARS2- | NFMLTQPHSVS | VQWYQQRPGSA | QRPSGVPDRFSA | FGGGTKLT |
| R3-G1- | ESPGKTVTISCT | PTTVIY | SIDSSSNSASLTIS | VL |
| P4- | RS | | GLKAEDEADYY | |
| D1_PelB- | | | C | |
| F_2020- | | | | |
| 06- | | | | |
| 25_A08 | | | | |

The asterisks noted in the tables herein are read as a Q (glutamine) in the amino acid sequences described in the tables herein.

### Antibodies

As used herein, the term “antibody” can refer to immunoglobulin molecules and immunologically active portions of immunoglobulin (Ig) molecules, i.e., molecules that contain an antigen binding site that specifically binds (immunoreacts with) an antigen. By “specifically binds” or “immunoreacts with” is meant that the antibody reacts with one or more antigenic determinants of the desired antigen and does not react with other polypeptides. Antibodies can include, but are not limited to, polyclonal, monoclonal, and chimeric antibodies. In some embodiments, the antibodies described herein are directed to SARS-CoV2. For example, the antibodies described herein are directed to SARS-CoV2 having NCBI Reference Sequence:

NC_045512 (amino acid residues 1-7116; SEQ ID NO: 979):
MESLVPGFNEKTHVQLSLPVLQVRDVLVRGFGDSVEEVLSEARQHLKDGTCGLVEVEKGVLPQLEQP
YVFIKRSDARTAPHGHVMVELVAELEGIQYGRSGETLGVLVPHVGEIPVAYRKVLLRKNGNKGAGGH
SYGADLKSFDLGDELGTDPYEDFQENWNTKHSSGVTRELMRELNGGAYTRYVDNNFCGPDGYPLECI
KDLLARAGKASCTLSEQLDFIDTKRGVYCCREHEHEIAWYTERSEKSYELQTPFEIKLAKKEDTENG
ECPNFVFPLNSIIKTIQPRVEKKKLDGFMGRIRSVYPVASPNECNQMCLSTLMKCDHCGETSWQTGD
FVKATCEFCGTENLTKEGATTCGYLPQNAVVKIYCPACHNSEVGPEHSLAEYHNESGLKTILRKGGR
TIAFGGCVFSYVGCHNKCAYWVPRASANIGCNHTGVVGEGSEGLNDNLLEILQKEKVNINIVGDFKL
NEEIAIILASFSASTSAFVETVKGLDYKAFKQIVESCGNFKVTKGKAKKGAWNIGEQKSILSPLYAF
ASEAARVVRSIFSRTLETAQNSVRVLQKAAITILDGISQYSLRLIDAMMFTSDLATNNLVVMAYITG
GVVQLTSQWLTNIFGTVYEKLKPVLDWLEEKFKEGVEFLRDGWEIVKFISTCACEIVGGQIVTCAKE
IKESVQTFFKLVNKFLALCADSIIIGGAKLKALNLGETFVTHSKGLYRKCVKSREETGLLMPLKAPK
EIIFLEGETLPTEVLTEEVVLKTGDLQPLEQPTSEAVEAPLVGTPVCINGLMLLEIKDTEKYCALAP
NMMVTNNTFTLKGGAPTKVTFGDDTVIEVQGYKSVNITFELDERIDKVLNEKCSAYTVELGTEVNEF
ACVVADAVIKTLQPVSELLTPLGIDLDEWSMATYYLFDESGEFKLASHMYCSFYPPDEDEEEGDCEE
EEFEPSTQYEYGTEDDYQGKPLEFGATSAALQPEEEQEEDWLDDDSQQTVGQQDGSEDNQTTTIQTI
VEVQPQLEMELTPVVQTIEVNSFSGYLKLTDNVYIKNADIVEEAKKVKPTVVVNAANVYLKHGGGVA
GALNKATNNAMQVESDDYIATNGPLKVGGSCVLSGHNLAKHCLHVVGPNVNKGEDIQLLKSAYENFN
QHEVLLAPLLSAGIFGADPIHSLRVCVDTVRTNVYLAVFDKNLYDKLVSSFLEMKSEKQVEQKIAEI
PKEEVKPFITESKPSVEQRKQDDKKIKACVEEVTTTLEETKELTENLLLYIDINGNLHPDSATLVSD
IDITFLKKDAPYIVGDVVQEGVLTAVVIPTKKAGGTTEMLAKALRKVPTDNYITTYPGQGLNGYTVE
EAKTVLKKCKSAFYILPSIISNEKQEILGTVSWNLREMLAHAEETRKLMPVCVETKAIVSTIQRKYK
GIKIQEGVVDYGARFYFYTSKTTVASLINTLNDLNETLVTMPLGYVTHGLNLEEAARYMRSLKVPAT
VSVSSPDAVTAYNGYLTSSSKTPEEHFIETISLAGSYKDWSYSGQSTQLGIEFLKRGDKSVYYTSNP
TTFHLDGEVITFDNLKTLLSLREVRTIKVFTTVDNINLHTQVVDMSMTYGQQFGPTYLDGADVTKIK
PHNSHEGKTFYVLPNDDTLRVEAFEYYHTTDPSFLGRYMSALNHTKKWKYPQVNGLTSIKWADNNCY
LATALLTLQQIELKENPPALQDAYYRARAGEAANFCALILAYCNKTVGELGDVRETMSYLFQHANLD
SCKRVLNVVCKTCGQQQTTLKGVEAVMYMGTLSYEQFKKGVQIPCTCGKQATKYLVQQESPFVMMSA
PPAQYELKHGTFTCASEYTGNYQCGHYKHITSKETLYCIDGALLTKSSEYKGPITDVFYKENSYTTT
IKPVTYKLDGVVCTEIDPKLDNYYKKDNSYFTEQPIDLVPNQPYPNASFDNFKFVCDNIKFADDLNQ
LTGYKKPASRELKVTFFPDLNGDVVAIDYKHYTPSFKKGAKLLHKPIVWHVNNATNKATYKPNTWCI
RCLWSTKPVETSNSFDVLKSEDAQGMDNLACEDLKPVSEEVVENPTIQKDVLECNVKTTEVVGDIIL
KPANNSLKITEEVGHTDLMAAYVDNSSLTIKKPNELSRVLGLKTLATHGLAAVNSVPWDTIANYAKP
FLNKVVSTTTNIVTRCLNRVCTNYMPYFFTLLLQLCTFTRSTNSRIKASMPTTIAKNTVKSVGKFCL
EASFNYLKSPNFSKLINIIIWFLLLSVCLGSLIYSTAALGVLMSNLGMPSYCTGYREGYLNSTNVTI
ATYCTGSIPCSVCLSGLDSLDTYPSLETIQITISSFKWDLTAFGLVAEWFLAYILFTRFFYVLGLAA
IMQLFFSYFAVHFISNSWLMWLIINLVQMAPISAMVRMYIFFASFYYVWKSYVHVVDGCNSSTCMMC
YKRNRATRVECTTIVNGVRRSFYVYANGGKGFCKLHNWNCVNCDTFCAGSTFISDEVARDLSLQFKR
PINPTDQSSYIVDSVTVKNGSIHLYFDKAGQKTYERHSLSHFVNLDNLRANNTKGSLPINVIVEDGK
SKCEESSAKSASVYYSQLMCQPILLLDQALVSDVGDSAEVAVKMFDAYVNTFSSTFNVPMEKLKTLV
ATAEAELAKNVSLDNVLSTFISAARQGFVDSDVETKDVVECLKLSHQSDIEVTGDSCNNYMLTYNKV
ENMTPRDLGACIDCSARHINAQVAKSHNIALIWNVKDEMSLSEQLRKQIRSAAKKNNLPFKLTCATT
RQVVNVVTTKIALKGGKIVNNWLKQLIKVTLVFLFVAAIFYLITPVHVMSKHTDFSSEIIGYKAIDG
GVTRDIASTDTCFANKHADFDTWFSQRGGSYTNDKACPLIAAVITREVGFVVPGLPGTILRTTNGDE
LHFLPRVFSAVGNICYTPSKLIEYTDFATSACVLAAECTIFKDASGKPVPYCYDTNVLEGSVAYESL
RPDTRYVLMDGSIIQFPNTYLEGSVRVVTTFDSEYCRHGTCERSEAGVCVSTSGRWVLNNDYYRSLP
GVFCGVDAVNLLTNMFTPLIQPIGALDISASIVAGGIVAIVVTCLAYYFMRFRRAFGEYSHVVAFNT
LLFLMSFTVLCLTPVYSFLPGVYSVIYLYLTFYLTNDVSFLAHIQWMVMFTPLVPFWITIAYIICIS
TKHFYWFFSNYLKRRVVFNGVSFSTFEEAALCTFLLNKEMYLKLRSDVLLPLTQYNRYLALYNKYKY
FSGAMDTTSYREAACCHLAKALNDFSNSGSDVLYQPPQTSITSAVLQSGFRKMAFPSGKVEGCMVQV
TCGTTTLNGLWLDDVVYCPRHVICTSEDMLNPNYEDLLIRKSNHNFLVQAGNVQLRVIGHSMQNCVL
KLKVDTANPKTPKYKFVRIQPGQTFSVLACYNGSPSGVYQCAMRPNFTIKGSFLNGSCGSVGENIDY
DCVSFCYMHHMELPTGVHAGTDLEGNFYGPFVDRQTAQAAGTDTTITVNVLAWLYAAVINGDRWFLN
RFTTTLNDFNLVAMKYNYEPLTQDHVDILGPLSAQTGIAVLDMCASLKELLQNGMNGRTILGSALLE
DEFTPFDVVRQCSGVTFQSAVKRTIKGTHHWLLLTILTSLLVLVQSTQWSLFFFLYENAFLPFAMGI
IAMSAFAMMFVKHKHAFLCLFLLPSLATVAYFNMVYMPASWVMRIMTWLDMVDTSLSGFKLKDCVMY
ASAVVLLILMTARTVYDDGARRVWTLMNVLTLVYKVYYGNALDQAISMWALIISVTSNYSGVVTTVM
FLARGIVEMCVEYCPIFFITGNTLQCIMLVYCFLGYFCTCYFGLFCLLNRYFRLTLGVYDYLVSTQE
FRYMNSQGLLPPKNSIDAFKLNIKLLGVGGKPCIKVATVQSKMSDVKCTSVVLLSVLQQLRVESSSK
LWAQCVQLHNDILLAKDTTEAFEKMVSLLSVLLSMQGAVDINKLCEEMLDNRATLQAIASEFSSLPS
YAAFATAQEAYEQAVANGDSEVVLKKLKKSLNVAKSEFDRDAAMQRKLEKMADQAMTQMYKQARSED
KRAKVTSAMQTMLFTMLRKLDNDALNNIINNARDGCVPLNIIPLTTAAKLMVVIPDYNTYKNTCDGT
TFTYASALWEIQQVVDADSKIVQLSEISMDNSPNLAWPLIVTALRANSAVKLQNNELSPVALRQMSC
AAGTTQTACTDDNALAYYNTTKGGRFVLALLSDLQDLKWARFPKSDGTGTIYTELEPPCRFVTDTPK
GPKVKYLYFIKGLNNLNRGMVLGSLAATVRLQAGNATEVPANSTVLSFCAFAVDAAKAYKDYLASGG
QPITNCVKMLCTHTGTGQAITVTPEANMDQESFGGASCCLYCRCHIDHPNPKGFCDLKGKYVQIPTT
CANDPVGFTLKNTVCTVCGMWKGYGCSCDQLREPMLQSADAQSFLNRVCGVSAARLTPCGTGTSTDV
VYRAFDIYNDKVAGFAKFLKTNCCRFQEKDEDDNLIDSYFVVKRHTFSNYQHEETIYNLLKDCPAVA
KHDFFKFRIDGDMVPHISRQRLTKYTMADLVYALRHFDEGNCDTLKEILVTYNCCDDDYENKKDWYD
FVENPDILRVYANLGERVRQALLKTVQFCDAMRNAGIVGVLTLDNQDLNGNWYDFGDFIQTTPGSGV
PVVDSYYSLLMPILTLTRALTAESHVDTDLTKPYIKWDLLKYDFTEERLKLFDRYFKYWDQTYHPNC
VNCLDDRCILHCANFNVLFSTVFPPTSFGPLVRKIFVDGVPFVVSTGYHFRELGVVHNQDVNLHSSR
LSFKELLVYAADPAMHAASGNLLLDKRTTCFSVAALTNNVAFQTVKPGNENKDFYDFAVSKGFFKEG
SSVELKHFFFAQDGNAAISDYDYYRYNLPTMCDIRQLLFVVEVVDKYFDCYDGGCINANQVIVNNLD
KSAGFPFNKWGKARLYYDSMSYEDQDALFAYTKRNVIPTITQMNLKYAISAKNRARTVAGVSICSTM
TNRQFHQKLLKSIAATRGATVVIGTSKFYGGWHNMLKTVYSDVENPHLMGWDYPKCDRAMPNMLRIM
ASLVLARKHTTCCSLSHRFYRLANECAQVLSEMVMCGGSLYVKPGGTSSGDATTAYANSVENICQAV
TANVNALLSTDGNKIADKYVRNLQHRLYECLYRNRDVDTDFVNEFYAYLRKHFSMMILSDDAVVCEN
STYASQGLVASIKNFKSVLYYQNNVFMSEAKCWTETDLTKGPHEFCSQHTMLVKQGDDYVYLPYPDP
SRILGAGCFVDDIVKTDGTLMIERFVSLAIDAYPLTKHPNQEYADVFHLYLQYIRKLHDELTGHMLD
MYSVMLTNDNTSRYWEPEFYEAMYTPHTVLQAVGACVLCNSQTSLRCGACIRRPFLCCKCCYDHVIS
TSHKLVLSVNPYVCNAPGCDVTDVTQLYLGGMSYYCKSHKPPISFPLCANGQVFGLYKNTCVGSDNV
TDFNAIATCDWTNAGDYILANTCTERLKLFAAETLKATEETFKLSYGIATVREVLSDRELHLSWEVG
KPRPPLNRNYVFTGYRVTKNSKVQIGEYTFEKGDYGDAVVYRGTTTYKLNVGDYFVLTSHTVMPLSA
PTLVPQEHYVRITGLYPTLNISDEFSSNVANYQKVGMQKYSTLQGPPGTGKSHFAIGLALYYPSARI
VYTACSHAAVDALCEKALKYLPIDKCSRIIPARARVECFDKFKVNSTLEQYVFCTVNALPETTADIV
VEDEISMATNYDLSVVNARLRAKHYVYIGDPAQLPAPRTLLTKGTLEPEYFNSVCRLMKTIGPDMEL
GTCRRCPAEIVDTVSALVYDNKLKAHKDKSAQCFKMFYKGVITHDVSSAINRPQIGVVREFLTRNPA
WRKAVFISPYNSQNAVASKILGLPTQTVDSSQGSEYDYVIFTQTTETAHSCNVNRENVAITRAKVGI
LCIMSDRDLYDKLQFTSLEIPRRNVATLQAENVTGLFKDCSKVITGLHPTQAPTHLSVDTKFKTEGL
CVDIPGIPKDMTYRRLISMMGFKMNYQVNGYPNMFITREEAIRHVRAWIGFDVEGCHATREAVGTNL
PLQLGFSTGVNLVAVPTGYVDTPNNTDFSRVSAKPPPGDQFKHLIPLMYKGLPWNVVRIKIVQMLSD
TLKNLSDRVVFVLWAHGFELTSMKYFVKIGPERTCCLCDRRATCFSTASDTYACWHHSIGFDYVYNP
FMIDVQQWGFTGNLQSNHDLYCQVHGNAHVASCDAIMTRCLAVHECFVKRVDWTIEYPIIGDELKIN
AACRKVQHMVVKAALLADKFPVLHDIGNPKAIKCVPQADVEWKFYDAQPCSDKAYKIEELFYSYATH
SDKFTDGVCLFWNCNVDRYPANSIVCRFDTRVLSNLNLPGCDGGSLYVNKHAFHTPAFDKSAFVNLK
QLPFFYYSDSPCESHGKQVVSDIDYVPLKSATCITRQNLGGAVCRHHANEYRLYLDAYNMMISAGFS
LWVYKQFDTYNLWNTFTRLQSLENVAFNVVNKGHFDGQQGEVPVSIINNTVYTKVDGVDVELFENKT
TLPVNVAFELWAKRNIKPVPEVKILNNLGVDIAANTVIWDYKRDAPAHISTIGVCSMTDIAKKPTET
ICAPLTVFFDGRVDGQVDLFRNARNGVLITEGSVKGLQPSVGPKQASLNGVTLIGEAVKTQFNYYKK
VDGVVQQLPETYFTQSRNLQEFKPRSQMEIDFLELAMDEFIERYKLEGYAFEHIVYGDFSHSQLGGL
HLLIGLAKRFKESPFELEDFIPMDSTVKNYFITDAQTGSSKCVCSVIDLLLDDFVEIIKSQDLSVVS
KVVKVTIDYTEISFMLWCKDGHVETFYPKLQSSQAWQPGVAMPNLYKMQRMLLEKCDLQNYGDSATL
PKGIMMNVAKYTQLCQYLNTLTLAVPYNMRVIHFGAGSDKGVAPGTAVLRQWLPTGTLLVDSDLNDE
VSDADSTLIGDCATVHTANKWDLIISDMYDPKTKNVTKENDSKEGFFTYICGFIQQKLALGGSVAIK
ITEHSWNADLYKLMGHFAWWTAFVTNVNASSSEAFLIGCNYLGKPREQIDGYVMHANYIFWRNTNPI
QLSSYSLFDMSKFPLKLRGTAVMSLKEGQINDMILSLLSKGRLIIRENNRVVISSDVLVNN

In some embodiments, the antibodies described herein can be useful against SARS-CoV2 variants. For example, the variants can be: the UK variant B.1.1.7 (such as B.1.1.7 with S:E484K); the South African variant B.1.351; the California variant B.1.427; the California variant B.1.429; the Brazilian variant P.1; the Brazilian variant P.2; the New York variant B.1.526 (such as B.1.526 with S:E484K or B.1.526 with S:S477N); the New York variant B.1.526.1; the New York variant B.1.526.2, the amino acid mutations of each strain which can be accessed at https://outbreak.info/situation-reports#Lineage_Mutation, and is incorporated by reference in their entireties. For example, a variant of SARS-CoV2 has accession number YP_009724390.1. For example, a variant of SARS-CoV2 has accession number QHD43416.1.

The SARS-CoV2 variants can comprise, for instance, amino acid sequences having an identity to SEQ ID NO: 980 of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

Antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, IgG₃, IgG₄. Furthermore, in humans, the light chain can be a kappa chain or a lambda chain. The term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Minor variations in the amino acid sequences of proteins are provided by the antibodies described hereon. The variations in the amino acid sequence can be when the sequence maintains at least about 30%, at least about 40%, at least about 50%, at least about 60%, at least about 70%, at least about 75%, at least about 80%, at least about 90%, at least about 95%, or at least about 99% amino acid identity to the SEQ ID NOS of the antibodies described herein. For example, conservative amino acid replacements can be utilized. Conservative replacements are those that take place within a family of amino acids that are related in their side chains, wherein the interchangeability of residues have similar side chains In certain embodiments, the antibodies described herein include variants. Such variants can include those having at least from about 46% to about 50% amino acid identity to the SEQ ID NOS of the antibodies described herein, or having at least from about 50.1% to about 55% amino acid identity to the SEQ ID NOS of the antibodies described herein, or having at least from about 55.10% to about 60% amino acid identity to the SEQ ID NOS of the antibodies described herein, or having from at least about 60.10% to about 65% amino acid identity to the SEQ ID NOS of the antibodies described herein, or having from about 65.10% to about 70% amino acid identity to the SEQ ID NOS of the antibodies described herein, or having at least from about 70.10% to about 75% amino acid identity to the SEQ ID NOS of the antibodies described herein, or having at least from about 75.10% to about 80% amino acid identity to the SEQ ID NOS of the antibodies described herein, or having at least from about 80.1% to about 85% amino acid identity to the SEQ ID NOS of the antibodies described herein, or having at least from about 85.10% to about 90% amino acid identity to the SEQ ID NOS of the antibodies described herein, or having at least from about 90.1% to about 95% amino acid identity to the SEQ ID NOS of the antibodies described herein, or having at least from about 95.1% to about 97% amino acid identity to the SEQ ID NOS of the antibodies described herein, or having at least from about 97.1% to about 99% amino acid identity to the SEQ ID NOS of the antibodies described herein.

The term “epitope” can include any protein determinant that can specifically bind to an immunoglobulin, a scFv, or a T-cell receptor. Epitopic determinants can consist of chemically active surface groupings of molecules such as amino acids or sugar side chains and have specific three-dimensional structural characteristics, as well as specific charge characteristics. For example, antibodies can be raised against N-terminal or C-terminal peptides of a polypeptide, for example the C terminal domain (CTD) of the spike protein SARS-CoV2. The spike protein of SARS-CoV2 has NCBI Reference Sequence:

YP_009724390 (amino acid residues 1-1273; SEQ ID NO: 980) comprising sequence:
MFVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQPFLMDLEGKQ
GNFKNLREFVFKNIDGYFKIYSKHTPINLVRDLPQGFSALEPLVDLPIGINITRFQTLLAL
HRSYLTPGDSSSGWTAGAAAYYVGYLQPRTELLKYNENGTITDAVDCALDPLSETKCTLKS
FTVEKGIYQTSNFRVQPTESIVRFPNITNLCPFGEVENATRFASVYAWNRKRISNCVADYS

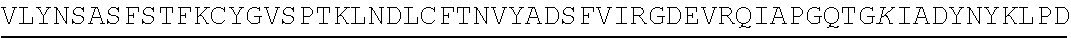

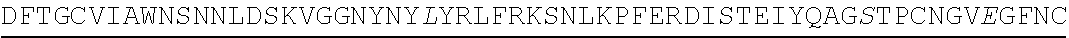

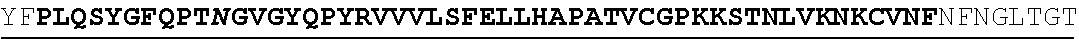

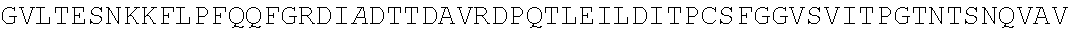

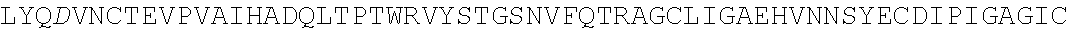

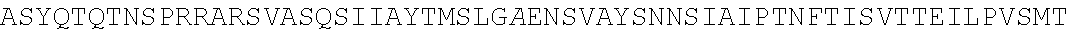

KTSVDCTMYICGDSTECSNLLLQYGSFCTQLNRALTGIAVEQDKNTQEVFAQVKQIYKTPP
IKDFGGFNFSQILPDPSKPSKRSFIEDLLENKVTLADAGFIKQYGDCLGDIAARDLICAQK
FNGLTVLPPLLTDEMIAQYTSALLAGTITSGWTFGAGAALQIPFAMQMAYRENGIGVTQNV
LYENQKLIANQFNSAIGKIQDSLSSTASALGKLQDVVNQNAQALNTLVKQLSSNFGAISSV
LNDILSRLDKVEAEVQIDRLITGRLQSLQTYVTQQLIRAAEIRASANLAATKMSECVLGOS
KRVDFCGKGYHLMSFPQSAPHGVVFLHVTYVPAQEKNFTTAPAICHDGKAHFPREGVFVSN
GTHWFVTQRNFYEPQIITTDNTFVSGNCDVVIGIVNNTVYDPLQPELDSFKEELDKYFKNH
TSPDVDLGDISGINASVVNIQKEIDRLNEVAKNLNESLIDLQELGKYEQYIKWPWYIWLGF
IAGLIAIVMVTIMLCCMTSCCSCLKGCCSCGSCCKFDEDDSEPVLKGVKLHYT

In some embodiments, the epitope comprises a region within amino acids 319-490 of the spike protein of SARS-CoV2 having NCBI Reference Sequence YP_009724390 (underlined). In some embodiments, the epitope comprises a region within amino acids 319-541 of the spike protein of SARS-CoV2 having NCBI Reference Sequence YP_009724390 (underlined and bolded). The exemplary, italicized shadowed amino acid residues of SEQ ID NO: 980 correspond to amino acid mutations found in SARS-CoV2 variant strains (e.g., K417N or K417T, L452R, S477N, E484K, N501Y, A570D, D614G, A701V).

The terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ allows the cancellation of all parameters not related to affinity, and is equal to the dissociation constant K_{d}. (See, generally, Davies et al. (1990) Annual Rev Biochem 59:439-473). An antibody of the invention can specifically bind to a SARS-CoV2 epitope when the equilibrium binding constant (K_{D}) is <1 μM, <10 μM, <10 nM, <10 μM, or <100 μM to about 1 μM, as measured by assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the K_{D} is about 6E-11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it can bind to a random, unrelated epitope.

For example, the SARS-CoV2 antibody can be monovalent or bivalent, and comprises a single or double chain. Functionally, the binding affinity of the SARS-CoV2 antibody is within the range of 10⁻⁵M to 10⁻¹² M. For example, the binding affinity of the SARS-CoV2 antibody is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰M, from 10⁻⁶ M to 10⁻¹⁰ M, from 10⁻⁷ M to 10⁻¹⁰ M, from 10⁻⁸ M to 10⁻¹⁰M, from 10⁻⁹ M to 10⁻¹⁰ M, from 10⁻⁵ M to 10⁻⁹ M, from 10⁻⁶ M to 10⁻⁹M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M, or from 10⁻⁵ M to 10⁻⁶ M.

A SARS-CoV2 protein or a derivative, fragment, analog, homolog or ortholog thereof, can be utilized as an immunogen in the generation of antibodies that immunospecifically bind these protein components.

Those skilled in the art will recognize that it is possible to determine, without undue experimentation, if a human monoclonal antibody has the same specificity as a human monoclonal antibody of the invention by ascertaining whether the former prevents the latter from binding to SARS-CoV2. If the human monoclonal antibody being tested competes with the human monoclonal antibody of the invention, as shown by a decrease in binding by the human monoclonal antibody of the invention, then the two monoclonal antibodies bind to the same, or to a closely related, epitope.

Another way to determine whether a human monoclonal antibody has the specificity of a human monoclonal antibody of the invention is to pre-incubate the human monoclonal antibody of the invention with the SARS-CoV2 with which it is normally reactive, and then add the human monoclonal antibody being tested to determine if the human monoclonal antibody being tested is inhibited in its ability to bind SARS-CoV2. If the human monoclonal antibody being tested is inhibited then, it has the same, or functionally equivalent, epitopic specificity as the monoclonal antibody of the invention.

Various procedures known within the art can be used for the production of polyclonal or monoclonal antibodies directed against a protein of the invention, or against derivatives, fragments, analogs homologs or orthologs thereof (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “MAb” or “monoclonal antibody composition”, as used herein, can refer to a population of antibody molecules that contain only one molecular species of antibody molecule consisting of a unique light chain gene product and a unique heavy chain gene product. For example, the complementarity determining regions (CDRs) of the monoclonal antibody are identical in the molecules of the population. MAbs contain an antigen binding site that is immunoreactive with an epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is immunized with an immunizing agent to elicit lymphocytes that can produce antibodies that will specifically bind to the immunizing agent. Alternatively, the lymphocytes can be immunized in vitro.

The immunizing agent can include the protein antigen, a fragment thereof or a fusion protein thereof. For example, peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are transformed mammalian cells, such as myeloma cells of rodent, bovine and human origin. Rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas will include hypoxanthine, aminopterin, and thymidine (“HAT medium”), which substances prevent the growth of HGPRT-deficient cells.

Immortalized cell lines include those that fuse efficiently, support stable high level expression of antibody by the selected antibody-producing cells, and are sensitive to a medium such as HAT medium. Immortalized cell lines can also include murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. The binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that can bind specifically to genes encoding the heavy and light chains of murine antibodies). The hybridoma cells of the invention serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies are antibody molecules in which the entire sequence of both the light chain and the heavy chain, including the CDRs, arise from human genes. Such antibodies are termed “human antibodies”, or “fully human antibodies” herein. Human monoclonal antibodies can be prepared by using trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies can be utilized and can be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

“Humanized antibodies” can be antibodies from a non-human species (such as mouse), whose amino acid sequences (for example, in the CDR regions) have been modified to increase their similarity to antibody variants produced in humans. Antibodies can be humanized by methods known in the art, such as CDR-grafting. See also, Safdari et al., (2013) Biotechnol Genet Eng Rev.; 29:175-86. In addition, humanized antibodies can be produced in transgenic plants, as an an inexpensive production alternative to existing mammalian systems. For example, the transgenic plant can be atobacco plant, i.e., Nicotiana benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can be readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the invention further provides any cell or plant comprising a vector that encodes the antibody of the invention, or produces the antibody of the invention.

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 73 65-93 (1995).

Human antibodies can additionally be produced using transgenic nonhuman animals which are modified so as to produce fully human antibodies rather than the animal's endogenous antibodies in response to challenge by an antigen. (See PCT publication WO94/02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides the desired modifications is then obtained as progeny by crossbreeding intermediate transgenic animals containing fewer than the full complement of the modifications. The embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

Thus, using such a technique, therapeutically useful IgG, IgA, IgM and IgE antibodies can be produced. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Creative BioLabs (Shirley, NY) can be engaged to provide human antibodies directed against a selected antigen using technology similar to that described herein.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, are disclosed in PCT publication WO 99/53049.

The antibody can be expressed by a vector containing a DNA segment encoding the single chain antibody described herein.

These vectors can include liposomes, naked DNA, adjuvant-assisted DNA, gene gun, catheters, etc. Vectors can include chemical conjugates such as described in WO 93/64701, which has targeting moiety (e.g. a ligand to a cellular surface receptor), and a nucleic acid binding moiety (e.g. polylysine), viral vectors (e.g. a DNA or RNA viral vector), fusion proteins such as described in PCT/US 95/02140 (WO 95/22618) which is a fusion protein containing a target moiety (e.g. an antibody specific for a target cell) and a nucleic acid binding moiety (e.g. a protamine), plasmids, phage, etc. The vectors can be chromosomal, non-chromosomal or synthetic. Retroviral vectors can also be used, and include moloney murine leukemia viruses.

DNA viral vectors can also be used, and include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cell's cytoplasm. Avipox virus vectors result in only a short-term expression of the nucleic acid. Adenovirus vectors, adeno-associated virus vectors and herpes simplex virus (HSV) vectors are useful for introducing the nucleic acid into neural cells. The adenovirus vector results in a shorter-term expression (about 2 months) than adeno-associated virus (about 4 months), which in turn is shorter than HSV vectors. The vector chosen will depend upon the target cell and the condition being treated. The introduction can be by standard techniques, e.g. infection, transfection, transduction or transformation. Examples of modes of gene transfer include e.g., naked DNA, CaPO4 precipitation, DEAE dextran, electroporation, protoplast fusion, lipofection, cell microinjection, and viral vectors.

The vector can be employed to target essentially any target cell. For example, stereotaxic injection can be used to direct the vectors (e.g. adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and can be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways. For example, to detect the presence of SARS-CoV2 in a sample. The antibody can also be used to try to bind to and disrupt SARS-CoV2.

In an embodiment, the antibodies of the invention are full-length antibodies, containing an Fc region similar to wild-type Fc regions that bind to Fc receptors.

Heteroconjugate antibodies are also within the scope of the invention. Heteroconjugate antibodies are composed of two covalently joined antibodies. It is intended that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In embodiments, the antibody of the invention can be modified with respect to effector function, so as to enhance, e.g., the effectiveness of the antibody in neutralizing or preventing viral infection. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)). In one embodiment, the antibody of the invention has modifications of the Fc region, such that the Fc region does not bind to the Fc receptors. For example, the Fc receptor is Fcγ receptor. Antibodies with modification of the Fc region such that the Fc region does not bind to Fcγ, but still binds to neonatal Fc receptor are useful as described herein.

In certain embodiments, an antibody of the invention can comprise an Fc variant comprising an amino acid substitution which alters the antigen-independent effector functions of the antibody, specifically the circulating half-life of the antibody. Such antibodies exhibit increased or decreased binding to FcRn when compared to antibodies lacking these substitutions, therefore, have an increased or decreased half-life in serum, respectively. Fc variants with improved affinity for FcRn can have longer serum half-lives, and such molecules have useful applications in methods of treating mammals where long half-life of the administered antibody is required for uses described herien, e.g., to treat a chronic disease or disorder. In contrast, Fc variants with decreased FcRn binding affinity can have shorter halt-lives, and such molecules are also useful, for example, for administration to a mammal where a shortened circulation time can be advantageous, e.g., for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity can be required for uses described herein include those applications in which localization to the brain, kidney, and/or liver is required. In one embodiment, the Fc variant-containing antibodies can exhibit reduced transport across the epithelium of kidney glomeruli from the vasculature. In another embodiment, the Fc variant-containing antibodies can exhibit reduced transport across the blood brain barrier (BBB) from the brain, into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering).

In some embodiments, mutations are introduced to the constant regions of the mAb such that the antibody dependent cell-mediated cytotoxicity (ADCC) activity of the mAb is altered. For example, the mutation is a LALA mutation in the CH2 domain. In one embodiment, the antibody (e.g., a human mAb, or a bispecific Ab) contains mutations on one scFv unit of the heterodimeric mAb, which reduces the ADCC activity. In another embodiment, the mAb contains mutations on both chains of the heterodimeric mAb, which completely ablates the ADCC activity. For example, the mutations introduced into one or both scFv units of the mAb are LALA mutations in the CH2 domain. These mAbs with variable ADCC activity can be optimized such that the mAbs exhibits maximal selective killing towards cells that express one antigen that is recognized by the mAb, however exhibits minimal killing towards the second antigen that is recognized by the mAb.

In other embodiments, antibodies of the invention for use in the diagnostic and treatment methods described herein have a constant region, e.g., an IgG₁ or IgG4 heavy chain constant region, which can be altered to reduce or eliminate glycosylation. For example, an antibody of the invention can also comprise an Fc variant comprising an amino acid substitution which alters the glycosylation of the antibody. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In one embodiment, the antibody comprises an Fc variant with an amino acid substitution at amino acid position 228 or 299 (EU numbering). In other embodiments, the antibody comprises an IgG1 or IgG4 constant region comprising an S228P and a T299A mutation (EU numbering).

Exemplary amino acid substitutions which confer reduced or altered glycosylation are described in PCT Publication No, WO05/018572, which is incorporated by reference herein in its entirety. In some embodiments, the antibodies of the invention, or fragments thereof, are modified to eliminate glycosylation. Such antibodies, or fragments thereof, can be referred to as “agly” antibodies, or fragments thereof, (e.g. “agly” antibodies). While not wishing to be bound by theory “agly” antibodies, or fragments thereof, can have an improved safety and stability profile in vivo. Exemplary agly antibodies, or fragments thereof, comprise an aglycosylated Fc region of an IgG4 antibody which is devoid of Fc-effector function thereby eliminating the potential for Fc mediated toxicity to the normal vital tissues. In yet other embodiments, antibodies of the invention, or fragments thereof, comprise an altered glycan. For example, the antibody can have a reduced number of fucose residues on an N-glycan at Asn297 of the Fc region, i.e., is afucosylated. In another embodiment, the antibody can have an altered number of sialic acid residues on the N-glycan at Asn297 of the Fc region.

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The binding is, however, covalent binding. Covalent binding can be achieved by direct condensation of existing side chains or by the incorporation of external bridging molecules. Many bivalent or polyvalent linking agents are useful in coupling protein molecules, such as the antibodies of the invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987)). Examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Useful linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described herein contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, for example, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Non-limiting example of useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the invention can be conjugated to the liposomes as described in Martin et al., J. Biol. Chem., 257: 286-288 (1982) via a disulfide-interchange reaction.

The generation of neutralization escape mutants can be a helpful tool for identifying residues critical for neutralization and for investigating virus evolution under immune pressure (37, 49). Like other RNA viruses, CoVs have high mutation rates, especially during cross-species transmission, which is important for virus adaptation to new host receptors (5, 6, 50). Immune pressure is another force selecting virus mutation (37, 49). In addition, in view of current recommendations by the International Severe Acute Respiratory and Emerging Infection Consortium (ISARIC) and the increasing recognition that human Abs can have a role in the management of infectious diseases, the therapeutic potential of these nAbs can be considered for the prophylaxis and treatment of SARS (53). While escape from neutralization is a concern with therapeutic Abs, our study provides reagents and a strategy to mitigate this potential problem.

In another embodiment, the antibodies that neutralize infection by Severe Acute Respiratory Syndrome-associated coronavirus (SARS-CoV2) can be belong to various kinds of antibody classes and isotypes. For example, the neutralizing antibodies can be IgG₁, IgG₂, IgG₃ and/or IgG₄ isotype antibodies.

In another embodiment, the neutralizing antibodies can also contain LALA mutations in the Fc region. The LALA double mutants are characterized by the L234A L235)-A amino acid substitutions.

The humanized antibodies described herein can be produced in mammalian expression systems, such as hybridomas. The humanized antibodies described herein can also be produced by non-mammalian expression systems, for example, by transgenic plants. For example, the antibodies described herein are produced in transformed tobacco plants (N. benthamiana and N. tabaccum).

### Multispecific Antibodies

Multispecific antibodies are antibodies that can recognize two or more different antigens. For example, a bi-specific antibody (bsAb) is an antibody comprising two variable domains or scFv units such that the resulting antibody recognizes two different antigens. For example, a trispecific antibody (tsAb) is an antibody comprising two variable domains or scFv units such that the resulting antibody recognizes three different antigens. This invention provides for multispecific antibodies, such as bi-specific and trispecific antibodies, that recognize ACE-2 and/or a second antigen and/or a third antigen (for example, a SARS-CoV-2 target). Exemplary second or third antigens include the SARS-CoV2 spike (S) glycoprotein (e.g., comprising a S1 subunit (which further comprises a receptor binding domain, RBD, located in the S1 subunit) and S2 subunit in each spike monomer), small envelope (E) glycoprotein, the N-terminal domain (NTD), or the membrane (M) glycoprotein. In one embodiment, the antigen comprises amino acids 318-510 in the S1 domain of the SARS-CoV-2 Spike protein (e.g., the CR3022 epitope). In one embodiment, the antigen comprises amino acids 1-290 in the NTD of SARS-CoV-2. For example, a bispecific antibody can be developed that targets ACE2 and CR3022 epitopes to fix RBD in the “up” position, thus exposing the S2′ cleavage site and facilitating irreversible S protein transition into more stable postfusion conformation. For example, a trispecific antibody can be developed with a tandem scFv-Fc (e.g., an epitope specific for ACE2 and an epitope specific for CR3022) on one side, and a mono scFv-Fc on the other (e.g., an epitope specific for the NTD of SARS-CoV-2). Without wishing to be bound by theory, heterodimerization can lead to a trivalent antibody that targets 3 epitopes on the S1 domain of the spike. Without wishing to be bound by theory, multispecific antibodies (e.g., bispecific antibodies and trispecific antibodies) can be engineered that bind to distinct, non-overlapping epitopes on the S protein RBD. In one embodiment, multispecific antibodies (e.g., bi-specific antibodies and trispecific antibodies) can comprise SARS-CoV2 specific fusion proteins encompassing the antibodies described herein. For example, the fusion protein comprises an antibody comprising a variable domain or scFv unit and a second antigen and/or a third antigen described herein such that the resulting antibody recognizes said antigen and binds to it. In one embodiment, the fusion protein further comprises a constant region, and/or a linker as described herein. Different formats of multispecific antibodies (e.g., bispecific antibodies and trispecific antibodies) are also provided herein. In some embodiments, each of the anti-SARS-CoV2 fragment and the second antigen-specific fragment and/or the third antigen-specific fragment is each independently selected from a Fab fragment, a single-chain variable fragment (scFv), or a single-domain antibody. In some embodiments, the multispecific antibody (e.g., bispecific antibody and trispecific antibody) further includes a Fc fragment. Multispecific antibodies (e.g., bispecific antibodies and trispecific antibodies) of the invention comprise a heavy chain and a light chain combination or scFv of the SARS-CoV-2 antibodies disclosed herein.

Multispecific antibodies (e.g., bispecific antibodies and trispecific antibodies) of the invention (for example, an anti-SARS-CoV2-scFv fusion protein) can be constructed using methods known art. In some embodiments, the bi-specific antibody is a single polypeptide wherein the two scFv fragments are joined by a long linker polypeptide, of sufficient length to allow intramolecular association between the two scFv units to form an antibody. In other embodiments, the bi-specific antibody is more than one polypeptide linked by covalent or non-covalent bonds. In some embodiments, the amino acid linker (GGGGSGGGGS; “(G4S)2”) that can be used with anti-SARS-CoV2-scFv fusion constructs can be generated with a longer G4S linker to improve flexibility. For example, the linker can also be “(G4S)3” (e.g., GGGGSGGGGSGGGGS); “(G4S)4” (e.g., GGGGSGGGGSGGGGSGGGGS); “(G4S)5” (e.g., GGGGSGGGGSGGGGSGGGGSGGGGS); “(G4S)6” (e.g., GGGGSGGGGSGGGGSGGGGSGGGGSGGGGS); “(G4S)7” (e.g., GGGGSGGGGSGGGGSGGGGSGGGGSGGGGSGGGGS); and the like. For example, use of the (G4S)5 linker can provide more flexibility and can improve expression. In some embodiments, the linker can also be (GS)ₙ, (GGS)ₙ, (GGGS)ₙ, (GGSG)ₙ, (GGSGG)ₙ, or (GGGGS)ₙ, wherein n is 1, 2, 3, 4, 5, 6, 7, 8, 9, or 10. Non-limiting examples of linkers known to those skilled in the art that can be used to construct the anti-SARS-CoV2-scFv fusions described herein can be found in U.S. Pat. No. 9,708,412; U.S. Patent Application Publication Nos. US 20180134789 and US 20200148771; and PCT Publication No. WO2019051122 (each of which are incorporated by reference in their entireties).

In another embodiment, the multispecific antibodies (e.g., bispecific antibodies and trispecific antibodies such asanti-SARS-CoV2-scFv fusions) can be constructed using the “knob into hole” method (Ridgway et al, Protein Eng 7:617-621 (1996)). In this method, the Ig heavy chains of the two different variable domains are reduced to selectively break the heavy chain pairing while retaining the heavy-light chain pairing. The two heavy-light chain heterodimers that recognize two different antigens or three different antigens are mixed to promote heteroligation pairing, which can be mediated through the engineered “knob into holes” of the CH3 domains.

In another embodiment, multispecific antibodies (e.g., bispecific antibodies and trispecific antibodies such as anti-SARS-CoV2-scFv fusions) can be constructed through exchange of heavy-light chain dimers from two or more different antibodies to generate a hybrid antibody where the first heavy-light chain dimer recognizes a first antigen, such as ACE-2, and the second heavy-light chain dimer recognizes a second and or third antigen, such as the SARS-CoV2 spike (S) glycoprotein (e.g., comprising a S1 subunit (which further comprises a receptor binding domain, RBD, located in the S1 subunit) and S2 subunit in each spike monomer), small envelope (E) glycoprotein, NTD, or the membrane (M) glycoprotein. The mechanism for heavy-light chain dimer is similar to the formation of human IgG4, which can also function as a bispecific molecule. Dimerization of IgG heavy chains is driven by intramolecular force, such as pairing the CH3 domain of each heavy chain and disulfide bridges. Presence of a specific amino acid in the CH3 domain (R409) has been shown to promote dimer exchange and construction of the IgG4 molecules. Heavy chain pairing is also stabilized further by interheavy chain disulfide bridges in the hinge region of the antibody. Specifically, in IgG4, the hinge region contains the amino acid sequence Cys-Pro-Ser-Cys (in comparison to the stable IgG1 hinge region which contains the sequence Cys-Pro-Pro-Cys) at amino acids 226-230. This sequence difference of Serine at position 229 has been linked to the tendency of IgG4 to form intrachain disulfides in the hinge region (Van der Neut Kolfschoten, M. et al, 2007, Science 317: 1554-1557 and Labrijn, A. F. et al, 2011, Journal of Immunol 187:3238-3246).

Multispecific antibodies (e.g., bispecific antibodies and trispecific antibodies) of the invention can be created through introduction of the R409 residue in the CH3 domain and the Cys-Pro-Ser-Cys sequence in the hinge region of antibodies that recognize SARS-CoV2, ACE-2 or a second and/or third antigen, so that the heavy-light chain dimers exchange to produce an antibody molecule with one heavy-light chain dimer recognizing SARS-CoV2 and/or ACE2 and the second heavy-light chain dimer recognizing a second and/or third antigen, wherein the second antigen or third antigen is any antigen described herein. Known IgG4 molecules can also be altered such that the heavy and light chains recognize SARS-CoV2 and/or ACE2 or a second and/or third antigen, as described herein. Use of this method for constructing the multispecific antibodies (e.g., bispecific antibodies and trispecific antibodies) of the invention can be beneficial due to the intrinsic characteristic of IgG4 molecules wherein the Fc region differs from other IgG subtypes in that it interacts poorly with effector systems of the immune response, such as complement and Fc receptors expressed by certain white blood cells. This specific property makes these IgG4-based bi-specific antibodies attractive for therapeutic applications, in which the antibody is required to bind the target(s) and functionally alter the signaling pathways associated with the target(s), however not trigger effector activities.

The multispecific antibodies (e.g., bispecific antibodies and trispecific antibodies) described herein (for example, anti-SARS-CoV2-scFv fusions) can be engineered with a non-depleting heavy chain isotype, such as IgG1-LALA or stabilized IgG4 or one of the other non-depleting variants.

In some embodiments, mutations are introduced to the constant regions of the multispecific antibodies (e.g., bispecific antibodies and trispecific antibodies) such that the antibody dependent cell-mediated cytotoxicity (ADCC) activity of the bsAb or tsAb is altered. For example, the mutation is a LALA mutation in the CH2 domain. In one aspect, the multispecific antibody (e.g., bispecific antibody and trispecific antibody) contains mutations on one scFv unit of the heterodimeric multispecific antibody, which reduces the ADCC activity. In another aspect, the multispecific antibody (e.g., bispecific antibody and trispecific antibody) contains mutations on both chains of the heterodimeric multispecific antibody, which completely ablates the ADCC activity. For example, the mutations introduced one or both scFv units of the multispecific antibody (e.g., bispecific antibody and trispecific antibody) are LALA mutations in the CH2 domain. These multispecific antibodies (e.g., bispecific antibodies and trispecific antibodies) with variable ADCC activity can be optimized such that the multispecific antibodies exhibit maximal selective killing towards cells that express one antigen that is recognized by the multispecific antibody, however exhibits minimal killing towards the second and/or third antigen that is recognized by the multispecific antibody.

The multispecific antibodies (e.g., bispecific antibodies and trispecific antibodies) disclosed herein can be useful in treatment of chronic infections, diseases, or medical conditions associated with COVID-19.

### Use of Antibodies Against SARS-CoV2

Methods for the screening of antibodies that possess the desired specificity include, but are not limited to, enzyme linked immunosorbent assay (ELISA) and other immunologically mediated techniques known within the art.

Antibodies directed against a SARS-CoV2 protein disclosed herein can be useful in treatment of chronic infections, diseases, or medical conditions associated with COVID-19. Antibodies directed against a SARS-CoV2 protein, such as the spike protein, can be used in methods known within the art relating to the localization and/or quantitation of SARS-CoV2 (e.g., for use in measuring levels of the SARS-CoV2 protein within appropriate physiological samples, for use in diagnostic methods, for use in imaging the protein, and the like). In a given embodiment, antibodies specific to a SARS-CoV2, or derivative, fragment, analog or homolog thereof, that contain the antibody derived antigen binding domain, are utilized as pharmacologically active compounds (referred to hereinafter as “Therapeutics”).

An antibody specific for a SARS-CoV2 protein can be used to isolate a SARS-CoV2 polypeptide by standard techniques, such as immunoaffinity, chromatography or immunoprecipitation. Antibodies directed against a SARS-CoV2 protein (or a fragment thereof) can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies of the invention, including polyclonal, monoclonal, humanized and fully human antibodies, can be used as therapeutic agents. Such agents can be employed to treat or prevent a SARS-CoV2-related disease or pathology in a subject. An antibody preparation, for example, one having high specificity and high affinity for its target antigen, is administered to the subject and can have an effect due to its binding with the target. Administration of the antibody can abrogate or inhibit or interfere with the internalization of the virus into a cell. In this case, the antibody binds to the target and prevents SARS-CoV2 binding the ACE2 receptor.

A therapeutically effective amount of an antibody of the invention includes the amount needed to achieve a therapeutic objective. As noted herein, this can be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention can be, by way of nonlimiting example, from about 0.1 mg/kg body weight to about 50 mg/kg body weight. Common dosing frequencies can range, for example, from twice daily to once a week.

Antibodies specifically binding a SARS-CoV2 protein or a fragment thereof of the invention, as well as other molecules identified by the screening assays disclosed herein, can be administered for the treatment of SARS-CoV2-related disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa., 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Embodiments of the invention can comprise antibody fragments, such as antibody fragments lacking an Fc region. Peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compound as necessary for the indication being treated, such as those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid allows for release of molecules for over 100 days, certain hydrogels release proteins for shorter time periods.

An antibody according to the invention can be used as an agent for detecting the presence of a SARS-CoV2 (or a protein or a protein fragment thereof) in a sample. In embodiments, the antibody contains a detectable label. Antibodies can be polyclonal, or for example, monoclonal. In embodiments, the antibody is an intact antibody. The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

### Chimeric Antigen Receptor (CAR) T-Cell Therapies

Cellular therapies, such as chimeric antigen receptor (CAR) T-cell therapies, are also provided herein. CAR T-cell therapies redirect a patient's T-cells to kill tumor cells by the exogenous expression of a CAR on a T-cell, for example. A CAR can be a membrane spanning fusion protein that links the antigen recognition domain of an antibody to the intracellular signaling domains of the T-cell receptor and co-receptor. A suitable cell can be used, for example, that can secrete an anti-SARS-CoV2 antibody of the invention (or alternatively engineered to express an anti-SARS-CoV2 antibody as described herein to be secreted). The anti-SARS-CoV2 “payloads” to be secreted, can be, for example, minibodies, scFvs, IgG molecules, bispecific fusion molecules, and other antibody fragments as described herein. Upon contact or engineering, the cell described herein can then be introduced to a patient in need of a treatment by infusion therapies known to one of skill in the art. The patient can have a SARS-CoV2 disease, such as COVID-19. The cell (e.g., a T cell) can be, for instance, T lymphocyte, a CD4+ T cell, a CD8+ T cell, or the combination thereof, without limitation. Exemplary CARs and CAR factories useful in aspects of the invention include those disclosed in, for example, PCT/US2015/067225 and PCT/US2019/022272, each of which are hereby incorporated by reference in their entireties. In one embodiment, the SARS-CoV2 antibodies discussed herein can be used in the construction of multi-specific antibodies or as the payload for a CAR-T cell. For example, in one embodiment, the anti-SARS-CoV2 antibodies discussed herein can be used for the targeting of the CARS (i.e., as the targeting moiety). In another embodiment, the anti-SARS-CoV2 antibodies discussed herein can be used as the targeting moiety, and a different SARS-CoV2 antibody that targets a different epitope can be used as the payload. In another embodiment, the payload can be an immunomodulatory antibody payload.

### Pharmaceutical Compositions

The antibodies or agents of the invention (also referred to herein as “active compounds”), and derivatives, fragments, analogs and homologs thereof, can be incorporated into pharmaceutical compositions suitable for administration. Such compositions comprise the antibody or agent and a pharmaceutically acceptable carrier. As used herein, the term “pharmaceutically acceptable carrier” can include solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Non-limiting examples of such carriers or diluents include, but are not limited to, water, saline, ringer's solutions, dextrose solution, and 5% human serum albumin. Liposomes and non-aqueous vehicles such as fixed oils can also be used. The use of such media and agents for pharmaceutically active substances is well known in the art. Except insofar as any conventional media or agent is incompatible with the active compound, use thereof in the compositions is intended. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The composition must be sterile and can be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be useful to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated herein, as required, followed by filtered sterilization. Dispersions can be prepared by incorporating the active compound into a sterile vehicle that contains a basic dispersion medium and the required other ingredients from those enumerated herein. In the case of sterile powders for the preparation of sterile injectable solutions, methods of preparation are vacuum drying and freeze-drying that yields a powder of the active ingredient plus any additional ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In embodiments, compositions, such as oral or parenteral compositions, can be formulated in dosage unit form for ease of administration and uniformity of dosage. Dosage unit form as used herein refers to physically discrete units suited as unitary dosages for the subject to be treated; each unit containing a predetermined quantity of active compound calculated to produce the therapeutic effect in association with the required pharmaceutical carrier. The specification for the dosage unit forms of the invention are dictated by and directly dependent on the unique characteristics of the active compound and the therapeutic effect to be achieved, and the limitations inherent in the art of compounding such an active compound for the treatment of individuals.

The pharmaceutical compositions can be included in a container, pack, or dispenser together with instructions for administration.

### Screening Methods

The invention provides methods (also referred to herein as “screening assays”) for identifying modulators, i.e., candidate or test compounds or agents (e.g., peptides, peptidomimetics, small molecules or other drugs) that modulate or otherwise interfere with the fusion of a SARS-CoV2 to the cell membrane. Also provided are methods of identifying compounds useful to treat SARS-CoV2 infection. The invention also encompasses compounds identified using the screening assays described herein.

For example, the invention provides assays for screening candidate or test compounds which modulate the interaction between the SARS-CoV2 and the cell membrane. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. (See, e.g., Lam, 1997. Anticancer Drug Design 12: 145).

A “small molecule” as used herein, can refer to a composition that has a molecular weight of less than about 5 kD, for example less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds can be presented in solution (see e.g., Houghten, 1992, Biotechniques 13: 412-421), or on beads (see Lam, 1991. Nature 354: 82-84), on chips (see Fodor, 1993, Nature 364: 555-556), bacteria (see U.S. Pat. No. 5,223,409), spores (see U.S. Pat. No. 5,233,409), plasmids (see Cull, et al., 1992, Proc. Natl. Acad. Sci. USA 89: 1865-1869) or on phage (see Scott and Smith, 1990, Science 249: 386-390; Devlin, 1990, Science 249: 404-406; Cwirla, et al., 1990, Proc. Natl. Acad. Sci. U.S.A. 87: 6378-6382; Felici, 1991, J. Mol. Biol. 222: 301-310; and U.S. Pat. No. 5,233,409.).

In one embodiment, a candidate compound is introduced to an antibody-antigen complex and determining whether the candidate compound disrupts the antibody-antigen complex, wherein a disruption of this complex indicates that the candidate compound modulates the interaction between a SARS-CoV2 and the cell membrane.

In another embodiment, at least one SARS-CoV2 protein is provided, which is exposed to at least one neutralizing monoclonal antibody. Formation of an antibody-antigen complex is detected, and one or more candidate compounds are introduced to the complex. If the antibody-antigen complex is disrupted following introduction of the one or more candidate compounds, the candidate compounds is useful to treat a SARS-CoV2-related disease or disorder. For example, the at least one SARS-CoV2 protein can be provided as a SARS-CoV2 molecule.

Determining the ability of the test compound to interfere with or disrupt the antibody-antigen complex can be accomplished, for example, by coupling the test compound with a radioisotope or enzymatic label such that binding of the test compound to the antigen or biologically-active portion thereof can be determined by detecting the labeled compound in a complex. For example, test compounds can be labeled with ¹²⁵I, ³⁵S, ¹⁴C, or ³H, directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product.

In one embodiment, the assay comprises contacting an antibody-antigen complex with a test compound, and determining the ability of the test compound to interact with the antigen or otherwise disrupt the existing antibody-antigen complex. In this embodiment, determining the ability of the test compound to interact with the antigen and/or disrupt the antibody-antigen complex comprises determining the ability of the test compound to bind to the antigen or a biologically-active portion thereof, as compared to the antibody.

In another embodiment, the assay comprises contacting an antibody-antigen complex with a test compound and determining the ability of the test compound to modulate the antibody-antigen complex. Determining the ability of the test compound to modulate the antibody-antigen complex can be accomplished, for example, by determining the ability of the antigen to bind to or interact with the antibody, in the presence of the test compound.

Those skilled in the art will recognize that, in any of the screening methods disclosed herein, the antibody can be a SARS-CoV2 neutralizing antibody or any variant thereof wherein the Fc region is modified such that it has reduced binding or does not bind to the Fc-gamma receptor. Additionally, the antigen can be a SARS-CoV2 protein, or a portion thereof.

The screening methods disclosed herein can be performed as a cell-based assay or as a cell-free assay. The cell-free assays of the invention are amenable to use of both the soluble form or the membrane-bound form of the proteins and fragments thereof. In the case of cell-free assays comprising the membrane-bound forms of the proteins, it can be desirable to utilize a solubilizing agent such that the membrane-bound form of the proteins are maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment, it can be desirable to immobilize the antibody or the antigen to facilitate separation of complexed from uncomplexed forms of one or both following introduction of the candidate compound, as well as to accommodate automation of the assay. Observation of the antibody-antigen complex in the presence and absence of a candidate compound can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-antibody fusion proteins or GST-antigen fusion proteins can be adsorbed onto glutathione Sepharose beads (Sigma Chemical, St. Louis, MO) or glutathione derivatized microtiter plates, that are then combined with the test compound, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined directly or indirectly. Alternatively, the complexes can be dissociated from the matrix, and the level of antibody-antigen complex formation can be determined using standard techniques.

Other techniques for immobilizing proteins on matrices can also be used in the screening assays of the invention. For example, the antibody or the antigen can be immobilized utilizing conjugation of biotin and streptavidin. Biotinylated antibody or antigen molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, other antibodies reactive with the antibody or antigen of interest, but which do not interfere with the formation of the antibody-antigen complex of interest, can be derivatized to the wells of the plate, and unbound antibody or antigen trapped in the wells by antibody conjugation. Methods for detecting such complexes, in addition to those described herein for the GST-immobilized complexes, include immunodetection of complexes using such other antibodies reactive with the antibody or antigen.

The invention further pertains to new agents identified by any of the aforementioned screening assays and uses thereof for treatments as described herein.

### Diagnostic Assays

Antibodies of the invention can be detected by or used for detection purposes by appropriate assays, e.g., conventional types of immunoassays such as sandwich ELISAs. For example, an assay can be performed in which a SARS-CoV2 or fragment thereof is affixed to a solid phase. Incubation is maintained for a sufficient period of time to allow the antibody in the sample to bind to the immobilized polypeptide on the solid phase. After this first incubation, the solid phase is separated from the sample. The solid phase is washed to remove unbound materials and interfering substances such as non-specific proteins which can also be present in the sample. The solid phase containing the antibody of interest bound to the immobilized polypeptide is subsequently incubated with a second, labeled antibody or antibody bound to a coupling agent such as biotin or avidin. This second antibody can be another anti-SARS-CoV2 antibody or another antibody. Labels for antibodies are well-known in the art and include radionuclides, enzymes (e.g. maleate dehydrogenase, horseradish peroxidase, glucose oxidase, catalase), fluors (fluorescein isothiocyanate, rhodamine, phycocyanin, fluorescarmine), biotin, and the like. The labeled antibodies are incubated with the solid and the label bound to the solid phase is measured. These and other immunoassays can be easily performed by those of ordinary skill in the art.

An exemplary method for detecting the presence or absence of a SARS-CoV2 in a biological sample involves obtaining a biological sample from a test subject and contacting the biological sample with a labeled monoclonal antibody according to the invention such that the presence of the SARS-CoV2 is detected in the biological sample.

As used herein, the term “labeled”, with regard to the probe or antibody, can refer to direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can refer to tissues, cells and biological fluids isolated from a subject, as well as tissues, cells and fluids present within a subject. That is, the detection method of the invention can be used to detect a SARS-CoV2 in a biological sample in vitro as well as in vivo. For example, in vitro techniques for detection of a SARS-CoV2 include enzyme linked immunosorbent assays (ELISAs), Western blots, immunoprecipitations, and immunofluorescence. Furthermore, in vivo techniques for detection of a SARS-CoV2 include introducing into a subject a labeled anti-SARS-CoV2 antibody. For example, the antibody can be labeled with a radioactive marker whose presence and location in a subject can be detected by standard imaging techniques.

In one embodiment, the biological sample contains protein molecules from the test subject. For example, one biological sample is a peripheral blood leukocyte sample isolated by conventional means from a subject.

The invention also encompasses kits for detecting the presence of a SARS-CoV2 in a biological sample. For example, the kit can comprise: a labeled compound or agent that can detect a SARS-CoV2 (e.g., an anti-SARS-CoV2 monoclonal antibody) in a biological sample; means for determining the amount of a SARS-CoV2 in the sample; and means for comparing the amount of a SARS-CoV2 in the sample with a standard. The compound or agent can be packaged in a suitable container. The kit can further comprise instructions for using the kit to detect a SARS-CoV2 in a sample.

### Passive Immunization

Passive immunization has proven to be an effective and safe strategy for the prevention and treatment of viral diseases. (See Keller et al., Clin. Microbiol. Rev. 13:602-14 (2000); Casadevall, Nat. Biotechnol. 20:114 (2002); Shibata et al., Nat. Med. 5:204-10 (1999); and Igarashi et al., Nat. Med. 5:211-16 (1999), each of which are incorporated herein by reference)). Passive immunization using neutralizing human monoclonal antibodies can provide an immediate treatment strategy for emergency prophylaxis and treatment of SARS-CoV2 infection and related diseases and disorders while the alternative and more time-consuming development of vaccines and new drugs in underway.

Subunit vaccines potentially offer significant advantages over conventional immunogens. They avoid the safety hazards inherent in production, distribution, and delivery of conventional killed or attenuated whole-pathogen vaccines. Furthermore, they can be rationally designed to include only confirmed protective epitopes, thereby avoiding suppressive T epitopes (see Steward et al., J. Virol. 69:7668 (1995)) or immunodominant B epitopes that subvert the immune system by inducing futile, non-protective responses (e.g. “decoy” epitopes). (See Garrity et al., J. Immunol. 159:279 (1997)).

Moreover, those skilled in the art will recognize that good correlation exists between the antibody neutralizing activity in vitro and the protection in vivo for many different viruses, challenge routes, and animal models. (See Burton, Natl. Rev. Immunol. 2:706-13 (2002); Parren et al., Adv. Immunol. 77:195-262 (2001)).

### Antigen-Ig Chimeras in Vaccination

It has been over a decade since the first antibodies were used as scaffolds for the efficient presentation of antigenic determinants to the immune systems. (See Zanetti, Nature 355:476-77 (1992); Zaghouani et al., Proc. Natl. Acad. Sci. USA 92:631-35 (1995)). When a peptide is included as an integral part of an IgG molecule (e.g., the 11 A or 256 IgG1 monoclonal antibody described herein), the antigenicity and immunogenicity of the peptide epitopes are greatly enhanced as compared to the free peptide. Such enhancement can be due to the antigen-IgG chimeras longer half-life, better presentation and constrained conformation, which mimic their native structures.

Moreover, an added advantage of using an antigen-Ig chimera is that the variable or the Fc region of the antigen-Ig chimera can be used for targeting professional antigen-presenting cells (APCs). To date, recombinant Igs have been generated in which the complementarity-determining regions (CDRs) of the heavy chain variable gene (V_{H}) are replaced with various antigenic peptides recognized by B or T cells. Such antigen-Ig chimeras have been used to induce both humoral and cellular immune responses. (See Bona et al., Immunol. Today 19:126-33 (1998)).

For example, chimeras with specific epitopes engrafted into the CDR3 loop have been used to induce humoral responses to HIV-1 gp120 V3-loop or the first extracellular domain (D1) of human CD4 receptor. (See Lanza et al., Proc. Natl. Acad. Sci. USA 90:11683-87 (1993); Zaghouani et al., Proc. Natl. Acad. Sci. USA 92:631-35 (1995)). The immune sera were able to prevent infection of CD4 SupT1 cells by HIV-1MN (anti-gp120 V3C) or inhibit syncytia formation (anti-CD4-D1). The CDR2 and CDR3 can be replaced with peptide epitopes simultaneously, and the length of peptide inserted can be up to 19 amino acids long.

Alternatively, one group has developed a “troybody” strategy in which peptide antigens are presented in the loops of the Ig constant (C) region and the variable region of the chimera can be used to target IgD on the surface of B-cells or MHC class II molecules on professional APCs including B-cells, dendritic cells (DC) and macrophages. (See Lunde et al., Biochem. Soc. Trans. 30:500-6 (2002)).

An antigen-Ig chimera can also be made by directly fusing the antigen with the Fc portion of an IgG molecule. You et al., Cancer Res. 61:3704-11 (2001) were able to obtain the arms of specific immune response, including very high levels of antibodies to hepatitis B virus core antigen using this method.

### DNA Vaccination

DNA vaccines are stable, can provide the antigen an opportunity to be naturally processed, and can induce a longer-lasting response. Although a very attractive immunization strategy, DNA vaccines often have very limited potency to induce immune responses. Poor uptake of injected DNA by professional APCs, such as dendritic cells (DCs), can be the main cause of such limitation. Combined with the antigen-Ig chimera vaccines, a promising new DNA vaccine strategy based on the enhancement of APC antigen presentation has been reported (see Casares, et al., Viral Immunol. 10:129-36 (1997); Gerloni et al., Nat. Biotech. 15:876-81 (1997); Gerloni et al., DNA Cell Biol. 16:611-25 (1997); You et al., Cancer Res. 61:3704-11 (2001)), which takes advantage of the presence of Fc receptors (FcγRs) on the surface of DCs.

An embodiment comprises a DNA vaccine encoding an antigen (Ag)-Ig chimera. Upon immunization, Ag-Ig fusion proteins will be expressed and secreted by the cells taking up the DNA molecules. The secreted Ag-Ig fusion proteins, while inducing B-cell responses, can be captured and internalized by interaction of the Fc fragment with FcγRs on DC surface, which will promote efficient antigen presentation and greatly enhance antigen-specific immune responses. Applying the same principle, DNA encoding antigen-Ig chimeras carrying a functional anti-MHC II specific scFv region gene can also target the immunogens to the three types of APCs. The immune responses can be further boosted with use of the same protein antigens generated in vitro (i.e., “prime and boost”), if necessary. Using this strategy, specific cellular and humoral immune responses against infection of SARS-CoV2 were accomplished through intramuscular (i.m.) injection of a DNA vaccine. (See Casares et al., Viral. Immunol. 10:129-36 (1997)).

### Vaccine Compositions

Therapeutic or prophylactic compositions are provided herein, which comprise mixtures of one or more monoclonal antibodies or ScFvs and combinations thereof. The prophylactic vaccines can be used to prevent a SARS-CoV2 infection and the therapeutic vaccines can be used to treat individuals following a SARS-CoV2 infection. Prophylactic uses include the provision of increased antibody titer to a SARS-CoV2 in a vaccination subject. In this manner, subjects at high risk of contracting SARS-CoV2 can be provided with passive immunity to a SARS-CoV2.

These vaccine compositions can be administered in conjunction with ancillary immunoregulatory agents. For example, cytokines, lymphokines, and chemokines, including, but not limited to, IL-2, modified IL-2 (Cys125→Ser125), GM-CSF, IL-12, γ-interferon, IP-10, MIP1β, and RANTES.

### Methods of Immunization

The vaccines of the invention have superior immunoprotective and immunotherapeutic properties over other anti-viral vaccines.

The invention provides a method of immunization, e.g., inducing an immune response, of a subject. A subject is immunized by administration to the subject a composition containing a membrane fusion protein of a pathogenic spike protein. The fusion protein is coated or embedded in a biologically compatible matrix.

The fusion protein is glycosylated, e.g. contains a carbohydrate moiety. The carbohydrate moiety can be in the form of a monosaccharide, disaccharide(s). oligosaccharide(s), polysaccharide(s), or their derivatives (e.g. sulfo- or phospho-substituted). The carbohydrate is linear or branched. The carbohydrate moiety is N-linked or O-linked to a polypeptide. N-linked glycosylation is to the amide nitrogen of asparagine side chains and O-linked glycosylation is to the hydroxy oxygen of serine and threonine side chains.

The carbohydrate moiety is endogenous to the subject being vaccinated. Alternatively, the carbohydrate moiety is exogenous to the subject being vaccinated. The carbohydrate moiety is a carbohydrate moiety that is not expressed on polypeptides of the subject being vaccinated. For example, the carbohydrate moieties are plant-specific carbohydrates. Plant specific carbohydrate moieties include for example N-linked glycan having a core bound α1,3 fucose or a core bound β1,2 xylose. Alternatively, the carbohydrate moiety are carbohydrate moieties that are expressed on polypeptides or lipids of the subject being vaccinate. For example, many host cells have been genetically engineered to produce human proteins with human-like sugar attachments.

The subject is at risk of developing or suffering from a viral infection. For example, the subject has traveled to regions or countries in which other SARS-CoV2 infections have been reported.

The methods described herein lead to a reduction in the severity or the alleviation of one or more symptoms of a viral infection. Infections are diagnosed and or monitored by a physician using standard methodologies. A subject requiring immunization is identified by methods know in the art. For example, subjects are immunized as outlined in the CDC's General Recommendation on Immunization (51(RR02) ppl-36).

The subject is e.g., any mammal, e.g., a human, a primate, mouse, rat, dog, cat, camel, cow, horse, pig, a fish or a bird.

The treatment is administered prior to diagnosis of the infection. Alternatively, treatment is administered after diagnosis. Efficaciousness of treatment is determined in association with any known method for diagnosing or treating the disorder or infection. Alleviation of one or more symptoms of the disorder indicates that the compound confers a clinical benefit.

### Methods of Treatment

As used herein, the terms “treat” or “treatment” refer to both therapeutic treatment and prophylactic or preventative measures, wherein the object is to prevent or slow down (lessen) an undesired physiological change or disorder, such as the progression of COVID. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment include those already with the condition or disorder as well as those prone to have the condition or disorder or those in which the condition or disorder is to be prevented. The invention provides for both prophylactic and therapeutic methods of treating a subject at risk of (or susceptible to) a SARS-CoV2-related disease or disorder.

### Prophylactic Methods

In one aspect, the invention provides methods for preventing a SARS-CoV2-related disease or disorder in a subject by administering to the subject a monoclonal antibody of the invention or an agent identified according to the methods of the invention. For example, monoclonal antibodies of the invention, and any variants thereof, can be administered in therapeutically effective amounts. Optionally, two or more anti-SARS-CoV2 antibodies are co-administered. For example, the invention provides for both prophylactic and therapeutic methods of treating a subject at risk of (or susceptible to) COVID.

Subjects at risk for a SARS-CoV2-related diseases or disorders include patients who have been exposed to the SARS-CoV2. For example, the subjects have traveled to regions or countries of the world in which other SARS-CoV2 infections have been reported and confirmed. Administration of a prophylactic agent can occur prior to the manifestation of symptoms characteristic of the SARS-CoV2-related disease or disorder, such that a disease or disorder is prevented or, alternatively, delayed in its progression.

The appropriate agent can be determined based on screening assays described herein. Alternatively, or in addition, the agent to be administered is a monoclonal antibody that neutralizes a SARS-CoV2 that has been identified according to the methods of the invention. In some embodiments, the antibody of the invention can be administered with other antibodies or antibody fragments known to neutralize SARS-CoV2. Administration of said antibodies can be sequential, concurrent, or alternating.

### Therapeutic Methods

Another aspect of the invention pertains to methods of treating a SARS-CoV2-related disease or disorder in a patient. In one embodiment, the method involves administering an agent (e.g., an agent identified by a screening assay described herein and/or monoclonal antibody identified according to the methods of the invention), or combination of agents that neutralize the SARS-CoV2 to a patient suffering from the disease or disorder.

### Combinatory Methods

The invention provides treating a SARS-CoV2-related disease or disorder, in a patient by administering two or more antibodies wherein the Fc region of said variant does not bind or has reduced binding to the Fc gamma receptor, with other SARS-CoV2 neutralizing antibodies known in the art. In another embodiment, the invention provides methods for treating a SARS-CoV2-related disease or disorder in a patient by administering an antibody of the invention with any anti-viral agent known in the art. Anti-viral agents can be peptides, nucleic acids, small molecules, inhibitors, or RNAi.

The invention will be further described in the following examples, which do not limit the scope of the invention described in the claims.

### EXAMPLES

### Example 1

### Purified Phage Binding Curves (RBD-Fc)

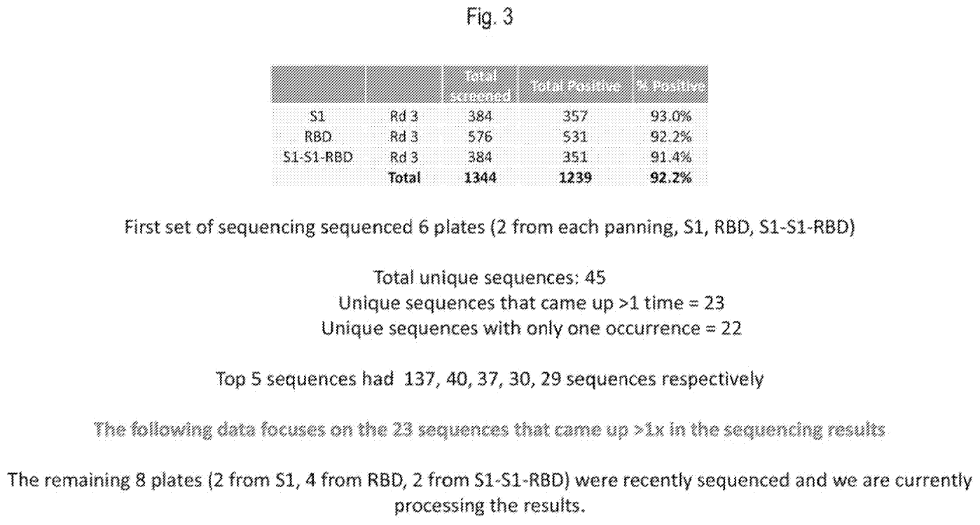

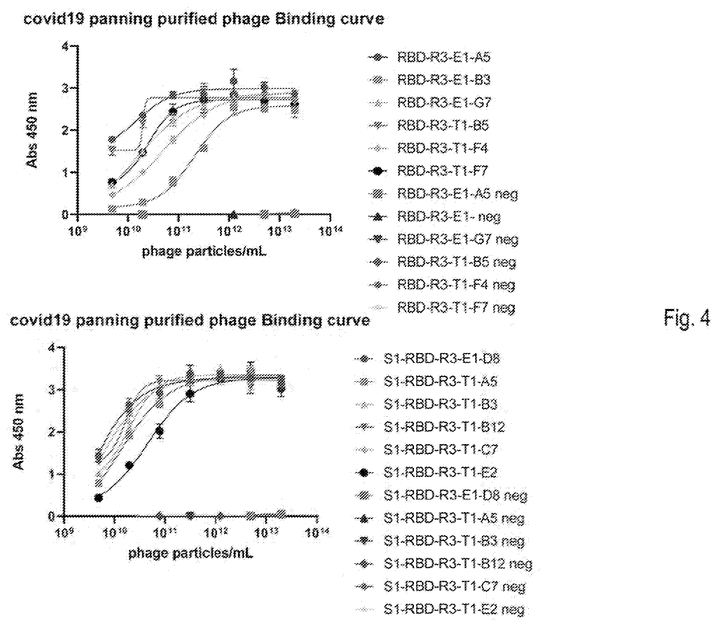

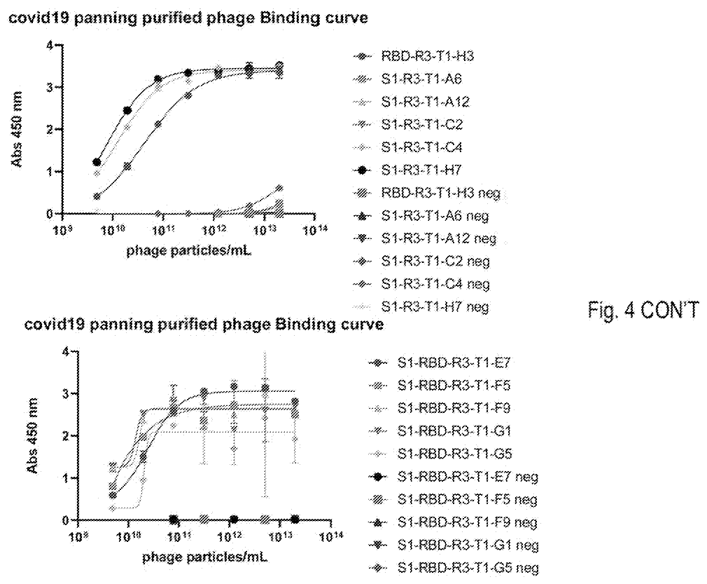

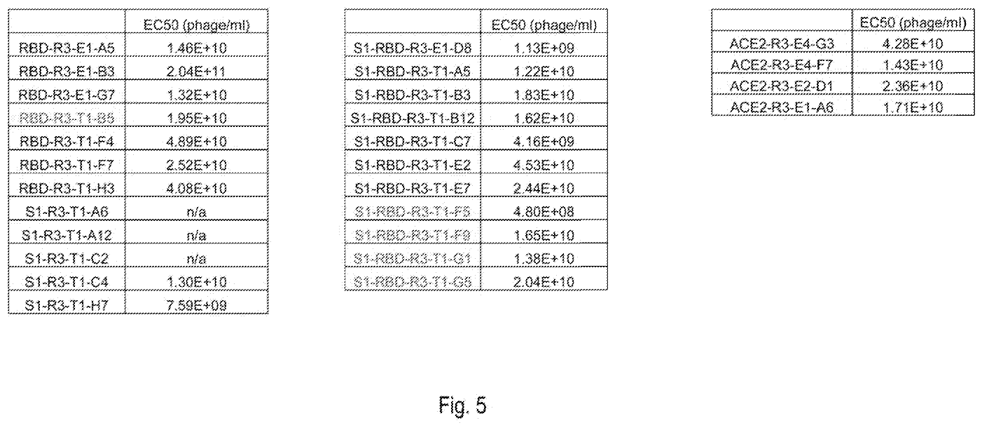

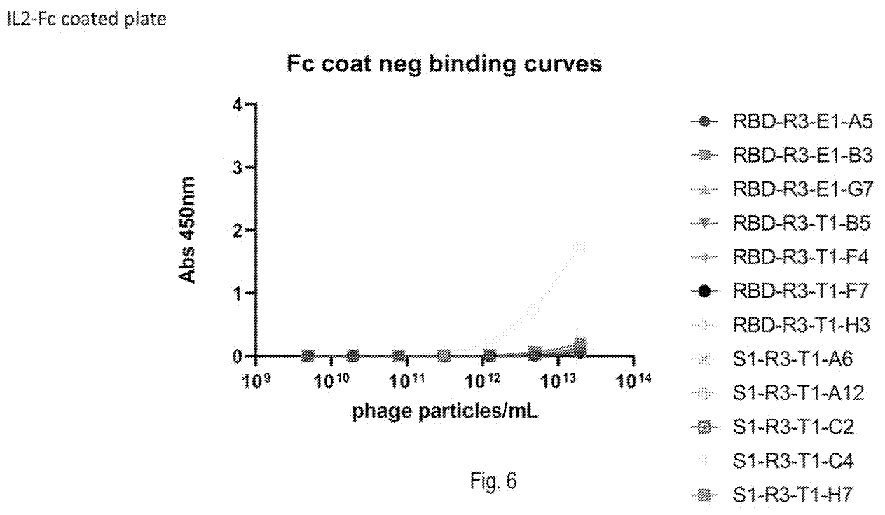

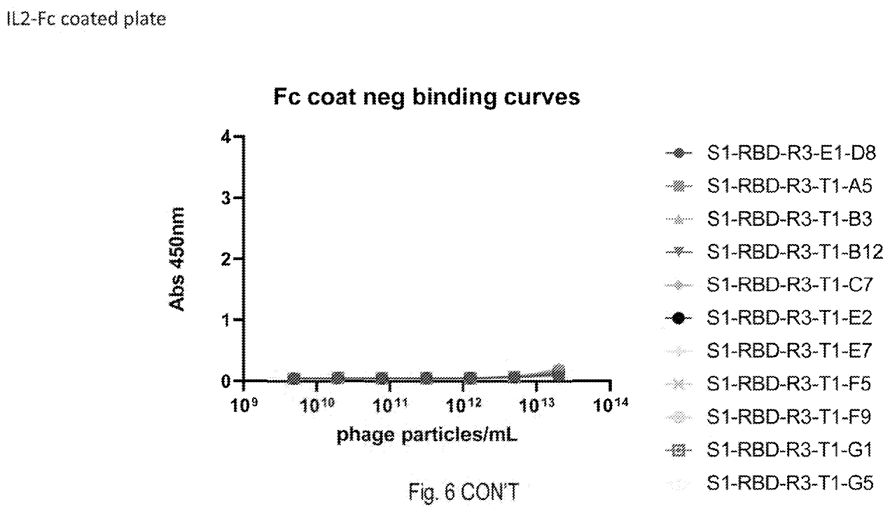

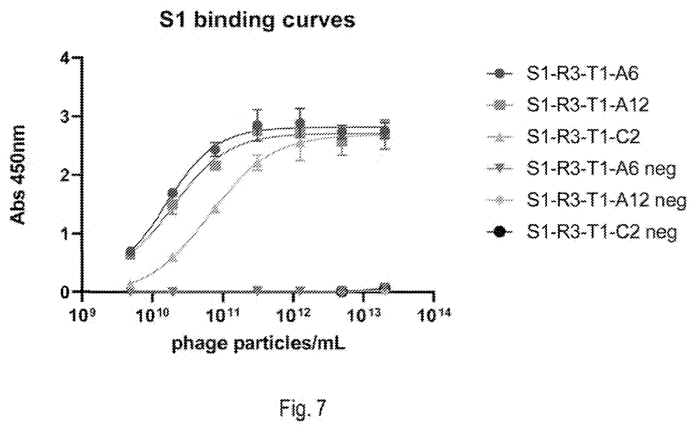

Based on the binding curves of FIG. 3-5, we have antibodies against the RBD with a variety of affinities. There are also 3 clones which do not bind to the RBD. Looking at the sequencing, there were multiple copies of each of these clones, but they only came from S1 panning plates. This indicates that they are S1 specific but not directed to the RBD So binding curves for those 3 were generated against S1 proteins.

### Example 2

Anti-RBD Competition with ACE2

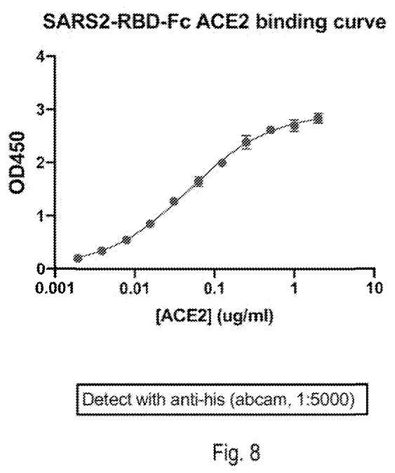

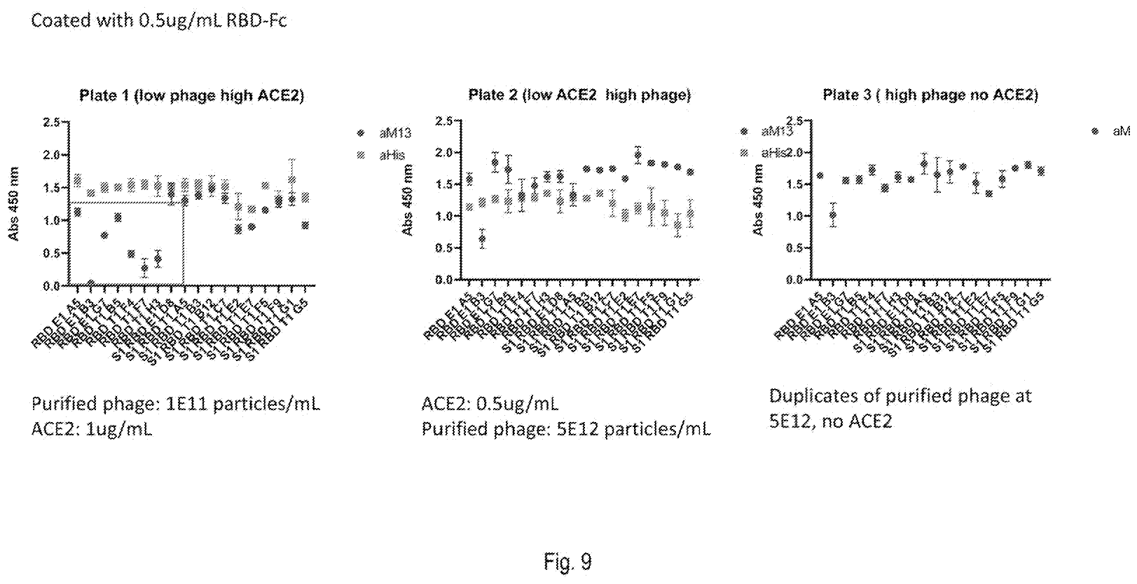

See, for example, FIG. 7-9.

Plates are coated with RBD-Fc at 0.5 ug/ml.

Plate 1: a low concentration of purified phage (on upper shoulder of binding curve) is first added to the plate, before a high concentration of ACE2 (1 μg/ml) is added

Plate 2: a low concentration of ACE2 (0.5 μg/ml) is first added to the plate, before a high concentration of purified phage is added

Samples were run in quadruplicate so that both phage binding (anti-M13) and ACE2 binding (anti-his) can be detected in duplicate

Based on the data shown in FIG. 7-9, for example, three clones were chosen for purified phage competition curves with ACE2.

Plates were coated with 0.5 μg/ml RBD-Fc. A constant amount of phage was added to each well (top shoulder of binding curve) followed by serial dilutions of ACE2. The remaining phage were then detected by anti-M13.

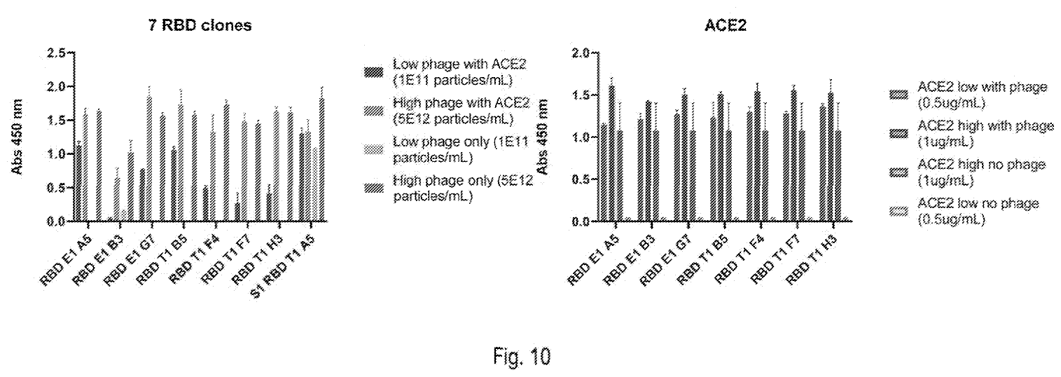

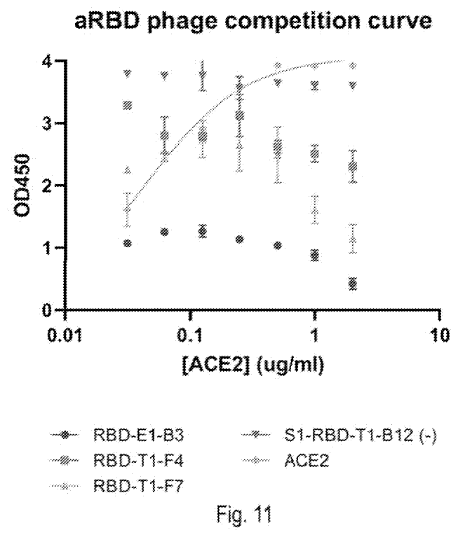

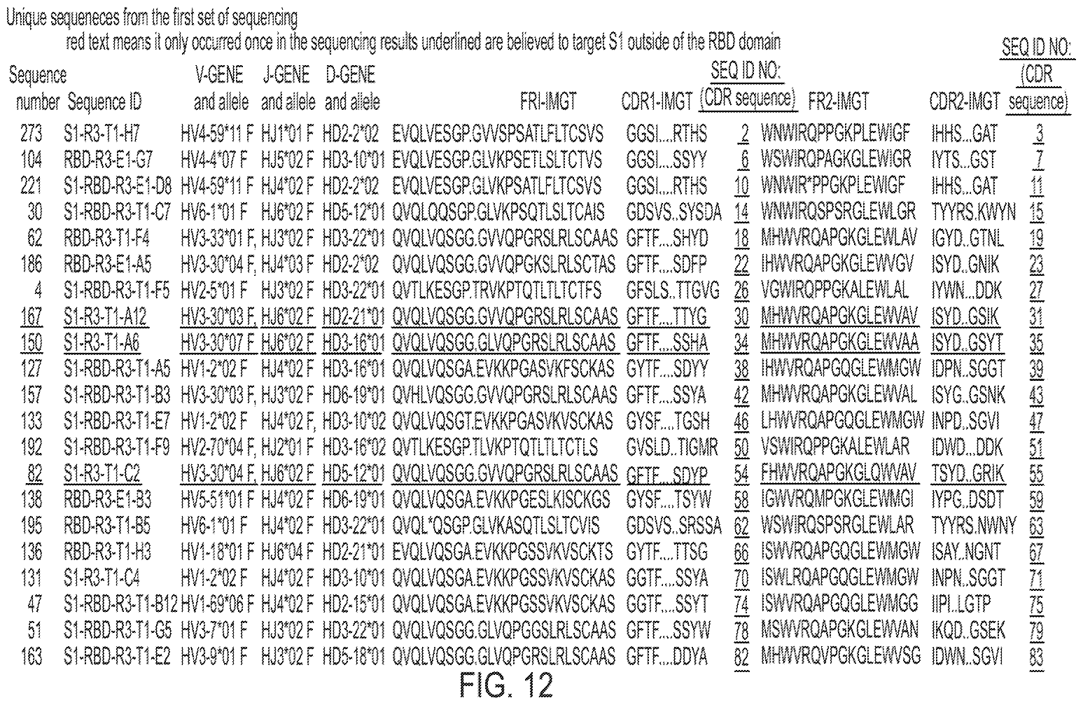

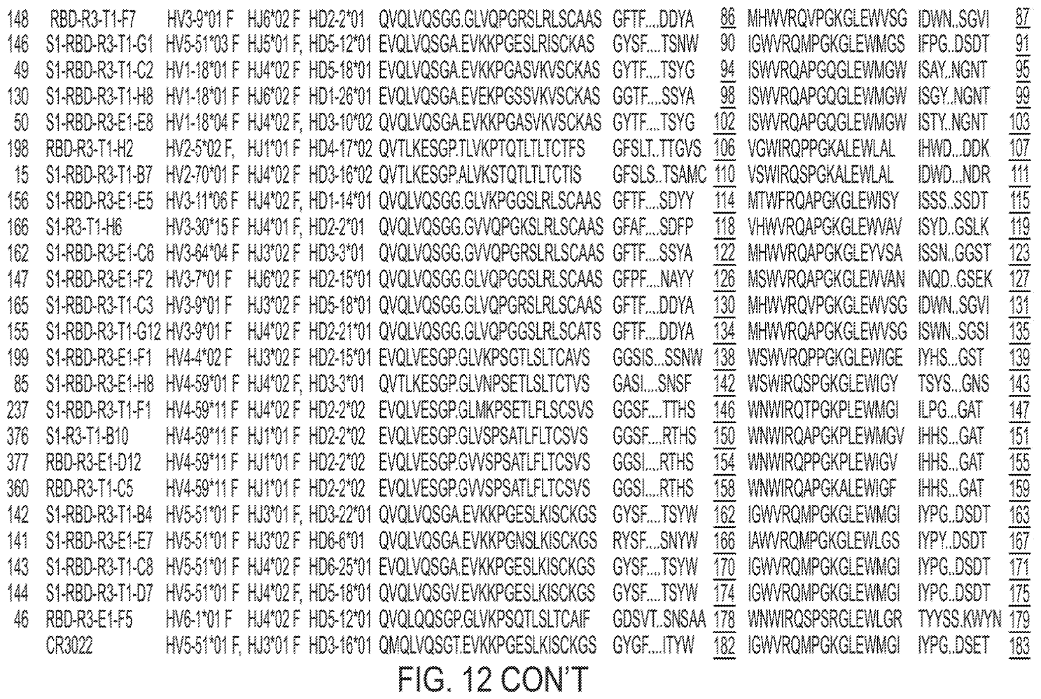

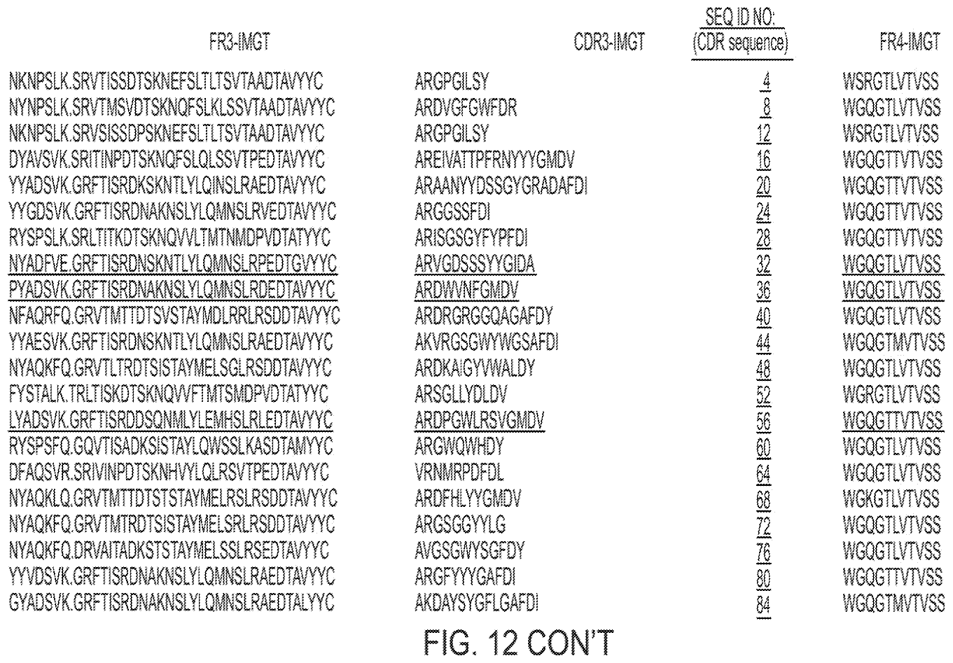

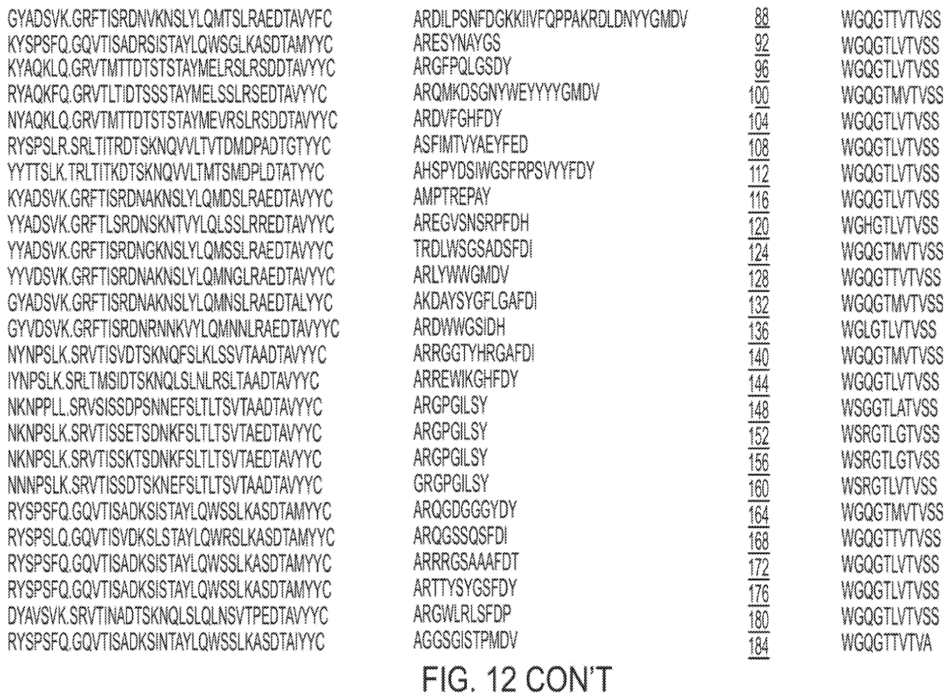

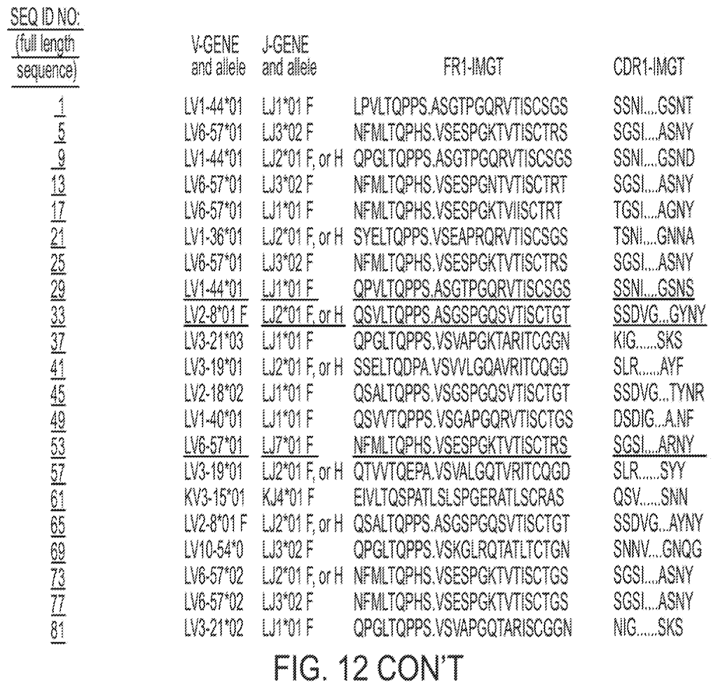

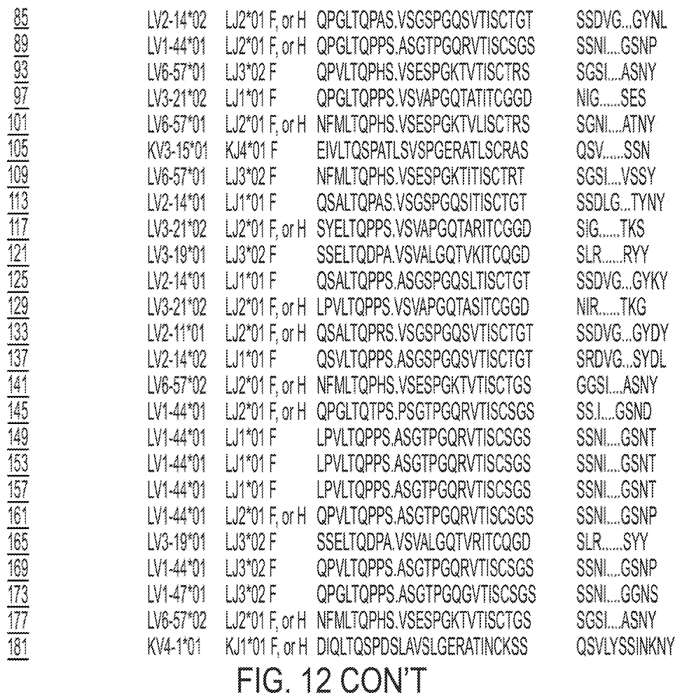

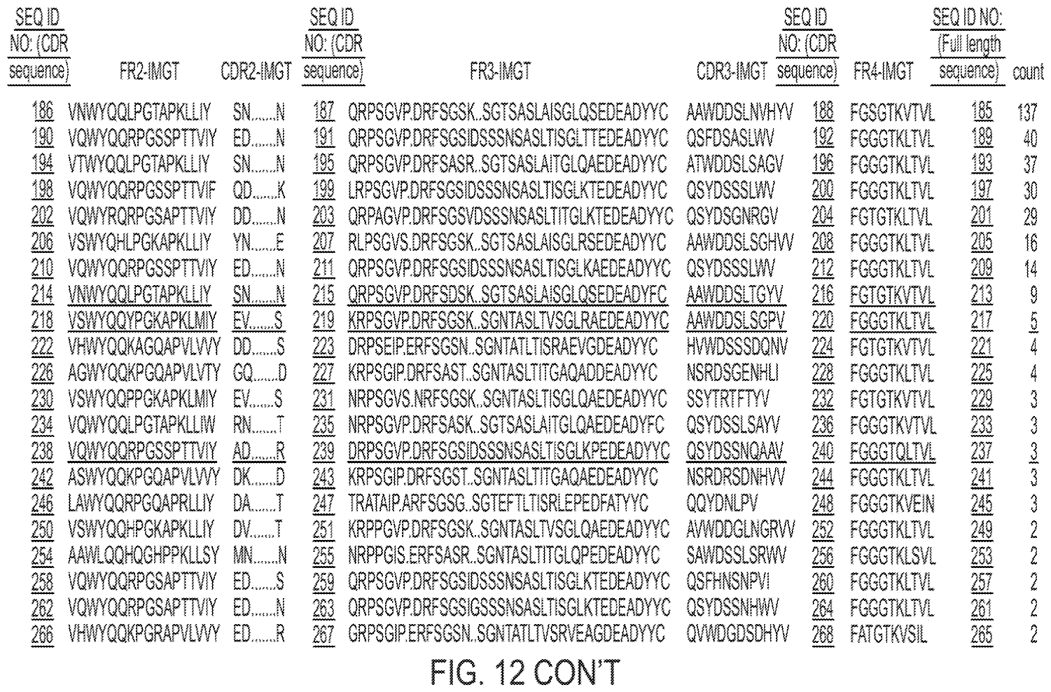

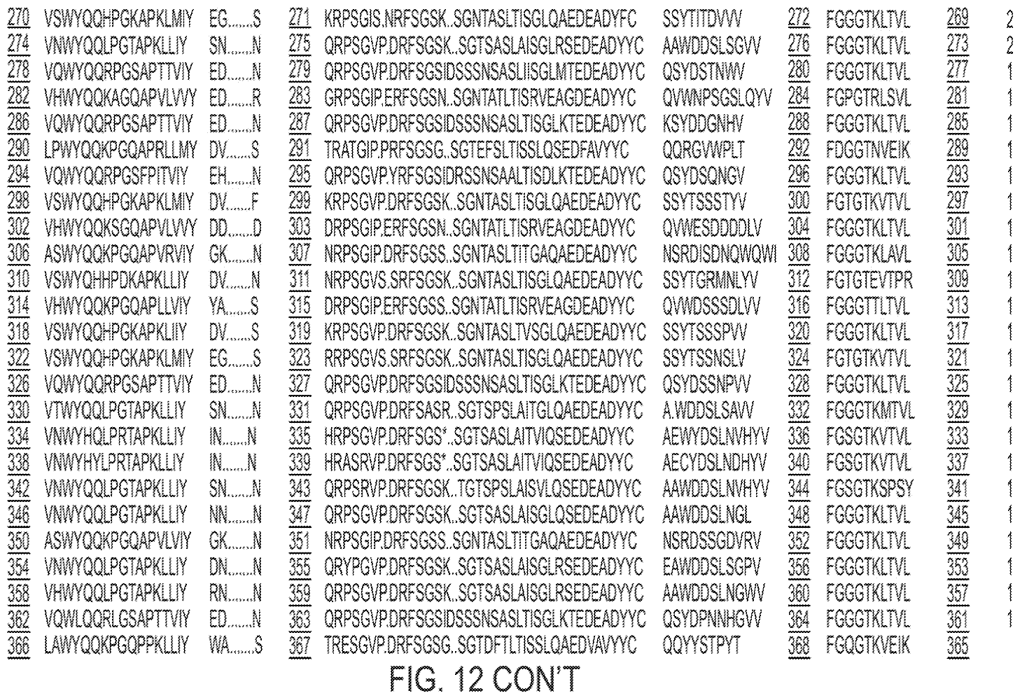

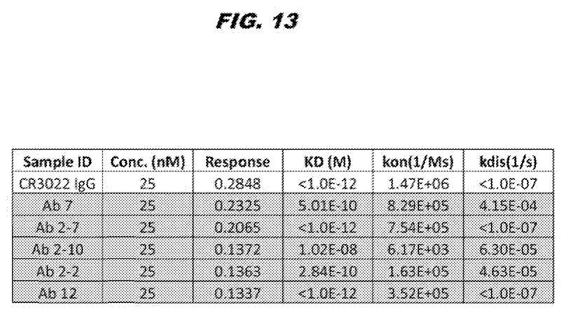

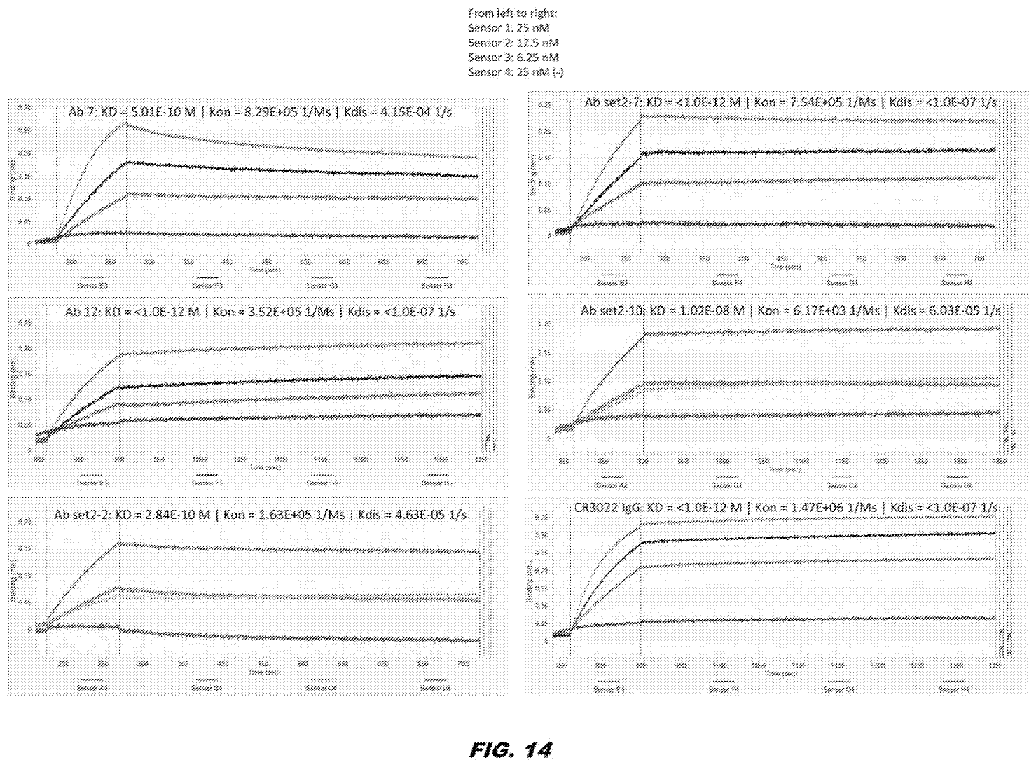

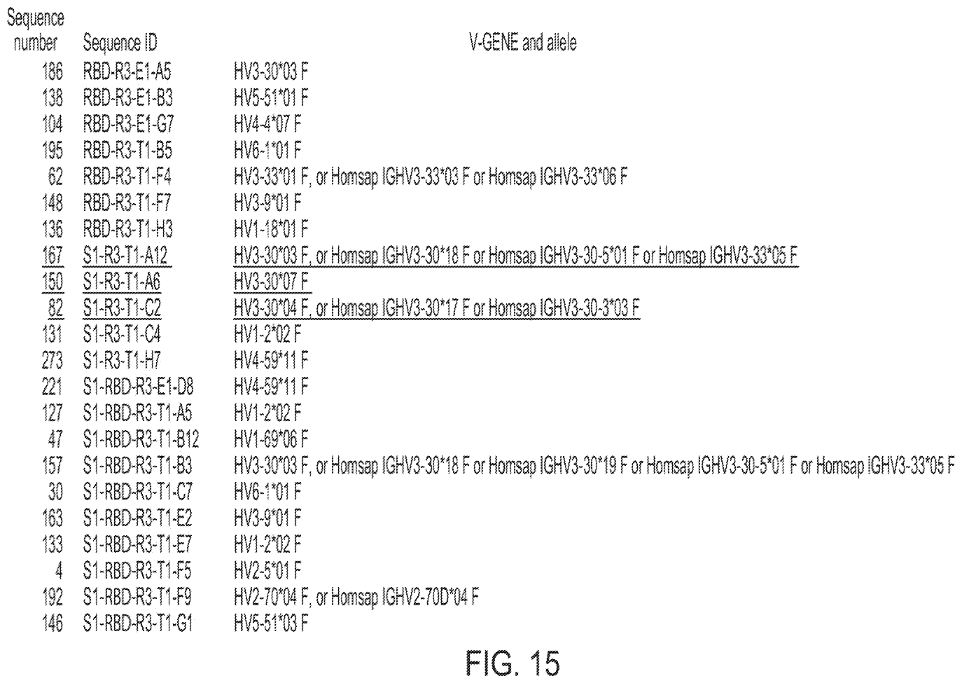

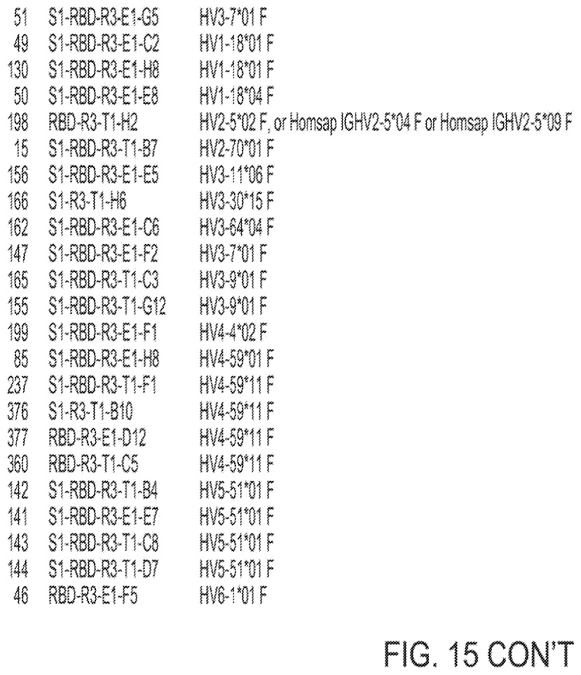

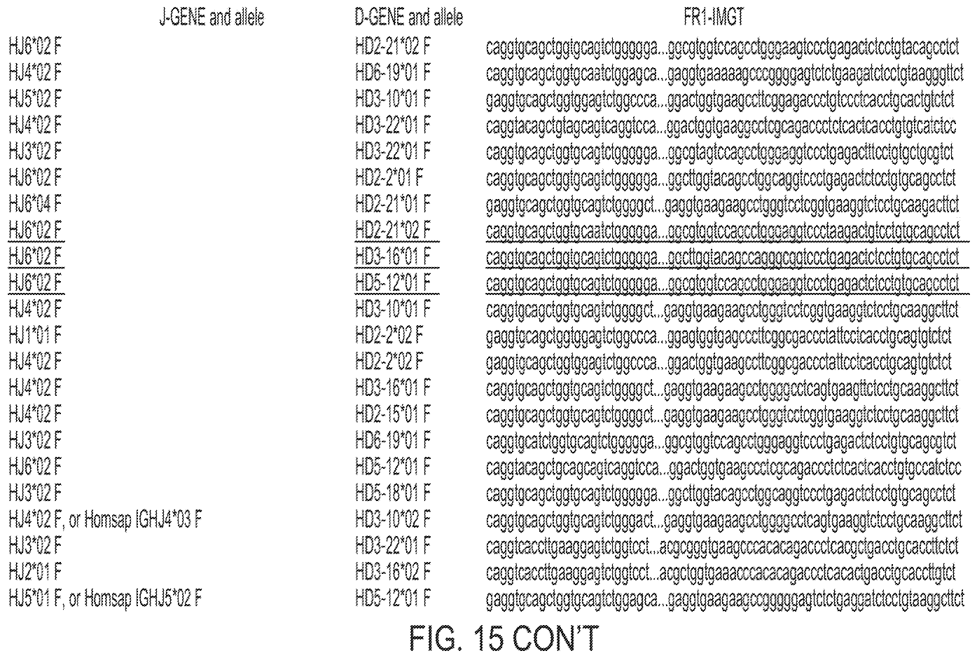

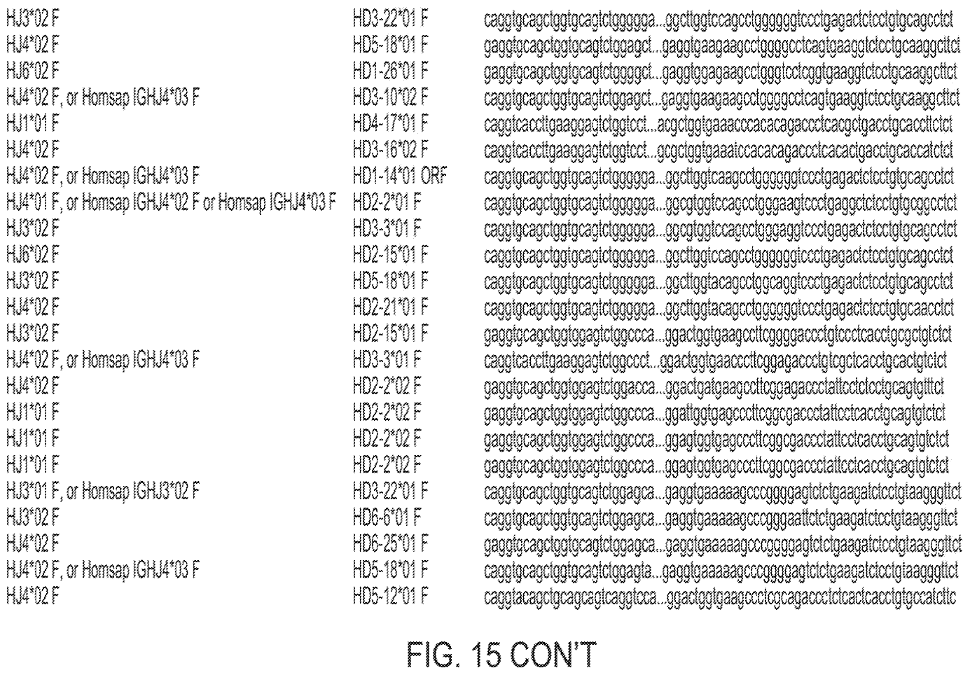

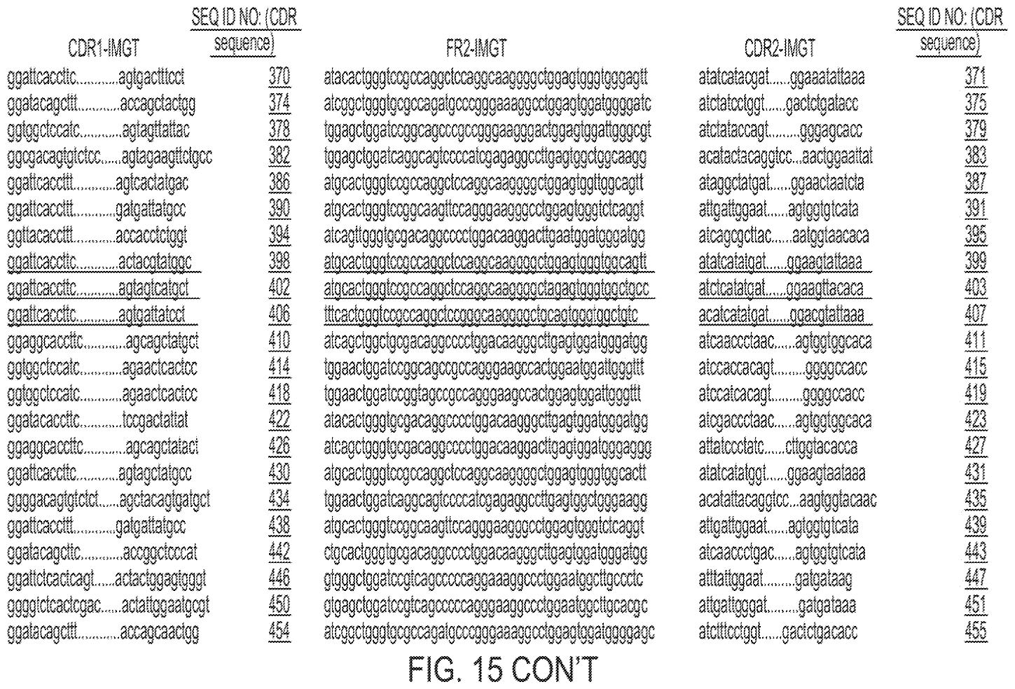

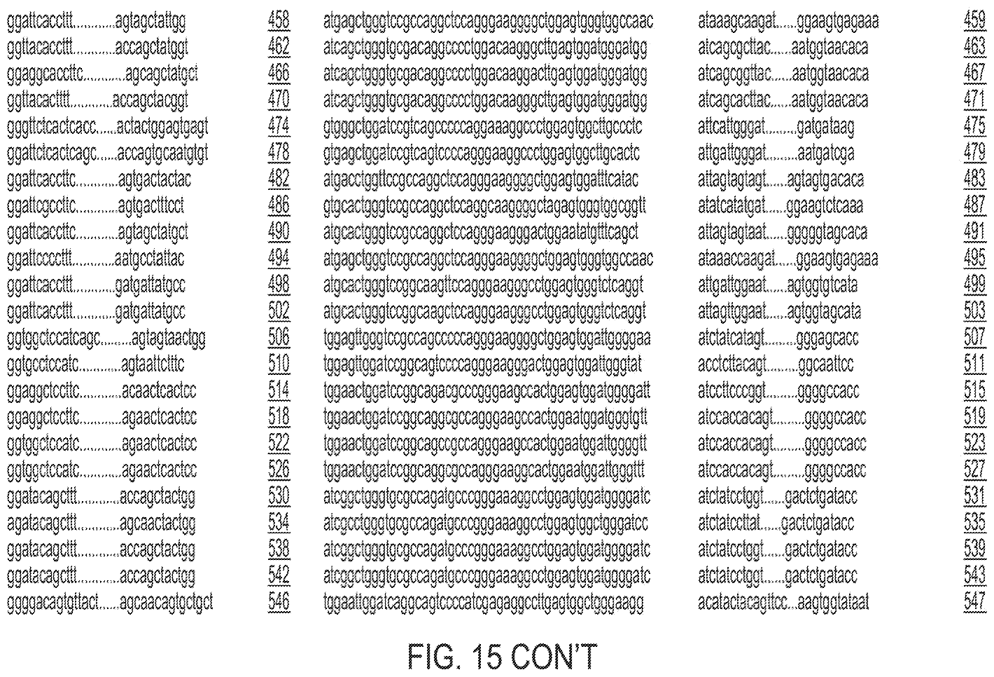

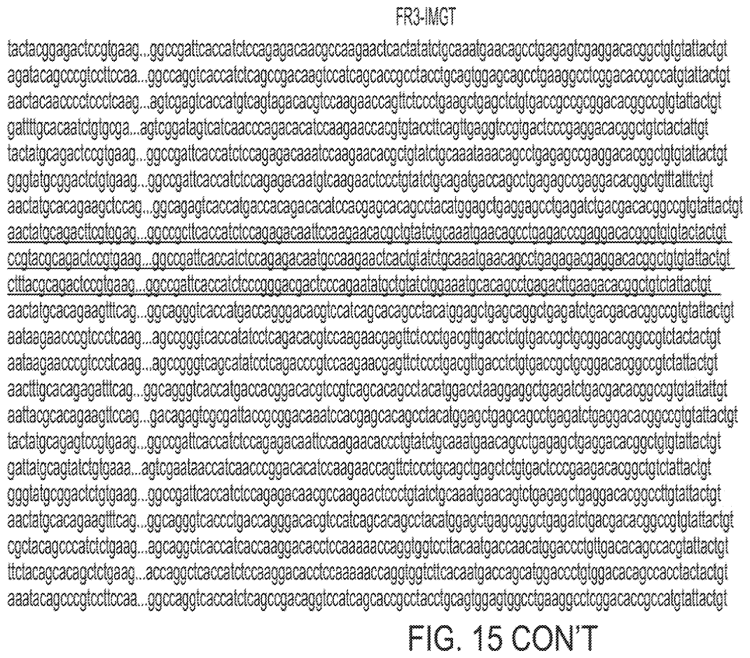

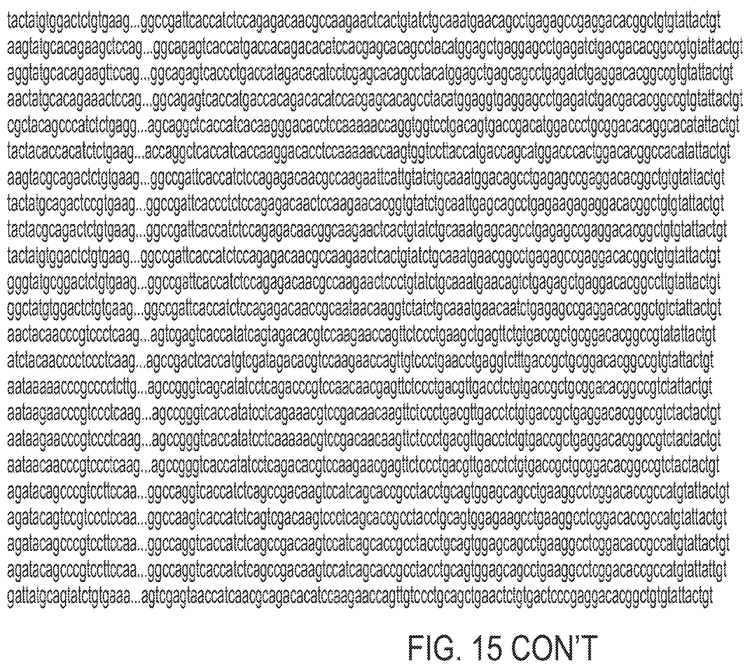

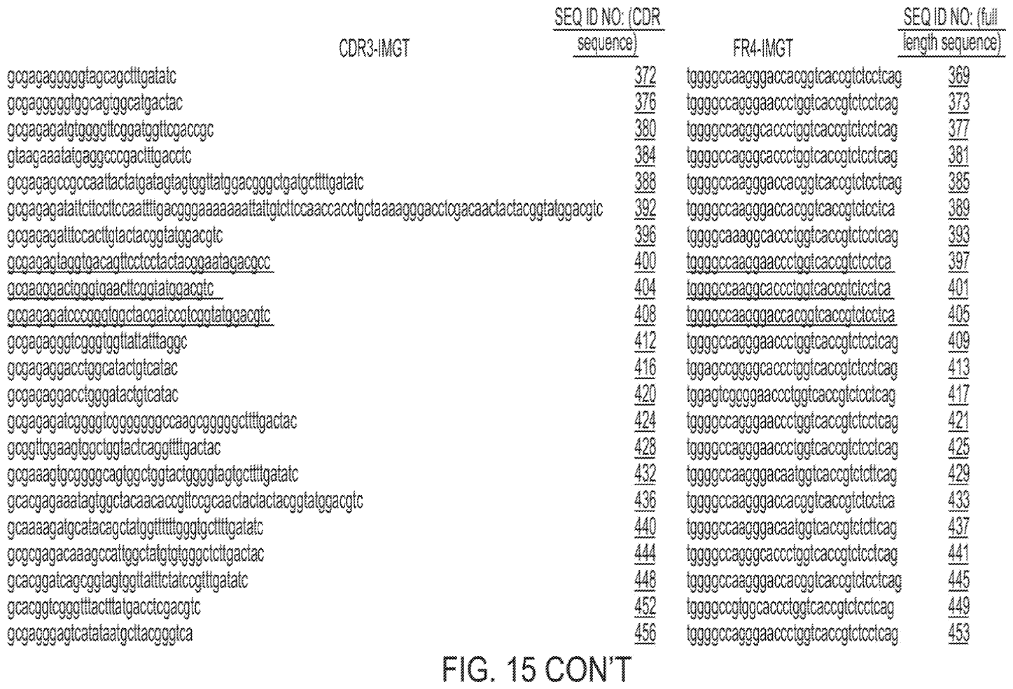

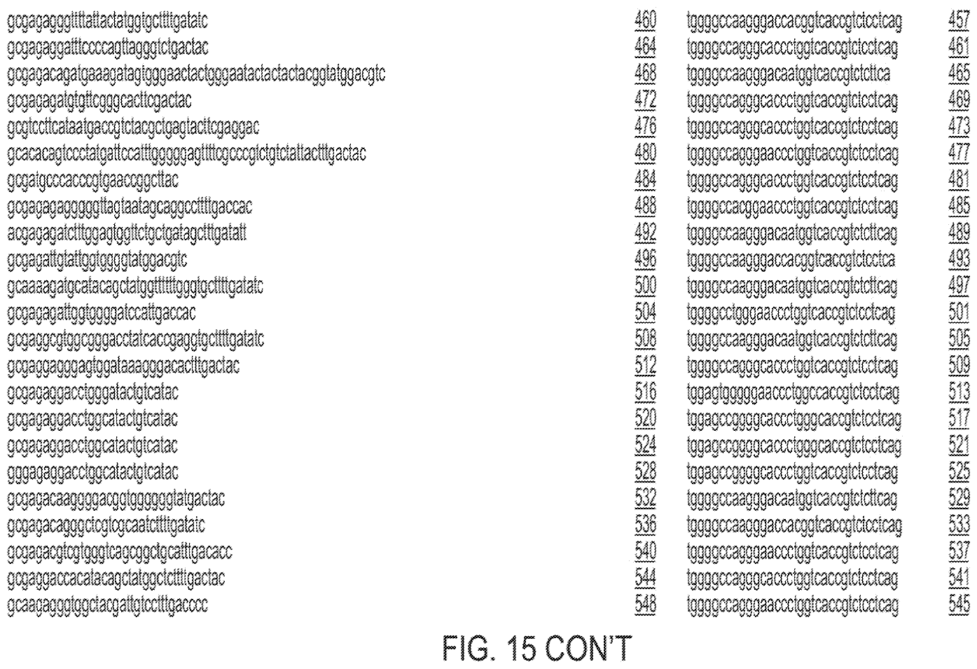

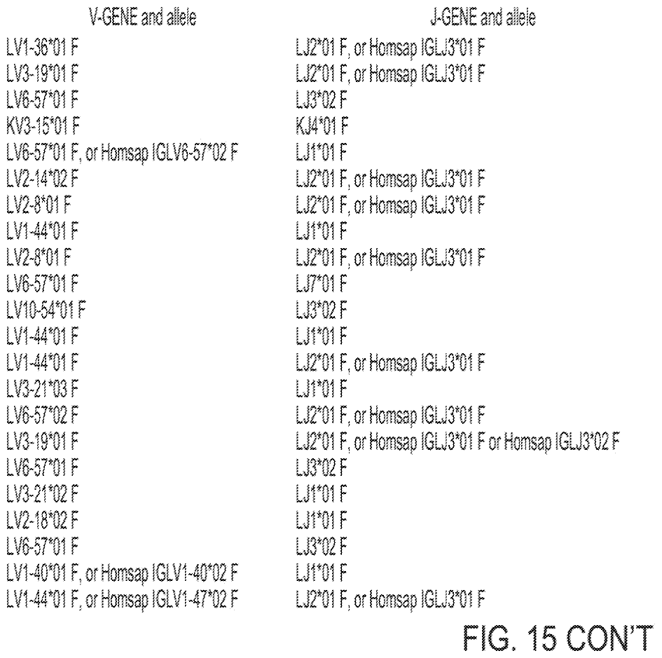

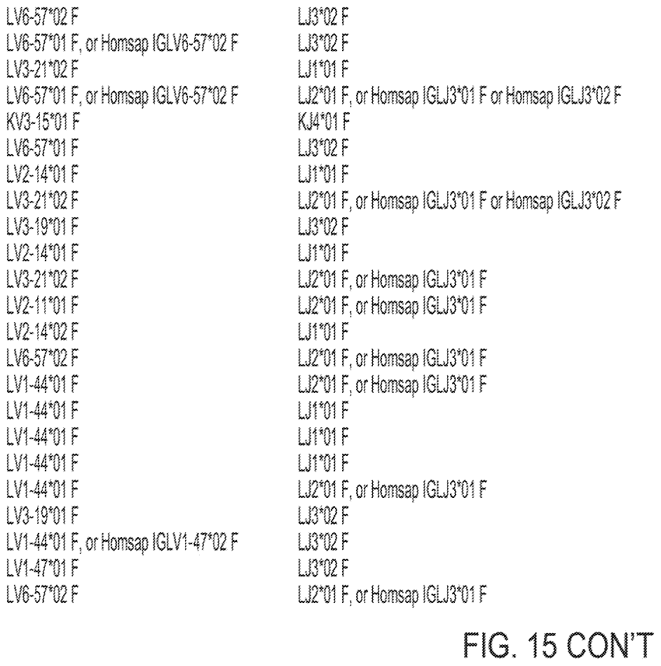

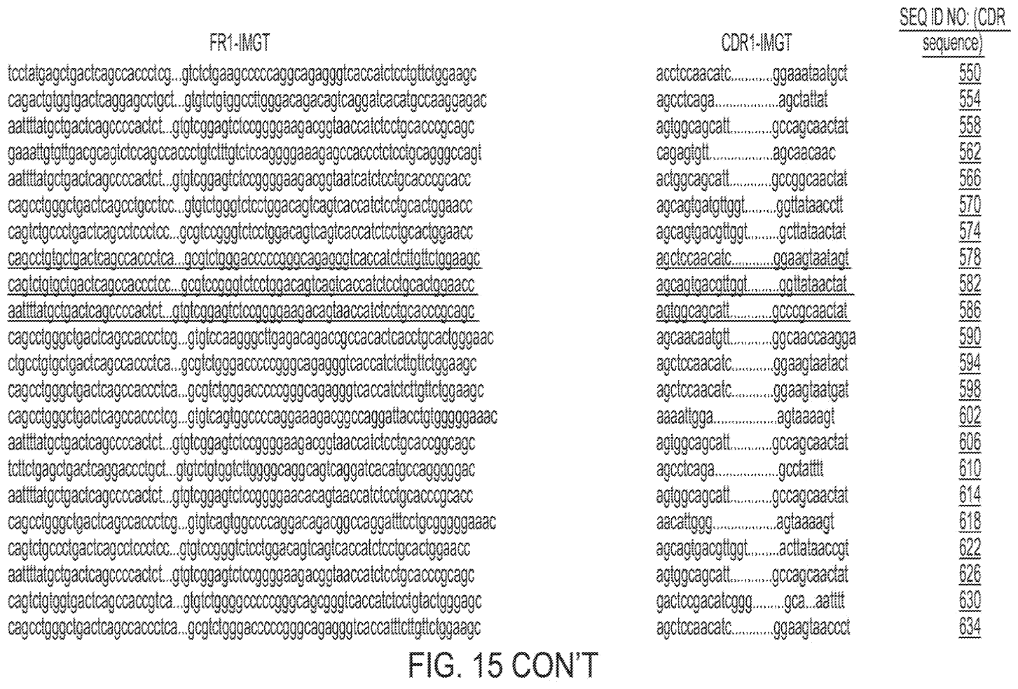

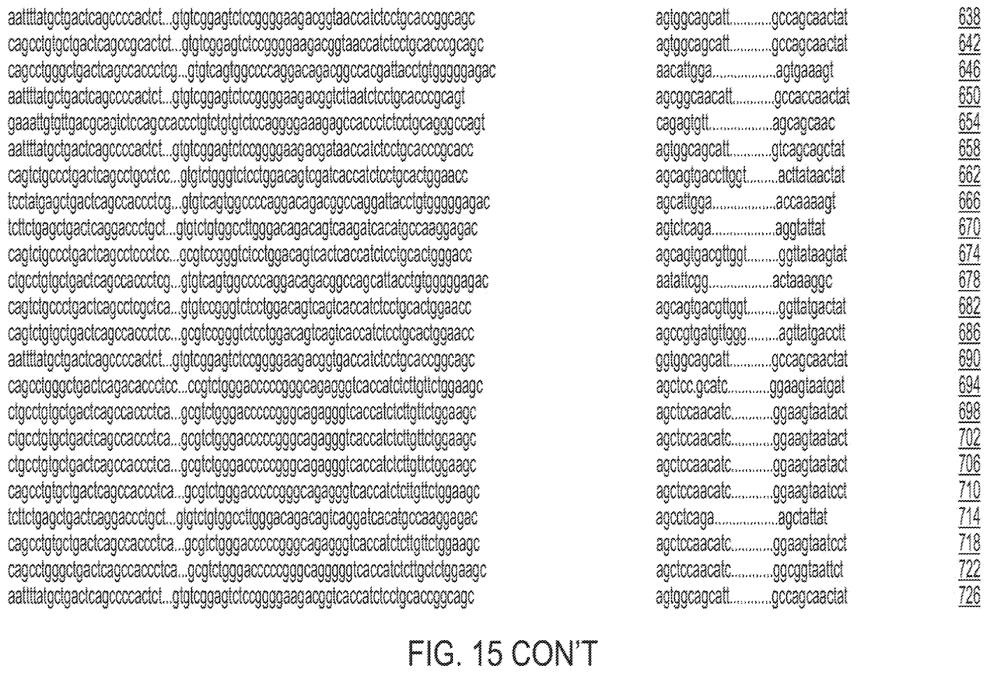

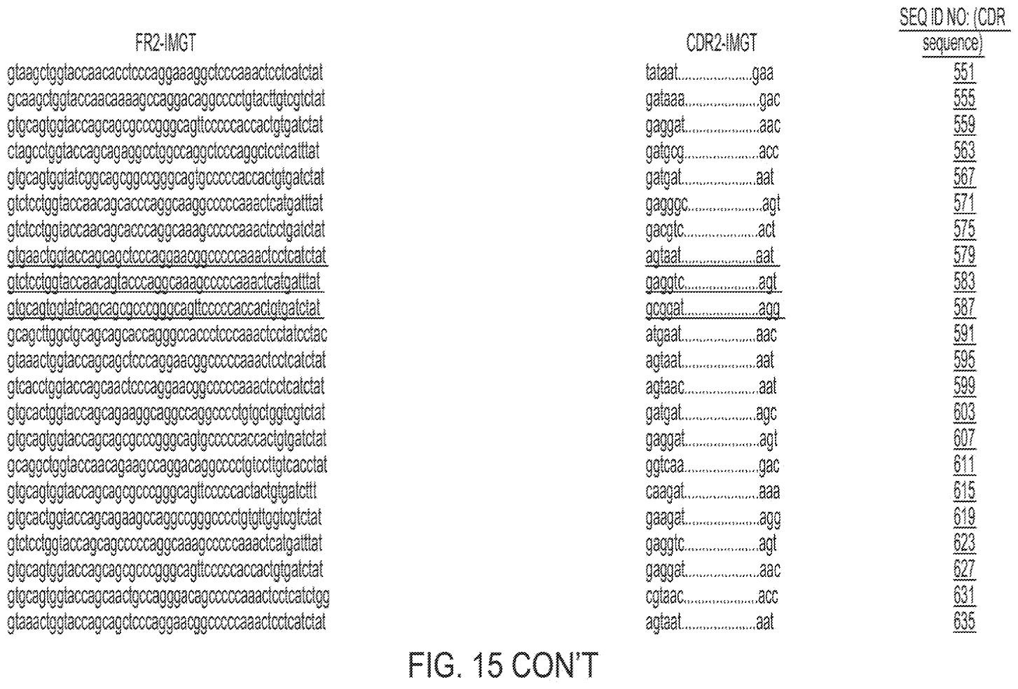

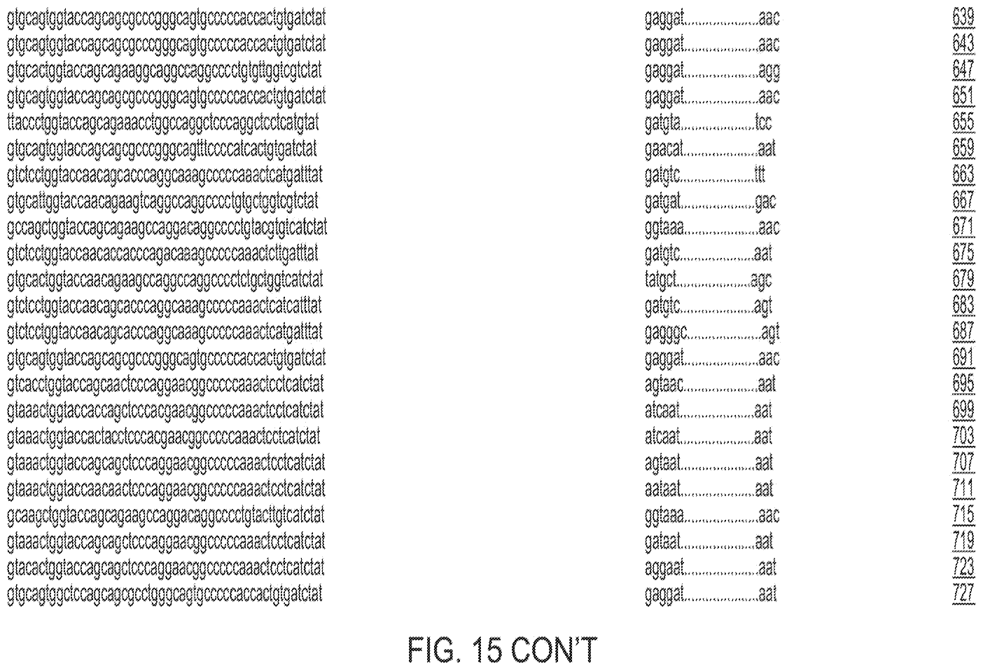

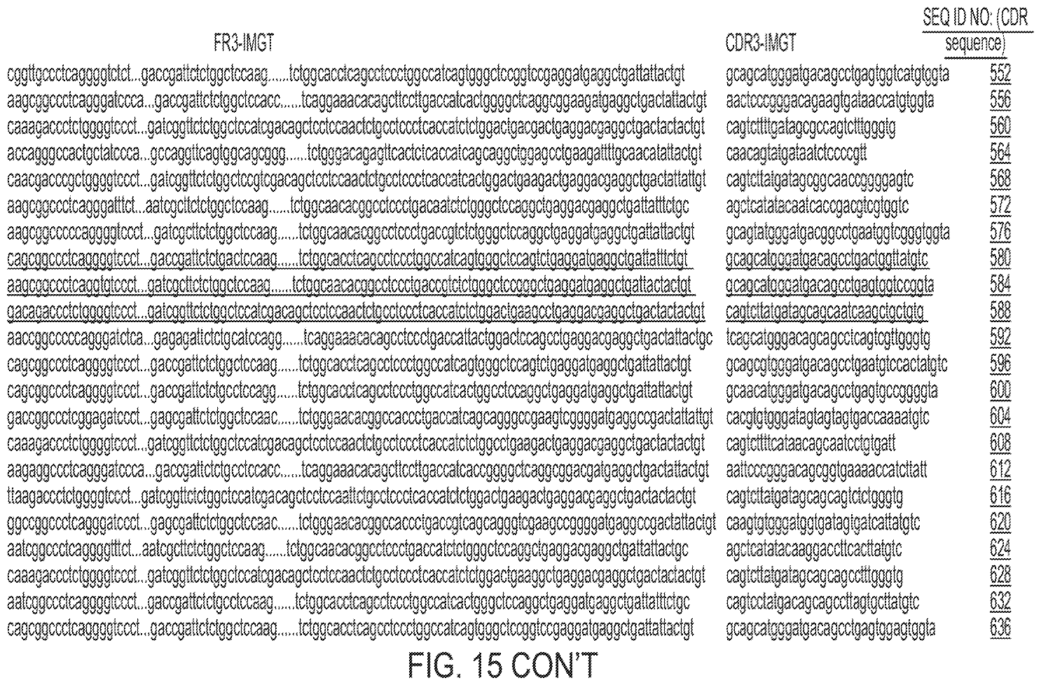

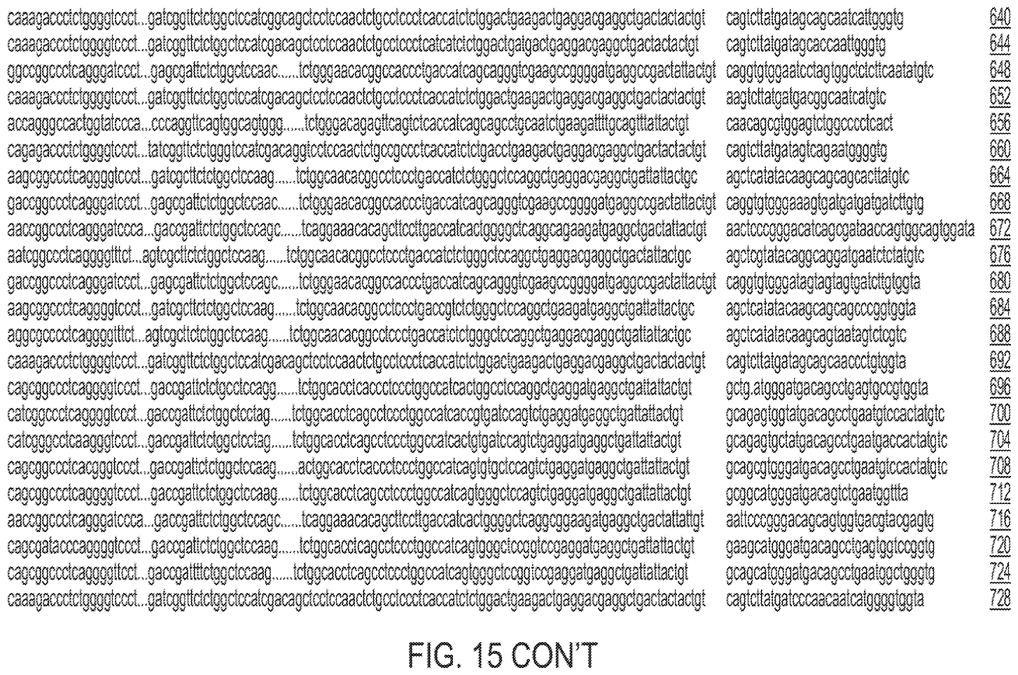

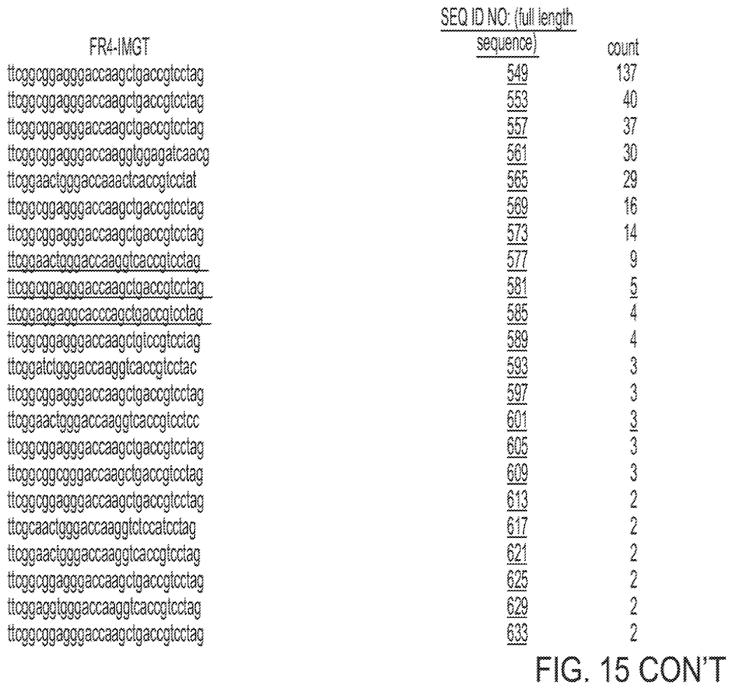

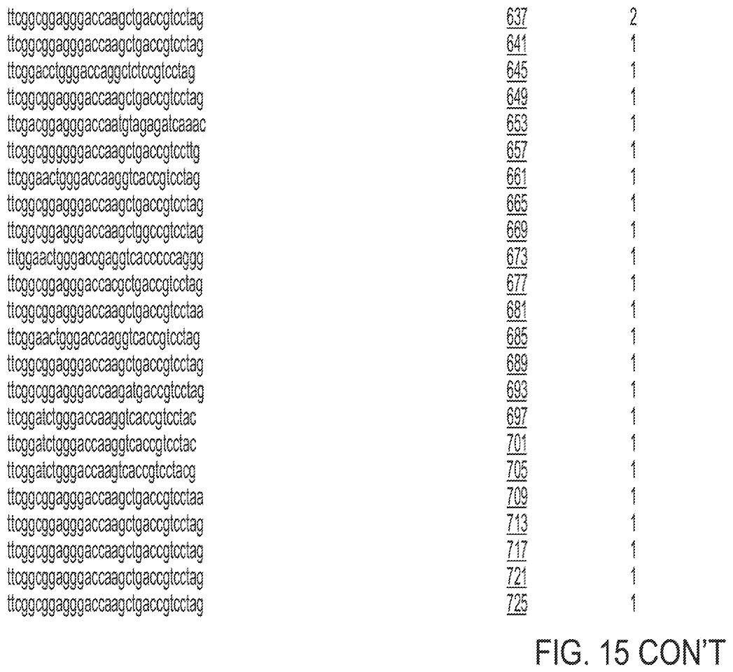

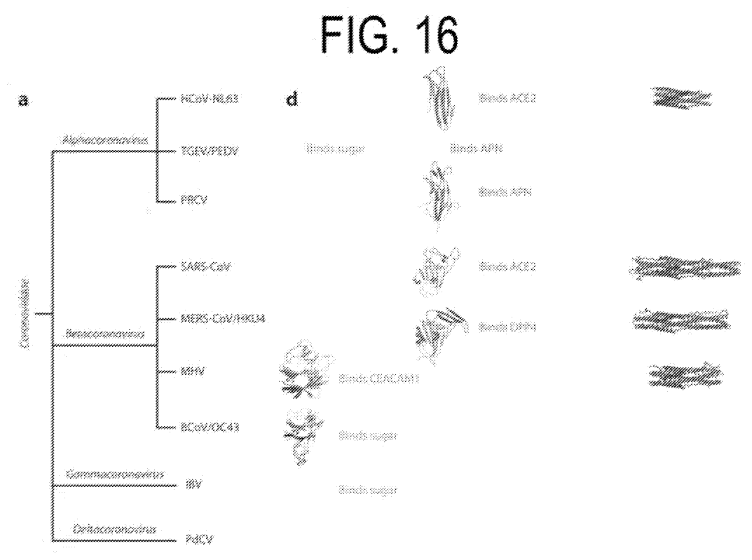

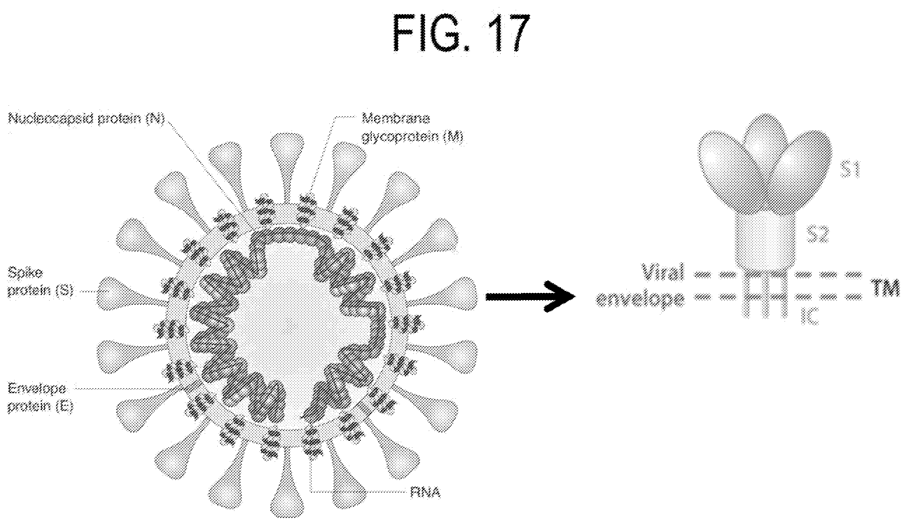

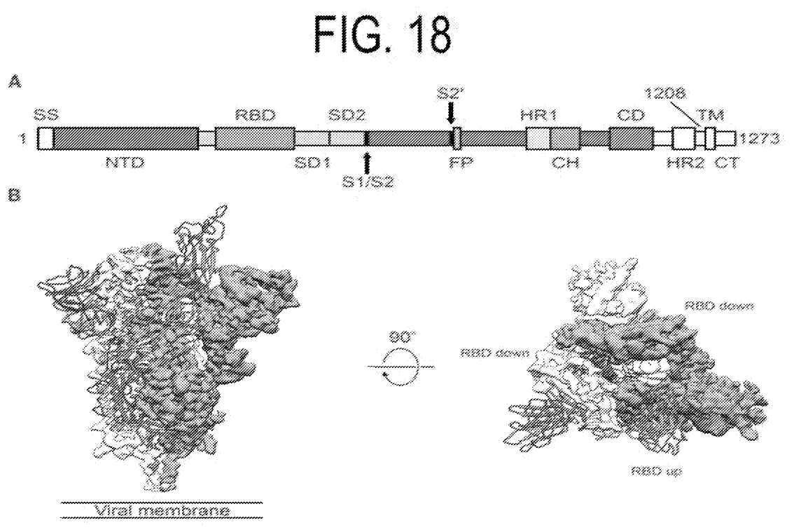

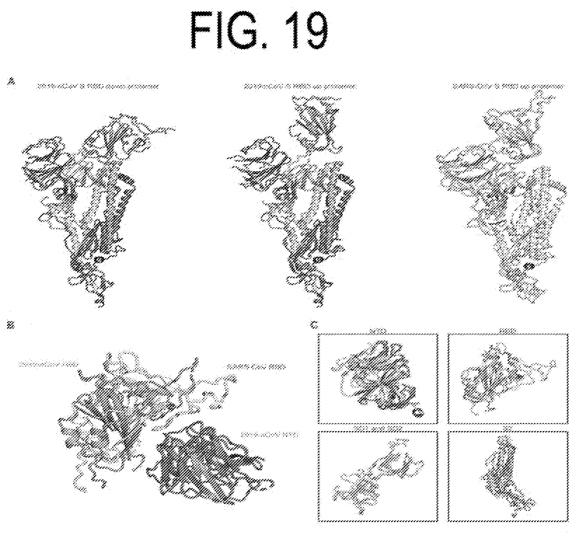

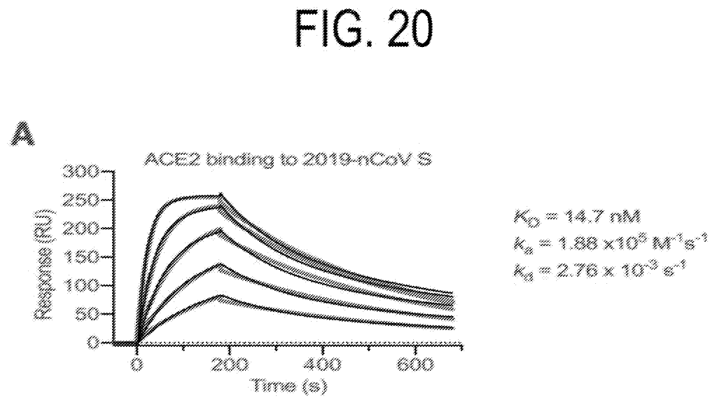

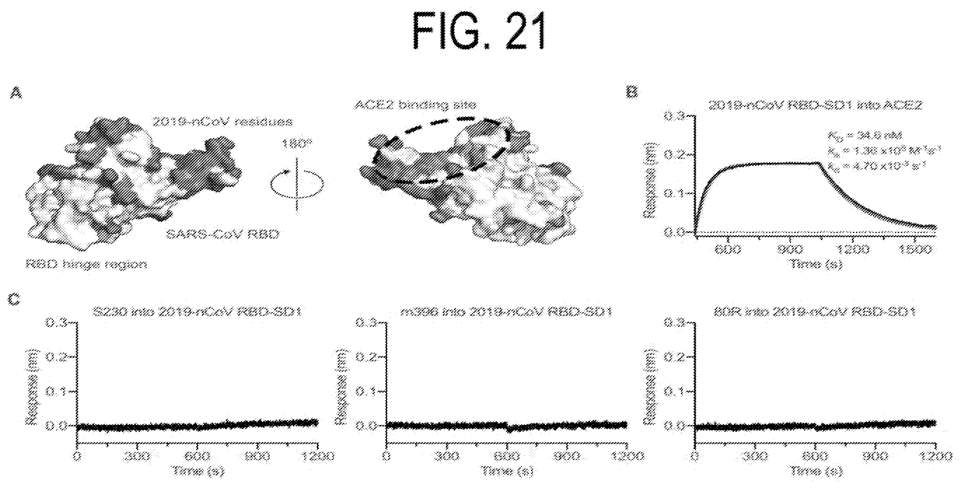

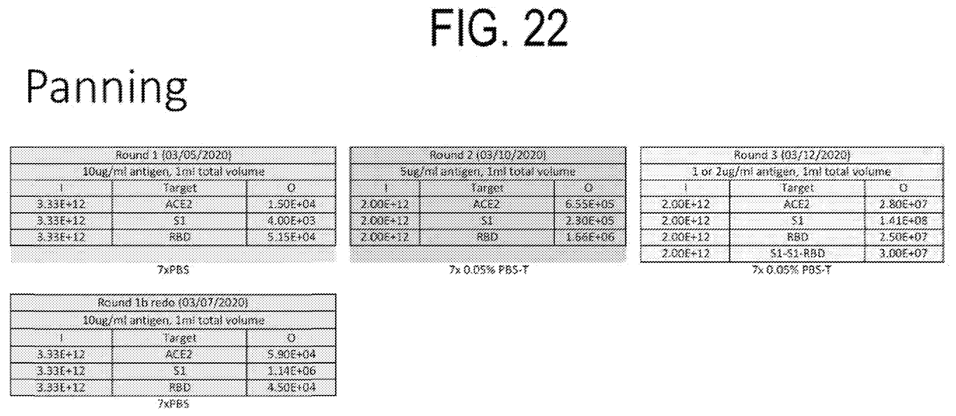

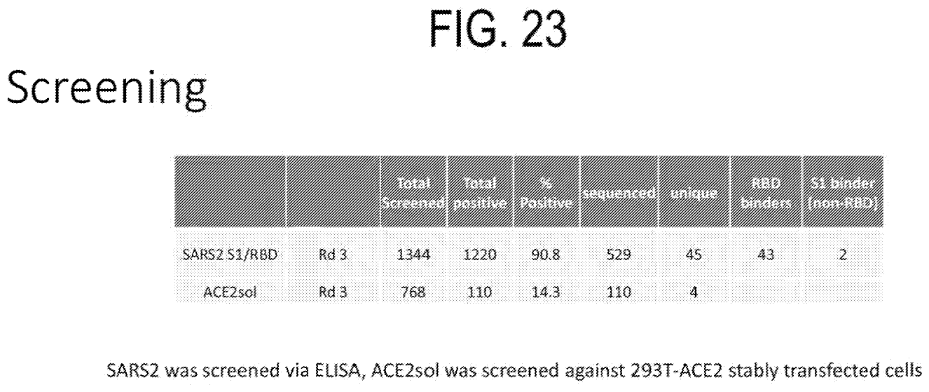

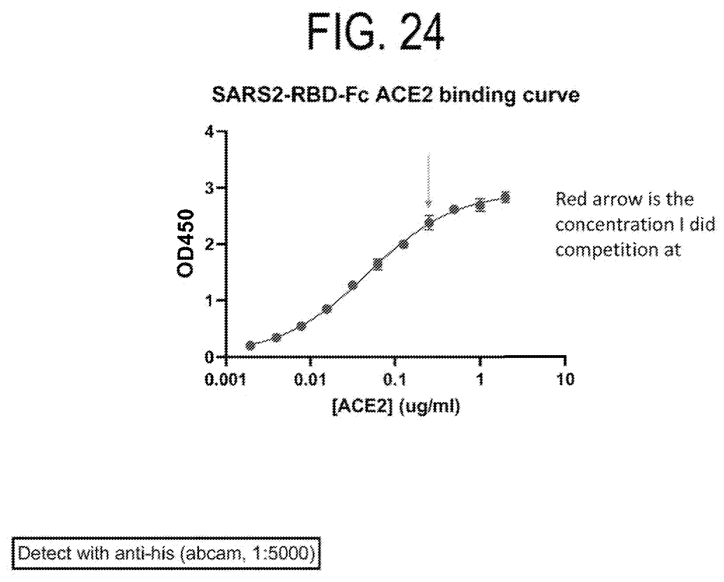

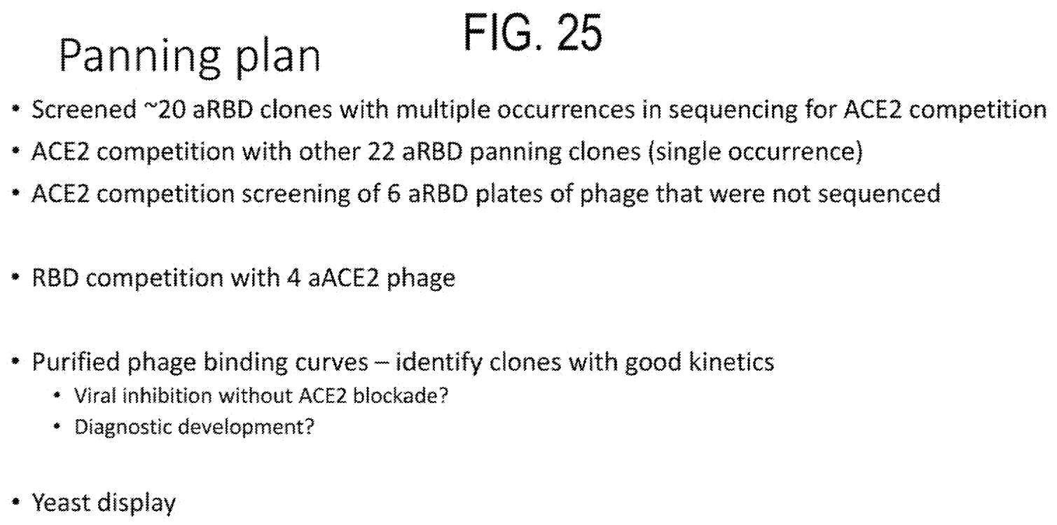

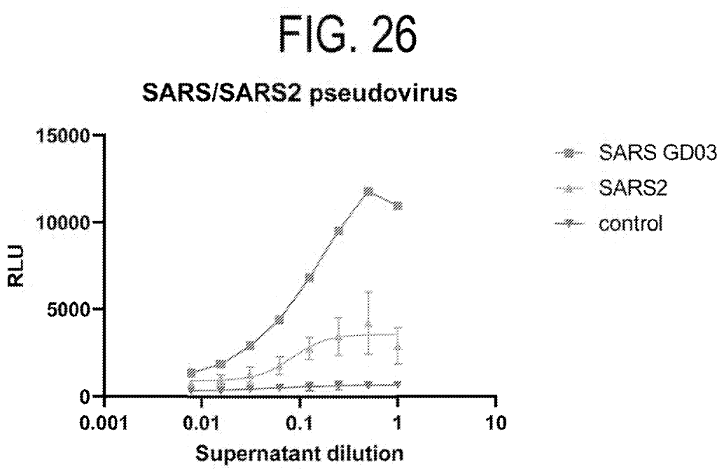

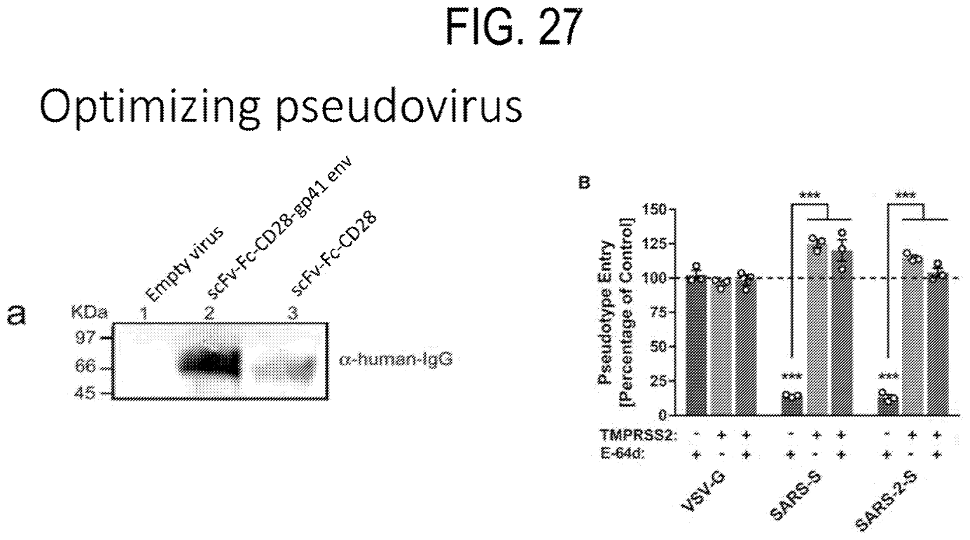

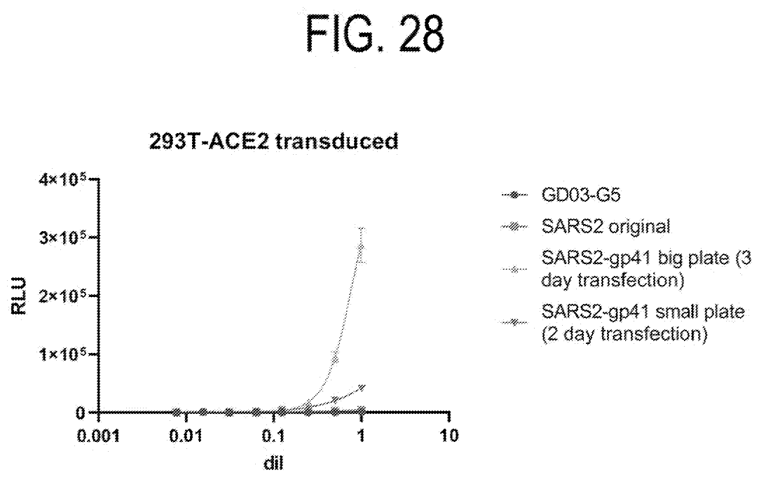

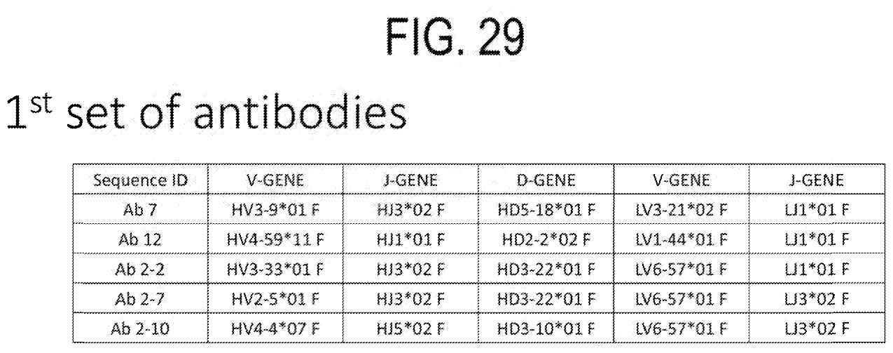

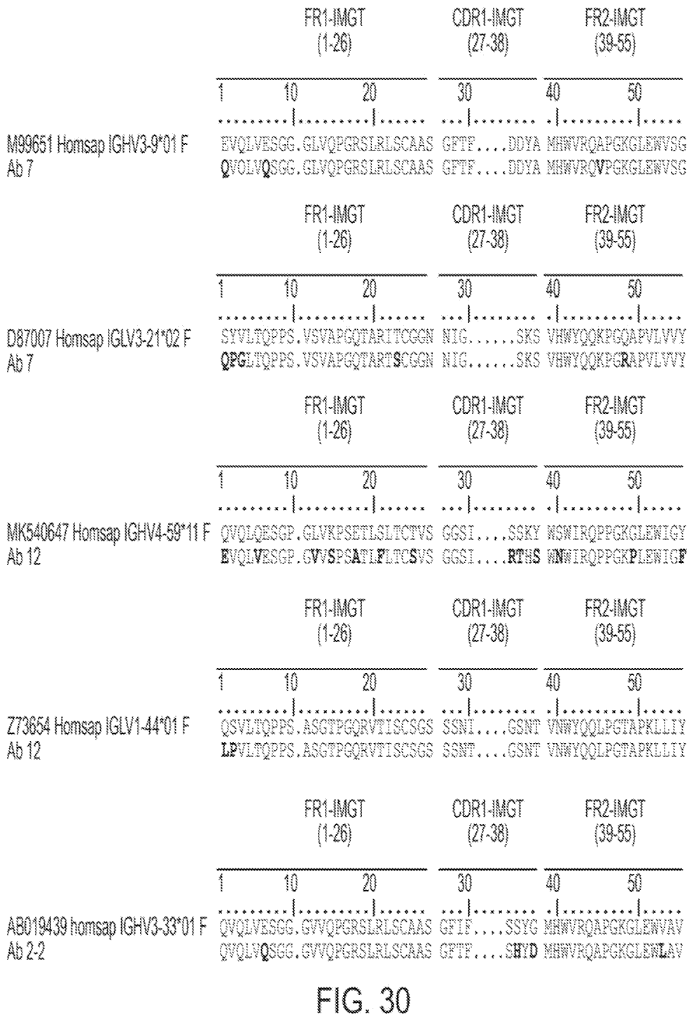

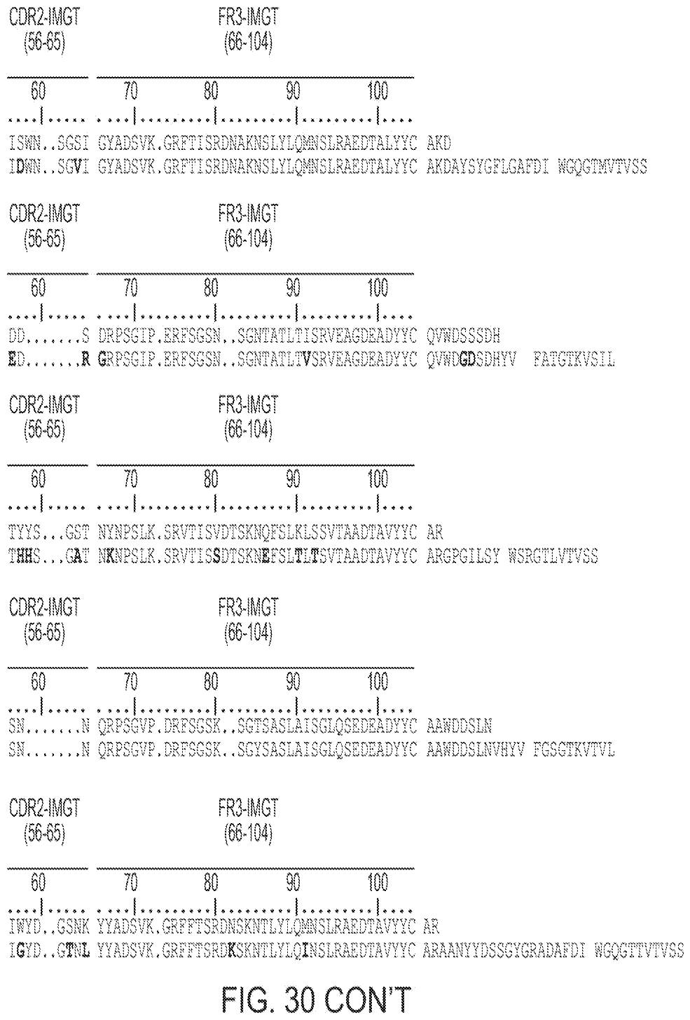

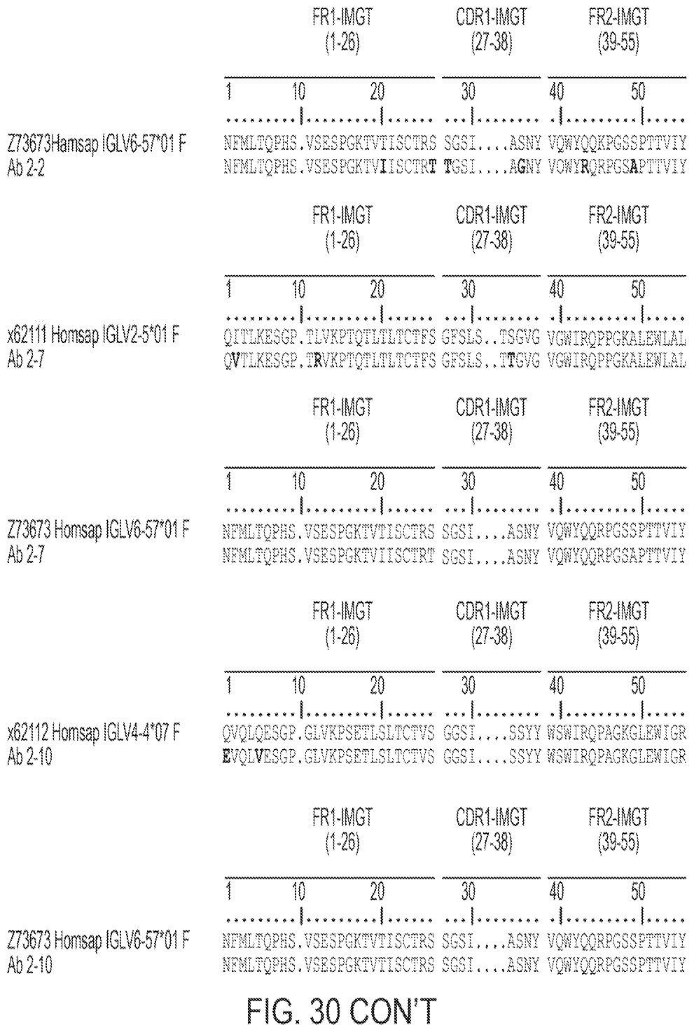

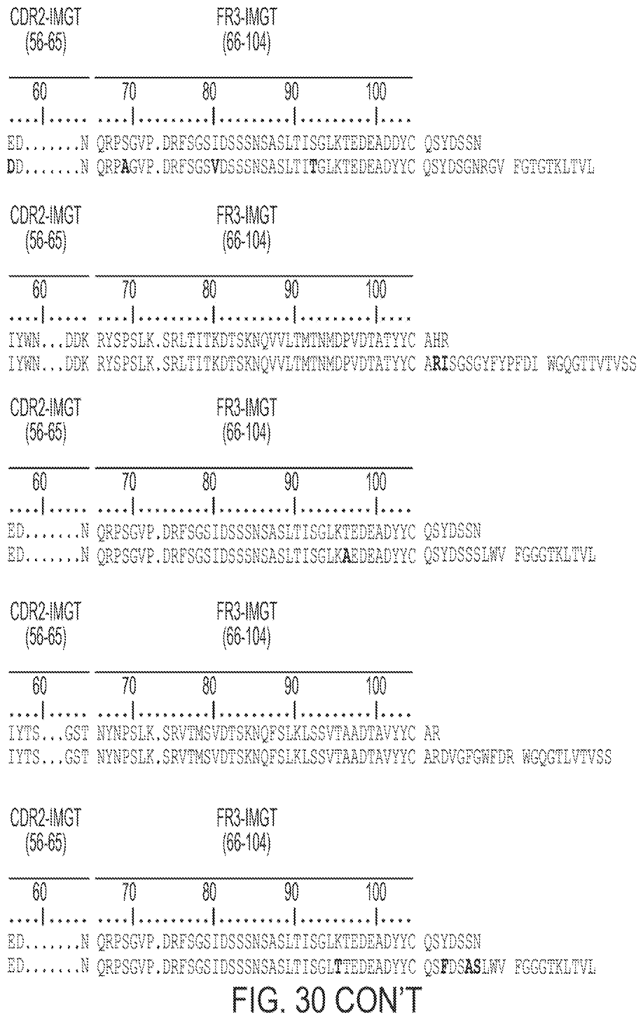

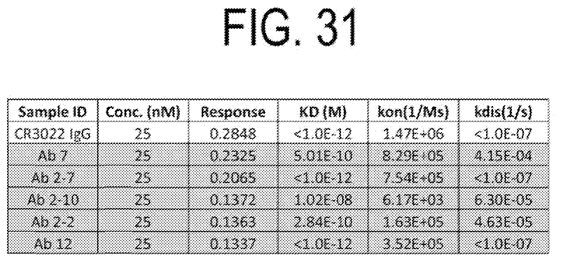

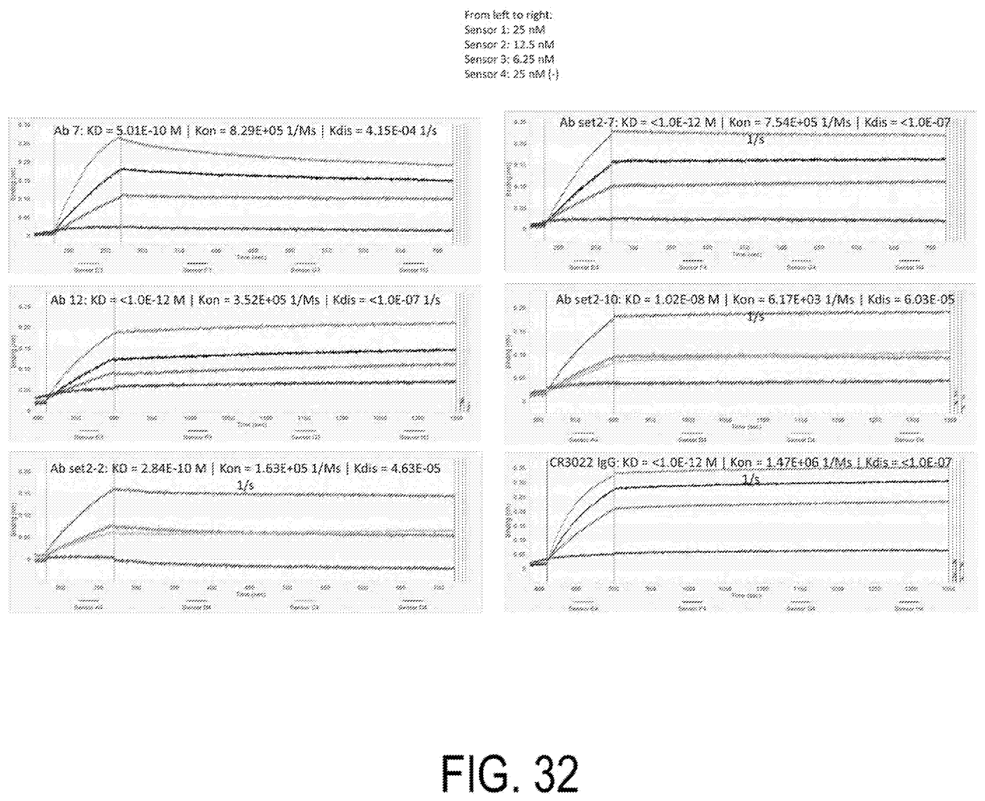

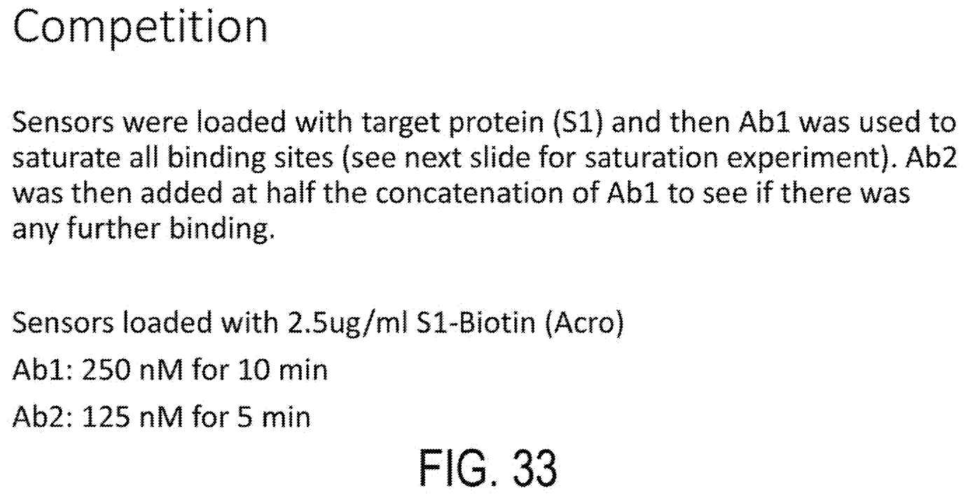

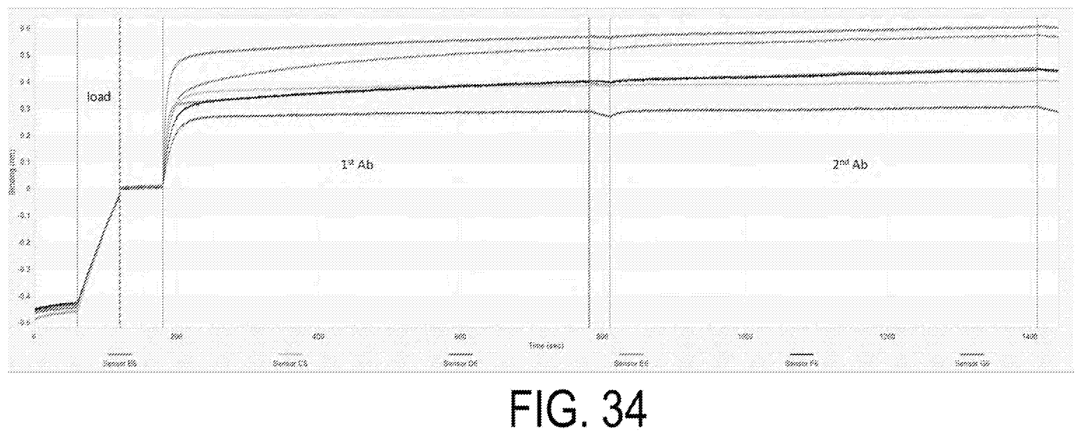

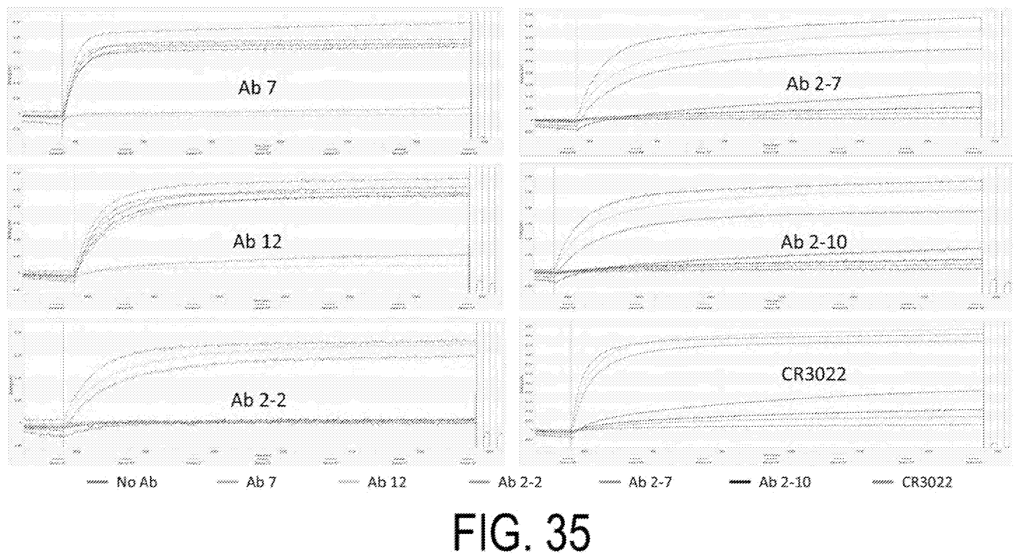

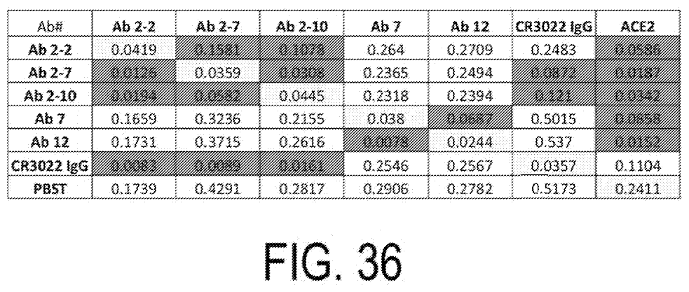

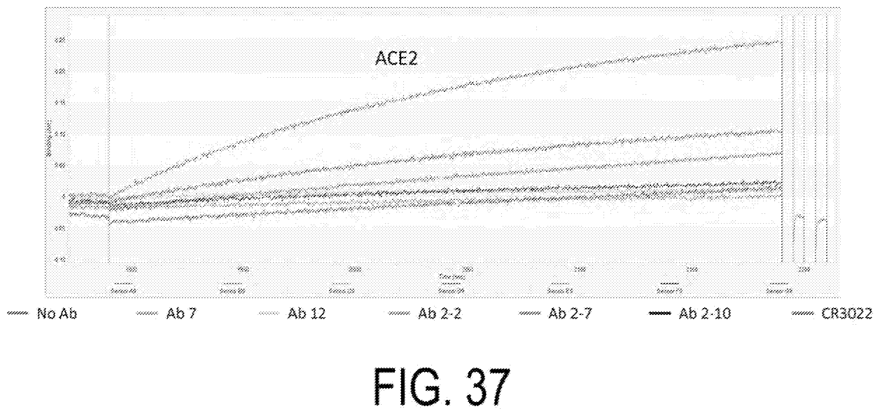

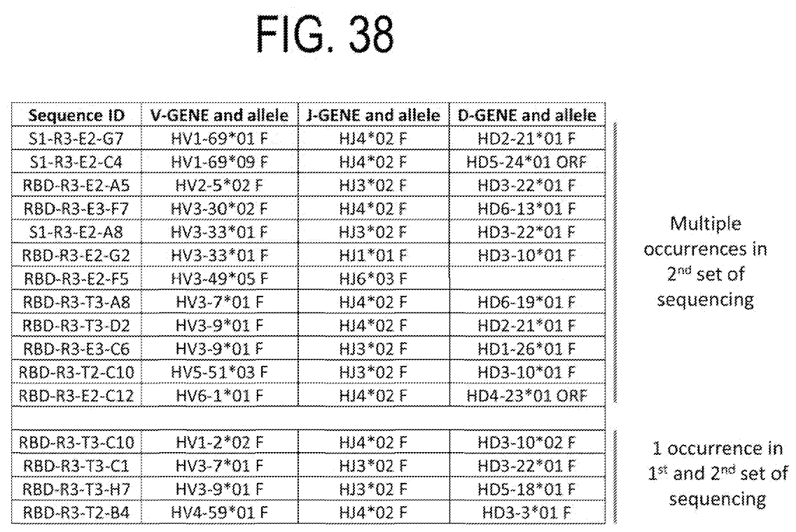

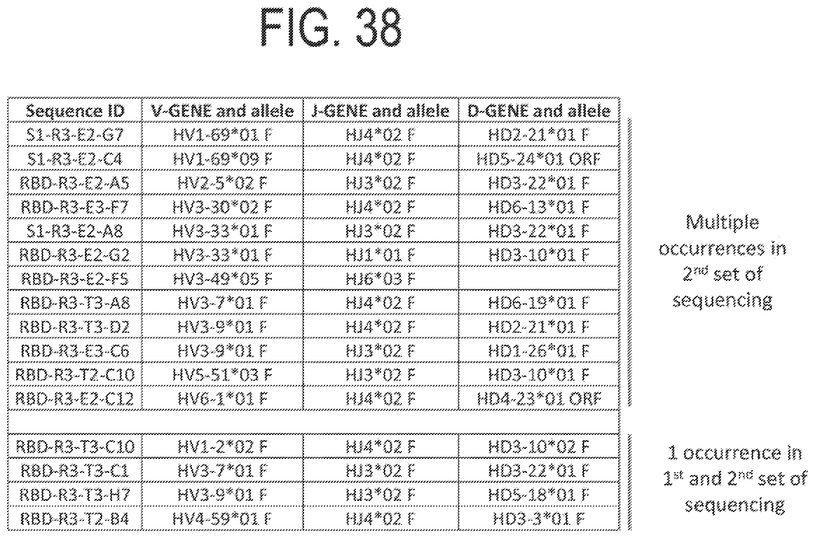

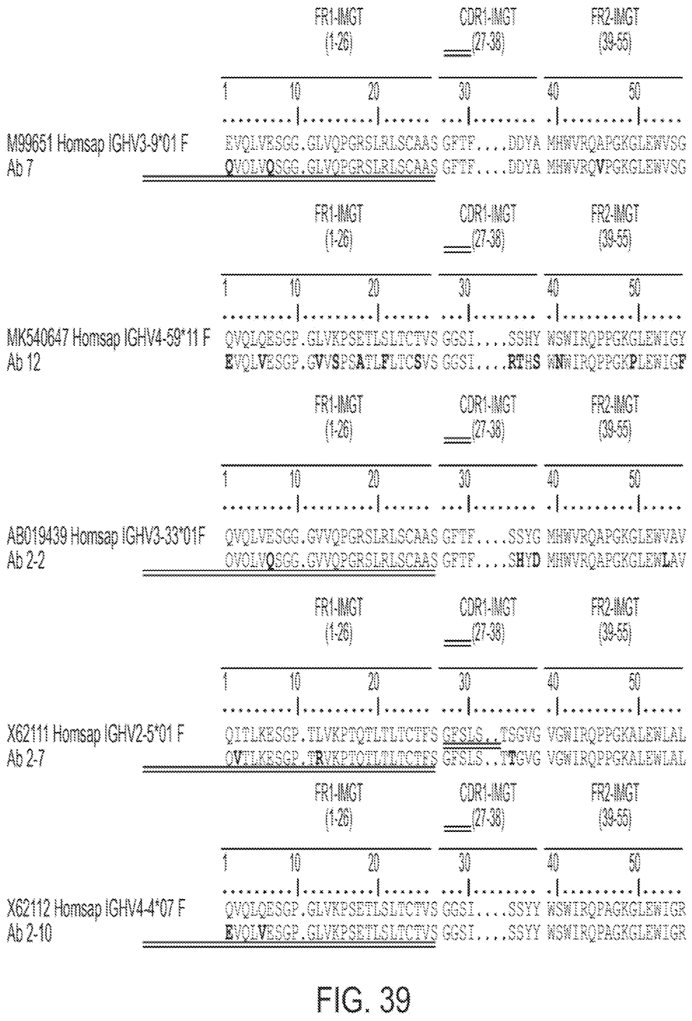

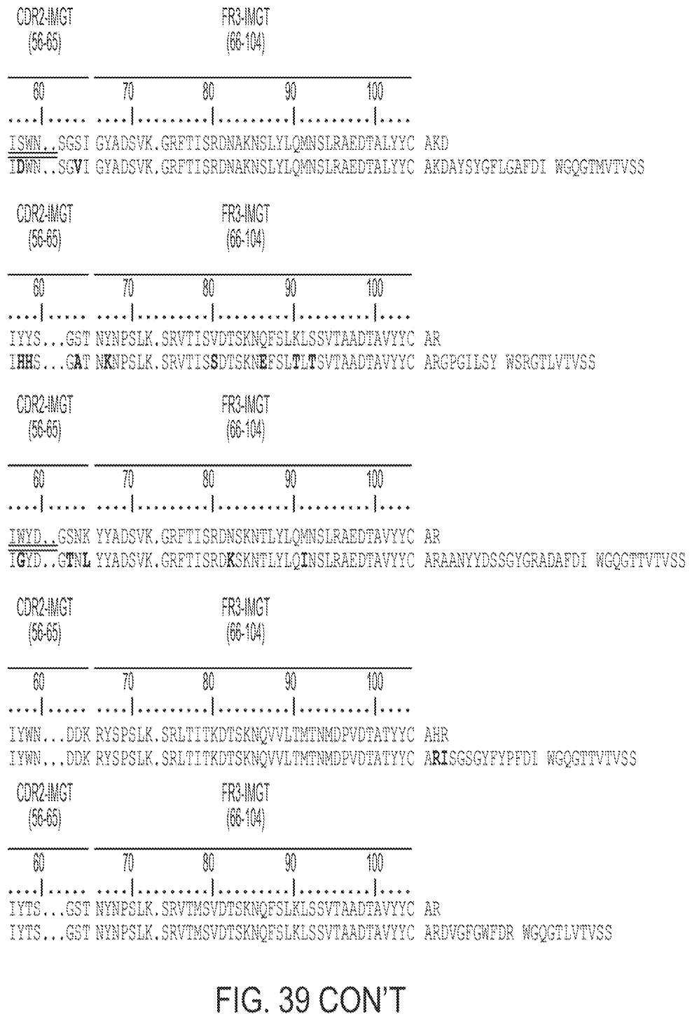

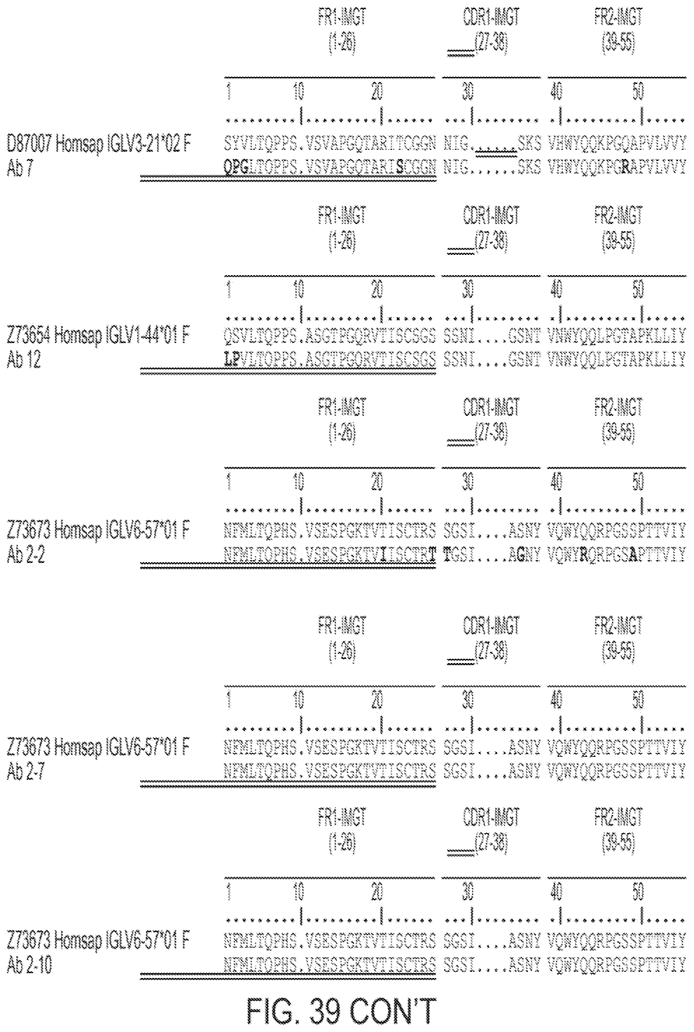

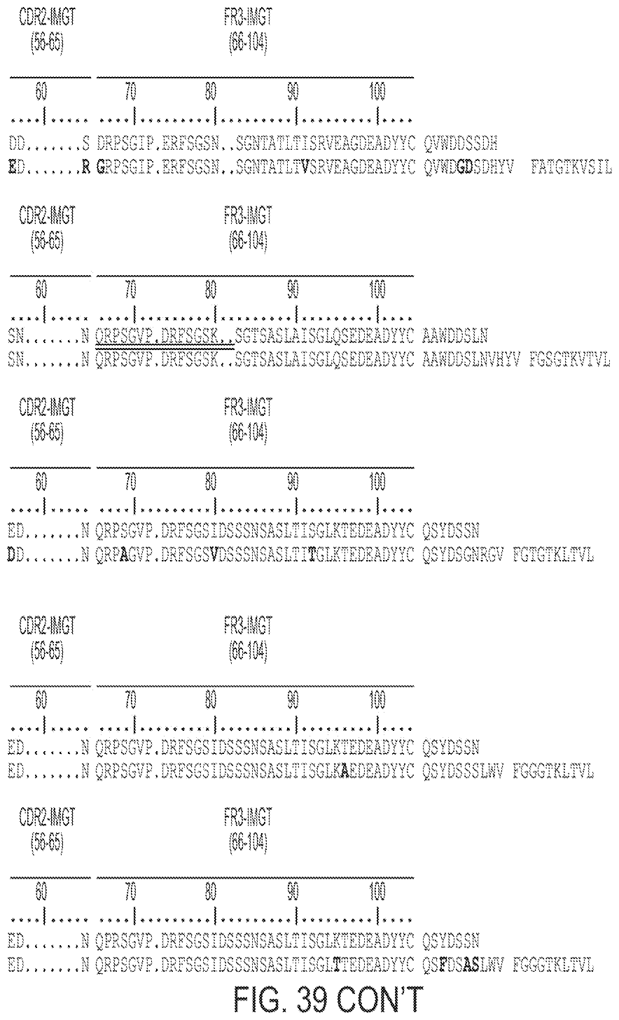

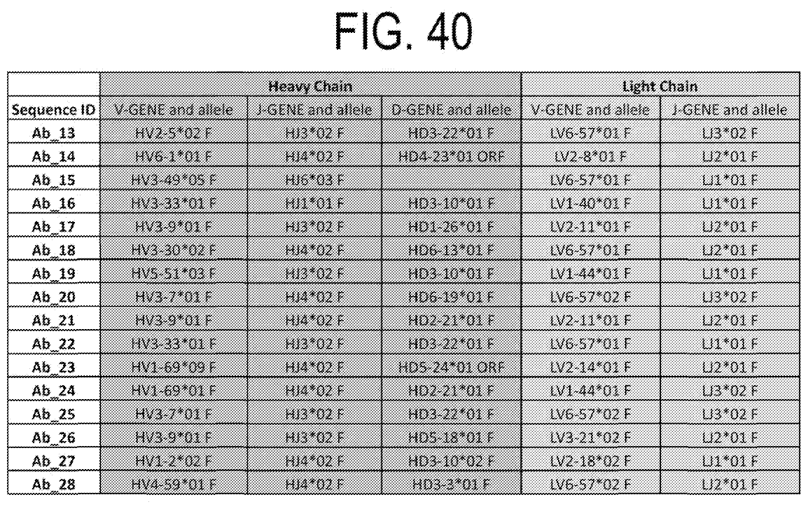

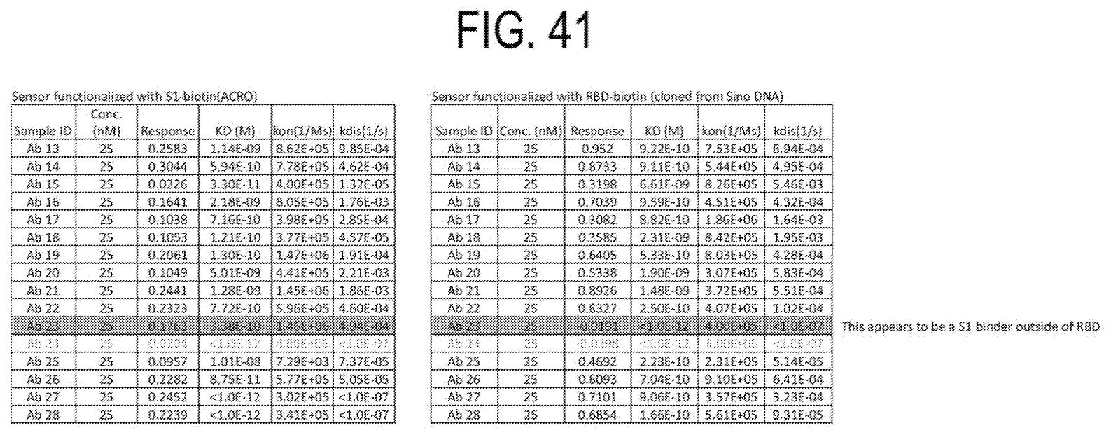

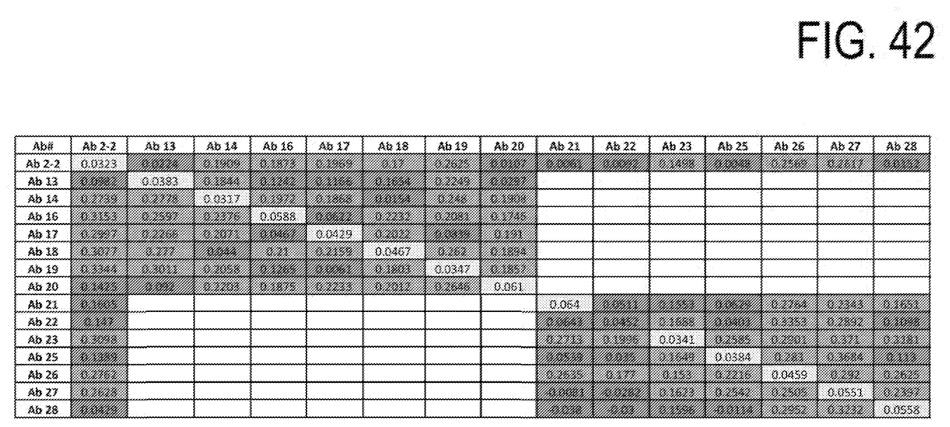

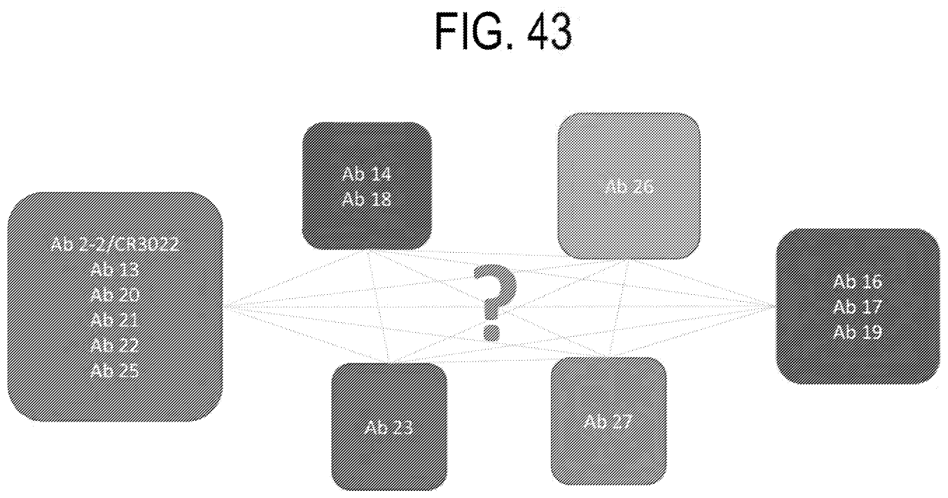

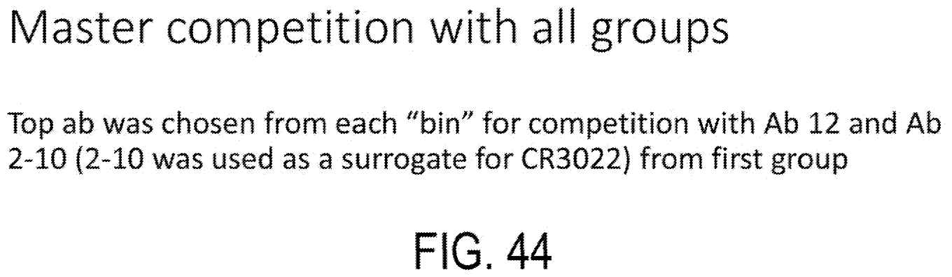

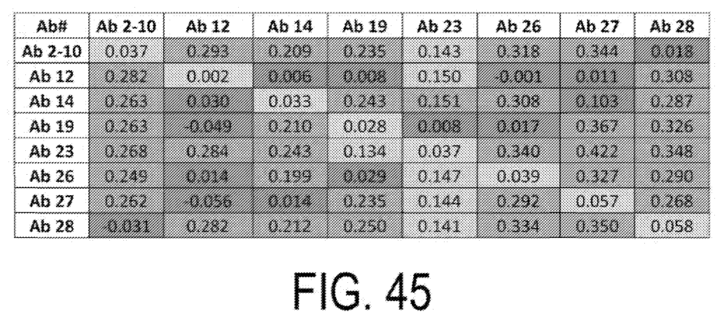

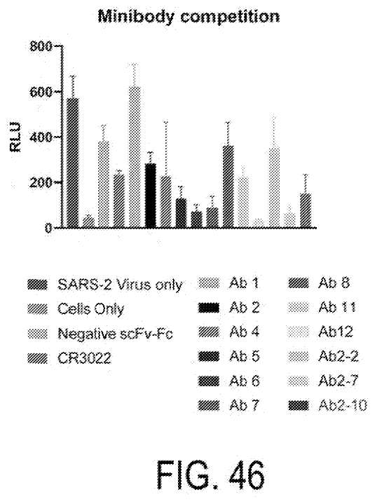

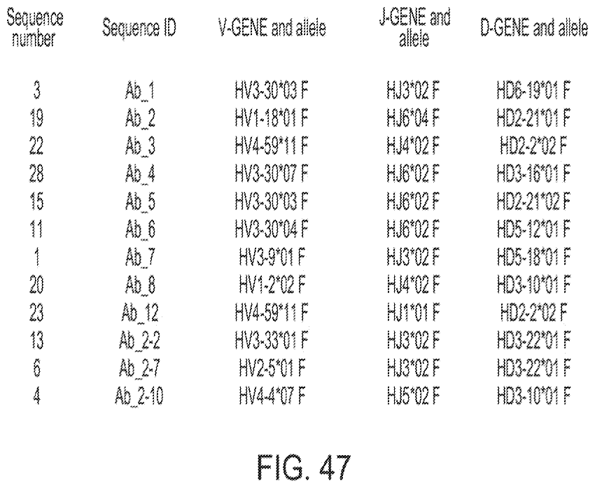

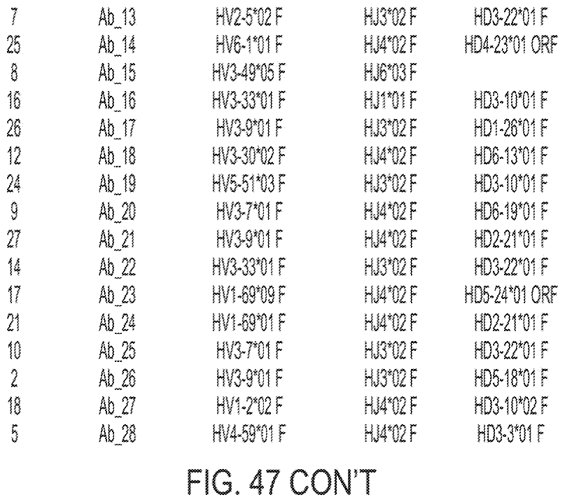

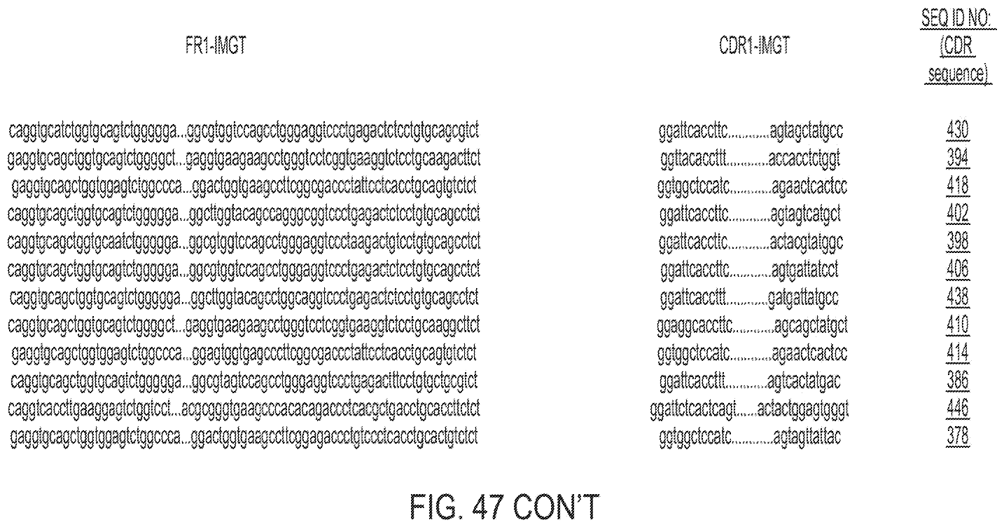

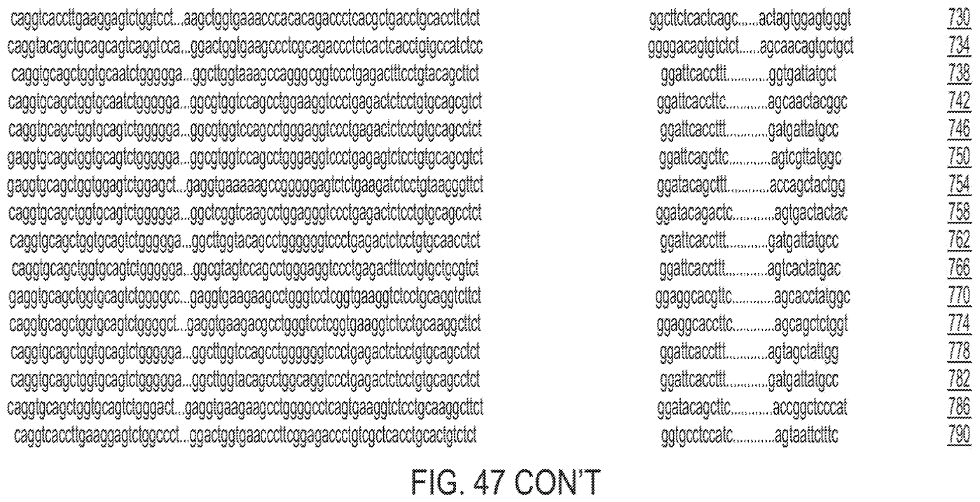

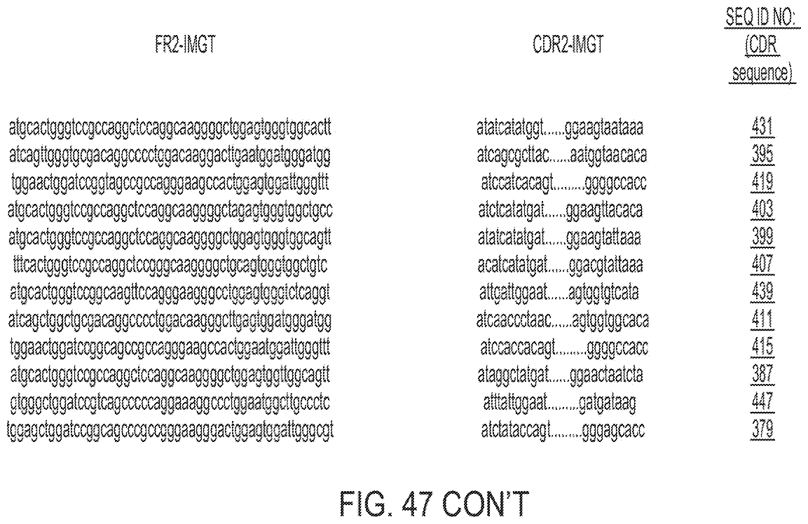

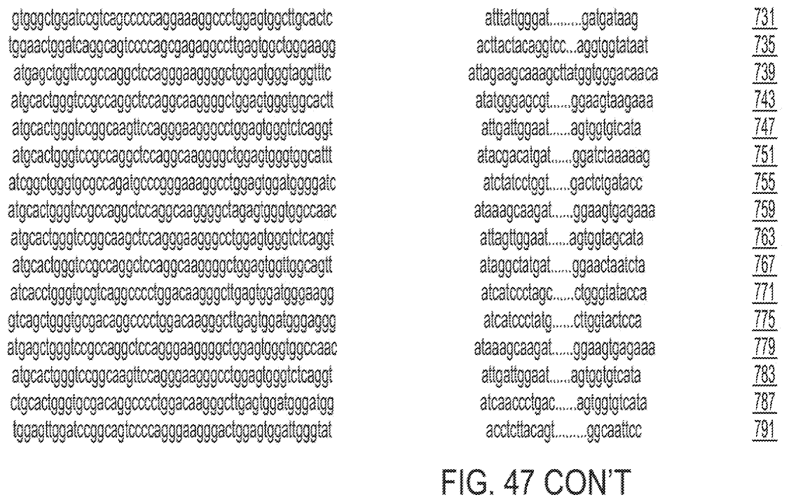

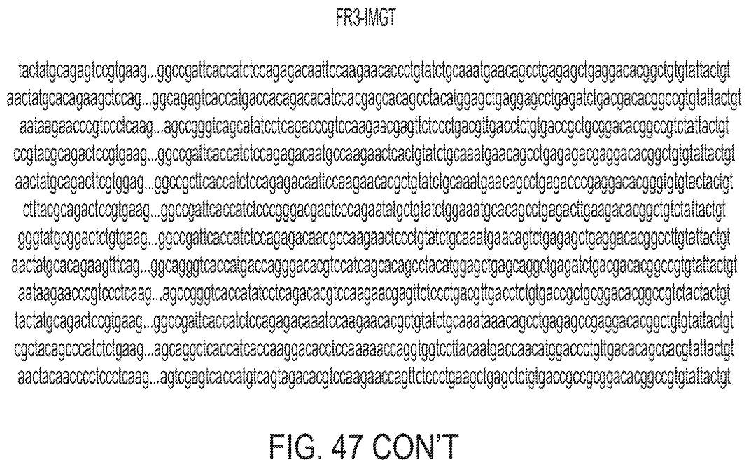

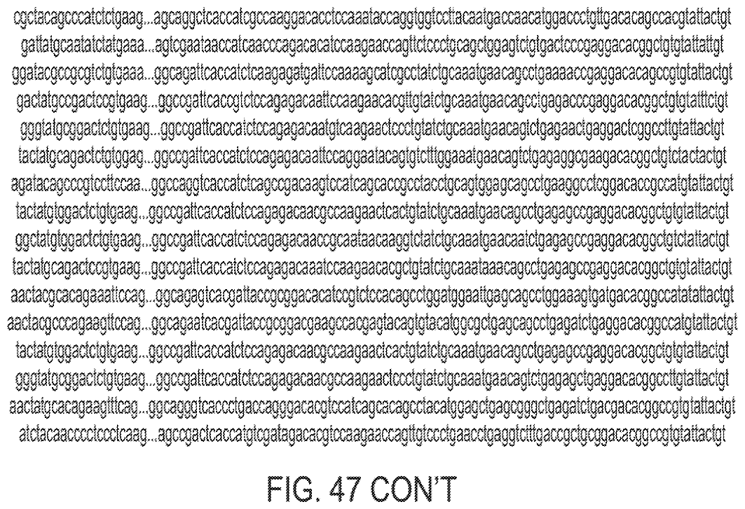

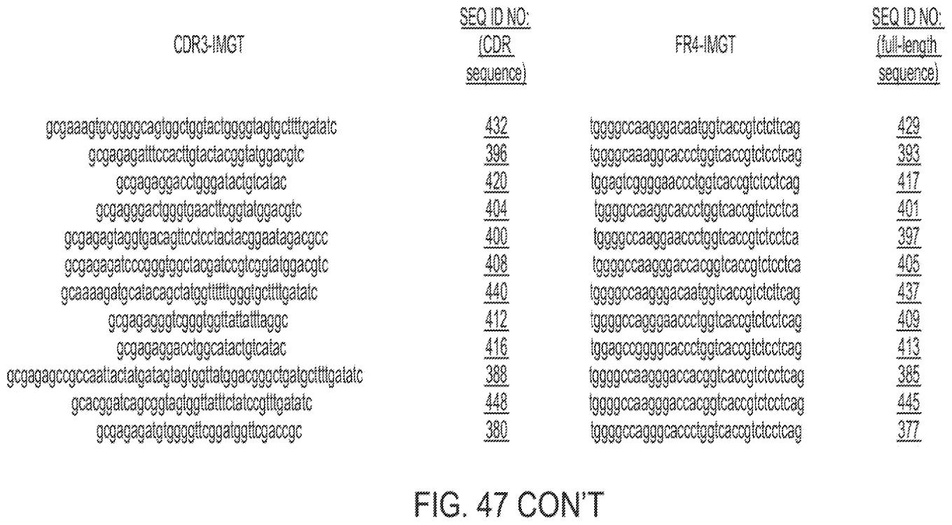

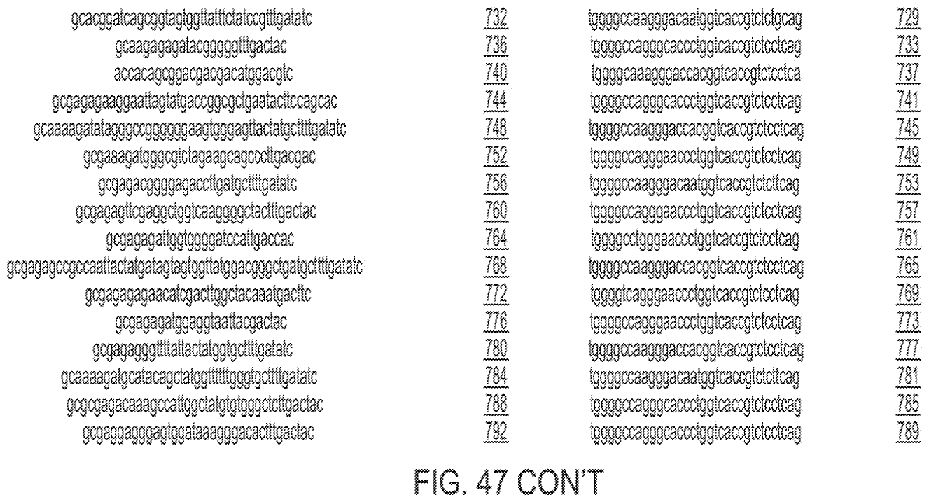

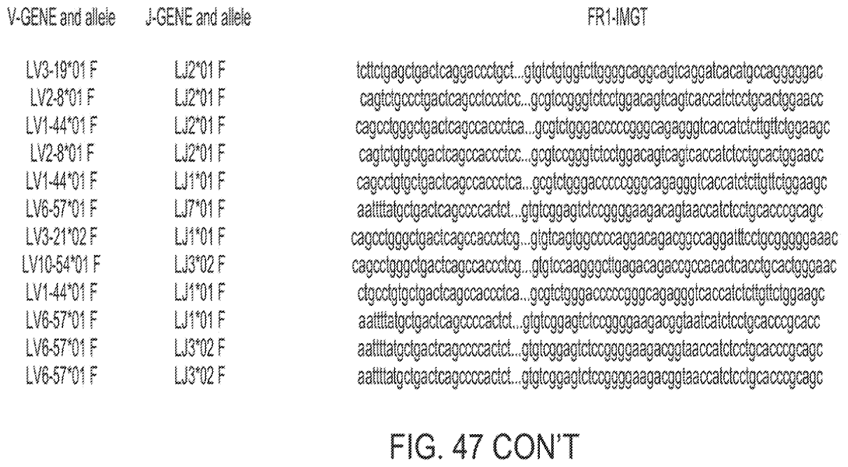

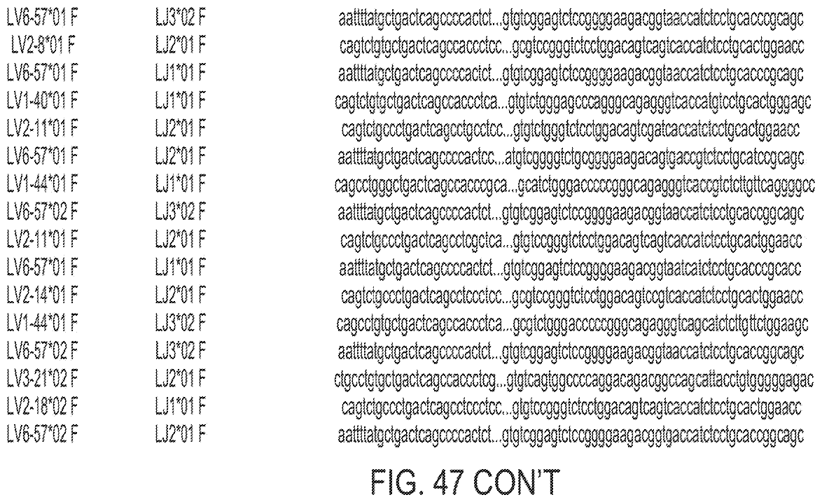

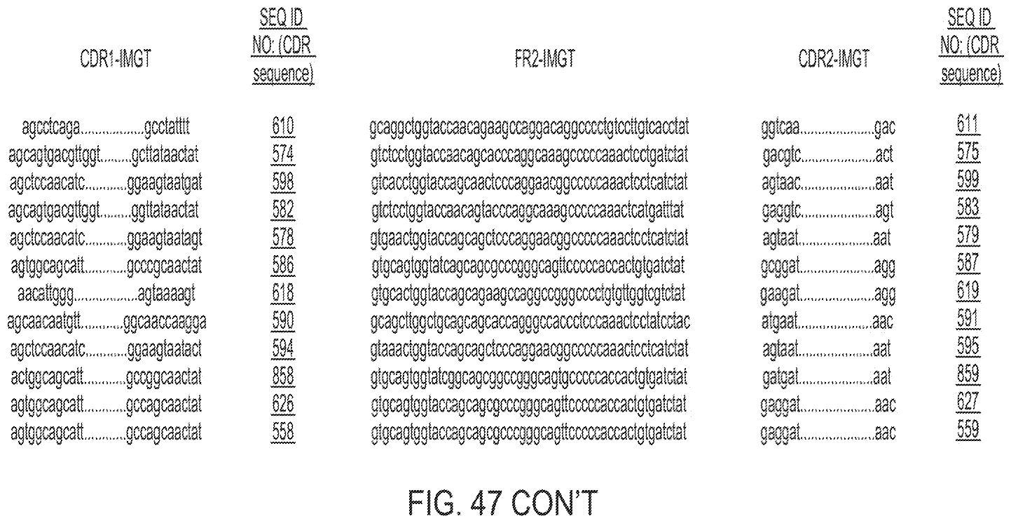

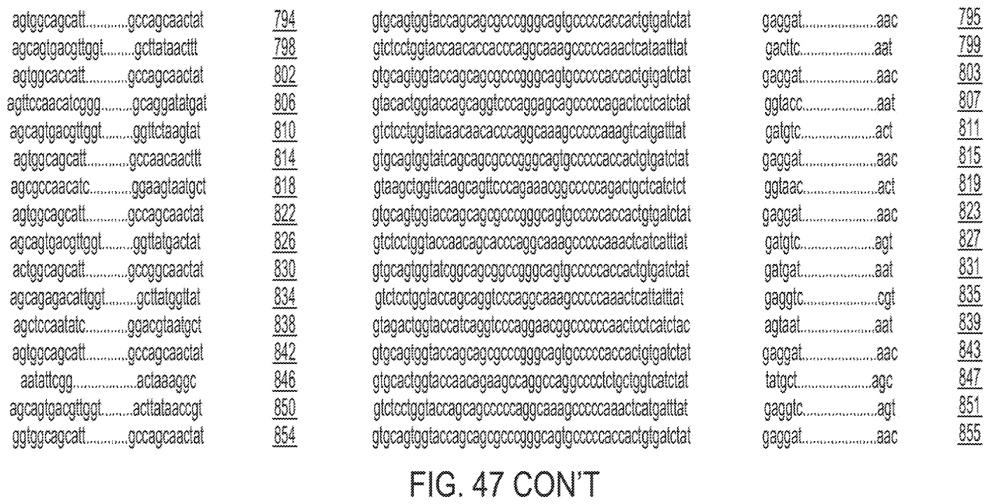

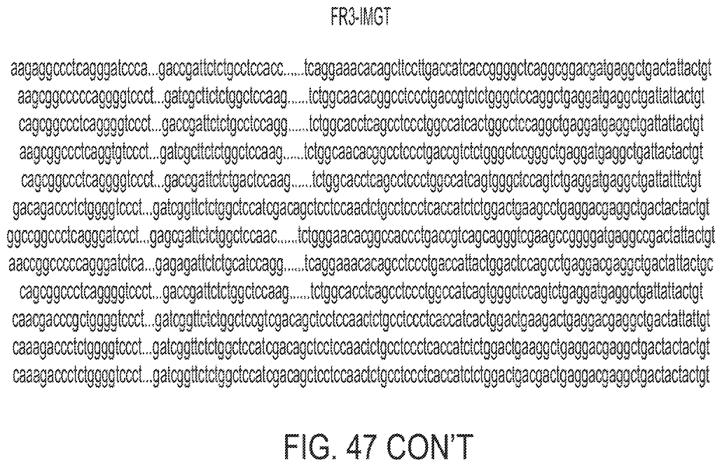

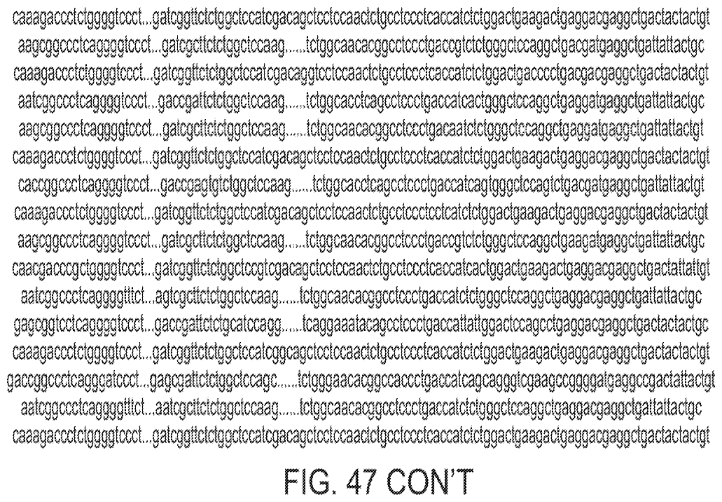

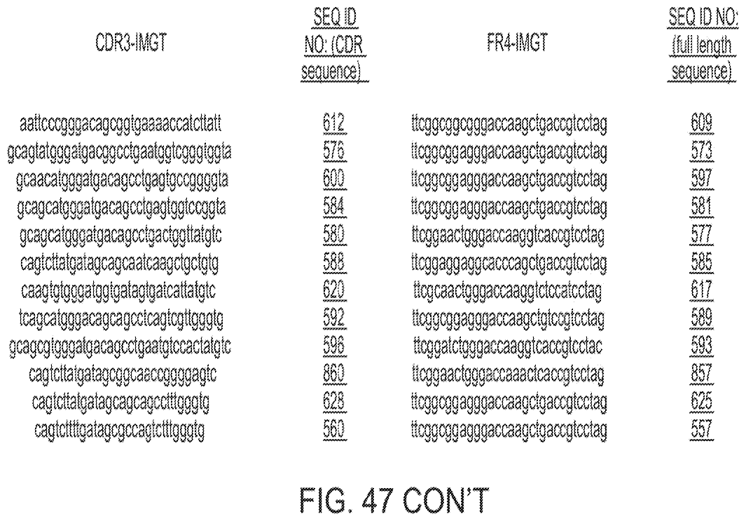

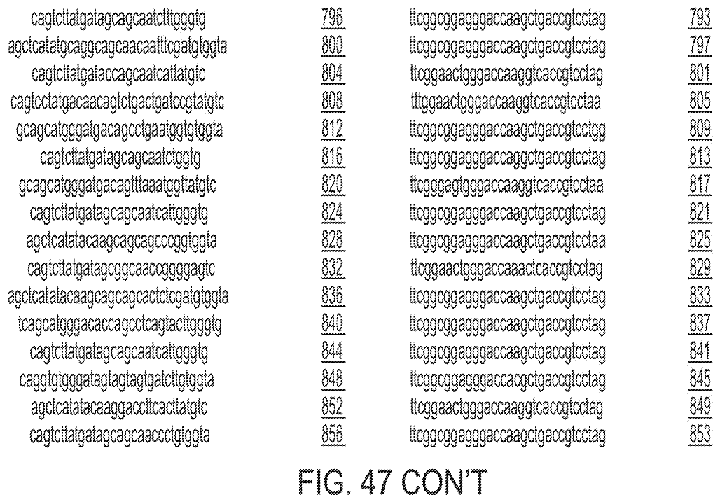

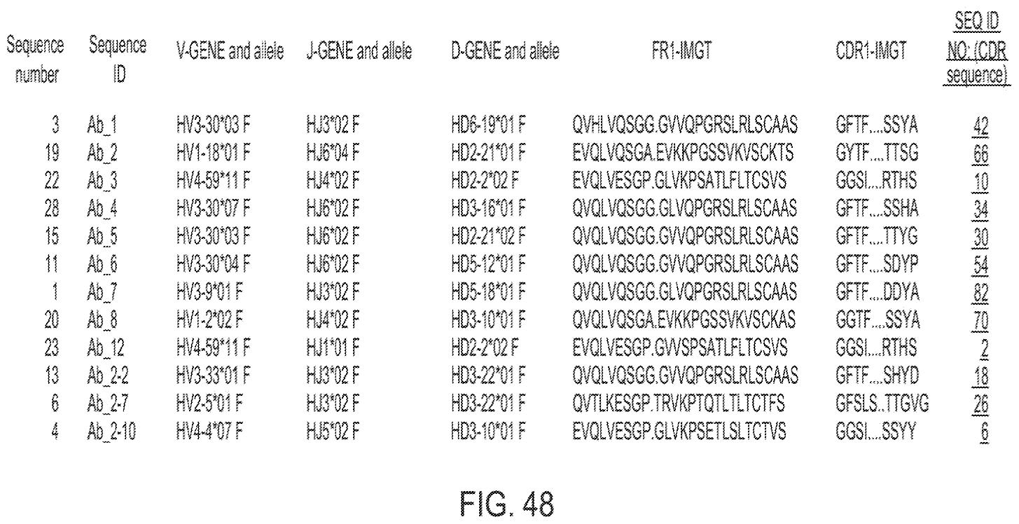

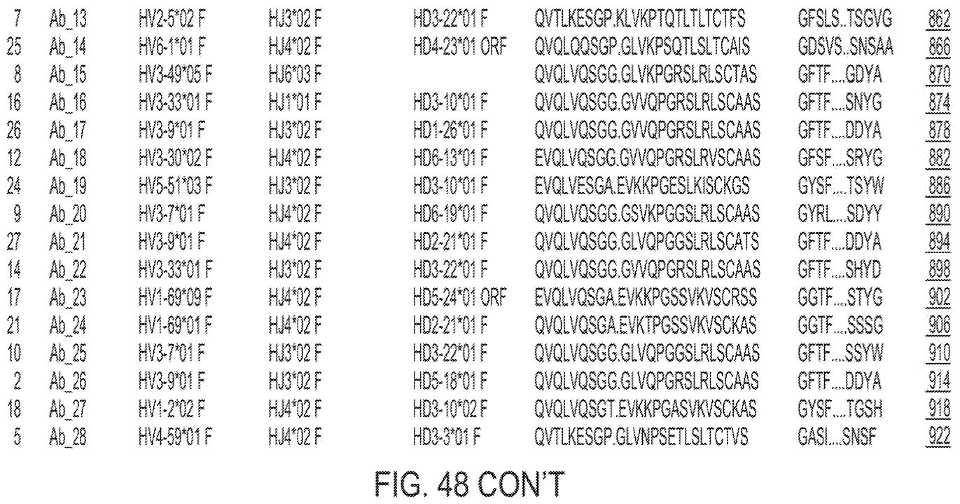

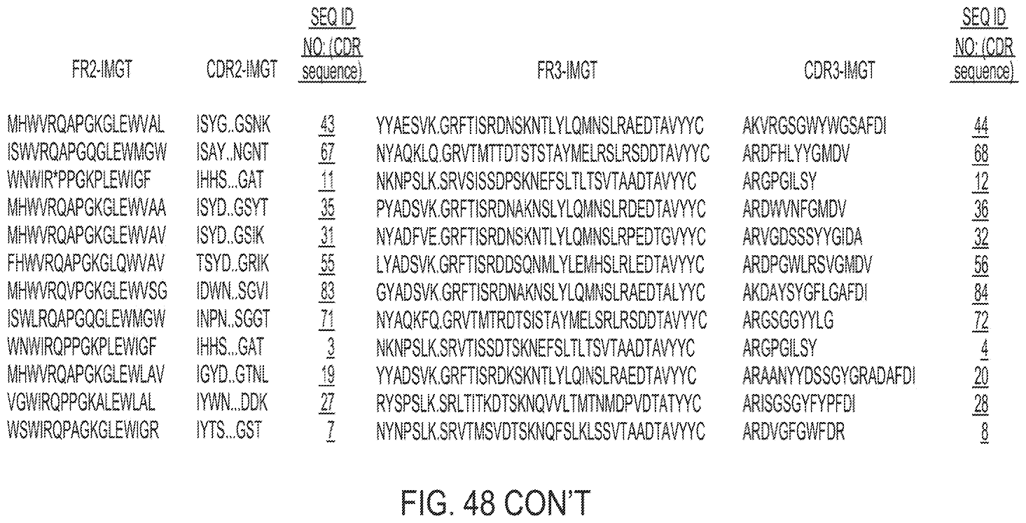

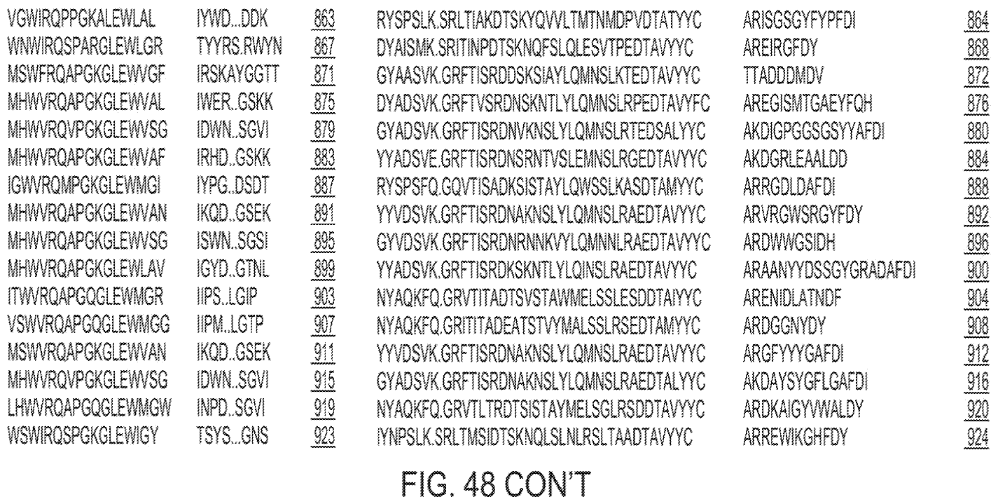

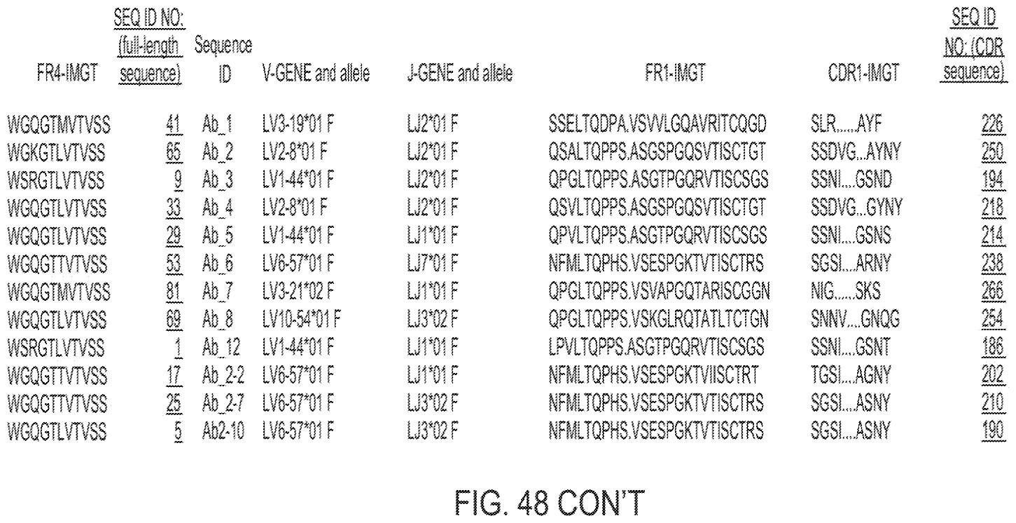

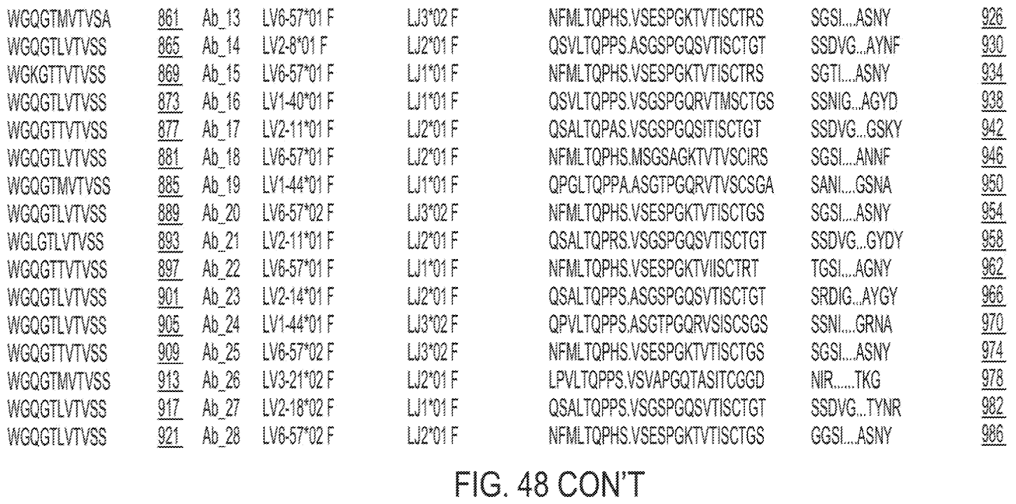

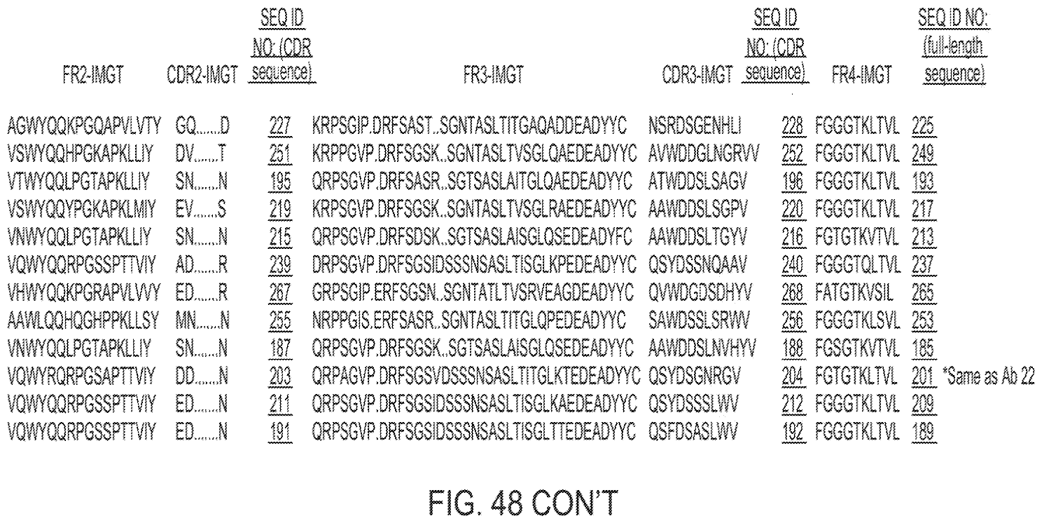

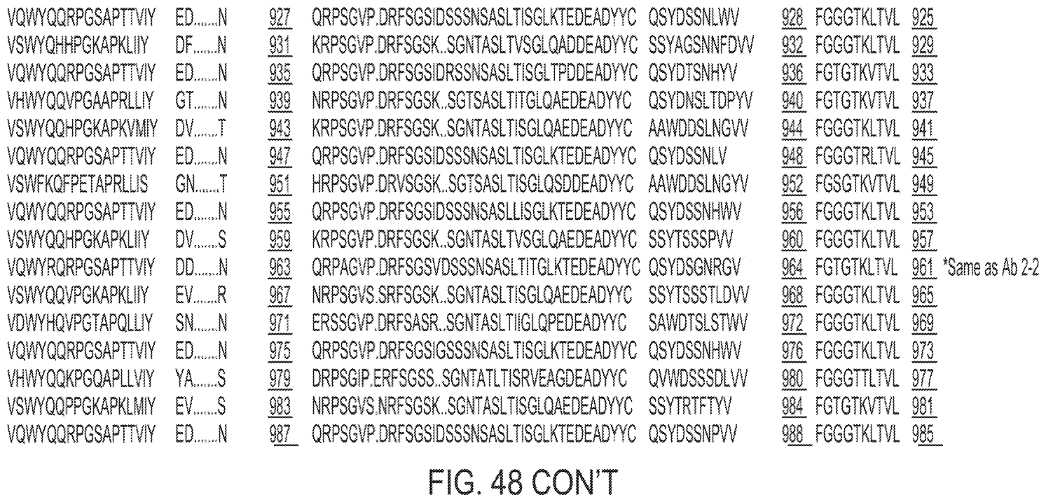

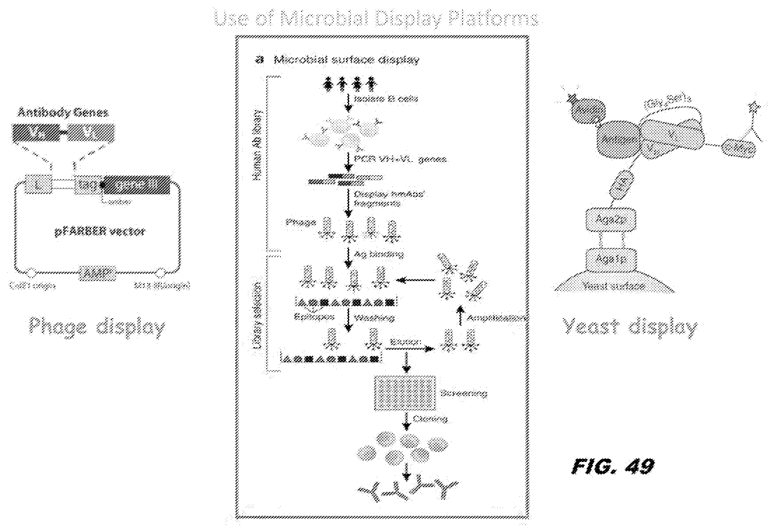

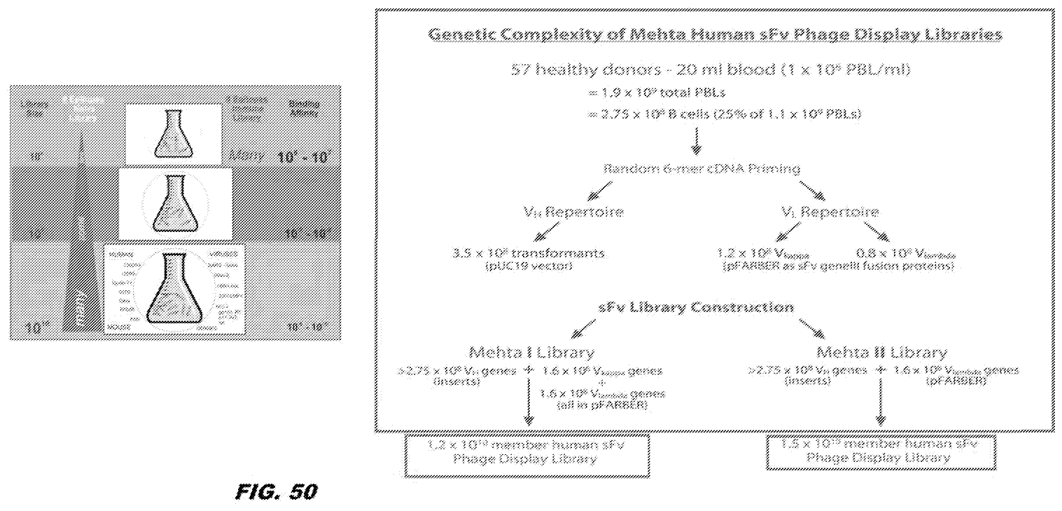

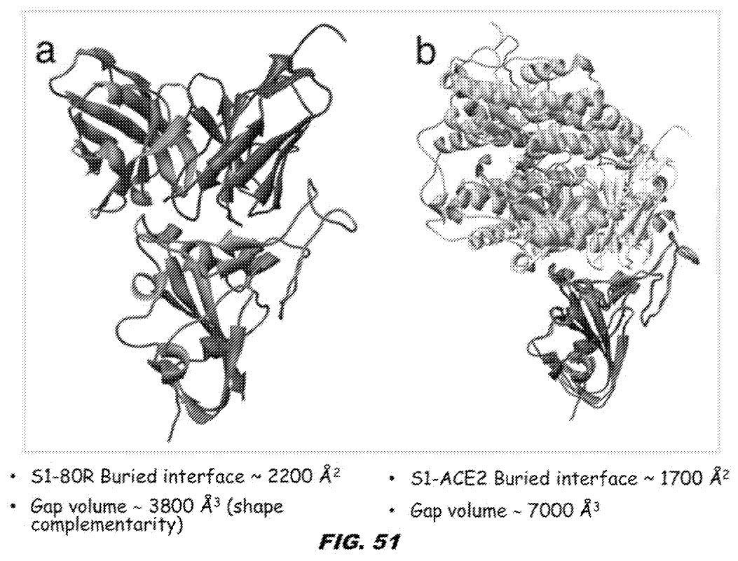

Referring to FIG. 10, for example, plate coated with RBD-Fc (0.5 μg/ml, 100 μl)

Step 1: phage added at 5E11 particles/ml, except RBD-E1-B3 was at 1E12 to move to shoulder of binding curve

Step 2: ACE2-his was added in 2× serial dilutions starting at 2 μg/ml

Step 3: phage binding was detected by anti M13-HRP; (ACE2 curve is detected via anti-his-hrp, no phage added)

Without wishing to be bound by theory, S1-RBD-T1-B12 was used as a negative control as it cannot block RBD-ACE2 binding. The anti-M13 signal is flat here, showing that ACE2 did not have any effect on phage binding.

E1-B3 and T1-F7 show distinct decreases in phage binding at higher concentrations of ACE2 indicating that there is competition for the binding site. T1-F4 shows a small decrease in signal at higher concentrations but is not as clear.

### Example 3—Kinetic Analysis of Selected scFv-Fc Candidates

FIG. 53 shows result from SARS-2 S1/RBD panning. Three rounds of panning for anti-SARS-2 S1/RBD antibodies was done using recombinantly expressed soluble protein resulting in a large number of antibodies with varying kinetic properties. The concentration of the coating protein was decreased with each round to increase the affinity of the antibodies. Two campaigns were straight panning with the three rounds against the same target protein (with different purification tags). The third campaign started with two rounds against S1, followed by a 3rd panning against the RBD protein to enrich for antibodies against the RBD. Screening was performed by picking 1344 colonies and culturing them in 2×YT media. The phage supernatants were then tested via ELISA against RBD-Fc protein (the S1 panning was also screened against S1). From our screens, >90% of the selected colonies were positive for binding to S1 or RBD. Sequencing of the positive samples yielded 73 unique clones. Kinetic analysis was performed via BLI. Octet sensors were coated with low density of biotinylated S1 protein to minimize scFv-Fc cross walking. Antibodies with low levels of binding here were found to bind biotinylated RBD coated sensors significantly better. Without wishing to be bound by theory, this can be due to the size difference of S1 versus RBD, the RBD coated sensors have a larger number of RBD molecules available for binding. Additionally, the large size of the S1 protein forces the binding even further from the sensor surface which also contributes to lower signal.

Pseudovirus neutralization was performed with SARS-2 spike pseudovirus and 293T-ACE2 transduced cells. Kinetic data for both of these abs reveal tight binding antibodies with minimal disassociation. Additionally, epitope mapping reveals that they have similar but slightly different competition patterns (FIG. 54). Ab 12 successfully competes with Abs 14, 15, 19, 26, and 27. While Ab 27 also competes with Abs 12, 14, and 15, it does not compete with Ab 19 or Ab 26. Without wishing to be bound by theory, the antibodies bind similar epitopes but have a different angle of approach.

### Example 4—Neutralization Studies

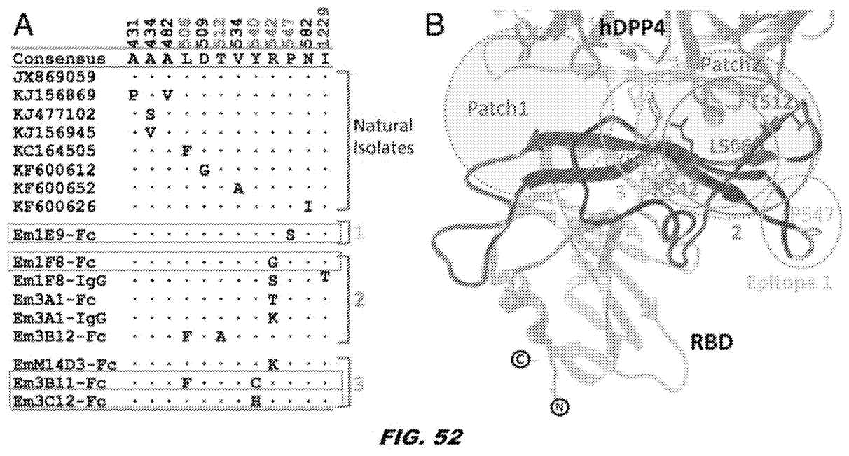

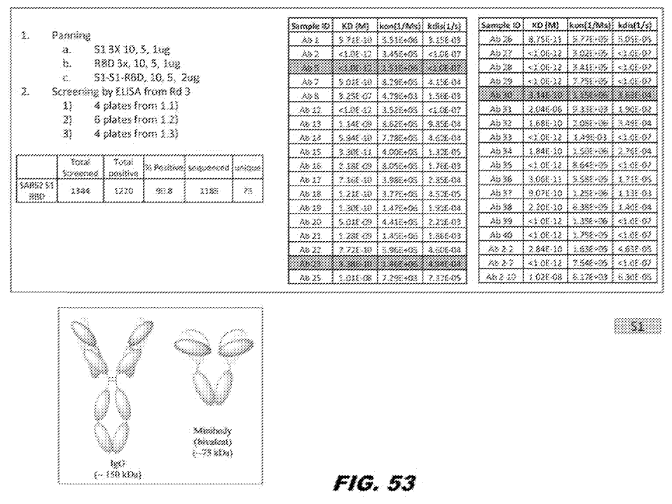

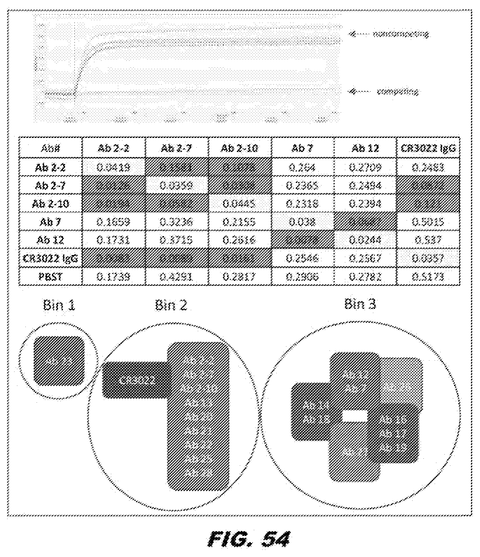

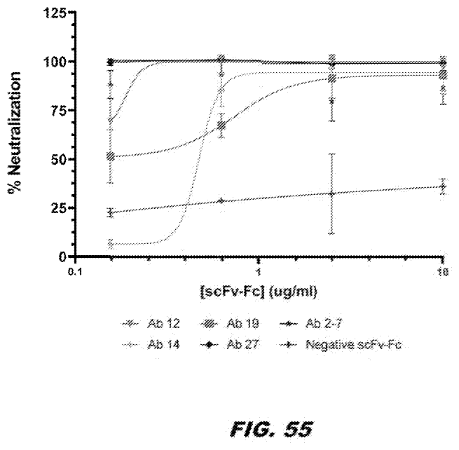

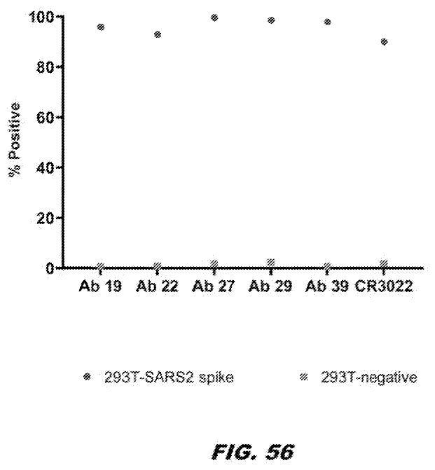

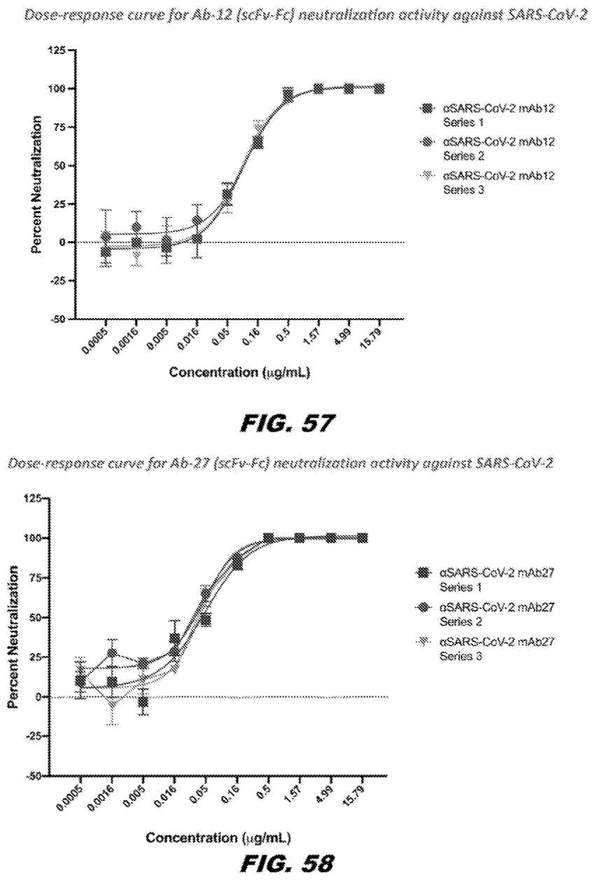

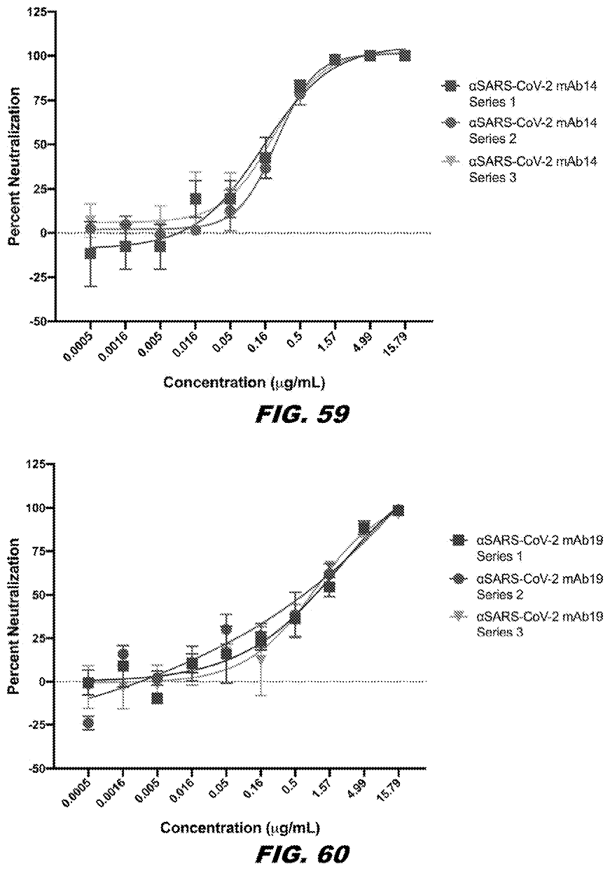

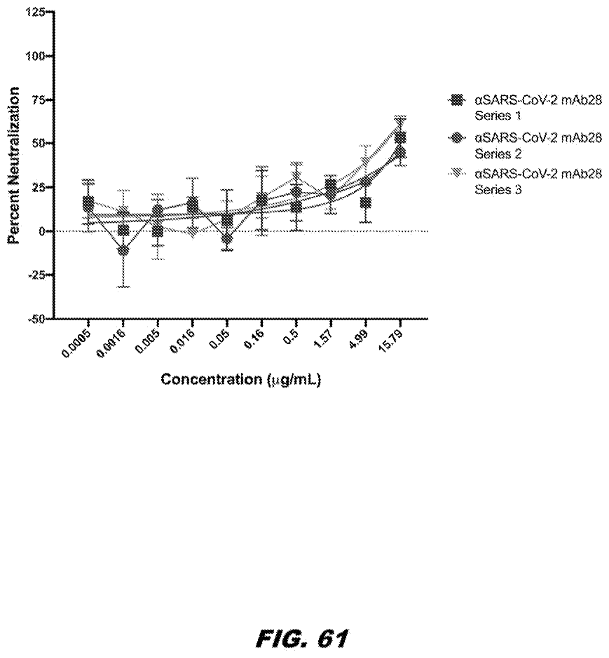

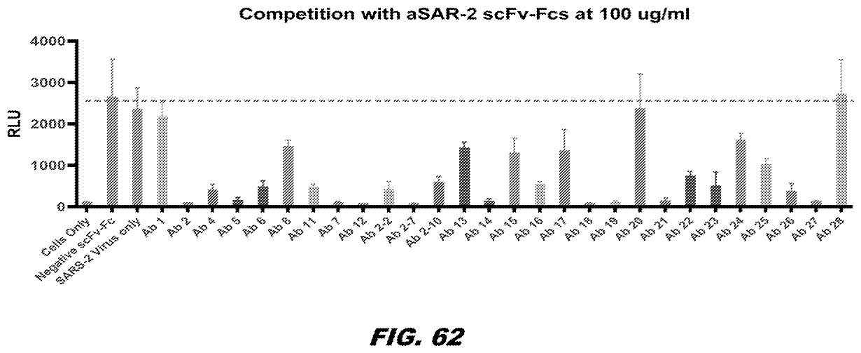

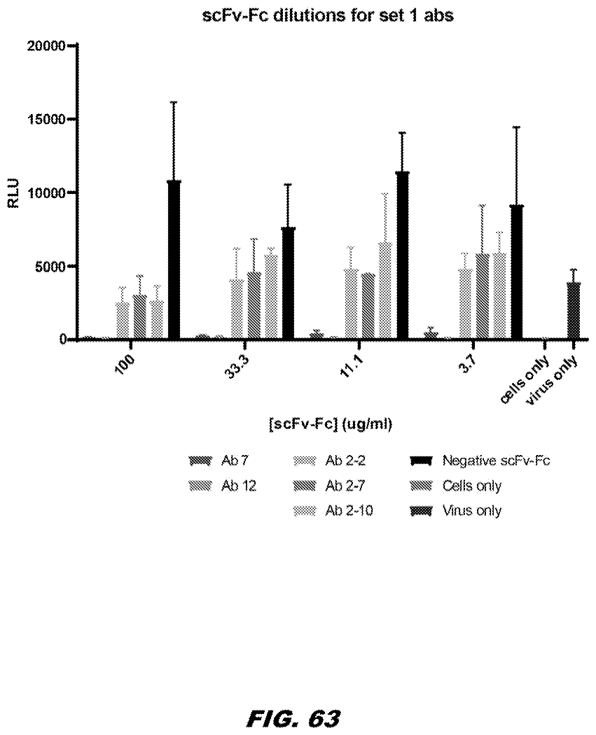

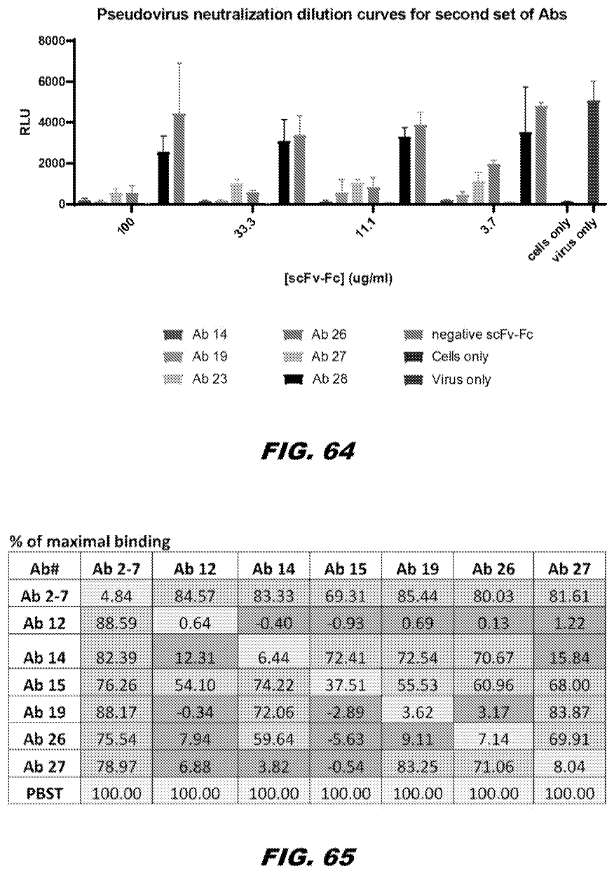

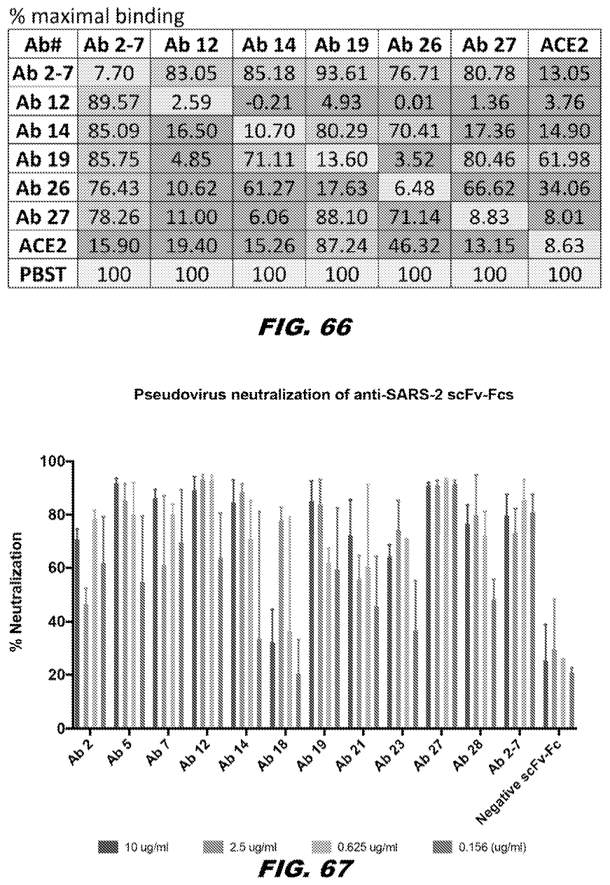

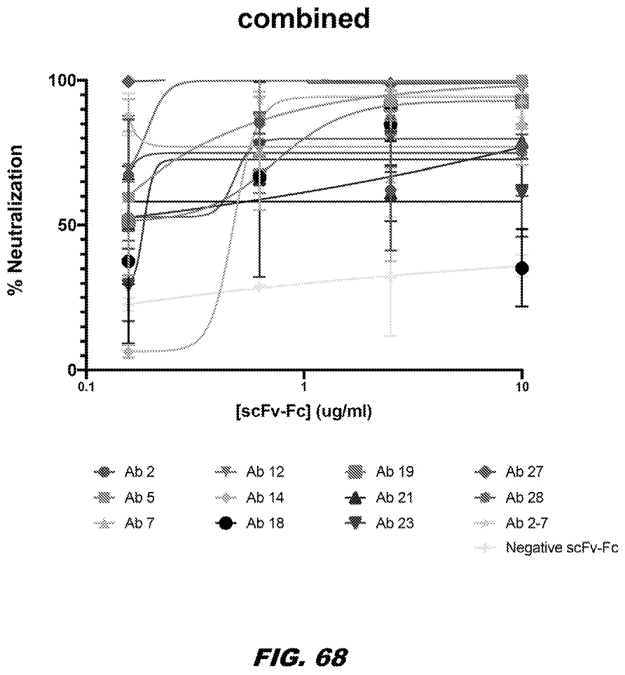

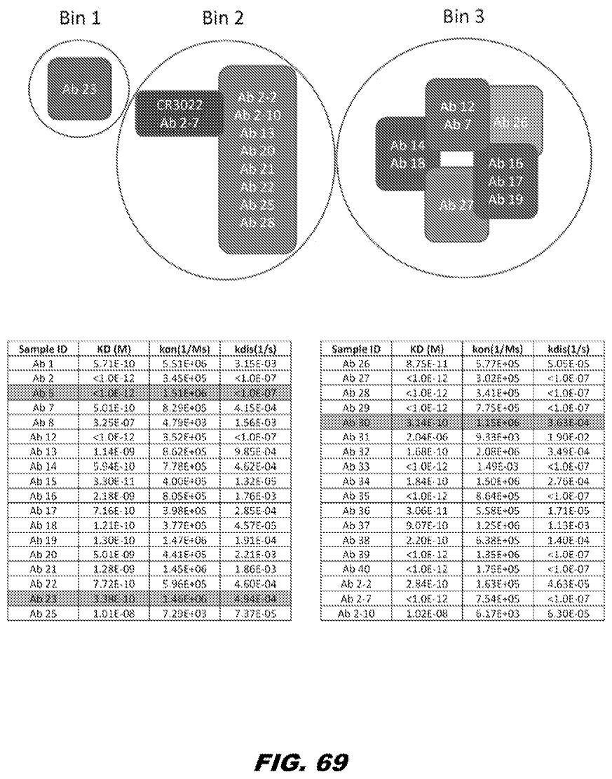

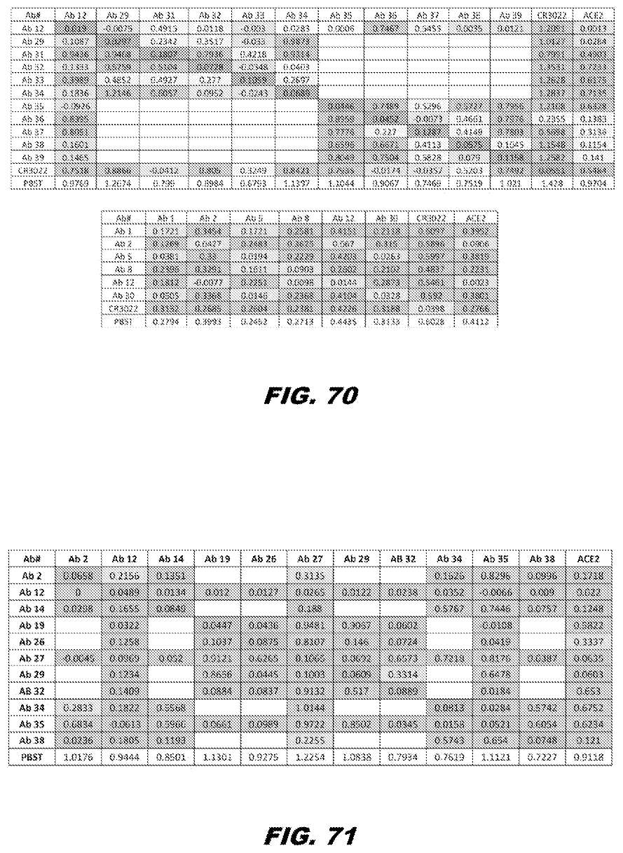

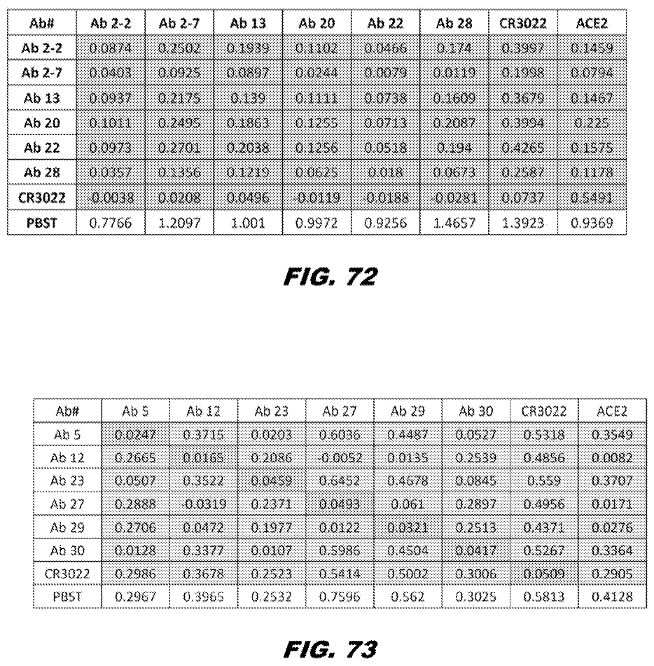

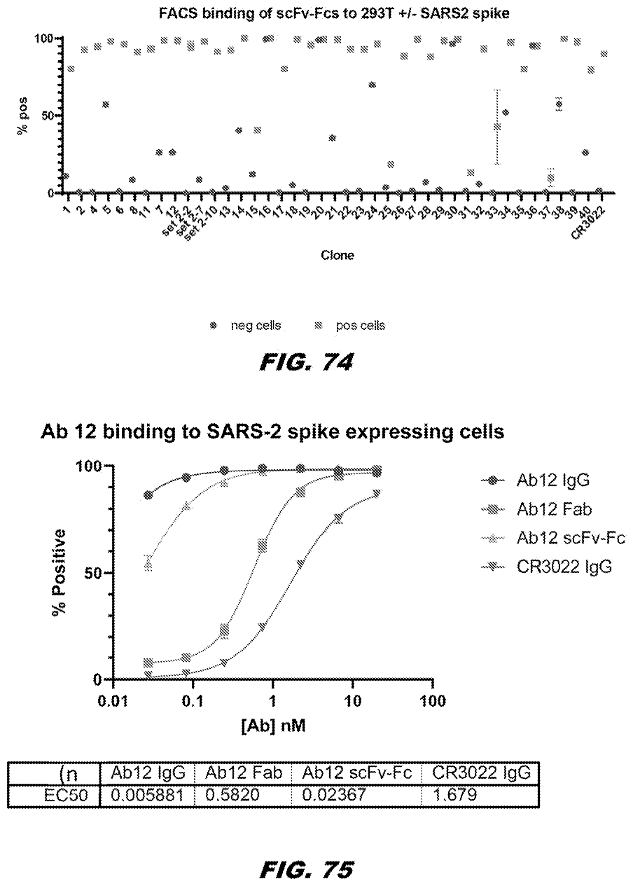

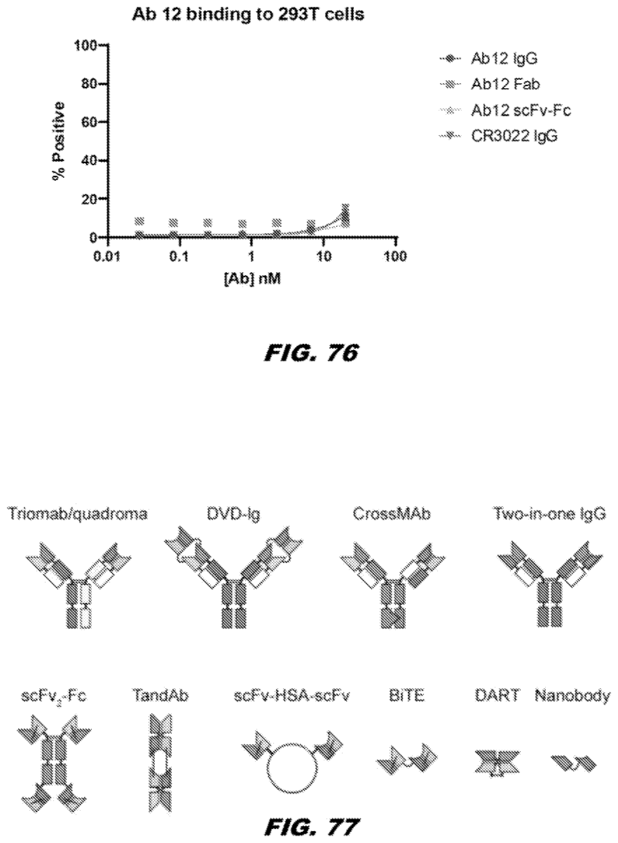

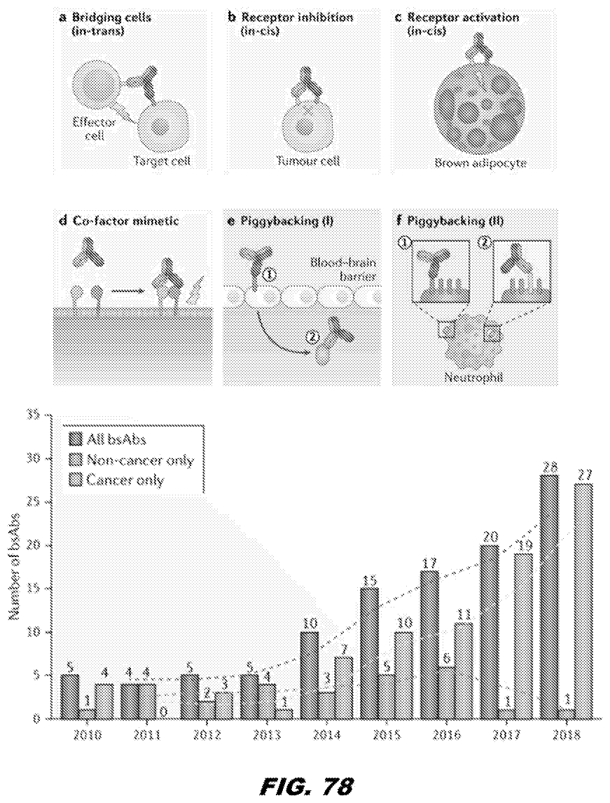

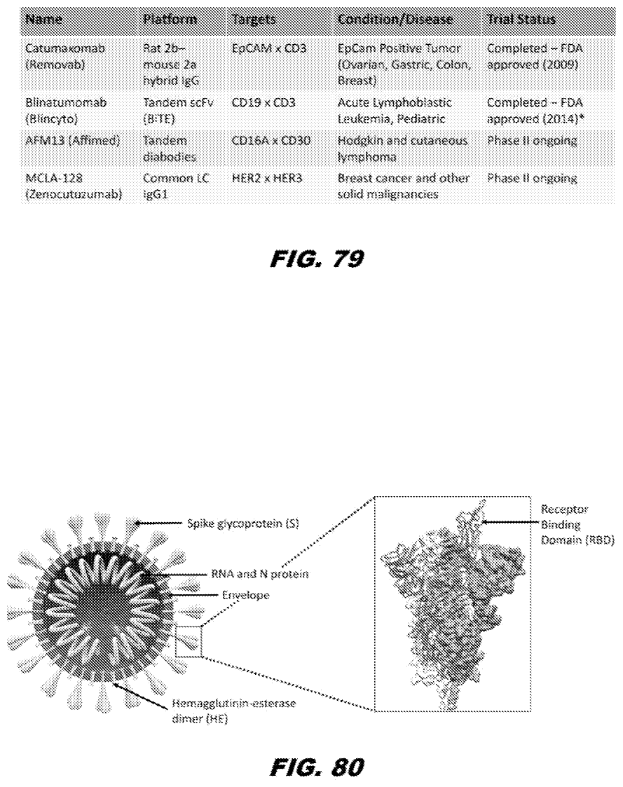

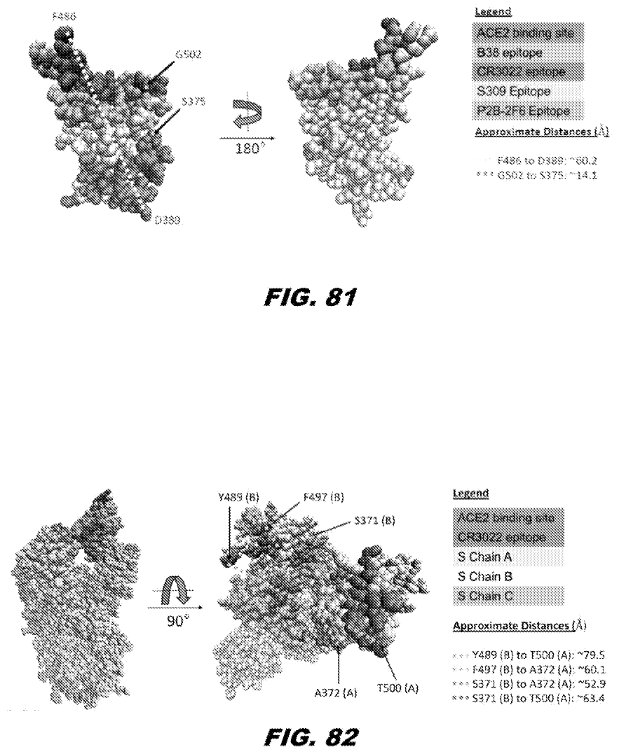

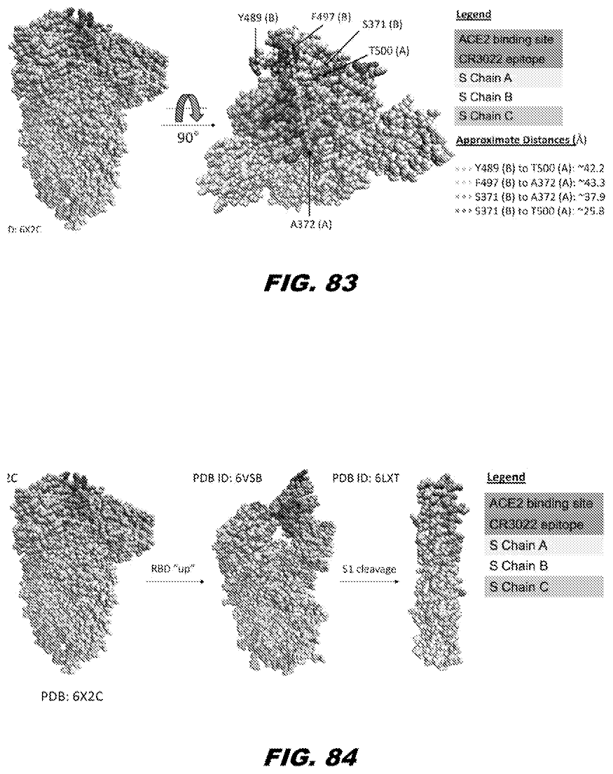

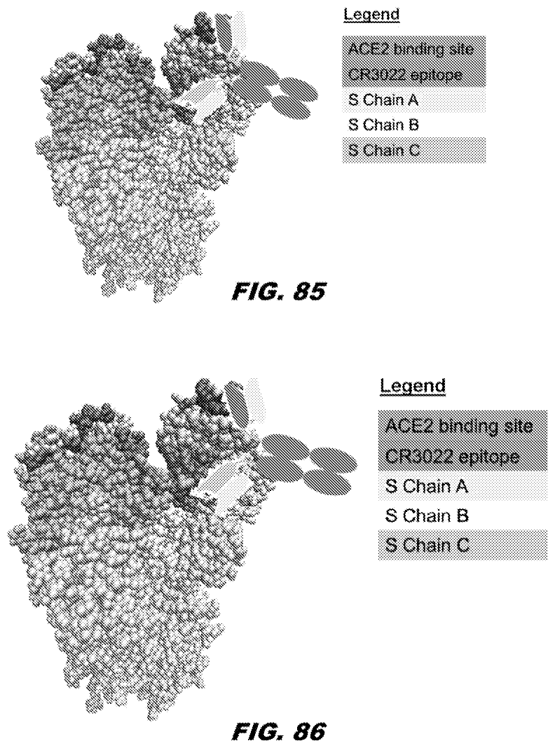

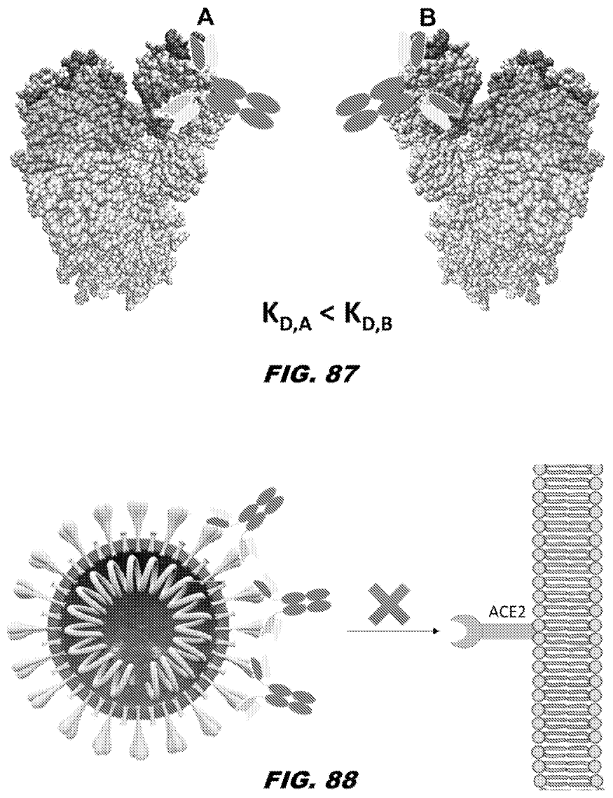

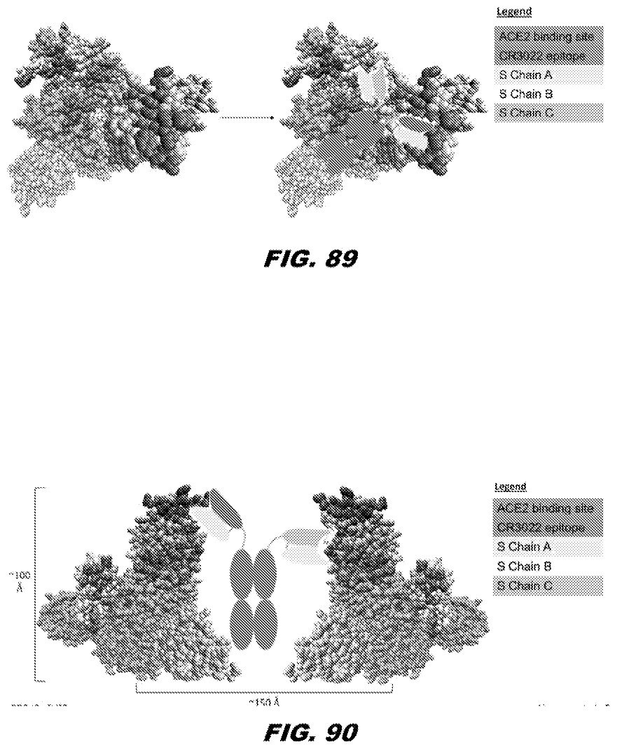

Pseudovirus neutralization was performed with SARS-2 spike pseudovirus and 293T-ACE2 transduced cells. As shown in FIG. 55, a number of neutralizing antibodies were identified with Ab 12 and Ab 27 being the most potent.

Kinetic data for both of these abs reveal tight binding antibodies with minimal disassociation. Additionally, epitope mapping reveals that they have similar but slightly different competition patterns. Ab 12 successfully competes with Abs 14, 15, 19, 26, and 27. While Ab 27 also competes with Abs 12, 14, and 15, it does not compete with Ab 19 or Ab 26. This indicates that they bind similar epitopes but can have a different angle of approach.

### Example 5—Live Virus Neutralization

Monoclonal antibodies were diluted to 100 ug/ml by adding 50 μl of 1 mg/ml antibody to 450 μl Dulbecco's Phosphate Buffered Saline (DPBS)(Gibco™). A series of 10 half-log dilutions was then prepared in triplicate for each antibody in DPBS. Each dilution was incubated at 37° C. and 5% CO₂ for 1 hour with 1000 plaque forming units/ml (PFU/ml) of SARS-CoV-2 (isolate USA-WA1/2020), diluted in Dulbecco's Modified Eagle Medium (DMEM) (Gibco™) containing 2% fetal bovine serum (Gibco™) and antibiotic-antimycotic (Gibco™). Controls included DMEM containing 2% fetal bovine serum and antibiotic-antimycotic only as a negative control, 1000 PFU/ml SARS-CoV-2 incubated with DPBS, and 1000 PFU/ml SARS-CoV-2 incubated with DMEM. Two hundred microliters of each dilution or control were added to confluent monolayers of NR-596 Vero E6 cells in triplicate and incubated for 1 hour at 37° C. and 5% CO₂. The plates were gently rocked every 5-10 minutes to prevent monolayer drying. The monolayers were then overlaid with a 1:1 mixture of 2.5% Avicel® RC-591 microcrystalline cellulose and carboxymethylcellulose sodium (DuPont Nutrition & Biosciences, Wilmington, DE) and 2× Modified Eagle Medium (Temin's modification, Gibco™) supplemented with 2× antibiotic-antimycotic (Gibco™), 2× GlutaMAX (Gibco™) and 10% fetal bovine serum (Gibco™). Plates were incubated at 37° C. and 5% CO² for 2 days. The monolayers were fixed with 10% neutral buffered formalin and stained with 0.2% aqueous Gentian Violet (RICCA Chemicals, Arlington, TX) in 10% neutral buffered formalin for 30 min, followed by rinsing and plaque counting. The half maximal inhibitory concentrations (IC50) were calculated using GraphPad Prism 8.

TABLE I
IC₅₀ values for monoclonal antibody neutralization
activity against SARS-CoV-2.

| | | IC₅₀ | 95% Confidence |
|---|---|---|---|
| Antibody | Series | (μg/ml) | Interval |
| Ab-12 | 1 | 0.09022 | 0.07418-0.1096 |
| | 2 | 0.1035 | 0.07667-0.1387 |
| | 3 | 0.08541 | 0.07014-0.1034 |
| Ab-14 | 1 | 0.1443 | 0.08394-0.2362 |
| | 2 | 0.2298 | 0.1977-0.2673 |
| | 3 | 0.2055 | 0.1467-0.2841 |
| Ab-19 | 1 | NA | NA |
| | 2 | NA | NA |
| | 3 | 0.9627 | 0.5487-4.851 |
| Ab-27 | 1 | 0.04904 | 0.03376-0.07059 |
| | 2 | 0.04447 | 0.03555-0.05574 |
| | 3 | 0.04227 | 0.03366-0.05350 |
| Ab-28 | 1 | NA | NA |
| | 2 | NA | NA |
| | 3 | NA | NA |

### Example 6—Pseudovirus Neutralization

Pseudovirus was made my transfecting LentiX cells with CMV-d8.2, HIV-luc, and pcDNA3.4-SARS2-spike-gp41 tail with lipofectamine 3000. The cells were incubated at 37° C. for 3 days before harvest and filtration (0.45 μm). Pseudovirus is stored at 4° C. or used immediately.

Target cells: 293T-ACE2 transduced cells, seeded 10,000 cells/well in 100 μl day before.

For neutralization assay, 120 μl pseudovirus was incubated with 120 μl ab dilution at RT for 1 hour. Growth media was removed from the plate and replaced with 60 μl pseudovirus/Ab mixture (done in triplicate). The plates were incubated at 37° C. for 48 hours before the cells were lysed with Promega passive lysis buffer followed by luciferase measurement via Promega Bio-Glow.
Plate 2: single dilution at 100 μg/ml scFv-Fc for all 28 antibodies
Plate 4: titration curves of scFv-Fcs from set 1 (Ab 7, Ab 12, Ab2-2, Ab 2-7, Ab2-10)
Plate 6: titration curves of scFv-Fcs from set 2 (Ab 14, Ab 19, Ab 23, Ab 26, Ab 27, Ab 28)
   antibodies from second set were chosen based on competition assay, best binder was chosen for each bin

### Example 7—Engineering Bispecific Antibodies for the SARS-COV-2 Receptor Binding Domain

Why Develop Bispecific Antibodies for SARS-CoV-2. Bispecific (Bs) antibody targeting different epitopes on the same antigen can display enhanced binding affinity (Zhou, 2003). The Bs antibody can also serve as a vaccine alternative or supplement. For example, antibody-dependent enhancement (ADE) is observed in response to SARS-CoV subunit vaccine (Jaume et al., 2012). Also, neutralizing antibodies in individuals who recovered from SARS-CoV-2 infection start to decrease within 2-3 months after infection (Long et al., 2020). Targeting non-overlapping epitopes can mitigate risk of neutralization escape (Baum et al., 2020). A noncompeting pair of neutralizing antibodies exhibited neutralization of SARS-CoV-2 (Wu et al., 2020).

TABLE V
Current Antibody-Based Approaches to SARS-CoV-2.
Reported Neutralizing SARS-CoV-2 Monoclonal Antibodies

| Name | Publication Date |
|---|---|
| CR3022 | Apr. 3, 2020 |
| 47D11 | May 4, 2020 |
| B38 and H4 | May 13, 2020 |
| S309 | May 18, 2020 |
| P2B-2F6 | May 26, 2020 |
| REGN10987 | Jun. 15, 2020 |
| and REGN10933* | |

*Regeneron antibody cocktail “REGN-COV2” first to enter human clinical trials

It has been reported that once an RBD is fixed in the up position by binding to ACE2 on the surface of a target cell, a flexible FPPR can expose the S2′ cleavage site (Cai et al., 2020).

Strategy: Develop a bispecific antibody targeting ACE2 and CR3022 epitopes to fix RBD in the “up” position, thus exposing the S2′ cleavage site and facilitating irreversible S protein transition into more stable postfusion conformation. Thus, will (1) engineer bispecific antibodies that bind to distinct, non-overlapping epitopes on the S protein RBD; (2) demonstrate enhanced binding affinities of bispecific antibodies to RBD epitopes; and (3) determine neutralization potential of bispecific antibodies towards SARS-CoV-2.

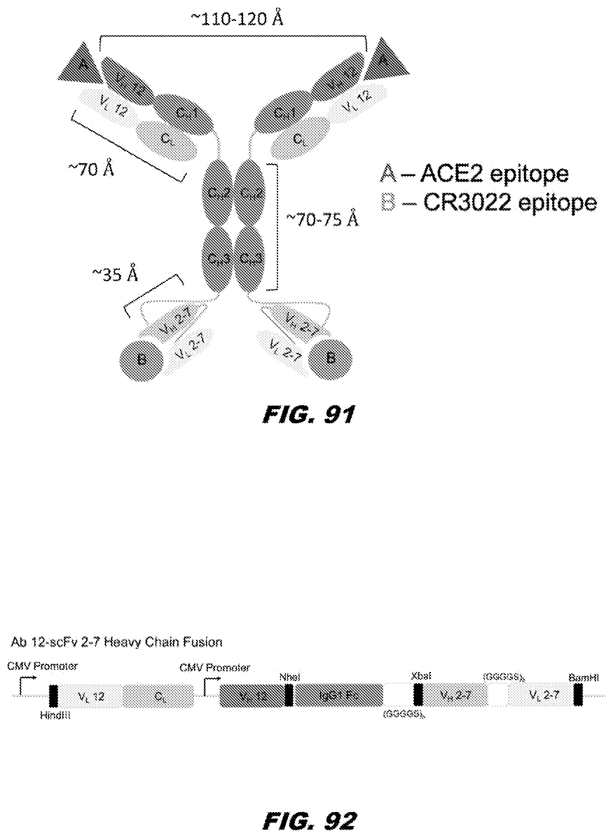

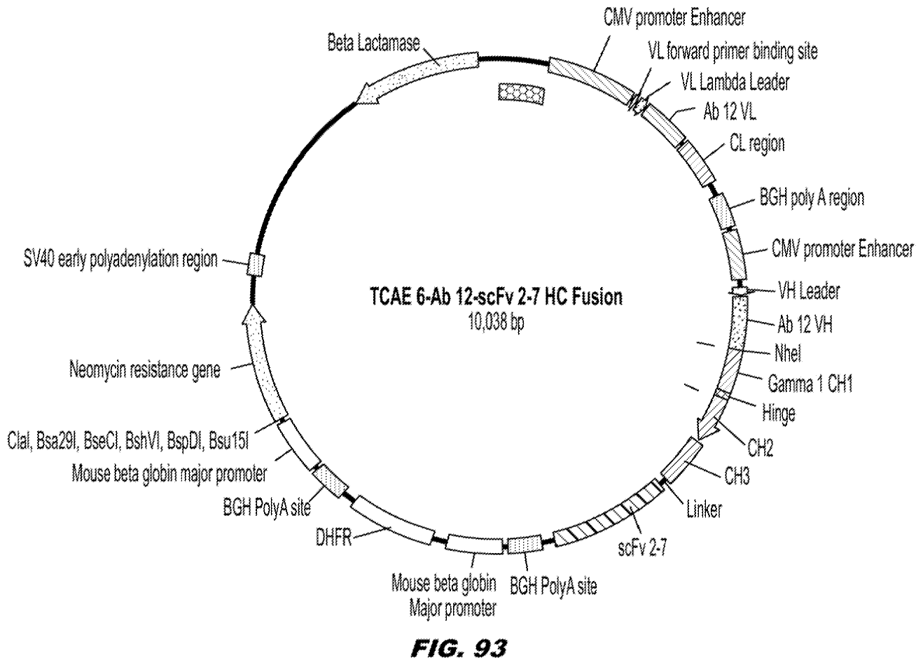

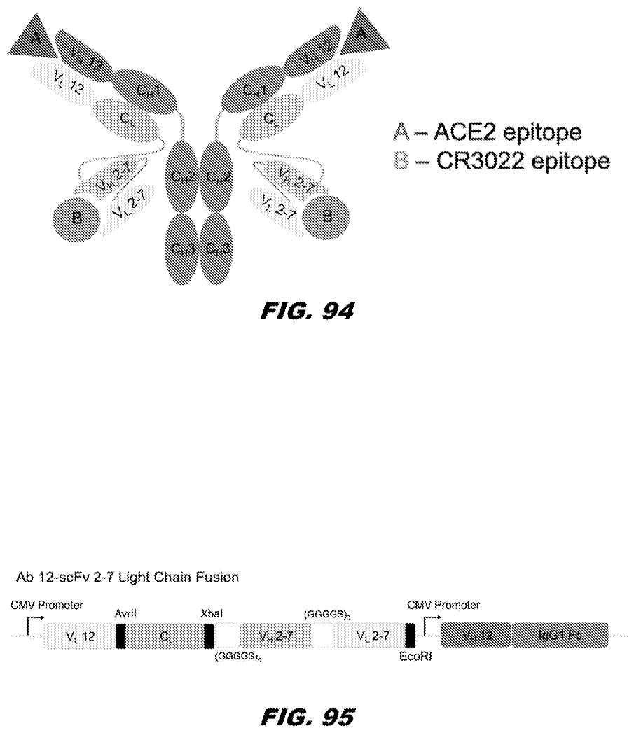

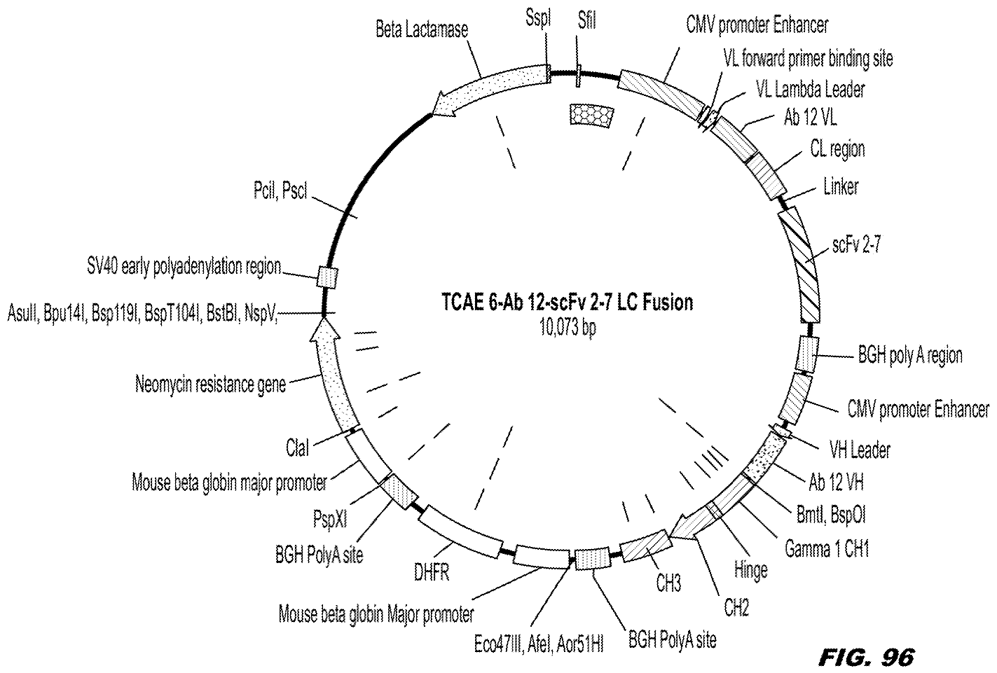

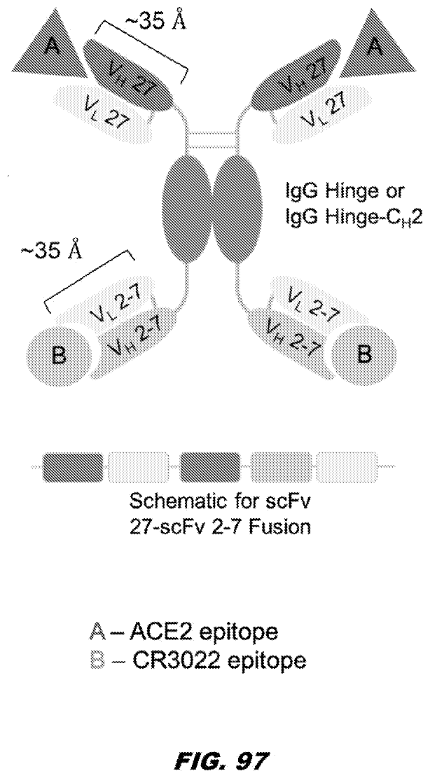

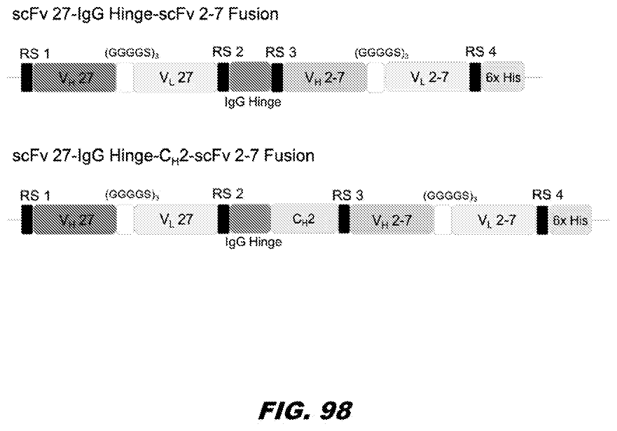

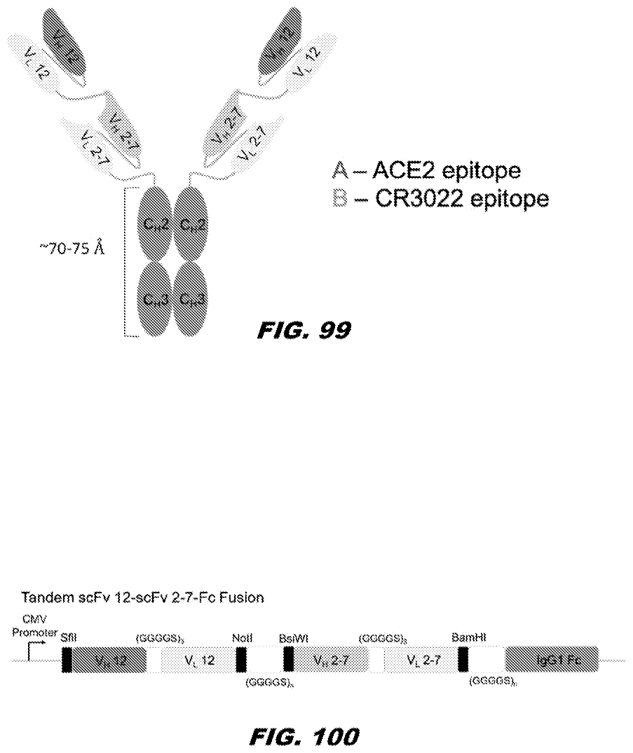

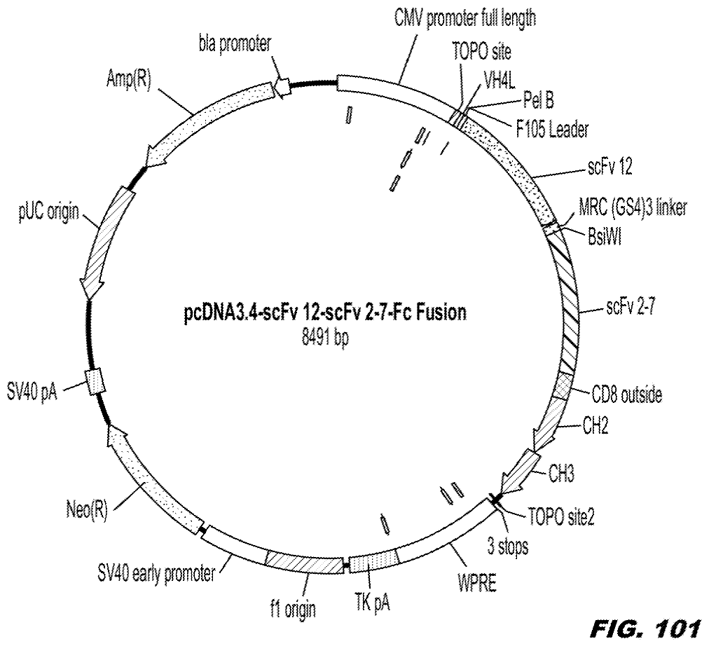

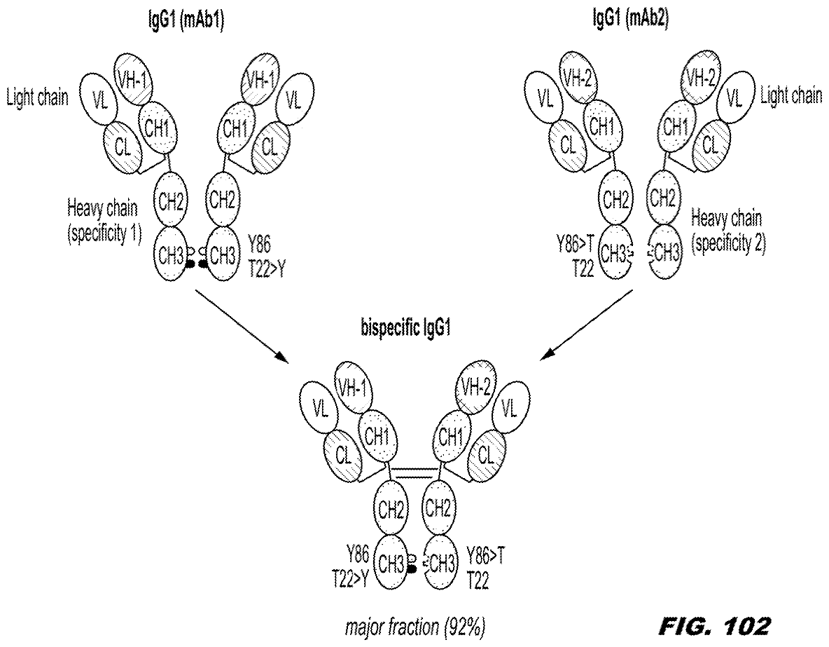

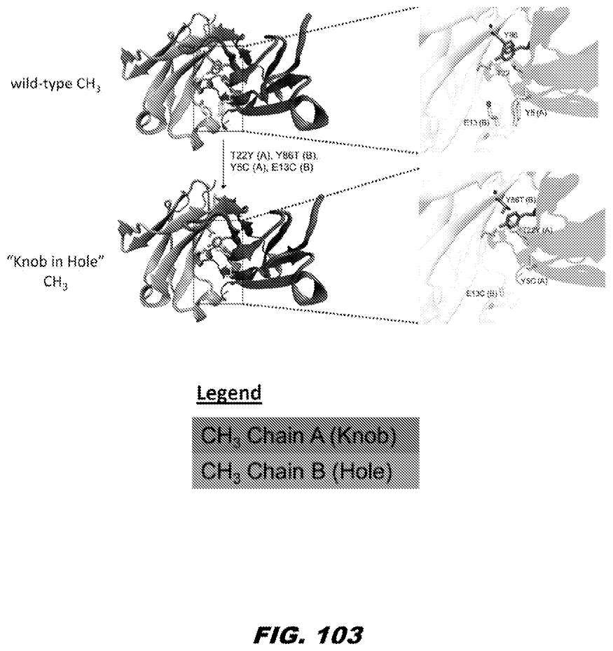

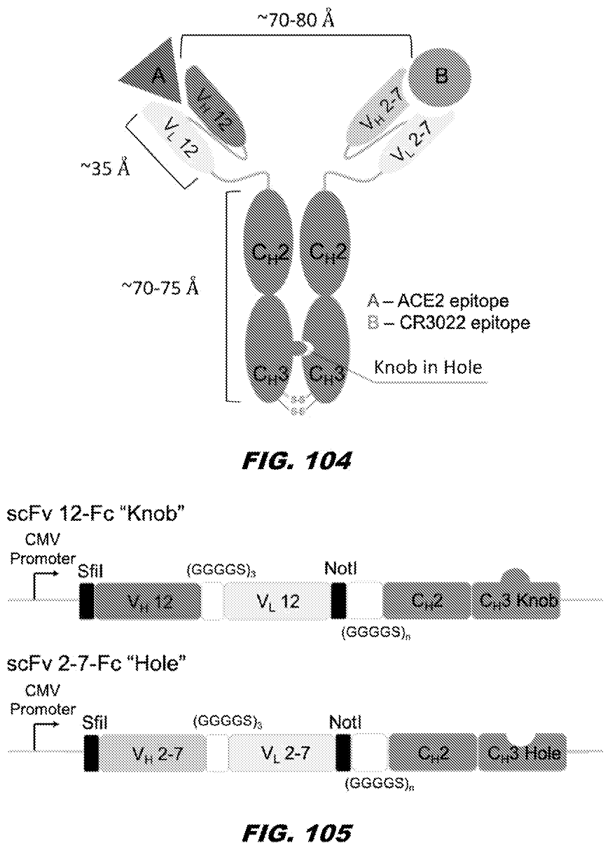

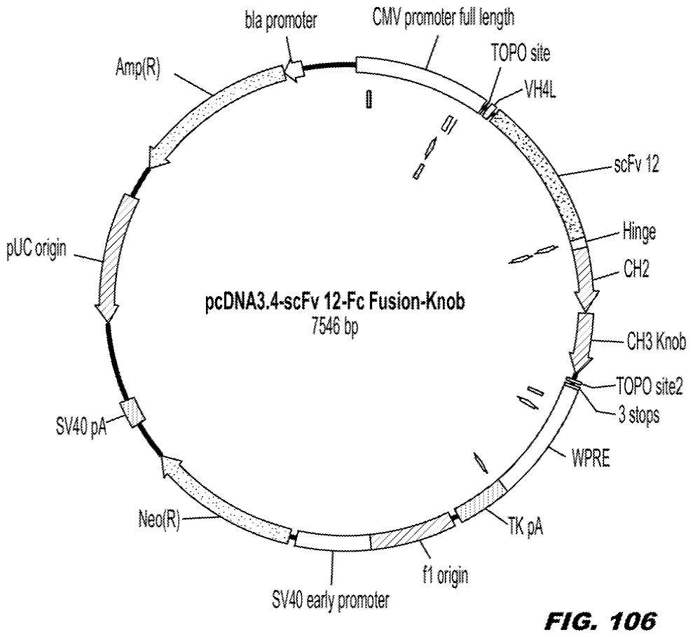

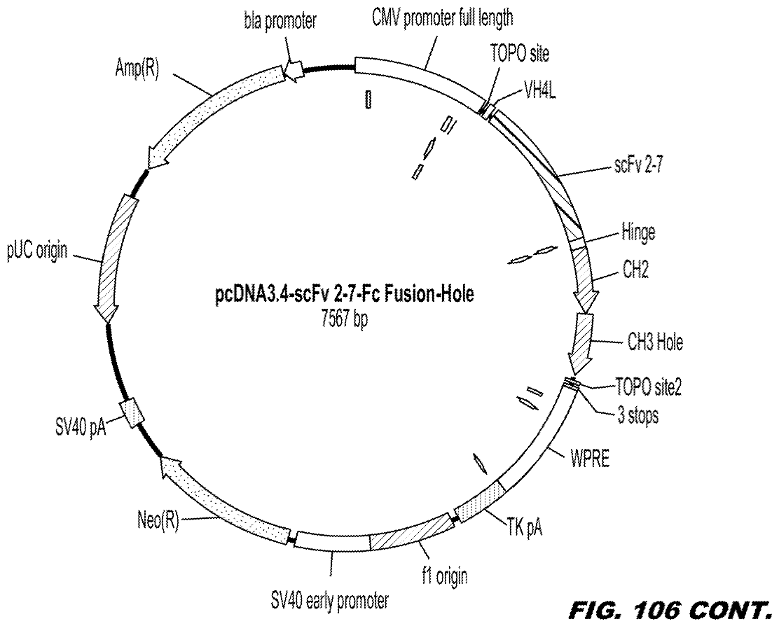

Expression and Purification Strategies. Various examples of constructs useful in generating the Bs antibodies described herein are depicted in the FIGS. 91-109.

### Constructs 1 and 2

Expression: Transfect IgG1-scFv constructs in HEK Expi293F cells
Purification: Protein A Affinity Chromatography

### Construct 3

Expression: Transfect tandem scFv-Fc constructs in HEK Expi293F cells
Purification: Protein A Affinity Chromatography

### Construct 4

Expression: Co-transfect scFv 12-Fc Knob and scFv 2-7-Fc Hole constructs in HEK Expi293F cells
Purification: Protein A Affinity Chromatography

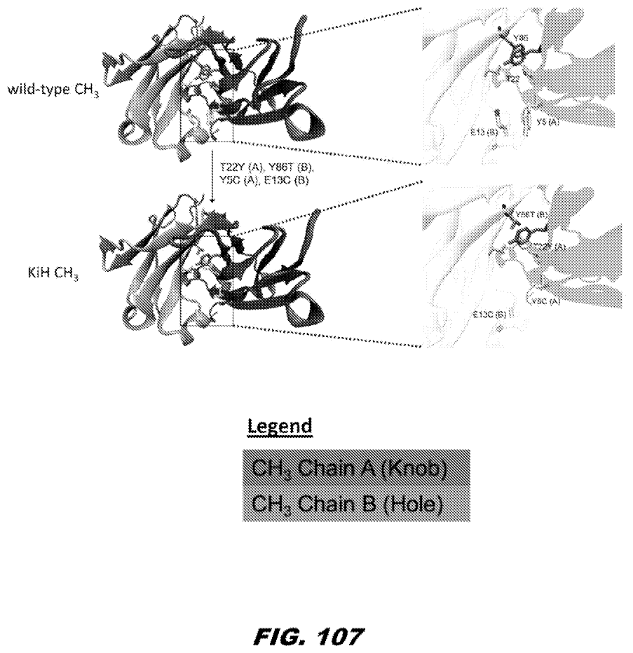

For the KiH Construct 1 Amino Acid Sequence (FIG. 107):

CH₃ Chain A-Mutated AA Sequence-
(SEQ ID NO:_)
GQPREPQVCTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
CH₃ Chain B-Mutated AA Sequence-
(SEQ ID NO:_)
GQPREPQVYVYPPSRDCLTKNQVSLTCLVKGFYPSDIAVEWESNGQPEN
NYKTTPPVLDSDGSFFLTSKLTVDKSRWQQGNVFSCSVMHEALHNHYTQ
KSLSLSPGK

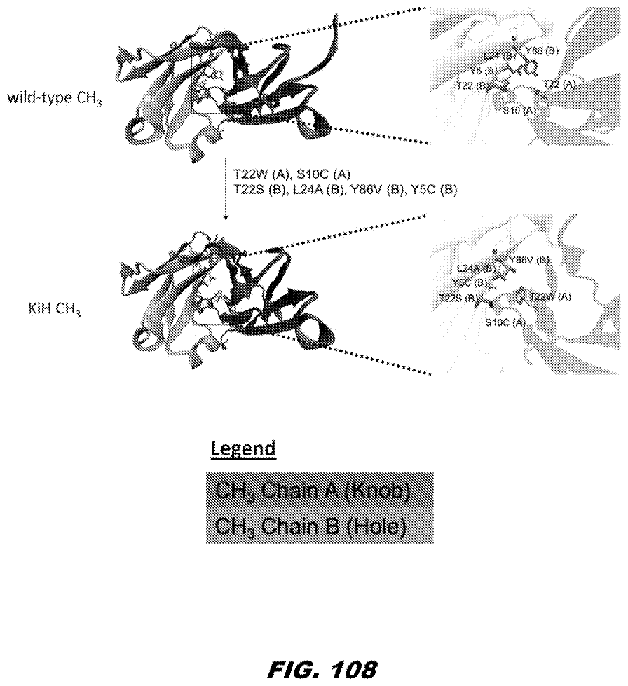

For the KiH Construct 2 (KiH_{S-S}) Amino Acid Sequence (FIG. 108; see Merchant et al., 1998 and Leaver-Fay et al., 2016)

CH₃ Chain A-Mutated AA Sequence-
(SEQ ID NO:_)
GQPREPQVYTLPPCRDELTKNQVSLWCLVKGFYPSDIAVEWESNGQPEN
NYKTTPPVLDSDGSFFLYSKLTVDKSRWQQGNVFSCSVMHEALHNHYTQ
KSLSLSPGK
CH₃ Chain B-Mutated AA Sequence-
(SEQ ID NO:_)
GQPREPQVCTLPPSRDELTKNQVSLSCAVKGFYPSDIAVEWESNGQPEN
NYKTTPPVLDSDGSFFLVSKLTVDKSRWQQGNVFSCSVMHEALHNHYTQ
KSLSLSPGK

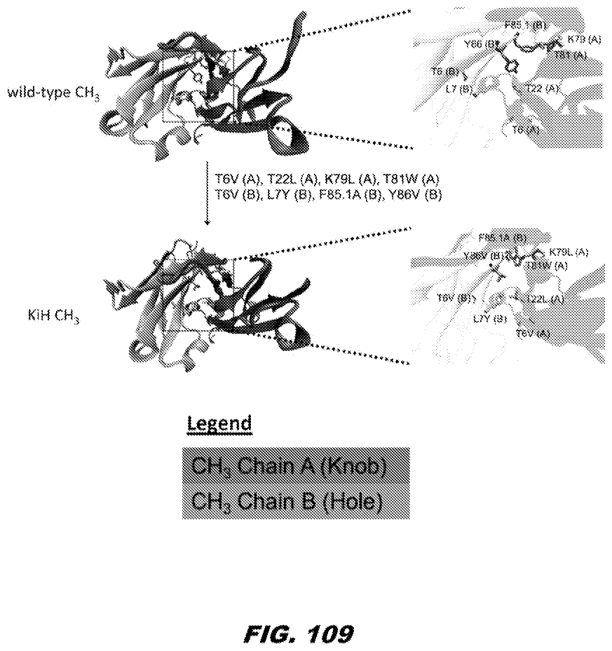

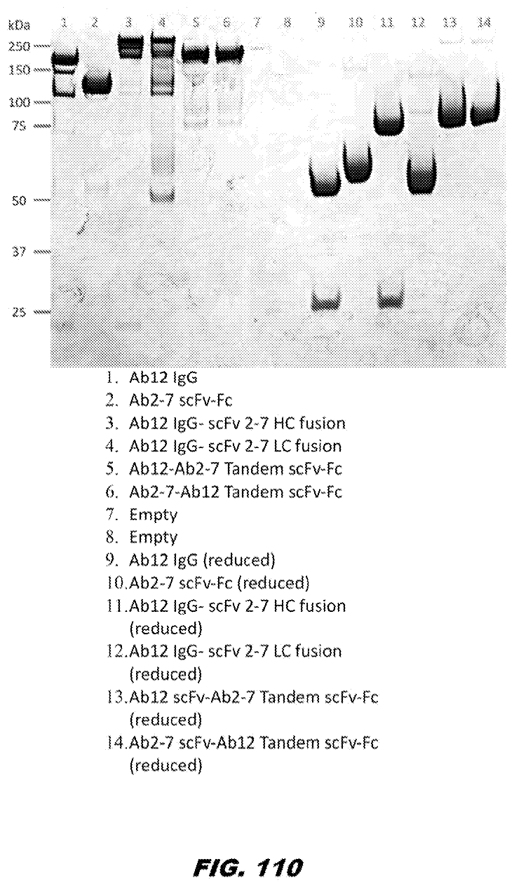

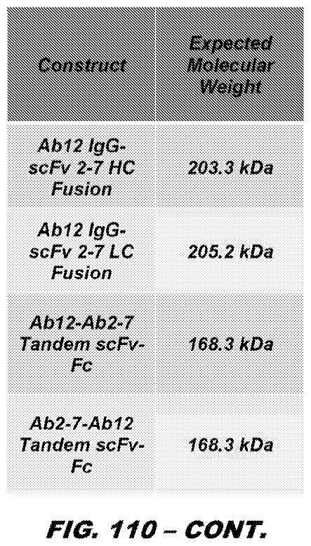

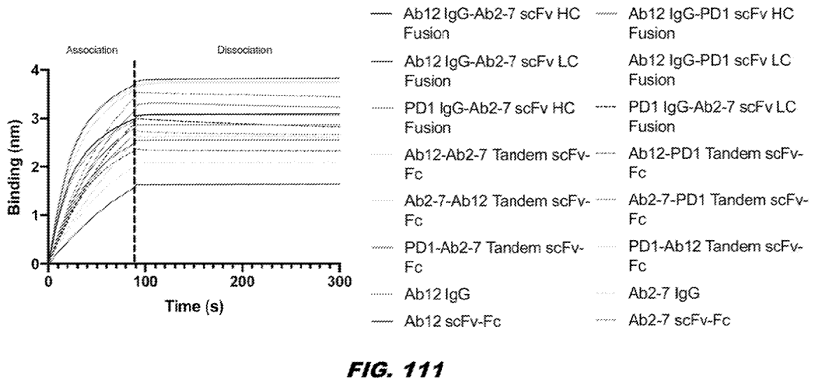

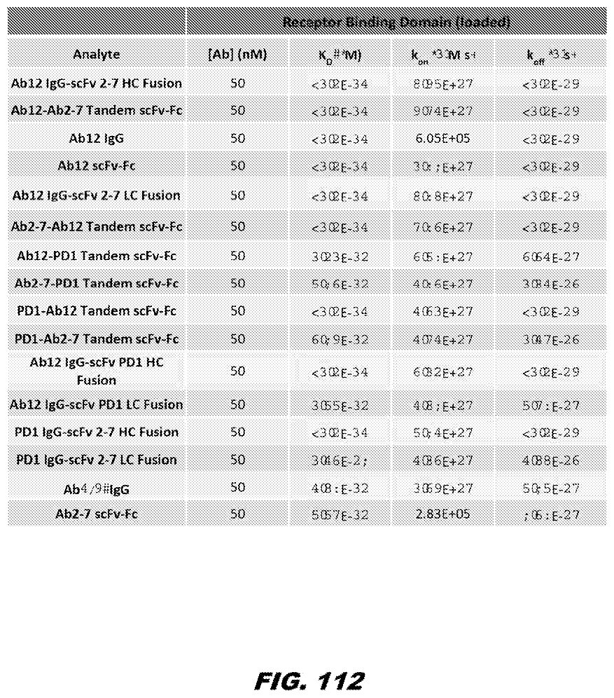

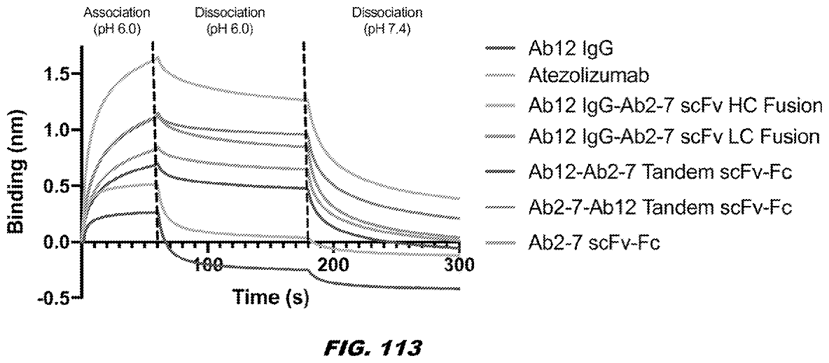

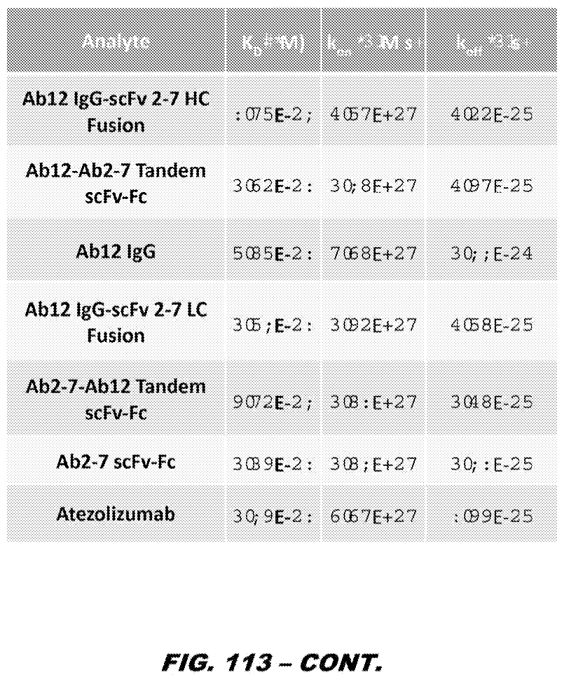

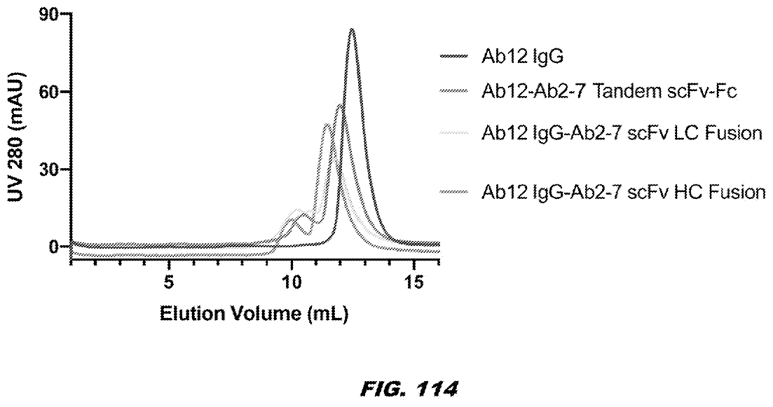

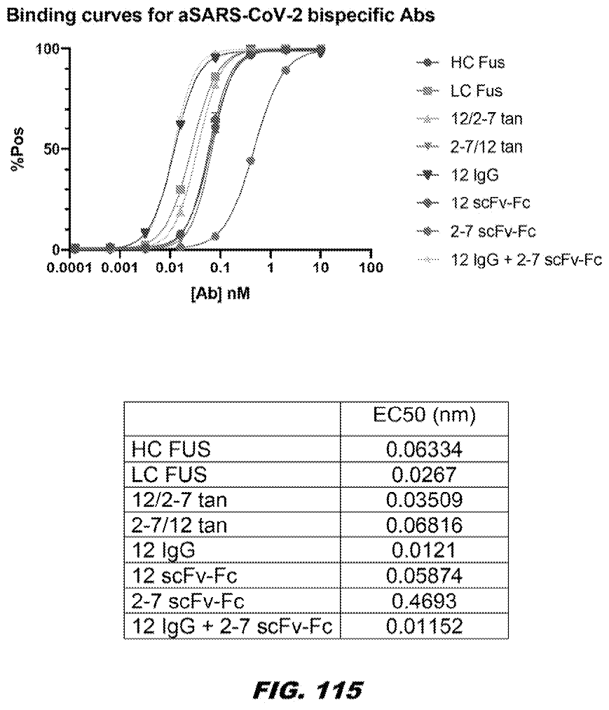

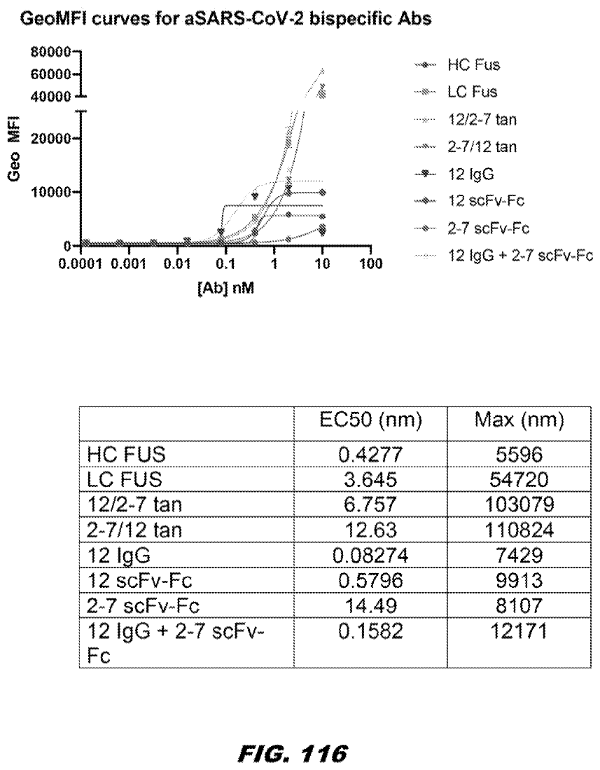

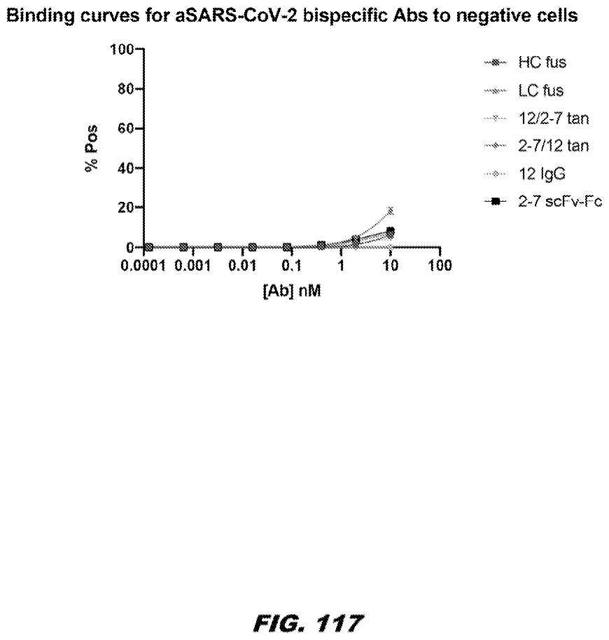

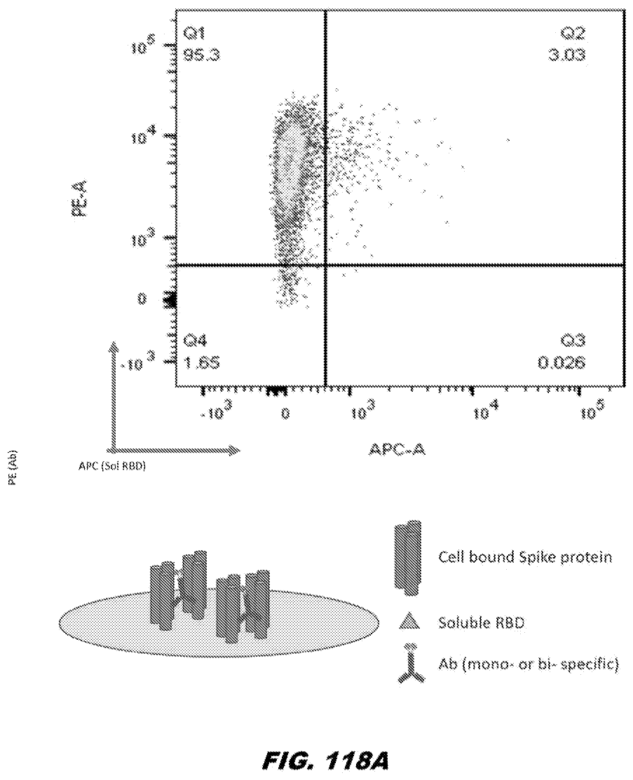

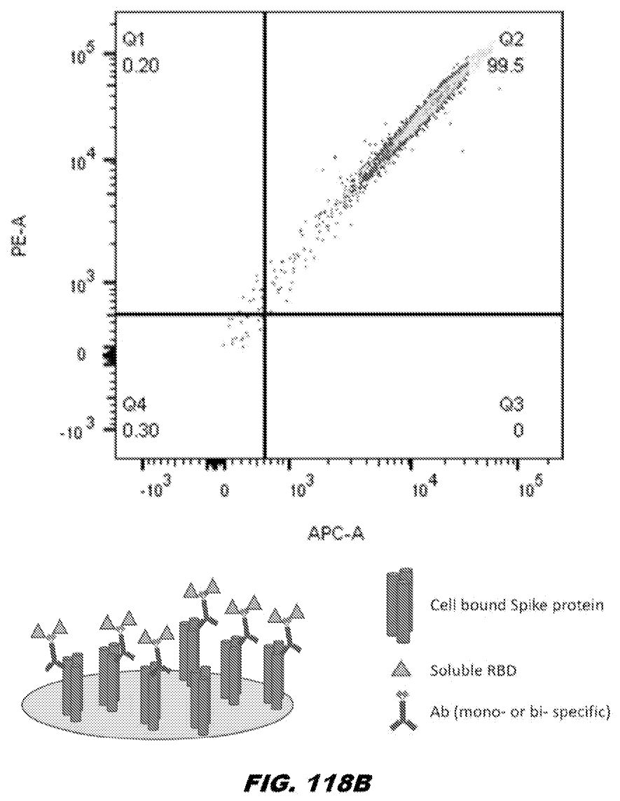

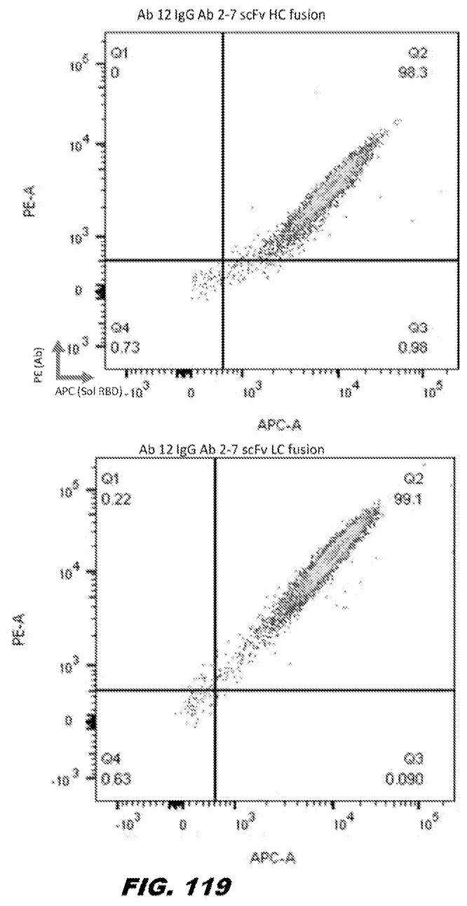

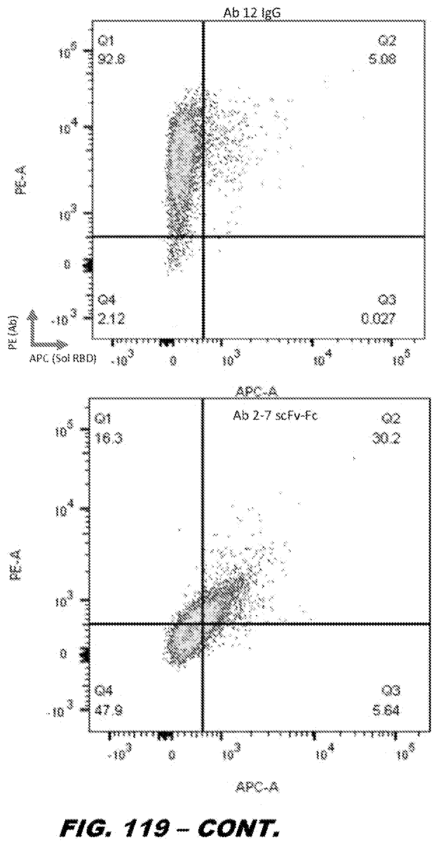

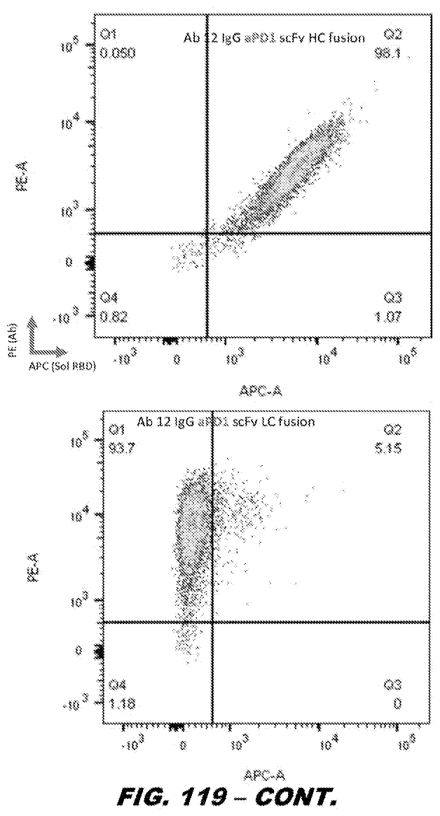

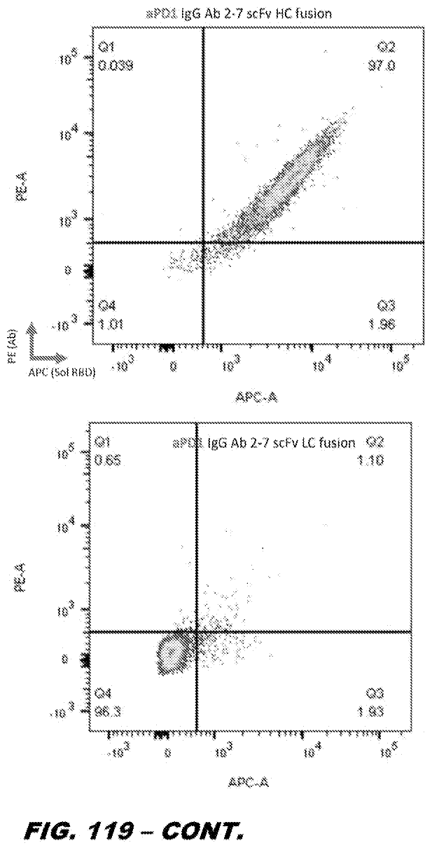

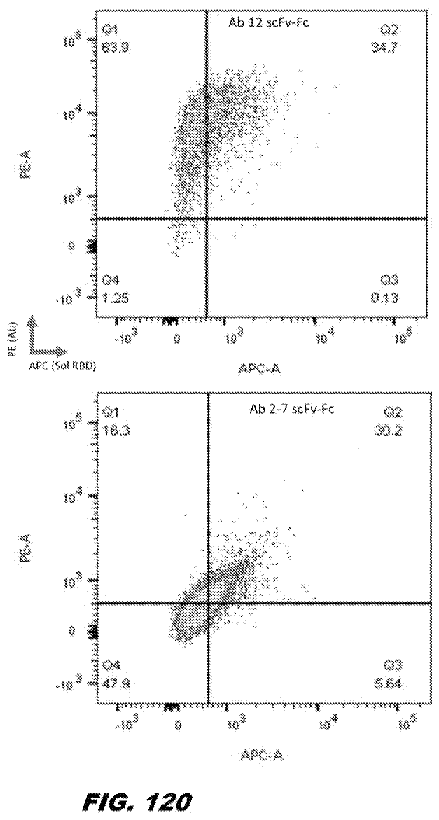

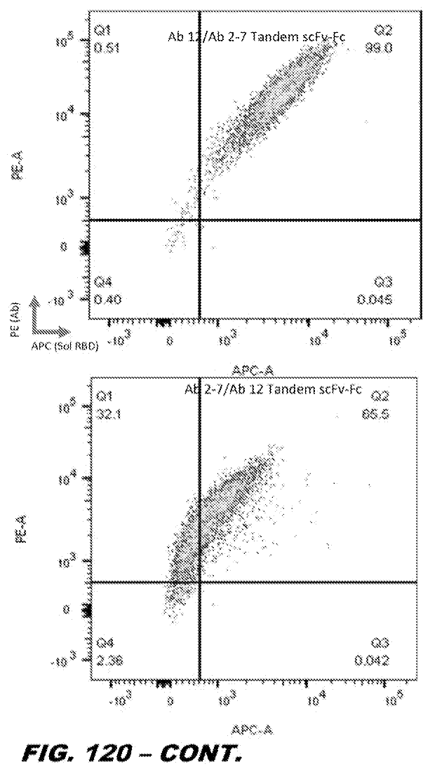

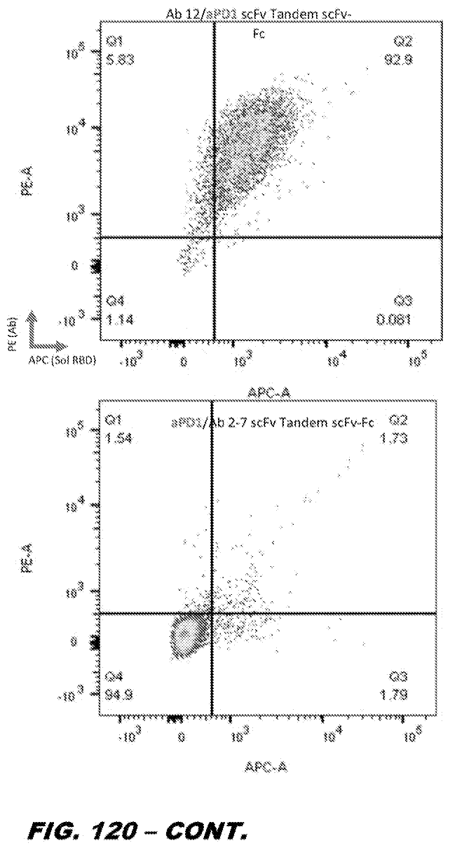

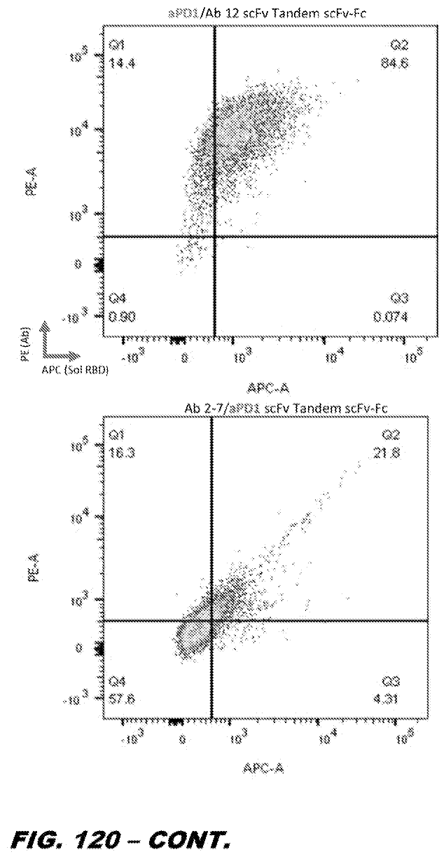

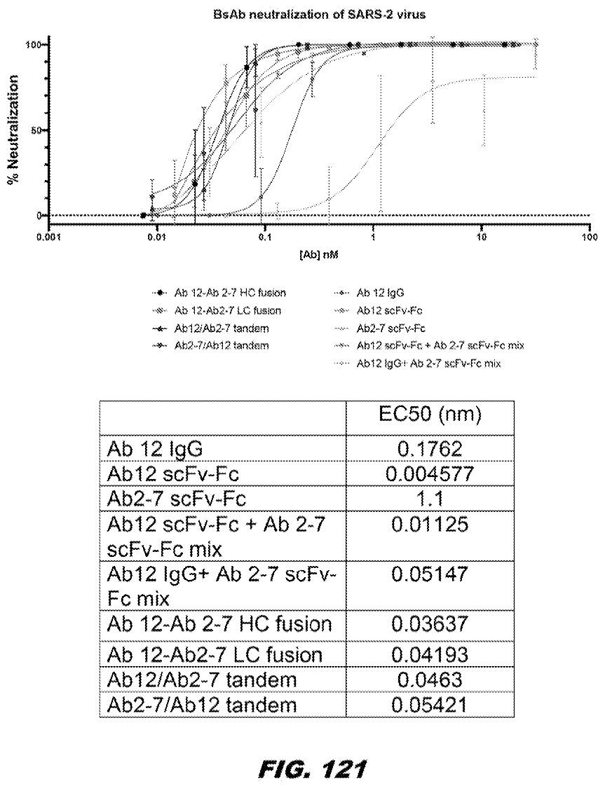

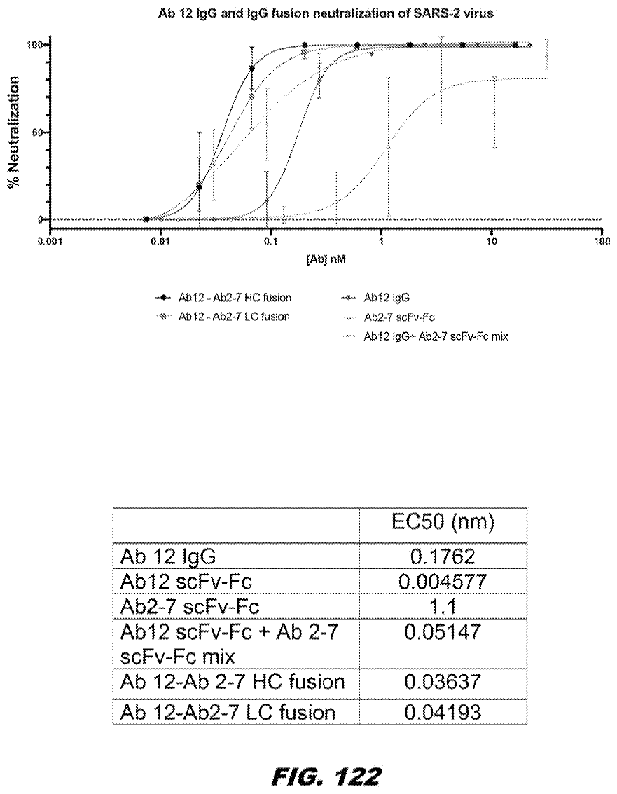

For the KiH Construct 3 (ZW1) Amino Acid Sequence (FIG. 109; see Von Kreudenstein et al., 2013)

CH₃ Chain A-Mutated AA Sequence-
(SEQ ID NO:_)
GQPREPQVYVLPPSRDELTKNQVSLLCLVKGFYPSDIAVEWESNGQPEN
NYLTWPPVLDSDGSFFLYSKLTVDKSRWQQGNVFSCSVMHEALHNHYTQ
KSLSLSPGK
CH₃ Chain B Mutated AA Sequence-
(SEQ ID NO:_)
GQPREPQVYVYPPSRDELTKNQVSLTCLVKGFYPSDIAVEWESNGQPEN
NYKTTPPVLDSDGSFALVSKLTVDKSRWQQGNVFSCSVMHEALHNHYTQ
KSLSLSPGK

Experiments to be conducted.

### (a) Competition ELISA

96-well plates will be coated with RBD monomer. The ACE2 epitope will be blocked with ACE2 polypeptides. Wells will then be incubated with bispecific Abs 1-3 (primary Ab) then subsequently will be incubated with a secondary Ab. Absorbance will then be measured.

96-well plates will be coated with RBD monomer. The CR3022 epitope will be blocked with CR3022 Fab. Wells will then be incubated with bispecific Abs 1-3 (primary Ab) then subsequently will be incubated with secondary Ab. Absorbance will then be measured.

96-well plates will be coated with RBD monomer. The ACE2 and CR3022 epitopes will be blocked with ACE2 polypeptides and CR3022 Fab, respectively. Wells will then be incubated with bispecific Abs 1-3 (primary Ab) then subsequently will be incubated with secondary Ab. Absorbance will then be measured.

96-well plates will be coated with RBD monomer then incubated with bispecific Abs 1-3 (primary Ab). Wells will then be incubated with secondary Ab. Absorbance will then be measured. Positive control—α-IgG Fc or α-His primary Ab; Negative controls—BSA and/or nonbinding primary Ab.
(b) Bio-layer interferometry (Octet® System)
(c) Fluorescence-activated cell sorting (FACS)
(d) Pseudo-virus neutralization assays

### Example 8—Thermal Shift Assay

Thermal shift assays (or differential scanning calorimetry/DSC) is used to measure the unfolding of a protein in real time via hydrophobic interactions. Protein is incubated in the presence of a fluorescent dye (SYPRO Orange) and in the folded state, there is low binding of the dye to the protein. As the temperature of the sample increases, the protein will unfold, gradually exposing the hydrophobic core of the protein to the SYPRO Orange in solution increasing the fluorescent signal. SYPRO orange fluorescence is quenched by H2O, hence there is lower fluorescence with a folded protein. (See Huynh, Kathy, and Carrie L Partch. “Analysis of protein stability and ligand interactions by thermal shift assay.” Current protocols in protein science vol. 79 28.9.1-28.9.14. 2 Feb. 2015, doi:10.1002/0471140864.ps2809s79; King, Amy C et al. “High-throughput measurement, correlation analysis, and machine-learning predictions for pH and thermal stabilities of Pfizer-generated antibodies.” Protein science: a publication of the Protein Society vol. 20, 9 (2011): 1546-57. doi:10.1002/pro.680).

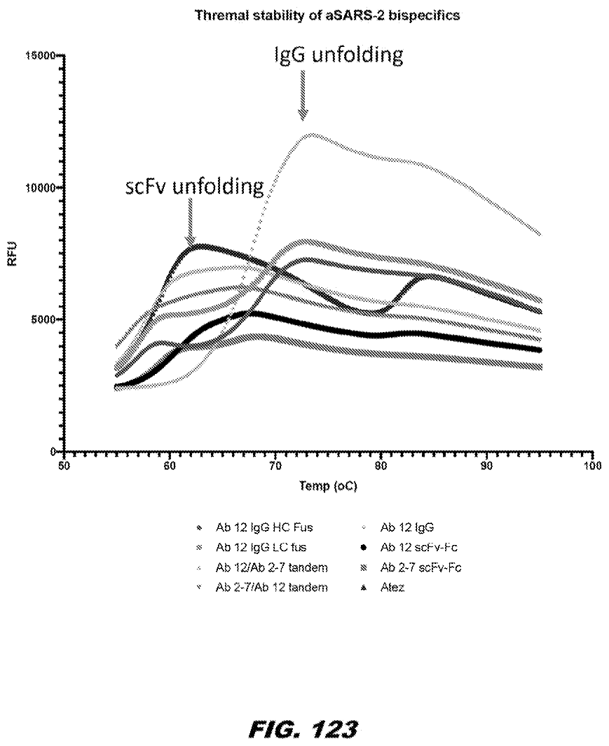

A snap frozen, clinical prep of atezolizumab was used as an IgG control in this experiment. As shown in FIG. 123, the scFv-Fcs begin to unfold at lower temperatures compared to the IgG based molecules. For the IgG fusion antibodies, two distinct peaks are observed, one around 58° C. which is close to that of the scFv-Fcs, and one around 72° C., which matches the Ab 12 IgG construct (FIG. 123), indicating that in the fusion proteins, the scFv portion denatures first, while the IgG remains intact.

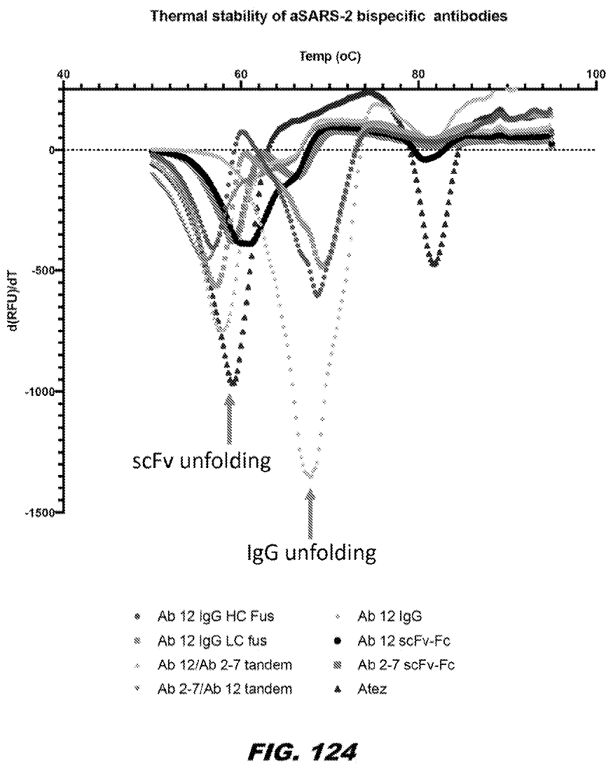

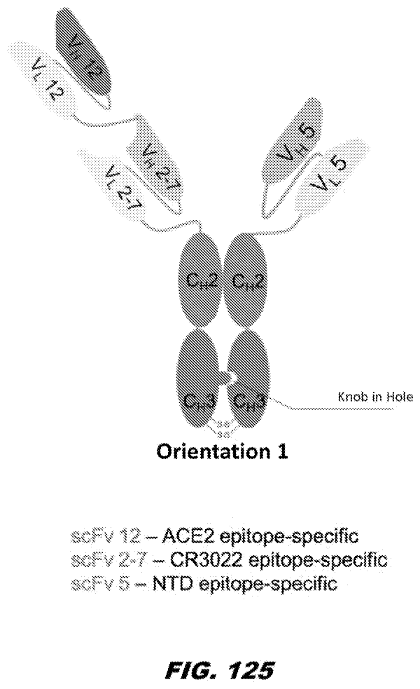

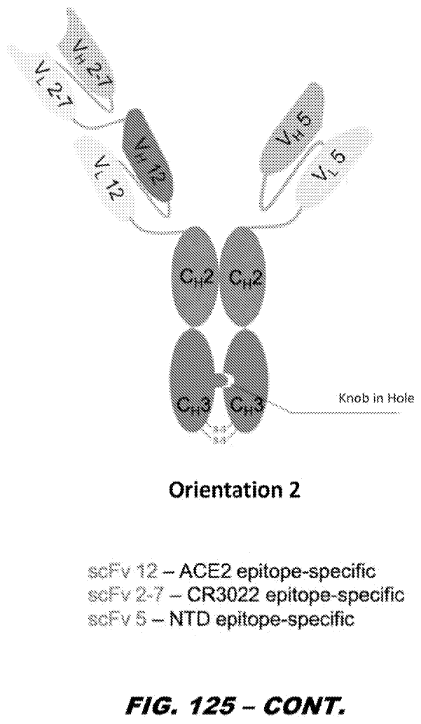

A snap frozen, clinical prep of atezolizumab was used as an IgG control in this experiment. As shown in FIG. 124, the scFv-Fcs begin to unfold at lower temperatures compared to the IgG based molecules. For the IgG fusion antibodies, two distinct peaks are observed, one around 58° C. which is close to that of the scFv-Fcs, and one around 72° C., which matches the Ab 12 IgG construct (FIG. 124), indicating that in the fusion proteins, the scFv portion denatures first, while the IgG remains intact.

TABLE IV
Overall Kinetic Data

| | [Ab] | K_{D} | kₒₙ | k_{off} |
|---|---|---|---|---|
| Analyte | (nM) | (M) | (1/M s) | (1/s) |
| | Human FcRn | | | |
| Ab12 IgG-scFv 2-7 HC | 500 | 8.53E−09 | 2.35E+05 | 2.00E−03 |
| Fusion | | | | |
| Ab12-Ab2-7 Tandem | 500 | 1.40E−08 | 1.96E+05 | 2.75E−03 |
| scFv-Fc | | | | |
| Ab12 IgG | 500 | 3.63E−08 | 5.46E+05 | 1.99E−02 |
| Ab12 scFv-Fc | — | — | — | — |
| Ab12 IgG-scFv 2-7 LC | 500 | 1.39E−08 | 1.70E+05 | 2.36E−03 |
| Fusion | | | | |
| Ab2-7-Ab12 Tandem | 500 | 7.50E−09 | 1.68E+05 | 1.26E−03 |
| scFv-Fc | | | | |
| Ab12-PD1 Tandem scFv- | — | — | — | — |
| Fc | | | | |
| Ab2-7-PD1 Tandem | — | — | — | — |
| scFv-Fc | | | | |
| PD1-Ab12 Tandem scFv- | — | — | — | — |
| Fc | | | | |
| PD1-Ab2-7 Tandem | — | — | — | — |
| scFv-Fc | | | | |
| Ab12 IgG-scFv PD1 HC | — | — | — | — |
| Fusion | | | | |
| Ab12 IgG-scFv PD1 LC | — | — | — | — |
| Fusion | | | | |
| PD1 IgG-scFv 2-7 HC | — | — | — | — |
| Fusion | | | | |
| PD1 IgG-scFv 2-7 LC | — | — | — | — |
| Fusion | | | | |
| Ab2-7 IgG | — | — | — | — |
| Ab2-7 scFv-Fc | 500 | 1.17E−08 | 1.69E+05 | 1.98E−03 |
| Atezolizumab | 500 | 1.97E−08 | 4.45E+05 | 8.77E−03 |
| | Receptor Binding Domain (loaded) | | | |
| Ab12 IgG-scFv 2-7 HC | 50 | <1.0E−12 | 6.73E+05 | <1.0E−07 |
| Fusion | | | | |
| Ab12-Ab2-7 Tandem | 50 | <1.0E−12 | 7.52E+05 | <1.0E−07 |
| scFv-Fc | | | | |
| Ab12 IgG | 50 | <1.0E−12 | 6.05E+05 | <1.0E−07 |
| Ab12 scFv-Fc | 50 | <1.0E−12 | 1.89E+05 | <1.0E−07 |
| Ab12 IgG-scFv 2-7 LC | 50 | <1.0E−12 | 6.86E+05 | <1.0E−07 |
| Fusion | | | | |
| Ab2-7-Ab12 Tandem | 50 | <1.0E−12 | 5.84E+05 | <1.0E−07 |
| scFv-Fc | | | | |
| Ab12-PD1 Tandem scFv- | 50 | 1.01E−10 | 4.38E+05 | 4.42E−05 |
| Fc | | | | |
| Ab2-7-PD1 Tandem | 50 | 3.94E−10 | 2.84E+05 | 1.12E−04 |
| scFv-Fc | | | | |
| PD1-Ab12 Tandem scFv- | 50 | <1.0E−12 | 2.41E+05 | <1.0E−07 |
| Fc | | | | |
| PD1-Ab2-7 Tandem | 50 | 4.97E−10 | 2.52E+05 | 1.25E−04 |
| scFv-Fc | | | | |
| Ab12 IgG-scFv PD1 HC | 50 | <1.0E−12 | 4.10E+05 | <1.0E−07 |
| Fusion | | | | |
| Ab12 IgG-scFv PD1 LC | 50 | 1.33E−10 | 2.69E+05 | 3.58E−05 |
| Fusion | | | | |
| PD1 IgG-scFv 2-7 HC | 50 | <1.0E−12 | 3.92E+05 | <1.0E−07 |
| Fusion | | | | |
| PD1 IgG-scFv 2-7 LC | 50 | 1.24E−09 | 2.14E+05 | 2.66E−04 |
| Fusion | | | | |
| Ab2-7 IgG | 50 | 2.68E−10 | 1.47E+05 | 3.93E−05 |
| Ab2-7 scFv-Fc | 50 | 3.35E−10 | 2.83E+05 | 9.48E−05 |
| Atezolizumab | — | — | — | — |
| | Spike Trimer (loaded Ab at 50 nM) | | | |
| Ab12 IgG-scFv 2-7 HC | 11.3 | <1.0E−12 | 3.14E+07 | <1.0E−07 |
| Fusion | | | | |
| Ab12-Ab2-7 Tandem | 11.3 | <1.0E−12 | 5.46E+07 | <1.0E−07 |
| scFv-Fc | | | | |
| Ab12 IgG | 11.3 | <1.0E−12 | 9.24E+07 | <1.0E−07 |
| Ab12 scFv-Fc | 11.3 | <1.0E−12 | 7.34E+07 | <1.0E−07 |
| Ab12 IgG-scFv 2-7 LC | 11.3 | <1.0E−12 | 6.92E+07 | <1.0E−07 |
| Fusion | | | | |
| Ab2-7-Ab12 Tandem | 11.3 | <1.0E−12 | 8.29E+07 | <1.0E−07 |
| scFv-Fc | | | | |
| Ab12-PD1 Tandem | 11.3 | <1.0E−12 | 3.41E+07 | <1.0E−07 |
| scFv-Fc | | | | |
| Ab2-7-PD1 Tandem | 11.3 | <1.0E−12 | 3.57E+07 | <1.0E−07 |
| scFv-Fc | | | | |
| PD1-Ab12 Tandem | 11.3 | <1.0E−12 | 3.72E+07 | <1.0E−07 |
| scFv-Fc | | | | |
| PD1-Ab2-7 Tandem | 11.3 | <1.0E−12 | 3.01E+07 | <1.0E−07 |
| scFv-Fc | | | | |
| Ab12 IgG-scFv PD1 HC | 11.3 | <1.0E−12 | 3.53E+07 | <1.0E−07 |
| Fusion | | | | |
| Ab12 IgG-scFv PD1 LC | 11.3 | <1.0E−12 | 2.52E+07 | <1.0E−07 |
| Fusion | | | | |
| PD1 IgG-scFv 2-7 HC | 11.3 | <1.0E−12 | 5.99E+07 | <1.0E−07 |
| Fusion | | | | |
| PD1 IgG-scFv 2-7 LC | 11.3 | <1.0E−12 | 1.03E+08 | <1.0E−07 |
| Fusion | | | | |
| Ab2-7 IgG | 11.3 | <1.0E−12 | 4.31E+07 | <1.0E−07 |
| Ab2-7 scFv-Fc | 11.3 | <1.0E−12 | 6.68E+07 | <1.0E−07 |
| Atezolizumab | — | — | — | — |

### Example 9—Engineering Trispecific Antibodies for the SARS-COV-2 Receptor Binding Domain

Constructs in this example herein use the tandem scFv (Ab12/Ab2-7) in either order on the Fc domain with the knob and the mono scFv (Ab5) on the hole side. This can also be done the other direction or with other arrangements linked scFv constructs to create higher order multispecific antibodies.

The tandem knob construct was expressed with a c-terminal 6× His (HHHHHH) and the hole construct with a c-terminal FLAG (DYKDDDDK) or a C9 (TETSQVAPA) tag. These tags were added to allow for two step purification to isolate only heterodimers (first purify via Ni-NTA, then purify the eluted protein with anti-FLAG or -C9 conjugated resins). The heterodimer can have both the His tag from the knob and the FLAG or C9 tag from the hole. We started with C9 tags on the hole but due to difficulty purifying, we switched to FLAG which has commercial resins. Recent purifications show that after Ni-NTA purification, the resultant bands are mostly in the heterodimer state. However, these tags are arbitrary and can be switched out for any known purification tag that does not interfere with antibody stability.

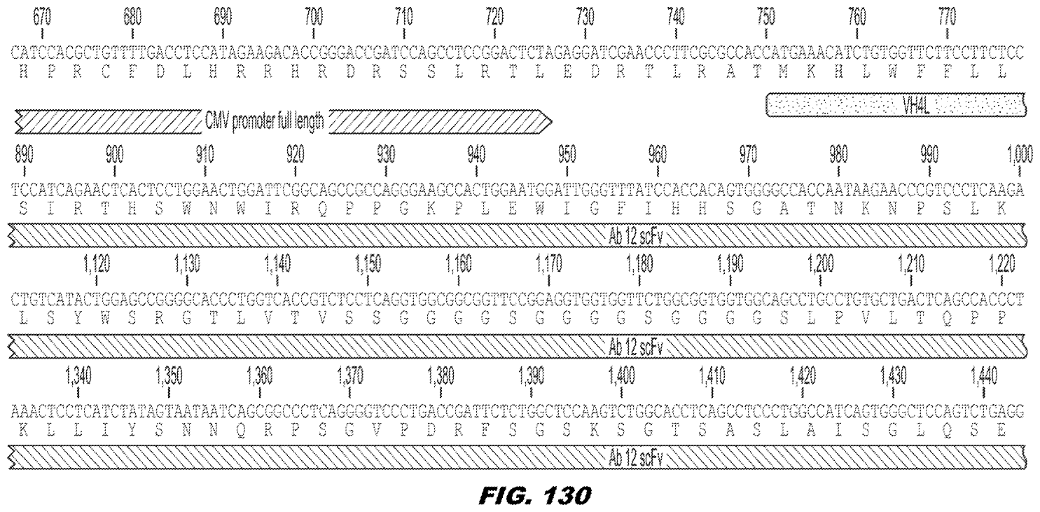

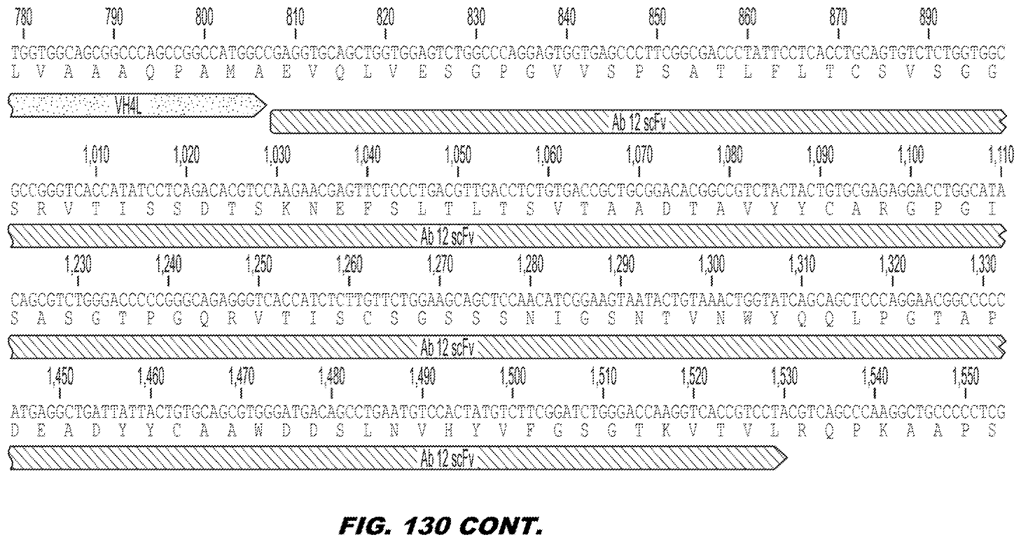

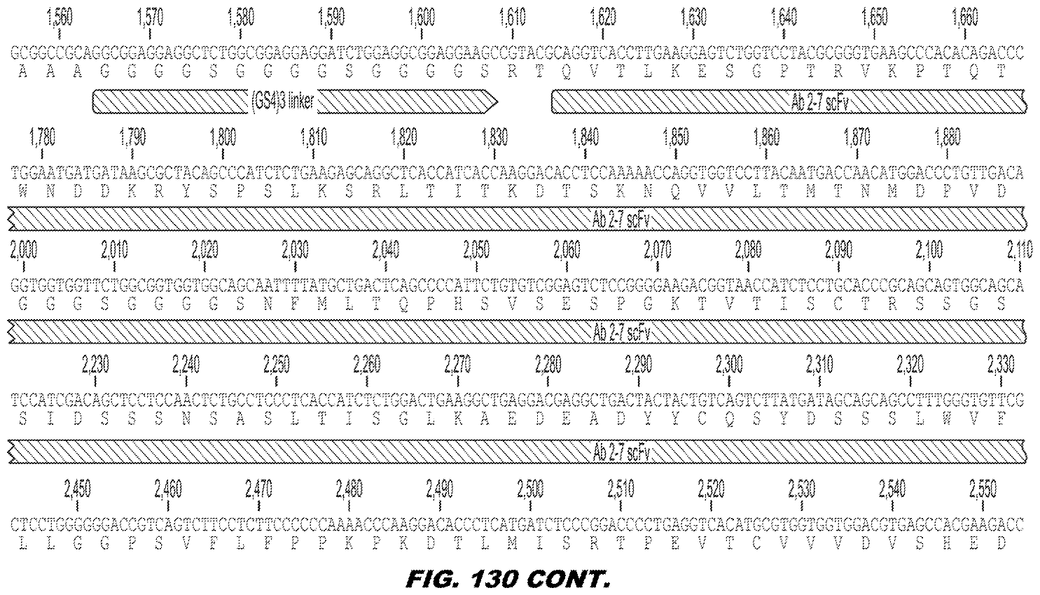

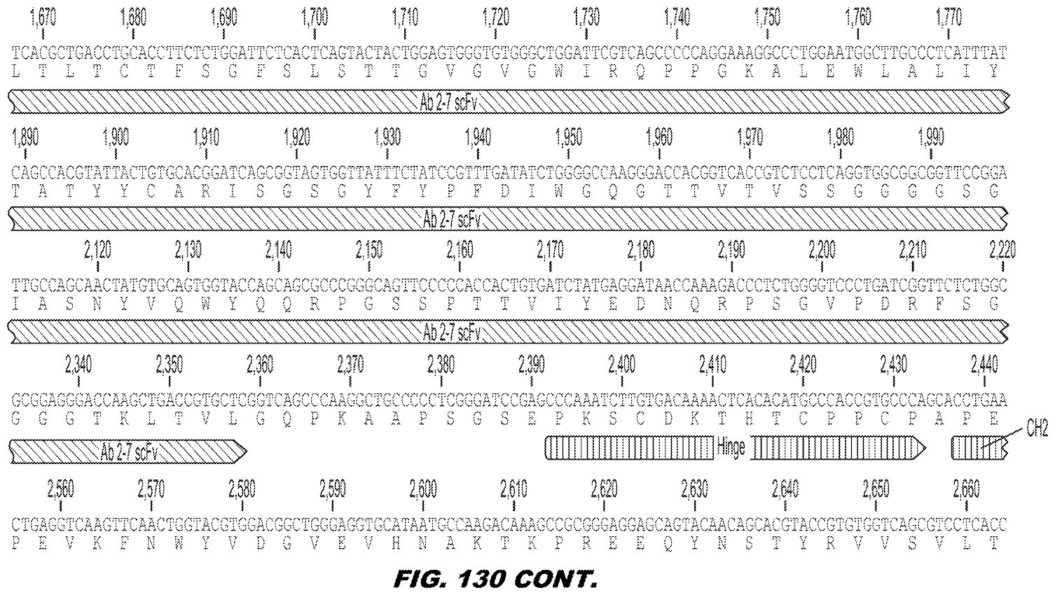

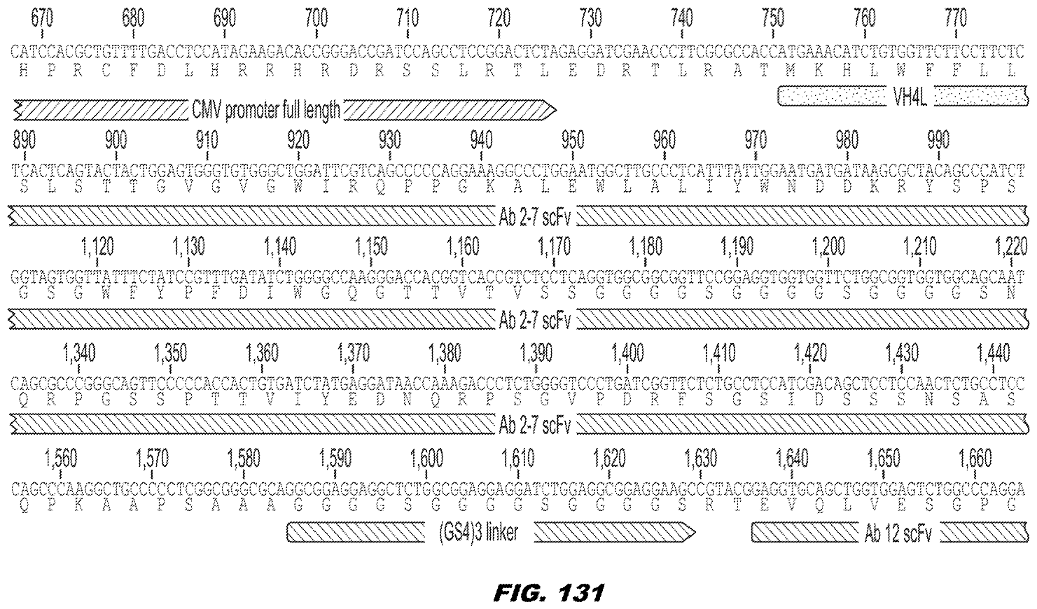

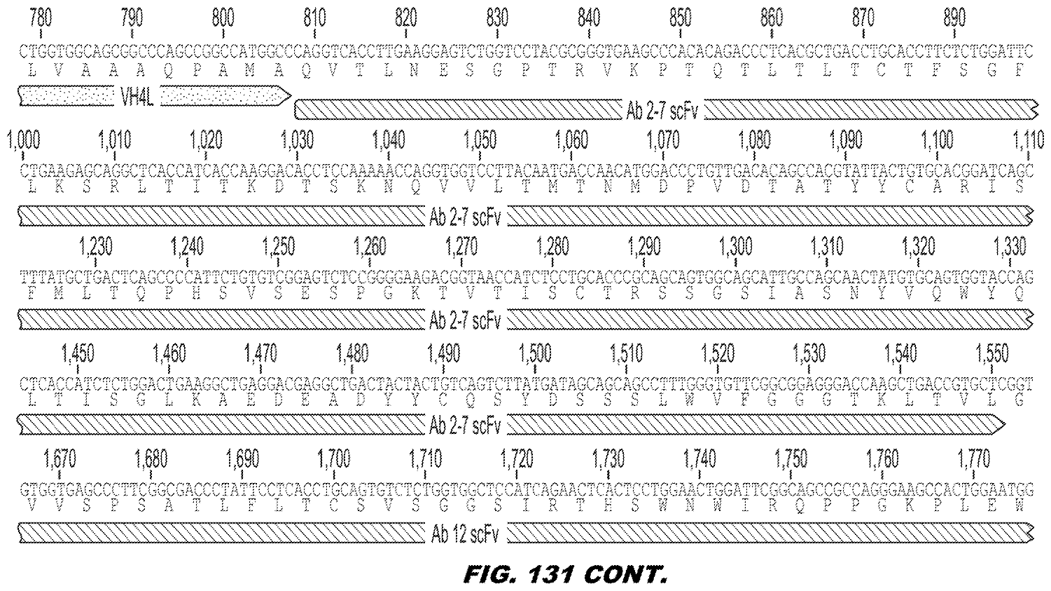

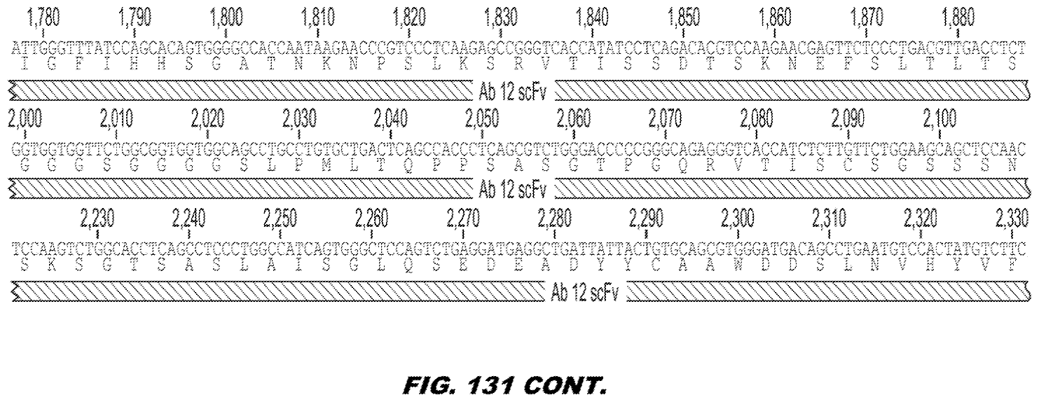

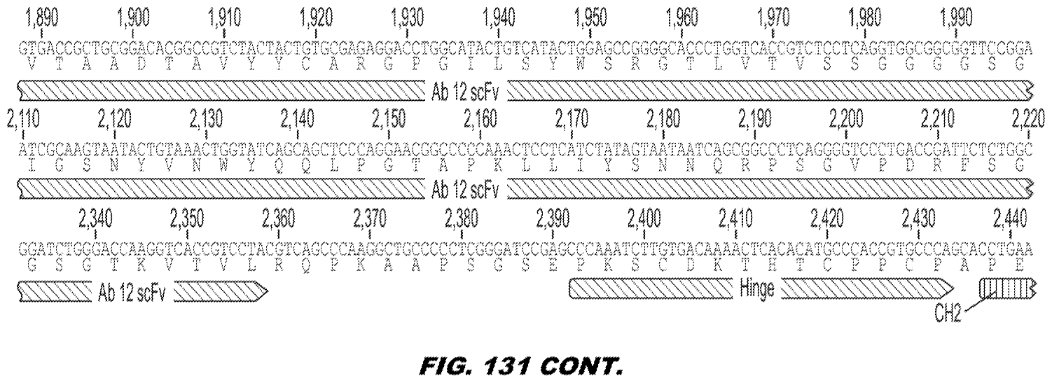

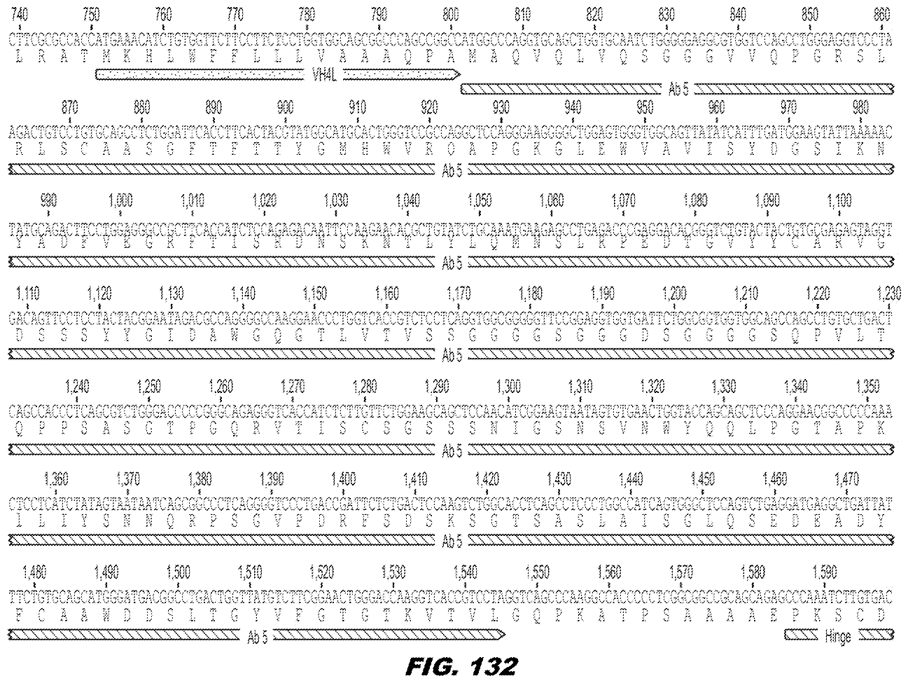

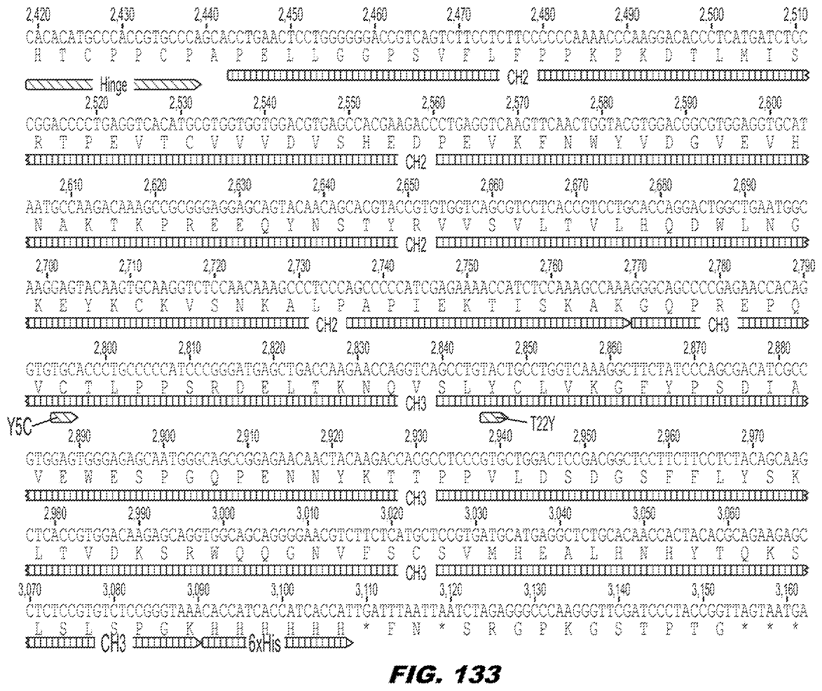

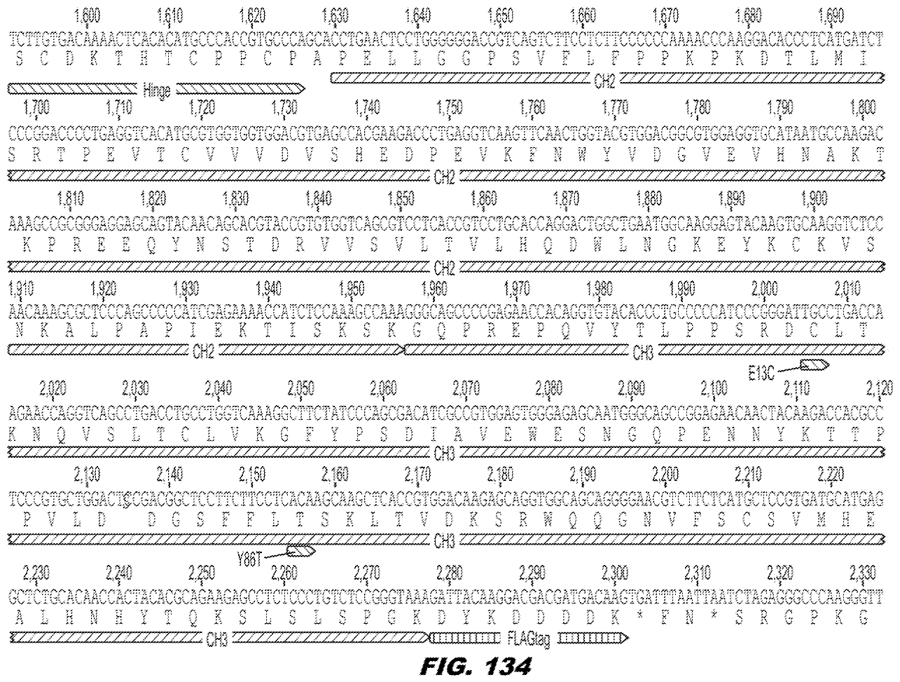

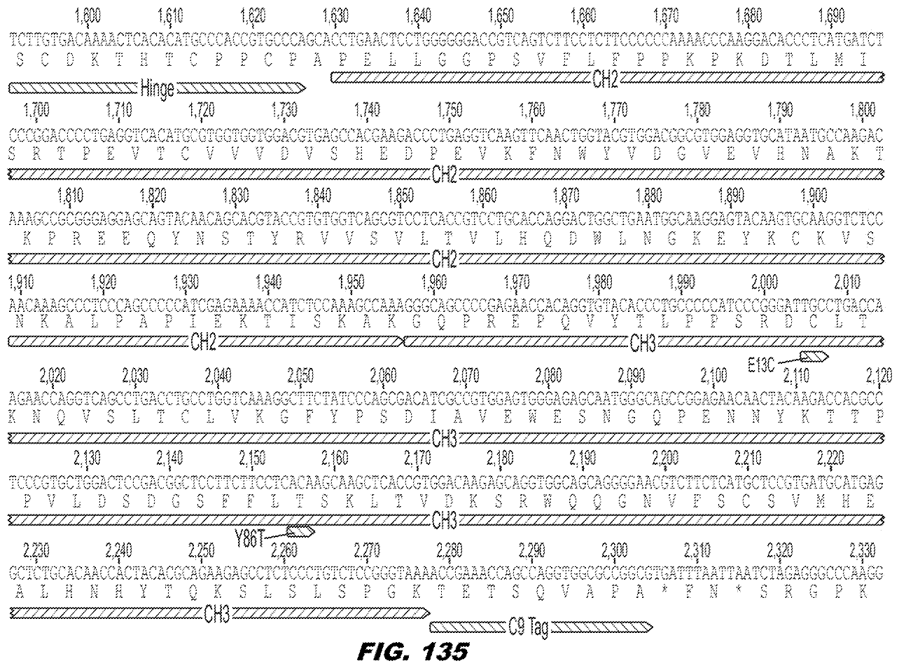

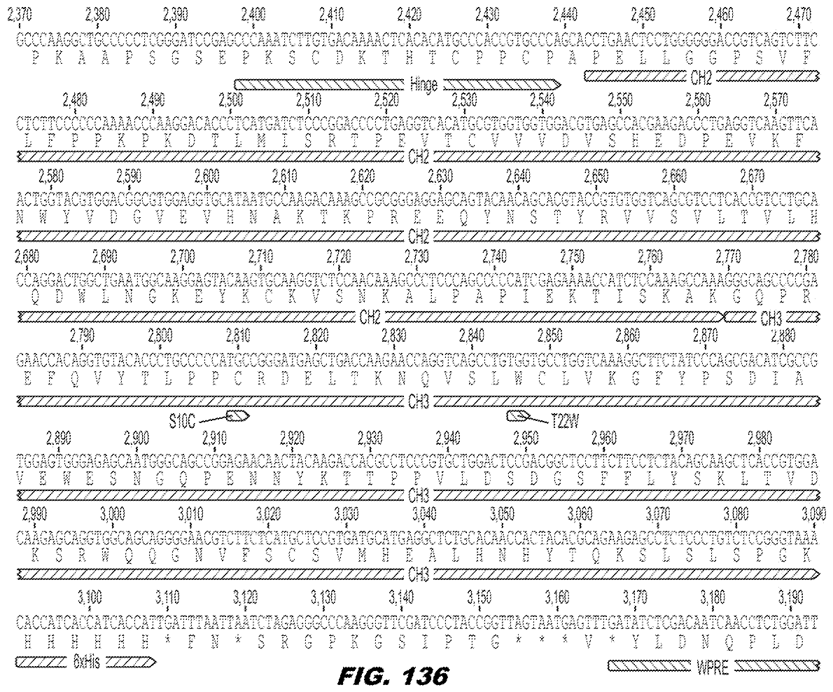

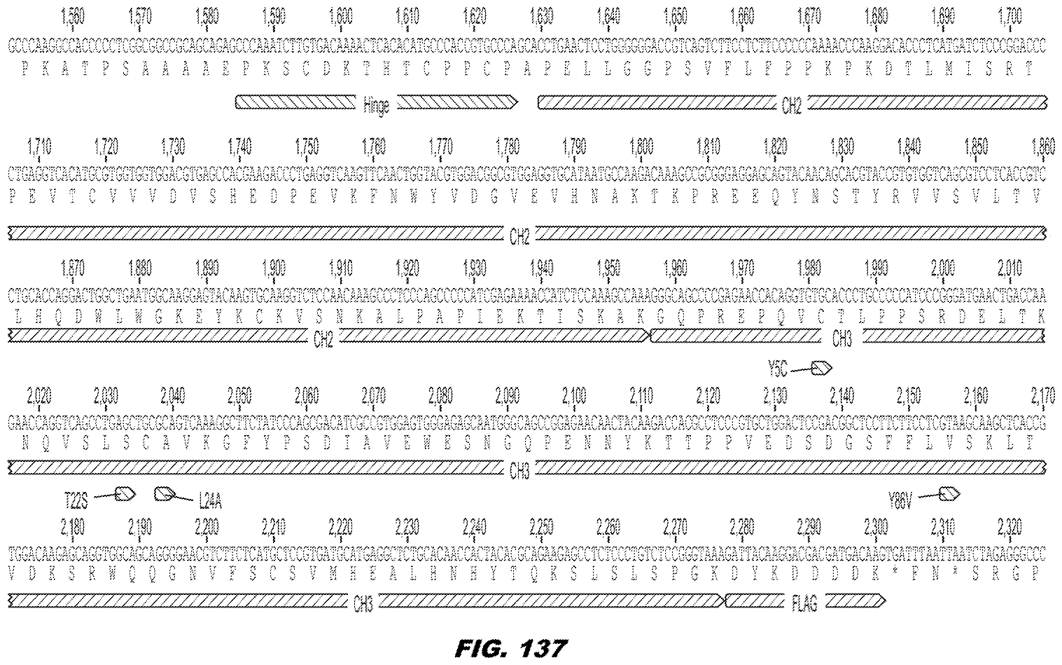

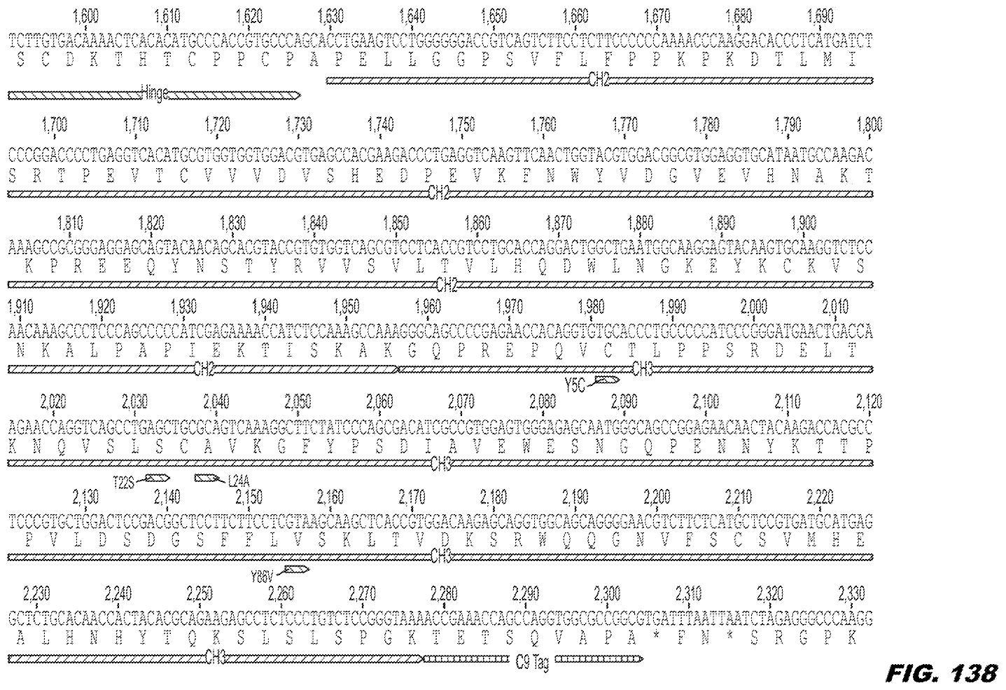

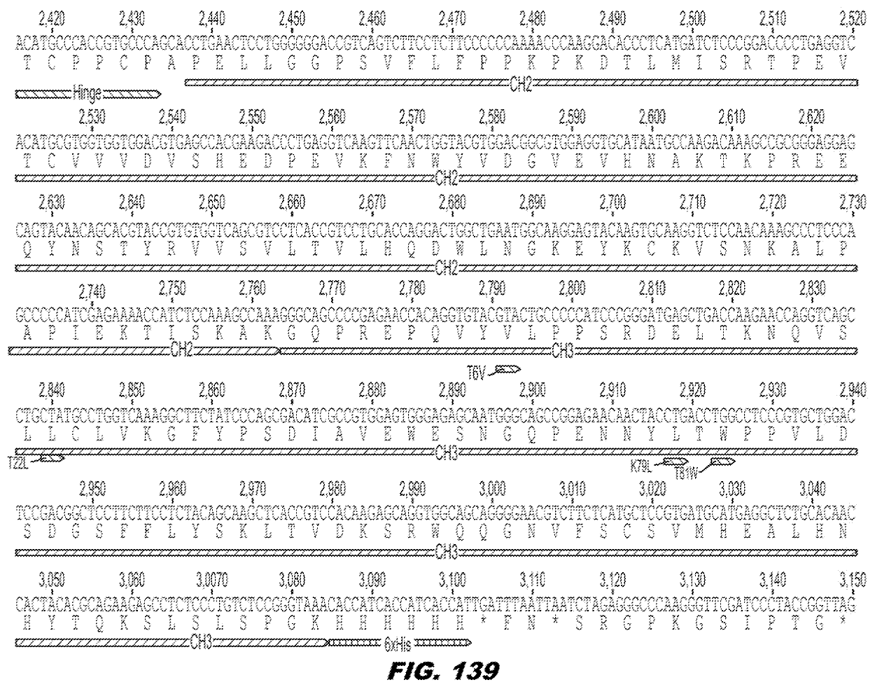

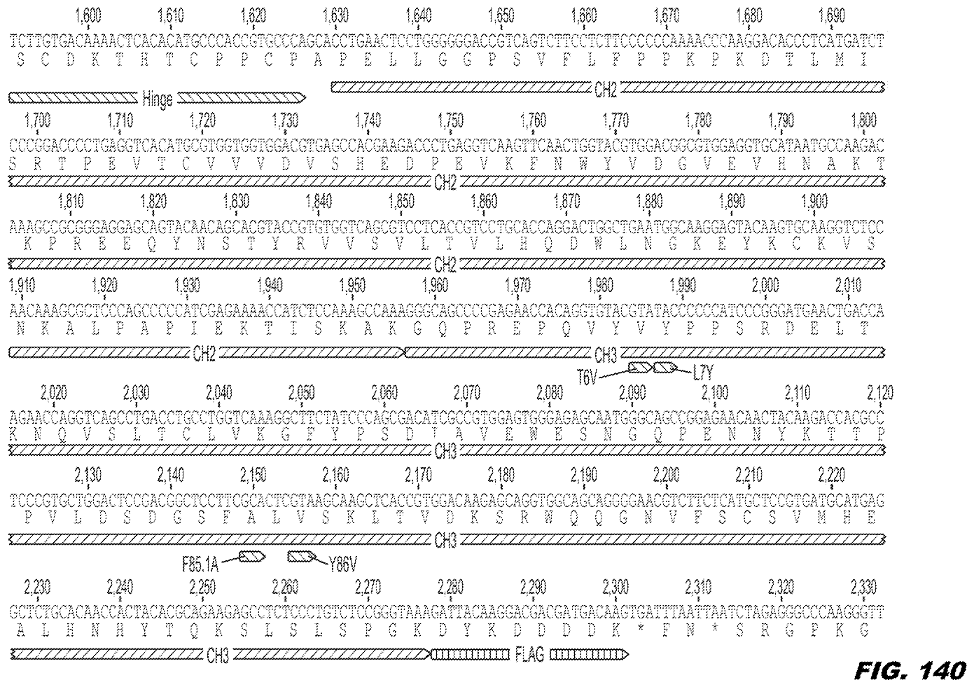

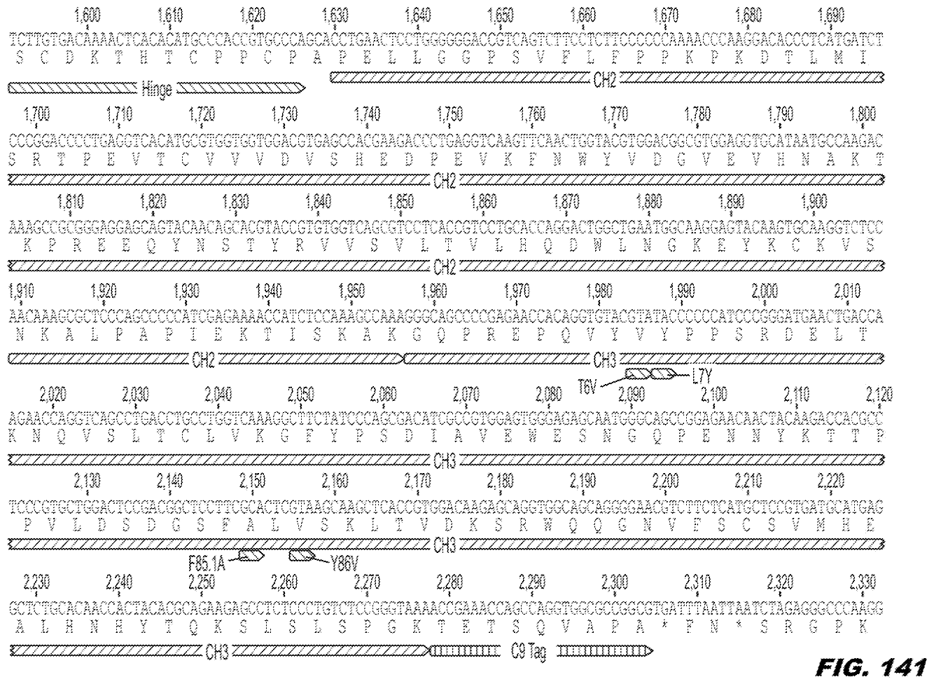

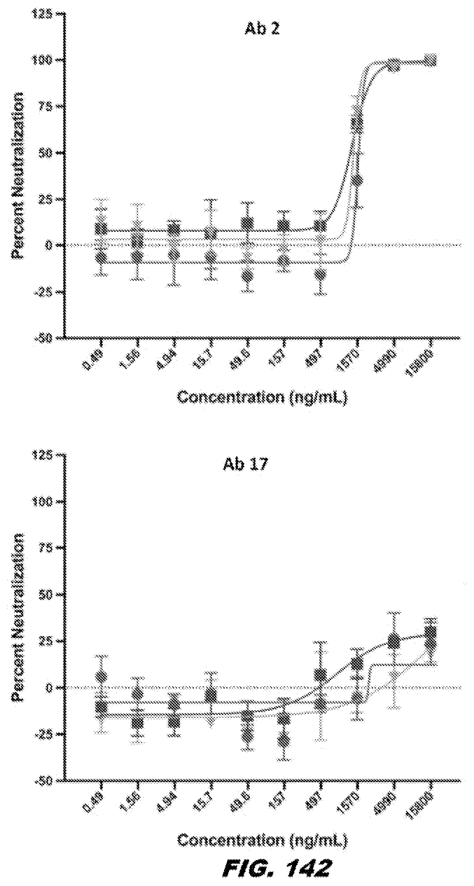

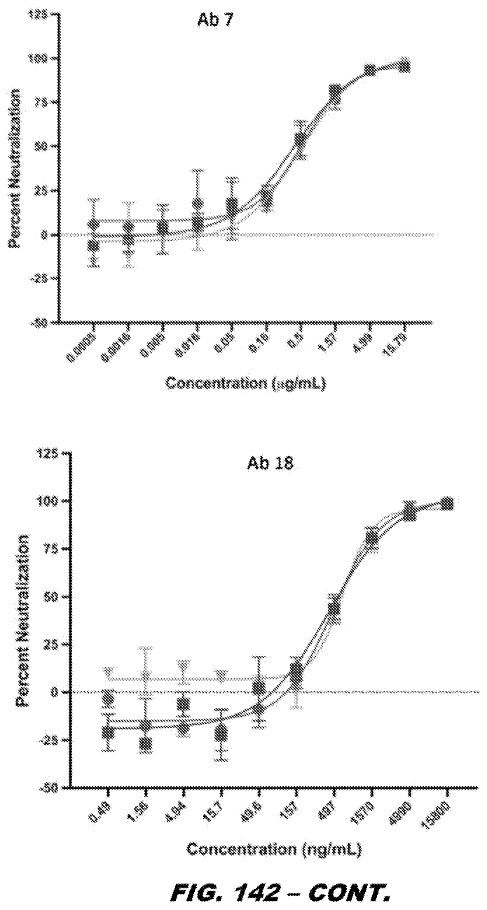

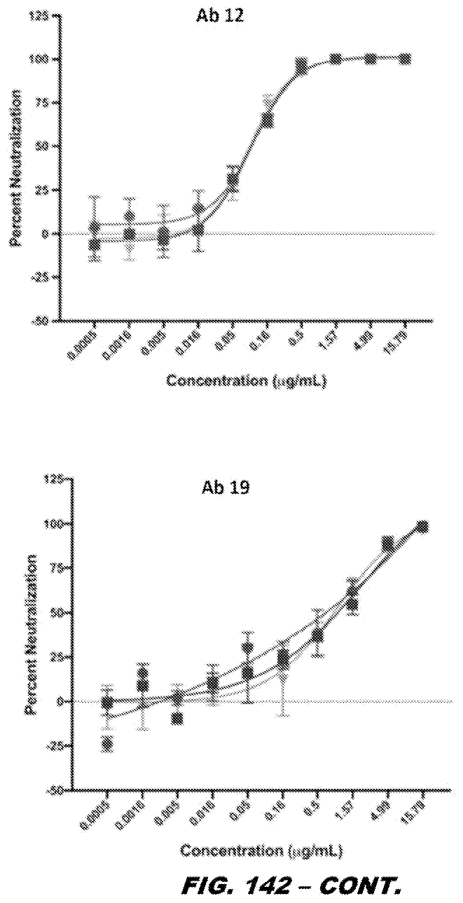

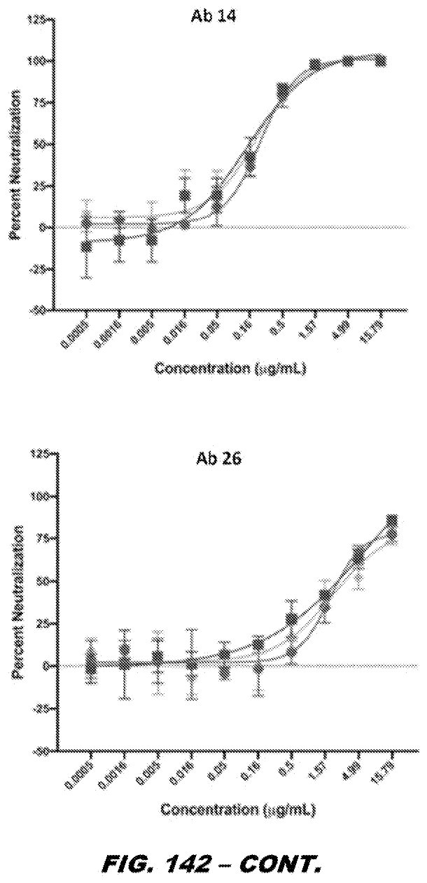

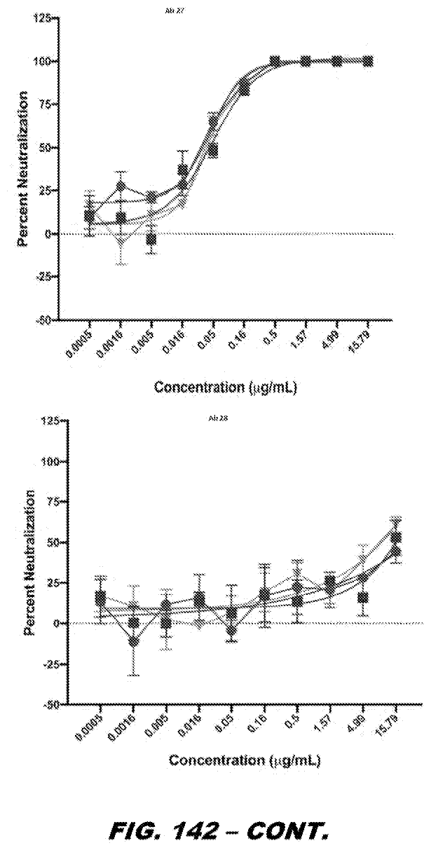

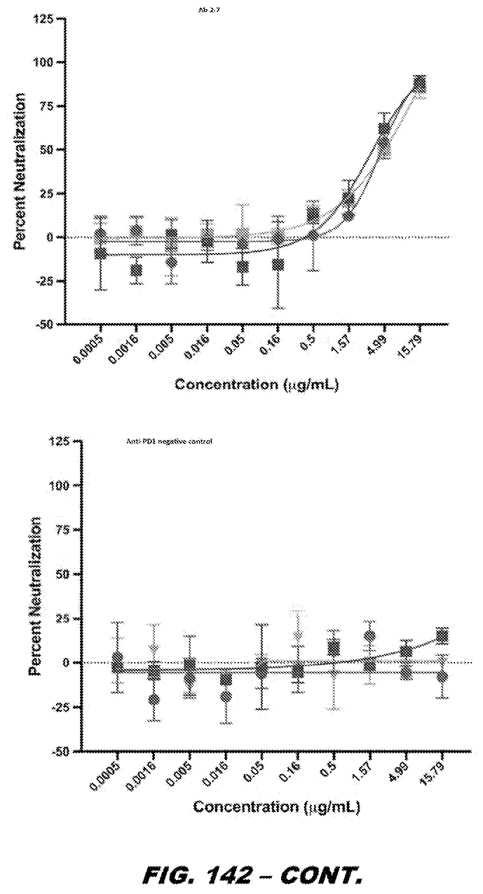

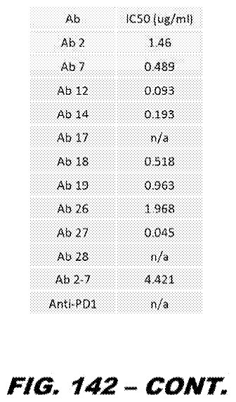

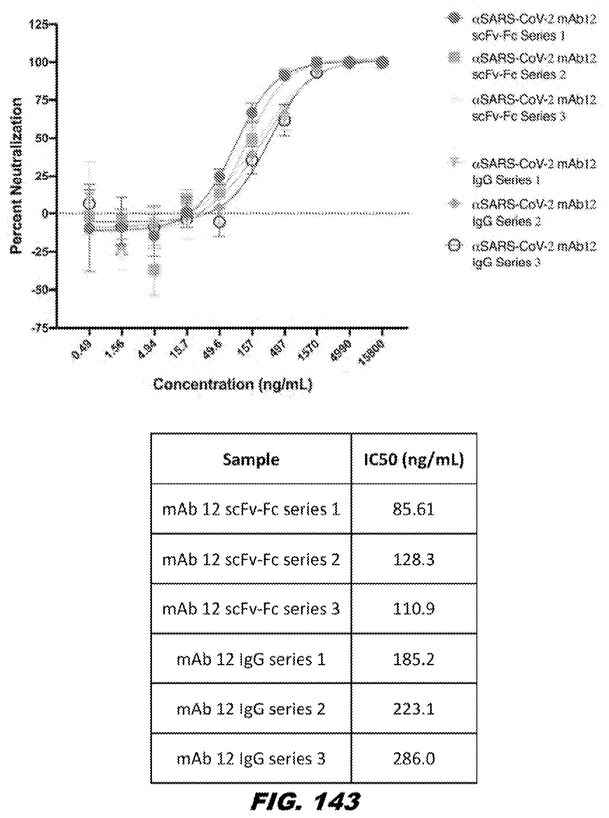

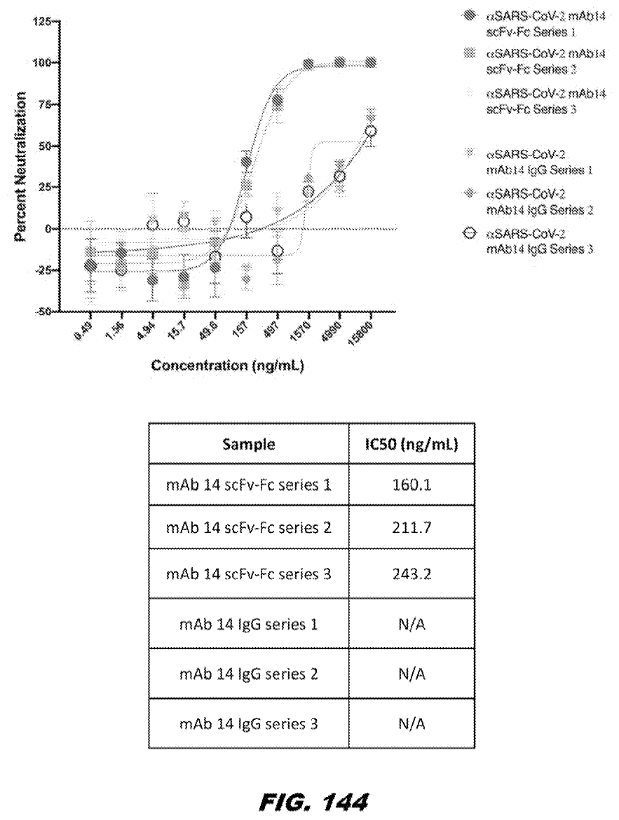

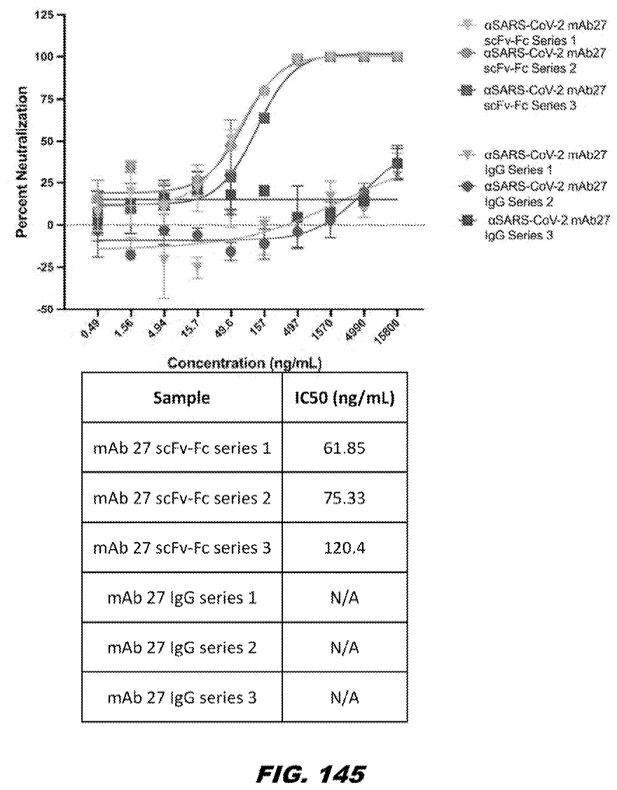

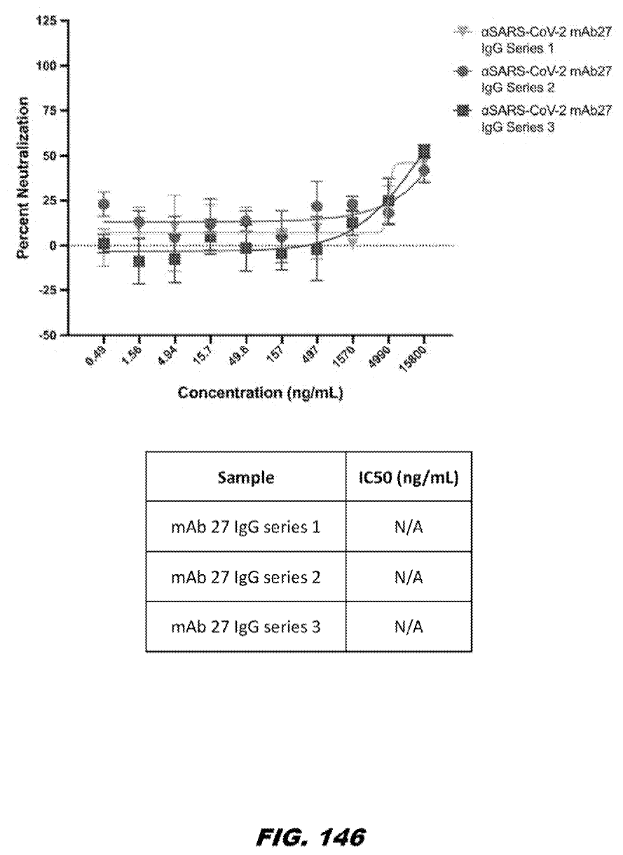

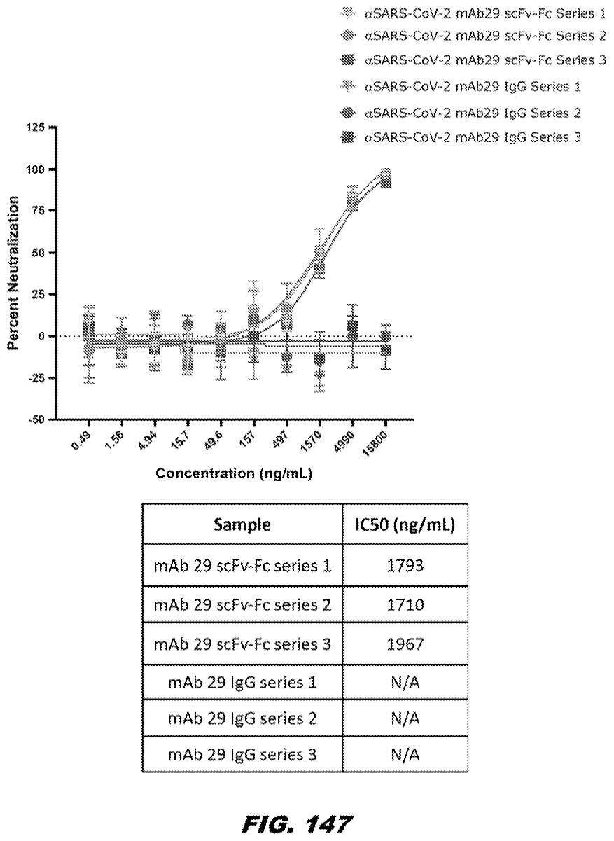

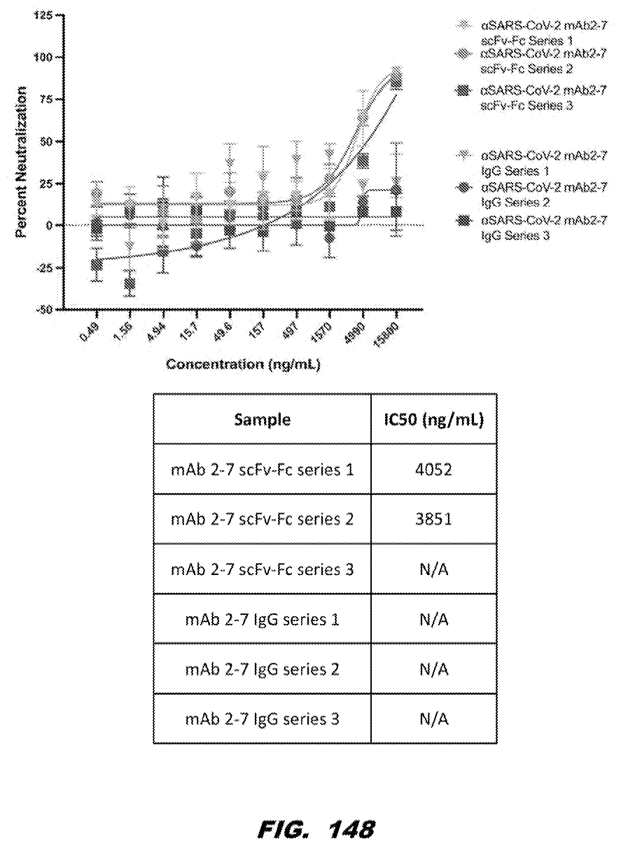

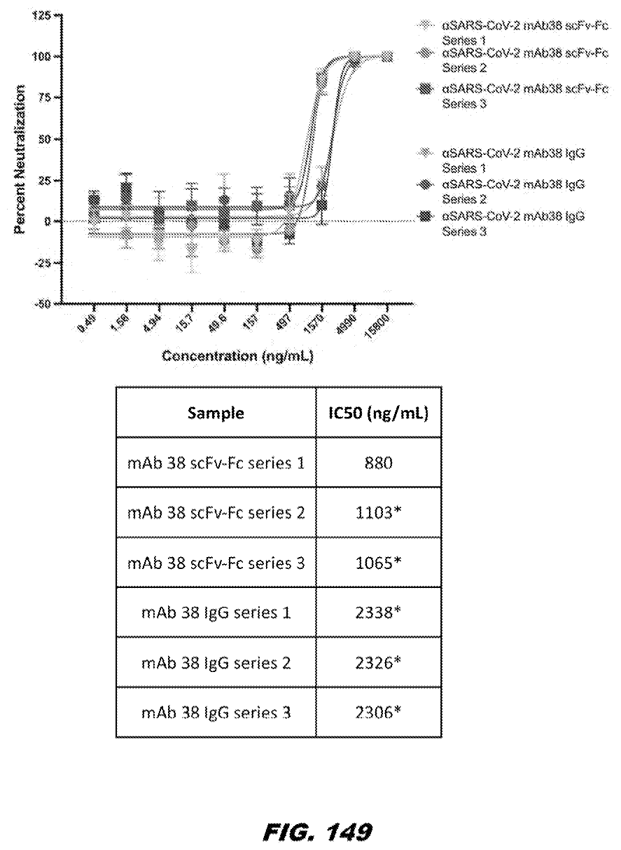

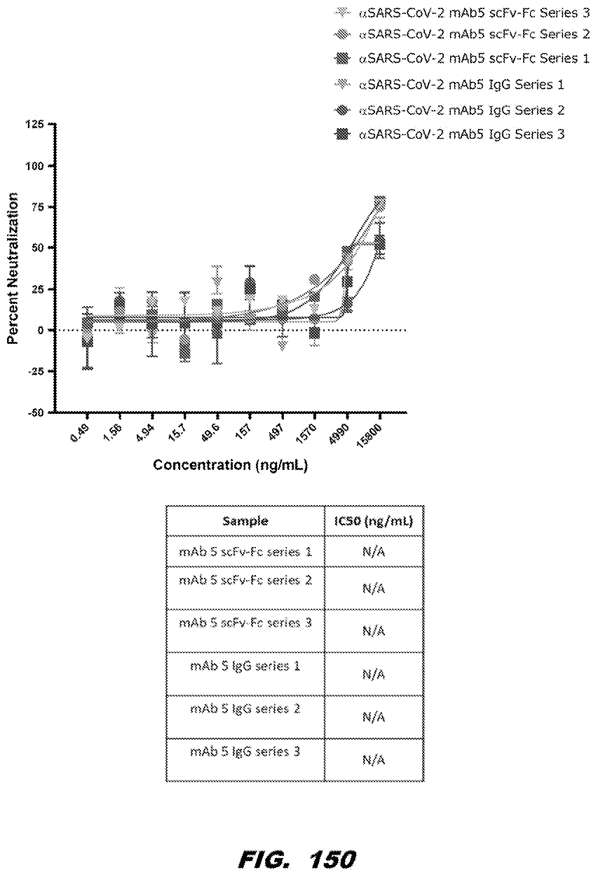

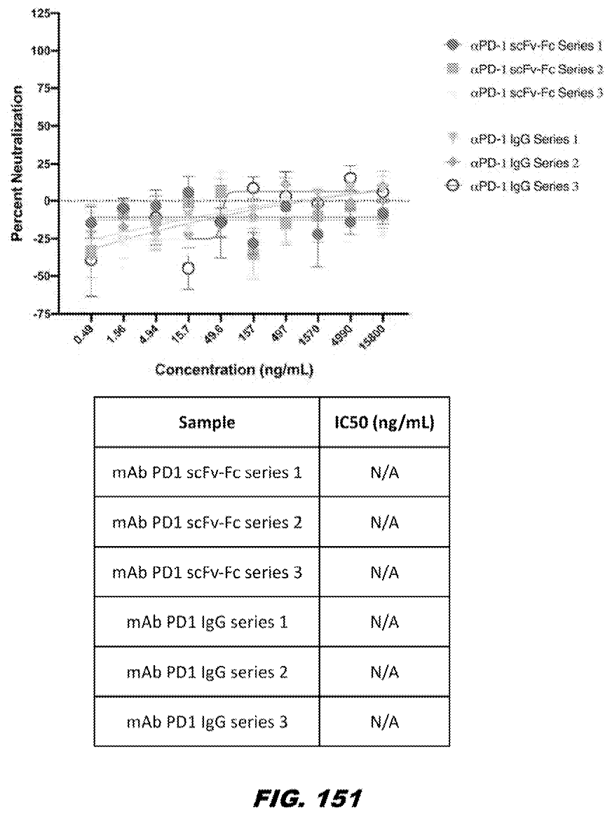

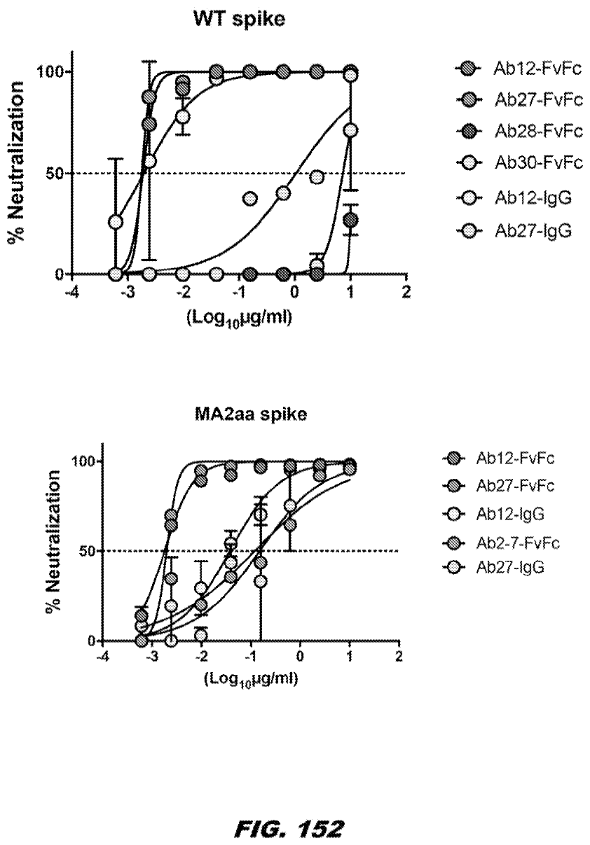

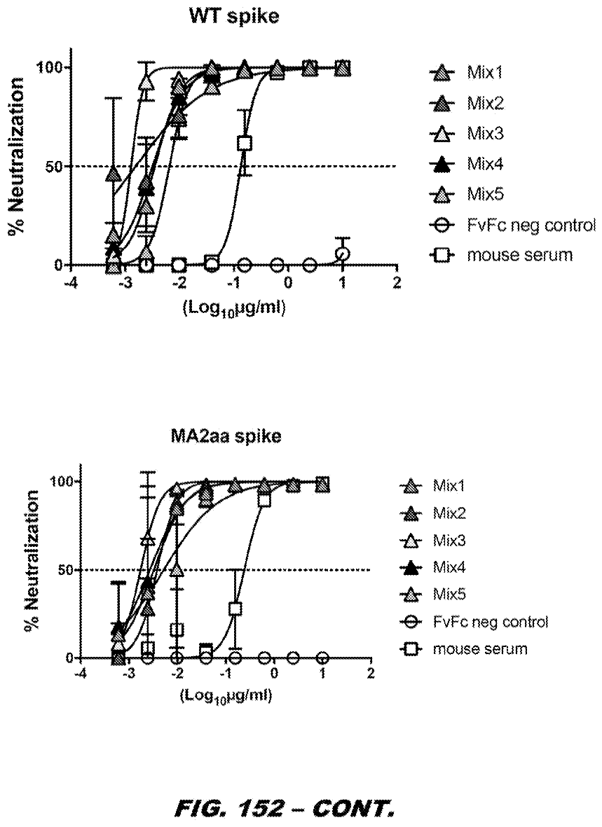

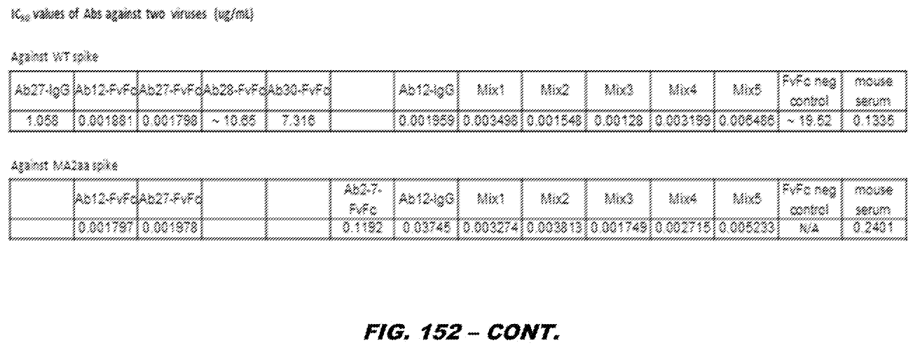

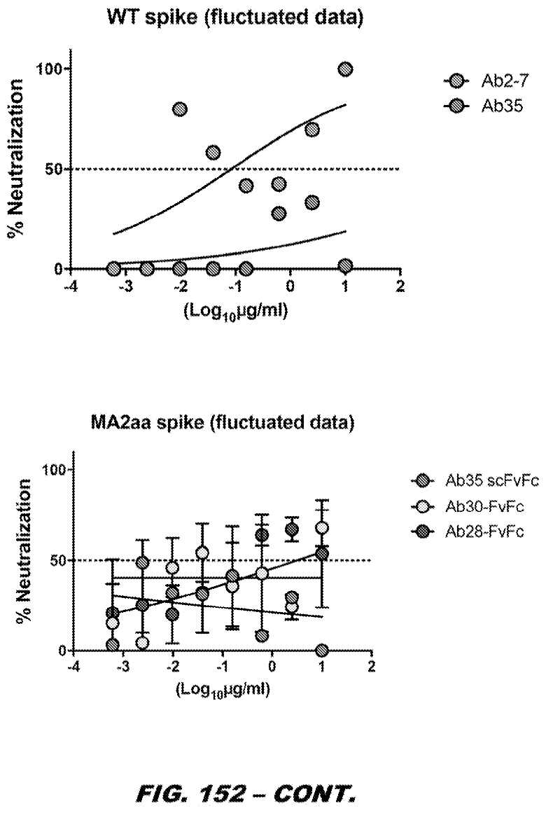

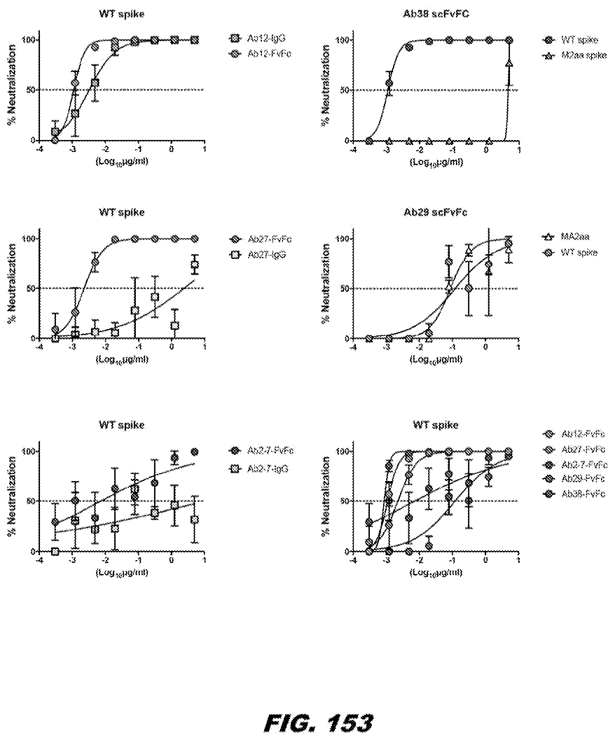

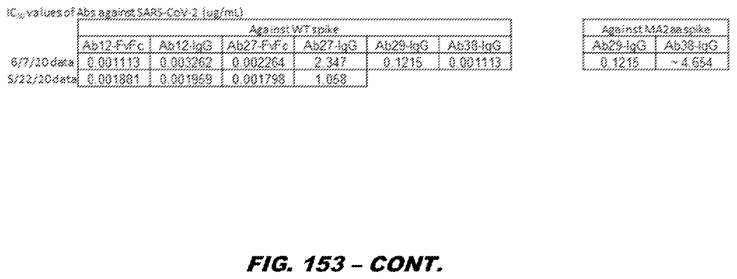

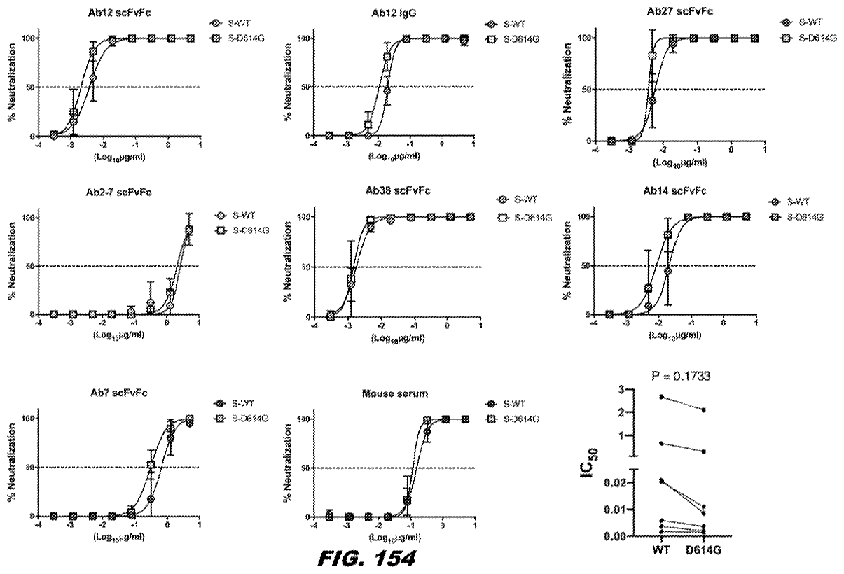

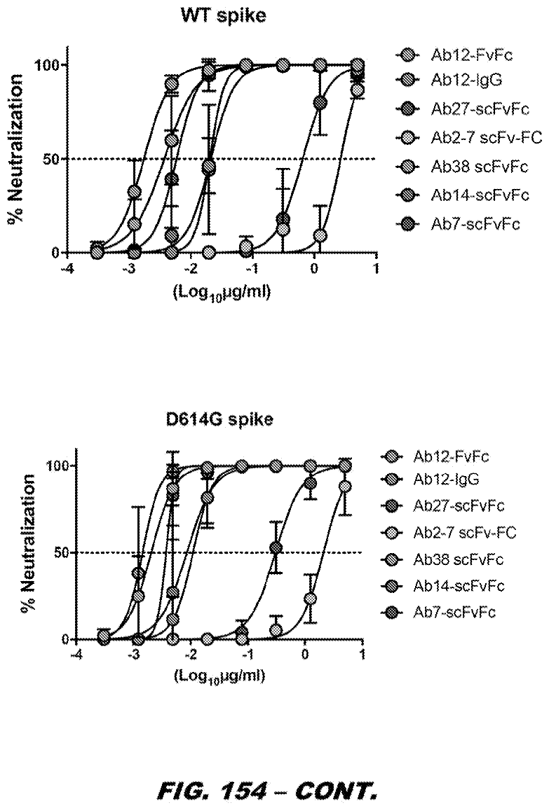

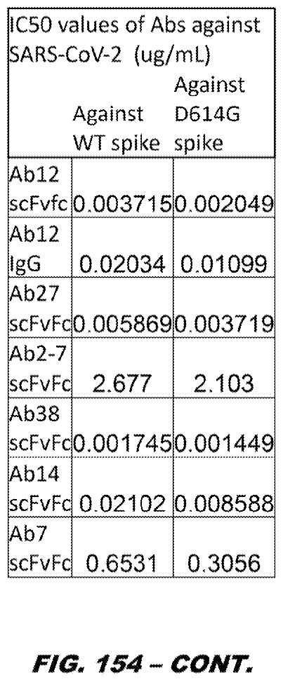

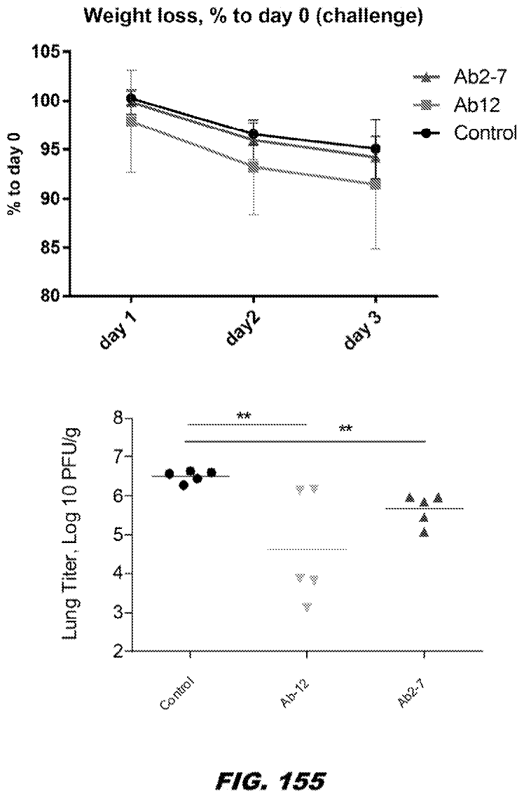

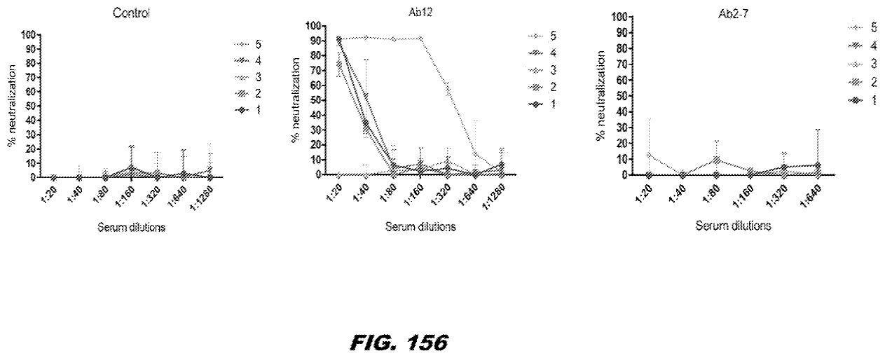

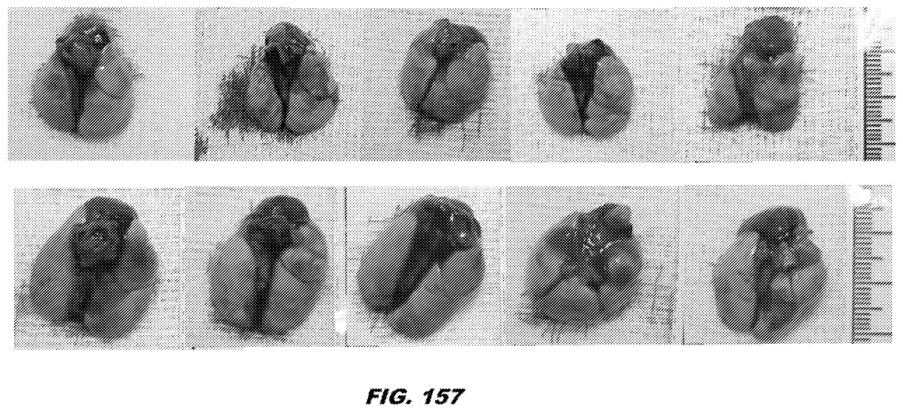

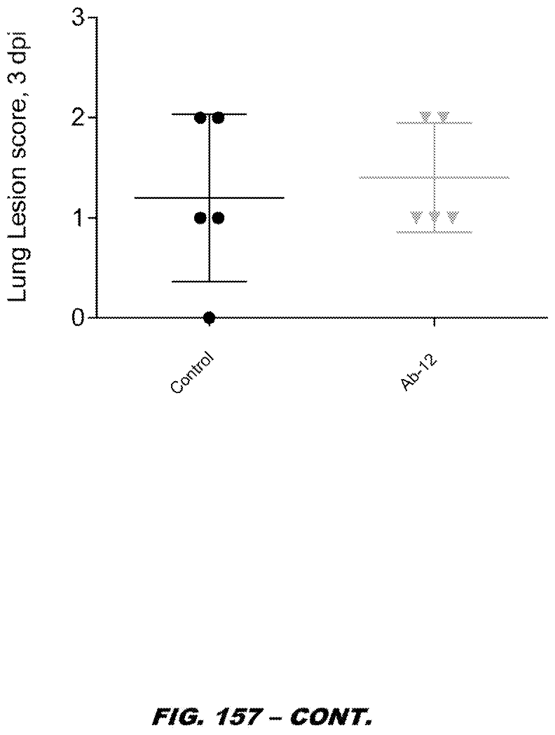

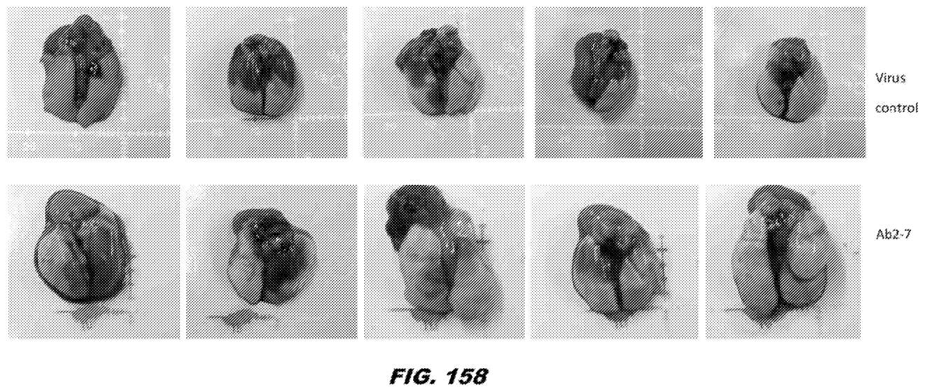

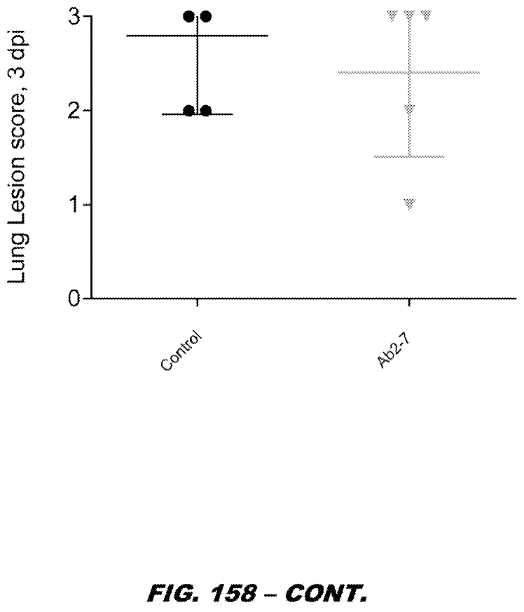

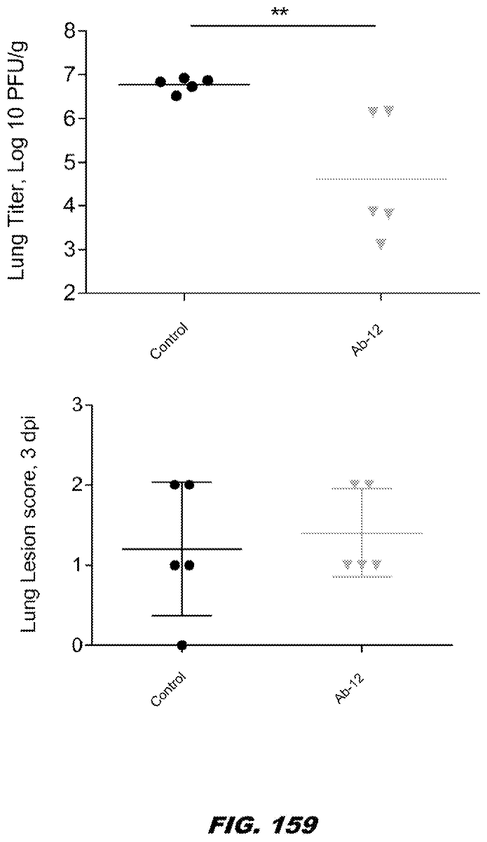

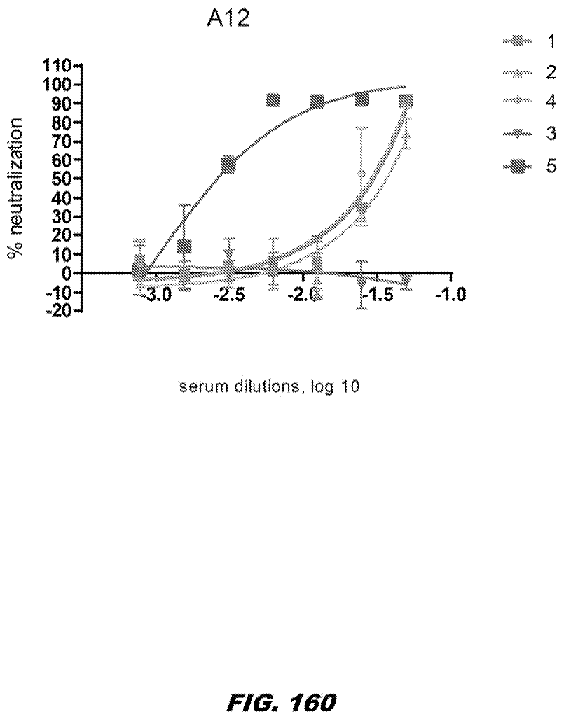

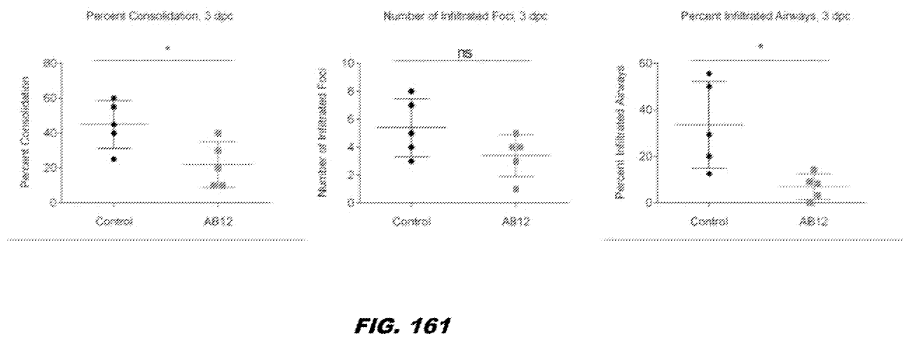

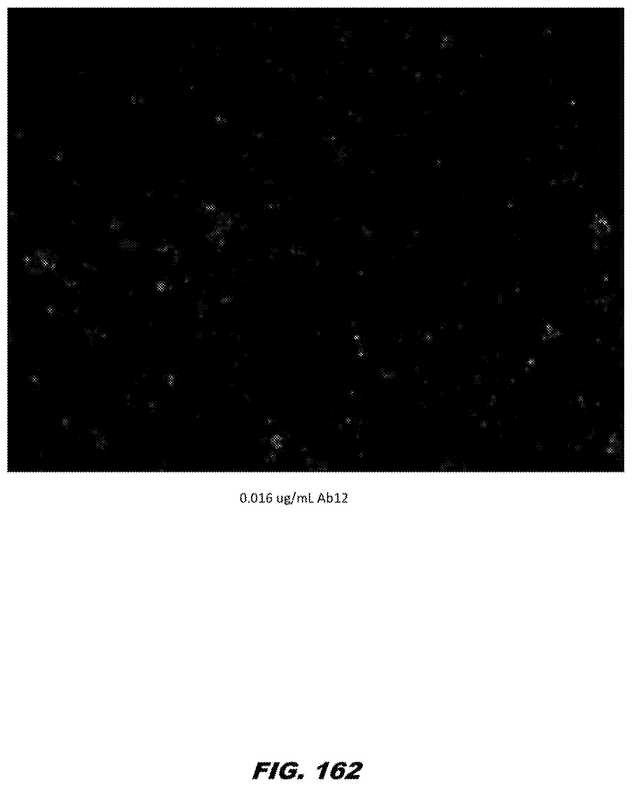

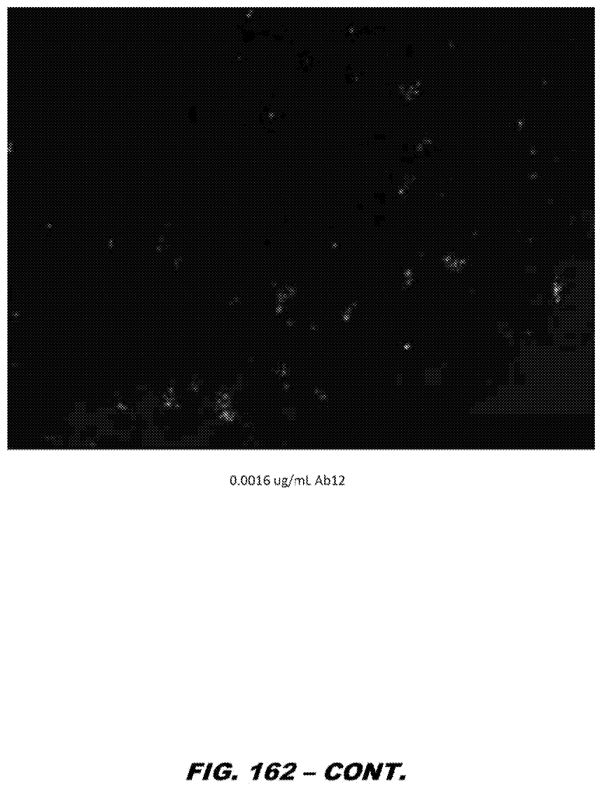

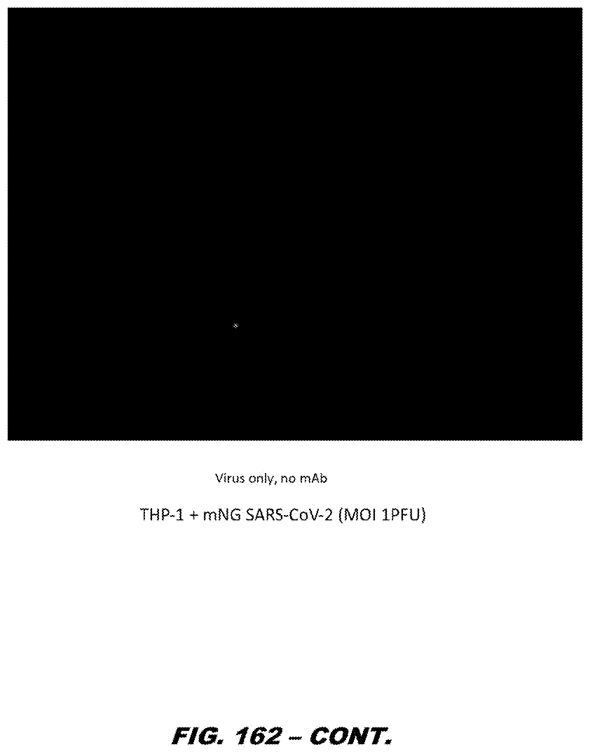

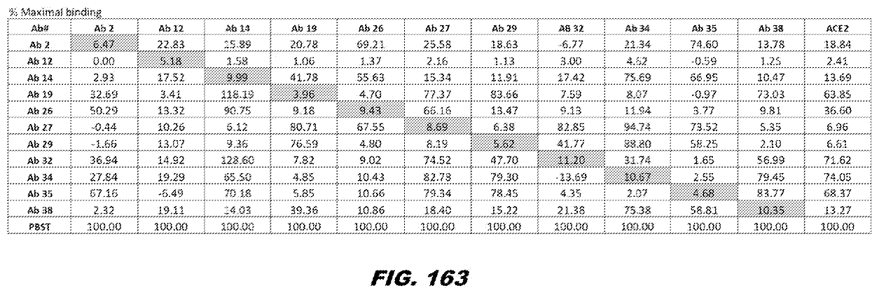

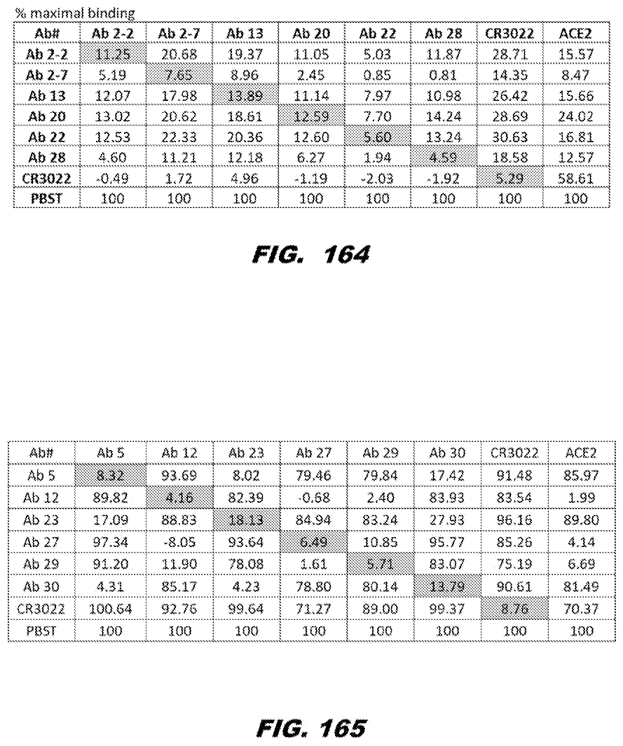

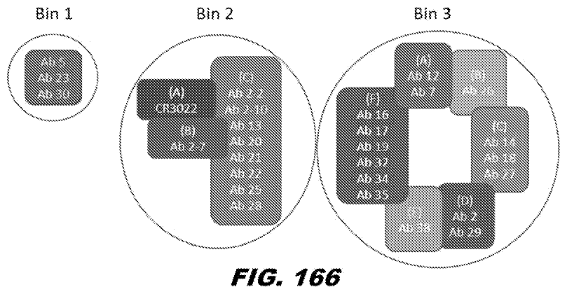

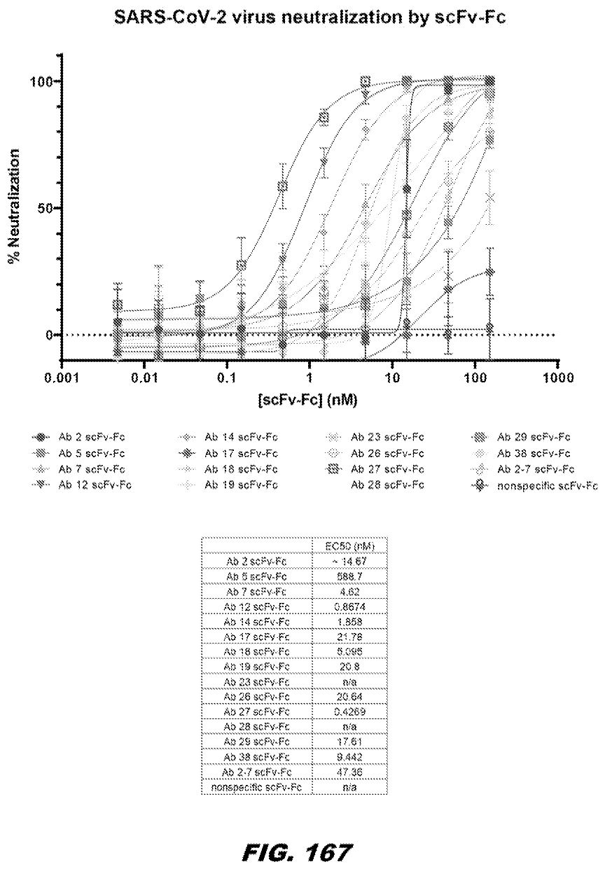

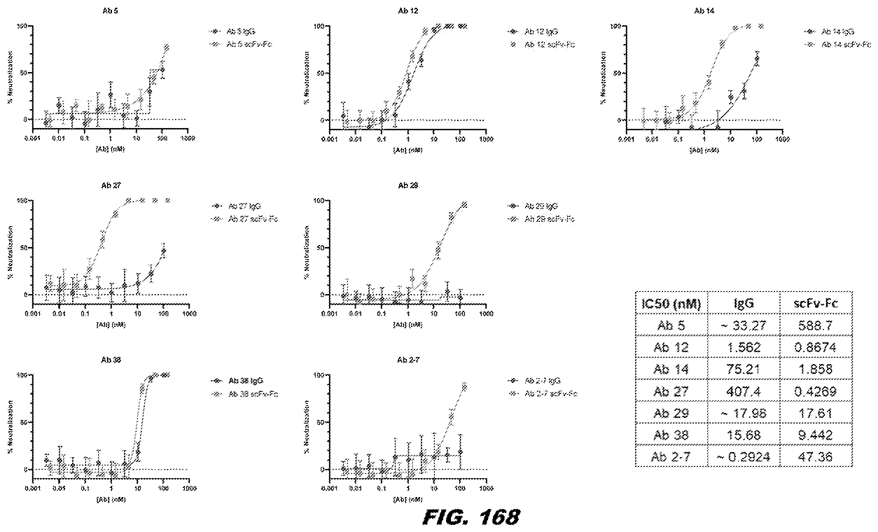

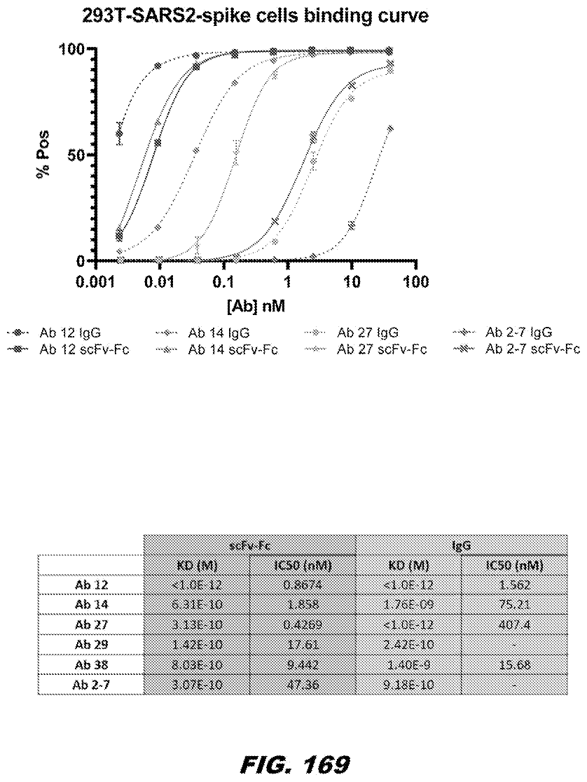

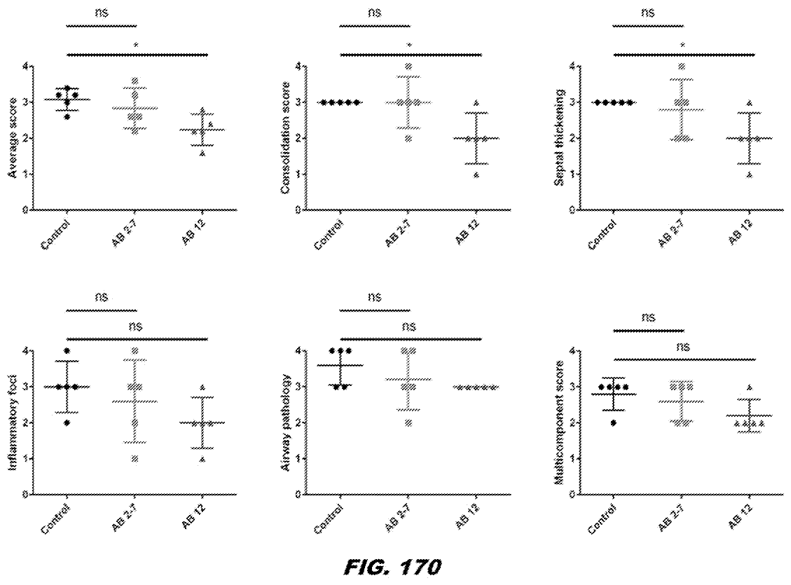

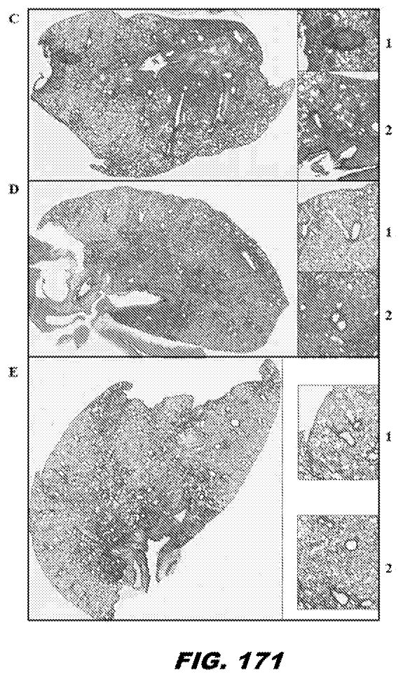

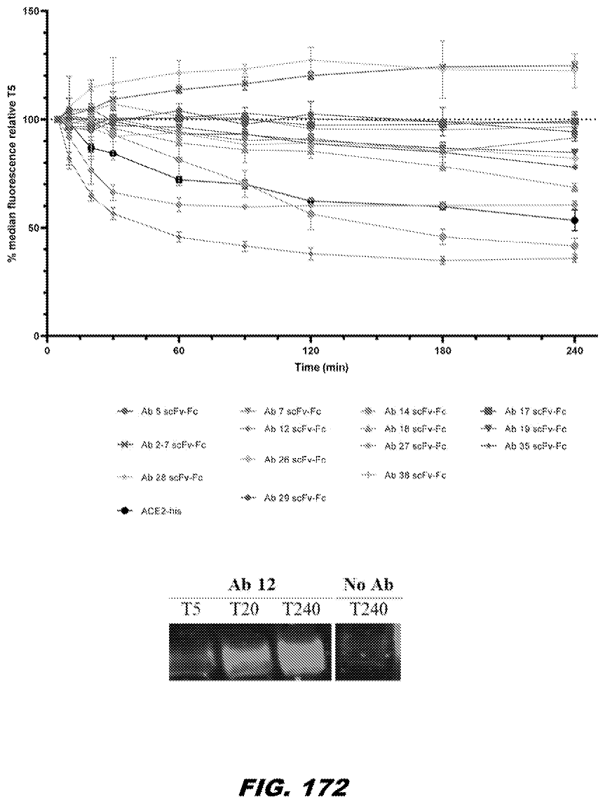

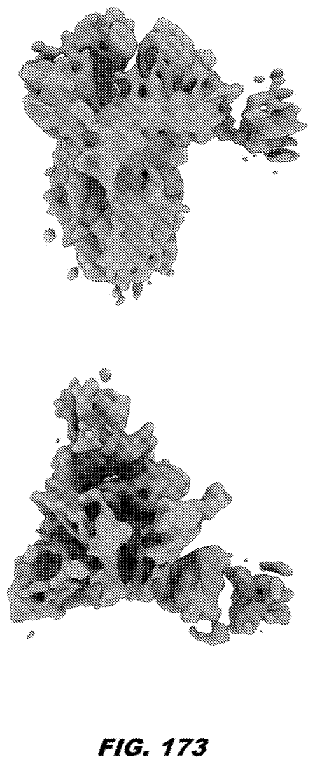

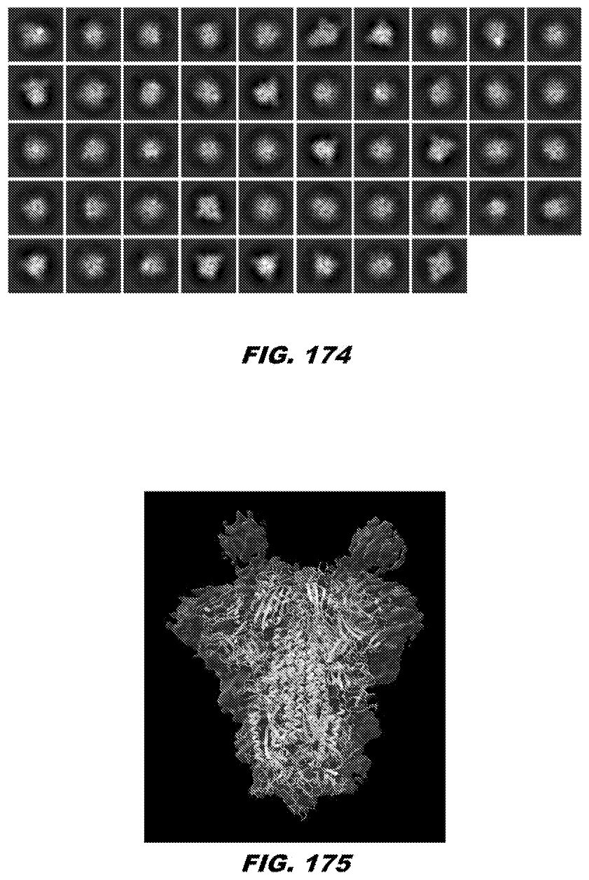

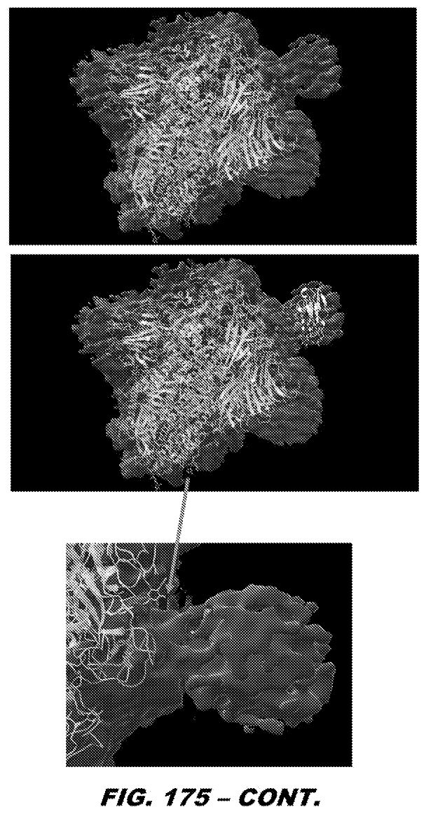

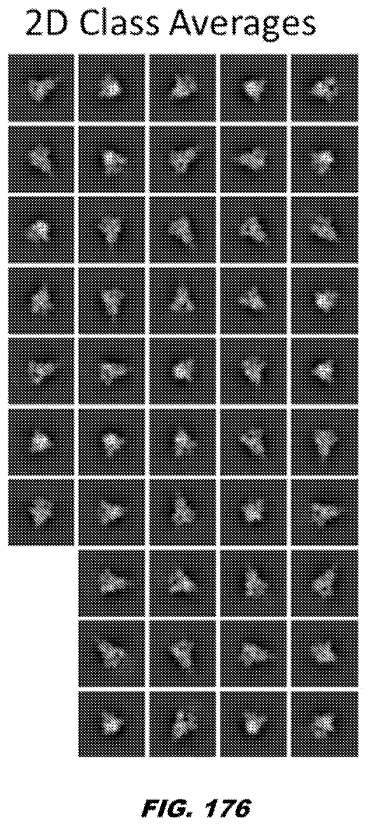

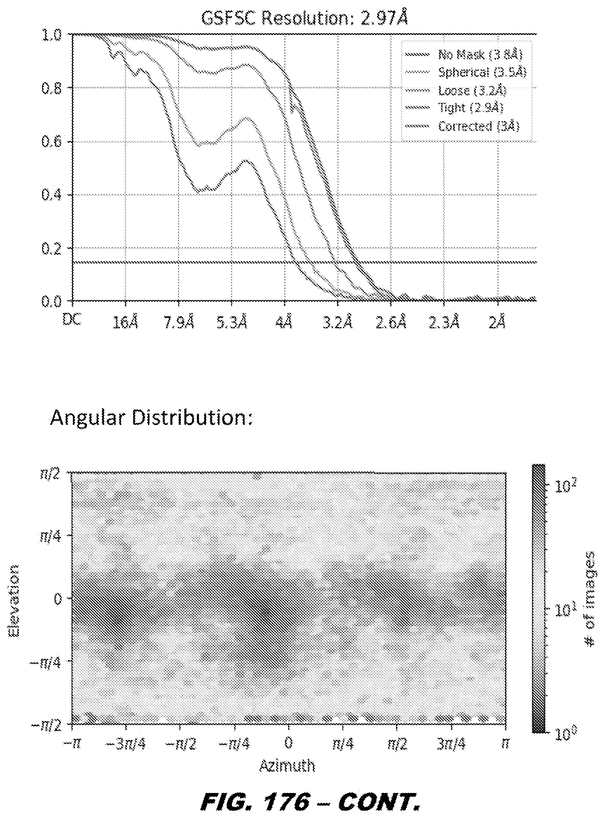

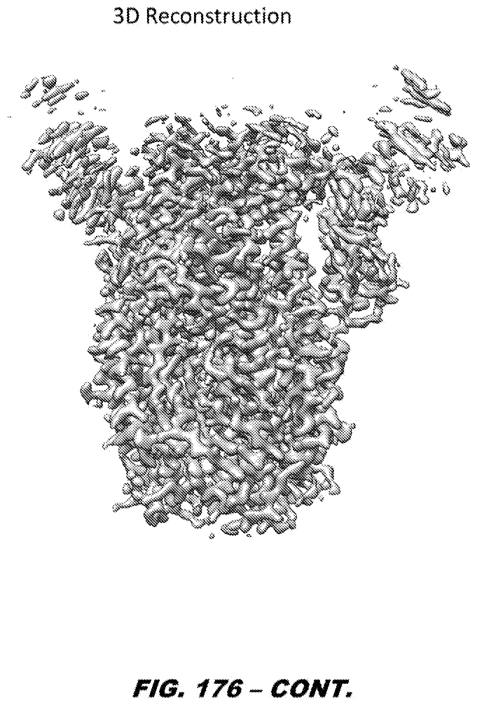

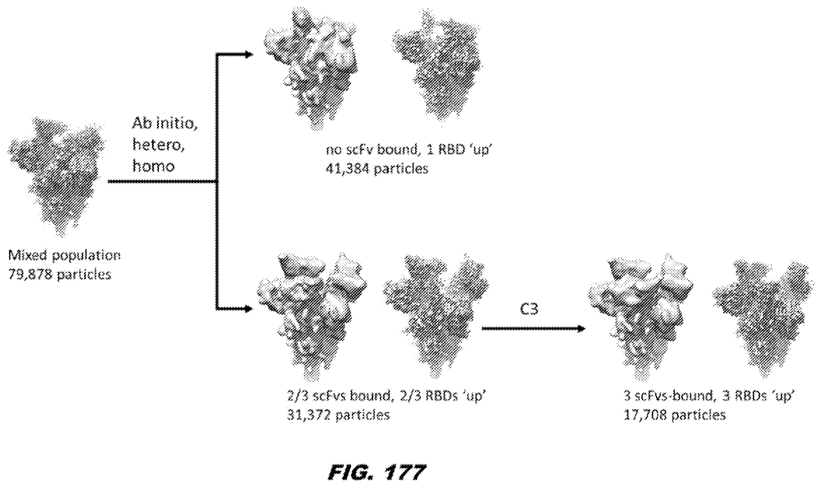

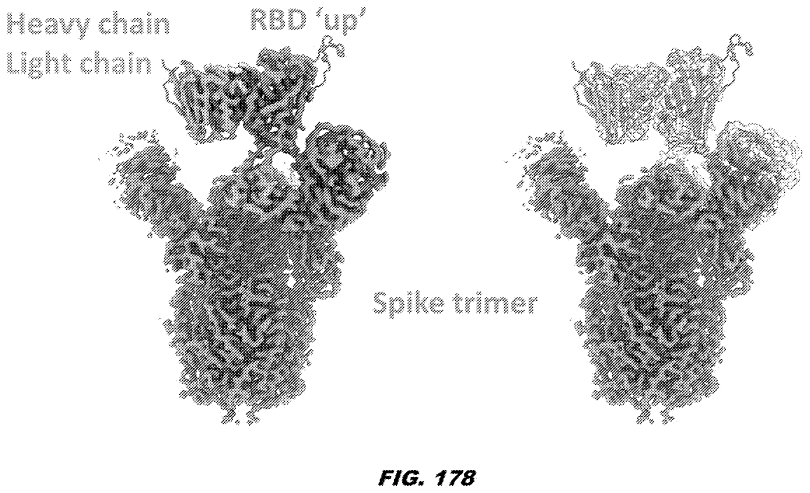

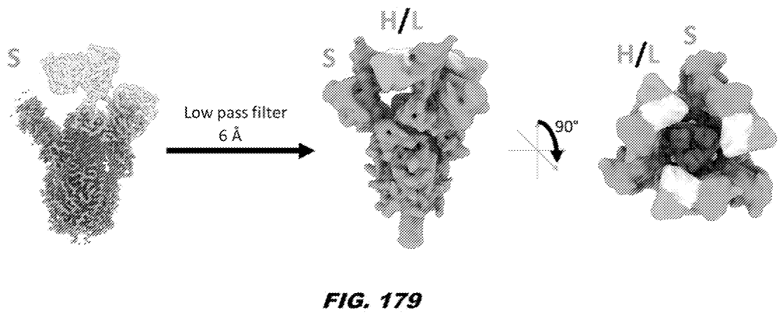

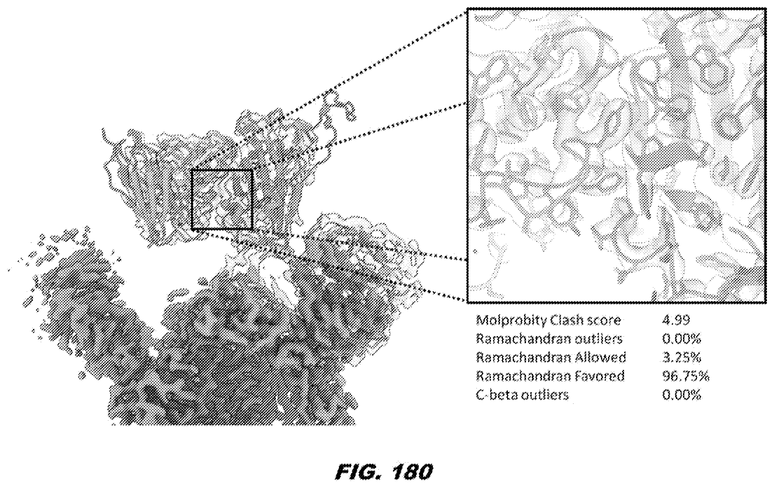

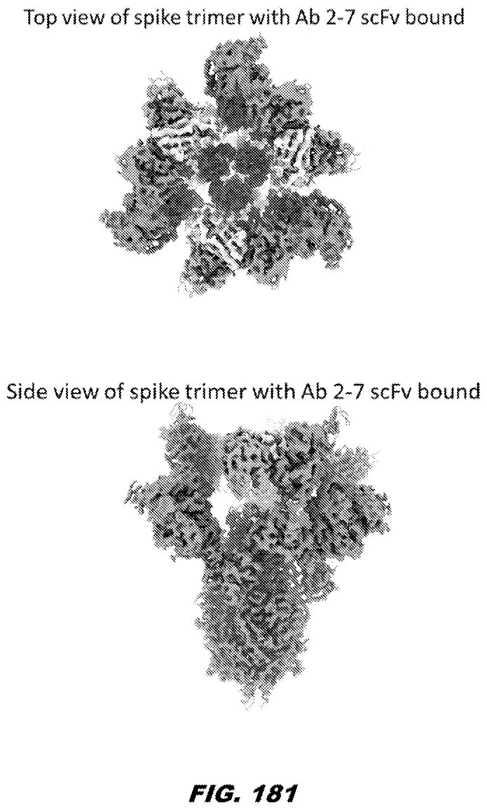

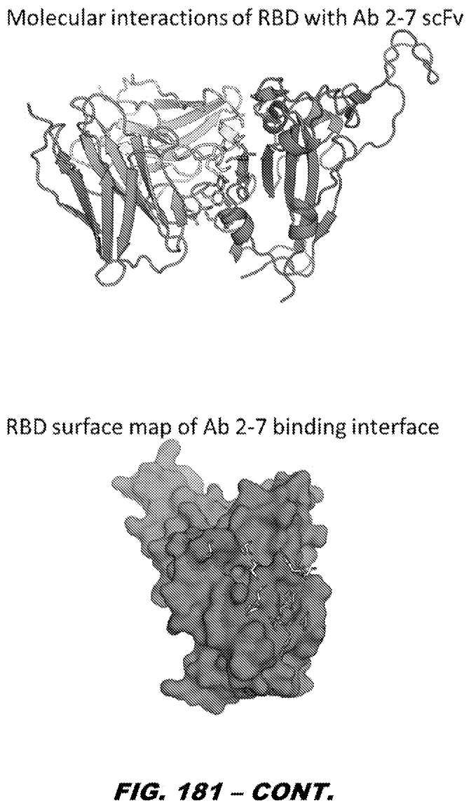

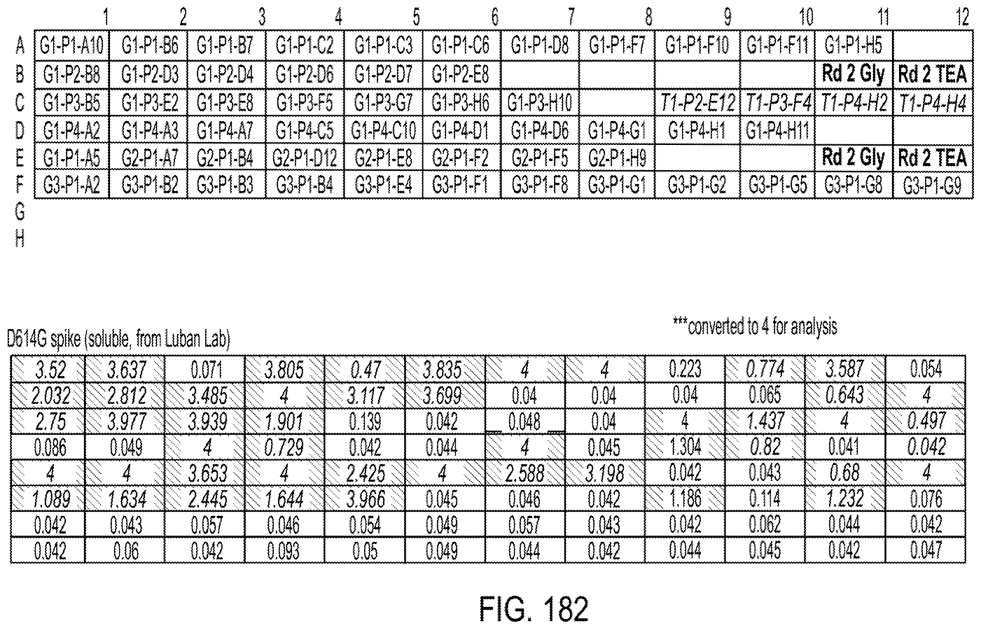

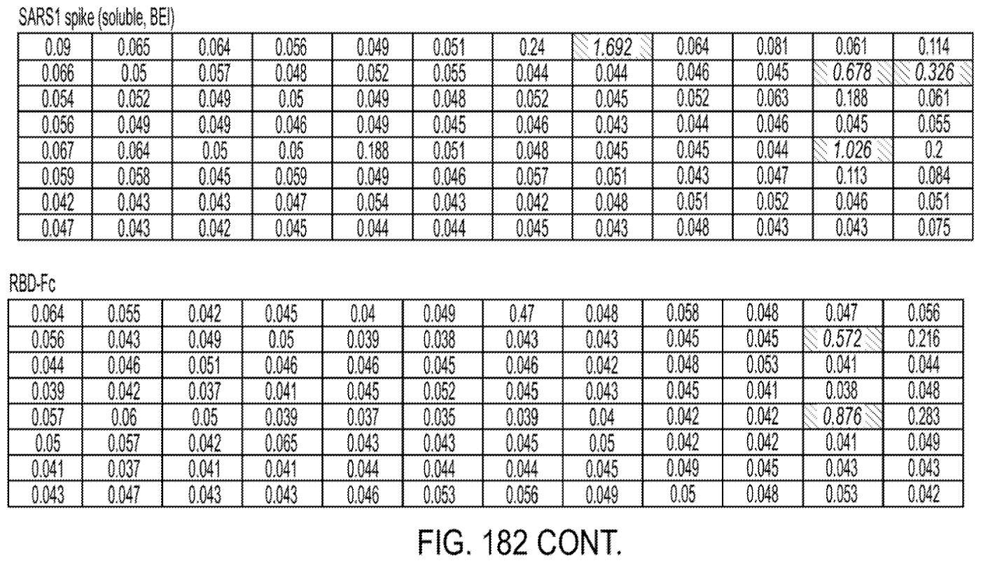

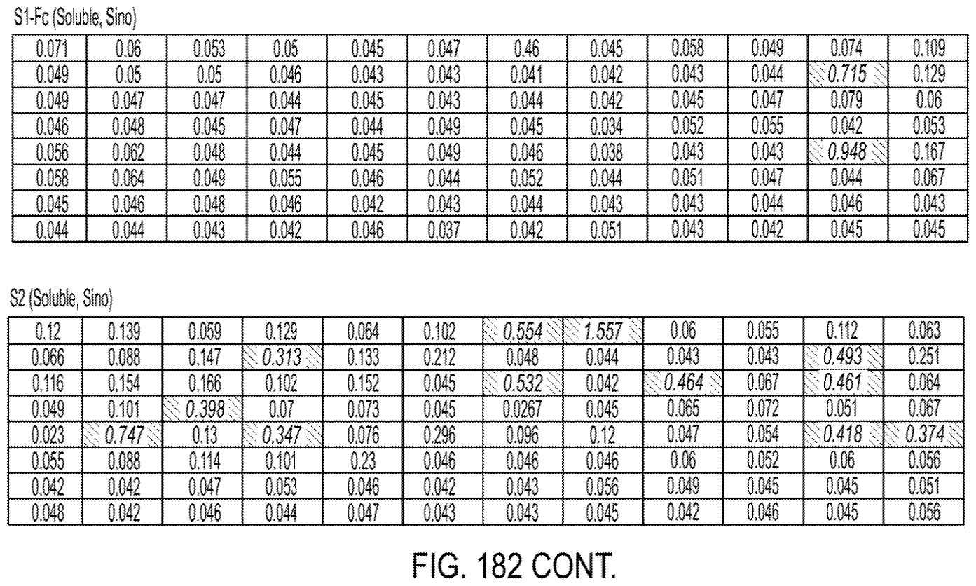

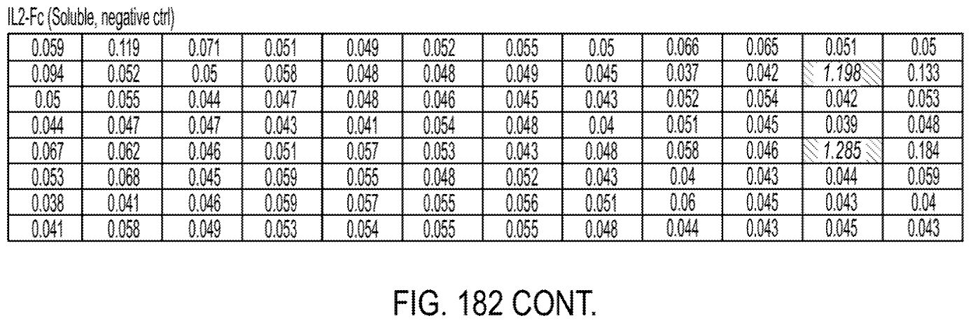

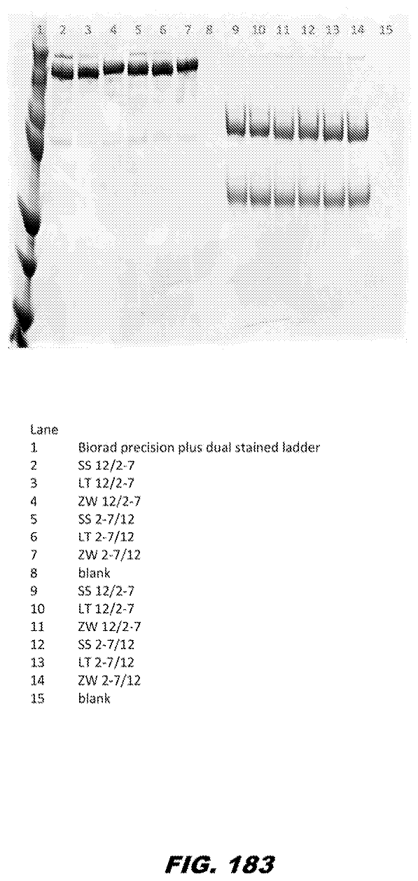

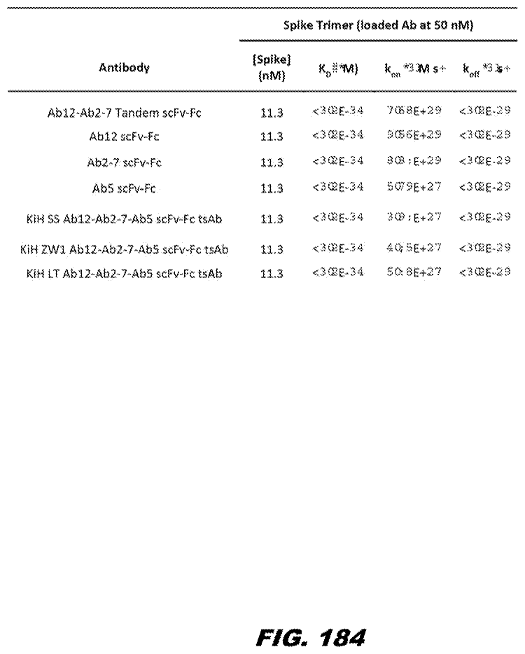

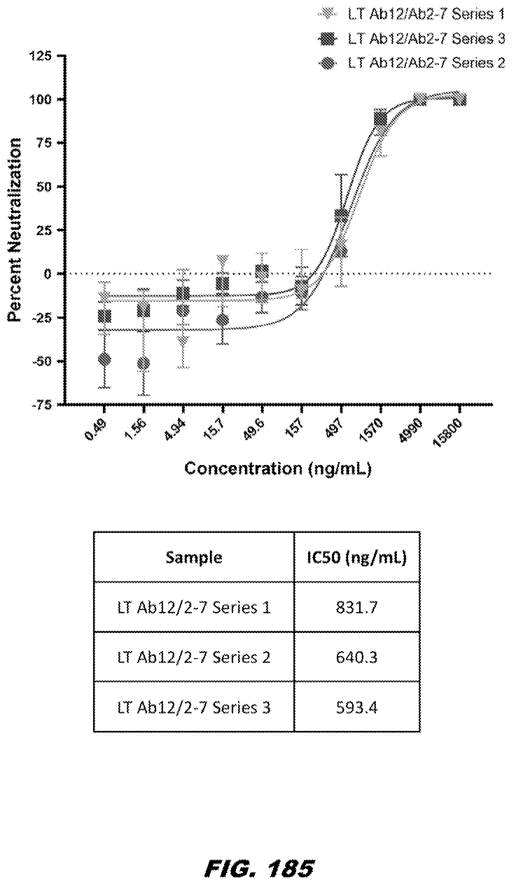

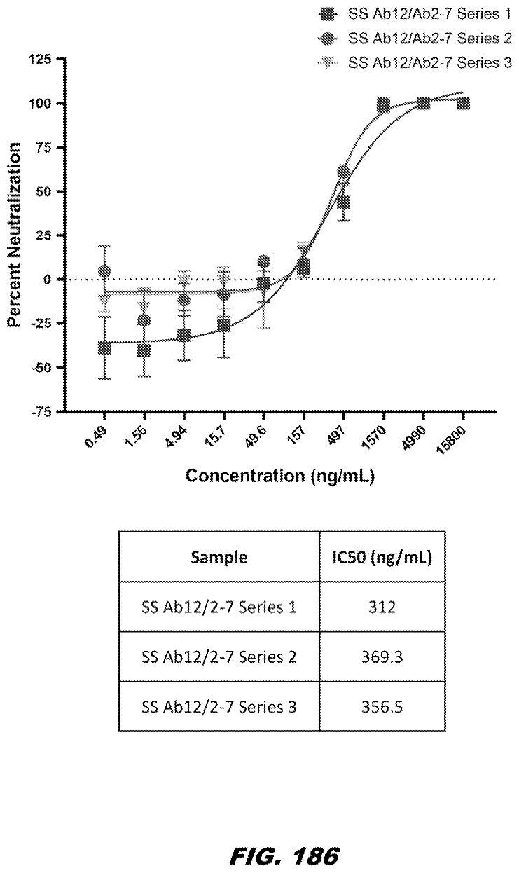

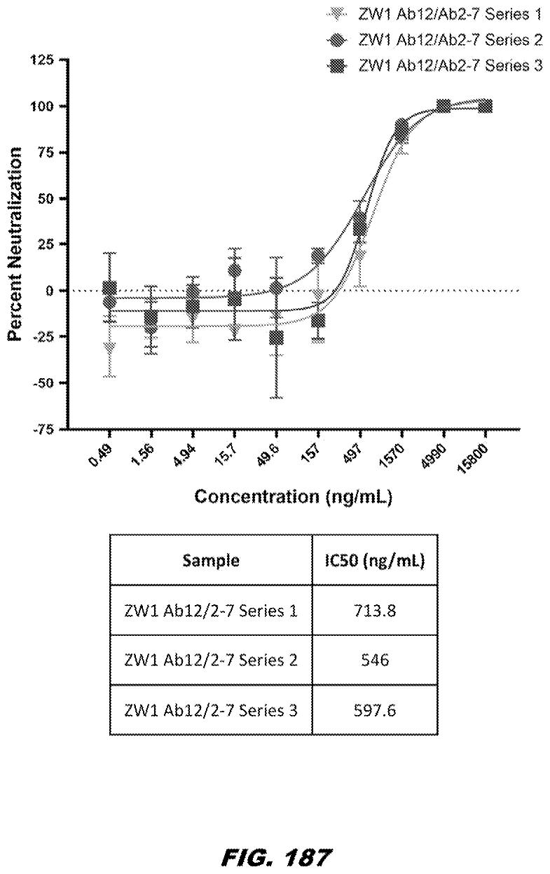

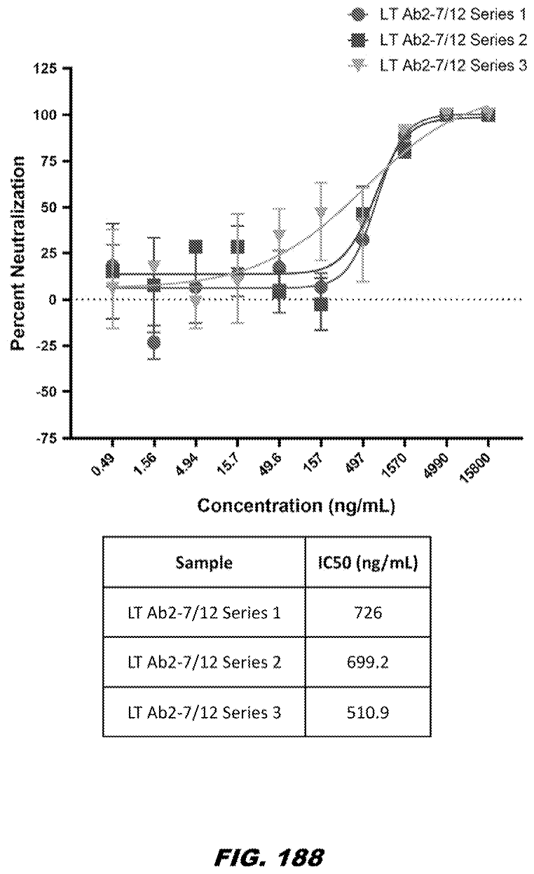

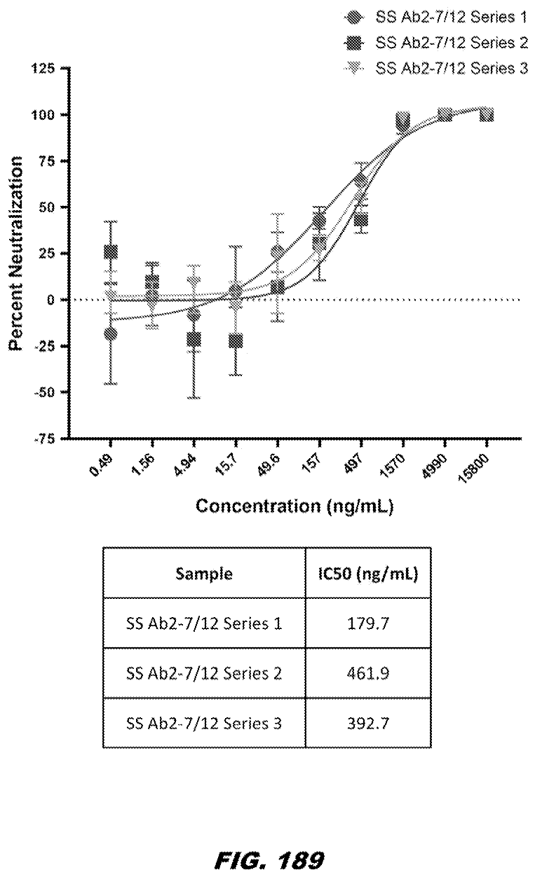

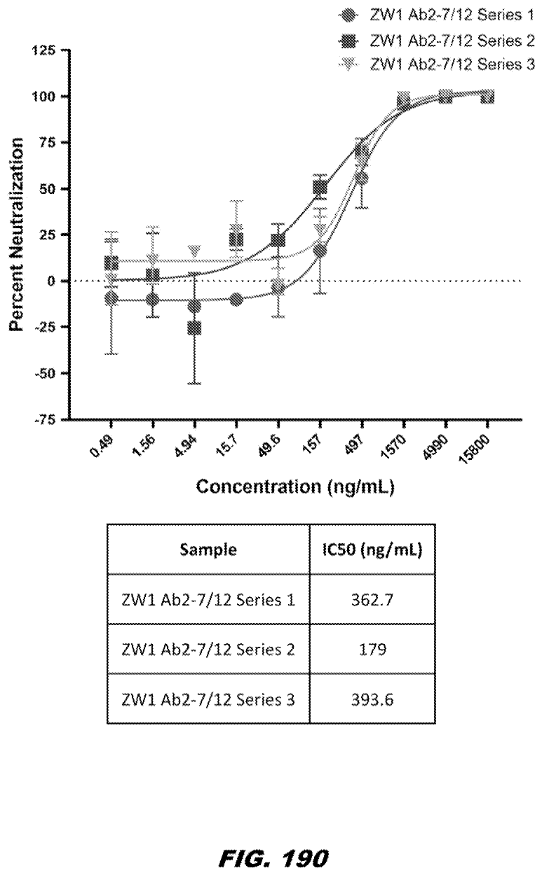

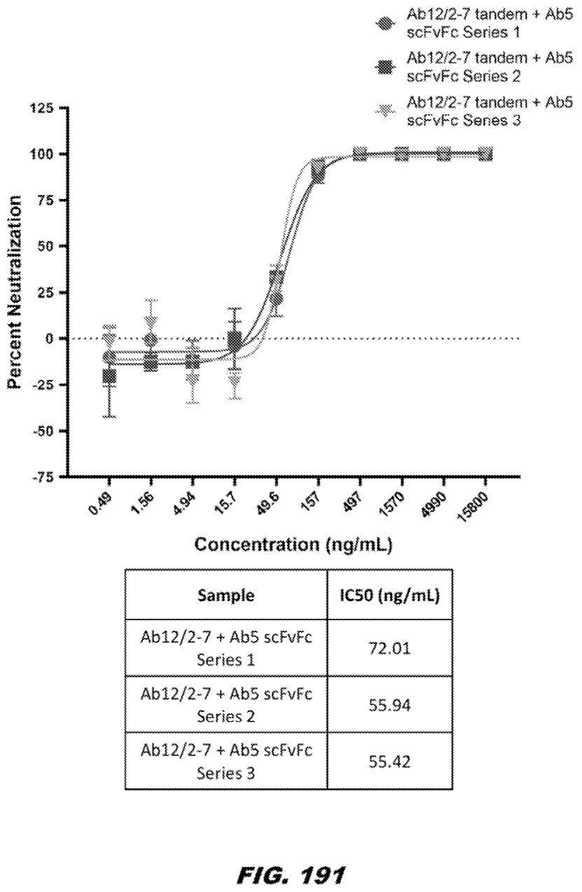

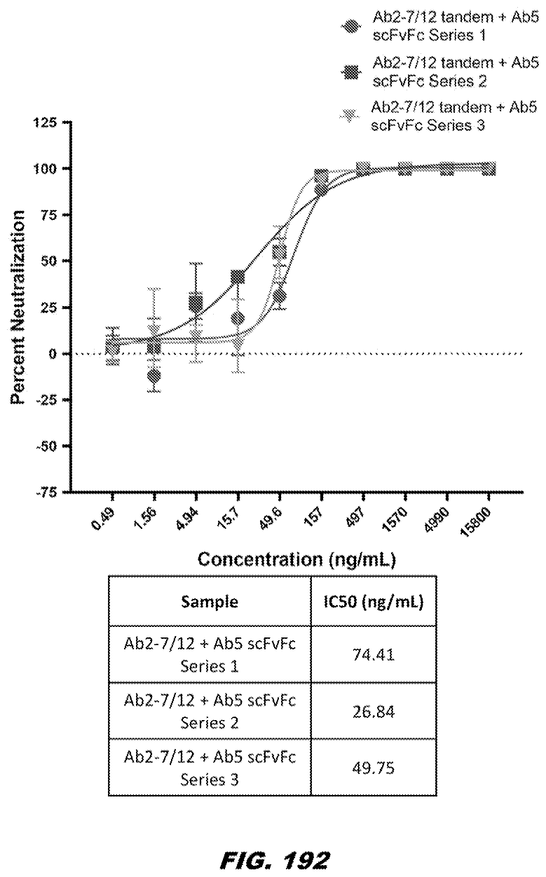

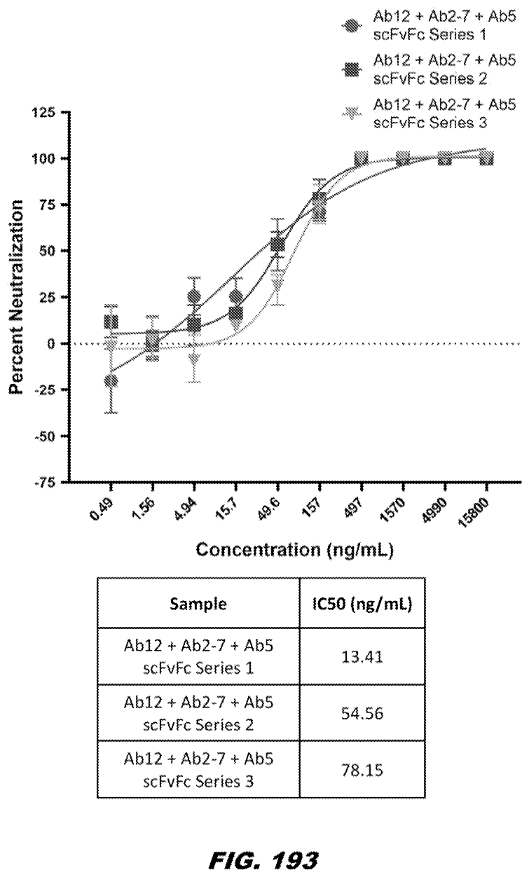

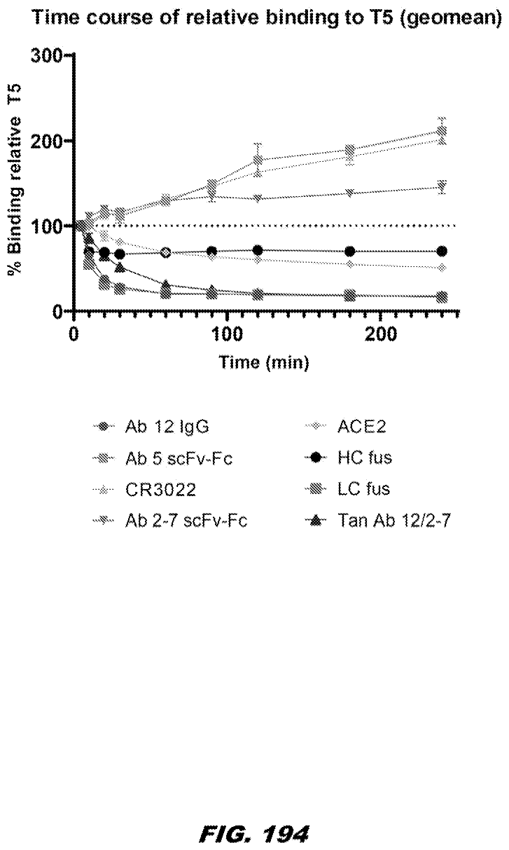

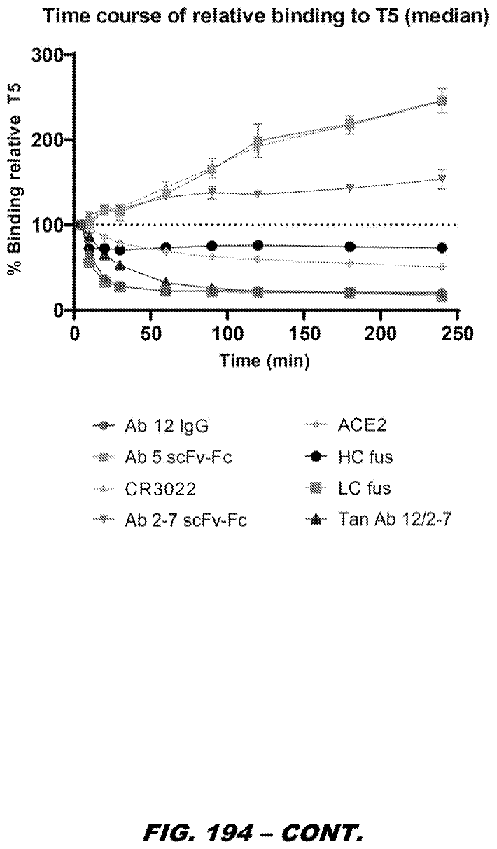

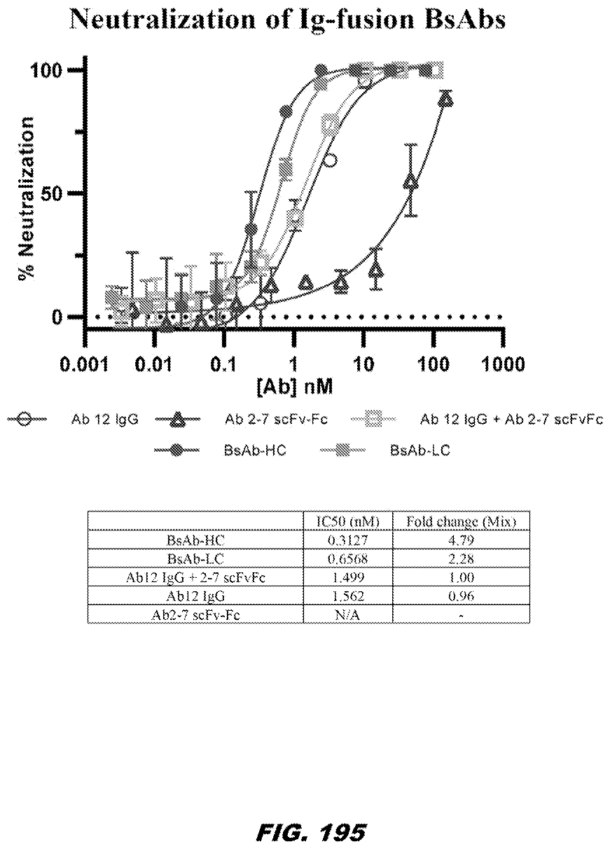

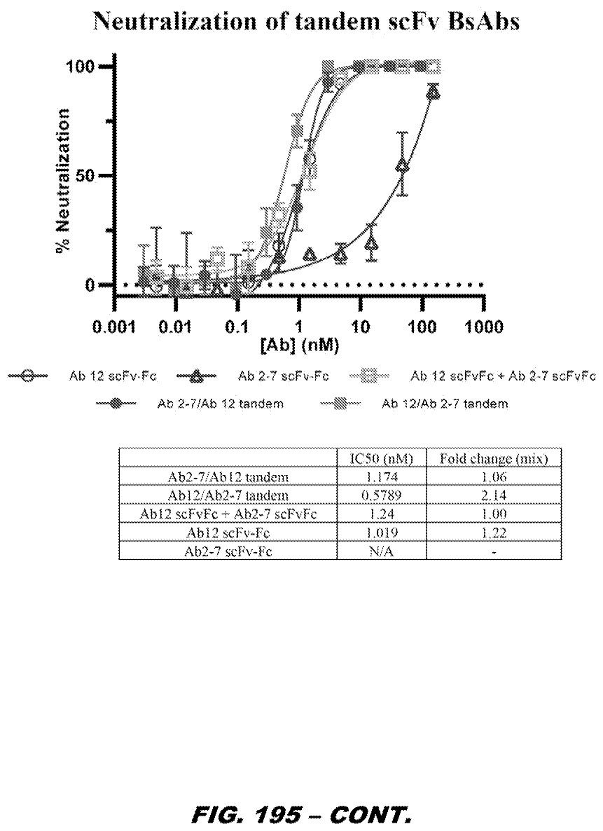

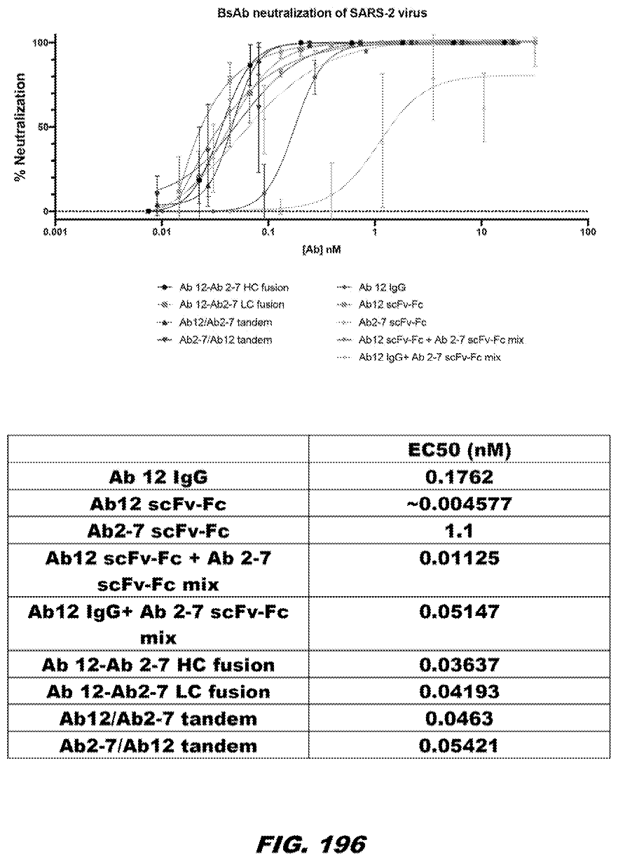

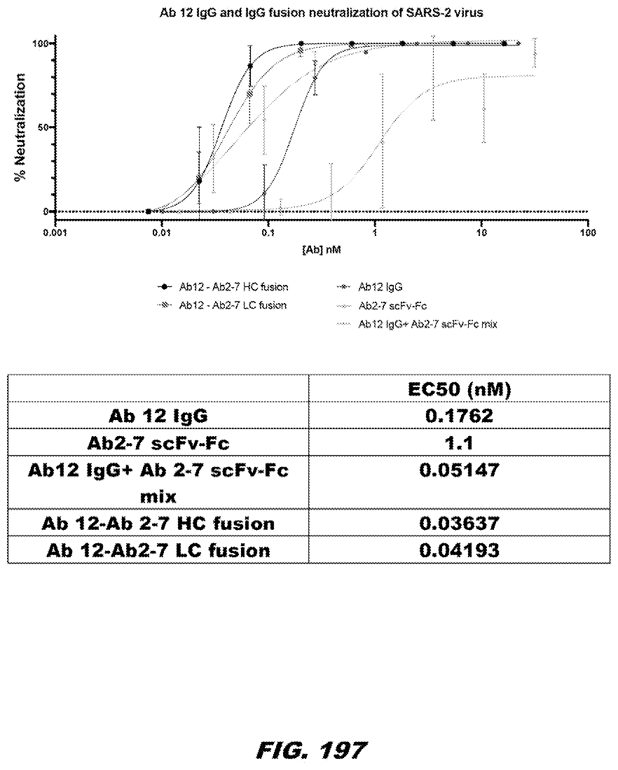

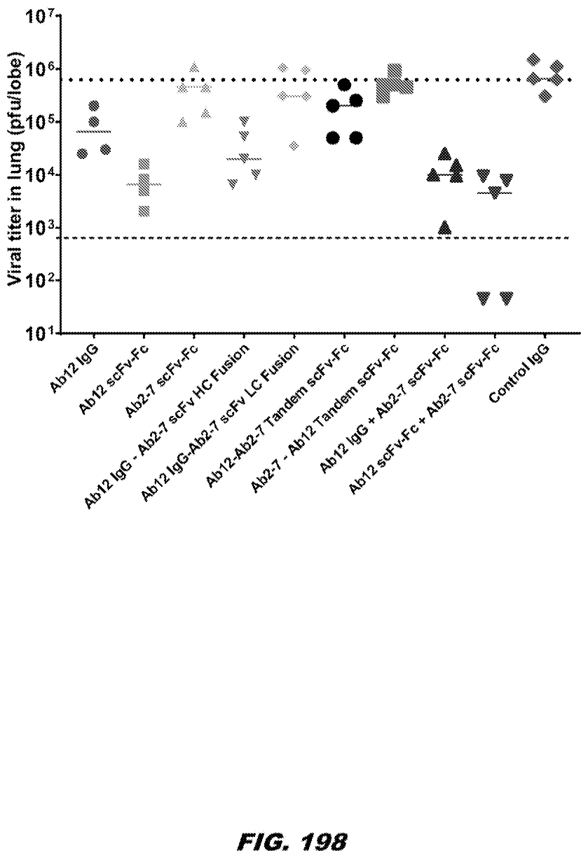

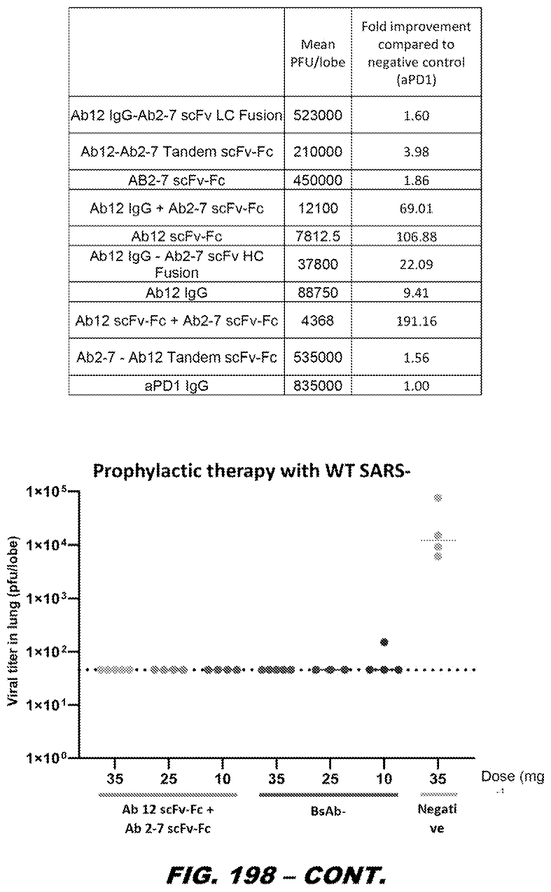

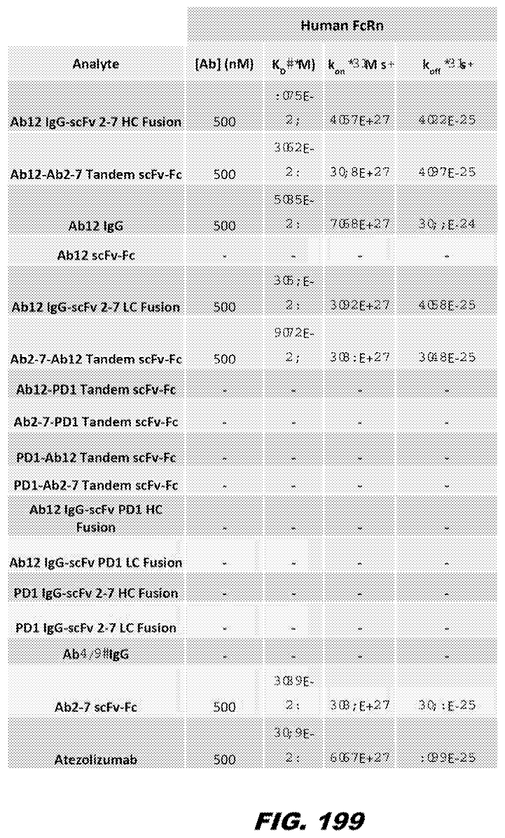

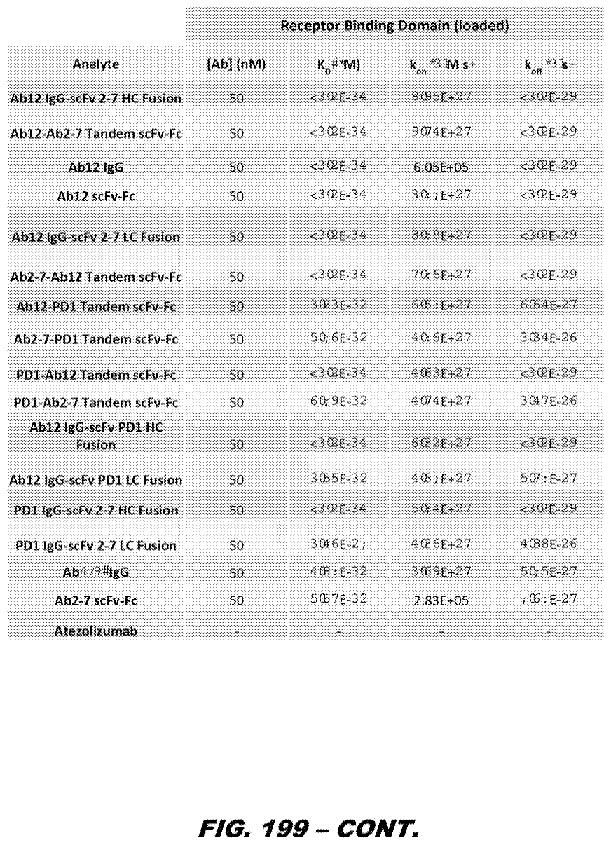

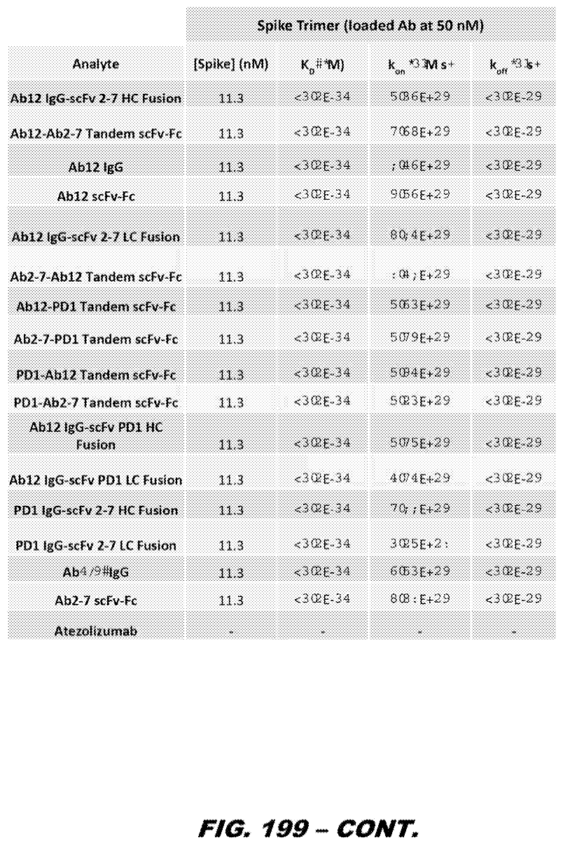

FIGS. 130 to 141 show the full sequences of the tandem and mono scFv arrangement, as well as the various Fc domains that were created. Mutation numbering in the KiH constructs uses IGMT numbering, see IMGT.org for conversion to Kabat or EU numbering. There are two hole constructs for each design, our current work focuses on the FLAG variant.

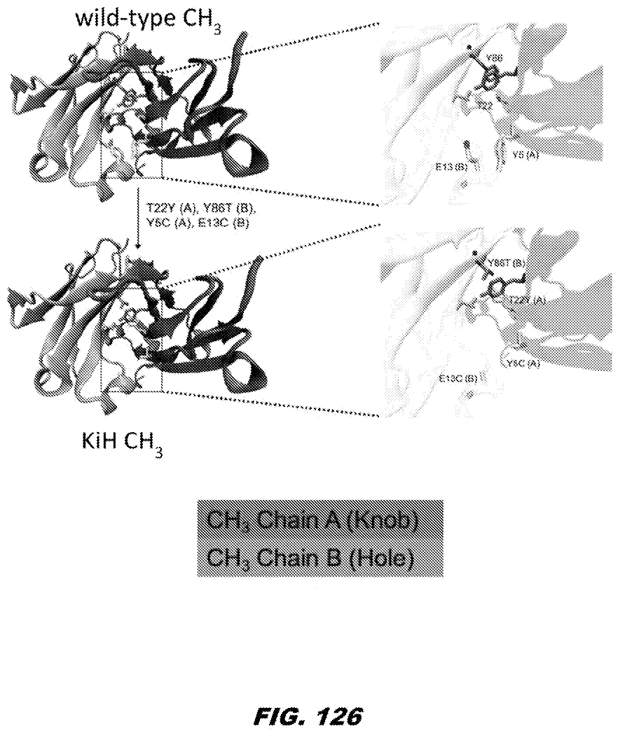

KiH Construct 1 (LT) Amino Acid Sequence (for yellow, red, and green residues see FIG. 126)—

CH3 Chain A-Mutated AA Sequence (SEQ ID NO: XX):
GQPREPQVCTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
CH3 Chain B-Mutated AA Sequence (SEQ ID NO: XX):
GQPREPQVYVYPPSRDCLTKNQVSLTCLVKGFYPSDIAVEWESNGQPEN
NYKTTPPVLDSDGSFFLTSKLTVDKSRWQQGNVFSCSVMHEALHNHYTQ
KSLSLSPGK

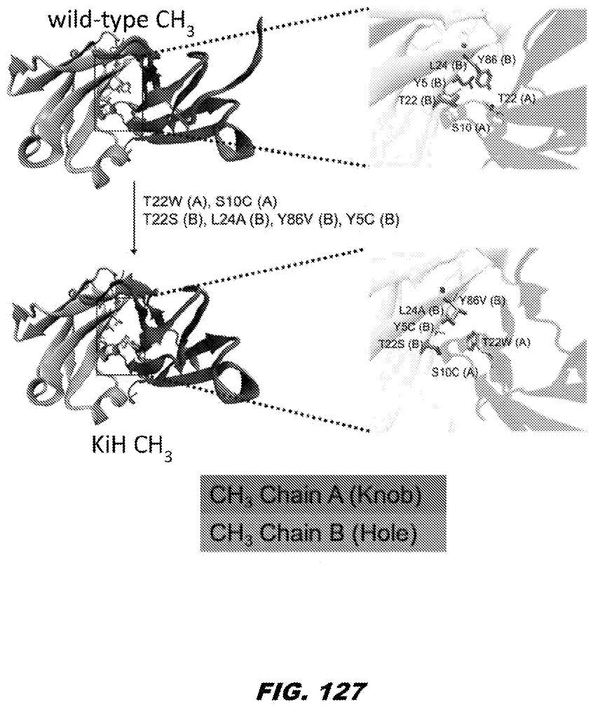

KiH Construct 2 (KiH_{S-S}) Amino Acid Sequence (for yellow, red, purple, and aqua residues see FIG. 127; see also Merchant et al., 1998 and Leaver-Fay et al., 2016)—

CH3 Chain A-Mutated AA Sequence (SEQ ID NO: XX):
GQPREPQVYTLPPCRDELTKNQVSLWCLVKGFYPSDIAVEWESNGQPEN
NYKTTPPVLDSDGSFFLYSKLTVDKSRWQQGNVFSCSVMHEALHNHYTQ
KSLSLSPGK
CH3 Chain B-Mutated AA Sequence (SEQ ID NO: XX):
GQPREPQVCTLPPSRDELTKNQVSLSCAVKGFYPSDIAVEWESNGQPEN
NYKTTPPVLDSDGSFFLVSKLTVDKSRWQQGNVFSCSVMHEALHNHYTQ
KSLSLSPGK

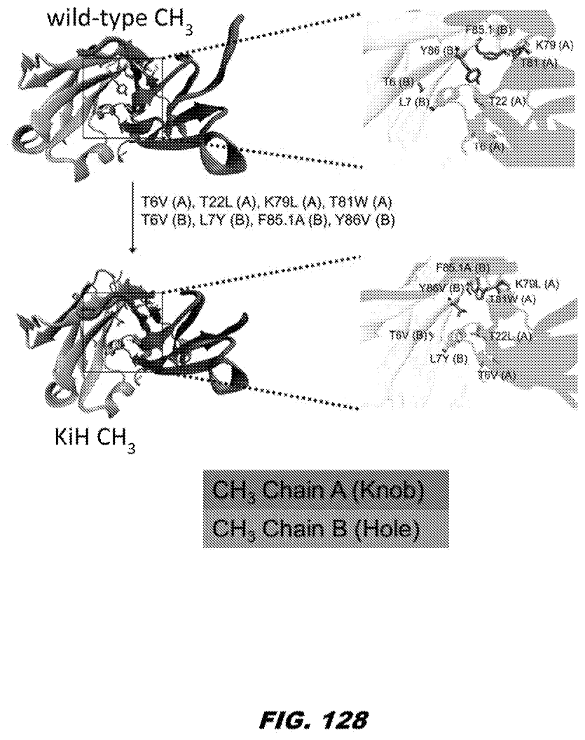

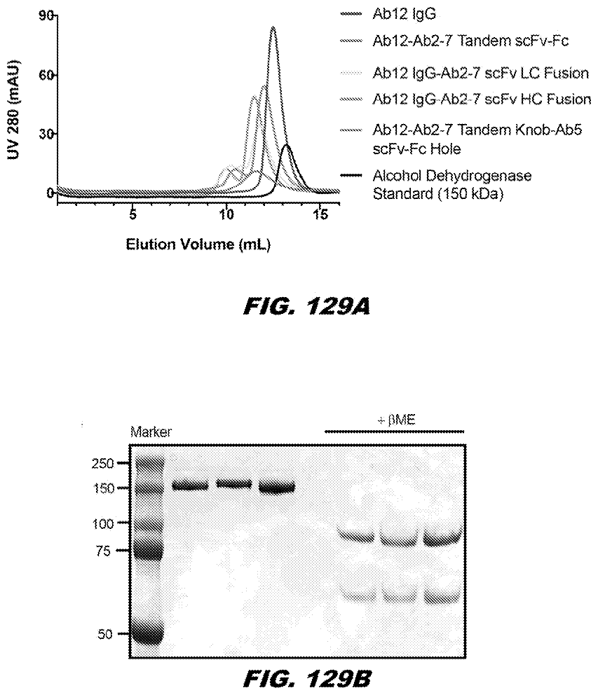

KiH Construct 3 (ZW1) Amino Acid Sequence (for yellow, red, purple, green, blue, pink, grey, and aqua residues see FIG. 128; see also Von Kreudenstein et al., 2013)

CH3 Chain A-Mutated AA Sequence (SEQ ID NO: XX):
GQPREPQVYVLPPSRDELTKNQVSLLCLVKGFYPSDIAVEWESNGQPEN
NYLTWPPVLDSDGSFFLYSKLTVDKSRWQQGNVFSCSVMHEALHNHYTQ
KSLSLSPGK
CH3 Chain B-Mutated AA Sequence (SEQ ID NO: XX):
GQPREPQVYVYPPSRDELTKNQVSLTCLVKGFYPSDIAVEWESNGQPEN
NYKTTPPVLDSDGSFALVSKLTVDKSRWQQGNVFSCSVMHEALHNHYTQ
KSLSLSPGK

### Example 10—Live Virus Neutralization Data

Monoclonal antibodies were diluted to 100 ug/ml by adding 50 μl of 1 mg/ml antibody to 450 μl Dulbecco's Phosphate Buffered Saline (DPBS)(Gibco™). A series of 10 half-log dilutions was then prepared in triplicate for each antibody in DPBS. Each dilution was incubated at 37° C. and 5% CO₂ for 1 hour with 1000 plaque forming units/ml (PFU/ml) of SARS-CoV-2 (isolate USA-WA1/2020), diluted in Dulbecco's Modified Eagle Medium (DMEM) (Gibco™) containing 2% fetal bovine serum (Gibco™) and antibiotic-antimycotic (Gibco™). Controls included DMEM containing 2% fetal bovine serum and antibiotic-antimycotic only as a negative control, 1000 PFU/ml SARS-CoV-2 incubated with DPBS, and 1000 PFU/ml SARS-CoV-2 incubated with DMEM. Two hundred microliters of each dilution or control were added to confluent monolayers of NR-596 Vero E6 cells in triplicate and incubated for 1 hour at 37° C. and 5% CO². The plates were gently rocked every 5-10 minutes to prevent monolayer drying. The monolayers were then overlaid with a 1:1 mixture of 2.5% Avicel® RC-591 microcrystalline cellulose and carboxymethylcellulose sodium (DuPont Nutrition & Biosciences, Wilmington, DE) and 2× Modified Eagle Medium (Temin's modification, Gibco™) supplemented with 2× antibiotic-antimycotic (Gibco™), 2× GlutaMAX (Gibco™) and 10% fetal bovine serum (Gibco™). Plates were incubated at 37° C. and 5% CO² for 2 days. The monolayers were fixed with 10% neutral buffered formalin and stained with 0.2% aqueous Gentian Violet (RICCA Chemicals, Arlington, TX) in 10% neutral buffered formalin for 30 min, followed by rinsing and plaque counting. The half maximal inhibitory concentrations (IC50) were calculated using GraphPad Prism 8.

### Example 11—Lung Lesion Analyses

Tissues originated from Syrian golden hamsters infected with SARS-CoV-2 and treated with monoclonal antibodies or left untreated. Photographs of whole plucks (lungs and heart) taken at necropsy were provided. One H&E stained slide was presented for each animal. Tissues examined were fixed in formalin for at least 96 hours prior to preparation. Embedding, slide preparation and staining were conducted per standard protocol. Only lung tissue was presented for examination. Lung consolidation percentages were determined as a function of the total observed area affected by consolidation, defined as collapsed alveoli, infiltration of mononuclear inflammatory cells, and darkened (plum colored) staining. Infiltrated foci are regions with significant numbers of infiltrating inflammatory mononuclear cells. These are often readily identifiable as blue/purple patches in the tissue section. Infiltrated airways were defined as large or small airways fully or partly (>10%) occluded by mononuclear inflammatory cells.

Gross and clinical pathology findings: Patchy consolidation was observed on the lungs, with some apparent improvement in treated animals. However, it was difficult to assess the degree of consolidation, as it was difficult to differentiate consolidation from blood on the surface of the organ in the photographs provided.

### Lung Lesion Score:

0: no lesions observed
1: 25% and under area of lesion coverage
2: 26%-49% area of lesion coverage
3: 50%-74% area of lesion coverage
4: 75% and above area of lesion coverage

TABLE II
Lung Lesion Scoring Table

| ID# | Gross lung lesion score | Full Lung lobe appearance |
|---|---|---|
| Control 1 | 2 | Yes, rounded edges of lobes |
| Control 2 | 1 | Yes, rounded edges of lobes |
| Control 3 | 1 | Yes, rounded edges of lobes |
| Control 4 | 0 | Yes, rounded edges of lobes |
| Control 5 | 2 | Yes, rounded edges of lobes |
| A12 | 2 | No rounding of love edges |
| A12 | 1 | No rounding of love edges |
| A12 | 1 | No rounding of love edges |
| A12 | 2 | No rounding of love edges |
| A12 | 1 | No rounding of love edges |

### Virus-Only:

General. Changes observed are consistent with viral interstitial pneumonia, namely alveolar wall thickening, alveolar collapse, and inflammatory cell infiltration. Airway obstruction by inflammatory cells common, and present, in all sections.

| | % Infiltrated | No. of | | |
|---|---|---|---|---|
| Animal | Airways | Infiltration Foci | Consolidation | Comments |
| 1 | 45% | 8 | 13% | None. |
| 2 | 60% | 7 | 29% | None. |
| 3 | 25% | 3 | 56% | None. |
| 4 | 40% | 4 | 50% | None. |
| 5 | 55% | 5 | 20% | None. |

Tissues from untreated animals displayed gross pathology and histopathology consistent with viral interstitial pneumonia. Inflammatory cell infiltration present in all sections, along with infiltration of large and small airways and perivascular cuffing. Severity of pathology was variable between animals.

Lung tissues from animals that received monoclonal antibodies displayed effectively identical pathology, though certain sections had notable infiltration of inflammatory cells into large airways. Pathology was moderately variable between animals.

Consolidation and airway infiltration were significantly improved in animals treated with AB12 (p<0.05, Mann-Whitney test). No improvement was found in the number of observed foci of inflammatory infiltration.

### Antibody 12:

General. Signs of typical histopathology associated with viral interstitial pneumonia (discussed previously) noted in all sections. Significantly improved consolidation relative to untreated animals. Some sections had notable infiltration of inflammatory cells into large airways.

| | % Infiltrated | No. of | | |
|---|---|---|---|---|
| Animal | Airways | Infiltration Foci | Consolidation | Comments |
| 1 | 10% | 1 | 0% | None. |
| 2 | 10% | 3 | 9% | None. |
| 3 | 30% | 4 | 8% | ** |
| 4 | 20% | 4 | 14% | None. |
| 5 | 40% | 5 | 3% | None. |

** Large airways with significant inflammatory cell infiltration noted

### Example 12—Syrian Golden Hamster Experiments

Syrian hamster SARS-CoV-2 virus challenge study. Animal challenge studies were conducted. 1 day before the challenge hamsters were microchipped. On day 0, hamsters were anesthetized with ketamine/xylazine and challenged with SARS-CoV-2 by the intranasal (IN) route with up to 10\hat{ }7 TCID50 (or 10\hat{ }6 PFU/ml) in a total volume up to 100 μL. The viral strain used is Wuhan Hu-1 strain, SARS-CoV strain 2019-nCoV/USA_WA1/2020 (WA1); GenBank: MN985325; GISAID: EPI_ISL_404895. For animal experiments passage 5 was used. The final challenge dose was 10000 plaque forming units diluted in sterile PBS. Body weight and body temperature were measured each day, starting at day 0. On day 1 post challenge (dpc) hamsters were treated with 5 mg/kg of monoclonal antibodies diluted in 0.5 ml of sterile PBS via intraperitoneal route (IP). The control group received an equal volume of sterile PBS via the same IP route. On day 3 post challenge the animals were sacrificed. At necropsy, terminal blood was collected into a labeled 3.5 mL SST vacutainer from the animals. Lungs were harvested for the groups.

Syrian golden hamster tissue processing and viral load determination. For the pathogenicity study, animals from each study group were euthanized on day 3 post challenge, and the lungs were harvested. Right lungs were placed in L15 medium supplemented with 10% fetal bovine serum (Gibco) and Antibiotic-Antimycotic solution (Gibco), flash-frozen in dry ice and stored at −80C until processing. Tissues were thawed and homogenized using the TissueLyser II system (Qiagen). Tissue homogenates were titrated on Vero E6 cell monolayers in 96-well plates to determine viral loads. 10× fold dilutions of the lung supernatants were incubated for 1 hour and replaced with 100 μLs of 0.9% methylcellulose in minimal essential medium (MEM) containing 10% fetal bovine serum (Quality Biologicals) and 0.1% gentamicin sulfate (Mediatech), followed by incubation at 37 C. Plates were fixed with 10% buffered formalin (Thermo Fisher) with subsequent removal from the biocontainment laboratories. Foci were visualized by staining monolayers with a mixture of 37 SARS-CoV-2 specific human antibodies kindly provided by Distributed Bio. As the secondary antibody, HRP-labeled goat anti-human IgG (SeraCare) was used at dilution 1:500. Primary and secondary antibodies were diluted in 1×DPBS with 5% milk. Plaques were revealed by AEC substrate (enQuire Bioreagents).

Syrian golden hamster histopathology. During necropsy, gross lesions were noted and representative lung tissues from the left lobe were collected in 10% formalin. After a 24-hour initial fixation at 4C, the lung tissues were transferred to fresh 10% formalin for an additional 48-hour fixation, prior to removal from containment. Formalin-fixed tissues were processed by standard histological procedures by the UTMB Anatomic Pathology Core. About 4 μm-thick sections were cut and stained with hematoxylin and eosin (HE). Sections of lungs were examined for the extent of inflammation, type of inflammatory foci, and changes in alveoli/alveolar septa/airways/blood vessels in parallel with sections from uninfected or control animals. The blinded tissue sections were semi-quantitatively scored for pathological lesions using the criteria described in Table III (51). Slides were scored by a trained staff member. Significance was assessed using a Kruskall-Wallis test with Dunn's post-hoc correction.

TABLE III
Lung Lesion Scores

| ID# | Gross lung lesion score | Full Lung lobe appearance |
|---|---|---|
| Control 1 | 2 | Yes, rounded edges of lobes |
| Control 2 | 1 | Yes, rounded edges of lobes |
| Control 3 | 1 | Yes, rounded edges of lobes |
| Control 4 | 0 | Yes, rounded edges of lobes |
| Control 5 | 2 | Yes, rounded edges of lobes |
| A12 | 2 | No rounding of love edges |
| A12 | 1 | No rounding of love edges |
| A12 | 1 | No rounding of love edges |
| A12 | 2 | No rounding of love edges |
| A12 | 1 | No rounding of love edges |

0: no lesions observed; 1: 25% and under area of lesion coverage; 2: 26%-49% area of lesion coverage; 3: 50%-74% area of lesion coverage; 4: 75% and above area of lesion coverage.

### Example 13—Spike Mutant Binding Studies

The table below shows examples of spike mutant binding to the SARS-COV-2 antibodies described herein.

| | | Loading | Loading | Loading | | | | |
|---|---|---|---|---|---|---|---|---|
| Sample | Dissoc. | Well | Sample | Conc. | | Conc. | | KD |
| ID | Loc. | Location | ID | (μg/ml) | Cycle | (nM) | Response | (M) |
| WT Spike | p1A12 | A1 | Ab 2 scFv- | 1 | 1 | 48.3 | 0.0652 | 1.99E−10 |
| | | | Fc | | | | | |
| WT Spike | p1A12 | A2 | Ab 5 scFv- | 1 | 2 | 48.3 | 0.0828 | 6.31E−12 |
| | | | Fc | | | | | |
| WT Spike | p1A12 | A3 | Ab 7 scFv- | 1 | 3 | 48.3 | 0.2424 | 7.00E−12 |
| | | | Fc | | | | | |
| WT Spike | p1A12 | A4 | Ab 8 scFv- | 1 | 4 | 48.3 | 0.118 | 7.15E−12 |
| | | | Fc | | | | | |
| D614G | p1B12 | B1 | Ab 2 scFv- | 1 | 1 | 48.3 | 0.1499 | 1.67E−10 |
| Spike | | | Fc | | | | | |
| D614G | p1B12 | B2 | Ab 5 scFv- | 1 | 2 | 48.3 | 0.1913 | 3.14E−12 |
| Spike | | | Fc | | | | | |
| D614G | p1B12 | B3 | Ab 7 scFv- | 1 | 3 | 48.3 | 0.345 | 2.74E−12 |
| Spike | | | Fc | | | | | |
| D614G | p1B12 | B4 | Ab 8 scFv- | 1 | 4 | 48.3 | 0.1871 | 7.30E−12 |
| Spike | | | Fc | | | | | |
| B.1.1.7 | p1C12 | C1 | Ab 2 scFv- | 1 | 1 | 48.4 | 0.1608 | 6.57E−10 |
| Spike | | | Fc | | | | | |
| B.1.1.7 | p1C12 | C2 | Ab 5 scFv- | 1 | 2 | 48.4 | 0.185 | 3.26E−10 |
| Spike | | | Fc | | | | | |
| B.1.1.7 | p1C12 | C3 | Ab 7 scFv- | 1 | 3 | 48.4 | 0.3565 | 1.10E−12 |
| Spike | | | Fc | | | | | |
| B.1.1.7 | p1C12 | C4 | Ab 8 scFv- | 1 | 4 | 48.4 | 0.2023 | 2.29E−12 |
| Spike | | | Fc | | | | | |
| B.1.351 | p1D12 | D1 | Ab 2 scFv- | 1 | 1 | 48.5 | 0.0439 | 5.03E−10 |
| Spike | | | Fc | | | | | |
| B.1.351 | p1D12 | D2 | Ab 5 scFv- | 1 | 2 | 48.5 | 0.1247 | 1.81E−11 |
| Spike | | | Fc | | | | | |
| B.1.351 | p1D12 | D3 | Ab 7 scFv- | 1 | 3 | 48.5 | 0.2329 | 1.67E−12 |
| Spike | | | Fc | | | | | |
| B.1.351 | p1D12 | D4 | Ab 8 scFv- | 1 | 4 | 48.5 | 0.0872 | 1.09E−09 |
| Spike | | | Fc | | | | | |
| P.1 Spike | p1F12 | F1 | Ab 2 scFv- | 1 | 1 | 48.3 | 0.0281 | 9.13E−12 |
| | | | Fc | | | | | |
| P.1 Spike | p1F12 | F2 | Ab 5 scFv- | 1 | 2 | 48.3 | 0.1146 | 7.71E−12 |
| | | | Fc | | | | | |
| P.1 Spike | p1F12 | F3 | Ab 7 scFv- | 1 | 3 | 48.3 | 0.242 | 1.09E−11 |
| | | | Fc | | | | | |
| P.1 Spike | p1F12 | F4 | Ab 8 scFv- | 1 | 4 | 48.3 | 0.0784 | 1.06E−11 |
| | | | Fc | | | | | |
| WT Spike | p1A12 | A5 | Ab 2-2 | 1 | 1 | 48.3 | 0.0747 | 9.01E−12 |
| | | | scFv-Fc | | | | | |
| WT Spike | p1A12 | A6 | Ab 2-7 | 1 | 2 | 48.3 | 0.0672 | 7.85E−12 |
| | | | scFv-Fc | | | | | |
| WT Spike | p1A12 | A7 | Ab 2-10 | 1 | 3 | 48.3 | 0.0423 | 5.65E−12 |
| | | | scFv-Fc | | | | | |
| WT Spike | p1A12 | A8 | Ab 12 scFv- | 1 | 4 | 48.3 | 0.1857 | 8.31E−12 |
| | | | Fc | | | | | |
| WT Spike | p1A12 | A9 | Ab 13 scFv- | 1 | 5 | 48.3 | 0.0732 | 8.55E−12 |
| | | | Fc | | | | | |
| D614G | p1B12 | B5 | Ab 2-2 | 1 | 1 | 48.3 | 0.1324 | 8.91E−12 |
| Spike | | | scFv-Fc | | | | | |
| D614G | p1B12 | B6 | Ab 2-7 | 1 | 2 | 48.3 | 0.1161 | 9.93E−12 |
| Spike | | | scFv-Fc | | | | | |
| D614G | p1B12 | B7 | Ab 2-10 | 1 | 3 | 48.3 | 0.0723 | 8.87E−12 |
| Spike | | | scFv-Fc | | | | | |
| D614G | p1B12 | B8 | Ab 12 scFv- | 1 | 4 | 48.3 | 0.2182 | 1.12E−12 |
| Spike | | | Fc | | | | | |
| D614G | p1B12 | B9 | Ab 13 scFv- | 1 | 5 | 48.3 | 0.1071 | 8.10E−12 |
| Spike | | | Fc | | | | | |
| B.1.1.7 | p1C12 | C5 | Ab 2-2 | 1 | 1 | 48.4 | 0.1348 | 3.06E−12 |
| Spike | | | scFv-Fc | | | | | |
| B.1.1.7 | p1C12 | C6 | Ab 2-7 | 1 | 2 | 48.4 | 0.1272 | 3.79E−10 |
| Spike | | | scFv-Fc | | | | | |
| B.1.1.7 | p1C12 | C7 | Ab 2-10 | 1 | 3 | 48.4 | 0.0939 | 6.82E−10 |
| Spike | | | scFv-Fc | | | | | |
| B.1.1.7 | p1C12 | C8 | Ab 12 scFv- | 1 | 4 | 48.4 | 0.1773 | 2.73E−12 |
| Spike | | | Fc | | | | | |
| B.1.1.7 | p1C12 | C9 | Ab 13 scFv- | 1 | 5 | 48.4 | 0.1134 | 1.91E−12 |
| Spike | | | Fc | | | | | |
| B.1.351 | p1D12 | D5 | Ab 2-2 | 1 | 1 | 48.5 | 0.1185 | 6.53E−12 |
| Spike | | | scFv-Fc | | | | | |
| B.1.351 | p1D12 | D6 | Ab 2-7 | 1 | 2 | 48.5 | 0.1298 | 3.60E−10 |
| Spike | | | scFv-Fc | | | | | |
| B.1.351 | p1D12 | D7 | Ab 2-10 | 1 | 3 | 48.5 | 0.1032 | 5.23E−10 |
| Spike | | | scFv-Fc | | | | | |
| B.1.351 | p1D12 | D8 | Ab 12 scFv- | 1 | 4 | 48.5 | 0.0737 | 1.45E−09 |
| Spike | | | Fc | | | | | |
| B.1.351 | p1D12 | D9 | Ab 13 scFv- | 1 | 5 | 48.5 | 0.1027 | 7.35E−10 |
| Spike | | | Fc | | | | | |
| P.1 Spike | p1F12 | F5 | Ab 2-2 | 1 | 1 | 48.3 | 0.1434 | 8.94E−12 |
| | | | scFv-Fc | | | | | |
| P.1 Spike | p1F12 | F6 | Ab 2-7 | 1 | 2 | 48.3 | 0.152 | 7.04E−12 |
| | | | scFv-Fc | | | | | |
| P.1 Spike | p1F12 | F7 | Ab 2-10 | 1 | 3 | 48.3 | 0.0969 | 6.67E−12 |
| | | | scFv-Fc | | | | | |
| P.1 Spike | p1F12 | F8 | Ab 12 scFv- | 1 | 4 | 48.3 | 0.0718 | 8.55E−12 |
| | | | Fc | | | | | |
| P.1 Spike | p1F12 | F9 | Ab 13 scFv- | 1 | 5 | 48.3 | 0.1122 | 4.88E−12 |
| | | | Fc | | | | | |
| WT Spike | p1A12 | A1 | Ab 14 scFv- | 1 | 1 | 48.3 | 0.3126 | 8.22E−12 |
| | | | Fc | | | | | |
| WT Spike | p1A12 | A2 | Ab 16 scFv- | 1 | 2 | 48.3 | 0.346 | 1.18E−11 |
| | | | Fc | | | | | |
| WT Spike | p1A12 | A3 | Ab 17 scFv- | 1 | 3 | 48.3 | 0.2478 | 6.35E−12 |
| | | | Fc | | | | | |
| WT Spike | p1A12 | A4 | Ab 18 scFv- | 1 | 4 | 48.3 | 0.2165 | 9.07E−12 |
| | | | Fc | | | | | |
| D614G | p1B12 | B1 | Ab 14 scFv- | 1 | 1 | 48.3 | 0.4015 | 8.12E−12 |
| Spike | | | Fc | | | | | |
| D614G | p1B12 | B2 | Ab 16 scFv- | 1 | 2 | 48.3 | 0.434 | 8.35E−12 |
| Spike | | | Fc | | | | | |
| D614G | p1B12 | B3 | Ab 17 scFv- | 1 | 3 | 48.3 | 0.3573 | 6.80E−12 |
| Spike | | | Fc | | | | | |
| D614G | p1B12 | B4 | Ab 18 scFv- | 1 | 4 | 48.3 | 0.2909 | 6.33E−12 |
| Spike | | | Fc | | | | | |
| B.1.1.7 | p1C12 | C1 | Ab 14 scFv- | 1 | 1 | 48.4 | 0.355 | 7.98E−12 |
| Spike | | | Fc | | | | | |
| B.1.1.7 | p1C12 | C2 | Ab 16 scFv- | 1 | 2 | 48.4 | 0.3876 | 2.36E−12 |
| Spike | | | Fc | | | | | |
| B.1.1.7 | p1C12 | C3 | Ab 17 scFv- | 1 | 3 | 48.4 | 0.3312 | 1.85E−12 |
| Spike | | | Fc | | | | | |
| B.1.1.7 | p1C12 | C4 | Ab 18 scFv- | 1 | 4 | 48.4 | 0.2559 | 9.28E−12 |
| Spike | | | Fc | | | | | |
| B.1.351 | p1D12 | D1 | Ab 14 scFv- | 1 | 1 | 48.5 | 0.2534 | 3.10E−12 |
| Spike | | | Fc | | | | | |
| B.1.351 | p1D12 | D2 | Ab 16 scFv- | 1 | 2 | 48.5 | 0.4045 | <1.0E−12 |
| Spike | | | Fc | | | | | |
| B.1.351 | p1D12 | D3 | Ab 17 scFv- | 1 | 3 | 48.5 | 0.1994 | 5.62E−10 |
| Spike | | | Fc | | | | | |
| B.1.351 | p1D12 | D4 | Ab 18 scFv- | 1 | 4 | 48.5 | 0.1204 | 1.34E−09 |
| Spike | | | Fc | | | | | |
| P.1 Spike | p1F12 | F1 | Ab 14 scFv- | 1 | 1 | 48.3 | 0.2699 | 9.75E−12 |
| | | | Fc | | | | | |
| P.1 Spike | p1F12 | F2 | Ab 16 scFv- | 1 | 2 | 48.3 | 0.3497 | 7.93E−12 |
| | | | Fc | | | | | |
| P.1 Spike | p1F12 | F3 | Ab 17 scFv- | 1 | 3 | 48.3 | 0.1772 | 8.00E−12 |
| | | | Fc | | | | | |
| P.1 Spike | p1F12 | F4 | Ab 18 scFv- | 1 | 4 | 48.3 | 0.1088 | 2.97E−12 |
| | | | Fc | | | | | |
| WT Spike | p1A12 | A5 | Ab 19 scFv- | 1 | 1 | 48.3 | 0.1214 | 8.90E−12 |
| | | | Fc | | | | | |
| WT Spike | p1A12 | A6 | Ab 20 scFv- | 1 | 2 | 48.3 | 0.0985 | 9.01E−12 |
| | | | Fc | | | | | |
| WT Spike | p1A12 | A7 | Ab 21 scFv- | 1 | 3 | 48.3 | 0.1473 | 9.25E−12 |
| | | | Fc | | | | | |
| WT Spike | p1A12 | A8 | Ab 22 scFv- | 1 | 4 | 48.3 | 0.1001 | 8.87E−12 |
| | | | Fc | | | | | |
| WT Spike | p1A12 | A9 | Ab 23 scFv- | 1 | 5 | 48.3 | 0.0845 | 8.84E−12 |
| | | | Fc | | | | | |
| D614G | p1B12 | B5 | Ab 19 scFv- | 1 | 1 | 48.3 | 0.2371 | 1.09E−11 |
| Spike | | | Fc | | | | | |
| D614G | p1B12 | B6 | Ab 20 scFv- | 1 | 2 | 48.3 | 0.1899 | 8.64E−12 |
| Spike | | | Fc | | | | | |
| D614G | p1B12 | B7 | Ab 21 scFv- | 1 | 3 | 48.3 | 0.2275 | 8.00E−12 |
| Spike | | | Fc | | | | | |
| D614G | p1B12 | B8 | Ab 22 scFv- | 1 | 4 | 48.3 | 0.1713 | 8.89E−12 |
| Spike | | | Fc | | | | | |
| D614G | p1B12 | B9 | Ab 23 scFv- | 1 | 5 | 48.3 | 0.1623 | 8.82E−12 |
| Spike | | | Fc | | | | | |
| B.1.1.7 | p1C12 | C5 | Ab 19 scFv- | 1 | 1 | 48.4 | 0.2032 | 1.03E−11 |
| Spike | | | Fc | | | | | |
| B.1.1.7 | p1C12 | C6 | Ab 20 scFv- | 1 | 2 | 48.4 | 0.1733 | 2.60E−12 |
| Spike | | | Fc | | | | | |
| B.1.1.7 | p1C12 | C7 | Ab 21 scFv- | 1 | 3 | 48.4 | 0.1989 | 1.47E−12 |
| Spike | | | Fc | | | | | |
| B.1.1.7 | p1C12 | C8 | Ab 22 scFv- | 1 | 4 | 48.4 | 0.1485 | 9.13E−12 |
| Spike | | | Fc | | | | | |
| B.1.1.7 | p1C12 | C9 | Ab 23 scFv- | 1 | 5 | 48.4 | 0.1348 | 9.11E−12 |
| Spike | | | Fc | | | | | |
| B.1.351 | p1D12 | D5 | Ab 19 scFv- | 1 | 1 | 48.5 | 0.2584 | 1.18E−11 |
| Spike | | | Fc | | | | | |
| B.1.351 | p1D12 | D6 | Ab 20 scFv- | 1 | 2 | 48.5 | 0.2131 | 7.03E−12 |
| Spike | | | Fc | | | | | |
| B.1.351 | p1D12 | D7 | Ab 21 scFv- | 1 | 3 | 48.5 | 0.1483 | 3.69E−10 |
| Spike | | | Fc | | | | | |
| B.1.351 | p1D12 | D8 | Ab 22 scFv- | 1 | 4 | 48.5 | 0.1833 | 3.18E−12 |
| Spike | | | Fc | | | | | |
| B.1.351 | p1D12 | D9 | Ab 23 scFv- | 1 | 5 | 48.5 | 0.1892 | 7.58E−12 |
| Spike | | | Fc | | | | | |
| P.1 Spike | p1F12 | F5 | Ab 19 scFv- | 1 | 1 | 48.3 | 0.2044 | 8.94E−12 |
| | | | Fc | | | | | |
| P.1 Spike | p1F12 | F6 | Ab 20 scFv- | 1 | 2 | 48.3 | 0.1695 | 1.12E−11 |
| | | | Fc | | | | | |
| P.1 Spike | p1F12 | F7 | Ab 21 scFv- | 1 | 3 | 48.3 | 0.1031 | 9.52E−12 |
| | | | Fc | | | | | |
| P.1 Spike | p1F12 | F8 | Ab 22 scFv- | 1 | 4 | 48.3 | 0.1363 | 8.85E−12 |
| | | | Fc | | | | | |
| P.1 Spike | p1F12 | F9 | Ab 23 scFv- | 1 | 5 | 48.3 | 0.1228 | 8.79E−12 |
| | | | Fc | | | | | |
| WT Spike | p1A12 | A1 | Ab 25 scFv- | 1 | 1 | 48.3 | 0.0219 | 8.90E−12 |
| | | | Fc | | | | | |
| WT Spike | p1A12 | A2 | Ab 26 scFv- | 1 | 2 | 48.3 | 0.1853 | 1.10E−11 |
| | | | Fc | | | | | |
| WT Spike | p1A12 | A3 | Ab 27 scFv- | 1 | 3 | 48.3 | 0.2015 | 8.78E−12 |
| | | | Fc | | | | | |
| WT Spike | p1A12 | A4 | Ab 28 scFv- | 1 | 4 | 48.3 | 0.1318 | 8.90E−12 |
| | | | Fc | | | | | |
| D614G | p1B12 | B1 | Ab 25 scFv- | 1 | 1 | 48.3 | 0.0425 | 8.56E−12 |
| Spike | | | Fc | | | | | |
| D614G | p1B12 | B2 | Ab 26 scFv- | 1 | 2 | 48.3 | 0.3036 | 9.97E−12 |
| Spike | | | Fc | | | | | |
| D614G | p1B12 | B3 | Ab 27 scFv- | 1 | 3 | 48.3 | 0.2756 | 7.53E−12 |
| Spike | | | Fc | | | | | |
| D614G | p1B12 | B4 | Ab 28 scFv- | 1 | 4 | 48.3 | 0.1986 | 9.41E−12 |
| Spike | | | Fc | | | | | |
| B.1.1.7 | p1C12 | C1 | Ab 25 scFv- | 1 | 1 | 48.4 | 0.0668 | 4.27E−10 |
| Spike | | | Fc | | | | | |
| B.1.1.7 | p1C12 | C2 | Ab 26 scFv- | 1 | 2 | 48.4 | 0.3357 | 1.37E−12 |
| Spike | | | Fc | | | | | |
| B.1.1.7 | p1C12 | C3 | Ab 27 scFv- | 1 | 3 | 48.4 | 0.2805 | 1.57E−12 |
| Spike | | | Fc | | | | | |
| B.1.1.7 | p1C12 | C4 | Ab 28 scFv- | 1 | 4 | 48.4 | 0.2057 | 1.49E−12 |
| Spike | | | Fc | | | | | |
| B.1.351 | p1D12 | D1 | Ab 25 scFv- | 1 | 1 | 48.5 | 0.0577 | 1.13E−12 |
| Spike | | | Fc | | | | | |
| B.1.351 | p1D12 | D2 | Ab 26 scFv- | 1 | 2 | 48.5 | 0.2143 | 1.78E−12 |
| Spike | | | Fc | | | | | |
| B.1.351 | p1D12 | D3 | Ab 27 scFv- | 1 | 3 | 48.5 | 0.0974 | 1.25E−12 |
| Spike | | | Fc | | | | | |
| B.1.351 | p1D12 | D4 | Ab 28 scFv- | 1 | 4 | 48.5 | 0.1197 | 6.38E−12 |
| Spike | | | Fc | | | | | |
| P.1 Spike | p1F12 | F1 | Ab 25 scFv- | 1 | 1 | 48.3 | 0.0396 | 8.72E−12 |
| | | | Fc | | | | | |
| P.1 Spike | p1F12 | F2 | Ab 26 scFv- | 1 | 2 | 48.3 | 0.1877 | 1.03E−11 |
| | | | Fc | | | | | |
| P.1 Spike | p1F12 | F3 | Ab 27 scFv- | 1 | 3 | 48.3 | 0.0795 | 8.90E−12 |
| | | | Fc | | | | | |
| P.1 Spike | p1F12 | F4 | Ab 28 scFv- | 1 | 4 | 48.3 | 0.1023 | 8.91E−12 |
| | | | Fc | | | | | |
| WT Spike | p1A12 | A5 | Ab 29 scFv- | 1 | 1 | 48.3 | 0.126 | 1.49E−10 |
| | | | Fc | | | | | |
| WT Spike | p1A12 | A6 | Ab 30 scFv- | 1 | 2 | 48.3 | 0.1075 | 1.02E−12 |
| | | | Fc | | | | | |
| WT Spike | p1A12 | A7 | Ab 31 scFv- | 1 | 3 | 48.3 | 0.1009 | <1.0E−12 |
| | | | Fc | | | | | |
| WT Spike | p1A12 | A8 | Ab 32 scFv- | 1 | 4 | 48.3 | 0.1305 | 6.32E−12 |
| | | | Fc | | | | | |
| WT Spike | p1A12 | A9 | Ab 34 scFv- | 1 | 5 | 48.3 | 0.1002 | 6.58E−12 |
| | | | Fc | | | | | |
| D614G | p1B12 | B5 | Ab 29 scFv- | 1 | 1 | 48.3 | 0.2015 | 1.04E−12 |
| Spike | | | Fc | | | | | |
| D614G | p1B12 | B6 | Ab 30 scFv- | 1 | 2 | 48.3 | 0.1789 | 7.43E−12 |
| Spike | | | Fc | | | | | |
| D614G | p1B12 | B7 | Ab 31 scFv- | 1 | 3 | 48.3 | 0.1679 | 9.03E−12 |
| Spike | | | Fc | | | | | |
| D614G | p1B12 | B8 | Ab 32 scFv- | 1 | 4 | 48.3 | 0.2042 | 7.26E−12 |
| Spike | | | Fc | | | | | |
| D614G | p1B12 | B9 | Ab 34 scFv- | 1 | 5 | 48.3 | 0.19 | 9.15E−12 |
| Spike | | | Fc | | | | | |
| B.1.1.7 | p1C12 | C5 | Ab 29 scFv- | 1 | 1 | 48.4 | 0.1185 | 6.93E−10 |
| Spike | | | Fc | | | | | |
| B.1.1.7 | p1C12 | C6 | Ab 30 scFv- | 1 | 2 | 48.4 | 0.1389 | <1.0E−12 |
| Spike | | | Fc | | | | | |
| B.1.1.7 | p1C12 | C7 | Ab 31 scFv- | 1 | 3 | 48.4 | 0.1345 | <1.0E−12 |
| Spike | | | Fc | | | | | |
| B.1.1.7 | p1C12 | C8 | Ab 32 scFv- | 1 | 4 | 48.4 | 0.1506 | 2.02E−12 |
| Spike | | | Fc | | | | | |
| B.1.1.7 | p1C12 | C9 | Ab 34 scFv- | 1 | 5 | 48.4 | 0.1487 | 3.71E−12 |
| Spike | | | Fc | | | | | |
| B.1.351 | p1D12 | D5 | Ab 29 scFv- | 1 | 1 | 48.5 | 0.0418 | 3.71E−10 |
| Spike | | | Fc | | | | | |
| B.1.351 | p1D12 | D6 | Ab 30 scFv- | 1 | 2 | 48.5 | 0.0883 | <1.0E−12 |
| Spike | | | Fc | | | | | |
| B.1.351 | p1D12 | D7 | Ab 31 scFv- | 1 | 3 | 48.5 | 0.09 | <1.0E−12 |
| Spike | | | Fc | | | | | |
| B.1.351 | p1D12 | D8 | Ab 32 scFv- | 1 | 4 | 48.5 | 0.1152 | 7.11E−12 |
| Spike | | | Fc | | | | | |
| B.1.351 | p1D12 | D9 | Ab 34 scFv- | 1 | 5 | 48.5 | 0.1062 | 2.69E−12 |
| Spike | | | Fc | | | | | |
| P.1 Spike | p1F12 | F5 | Ab 29 scFv- | 1 | 1 | 48.3 | 0.0232 | 8.45E−12 |
| | | | Fc | | | | | |
| P.1 Spike | p1F12 | F6 | Ab 30 scFv- | 1 | 2 | 48.3 | 0.0766 | 9.02E−12 |
| | | | Fc | | | | | |
| P.1 Spike | p1F12 | F7 | Ab 31 scFv- | 1 | 3 | 48.3 | 0.0789 | 8.97E−12 |
| | | | Fc | | | | | |
| P.1 Spike | p1F12 | F8 | Ab 32 scFv- | 1 | 4 | 48.3 | 0.1032 | 8.87E−12 |
| | | | Fc | | | | | |
| P.1 Spike | p1F12 | F9 | Ab 34 scFv- | 1 | 5 | 48.3 | 0.0973 | 8.85E−12 |
| | | | Fc | | | | | |
| WT Spike | p1A12 | A1 | Ab 35 scFv- | 1 | 1 | 48.3 | 0.1085 | 8.90E−12 |
| | | | Fc | | | | | |
| WT Spike | p1A12 | A2 | Ab 36 scFv- | 1 | 2 | 48.3 | 0.076 | 9.01E−12 |
| | | | Fc | | | | | |
| WT Spike | p1A12 | A3 | Ab 37 scFv- | 1 | 3 | 48.3 | 0.0801 | 9.00E−12 |
| | | | Fc | | | | | |
| WT Spike | p1A12 | A4 | Ab 38 scFv- | 1 | 4 | 48.3 | 0.2736 | 7.86E−12 |
| | | | Fc | | | | | |
| WT Spike | p1A12 | A5 | Ab 39 scFv- | 1 | 5 | 48.3 | 0.3695 | 2.04E−12 |
| | | | Fc | | | | | |
| D614G | p1B12 | B1 | Ab 35 scFv- | 1 | 1 | 48.3 | 0.2332 | 8.69E−12 |
| Spike | | | Fc | | | | | |
| D614G | p1B12 | B2 | Ab 36 scFv- | 1 | 2 | 48.3 | 0.1619 | 7.42E−12 |
| Spike | | | Fc | | | | | |
| D614G | p1B12 | B3 | Ab 37 scFv- | 1 | 3 | 48.3 | 0.1568 | 8.54E−12 |
| Spike | | | Fc | | | | | |
| D614G | p1B12 | B4 | Ab 38 scFv- | 1 | 4 | 48.3 | 0.3275 | 3.37E−12 |
| Spike | | | Fc | | | | | |
| D614G | p1B12 | B5 | Ab 39 scFv- | 1 | 5 | 48.3 | 0.4581 | <1.0E−12 |
| Spike | | | Fc | | | | | |
| B.1.1.7 | p1C12 | C1 | Ab 35 scFv- | 1 | 1 | 48.4 | 0.1902 | 7.96E−12 |
| Spike | | | Fc | | | | | |
| B.1.1.7 | p1C12 | C2 | Ab 36 scFv- | 1 | 2 | 48.4 | 0.1462 | 5.16E−12 |
| Spike | | | Fc | | | | | |
| B.1.1.7 | p1C12 | C3 | Ab 37 scFv- | 1 | 3 | 48.4 | 0.1477 | 1.28E−12 |
| Spike | | | Fc | | | | | |
| B.1.1.7 | p1C12 | C4 | Ab 38 scFv- | 1 | 4 | 48.4 | 0.2667 | 1.47E−12 |
| Spike | | | Fc | | | | | |
| B.1.1.7 | p1C12 | C5 | Ab 39 scFv- | 1 | 5 | 48.4 | 0.2933 | <1.0E−12 |
| Spike | | | Fc | | | | | |
| B.1.351 | p1D12 | D1 | Ab 35 scFv- | 1 | 1 | 48.5 | 0.165 | 9.16E−12 |
| Spike | | | Fc | | | | | |
| B.1.351 | p1D12 | D2 | Ab 36 scFv- | 1 | 2 | 48.5 | 0.1342 | 8.07E−12 |
| Spike | | | Fc | | | | | |
| B.1.351 | p1D12 | D3 | Ab 37 scFv- | 1 | 3 | 48.5 | 0.1182 | 6.69E−12 |
| Spike | | | Fc | | | | | |
| B.1.351 | p1D12 | D4 | Ab 38 scFv- | 1 | 4 | 48.5 | 0.2343 | 7.62E−12 |
| Spike | | | Fc | | | | | |
| B.1.351 | p1D12 | D5 | Ab 39 scFv- | 1 | 5 | 48.5 | 0.1666 | 1.83E−12 |
| Spike | | | Fc | | | | | |
| P.1 Spike | p1F12 | F1 | Ab 35 scFv- | 1 | 1 | 48.3 | 0.1546 | 8.90E−12 |
| | | | Fc | | | | | |
| P.1 Spike | p1F12 | F2 | Ab 36 scFv- | 1 | 2 | 48.3 | 0.1128 | 8.89E−12 |
| | | | Fc | | | | | |
| P.1 Spike | p1F12 | F3 | Ab 37 scFv- | 1 | 3 | 48.3 | 0.1083 | 8.99E−12 |
| | | | Fc | | | | | |
| P.1 Spike | p1F12 | F4 | Ab 38 scFv- | 1 | 4 | 48.3 | 0.2283 | 7.23E−12 |
| | | | Fc | | | | | |
| P.1 Spike | p1F12 | F5 | Ab 39 scFv- | 1 | 5 | 48.3 | 0.1577 | 1.12E−11 |
| | | | Fc | | | | | |

### Example 14—Spike Shedding Experiments

2E5 293T cells stably expressing SARS-2 spike were harvested and resuspended in 50 ul FACS buffer+20 uM cycloheximide in each well of a 96 well V bottom plate. Antibodies were diluted to 200 nM in FACS buffer+20 uM cycloheximide and aliquoted into a deep well 96 well plate. Ab and cell plates were incubated at 37° C. for 15 min to allow for equilibration. At each time point, 50 ul of ab mix was added to the cells for a final ab concentration of 100 nM in each well. Plate was incubated at 37° C. through out the experiment. After the final time point, the reaction was quenched with the addition of 150 ul cold FACS buffer followed by centrifugation in a pre-chilled centrifuge (4° C.) at 750 g for 5 min. Cells and wash buffers were maintained at 4° C. for the remainder of the experiment. Cells were stained with in 100 ul FACS buffer with 1 ul anti-hFc-APC (Biolegend 409306) per well. ACE2 wells were stained with 1 ul anti-his-APC (Biolegend 362605). Cells were incubated with secondary for 25 min on ice, before washing 2× with cold FACS buffer. After the final wash, cells were fixed with 1% PFA and analyzed on a BD Canto II. Cycloheximide was added to inhibit protein production. Samples were ran in duplicate. Samples were normalized to the 5 min sample.

$$\% Change = {MFI}_{T = X} / {MFI}_{T = 5} * 100$$

Broad neutralization of SARS-related viruses by human monoclonal antibodies can be carried out as described in Wec et al. DOI: 10.1126/science.abc7424. Antibody binding activity to cell-surface SARS-CoV-2 S over time, as determined by flow cytometry. IgGs were incubated with cells expressing WT SARS-CoV-2 over the indicated time intervals. Binding MFI was assessed at 240 min for the samples. CR3022 is included for comparison.

Spike Shedding via FACS. 2E5 293T cells stably expressing SARS-2 spike were harvested and resuspened in 50 ul FACS buffer+20 uM cycloheximide in each well of a 96 well V bottom plate. Antibodies were diluted to 200 nM in FACS buffer+20 uM cycloheximide and aliquoted into a deep well 96 well plate. Ab and cell plates were incubated at 37° C. for 15 min to allow for equilibration. At each time point, 50 ul of ab mix was added to the cells for a final ab concentration of 100 nM in each well. Plate was incubated at 37° C. through out the experiment. After the final time point, the reaction was quenched with the addition of 150 ul cold FACS buffer followed by centrifugation in a pre-chilled centrifuge (4° C.) at 750 g for 5 min. Cells and wash buffers were maintained at 4C for the remainder of the experiment. Cells were stained with in 100 ul FACS buffer with 1 ul anti-hFc-APC (Biolegend 409306) per well. ACE2 wells were stained with 1 ul anti-his-APC (Biolegend 362605). Cells were incubated with secondary for 25 min on ice, before washing 2× with cold FACS buffer. After the final wash, cells were fixed with 1% PFA and analyzed on a BD Canto II. Cycloheximide was added to inhibit protein production. Samples were ran in duplicate. Samples were normalized to the 5 min sample.

$$\% Change = {MFI}_{T = X} / {MFI}_{T = 5} * 100$$

### Example 15—Synergy Calculation Using Neutralization Data

CI values at:

| Therapy | IC50 | IC95 |
|---|---|---|
| Ab 12 IgG + Ab 2-7 scFv-Fc | 0.67 | 0.68 |
| BsAb-HC | 0.10 | 0.12 |
| BsAb-LC | 0.32 | 0.32 |
| Ab 12 scFv-Fc + Ab 2-7 scFv-Fc | 0.99 | 0.97 |
| Ab 12/Ab 2-7 Tan | 0.33 | 0.33 |
| Ab 2-7/Ab 12 Tan | 1.61 | 1.34 |

CompuSyn was used to calculate the combination index (CI) for each therapy and extrapolated for various doses. CI values>1 indicates antagonistic interactions, values<1 indicate synergistic interactions, and values=1 indicate an additive effect.

### REFS FOR THIS EXAMPLE

T.-C. Chou, N. Martin, CompuSyn software for drug combinations and for general dose-effect analysis, and user's guide. ComboSyn, Inc. Paramus, NJ 2007. [www.combosyn.com] (2007).
T. C. Chou, P. Talalay, Quantitative analysis of dose-effect relationships: the combined effects of multiple drugs or enzyme inhibitors. Adv. Enzyme Regul. 22, 27-55 (1984).
T. C. Chou, Theoretical basis, experimental design, and computerized simulation of synergism and antagonism in drug combination studies. Pharmacol. Rev. 58, 621-681 (2006).

### Example 16—Design of Potent Human Anti-SARS-CoV-2 Spike Bispecific Antibodies with Synergistic Activity

Neutralizing antibodies are a promising approach to treat emerging viral pathogens. However scientific barriers to their use against SARS-CoV-2 remain formidable due to variability in circulating strains in human and animal species and their propensity to undergo neutralization escape. Antibody cocktails are being employed to mitigate these challenges, but alternative strategies are needed. Here we used our human naïve phage library to isolate a panel of antibodies, two of which were engineered into bispecific antibodies that resulted in distinct synergistic or additive effects on binding and neutralization activity. Promising results in mouse-adapted virus challenge studies were further confirmed by challenge of hACE2 mice and post-infection treatment of golden hamsters. These next generation anti-SARS-CoV-2 spike bispecific antibodies offer a therapeutic strategy for prevention and treatment of COVID-19.

### Introduction

Coronaviruses (CoVs) are named for their crown like spike proteins and have long been a part of our virus exposure history. Various endemic strains circulate worldwide among human and animal populations, causing a range of respiratory and gastrointestinal illnesses. In contrast, emerging epidemic CoVs undergo continuous zoonotic transfers from bats to other mammalian hosts, but such transfers seldom flourish due to constraints on their interspecies adaptation. In rare instances, successful interspecies adaptation does occur from intermediate hosts to humans, as we saw previously with the regionally localized epidemics of SARS-CoV in 2002-2003 and the ongoing outbreaks of MERS-CoV since 2012. In 2019, new coronavirus (SARS-CoV-2) made the leap into humans and rapidly developed into a catastrophic global pandemic, that has to date infected over 68 million people worldwide and caused over 1.5 million deaths (1). Additionally, The World Bank estimates that the worldwide GDP will contract by 5.2% in 2020, the deepest recession in decades (2). As cases continue to accumulate at nearly an exponential rate, it is challenging to resume any sense of normalcy until there are effective vaccine and therapeutic options.

The spike (S) glycoprotein is a homotrimeric glycoprotein decorating the surface of CoV particles. It is responsible for receptor recognition, viral fusion, tropism and pathogenesis and is recognized as the principle target for potent neutralizing antibodies (nAbs) (3-6). The S glycoprotein is composed of two subunits, S1 contains the receptor binding domain (RBD) and is responsible for binding ACE2, while S2 is responsible for membrane fusion (7-9). Numerous groups have isolated nAbs against SARS-CoV-2 from convalescent patient B cells, with Eli Lilly's nAb and Regeneron's nAb cocktail recently receiving emergency use authorization to treat high risk adults and pediatric patients with mild to moderate COVID-19 in early infection (10-12).

Monoclonal antibody (mAb) therapies provide protection from viral infection by inhibiting viral binding and entry into target cells, and unlike a vaccine are not dependent on initiating an endogenous response from the host (13). While traditional antibody (Ab) therapies were often designed using a single nAb, current anti-viral therapies often combine two Abs into a single therapeutic agent. This is due to the virus's ability to mutate from the selective pressure of a single Ab, leading to the generation of mutant strains that are immune to the original therapy. Ab cocktails avoid this scenario by utilizing nAbs targeting different epitopes, making it more difficult for the virus to escape without suffering from a loss of viral fitness (14). While Ab cocktails are attractive therapeutic options, the need for multiple nAbs significantly increases the cost and time of production, both of which are crucial during a fast-moving global pandemic. multiple

Bispecific antibodies (BsAbs) combine the antigen binding domains from two mAbs onto one framework and provide an alternative to producing two separate mAbs for cocktail therapy. BsAbs are classified in one of two categories, IgG like or non-IgG like. IgG-like BsAbs contain an Fc domain allowing them to engage effector functions and constructs range from the original asymmetric knob-in-holes, to multivalent IgG-scFv fusions, to the highly optimized cross-over dual variable (CODV)-Igs (15-18). Non-IgG like BsAbs include diabodies and dual-affinity re-targeting antibodies (DARTs) and are built using linked variable regions (19, 20). Smaller and more agile, these BsAbs provide rapid biodistribution and organ penetration. However their size and lack of an Fc region allows them to be promptly cleared by the kidneys (21). While few BsAbs are currently approved by the FDA, anti-viral BsAbs have lagged behind but are under development for HIV, influenza, and Ebola and many of these exhibit increased potency compared to the parental antibodies (22-25). Here we report on the design and activity of several anti-SARS-CoV-2 BsAbs that have been structurally mapped on the spike RBD. Our results show synergistic binding and neutralization activity and protection against in vivo SARS-CoV-2 challenge. These BsAbs provide new prophylactic and therapeutic candidates to combat COVID-19.

### Results

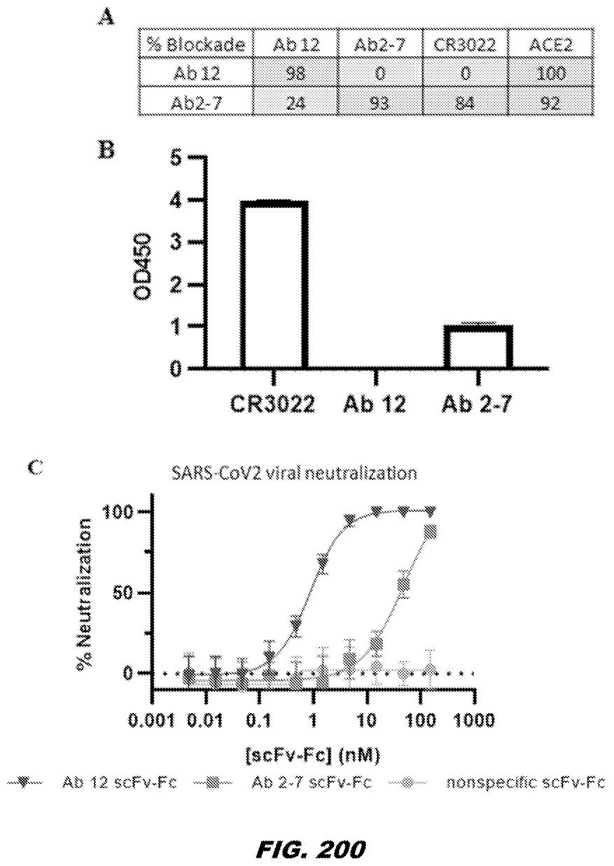

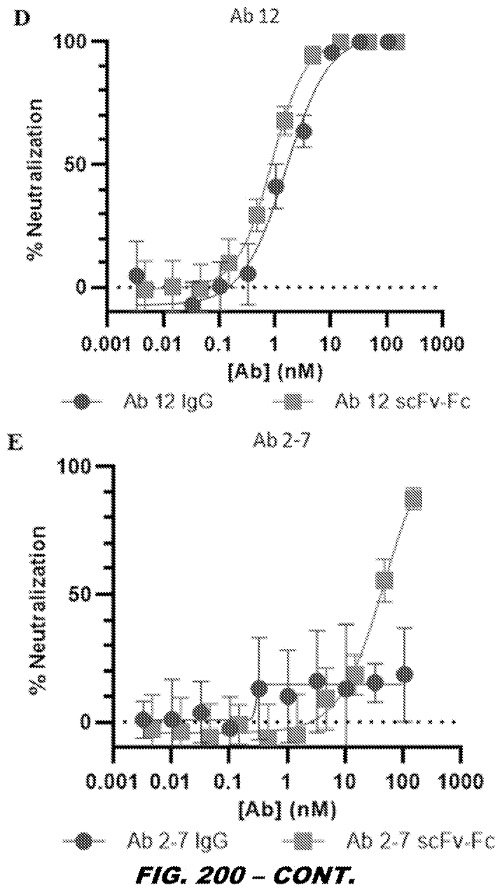

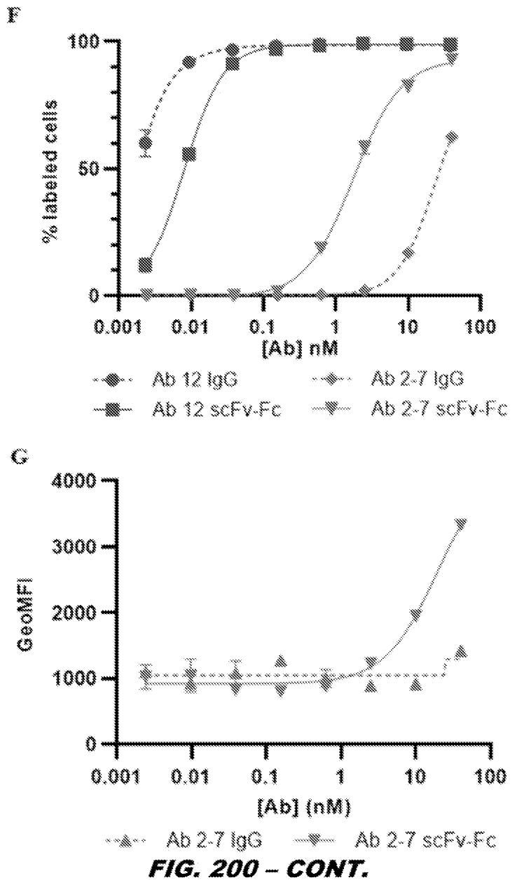

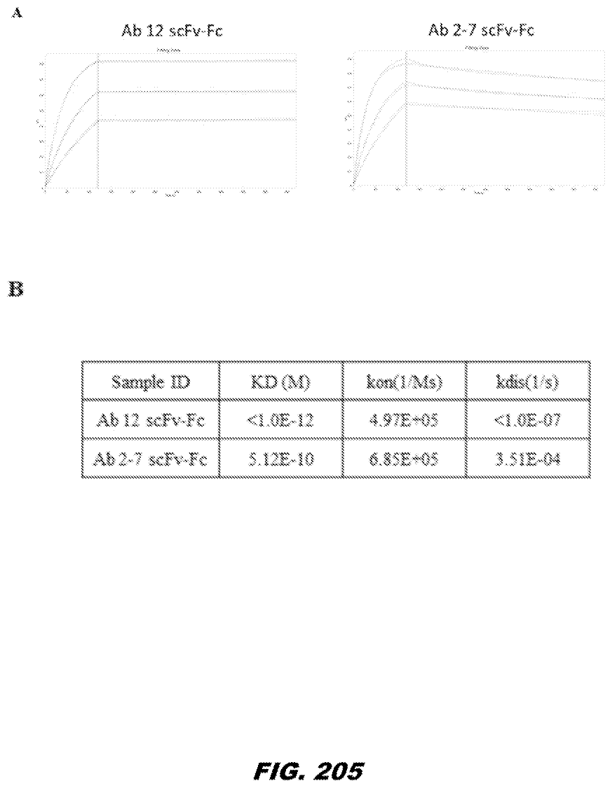

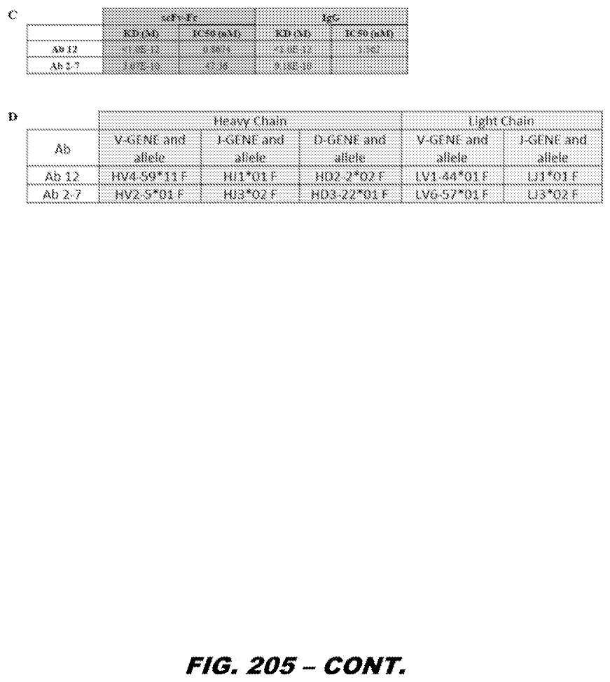

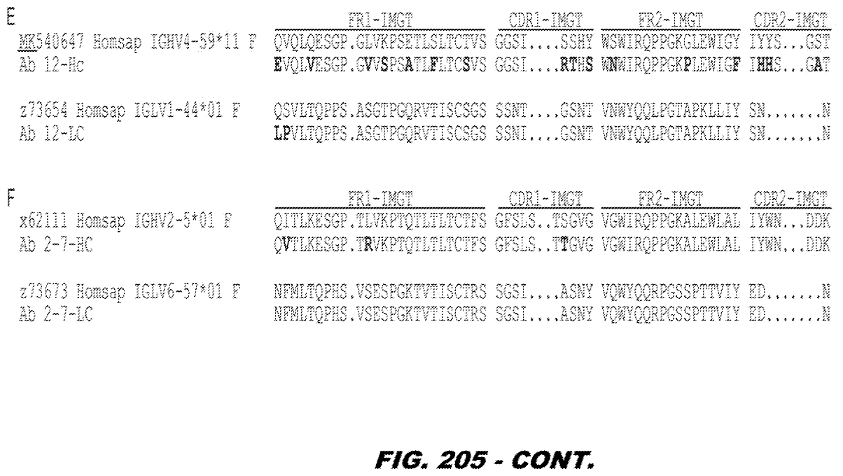

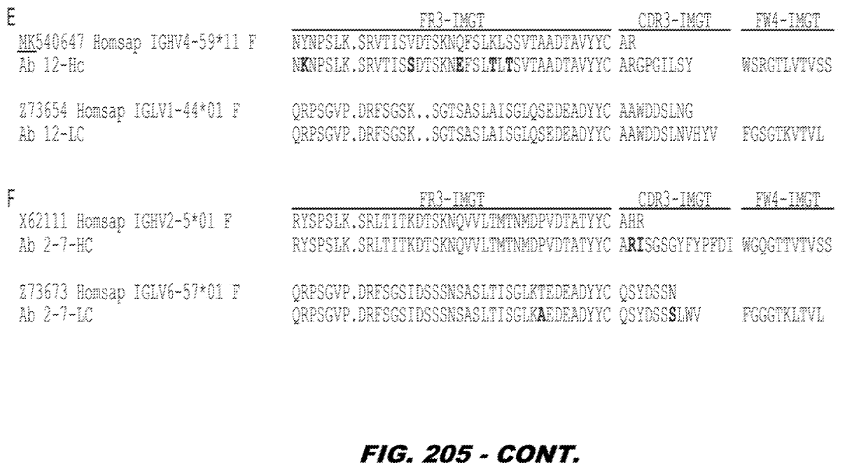

Discovery, characterization and analysis of Ab 12 and Ab 2-7 binding. Abs 12 and 2-7 were discovered via phage panning of our 27 billion-member naïve phage library against recombinant SARS-CoV-2 S1 and the receptor binding domain (RBD). Kinetic parameters for Ab 12 and 2-7 scFv-Fcs were measured via biolayer interferometry (BLI) against recombinant RBD and revealed that both had nanomolar affinity to the SARS-CoV-2 spike (FIG. 205A and FIG. 205B). Initial epitope binning was performed with the scFv-Fcs via competition with ACE2 and CR3022, as they bind opposite ends of the RBD(26). This screening revealed that while both Ab 12 and 2-7 blocked ACE2 binding, only Ab 2-7 inhibited CR3022 binding to the RBD (FIG. 200). In addition to competing with both ACE2 and CR3022, Ab 2-7 is able to bind SARS-CoV spike protein (FIG. 200B), demonstrating that it binds the more conserved CR3022 epitope while sterically blocking ACE2 binding to the spike. Because these Abs were found to be non-overlapping they were chosen for further evaluation and engineering.

Abs 12 and 2-7 were tested for SARS-CoV-2 neutralization via plaque reduction neutralization tests (PRNT) and recombinant nLuc viruses. Both Abs neutralized SARS-CoV-2, although Ab 12 scFv-Fc was significantly more potent with an IC50 of 0.86 nM compared to 47.36 nM for Ab 2-7 in PRNT assays (FIG. 200C). With confirmation that both Abs neutralize live virus, they were converted to IgG. We observed insignificant changes in binding kinetics (FIG. 205C). However, when the IgG and scFv-Fc formats were tested in parallel neutralization assays, we observed that Ab 12 IgG and scFv-Fc showed comparable neutralization while surprisingly Ab 2-7 IgG lost neutralization activity (FIG. 200E).

To reconcile this shift in neutralization, FACS binding with the IgG and scFv-Fc constructs was performed with 293T cells stably expressing surface bound SARS-CoV-2 spike protein. Ab 12 IgG showed a shift to the left on the dose response curve compared to the original scFv-Fc, while the Ab 2-7 IgG showed a shift to the right in spike binding (FIG. 200F). The geometric mean fluorescence intensity (geoMFI) for Ab 2-7 binding shows a stark difference in fluorescence intensity, as IgG binding is barely above the background at even the highest concentration tested, (FIG. 200G) demonstrating that very little Ab 2-7 IgG is binding to the spike on the cells.

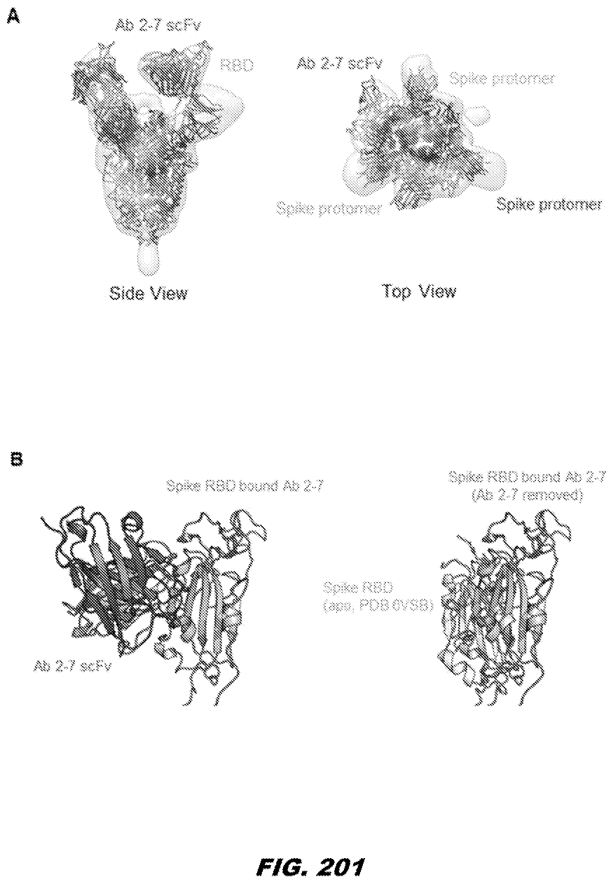

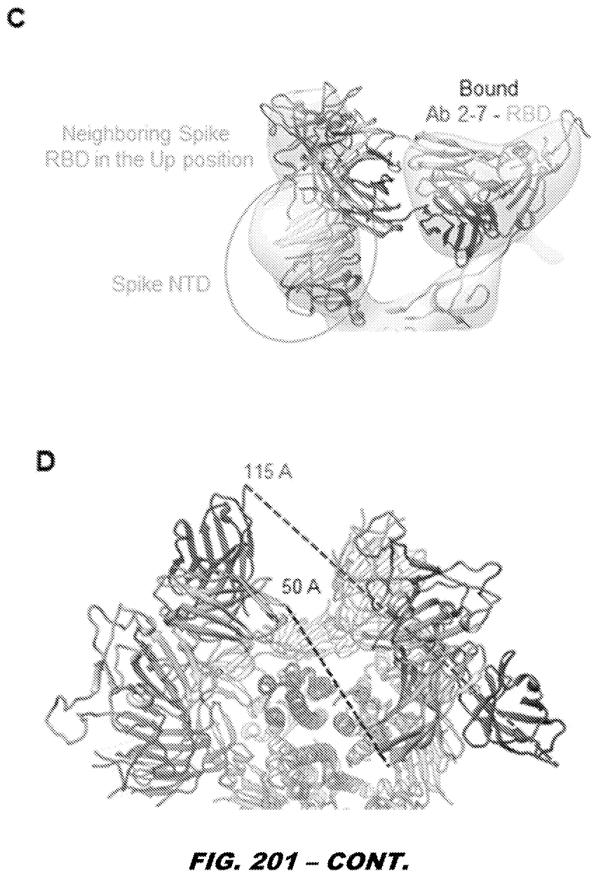

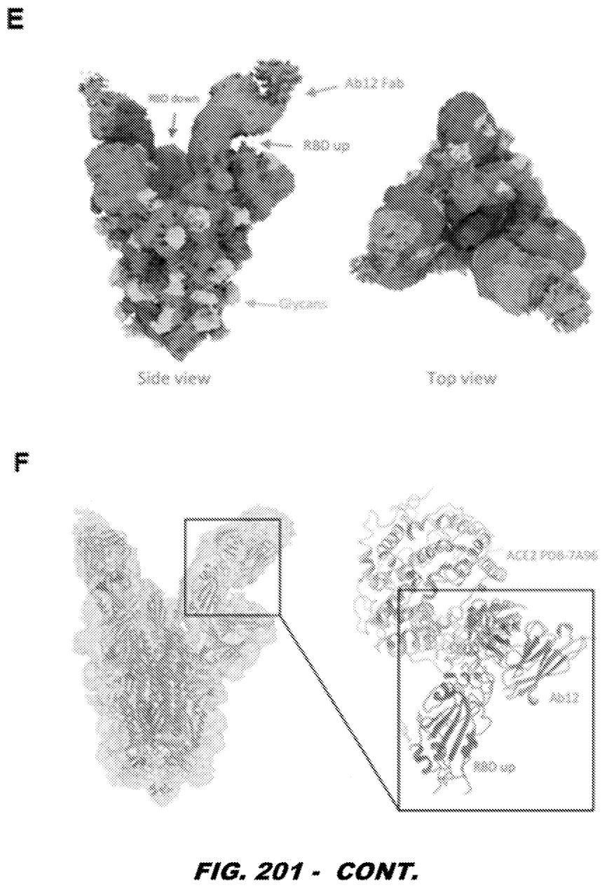

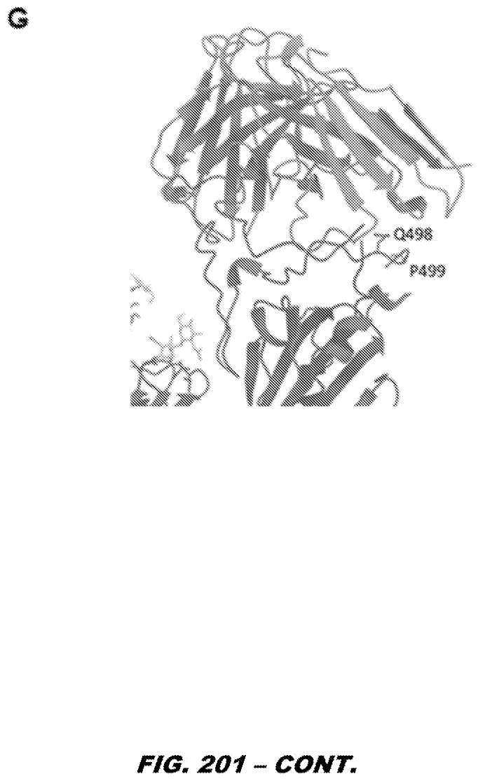

Structural solutions of Ab 2-7 and Ab 12 binding to spike. The observation that Ab 2-7 IgG strongly binds to RBD but not to full length spike demonstrates that there is a structural constraint and that only the smaller and more flexible scFv-Fc is able to bind whereas the large IgG cannot. To further explore the root cause of this finding, the structure of Ab 2-7 scFv bound to stabilized spike protein was solved by Cryo-EM. The electron density maps were fit with two solved structures (PDB-6XS6 and 6W41), allowing for resolution of different domains of the spike protein and identification of Ab 2-7 scFv molecules on at least two of the RBDs (FIG. 201A) (26, 27). Further analysis of the RBD-Ab 2-7 complex in FIG. 201B shows that Ab 2-7 scFv binds to a similar area and forces the RBD into the “up” conformation as reported by Yuan et al. with CR3022 (26).

Macroscopic analysis of the spike-Ab 2-7 complex also provides an explanation for loss of IgG neutralization. FIG. 201C reveals that due to the angle of binding, addition of the CH1/CL domains in the Fab/IgG can result in steric clashes with the RBD and NTD of a neighboring spike molecule, severely limiting the binding of the full IgG complex. We also examined the ability of the scFv-Fc to bind multiple spikes within a spike trimer by estimating the distance between the C-termini of neighboring Ab 2-7 scFv-Fcs under different conditions. Depending on the dominant chain that makes contact with the spike, the distance between the C-termini of two scFvs is circa 56 A to 117 A, the longer of which an scFv-Fc can be unable to span (FIG. 201D).

Cryo-EM structures were next solved for Ab 12 Fab in complex with the ectodomain of the SARS-CoV-2 spike at 3.6 Å resolution. The three-dimensional classification of the particles showed the presence of spike trimers in the pre-fusion conformation with one or two Ab 12 Fabs bound at once. As with Ab 2-7, Ab 12 also targets the RBD in an “up” configuration (FIG. 201E). Ab 12 seems to recognize an epitope focused on the receptor-binding ridge, partially overlapping the flat face of the RBD that is responsible for binding ACE2, confirming biochemical results that this antibody directly competes with ACE2 for binding (FIG. 201F).

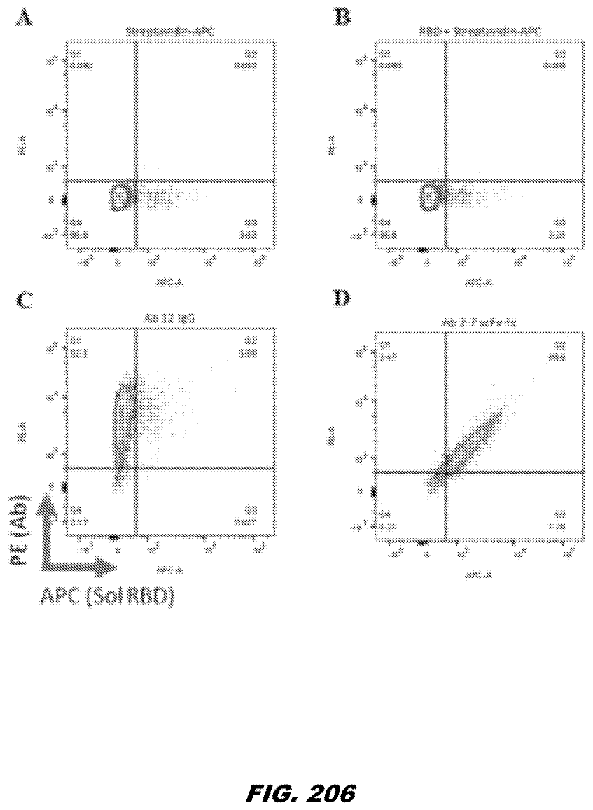

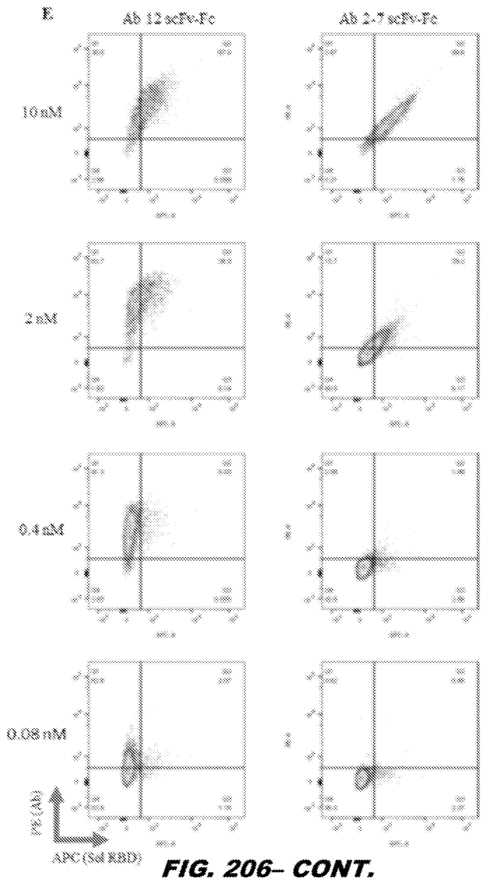

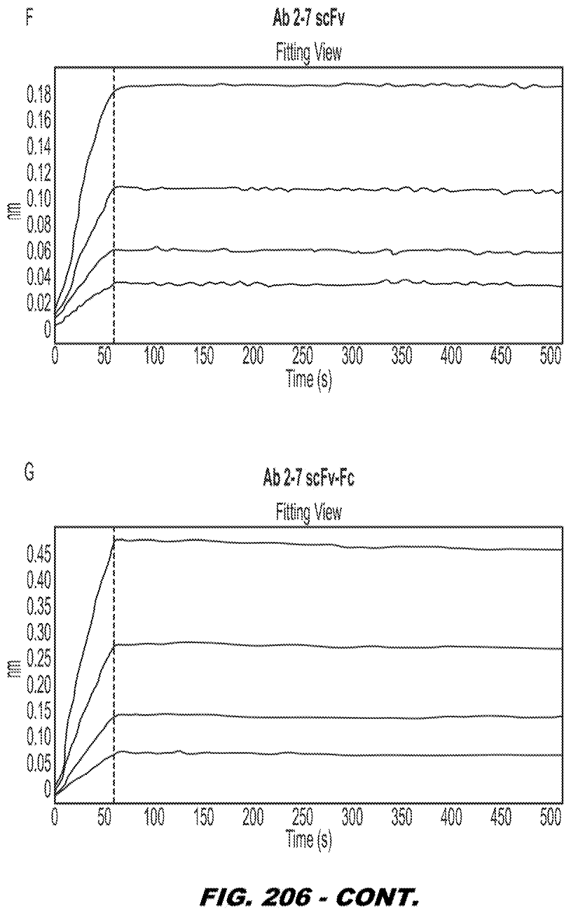

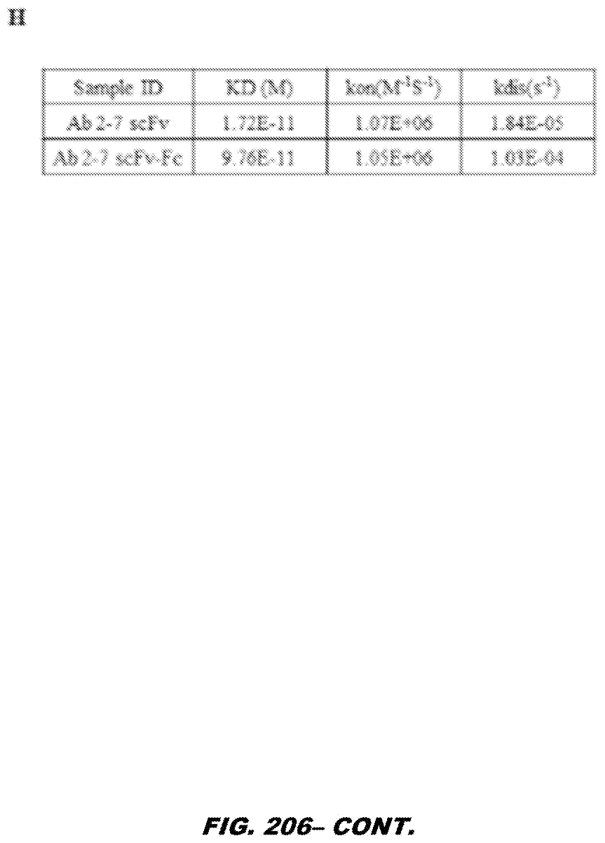

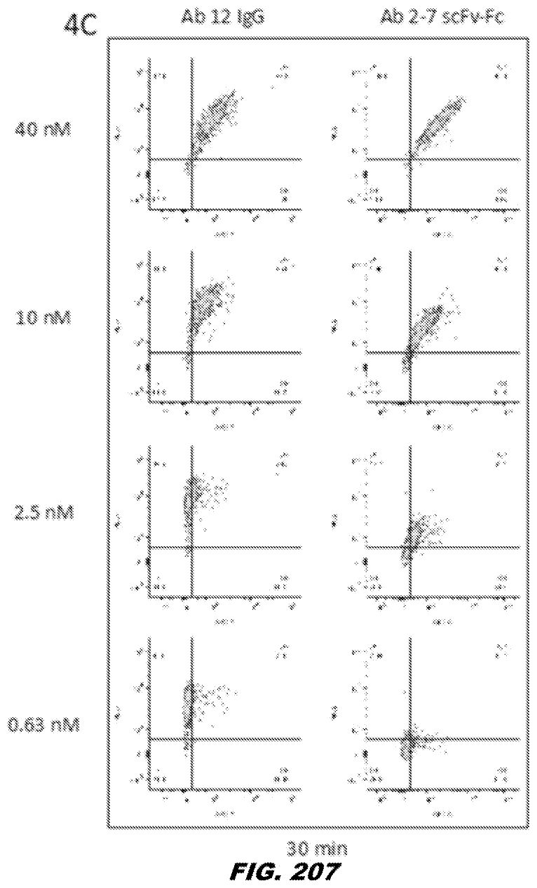

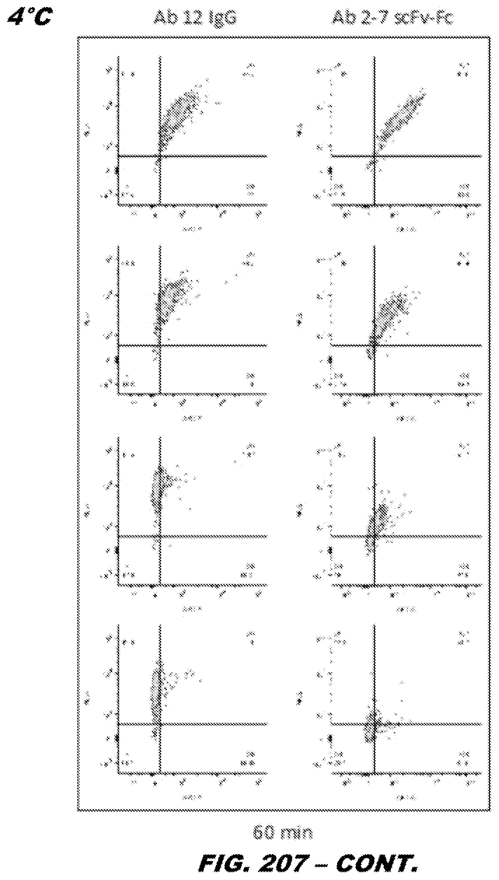

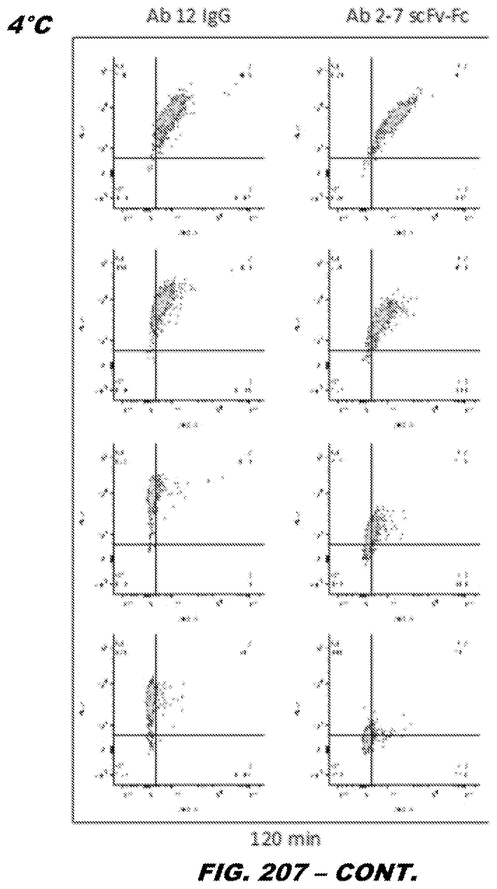

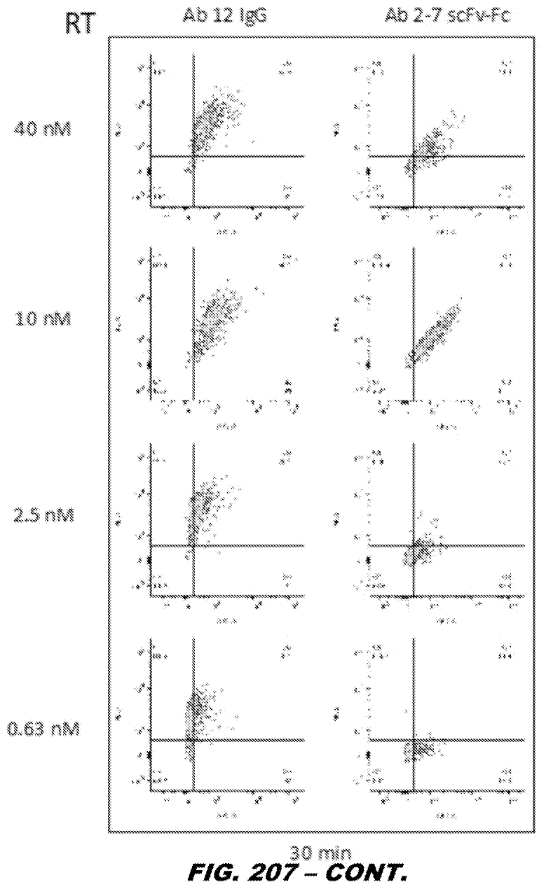

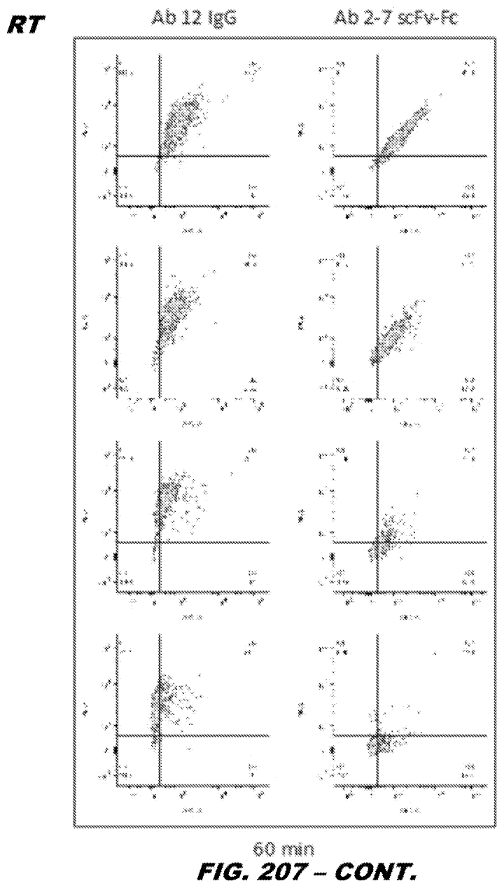

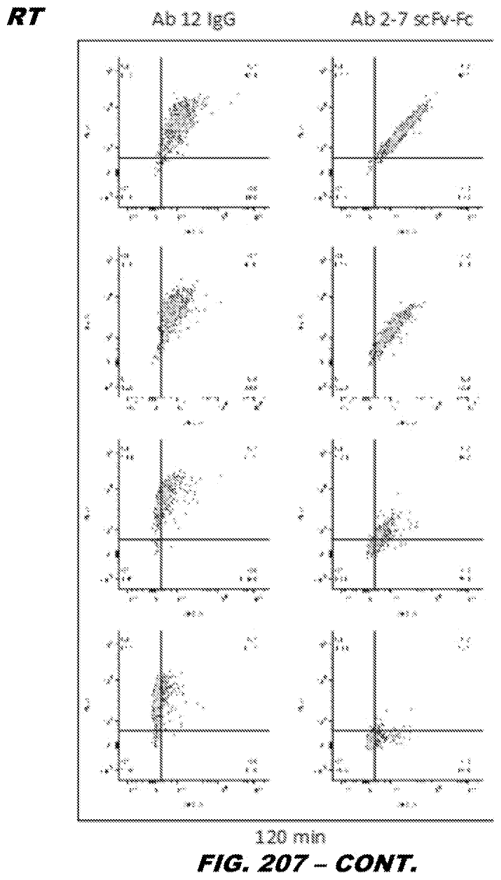

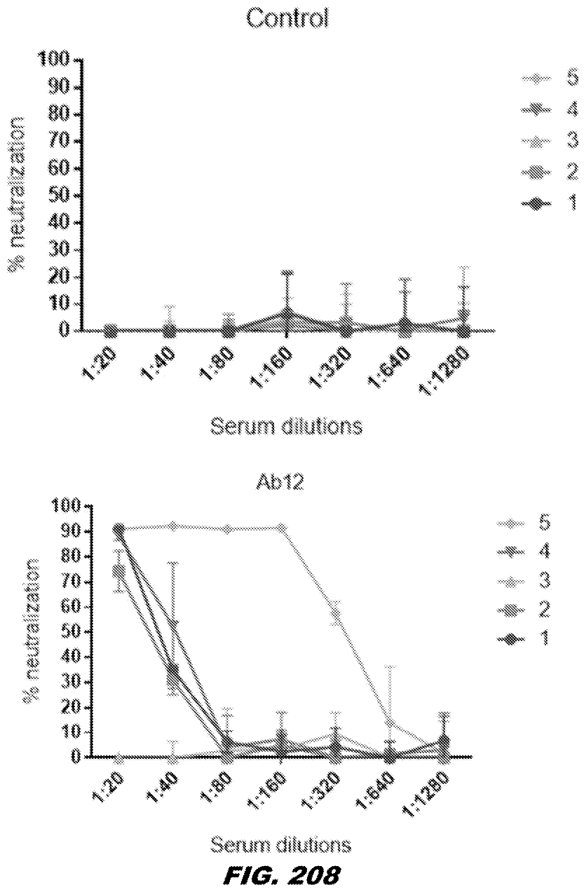

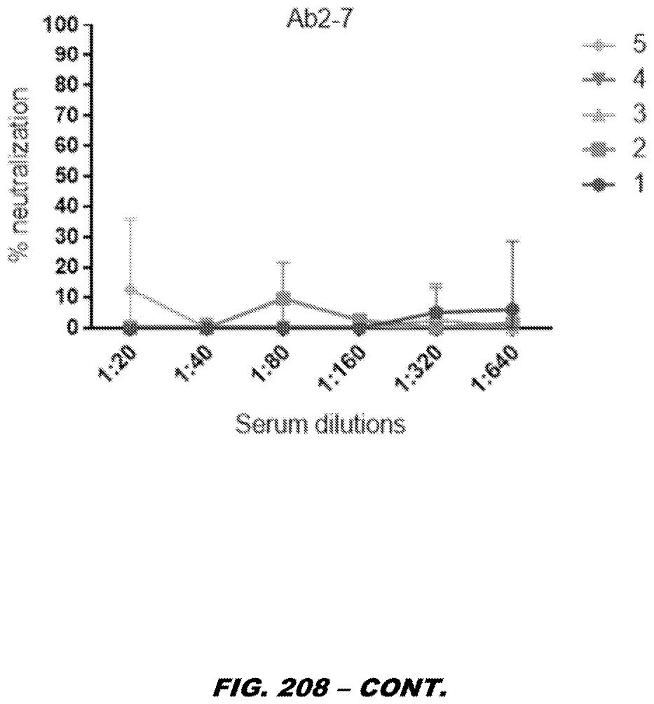

Mode of Ab 2-7 and Ab 12 binding. To further interrogate Abs 12 and 2-7's ability to simultaneously engage cell surface spike with both binding arms, we performed dual staining experiments with 293T-Spike cells and soluble RBD-biotin. FIG. 206C shows that Ab 12 IgG binds to the spike with both arms occupied such that there is no capture of soluble RBD. In contrast, FIG. 206D shows that Ab 2-7 scFv-Fc is only able to bind to the spike with one arm over a wide concentration range (FIG. 206E). This monovalent binding to cell surface spike by Ab 2-7 does not appear to be due to an avidity effect as both the monovalent Ab 2-7 scFv and bivalent Ab 2-7 scFv-Fc show similar binding kinetics (FIG. 206F-H). These binding experiments were also performed at different timepoints and temperatures to ensure that the binding studies were conducted under equilibrium conditions (FIG. 207). These results demonstrate that a physical constraint exists on the ability of the two Ab 2-7 scFv arms of the scFv-Fc construct to bind a second spike monomer and demonstrates that the heavy chain makes the predominant contacts with RBD (FIG. 201D).

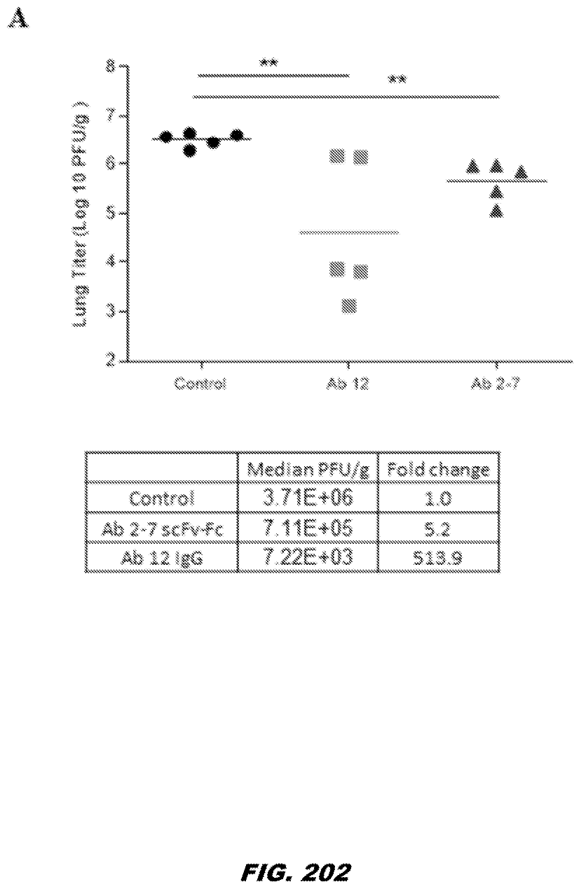

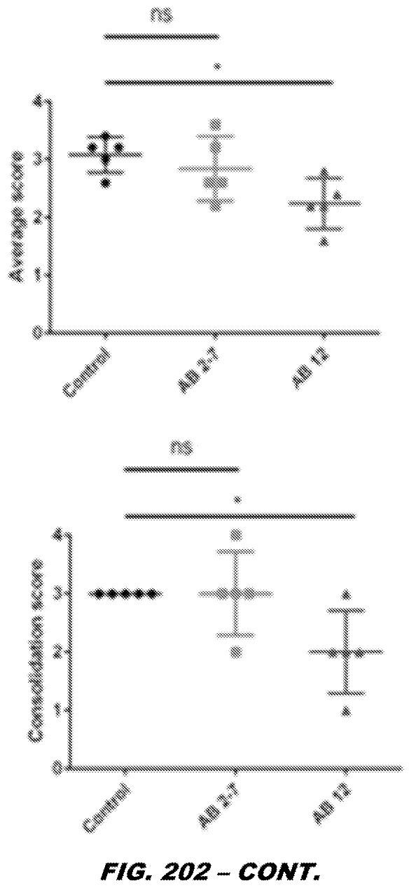

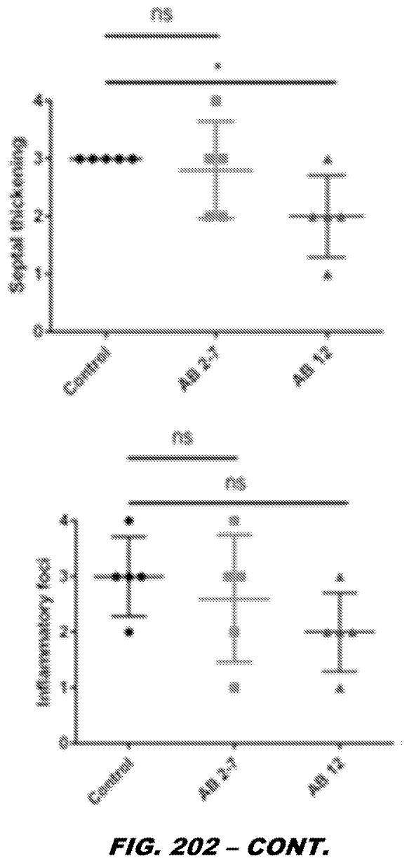

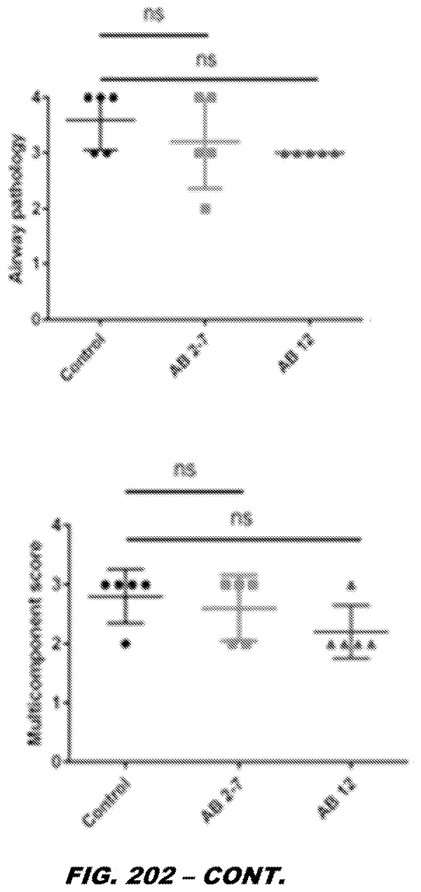

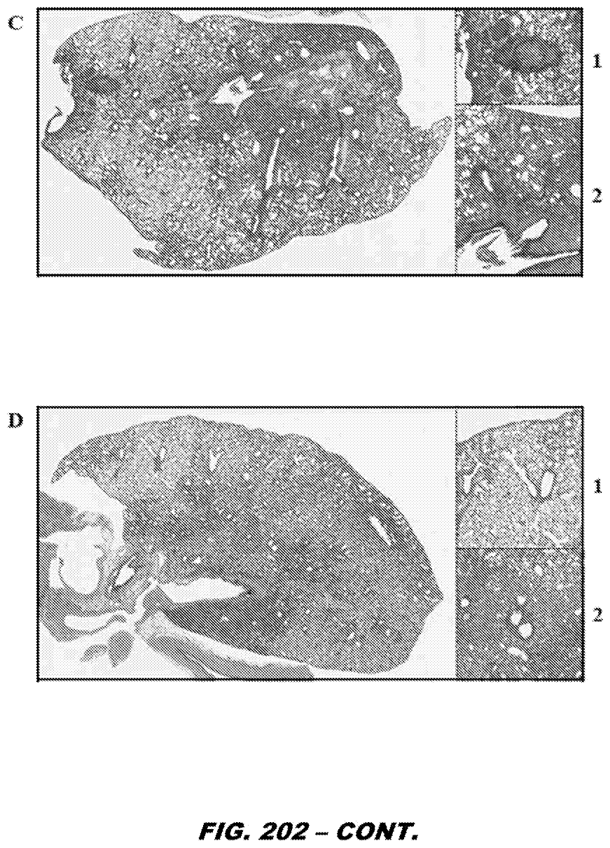

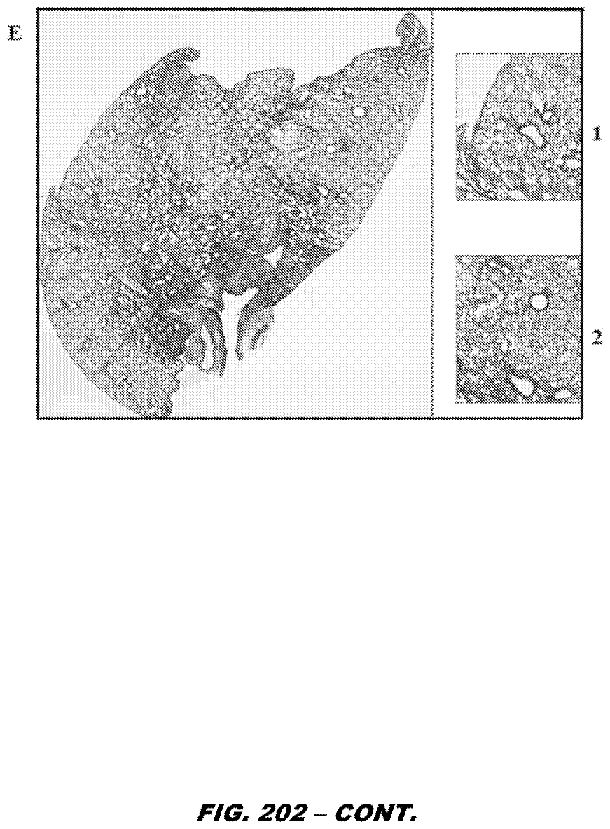

In vivo testing of Ab 12 IgG and Ab 2-7 scFv-Fc with Syrian golden hamsters. The in vivo biological activity of these Abs was next tested in Syrian golden hamsters, a model for severe COVID-19 (28, 29). Consistent with in vitro data, therapeutic treatment resulted in a significant decrease in the viral titers in their lungs, with Ab 12 IgG therapy resulting in >500-fold decrease in titer compared to untreated animals (FIG. 202A). Pathological analysis of the lung tissues showed that compared to control treated animals, Ab 12 treatment leads to marked reduction in consolidation, smaller foci of infiltration, and minimal perivascular cuffing (FIG. 202B, FIG. 202C& FIG. 202E). In contrast, Ab 2-7 scFv-Fc treatment resulted in only a 5-fold decrease in titer compared to control treated animals (FIG. 202A). Pathologic scoring demonstrated that Ab 2-7 treatment did not significantly reduce consolidation or inflammatory infiltration although a modest decrease in the number of inflammatory cells in the airways and in severity of the perivascular cuffing was observed (FIG. 202B-D). Thus, while Ab 12 IgG showed therapeutic benefit when used alone, only modest anti-viral effects were seen with Ab 2-7 scFv-Fc.

Design and rationalfor anti-SARS-CoV-2 spike BsAbs. The tethering of two Abs targeting different epitopes on the same protein has been shown to result in an increase in apparent Ab concentration as binding of one arm keeps the second arm localized around the second binding epitope(30). Additionally, increased valency has been shown to significantly improve kinetic properties and potentially increase the synergistic effect of the Abs(31-34). We reasoned that as Ab 12 and 2-7 target distinct, non-overlapping epitopes on the RBD, they can provide ideal candidates for BsAb engineering.

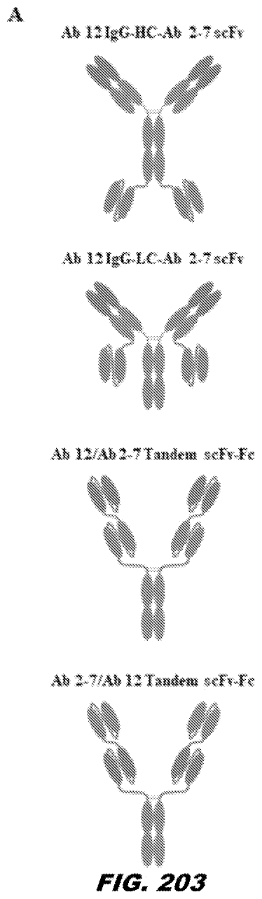

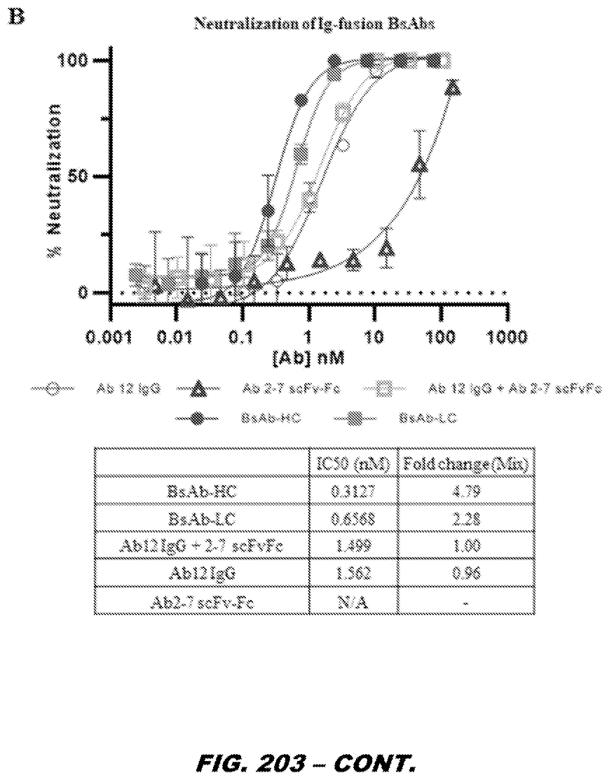

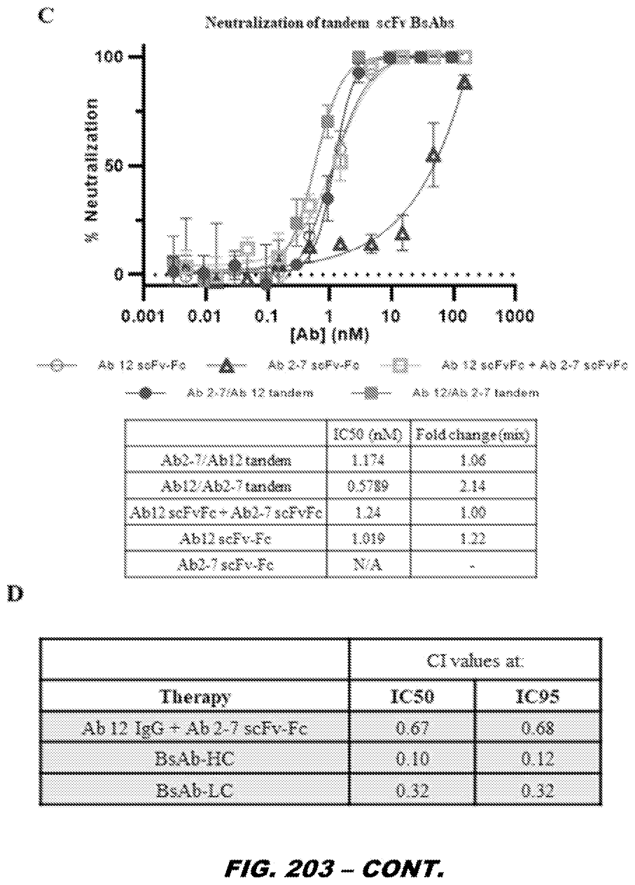

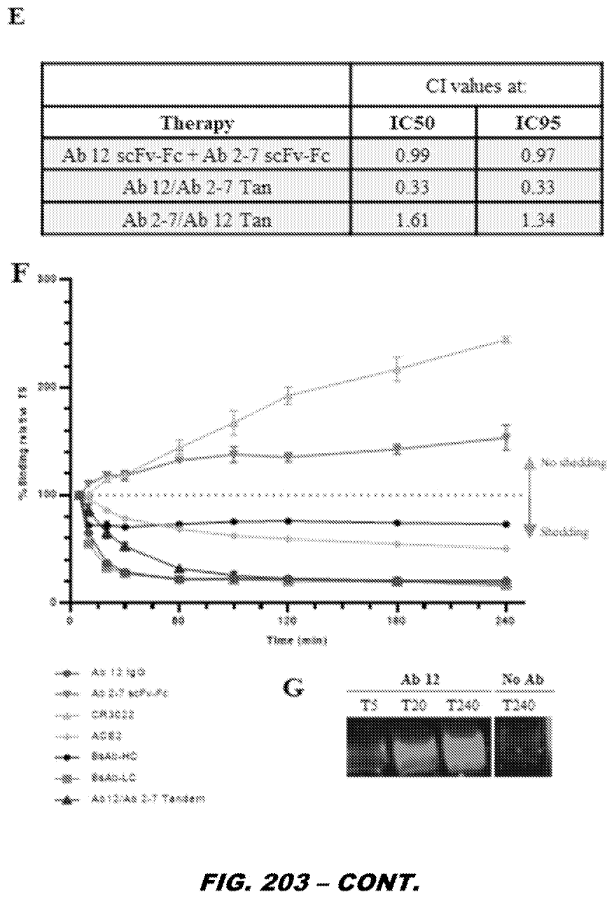

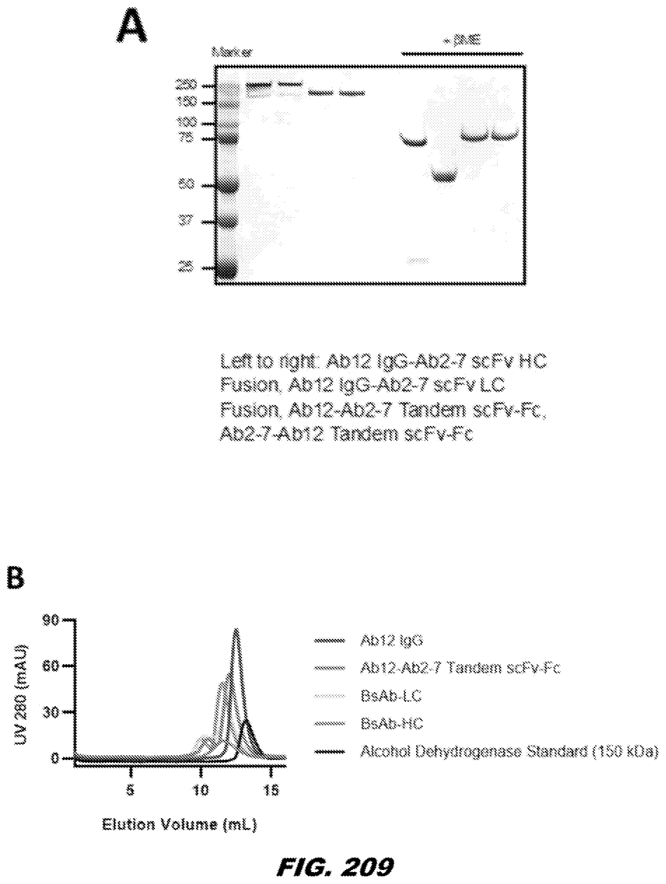

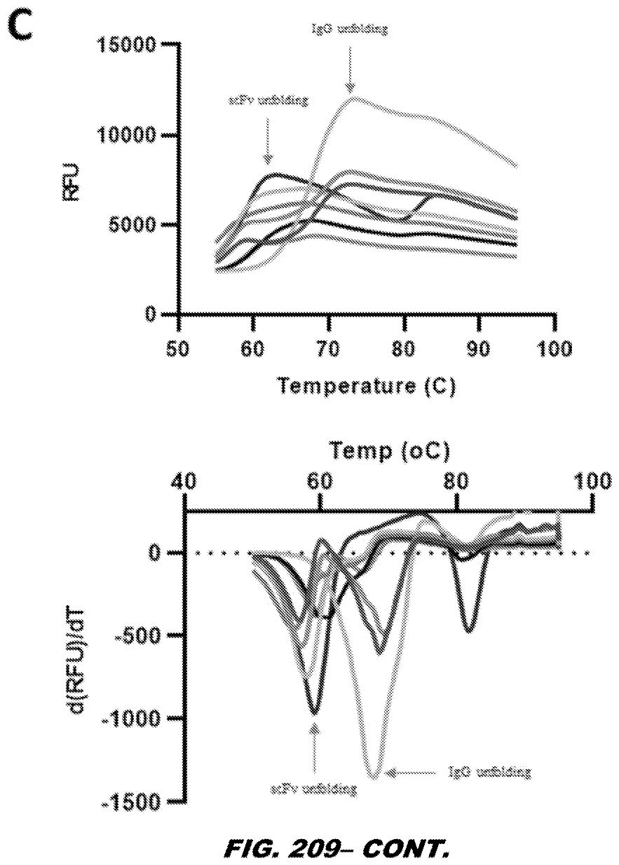

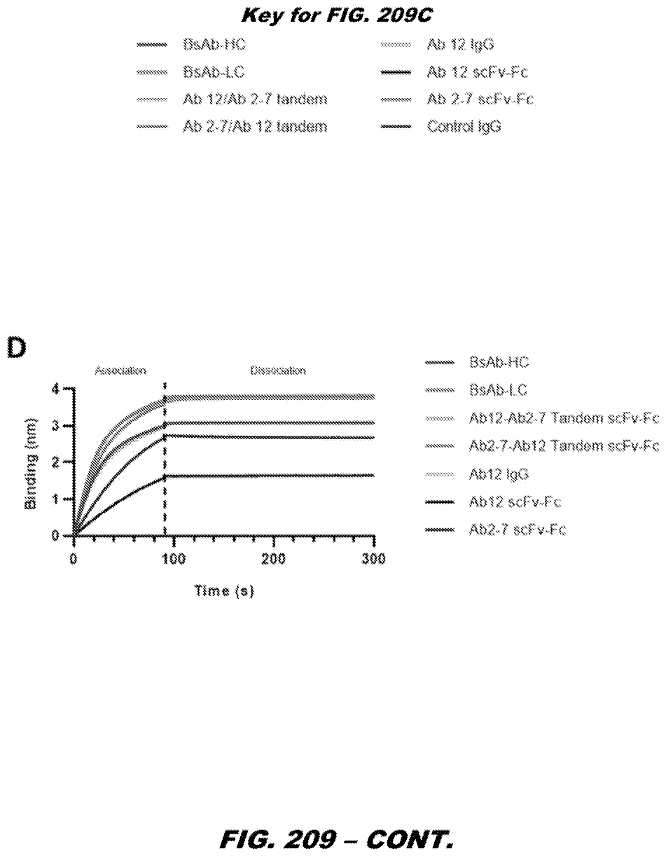

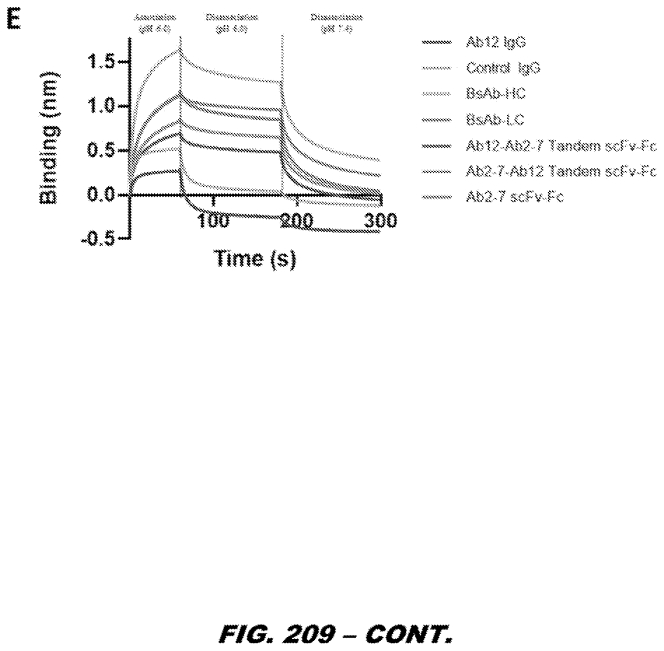

Three tetravalent, bispecific Ig-like scaffolds were used to construct four BsAbs: two IgG fusions and one tandem scFv-Fc. These scaffolds were designed to accommodate different binding site orientations and distances between RBD epitopes (FIG. 203A). Comprehensive biochemical characterization of these 4 BsAbs was performed (FIG. 209).

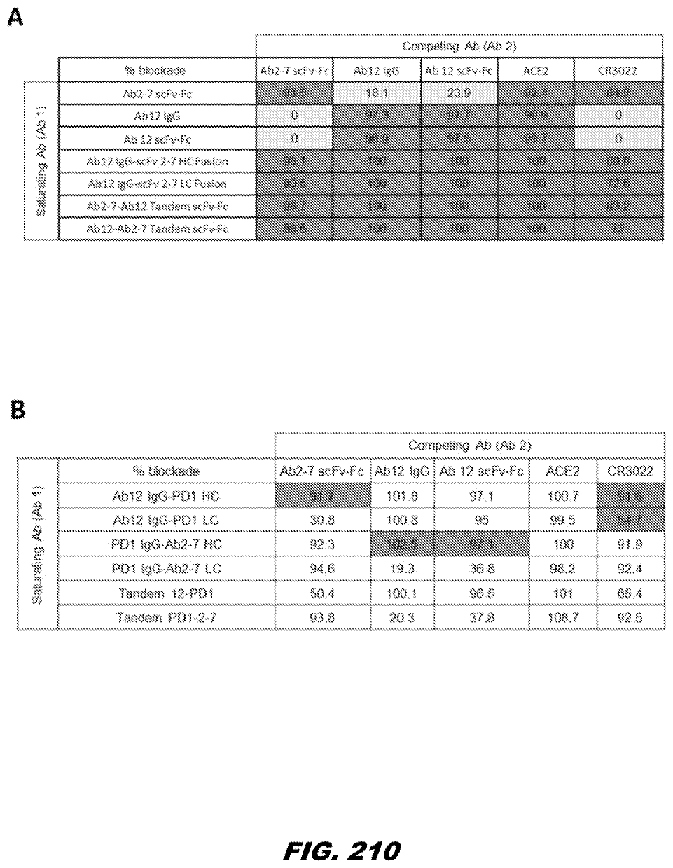

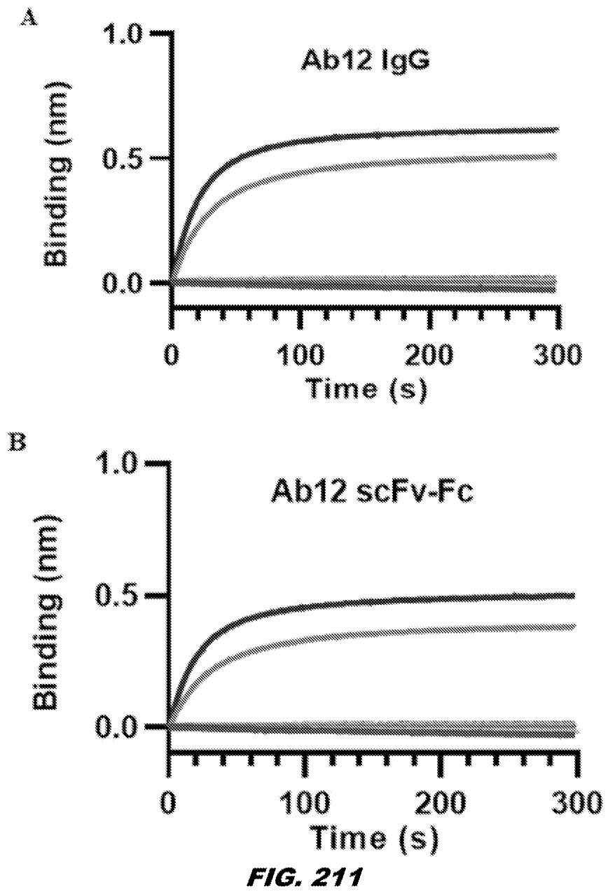

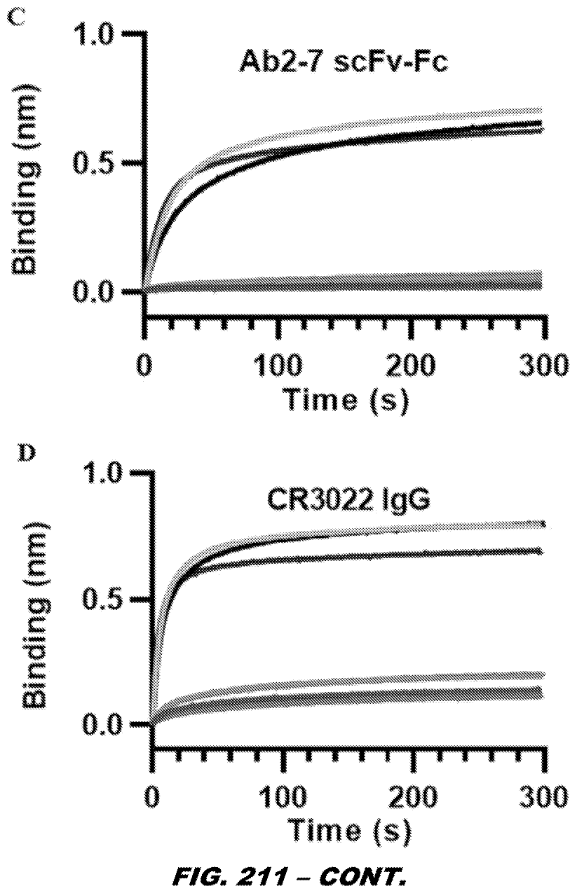

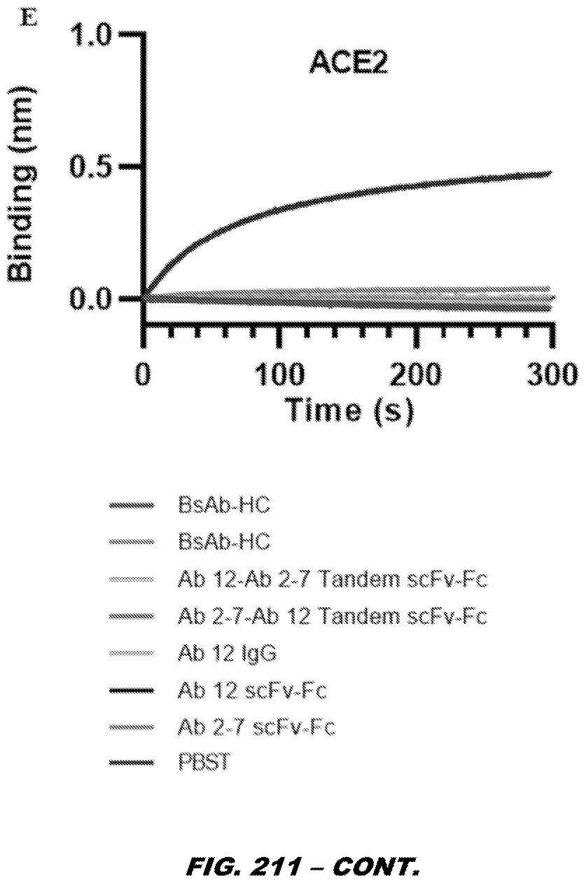

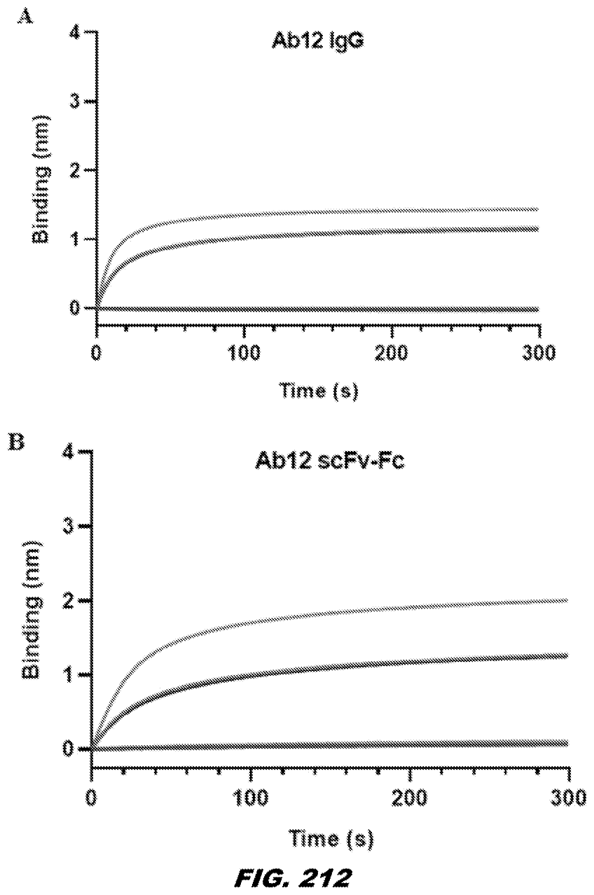

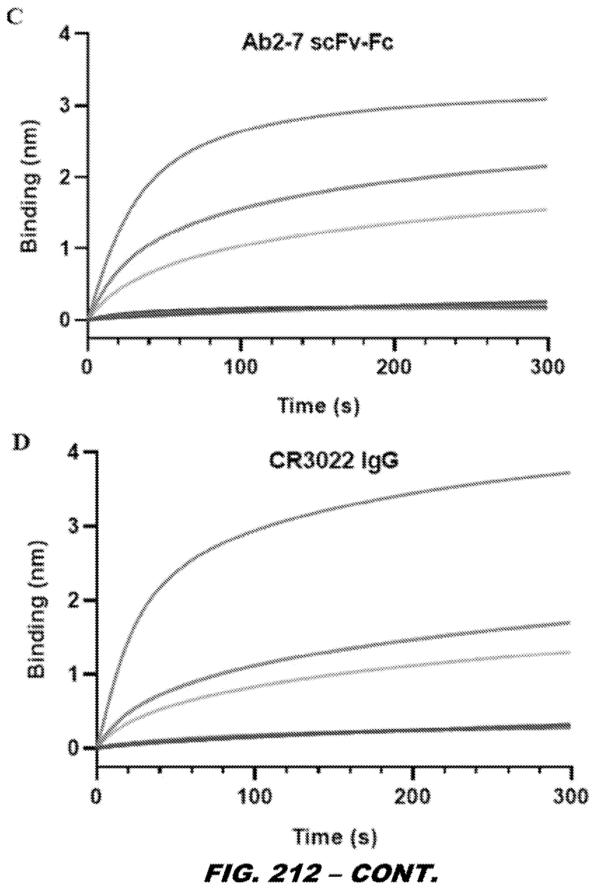

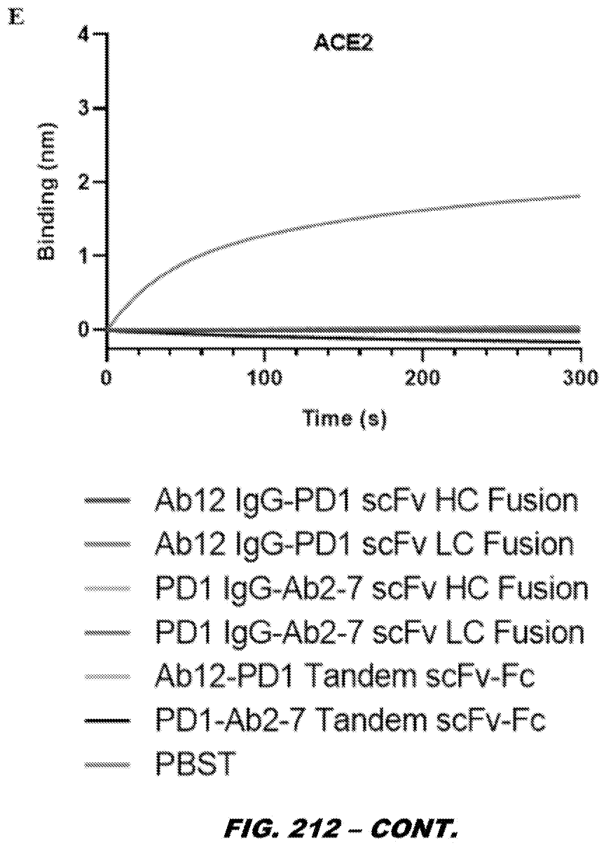

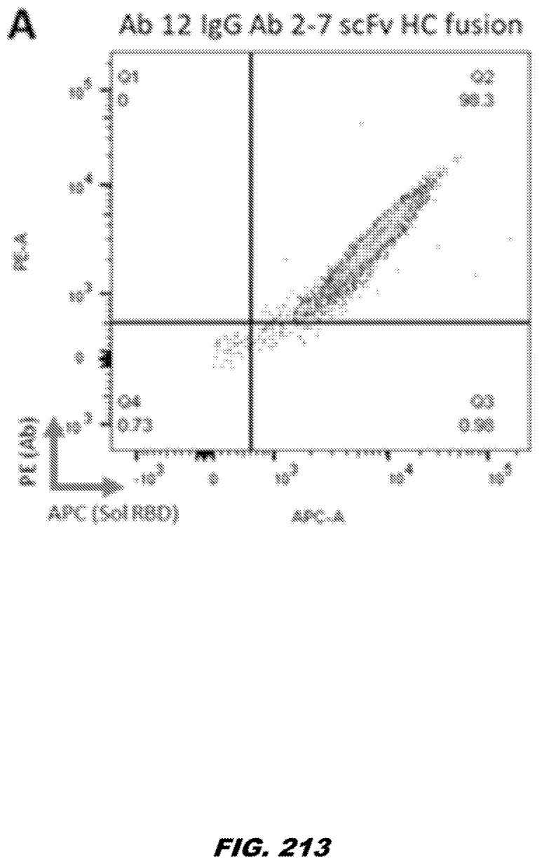

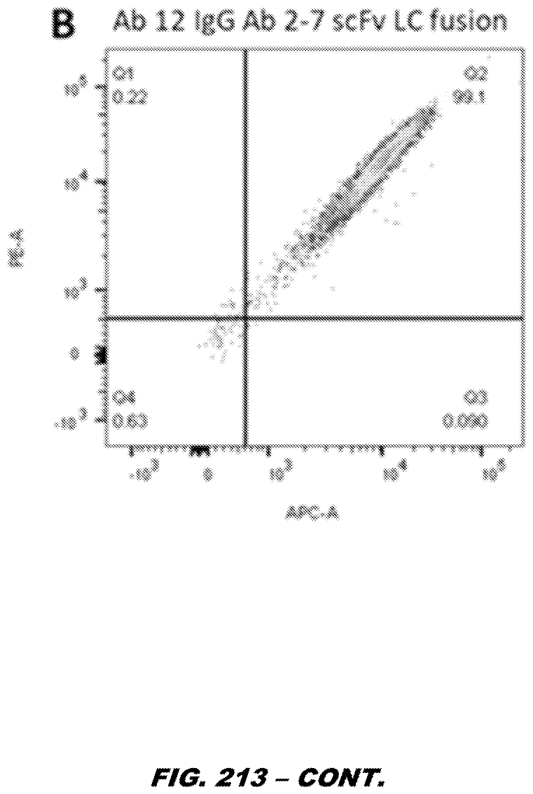

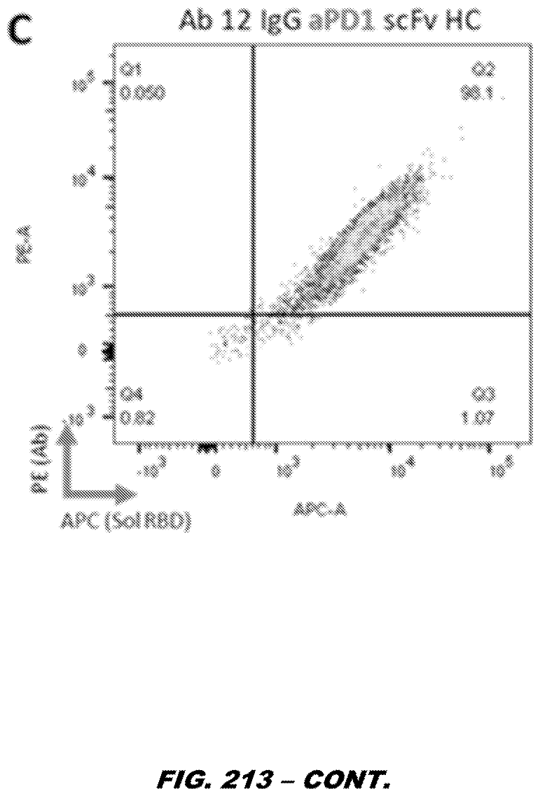

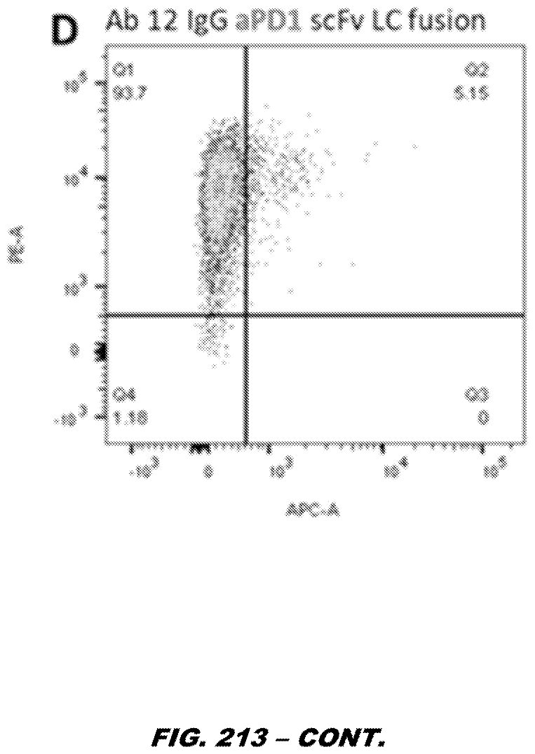

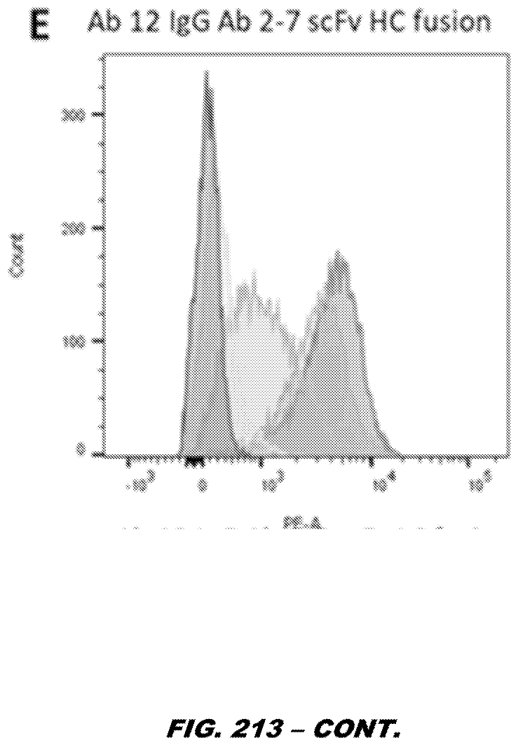

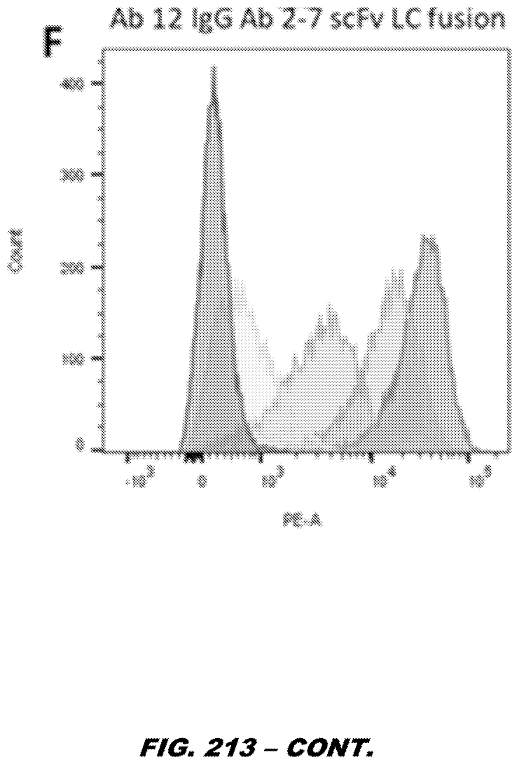

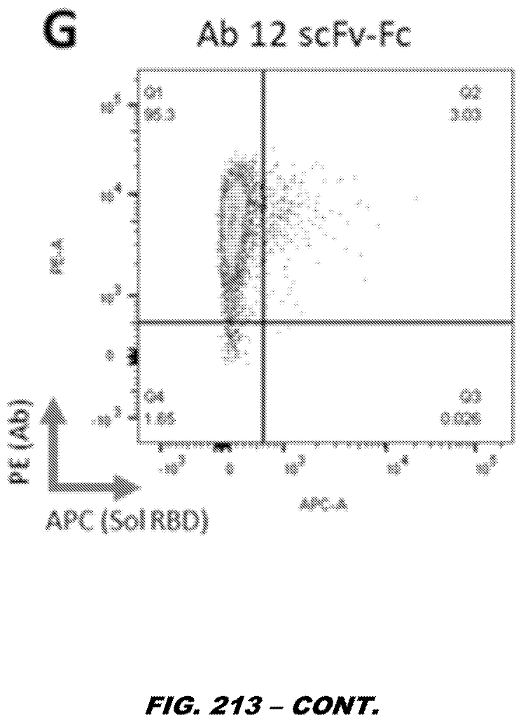

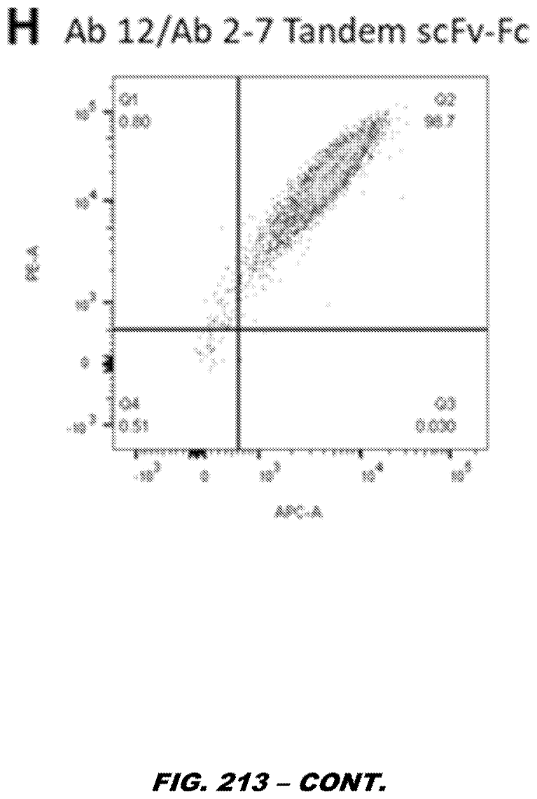

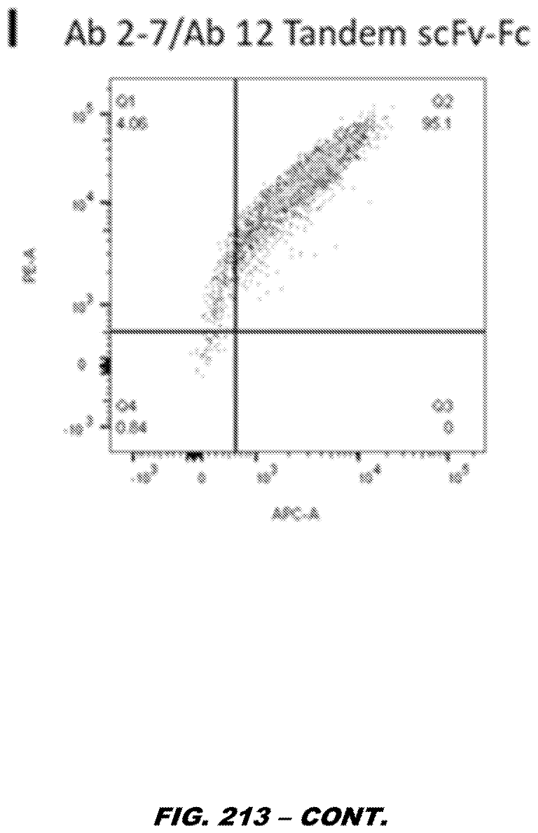

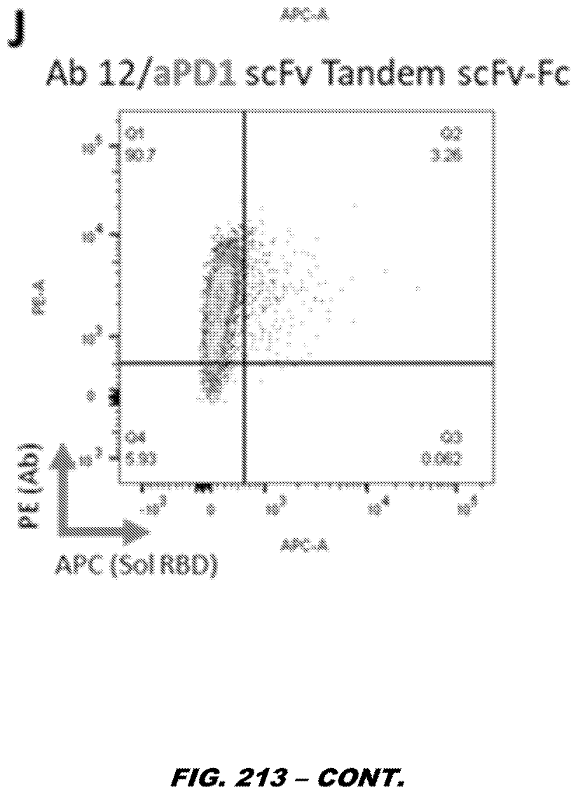

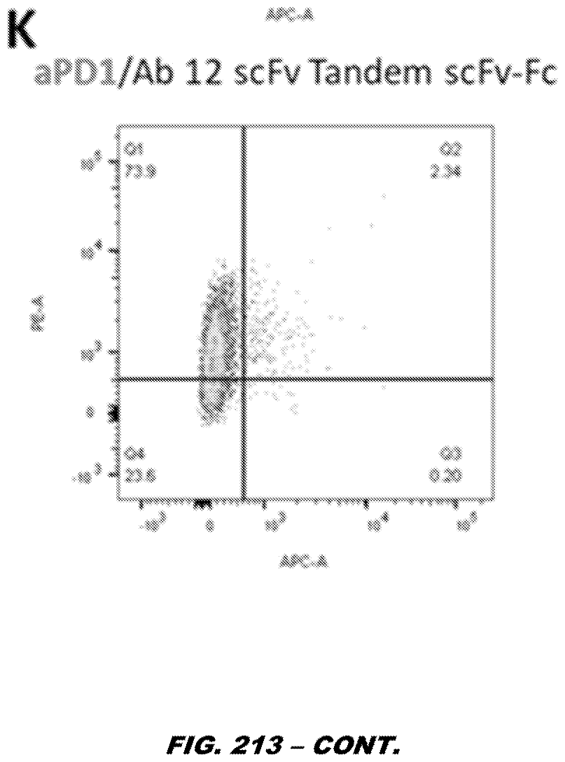

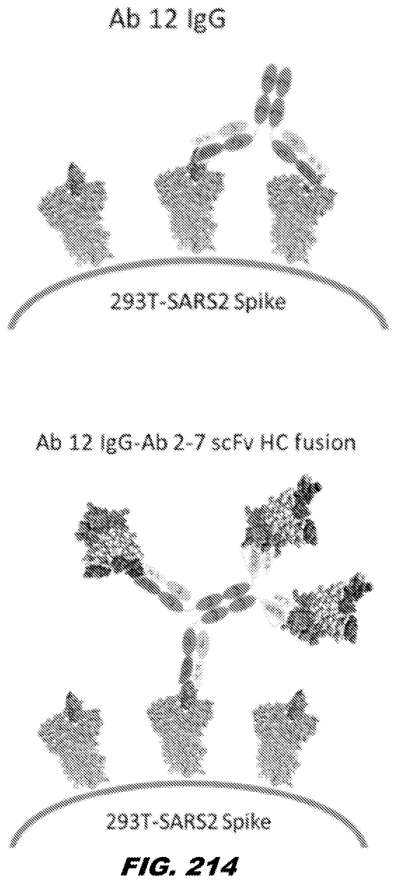

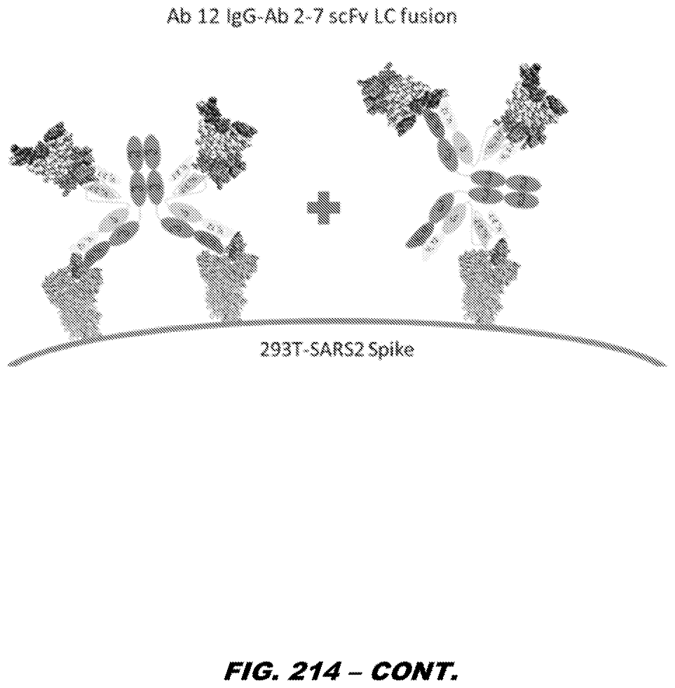

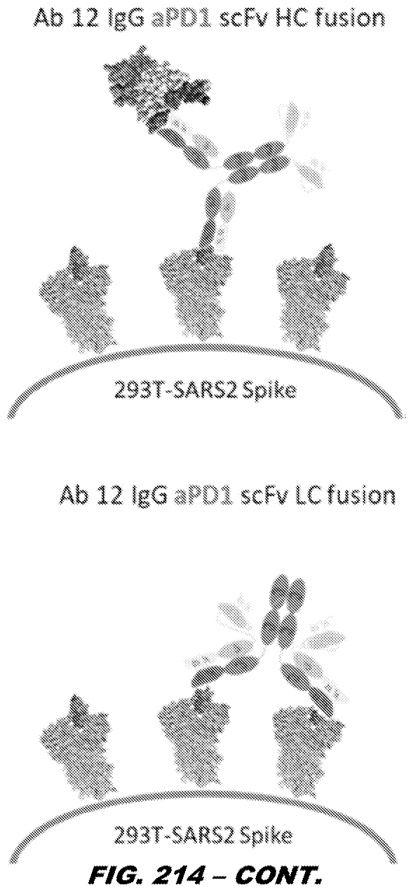

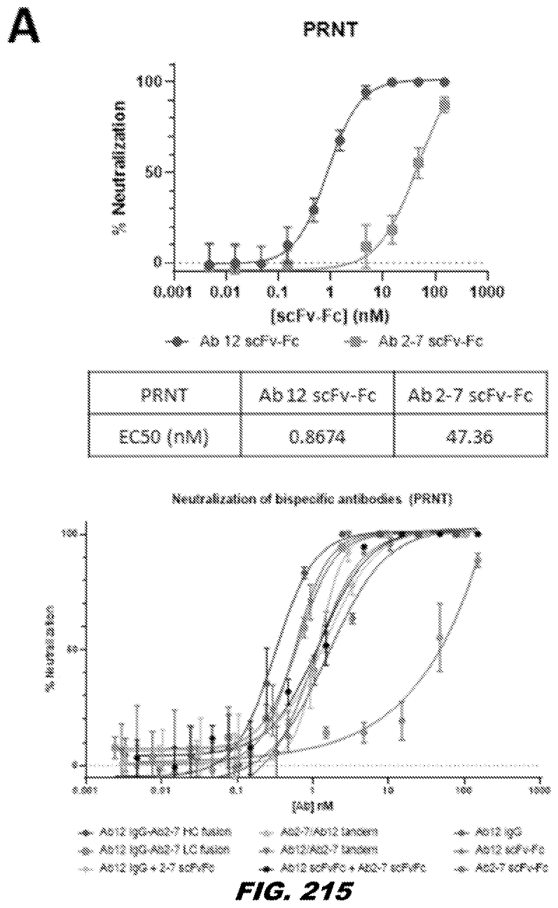

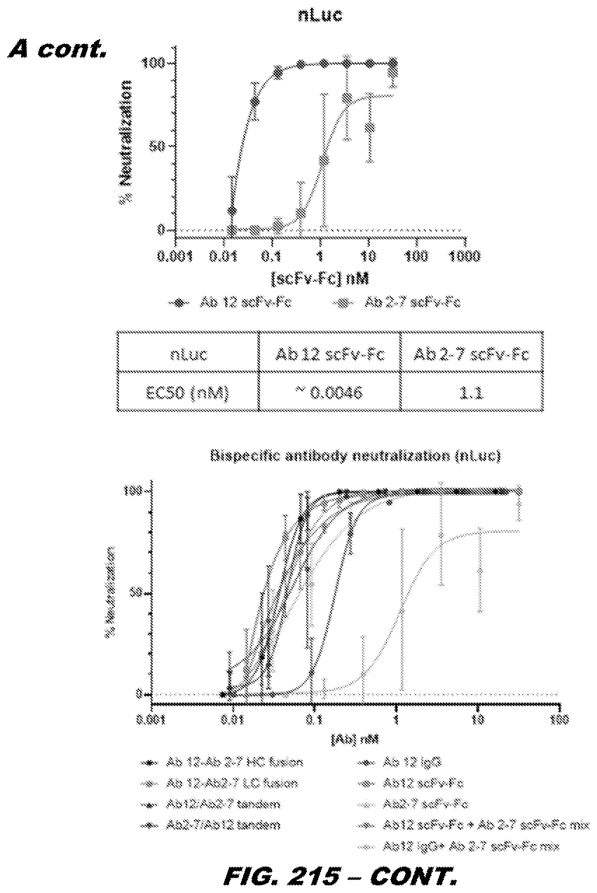

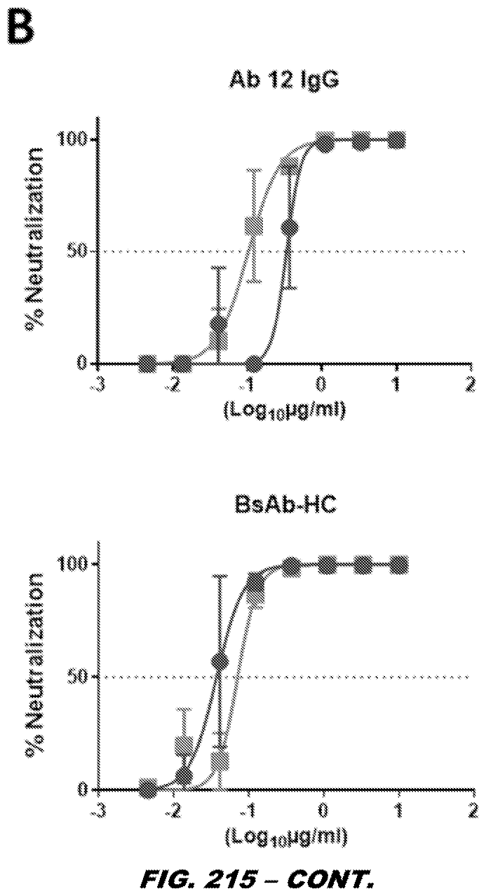

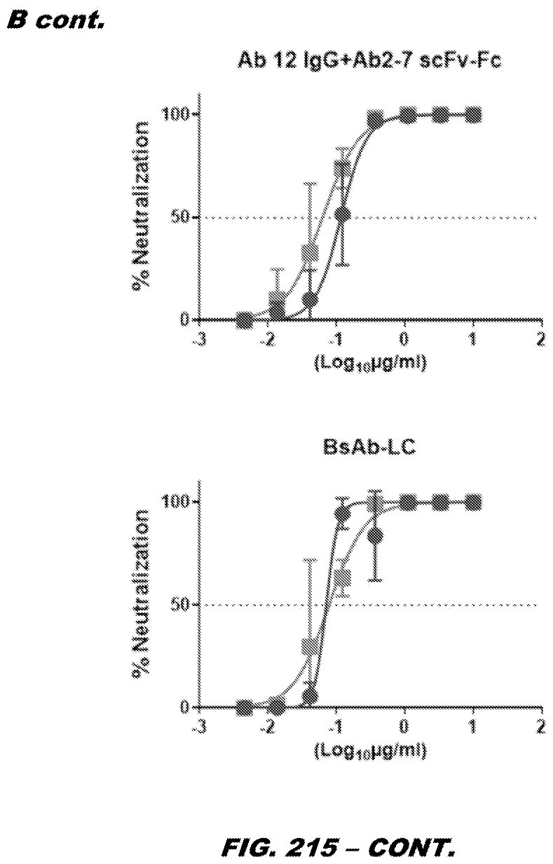

Studies were next initiated to assess dual binding of the BsAbs. Both the BsAb-HC (Ab 12 IgG-HC-Ab 2-7 scFv) and BsAb-LC (Ab 12 IgG-LC-Ab 2-7 scFv) fusion and tandem scFv-Fc (Ab 12 scFv/Ab 2-7 scFv-Fc and Ab 2-7 scFv/Ab 12 scFv-Fc) constructs were confirmed by BLI to bind both epitopes via competition assays (FIGS. 210-212). FACS studies on spike expressing cells showed quantitative differences in binding among these BsAbs (FIG. 213, FIG. 214). The 4 BsAbs can also capture soluble RBD, even when the Abs were already anchored to cell-surface spike, demonstrating that the binding arms are not occupied simultaneously (FIGS. 213A-B, FIG. 214). In addition, we replaced the Ab 2-7 scFv in the BsAb-HC and BsAb-LC with a control anti-PD1 scFv and observed a clear distinction between Ab 12 IgG-HC-αPD1 scFv and Ab 12-LC-αPD1 scFv where the former but not the latter was able to capture soluble RBD, demonstrating that only one arm of Ab 12 IgG-HC-αPD1 scFv was engaging its target epitope (FIGS. 213C-D, FIG. 215). Interestingly, some unexpected steric interferences were seen with control BsAb-HC fusions where substitution of Ab 12 or Ab 2-7 with an anti-PD1 IgG or scFv respectively allowed for blockade of both epitopes (FIGS. 210-212). In summary, both BsAb IgG-scFv fusions and tandem scFvs are able to achieve dual targeting and blockade.

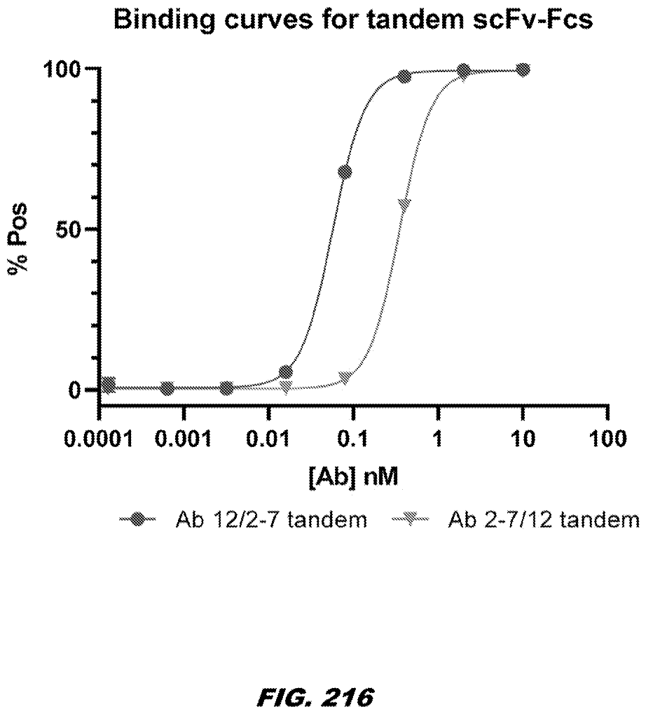

Synergistic effect of BsAbs on viral neutralization and mechanism of action. The BsAbs were tested in live virus neutralization experiments via PRNT and nLuc reporter viruses with comparable results (FIG. 216). The BsAbs were compared against the individual subcomponents or a mixture of parental Ab constructs at a 1:1 molar ratio, as this can contain equal number of Ab binding sites as the BsAbs. FIG. 203B shows that the BsAb-HC fusion had a 5- and 4.79-fold improvement in IC50 compared to Ab 12 IgG alone and the mixture respectively. Additionally, both the BsAb-LC fusion and Ab 12/2-7 tandem scFv-Fc achieved a 2-fold lower IC50 compared their respective mixtures. Ab 2-7/Ab 12 tandem scFv-Fc did not show any improvement (FIG. 203C) compared to the scFv-Fc cocktail.

We next performed synergy studies that emanated from cocktail studies demonstrating high levels of synergy between anti-SARS-CoV Abs CR3022 and CR3014(30). Using CompuSyn, dose response curves for the single and combination therapies were analyzed to calculate a combination index (CI) for synergy(35-37). Using this formula, CI values<1 or >1 indicate synergy and antagonism respectively, while a CI=1 signals an additive effect of the cocktail (36). As shown in FIG. 203D, both BsAb-HC and BsAb-LC fusions resulted in increased synergy compared to the IgG/scFv-Fc mixture, with the BsAb-HC reporting the highest levels (CI=0.12). The scFv-Fc mixture shows an additive effect while the different orientations of the tandem scFv-Fcs led to distinctly different outcomes (FIG. 203E). The Ab 12/2-7 tandem scFv-Fc increases synergy whereas Ab 2-7/12 tandem scFv-Fc shows an inhibitory effect with a CI>1, which is in agreement with the negative positional effect that we observed for binding in this orientation.

One mechanism of action for anti-spike nAbs is to induce premature shedding of the S1 domain as the post fusion spike is irreversibility disabled. Ab 12 containing constructs led to levels of S1 shedding comparable to ACE2, (FIG. 203F) demonstrating that Ab 12 binds in a way that simulates ACE2 binding, triggering the transition to the post fusion conformation. Similar to results seen by Wec et al. with CR3022, although Ab 2-7 locks the RBD in the “up” confirmation, it is unable to force spike shedding, demonstrating that neutralization is at least partially dependent on steric hinderance of ACE2 binding(26, 38).

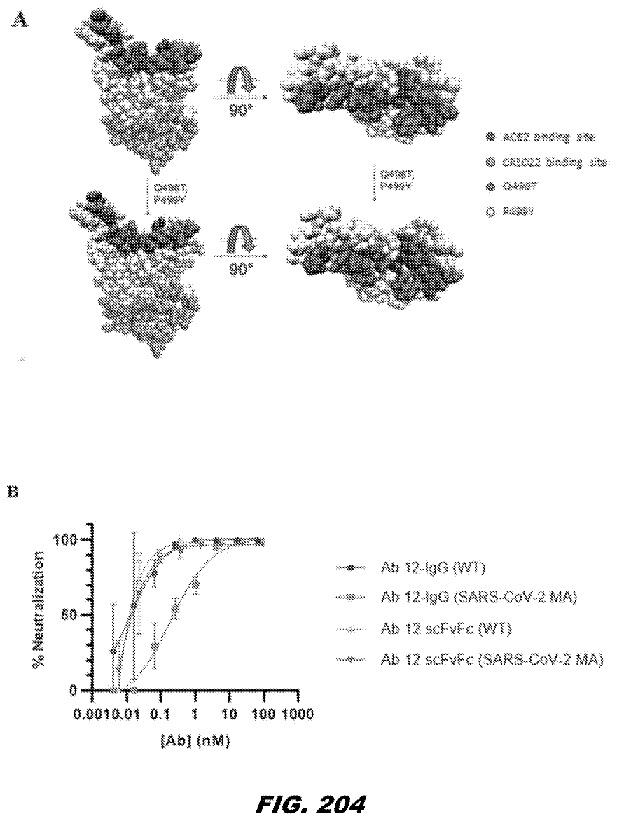

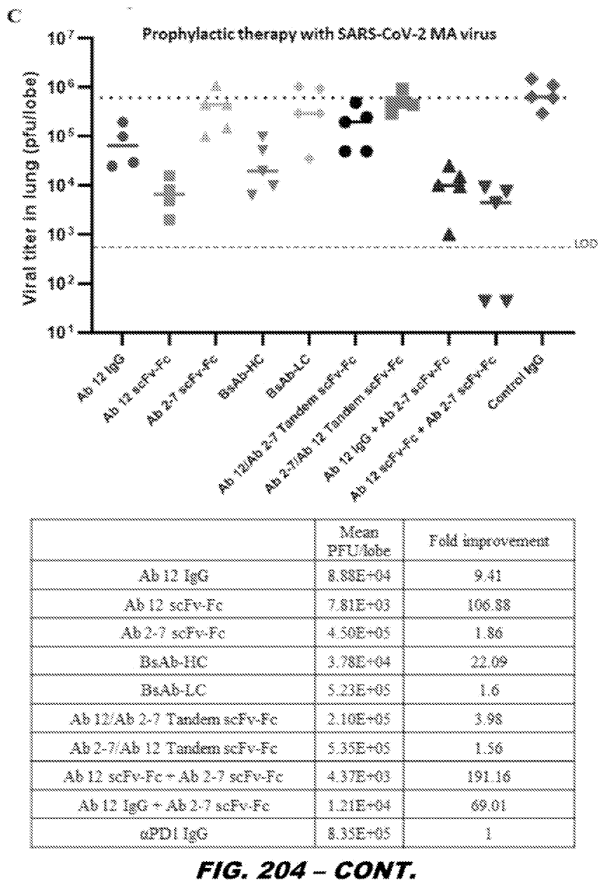

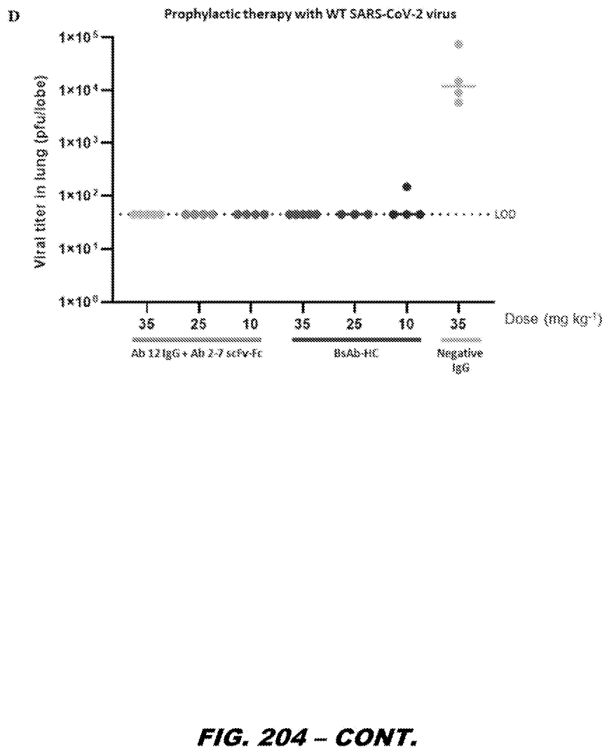

Prophylactic efficacy in vivo against SARS-CoV-2 virus. An aged Balb/c mice model using a mouse-adapted strain of SARS-CoV-2 virus (SARS-CoV-2 MA) was initially employed to assess in vivo protection(39). The adaptive mutations that allow the spike protein to recognize mACE2, Q498Y/P499T, reside in the RBD (FIG. 204A), creating a potential liability for RBD directed Abs. In vitro experiments were first performed to determine if our Abs were sensitive to these mutations. While Ab 12 scFv-Fc did not show any significant differences, the IgG showed a circa 10-fold decrease in neutralization efficacy towards the SARS-CoV-2 MA virus (FIG. 204B). Cryo-EM structures of Ab 12 Fab bound to spike (FIG. 201E) indicated that the Ab 12 epitope is near the Q498Y/P499T mutations, demonstrating that the difference in neutralization can result from changes to the direct binding epitope (FIG. 201G). Prophylactic administration of the mAbs and BsAbs demonstrated that the BsAb-HC fusion was the best performing BsAb in vivo, with a 22-fold reduction in virus titer while the BsAb-LC fusion and tandem scFv-Fcs had minimal efficacy against the SARS-CoV-2 MA strain (FIG. 204C). As for Ab cocktails, Ab 2-7 scFv potentiated the protective effect of Ab 12 IgG by 7-fold while the greatest prophylactic effect was seen with the scFv-Fc mixture.

The BsAb-HC and scFv-Fc mixture were selected for further testing in hACE2 transgenic mice to test their protection against WT virus(39, 40). We selected dosing concentrations to see complete neutralization while also comparing the effects of the SARS-CoV-2 MA virus in the previous experiment. FIG. 204D shows that both the scFv-Fc mixture and BsAb HC completely neutralized the virus at the Ab concentrations, with the exception of one animal at 10 mg kg-1 under BsAb-HC treatment that showed residual virus. Without wishing to be bound by theory, the circa 500-fold decrease in viral titers seen with the scFv-Fc mixture is comparable with what was seen in the SARS-CoV-2 MA experiment since the scFv-Fcs did not show differences for in vitro SARS-CoV-2 MA virus neutralization (FIG. 204B). The BsAb-HC fusion also achieved a circa 500-fold decrease at higher doses and approximately 350-fold reduction in titers at 10 mg kg-1 against the WT virus. This is a substantial improvement compared to its 22-fold reduction with the SARS-CoV-2 MA virus at the same concentration (FIG. 204C).

Neutralization escape. Neutralization escape studies were also performed with the individual parental antibodies, mixtures, and the BsAbs. We were unable to identify any escape mutants after 2 passages at 0.1 ug ml-1 followed by 3 passages at 1 ug ml-1 for mixtures containing Ab 12. As Ab 2-7 is less potent, the study was performed at 10× the concentration of Ab 12 constructs at each step with similar results.

### DISCUSSION

Since the sequence of the SARS-CoV-2 spike protein was first published in early January 2020, a large number of nAbs have been identified from convalescent patient B-cells, animal immunization, and phage display. In this work similar to studies that were performed on SARS-CoV and MERS-CoV(41, 42), we utilized our naïve phage library to identify two nAbs that target divergent epitopes on the RBD. Ab 12 targets the ACE2 binding domain, is a potent neutralizer and induces spike shedding, while Ab 2-7 exhibits maximal neutralization at higher concentrations, targets the conserved region targeted by anti-SARS-CoV Ab CR3022 and sterically blocks ACE2. Conversion of Ab 2-7 scFv to Fab resulted in a significant and unexpected decrease in neutralization ability without any significant change in its affinity to recombinant RBD. BsAbs were designed that utilized both of these Abs, resulting in synergistic in vitro neutralization and potent in vivo protective activity over the parental monospecific Abs.

Structural analysis of the Ab 2-7 scFv bound to stabilized spike clearly shows that bound scFv locks the RBD in the “up” position, similar to that found by CR3022 binding(26). Ab 2-7 binds an overlapping but distinct epitope from CR3022 as Ab 2-7 infringes upon the space required for ACE2 binding and it is possible that neutralization solely results from this blockade. However, the demonstration by Huo et al. that CR3022 allosterically perturbs RBD-ACE2 binding and destabilizes the spike, provides another potential mechanism of action for Ab 2-7 neutralization that requires further investigation(38, 43). We observed steric constraints on conversion of Ab 2-7 scFv into a Fab which implies that the scFv-phage display platform provided a unique mode of discovery for Ab 2-7 as the epitope is shielded from BCR recognition by the same steric constraints. These steric constraints also play a similar role in the Ab 2-7 scFv-Fc format that resulted in binding of one arm at a time.

Other groups have previously developed bi- and tri-specific VHH (nanobody) based constructs and these were made by sequentially linking VHHs targeting the ACE2 binding domain of the RBD(44-46). As nanobodies are monovalent, the fusion of multiple VHHs in parallel is important for increasing valency and avidity, providing a marked increase in neutralization for many of these constructs. The BsAbs described herein are built by combining Ab 12 and Ab 2-7 in various Ab formats (IgG, scFv-Fc, scFv), and are inherently symmetric and multi-paratopic. Instead of focusing strictly on the ACE2-RBD interface, the BsAbs are targeted to both the ACE2-binding interface and the conserved, non-ACE2 binding domain of the RBD, providing multiple mechanisms of action for viral neutralization. Synergy analysis using the median-effect equation showed that the heavy and light chain fusions both displayed substantial improvements in synergy compared to the parental Ab mixture. This is in line with our in vitro data as the BsAb-HC fusion has the highest levels of synergy and the greatest levels of neutralization against WT virus in vitro.

In vitro neutralization studies showed no difference in the ability of mono- and BsAbs to neutralize D614G SARS-CoV-2 virus. Results unexpectedly showed that Ab 12 IgG exhibited a >10-fold decrease in neutralization efficacy against the SARS-CoV-2 MA strain whereas we did not see this neutralization difference with Ab 12 scFv-Fc. The Q498Y/P499T adaptive mutations in the SARS-CoV-2 MA strain reside proximal to the Ab 12 RBD epitope and provide an example of mutations that can lead to the development of Ab resistance. In addition, while the BsAb-LC fusion and tandem scFv-Fc constructs had very strong performance in vitro, they had minimal efficacy in halting SARS-CoV-2 MA replication in a prophylactic aged Balb/c mouse model. The BsAb-HC fusion was the best BsAb, reducing viral burden by >20-fold compared to control treated animals. Based on these results, and the increased level of synergy observed, we chose the BsAb-HC and scFv-Fc mixture for expanded testing against WT virus in transgenic hACE2 mice. Prophylactic treatment with the BsAb-HC fusion and scFv-Fc cocktail led to profound neutralization of WT virus in the lung and overall showed better performance than with the mouse-adapted SARS-CoV-2 strain. An important consideration in comparing the BsAb-HC fusion and scFv-Fc cocktail is the relative size of each construct and the amount of protein dosed. Where there are equal number of antigen binding sites in the cocktail, the mass of the BsAb-HC fusion is ˜2× greater than that of an scFv-Fc (203 kDa vs 105 kDa), resulting in a molar concentration circa half that of the scFv-Fc fusion.

The successful development of human BsAbs against SARS-COV-2 is an engineering advance in the field. We have identified two unique human nAbs and demonstrated that synergistic neutralization activity can be achieved by targeting divergent non-overlapping epitopes on the SARS-CoV-2 RBD. A promising step in their clinical development is that binding to FcRn remains intact so that Ig recycling and catabolism cannot be compromised (FIG. 209E). Innovative strategies are urgently needed to optimize nAb potency and durability as well as accelerate their delivery and affordability so that access to everyone, including those who live in resource poor regions of the globe can be achieved. Further in vitro and in vivo studies are still needed to elucidate a lead candidate for therapeutic development, but the studies here offer a fresh path toward the development of next generation anti-SARS-CoV-2 BsAb drugs.

### Materials and Methods

Phage panning. Peripheral B cells from 57 healthy donors were used to create two, non-immunized scFv-phage libraries totaling 2.7×10¹⁰ members. 1.66×10¹² pfu of scFv-phage from each library was combined and used to perform 3 rounds of panning against SARS-CoV-2 S1 protein (Sino Biologicals) or SARS-CoV-2 RBD protein expressed. Briefly, SARS-CoV-2 RBD or S1 proteins were passively absorbed onto Nunc MaxiSorp Immuno tubes (Thermo Fisher Scientific) overnight in PBS. Coated tubes were incubated with the phage library, followed by PBS/PBS-T (PBS+0.05% Tween-20) washes to remove nonspecific phage. Bound phage were eluted with 100 mM triethylamine and neutralized with 1 M Tris-HCl, pH 7.5. The eluted phage solution was neutralized, amplified, and used for further selection or screening. SARS-CoV-2 S1 and RBD coating concentration was decreased in each round to increase the affinity of the enriched antibodies.

Screening of the enriched library was performed by selecting circa 1300 bacterial colonies from the 3rd round of panning and culturing them in individual wells in 96 well plates. Small scale rescue was performed via VCS-M13 helper phage and the phage supernatant was used to screen via SARS-CoV-2 RBD or S1 coated ELISA plates. Positive wells were selected for colony PCR and subsequent sanger sequencing. Unique sequences were then cloned into the appropriate expression vector for further analysis.

BsAb design. BsAbs were designed to utilize different functional formats of Abs 12 and 2-7. IgG fusions were built using Ab 12 IgG as the scaffold, with Ab 2-7 scFv genetically fused to the C terminus of the CL (BsAb-LC fusion) or CH3 (BsAb-HC fusion) domains via a flexible (G4S)5 or (G4S)2 linker respectively. Tandem scFv-Fc construct consists of two scFvs linked with a flexible (G4S)3 linker fused to the IgG1 hinge-Fc domains and was created in both orientations (Ab 12/Ab 2-7 and Ab 2-7/Ab 12)

Recombinant SARS-CoV-2 protein production. hACE2 (transOMIC) and SARS-CoV-2 RBD/S1 (Sino Biologics) cDNA was purchased and cloned into our mammalian expression vector. Stabilized SARS-CoV-2 spike trimer expressing plasmid was obtained through BEI and the HexaPro expression plasmid was a kind gift from Dr. Jason McLellan's Lab (UT Austin). Proteins were expressed in the Expi293F system and cells were transiently transfected by Expifectamine 293 (ThermoFisher) following the standard protocol. 4-5 days after transfection, supernatants were clarified and incubated with Ni-NTA resin (Qiagen) overnight at 4° C. They were subsequently purified via gravity flow column and buffer exchanged by centrifugation in Amicon centrifugal filters. Avi tagged proteins were biotinylated by Avidity's BirA biotiniylation kit following standard protocols. Protein concentration was measured on a Nanodrop 100 using the MW and extinction coefficients calculated on ExPASy's ProtParam.

Antibody production. Recombinant scFv-Fc, IgG, and bispecific antibodies were produced in Expi293F cells (ThermoFisher Scientific). Mammalian expression vectors encoding the antibodies were transiently transfected using Expifectamine 293 following the standard protocol and cultivated for four days. The harvested supernatants were incubated with Protein A-Sepharose 4B resin (Invitrogen) overnight at 4° C. followed by purification via gravity flow columns (BioRad) and buffer exchanged by centrifugation in Amicon centrifugal filters. Protein concentration was measured on a Nanodrop 100 using the MW and extinction coefficients calculated on ExPASy's ProtParam.

Biolayer interferometry (BLI) binding assays. Assays were performed in 96-well black plates on an Octet Red96 instrument (ForteBio) with shaking at 1,000 RPM. Sensors were loaded with the analyte of interest, followed by association of the appropriate sample. Samples were made in PBST (PBS+0.5% Tween-20, Boston Bio Products) except for FcRn binding assay, which was prepared in PBS titrated to pH 6 with hydrochloric acid. Curve fitting was performed using a 1:1 binding model in the Octet data analysis software. Mean K_{D}, kₒₙ, k_{off} values were determined with a global fit.

Pseudovirus assays. Full length SARS-CoV-2 spike was cloned into a mammalian expression vector with a gp41 tail to improve pseudovirus integration. The day before transfection, LentiX-293T cells (Takara) were seeded in 150 mm dishes in DMEM+10% FBS+pen/strep and cultured in a humidified incubator at 37° C., 5% CO₂. On the day of transfection, spike expressing plasmid was mixed with pseudovirus packing and luciferase reporter plasmids and transfected via polyethylenimine MAX (Polysciences). 48 hours after transfection, the supernatant was collected and the cellular debris was removed via centrifugation. Immediately before use, the clarified supernatant was filtered via 0.45 SFCA syringe filter.

293T-ACE2 cells were obtained through BEI and cultured in DMEM+10% FBS+pen/strep. Passage number was maintained below 15. 10,000 ACE2+ cells were seeded the day before the assay in 100 ul of complete DMEM. For pseudovirus neutralization assays, antibodies were diluted in complete DMEM and mixed 1:1 with virus (30 ul+30 ul) and incubated at RT for 60 min, followed by 10 min at 37° C. to warm the media before adding to cells. Culture supernatant was removed from the cells and carefully replaced with 60 ul of virus/Ab mixture. The plates were incubated at 37° C. for 48 hours, before the supernatant was removed and cells were lysed with Promega's passive lysis buffer. Pseudovirus infection was measured by luciferase activity in the cell lysate and was detected using Promega's BioGlo luciferase reagent. Samples were read on a Polarstar Omega plate reader.

Purification of Ab 2-7 for cryo-electron microscopy. HEK293F cells were grown in suspension and transfected with pcDNA3.1-Ab 2-7 scFv-6×His using polyethylenimine. The culture was maintained at 37° C. and 8% CO₂ for six days. The medium was harvested by centrifugation, then concentrated and buffer exchanged into phosphate buffered saline. Ab 2-7 scFv was purified from the resulting solution by affinity chromatography and size exclusion chromatography. Concentrated medium was passed over 5 mL of Nickel-NTA resin, washed with 1×PBS supplemented with 40 mM imidazole, and eluted with 1×PBS supplemented with 250 mM imidazole. Elution fractions containing the Ab2-7 were dialyzed into 1×PBS to remove imidazole, concentrated in a centrifugal filter (10,000 kDa cutoff), and injected onto a Superdex 200 size exclusion column (GE Healthcare). The peak corresponding to purified, monomeric Ab 2-7 scFv was concentrated in a centrifugal filter (10,000 kDa cutoff). Concentrated aliquots (19 mg/mL) were flash frozen in liquid nitrogen and stored at −70° C. for further use.

Cryo-electron microscopy of the complex between SARS-CoV-2 S-D614G and Ab 2-7 scFv. Prefusion-stabilized SARS-CoV-2 S-2P-D614G (ExcellGene SA) was concentrated to 1 mg/mL and incubated with a 5-fold molar excess of Ab 2-7 scFv. Samples were deposited onto plasma-cleaned gold grids (UltrAuFoil R 1.2/1.3, Au 300), plunge frozen in liquid ethane, and loaded into a Talos F200C transmission electron microscope equipped with a Ceta 16M detector (ThermoFisher Scientific). Grids were imaged at a magnification of 92,000×, corresponding to a pixel size of 1.63 Å. Contrast transfer function estimation and particle picking were performed in cisTEM (47). Extracted particles were exported to cryoSPARC-v2 (Structura Biotechnology Inc.) for 2D classification, ab initio 3D reconstruction and refinement. C1 symmetry was used during homogeneous refinement. Models were docked into the experimental EM density using Chimera and Phenix. Two starting models were used: SARS-CoV-2 S with two RBDs in the “up” conformation (PDB ID 7K8Y), and a homology model of Ab 2-7 scFv that was generated using the SAbPred server (48,49).

Cryo-electron microscopy of the complex between SARS-CoV-2 S-D614G and Ab 12 Fab. 0.5 mg of Prefusion-stabilized SARS-CoV-2-D614G was incubated with a 10-fold molar excess of Ab12 Fab overnight. The complex was purified by size exclusion chromatography on a Superose 6i 10/300 GL column and concentrated to 2 mg/ml. Electron microscopy grids were prepared by placing a 3 ul aliquot of the sample on a plasma-cleaned C-flat grid (2/1C-3T, Protochips Inc) and immersing it in liquid ethane for vitrification. The grid was then loaded into a Titan Krios G3 electron microscope (ThermoFisher Scientific) equipped with a K3 direct electron detector (Gatan Inc) at the end of a BioQuantum energy filter, using an energy slit of 20 eV. The microscope was operated with an accelerating voltage of 300 kV. Grids were imaged at a magnification of 75kX, corresponding to a pixel size of 0.66 Å. Motion correction, CTF estimation, and particle-picking were done with Warp. Extracted particles were exported to cryoSPARC-v2 (Structura Biotechnology Inc.) for 2D classification, ab initio 3D reconstruction, and refinement. C1 symmetry was used during homogeneous refinement. Models were docked into the experimental EM density using Chimera and Phenix. One starting model was used: SARS-CoV-2 S with two RBDs in the “up” conformation (PDB ID 7K8T), and a homology model of Ab12 Fab that was generated using the SAbPred server

Plaque reduction neutralization test. A series of 10 half-log dilutions was prepared in triplicate for each antibody or antibody mixture in Dulbecco's Phosphate Buffered Saline (DPBS) (Gibco). Each dilution was incubated at 37° C. and 5% CO₂ for 1 hour with an equal volume of 1000 plaque forming units/ml (PFU/ml) of SARS-CoV-2 (isolate USA-WA1/2020), diluted in Dulbecco's Modified Eagle Medium (DMEM) (Gibco) containing 2% fetal bovine serum (Gibco) and antibiotic-antimycotic (Gibco). Controls included DMEM containing 2% fetal bovine serum and antibiotic-antimycotic only as a negative control, 1000 PFU/ml SARS-CoV-2 incubated with DPBS, and 1000 PFU/ml SARS-CoV-2 incubated with DMEM. Two hundred microliters of each dilution or control were added to confluent monolayers of Vero E6 cells in triplicate and incubated for 1 hour at 37° C. and 5% CO₂. The plates were gently rocked every 5-10 minutes to prevent monolayer drying. The monolayers were then overlaid with a 1:1 mixture of 2.5% Avicel® RC-591 microcrystalline cellulose and carboxymethylcellulose sodium (DuPont Nutrition & Biosciences, Wilmington, DE) and 2× Modified Eagle Medium (Temin's modification, Gibco) supplemented with 2× antibiotic-antimycotic (Gibco), 2× GlutaMAX (Gibco) and 10% fetal bovine serum (Gibco). Plates were incubated at 37° C. and 5% CO₂ for 2 days. The monolayers were fixed with 10% neutral buffered formalin and stained with 0.2% aqueous Gentian Violet (RICCA Chemicals, Arlington, TX) in 10% neutral buffered formalin for 30 min, followed by rinsing and plaque counting. The half maximal inhibitory concentrations (IC50) were calculated using GraphPad Prism 8

nLuc virus. Vero E6 cells were plated at 20,000 cells per well in black-walled 96-well plates (Corning 3904). mAbs were serially diluted 3-fold with a maximum of eight dilution spots. Diluted antibodies were mixed with 85 PFU/well of recombinant SARS-CoV-2-nLuc virus, and the mixtures were incubated at 37° C. with 5% CO₂ for 1 hour. Following incubation, growth media was removed, and virus-antibody mixtures were added to the cells in duplicate. Virus-only controls were included in each plate. Following infection, plates were incubated at 37° C. with 5% CO₂ for 48 h. After the 48 h incubation, cells were lysed, and luciferase activity was measured via Nano-Glo Luciferase Assay System (Promega) according to the manufacturer specifications. Neutralization titers were defined as the sample dilution at which a 50% reduction in relatively light unit (RLU) was observed relative to the average of the virus control wells.

Serum in vitro neutralization assay. Aliquots of mNeonGreen reporter SARS-CoV-2 were pre-incubated for 1 h in 5% CO₂ at 37° C. with serial 4-fold dilutions of serum and inoculated into Vero-E6 triplicate monolayers in black polystyrene 96-well plates with clear bottoms (Corning). The final amount of virus was 200 PFU/well, serum was diluted with an initial 1:20 dilution followed by 2×fold dilutions. Cells were maintained in Minimal Essential Medium (ThermoFisher) supplemented by 10% FBS (HyClone) and 0.1% genamicin in 5% CO₂ at 37° C. After 2 days of incubation, fluorescence intensity of infected cells was measured at a 488 nm wavelength using a Cytation 5 Cell Imaging Multi-Mode Reader (Biotek). The signal readout was normalized to virus control aliquots with no serum added and was presented as the percentage of neutralization.

FACS binding. 293T cells were transduced to stably express SARS-CoV-2 spike protein. 2E5 cells were washed and resuspended in cold MACS rinsing buffer+BSA (Miltenyi) before adding Abs diluted in cold MACS buffer. Cells were incubated at 4° C. for 1 hour to allow for antibody binding, after which they were washed 2× with MACS buffer before incubation with fluorescently labeled anti-hFc (BioLegend) for 20 min at 4° C. Cells were washed 3× with cold MACS buffer before being fixed with 1% paraformaldehyde. Cells were analyzed on a BD Canto II with HTS reader. Samples were run in triplicate.

FACS S1 disassociation. 293T-Spike cells were washed, resuspended at 4E6 cells/ml in MACS buffer with 20 uM cycloheximide (MACS+) to inhibit protein synthesis, and aliquoted at 50 ul per well in a V bottom 96 well plate (50). Abs were diluted to 200 nM in MACS+ and both Ab dilution and cell plates were incubated separately at 37° C. for 15 min to equilibrate the plates. At the time points described herein, 50 ul of Ab dilution was transferred to the corresponding well in the 96 well plate and mixed via pipetting. The plate was maintained at 37° C. during the entire time course. After the last timepoint, the cell plates were rapidly transferred to ice and quenched with ice cold MACS buffer. The plate was washed 2× with MACS buffer, followed by resuspension in anti-hFc-APC (BioLegend) for 20 min at 4° C. Cells were washed 3× with MACS buffer before fixation by 1% paraformaldehyde (Boston Bio Products). Cells were analyzed on a BD Canto II with HTS reader. Samples were run in duplicate.

FACS dual binding. 2E5 293T-Spike cells were incubated with specified antibodies at various concentrations for 1 hour at 4° C. After washing, cells were incubated with a fixed amount of RBD-biotin (expressed in house) at RT or 4° C. for one 1 hour. Cells were then washed and incubated with anti-hFc-PE and streptavidin-APC (BioLegend) for 25 min at 4° C. After thorough washing, cells were fixed with 1% paraformaldehyde and analyzed on a BD Canto II with HTS reader.

Syrian golden hamster experiments. Syrian hamster SARS CoV virus challenge study. Animal challenge studies were conducted in the ABSL-4 facility. The animal protocol for testing of mAbs in mice was approved by the Institutional Animal Care and Use Committee (IACUC).

1 day before the challenge, hamsters were microchipped. On day 0, hamsters were anesthetized with ketamine/xylazine and challenged with SARS-CoV-2 by the intranasal (IN) route with up to 10\hat{ }7 TCID50 (or 10\hat{ }6 PFU/ml) in a total volume up to 100 μL. The viral strain used is Wuhan Hu-1 strain, SARS-CoV strain 2019-nCoV/USA_WA1/2020 (WA1); GenBank: MN985325; GISAID: EPI_ISL_404895. For animal experiments passage 5 was used. The final challenge dose was 10000 plaque forming units diluted in sterile PBS. Body weight and body temperature were measured each day, starting at day 0.

On day 1 post challenge (dpc) hamsters were treated with 5 mg/kg of monoclonal antibodies diluted in 0.5 ml of sterile PBS via intraperitoneal route (IP). The control group received an equal volume of sterile PBS via the same IP route. On day 3 post challenge the animals were sacrificed. At necropsy, terminal blood was collected into a labeled 3.5 mL SST vacutainer from the animals. Lungs were harvested for the groups.

Syrian golden hamster tissue processing and viral load determination. For the pathogenicity study, animals from each study group were euthanized on day 3 post challenge, and the lungs were harvested. Right lungs were placed in L15 medium supplemented with 10% fetal bovine serum (Gibco) and Antibiotic-Antimycotic solution (Gibco), flash-frozen in dry ice and stored at −80° C. until processing. Tissues were thawed and homogenized using the TissueLyser II system (Qiagen). Tissue homogenates were titrated on Vero E6 cell monolayers in 96-well plates to determine viral loads. 10× fold dilutions of the lung supernatants were incubated for 1 hour and replaced with 100 μLs of 0.9% methylcellulose in minimal essential medium (MEM) containing 10% fetal bovine serum (Quality Biologicals) and 0.1% gentamicin sulfate (Mediatech), followed by incubation at 37° C. Plates were fixed with 10% buffered formalin (Thermo Fisher) with subsequent removal from the biocontainment laboratories. Foci were visualized by staining monolayers with a mixture of 37 SARS-CoV-2 specific human antibodies kindly provided by Distributed Bio. As the secondary antibody, HRP-labeled goat anti-human IgG (SeraCare) was used at dilution 1:500. Primary and secondary antibodies were diluted in 1×DPBS with 5% milk. Plaques were revealed by AEC substrate (enQuire Bioreagents).

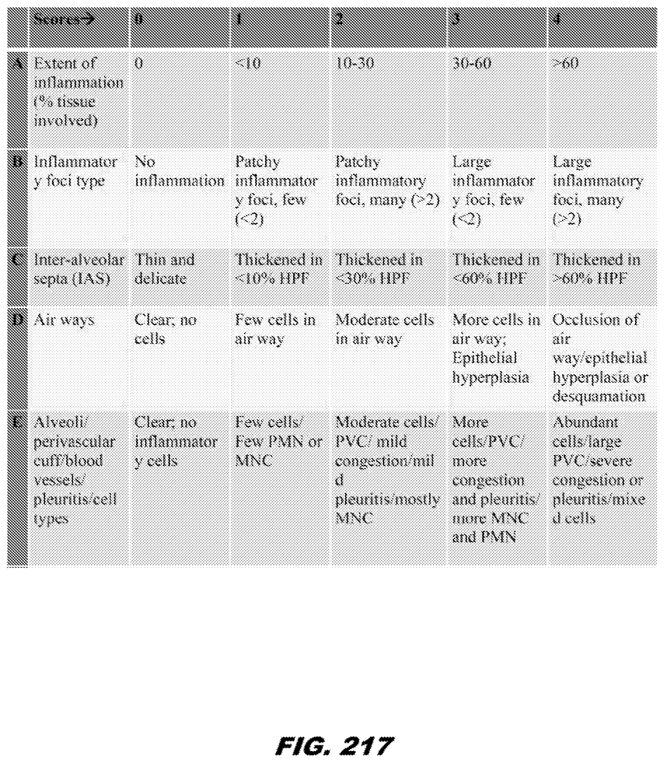

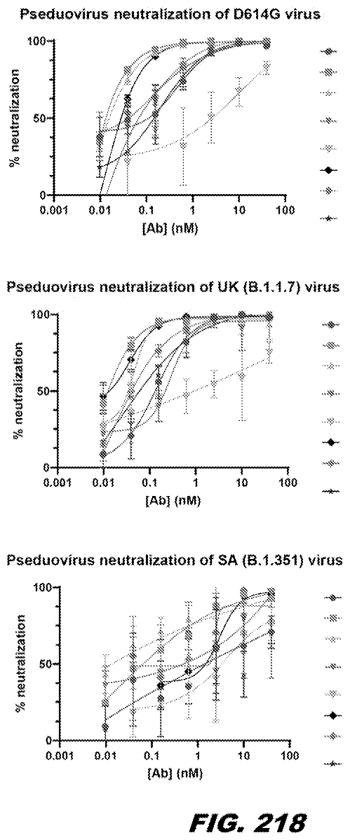

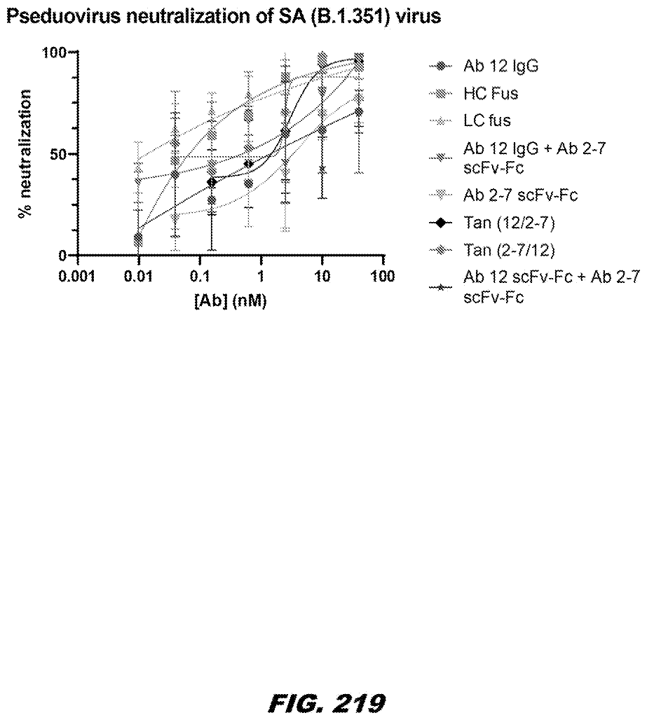

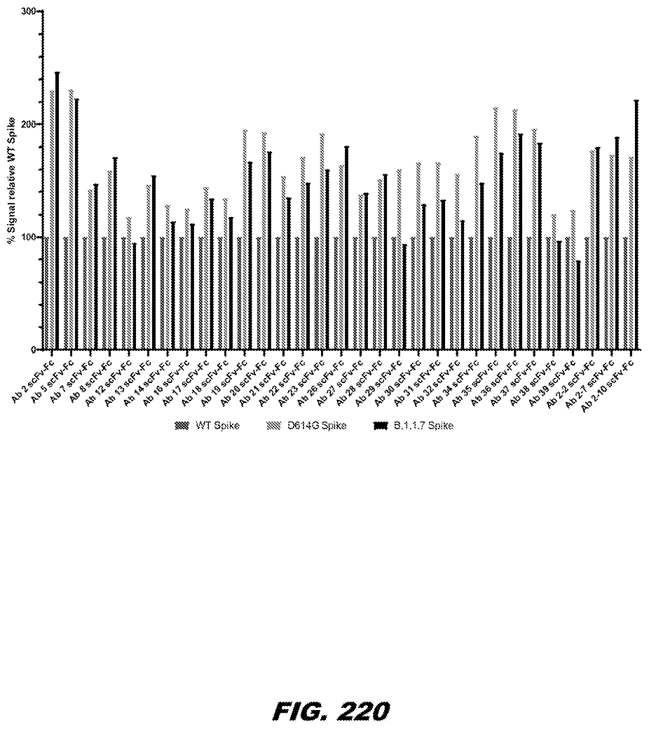

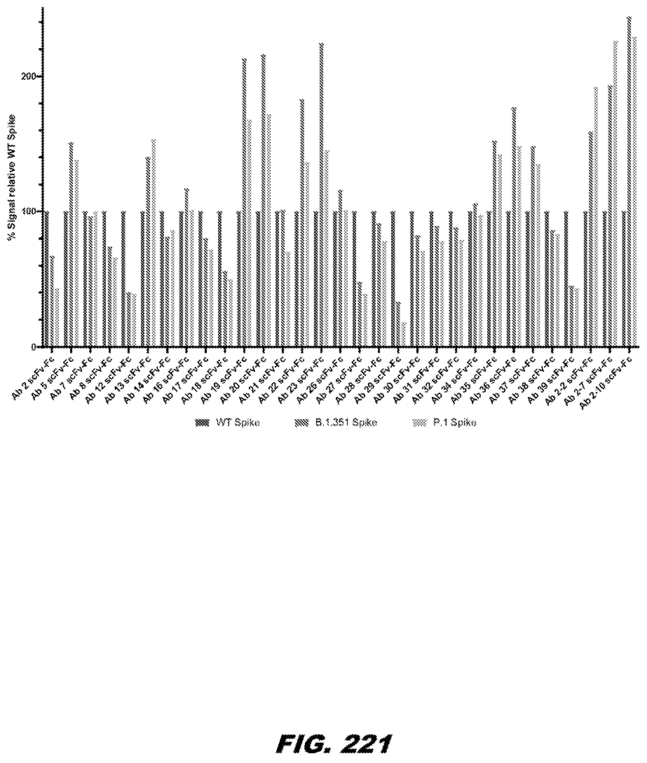

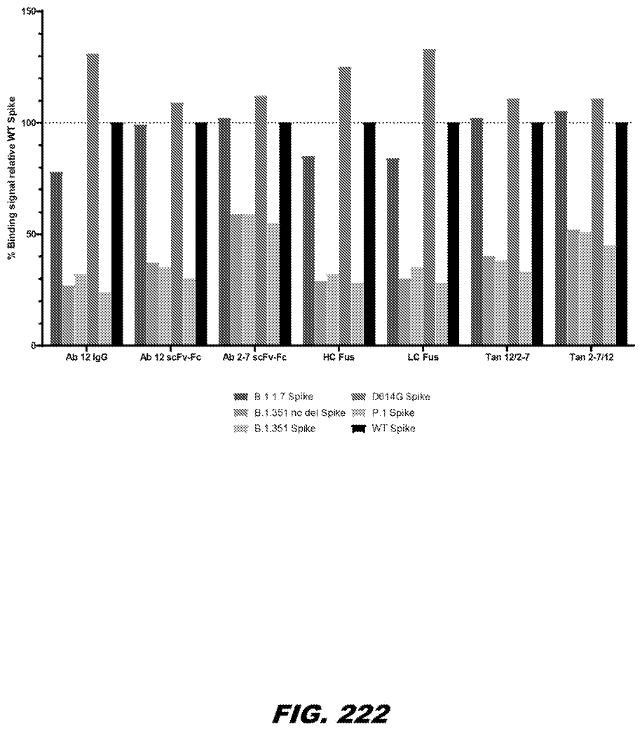

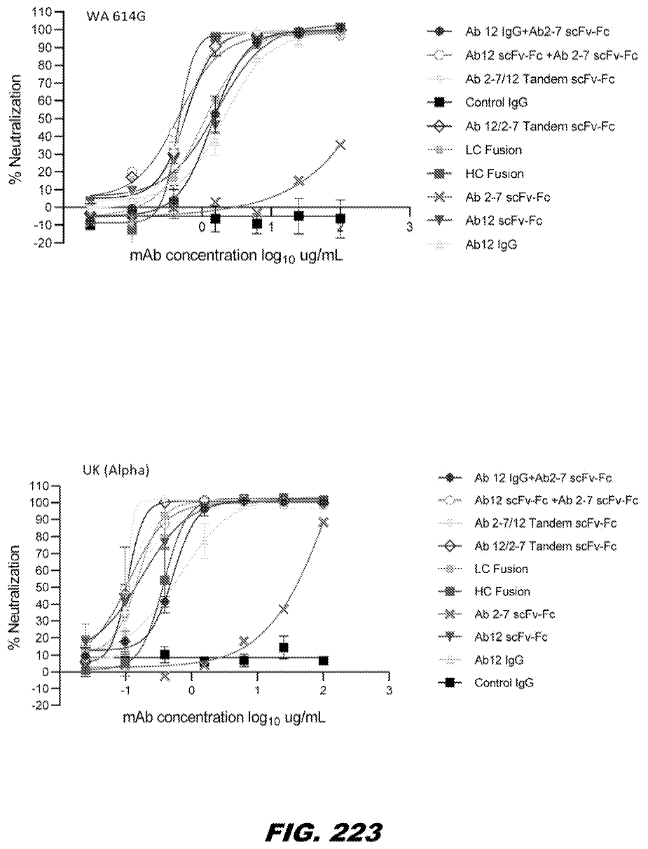

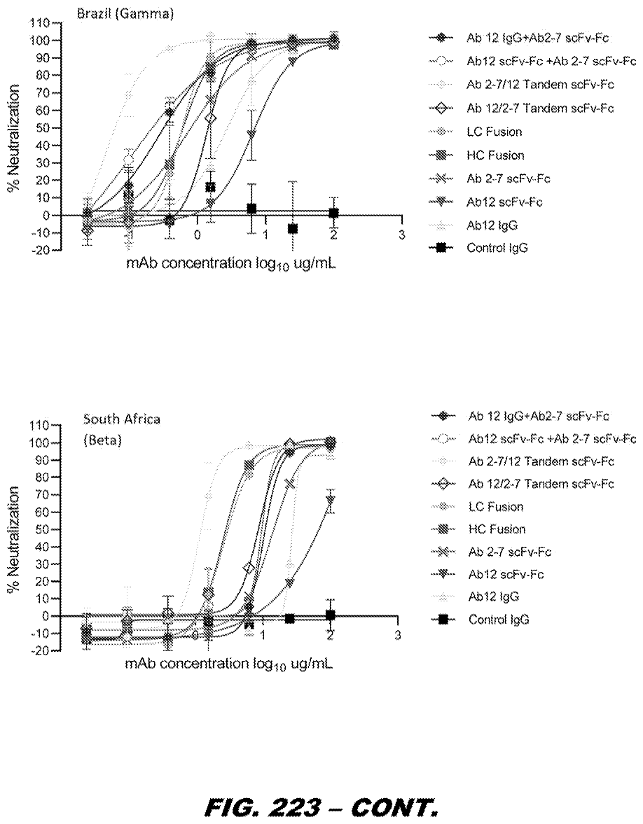

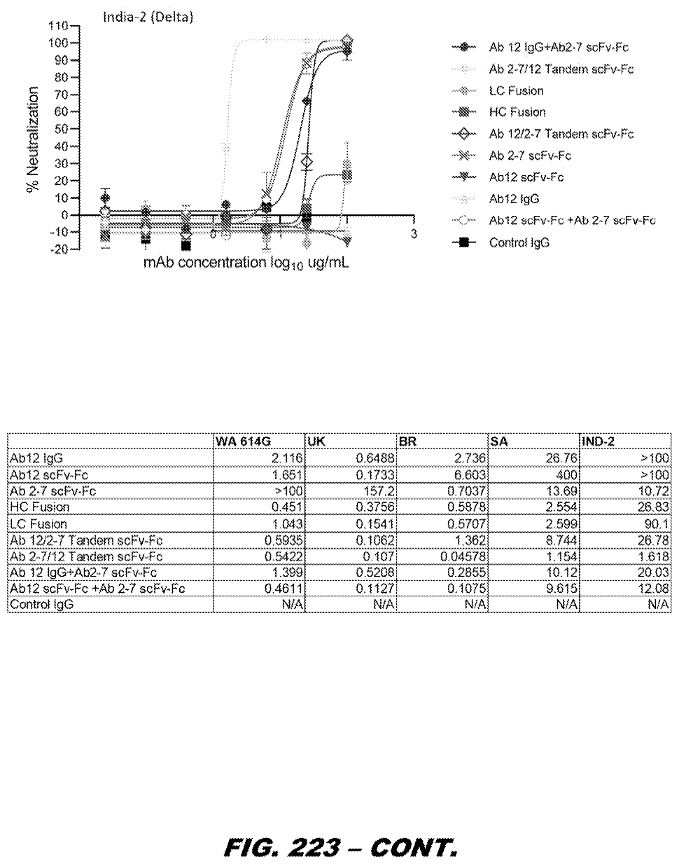

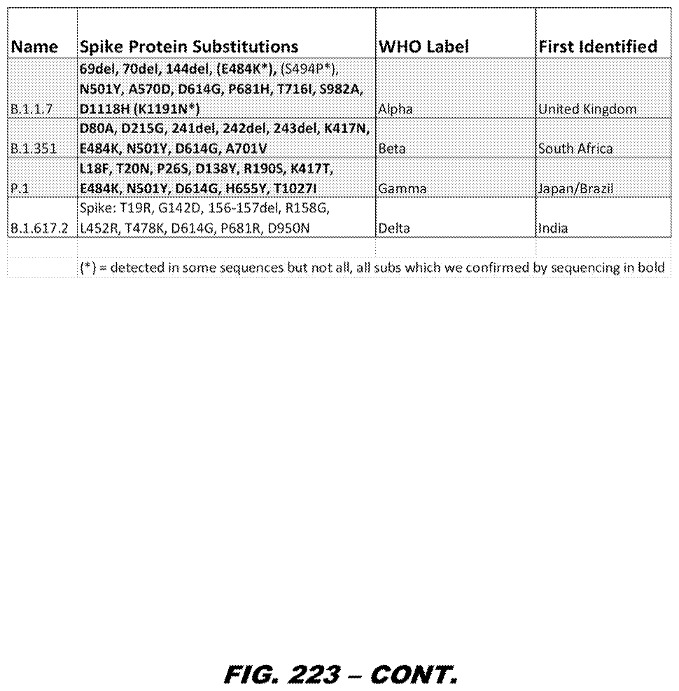

Syrian golden hamster histopathology. During necropsy, gross lesions were noted and representative lung tissues from the left lobe were collected in 10% formalin. After a 24-hour initial fixation at 4° C., the lung tissues were transferred to fresh 10% formalin for an additional 48-hour fixation, prior to removal from containment. Formalin-fixed tissues were processed by standard histological procedures. About 4 μm-thick sections were cut and stained with hematoxylin and eosin (HE). Sections of lungs were examined for the extent of inflammation, type of inflammatory foci, and changes in alveoli/alveolar septa/airways/blood vessels in parallel with sections from uninfected or control animals. The blinded tissue sections were semi-quantitatively scored for pathological lesions using the criteria described in FIG. 217. Slides were scored by a trained staff member. Significance was assessed using a Kruskall-Wallis test with Dunn's post-hoc correction.

Prophylactic efficacy in mouse models. Eleven to twelve-month old female BALB/c mice (BALB/c AnNHsd, Envigo, stock #047) were used for mouse-adapted SARS-CoV-2 (SARS-CoV-2 MA) in vivo protection experiments as described previously (39). Ten-week-old HFH4-hACE2 transgenic mice were used for SARS-CoV-2 WT in vivo protection experiments (39,40). For evaluating the prophylactic efficacy of single mAbs and mAb combinations, mice were injected intraperitoneally (ip) with the appropriate concentration of each mAb combination 12 hours prior to infection. Mice were infected intranasally with 1×10⁵ PFU SARS-CoV-2 MA or SARS-CoV-2 WT, respectively. At 48 hours post infection, mice were sacrificed, and lung tissue was harvested for viral titer as measured by plaque assays. For viral plaque assays, the caudal lobe of the right lung was homogenized in PBS, and the tissue homogenate was then serial-diluted onto confluent monolayers of Vero E6 cells, followed by agarose overlay. Plaques were visualized with overlay of Neutral Red dye on day 2 post infection. Mouse studies were performed using protocols approved by Institutional Animal Care and Use Committee (IACUC) and were performed in a BSL3 facility.

Generation of viral escape mutants. A549-ACE2 cells were infected with SARS-CoV-2-nLuc virus at MOI of 0.1 for 1 hour and maintained in medium containing mAbs. Viral supernatants were passaged five times in naïve A549-ACE2 cells with 3 day intervals. For the 1st and 2nd passages, 1 μg ml-1 of Ab 2-7 scFv-Fc and 0.1 μg ml-1 of the rest of mAbs were added to the cell lines. For the 3rd to 5th passages, the concentrations of mAbs were increased 10-fold. Viral titers were determined by plaque assay and the S gene sequences were determined by Sanger sequencing.

### REFERENCES FOR THIS EXAMPLE

1. COVID-19 Dashboard by the Center for Systems Science and Engineering (CSSE) at Johns Hopkins University (JHU) (2020).
2. World Bank, “Global Economic Prospects, June 2020” (2020) https:/doi.org/10.1596/978-1-4648-1553-9.
3. Z. Lv, et al., Structural basis for neutralization of SARS-CoV-2 and SARS-CoV by a potent therapeutic antibody. Science 369, 1505-1509 (2020).
4. D. Pinto, et al., Cross-neutralization of SARS-CoV-2 by a human monoclonal SARS-CoV antibody. Nature 583, 290-295 (2020).
5. T. F. Rogers, et al., Isolation of potent SARS-CoV-2 neutralizing antibodies and protection from disease in a small animal model. Science 369, 956-963 (2020).
6. B. Ju, et al., Human neutralizing antibodies elicited by SARS-CoV-2 infection. Nature (2020) https:/doi.org/10.1101/2020.03.21.990770.
7. D. Wrapp, et al., Cryo-EM structure of the 2019-nCoV spike in the prefusion conformation. Science 367, 1260-1263 (2020).
8. X. Fan, D. Cao, L. Kong, X. Zhang, Cryo-EM analysis of the post-fusion structure of the SARS-CoV spike glycoprotein. Nat. Commun. 11, 1-10 (2020).
9. R. Yan, et al., Structural basis for the recognition of the SARS-CoV-2 by full-length human ACE2. Science 2, 1444-1448 (2020).
10. Regeneron Pharmaceuticals, “Regeneron's COVID-19 Outpatient Trial Prospectively Demonstrates that REGN-COV2 Antibody Cocktail Significantly Reduced Virus Levels and Need for Further Medical Attention” (2020).
11. Lilly, “Lilly's neutralizing antibody bamlanivimab (LY-CoV555) receives FDA emergency use authorization for the treatment of recently diagnosed COVID-19” (2020).
12. Regeneron, “Regeneron's Casirivimab and Imdevimab Antibody Cocktail for COVID-19 is First Combination Therapy to Receive FDA Emergency Use Authorization” (2020).
13. S. Ahangarzadeh, et al., An update on antiviral antibody-based biopharmaceuticals. Int. Immunopharmacol. 86 (2020).
14. A. Baum, et al., Antibody cocktail to SARS-CoV-2 spike protein prevents rapid mutational escape seen with individual antibodies. Science 0831, eabd0831 (2020).
15. A. M. Merchant, et al., An efficient route to human bispecific IgG. Nat. Biotechnol. 16, 677-681 (1998).
16. K. D. Orcutt, et al., A modular IgG-scFv bispecific antibody topology. Protein Eng. Des. Sel. 23, 221-228 (2010).
17. R. J. Boado, J. Z. Lu, E. K. W. Hui, W. M. Pardridge, IgG-single chain Fv fusion protein therapeutic for Alzheimer's disease: Expression in CHO cells and pharmacokinetics and brain delivery in the Rhesus monkey. Biotechnol. Bioeng. 105, 627-635 (2010).
18. A. Steinmetz, et al., CODV-Ig, a universal bispecific tetravalent and multifunctional immunoglobulin format for medical applications. MAbs 8, 867-878 (2016).
19. P. Holliger, T. Prospero, G. Winter, “Diabodies”: Small bivalent and bispecific antibody fragments. Proc. Natl. Acad. Sci. U.S.A. 90, 6444-6448 (1993).
20. M. C. Veri, et al., Therapeutic control of B cell activation via recruitment of Fcγ receptor IIb (CD32B) inhibitory function with a novel bispecific antibody scaffold. Arthritis Rheum. 62, 1933-1943 (2010).
21. Z. A. Ahmad, et al., ScFv antibody: Principles and clinical application. Clin. Dev. Immunol. 2012 (2012).
22. S. Bournazos, A. Gazumyan, M. S. Seaman, M. C. Nussenzweig, J. V. Ravetch, Bispecific Anti-HIV-1 Antibodies with Enhanced Breadth and Potency. Cell 165, 1609-1620 (2016).
23. T. Moshoette, S. A. Ali, M. A. Papathanasopoulos, M. A. Killick, Engineering and characterising a novel, highly potent bispecific antibody iMab-CAP256 that targets HIV-1. Retrovirology 16, 1-12 (2019).
24. M. Zanin, et al., An Anti-H5N1 Influenza Virus FcDART Antibody Is a Highly Efficacious Therapeutic Agent and Prophylactic against H5N1 Influenza Virus Infection. J. Virol. 89, 4549-4561 (2015).
25. J. C. Frei, et al., Bispecific antibody affords complete post-exposure protection of mice from both Ebola (Zaire) and Sudan viruses. Sci. Rep. 6, 2-11 (2016).
26. M. Yuan, et al., A highly conserved cryptic epitope in the receptor-binding domains of SARS-CoV-2 and SARS-CoV. Science 368, 630-633 (2020).
27. L. Yurkovetskiy, et al., Structural and Functional Analysis of the D614G SARS-CoV-2 Spike Protein Variant. Cell 183, 739-751.e8 (2020).
28. S. F. Sia, et al., Pathogenesis and transmission of SARS-CoV-2 in golden hamsters. Nature 583, 834-838 (2020).
29. M. Imai, et al., Syrian hamsters as a small animal model for SARS-CoV-2 infection and countermeasure development. Proc. Natl. Acad. Sci. 117, 16587-16595 (2020).
30. H. X. Zhou, Quantitative account of the enhanced affinity of two linked scFvs specific for different epitopes on the same antigen. J. Mol. Biol. 329, 1-8 (2003).
31. J. Zhang, et al., Pentamerization of single-domain antibodies from phage libraries: A novel strategy for the rapid generation of high-avidity antibody reagents. J. Mol. Biol. 335, 49-56 (2004).
32. G. Vauquelin, S. J. Charlton, Exploring avidity: Understanding the potential gains in functional affinity and target residence time of bivalent and heterobivalent ligands. Br. J. Pharmacol. 168, 1771-1785 (2013).
33. E. C. Piccione, et al., A bispecific antibody targeting CD47 and CD20 selectively binds and eliminates dual antigen expressing lymphoma cells. MAbs 7, 946-956 (2015).
34. E. K. Wagner, X. Wang, A. Bui, J. A. Maynard, Synergistic neutralization of pertussis toxin by a bispecific antibody in vitro and in vivo. Clin. Vaccine Immunol. 23, 851-862 (2016).
35. T.-C. Chou, N. Martin, CompuSyn software for drug combinations and for general dose-effect analysis, and user's guide. ComboSyn, Inc. Paramus, NJ 2007. [www.combosyn.com] (2007).
36. T. C. Chou, P. Talalay, Quantitative analysis of dose-effect relationships: the combined effects of multiple drugs or enzyme inhibitors. Adv. Enzyme Regul. 22, 27-55 (1984).
37. T. C. Chou, Theoretical basis, experimental design, and computerized simulation of synergism and antagonism in drug combination studies. Pharmacol. Rev. 58, 621-681 (2006).
38. A. Z. Wec, et al., Broad neutralization of SARS-related viruses by human monoclonal antibodies. Science 369, 731-736 (2020).
39. K. H. Dinnon, et al., A mouse-adapted model of SARS-CoV-2 to test COVID-19 countermeasures. Nature 586, 560-566 (2020).
40. Y. J. Hou, et al., SARS-CoV-2 Reverse Genetics Reveals a Variable Infection Gradient in the Respiratory Tract. Cell 182, 429-446.el4 (2020).
41. J. Sui, et al., Potent neutralization of severe acute respiratory syndrome (SARS) coronavirus by a human mAb to S1 protein that blocks receptor association. Proc. Natl. Acad. Sci. 101, 2536-2541 (2004).
42. X. C. Tang, et al., Identification of human neutralizing antibodies against MERS-CoV and their role in virus adaptive evolution. Proc. Natl. Acad. Sci. 111 (2014).
43. J. Huo, et al., Neutralization of SARS-CoV-2 by Destruction of the Prefusion Spike. Cell Host Microbe 28, 445-454.e6 (2020).
44. C. J. Bracken, et al., Bi-paratopic and multivalent V_{H} domains block ACE2 binding and neutralize SARS-CoV-2. Nat. Chem. Biol. (2020) https:/doi.org/10.1038/s41589-020-00679-1.
45. T. F. Custódio, et al., Selection, biophysical and structural analysis of synthetic nanobodies that effectively neutralize SARS-CoV-2. Nat. Commun. 11 (2020).
46. Y. Xiang, et al., Versatile and multivalent nanobodies efficiently neutralize SARS-CoV-2. Science 4747 (2020).
47. T. Grant, A. Rohou, N. Grigorieff, cisTEM, user-friendly software for single-particle image processing. Elife 7, e35383 (2018).
48. J. Dunbar, et al., SAbPred: a structure-based antibody prediction server. Nucleic Acids Res. 44, W474-W478 (2016).
49. C. O. Barnes, et al., SARS-CoV-2 neutralizing antibody structures inform therapeutic strategies. Nature (2020) https:/doi.org/10.1038/s41586-020-2852-1.
50. L. J. Beverly, W. W. Lockwood, P. P. Shah, H. Erdjument-Bromage, H. Varmus, Ubiquitination, localization, and stability of an anti-apoptotic BCL2-like protein, BCL2L10/BCLb, are regulated by Ubiquilin1. Proc. Natl. Acad. Sci. U.S.A. 109 (2012).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated multispecific antibody, wherein the antibody binds to at least one epitope in the receptor binding domain (RBD) of the spike protein (S) of a Severe Acute Respiratory Syndrome coronavirus (SARS-CoV2), and neutralizes SARS-CoV2.

2.-6. (canceled)

7. The antibody of claim 1, wherein the antibody is a fully human antibody.

8. (canceled)

9. The antibody of claim 1, wherein the antibody comprises:
a. a heavy chain with three CDRs comprising the amino acid sequences GGSIRTHS (SEQ ID NO:93), IHHSGAT (SEQ ID NO:94), and ARGPGILSY (SEQ ID NO:95) respectively, and/or a light chain with three CDRs comprising the amino acid sequences SSNIGSNT (SEQ ID NO:227), SNN (SEQ ID NO:228), and AAWDDSLNVHYV (SEQ ID NO:229) respectively;
b. a heavy chain with three CDRs comprising the amino acid sequences GGSISSYY (SEQ ID NO:96), IYTSGST (SEQ ID NO:97), and ARDVGFGWFDR (SEQ ID NO:98) respectively and/or a light chain with three CDRs comprising the amino acid sequences SGSIASNY (SEQ ID NO:230), EDN (SEQ ID NO:231), and QSFDSASLWV (SEQ ID NO:232) respectively;
c. a heavy chain with three CDRs comprising the amino acid sequences GGSIRTHS (SEQ ID NO:99), IHHSGAT (SEQ ID NO:100), and ARGPGILSY (SEQ ID NO:101) respectively and/or a light chain with three CDRs comprising the amino acid sequences SSNIGSND (SEQ ID NO:233), SNN (SEQ ID NO:234), and ATWDDSLSAGV (SEQ ID NO:235) respectively;
d. a heavy chain with three CDRs comprising the amino acid sequences GDSVSSYSDA (SEQ ID NO:102), TYYRSKWYN (SEQ ID NO:103), and AREIVATTPFRNYYYGMDV (SEQ ID NO:104) respectively and/or a light chain with three CDRs comprising the amino acid sequences SGSIASNY (SEQ ID NO:236), QDK (SEQ ID NO:237), and QSYDSSSLWV (SEQ ID NO:238) respectively;
e. a heavy chain with three CDRs comprising the amino acid sequences GFTFSHYD (SEQ ID NO:105), IGYDGTNL (SEQ ID NO:106), and ARAANYYDSSGYGRADAFDI (SEQ ID NO:107) respectively and/or a light chain with three CDRs comprising the amino acid sequences TGSIAGNY (SEQ ID NO:239), DDN (SEQ ID NO:240), and QSYDSGNRGV (SEQ ID NO:241) respectively;
f. a heavy chain with three CDRs comprising the amino acid sequences GFTFSDFP (SEQ ID NO:108), ISYDGNIK (SEQ ID NO:109), and A ARGGSSFDI (SEQ ID NO:110) respectively and/or a light chain with three CDRs comprising the amino acid sequences TSNIGNNA (SEQ ID NO:242), YNE (SEQ ID NO:243), and AAWDDSLSGHVV (SEQ ID NO:244) respectively;
g. a heavy chain with three CDRs comprising the amino acid sequences GFSLSTTGVG (SEQ ID NO:111), IYWNDDK (SEQ ID NO: 112), and ARISGSGYFYPFDI (SEQ ID NO:113) respectively and/or a light chain with three CDRs comprising the amino acid sequences SGSIASNY (SEQ ID NO:245), EDN (SEQ ID NO:246), and QSYDSSSLWV (SEQ ID NO:247) respectively;
h. a heavy chain with three CDRs comprising the amino acid sequences GYTFSDYY (SEQ ID NO:120), IDPNSGGT (SEQ ID NO:121), and ARDRGRGGQAGAFDY (SEQ ID NO:978) respectively and/or a light chain with three CDRs comprising the amino acid sequences KIGSKS (SEQ ID NO:254), DDS (SEQ ID NO:255), and HVWDSSSDQNV (SEQ ID NO:256) respectively;
i. a heavy chain with three CDRs comprising the amino acid sequences GFTFSSYA (SEQ ID NO:122), ISYGGSNK (SEQ ID NO:123), and AKVRGSGWYWGSAFDI (SEQ ID NO:124) respectively and/or a light chain with three CDRs comprising the amino acid sequences SLRAYF (SEQ ID NO:257), GQD (SEQ ID NO:258), and NSRDSGENHLI (SEQ ID NO:259) respectively;
j. a heavy chain with three CDRs comprising the amino acid sequences GYSFTGSH (SEQ ID NO:125), INPDSGVI (SEQ ID NO:126), and ARDKAIGYVWALDY (SEQ ID NO:127) respectively and/or a light chain with three CDRs comprising the amino acid sequences SSDVGTYNR (SEQ ID NO:260), EVS (SEQ ID NO:261), and SSYTRTFTYV (SEQ ID NO:262) respectively;
k. a heavy chain with three CDRs comprising the amino acid sequences GVSLDTIGMR (SEQ ID NO:128), IDWDDDK (SEQ ID NO:129), and ARSGLLYDLDV (SEQ ID NO:130) respectively and/or a light chain with three CDRs comprising the amino acid sequences DSDIGANF (SEQ ID NO:263), RNT (SEQ ID NO:264), and QSYDSSLSAYV (SEQ ID NO:265) respectively;
l. a heavy chain with three CDRs comprising the amino acid sequences GYSFTSYW (SEQ ID NO:134), IYPGDSDT (SEQ ID NO:135), and ARGWQWHDY (SEQ ID NO:136) respectively and/or a light chain with three CDRs comprising the amino acid sequences SLRSYY (SEQ ID NO:269), DKD (SEQ ID NO:270), and NSRDRSDNHVV (SEQ ID NO:271) respectively;
m. a heavy chain with three CDRs comprising the amino acid sequences GDSVSSRSSA (SEQ ID NO:137), TYYRSNWNY (SEQ ID NO:138), and VRNMRPDFDL (SEQ ID NO:139) respectively and/or a light chain with three CDRs comprising the amino acid sequences QSVSNN (SEQ ID NO:272), DAT (SEQ ID NO:273), and QQYDNLPV (SEQ ID NO:274) respectively;
n. a heavy chain with three CDRs comprising the amino acid sequences GYTFTTSG (SEQ ID NO:140), ISAYNGNT (SEQ ID NO:141), and ARDFHLYYGMIDV (SEQ ID NO:142) respectively and/or a light chain with three CDRs comprising the amino acid sequences SSDVGAYNY (SEQ ID NO:275), DVT (SEQ ID NO:276), and AVWDDGLNGRVV (SEQ ID NO:277) respectively;
o. a heavy chain with three CDRs comprising the amino acid sequences GGTFSSYA (SEQ ID NO:143), INPNSGGT (SEQ ID NO:144), and ARGSGGYYLG (SEQ ID NO:145) respectively and/or a light chain with three CDRs comprising the amino acid sequences SNNVGNQG (SEQ ID NO:278), MNN (SEQ ID NO:279), and SAWDSSLSRWV (SEQ ID NO:280) respectively;
p. a heavy chain with three CDRs comprising the amino acid sequences GGTFSSYT (SEQ ID NO:146), IIPILGTP (SEQ ID NO:147), and AVGSGWYSGFDY (SEQ ID NO:148) respectively and/or a light chain with three CDRs comprising the amino acid sequences SGSIASNY (SEQ ID NO:281), EDS (SEQ ID NO:282), and QSFHNSNPVI (SEQ ID NO:283) respectively;
q. a heavy chain with three CDRs comprising the amino acid sequences GFTFSSYW (SEQ ID NO:149), IKQDGSEK (SEQ ID NO:150), and ARGFYYYGAFDI (SEQ ID NO:151) respectively and/or a light chain with three CDRs comprising the amino acid sequences SGSIASNY (SEQ ID NO:284), EDN (SEQ ID NO:285), and QSYDSSNHWV (SEQ ID NO:286) respectively;
r. a heavy chain with three CDRs comprising the amino acid sequences GFTFDDYA (SEQ ID NO:152), IDWNSGVI (SEQ ID NO:153), and AKDAYSYGFLGAFDI (SEQ ID NO:154) respectively and/or a light chain with three CDRs comprising the amino acid sequences NIGSKS (SEQ ID NO:287), EDR (SEQ ID NO:288), and QVWDGDSDHYV (SEQ ID NO:289) respectively;
s. a heavy chain with three CDRs comprising the amino acid sequences GFTFDDYA (SEQ ID NO:155), IDWNSGVI (SEQ ID NO:156), and ARDILPSNFDGKKIIVFQPPAKRDLDNYYGMDV (SEQ ID NO:157) respectively and/or a light chain with three CDRs comprising the amino acid sequences SSDVGGYNL (SEQ ID NO:290), EGS (SEQ ID NO:291), and SSYTITDVVV (SEQ ID NO:292) respectively; or
t. a heavy chain with three CDRs comprising the amino acid sequences GYSFTSNW (SEQ ID NO:158), IFPGDSDT (SEQ ID NO:159), and ARESYNAYGS (SEQ ID NO:160) respectively and/or a light chain with three CDRs comprising the amino acid sequences SSNIGSNP (SEQ ID NO:293), SNN (SEQ ID NO:294), and AAWDDSLSGVV (SEQ ID NO:295) respectively.

10. The antibody of claim 1, wherein the antibody comprises:
a. a heavy chain with three CDRs comprising the amino acid sequences GFTFTTYG (SEQ ID NO:114), ISYDGSIK (SEQ ID NO:115), and ARVGDSSSYYGIDA (SEQ ID NO:116) respectively, and/or a light chain with three CDRs comprising the amino acid sequences SSNIGSNS (SEQ ID NO:248), SNN (SEQ ID NO:249), and AAWDDSLTGYV (SEQ ID NO:250) respectively;
b. a heavy chain with three CDRs comprising the amino acid sequences GFTFSSHA (SEQ ID NO:117), ISYDGSYT (SEQ ID NO:118), and ARDWVNFGMDV (SEQ ID NO:119) respectively, and/or a light chain with three CDRs comprising the amino acid sequences SSDVGGYNY (SEQ ID NO:251), EVS (SEQ ID NO:252), and AAWDDSLSGPV (SEQ ID NO:253) respectively; or
c. a heavy chain with three CDRs comprising the amino acid sequences GFTFSDYP (SEQ ID NO:131), TSYDGRIK (SEQ ID NO:132), and ARDPGWLRSVGMDV (SEQ ID NO:133) respectively, and/or a light chain with three CDRs comprising the amino acid sequences SGSIARNY (SEQ ID NO:266), ADR (SEQ ID NO:267), and QSYDSSNQAAV (SEQ ID NO:268) respectively.

11. The antibody of claim 1, wherein the antibody comprises:
a. a heavy chain with three CDRs comprising the amino acid sequences GYTFTSYG (SEQ ID NO:161), ISAYNGNT (SEQ ID NO:162), and ARGFPQLGSDY (SEQ ID NO:163) respectively, and/or a light chain with three CDRs comprising the amino acid sequences SGSIASNY (SEQ ID NO:296), EDN (SEQ ID NO:297), and QSYDSTNWV (SEQ ID NO:298) respectively;
b. a heavy chain with three CDRs comprising the amino acid sequences GGTFSSYA (SEQ ID NO:164), ISGYNGNT (SEQ ID NO:165), and ARQMKDSGNYWEYYYYGMDV (SEQ ID NO:166) respectively, and/or a light chain with three CDRs comprising the amino acid sequences NIGSES (SEQ ID NO:299), EDR (SEQ ID NO:300), and QVWNPSGSLQYV (SEQ ID NO:301) respectively;
c. a heavy chain with three CDRs comprising the amino acid sequences GYTFTSYG (SEQ ID NO:167), ISTYNGNT (SEQ ID NO:168), and ARDVFGHFDY (SEQ ID NO:169) respectively, and/or a light chain with three CDRs comprising the amino acid sequences SGNIATNY (SEQ ID NO:302), EDN (SEQ ID NO:303), and KSYDDGNHV (SEQ ID NO:304) respectively;
d. a heavy chain with three CDRs comprising the amino acid sequences GFSLTTTGVS (SEQ ID NO:170), IHWDDDK (SEQ ID NO:171), and ASFIMTVYAEYFED (SEQ ID NO:172) respectively, and/or a light chain with three CDRs comprising the amino acid sequences QSVSSN (SEQ ID NO:305), DVS (SEQ ID NO:306), and QQRGVWPLT (SEQ ID NO:307) respectively;
e. a heavy chain with three CDRs comprising the amino acid sequences GFSLSTSAMC (SEQ ID NO:173), IDWDNDR (SEQ ID NO:174), and AHSPYDSIWGSFRPSVYYFDY (SEQ ID NO:175) respectively, and/or a light chain with three CDRs comprising the amino acid sequences SGSIVSSY (SEQ ID NO:308), EHN (SEQ ID NO:309), and QSYDSQNGV (SEQ ID NO:310) respectively;
f. a heavy chain with three CDRs comprising the amino acid sequences GFTFSDYY (SEQ ID NO:176), ISSSSSDT (SEQ ID NO:177), and AMPTREPAY (SEQ ID NO:178) respectively, and/or a light chain with three CDRs comprising the amino acid sequences SSDLGTYNY (SEQ ID NO:311), DVF (SEQ ID NO:312), and SSYTSSSTYV (SEQ ID NO:313) respectively;
g. a heavy chain with three CDRs comprising the amino acid sequences GFAFSDFP (SEQ ID NO:179), ISYDGSLK (SEQ ID NO:180), and AREGVSNSRPFDH (SEQ ID NO:181) respectively, and/or a light chain with three CDRs comprising the amino acid sequences SIGTKS (SEQ ID NO:314), DDD (SEQ ID NO:315), and QVWESDDDDLV (SEQ ID NO:316) respectively;
h. a heavy chain with three CDRs comprising the amino acid sequences GFTFSSYA (SEQ ID NO:182), ISSNGGST (SEQ ID NO:183), and TRDLWSGSADSFDI (SEQ ID NO:184) respectively, and/or a light chain with three CDRs comprising the amino acid sequences SLRRYY (SEQ ID NO:317), GKN (SEQ ID NO:318), and NSRDISDNQWQWI (SEQ ID NO:319) respectively;
i. a heavy chain with three CDRs comprising the amino acid sequences GFPFNAYY (SEQ ID NO:185), INQDGSEK (SEQ ID NO:186), and ARLYWWGMDV (SEQ ID NO:187) respectively, and/or a light chain with three CDRs comprising the amino acid sequences SSDVGGYKY (SEQ ID NO:320), DVN (SEQ ID NO:321), and SSYTGRMNLYV (SEQ ID NO:322) respectively;
j. a heavy chain with three CDRs comprising the amino acid sequences GFTFDDYA (SEQ ID NO:188), IDWNSGVI (SEQ ID NO:189), and AKDAYSYGFLGAFDI (SEQ ID NO:190) respectively, and/or a light chain with three CDRs comprising the amino acid sequences NIRTKG (SEQ ID NO:323), YAS (SEQ ID NO:324), and QVWDSSSDLVV (SEQ ID NO:325) respectively;
k. a heavy chain with three CDRs comprising the amino acid sequences GFTFDDYA (SEQ ID NO:191), ISWNSGSI (SEQ ID NO:192), and ARDWWGSIDH (SEQ ID NO:193) respectively, and/or a light chain with three CDRs comprising the amino acid sequences SSDVGGYDY (SEQ ID NO:326), DVS (SEQ ID NO:327), and SSYTSSSPVV (SEQ ID NO:328) respectively;
l. a heavy chain with three CDRs comprising the amino acid sequences GGSISSSNW (SEQ ID NO:194), IYHSGST (SEQ ID NO:195), and ARRGGTYHRGAFDI (SEQ ID NO:196) respectively, and/or a light chain with three CDRs comprising the amino acid sequences SRDVGSYDL (SEQ ID NO:329), EGS (SEQ ID NO:330), and SSYTSSNSLV (SEQ ID NO:331) respectively;
m. a heavy chain with three CDRs comprising the amino acid sequences GASISNSF (SEQ ID NO:197), TSYSGNS (SEQ ID NO:198), and ARREWIKGHFDY (SEQ ID NO:199) respectively, and/or a light chain with three CDRs comprising the amino acid sequences GGSIASNY (SEQ ID NO:332), EDN (SEQ ID NO:333), and QSYDSSNPVV (SEQ ID NO:334) respectively;
n. a heavy chain with three CDRs comprising the amino acid sequences GGSFTTHS (SEQ ID NO:200), ILPGGAT (SEQ ID NO:201), and ARGPGILSY (SEQ ID NO:202) respectively, and/or a light chain with three CDRs comprising the amino acid sequences SSIGSND (SEQ ID NO:335), SNN (SEQ ID NO:336), and AWDDSLSAVV (SEQ ID NO:337) respectively;
o. a heavy chain with three CDRs comprising the amino acid sequences GGSFRTHS (SEQ ID NO:203), IHHSGAT (SEQ ID NO:204), and ARGPGILSY (SEQ ID NO:205) respectively, and/or a light chain with three CDRs comprising the amino acid sequences SSNIGSNT (SEQ ID NO:338), INN (SEQ ID NO:339), and AEWYDSLNVHYV (SEQ ID NO:340) respectively;
p. a heavy chain with three CDRs comprising the amino acid sequences GGSIRTHS (SEQ ID NO:206), IHHSGAT (SEQ ID NO:207), and ARGPGILSY (SEQ ID NO:208) respectively, and/or a light chain with three CDRs comprising the amino acid sequences SSNIGSNT (SEQ ID NO:341), INN (SEQ ID NO:342), and AECYDSLNDHYV (SEQ ID NO:343) respectively;
q. a heavy chain with three CDRs comprising the amino acid sequences GGSIRTHS (SEQ ID NO:209), IHHSGAT (SEQ ID NO:210), and GRGPGILSY (SEQ ID NO:211) respectively, and/or a light chain with three CDRs comprising the amino acid sequences SSNIGSNT (SEQ ID NO:344), SNN (SEQ ID NO:345), and AAWDDSLNVHYV (SEQ ID NO:346) respectively;
r. a heavy chain with three CDRs comprising the amino acid sequences GYSFTSYW (SEQ ID NO:212), IYPGDSDT (SEQ ID NO:213), and ARQGDGGGYDY (SEQ ID NO:214) respectively, and/or a light chain with three CDRs comprising the amino acid sequences SSNIGSNP (SEQ ID NO:347), NNN (SEQ ID NO:348), and AAWDDSLNGL (SEQ ID NO:349) respectively;
s. a heavy chain with three CDRs comprising the amino acid sequences RYSFSNYW (SEQ ID NO:215), IYPYDSDT (SEQ ID NO:216), and ARQGSSQSFDI (SEQ ID NO:217) respectively, and/or a light chain with three CDRs comprising the amino acid sequences SLRSYY (SEQ ID NO:350), GKN (SEQ ID NO:351), and NSRDSSGDVRV (SEQ ID NO:352) respectively;
t. a heavy chain with three CDRs comprising the amino acid sequences GYSFTSYW (SEQ ID NO:218), IYPGDSDT (SEQ ID NO:219), and ARRRGSAAAFDT (SEQ ID NO:220) respectively, and/or a light chain with three CDRs comprising the amino acid sequences SSNIGSNP (SEQ ID NO:353), DNN (SEQ ID NO:354), and EAWDDSLSGPV (SEQ ID NO:355) respectively;
u. a heavy chain with three CDRs comprising the amino acid sequences GYSFTSYW (SEQ ID NO:221), IYPGDSDT (SEQ ID NO:222), and ARTTYSYGSFDY (SEQ ID NO:223) respectively, and/or a light chain with three CDRs comprising the amino acid sequences SSNIGGNS (SEQ ID NO:356), RNN (SEQ ID NO:357), and AAWDDSLNGWV (SEQ ID NO:358) respectively;
v. a heavy chain with three CDRs comprising the amino acid sequences GDSVTSNSAA (SEQ ID NO:224), TYYSSKWYN (SEQ ID NO:225), and ARGWLRLSFDP (SEQ ID NO:226) respectively, and/or a light chain with three CDRs comprising the amino acid sequences SGSIASNY (SEQ ID NO:359), EDN (SEQ ID NO:360), and QSYDPNNHGVV (SEQ ID NO:361) respectively; or
w. a heavy chain with three CDRs comprising the amino acid sequences GFSLTTSGVS (SEQ ID NO:983), IHWDDDK (SEQ ID NO:984), and ASFIMTVYAEYFED (SEQ ID NO:985) respectively, and/or a light chain with three CDRs comprising the amino acid sequences QSVSSN (SEQ ID NO:986), DVS (SEQ ID NO:987), and QQRGAWPLT (SEQ ID NO:988) respectively.

12. The antibody of claim 1, wherein the antibody comprises:
a. a V_{H} amino acid sequence having SEQ ID NO: 1, and a V_{L} amino acid sequence having SEQ ID NO: 2;
b. a V_{H} amino acid sequence having SEQ ID NO: 3, and a V_{L} amino acid sequence having SEQ ID NO: 4;
c. a V_{H} amino acid sequence having SEQ ID NO: 5, and a V_{L} amino acid sequence having SEQ ID NO: 6;
d. a V_{H} amino acid sequence having SEQ ID NO: 7, and a V_{L} amino acid sequence having SEQ ID NO: 8;
e. a V_{H} amino acid sequence having SEQ ID NO: 9, and a V_{L} amino acid sequence having SEQ ID NO: 10;
f. a V_{H} amino acid sequence having SEQ ID NO: 11, and a V_{L} amino acid sequence having SEQ ID NO: 12;
g. a V_{H} amino acid sequence having SEQ ID NO: 13, and a V_{L} amino acid sequence having SEQ ID NO: 14;
h. a V_{H} amino acid sequence having SEQ ID NO: 19, and a V_{L} amino acid sequence having SEQ ID NO: 20;
i. a V_{H} amino acid sequence having SEQ ID NO: 21, and a V_{L} amino acid sequence having SEQ ID NO: 22;
j. a V_{H} amino acid sequence having SEQ ID NO: 23, and a V_{L} amino acid sequence having SEQ ID NO: 24;
k. a V_{H} amino acid sequence having SEQ ID NO: 25, and a V_{L} amino acid sequence having SEQ ID NO: 26;
l. a V_{H} amino acid sequence having SEQ ID NO: 29, and a V_{L} amino acid sequence having SEQ ID NO: 30;
m. a V_{H} amino acid sequence having SEQ ID NO: 31, and a V_{L} amino acid sequence having SEQ ID NO: 32;
n. a V_{H} amino acid sequence having SEQ ID NO: 33, and a V_{L} amino acid sequence having SEQ ID NO: 34;
o. a V_{H} amino acid sequence having SEQ ID NO: 35, and a V_{L} amino acid sequence having SEQ ID NO: 36;
p. a V_{H} amino acid sequence having SEQ ID NO: 37, and a V_{L} amino acid sequence having SEQ ID NO: 38;
q. a V_{H} amino acid sequence having SEQ ID NO: 39, and a V_{L} amino acid sequence having SEQ ID NO: 40;
r. a V_{H} amino acid sequence having SEQ ID NO: 41, and a V_{L} amino acid sequence having SEQ ID NO: 42;
s. a V_{H} amino acid sequence having SEQ ID NO: 43, and a V_{L} amino acid sequence having SEQ ID NO: 44; or
t. a V_{H} amino acid sequence having SEQ ID NO: 47, and a V_{L} amino acid sequence having SEQ ID NO: 48.

13. The antibody of claim 1, wherein the antibody comprises:
a. a V_{H} amino acid sequence having SEQ ID NO: 15, and a V_{L} amino acid sequence having SEQ ID NO: 16;
b. a V_{H} amino acid sequence having SEQ ID NO: 17, and a V_{L} amino acid sequence having SEQ ID NO: 18; or
c. a V_{H} amino acid sequence having SEQ ID NO: 27, and a V_{L} amino acid sequence having SEQ ID NO: 28.

14. The antibody of claim 1, wherein the antibody comprises:
a. a V_{H} amino acid sequence having SEQ ID NO: 49, and a V_{L} amino acid sequence having SEQ ID NO: 50;
b. a V_{H} amino acid sequence having SEQ ID NO: 51, and a V_{L} amino acid sequence having SEQ ID NO: 52;
c. a V_{H} amino acid sequence having SEQ ID NO: 53, and a V_{L} amino acid sequence having SEQ ID NO: 54;
d. a V_{H} amino acid sequence having SEQ ID NO: 55, and a V_{L} amino acid sequence having SEQ ID NO: 56;
e. a V_{H} amino acid sequence having SEQ ID NO: 57, and a V_{L} amino acid sequence having SEQ ID NO: 58;
f. a V_{H} amino acid sequence having SEQ ID NO: 59, and a V_{L} amino acid sequence having SEQ ID NO: 60;
g. a V_{H} amino acid sequence having SEQ ID NO: 61, and a V_{L} amino acid sequence having SEQ ID NO: 62;
h. a V_{H} amino acid sequence having SEQ ID NO: 63, and a V_{L} amino acid sequence having SEQ ID NO: 64;
i. a V_{H} amino acid sequence having SEQ ID NO: 65, and a V_{L} amino acid sequence having SEQ ID NO: 66;
j. a V_{H} amino acid sequence having SEQ ID NO: 67, and a V_{L} amino acid sequence having SEQ ID NO: 68;
k. a V_{H} amino acid sequence having SEQ ID NO: 69, and a V_{L} amino acid sequence having SEQ ID NO: 70;
l. a V_{H} amino acid sequence having SEQ ID NO: 71, and a V_{L} amino acid sequence having SEQ ID NO: 72;
m. a V_{H} amino acid sequence having SEQ ID NO: 73, and a V_{L} amino acid sequence having SEQ ID NO: 74;
n. a V_{H} amino acid sequence having SEQ ID NO: 75, and a V_{L} amino acid sequence having SEQ ID NO: 76;
o. a V_{H} amino acid sequence having SEQ ID NO: 77, and a V_{L} amino acid sequence having SEQ ID NO: 78;
p. a V_{H} amino acid sequence having SEQ ID NO: 79, and a V_{L} amino acid sequence having SEQ ID NO: 80;
q. a V_{H} amino acid sequence having SEQ ID NO: 81, and a V_{L} amino acid sequence having SEQ ID NO: 82;
r. a V_{H} amino acid sequence having SEQ ID NO: 83, and a V_{L} amino acid sequence having SEQ ID NO: 84;
s. a V_{H} amino acid sequence having SEQ ID NO: 85, and a V_{L} amino acid sequence having SEQ ID NO: 86;
t. a V_{H} amino acid sequence having SEQ ID NO: 87, and a V_{L} amino acid sequence having SEQ ID NO: 88;
u. a V_{H} amino acid sequence having SEQ ID NO: 89, and a V_{L} amino acid sequence having SEQ ID NO: 90;
v. a V_{H} amino acid sequence having SEQ ID NO: 91, and a V_{L} amino acid sequence having SEQ ID NO: 92; or
w. a V_{H} amino acid sequence having SEQ ID NO: 981, and a V_{L} amino acid sequence having SEQ ID NO: 982.

15. An isolated scFv multispecific antibody, wherein the antibody binds to at least one epitope in the receptor binding domain (RBD) of the spike protein of a Severe Acute Respiratory Syndrome coronavirus (SARS-CoV2), and neutralizes SARS-CoV2.

16.-20. (canceled)

21. The antibody of claim 15, wherein the antibody is a fully human antibody.

22. (canceled)

23. The antibody of claim 15, wherein the antibody comprises:
a. a heavy chain with three CDRs comprising the amino acid sequences GGSIRTHS (SEQ ID NO:93), IHHSGAT (SEQ ID NO:94), and ARGPGILSY (SEQ ID NO:95) respectively, and/or a light chain with three CDRs comprising the amino acid sequences SSNIGSNT (SEQ ID NO:227), SNN (SEQ ID NO:228), and AAWDDSLNVHYV (SEQ ID NO:229) respectively;
b. a heavy chain with three CDRs comprising the amino acid sequences GGSISSYY (SEQ ID NO:96), IYTSGST (SEQ ID NO:97), and ARDVGFGWFDR (SEQ ID NO:98) respectively and/or a light chain with three CDRs comprising the amino acid sequences SGSIASNY (SEQ ID NO:230), EDN (SEQ ID NO:231), and QSFDSASLWV (SEQ ID NO:232) respectively;
c. a heavy chain with three CDRs comprising the amino acid sequences GGSIRTHS (SEQ ID NO:99), IHHSGAT (SEQ ID NO:100), and ARGPGILSY (SEQ ID NO:101) respectively and/or a light chain with three CDRs comprising the amino acid sequences SSNIGSND (SEQ ID NO:233), SNN (SEQ ID NO:234), and ATWDDSLSAGV (SEQ ID NO:235) respectively;
d. a heavy chain with three CDRs comprising the amino acid sequences GDSVSSYSDA (SEQ ID NO:102), TYYRSKWYN (SEQ ID NO:103), and AREIVATTPFRNYYYGMDV (SEQ ID NO:104) respectively and/or a light chain with three CDRs comprising the amino acid sequences SGSIASNY (SEQ ID NO:236), QDK (SEQ ID NO:237), and QSYDSSSLWV (SEQ ID NO:238) respectively;
e. a heavy chain with three CDRs comprising the amino acid sequences GFTFSHYD (SEQ ID NO:105), IGYDGTNL (SEQ ID NO:106), and ARAANYYDSSGYGRADAFDI (SEQ ID NO:107) respectively and/or a light chain with three CDRs comprising the amino acid sequences TGSIAGNY (SEQ ID NO:239), DDN (SEQ ID NO:240), and QSYDSGNRGV (SEQ ID NO:241) respectively;
f. a heavy chain with three CDRs comprising the amino acid sequences GFTFSDFP (SEQ ID NO:108), ISYDGNIK (SEQ ID NO:109), and A ARGGSSFDI (SEQ ID NO:110) respectively and/or a light chain with three CDRs comprising the amino acid sequences TSNIGNNA (SEQ ID NO:242), YNE (SEQ ID NO:243), and AAWDDSLSGHVV (SEQ ID NO:244) respectively;
g. a heavy chain with three CDRs comprising the amino acid sequences GFSLSTTGVG (SEQ ID NO:111), IYWNDDK (SEQ ID NO: 112), and ARISGSGYFYPFDI (SEQ ID NO:113) respectively and/or a light chain with three CDRs comprising the amino acid sequences SGSIASNY (SEQ ID NO:245), EDN (SEQ ID NO:246), and QSYDSSSLWV (SEQ ID NO:247) respectively;
h. a heavy chain with three CDRs comprising the amino acid sequences GYTFSDYY (SEQ ID NO:120), IDPNSGGT (SEQ ID NO:121), and ARDRGRGGQAGAFDY (SEQ ID NO:978) respectively and/or a light chain with three CDRs comprising the amino acid sequences KIGSKS (SEQ ID NO:254), DDS (SEQ ID NO:255), and HVWDSSSDQNV (SEQ ID NO:256) respectively;
i. a heavy chain with three CDRs comprising the amino acid sequences GFTFSSYA (SEQ ID NO:122), ISYGGSNK (SEQ ID NO:123), and AKVRGSGWYWGSAFDI (SEQ ID NO:124) respectively and/or a light chain with three CDRs comprising the amino acid sequences SLRAYF (SEQ ID NO:257), GQD (SEQ ID NO:258), and NSRDSGENHLI (SEQ ID NO:259) respectively;
j. a heavy chain with three CDRs comprising the amino acid sequences GYSFTGSH (SEQ ID NO:125), INPDSGVI (SEQ ID NO:126), and ARDKAIGYVWALDY (SEQ ID NO:127) respectively and/or a light chain with three CDRs comprising the amino acid sequences SSDVGTYNR (SEQ ID NO:260), EVS (SEQ ID NO:261), and SSYTRTFTYV (SEQ ID NO:262) respectively;
k. a heavy chain with three CDRs comprising the amino acid sequences GVSLDTIGMR (SEQ ID NO:128), IDWDDDK (SEQ ID NO:129), and ARSGLLYDLDV (SEQ ID NO:130) respectively and/or a light chain with three CDRs comprising the amino acid sequences DSDIGANF (SEQ ID NO:263), RNT (SEQ ID NO:264), and QSYDSSLSAYV (SEQ ID NO:265) respectively;
l. a heavy chain with three CDRs comprising the amino acid sequences GYSFTSYW (SEQ ID NO:134), IYPGDSDT (SEQ ID NO:135), and ARGWQWHDY (SEQ ID NO:136) respectively and/or a light chain with three CDRs comprising the amino acid sequences SLRSYY (SEQ ID NO:269), DKD (SEQ ID NO:270), and NSRDRSDNHVV (SEQ ID NO:271) respectively;
m. a heavy chain with three CDRs comprising the amino acid sequences GDSVSSRSSA (SEQ ID NO:137), TYYRSNWNY (SEQ ID NO:138), and VRNMRPDFDL (SEQ ID NO:139) respectively and/or a light chain with three CDRs comprising the amino acid sequences QSVSNN (SEQ ID NO:272), DAT (SEQ ID NO:273), and QQYDNLPV (SEQ ID NO:274) respectively;
n. a heavy chain with three CDRs comprising the amino acid sequences GYTFTTSG (SEQ ID NO:140), ISAYNGNT (SEQ ID NO:141), and ARDFHLYYGMDV (SEQ ID NO:142) respectively and/or a light chain with three CDRs comprising the amino acid sequences SSDVGAYNY (SEQ ID NO:275), DVT (SEQ ID NO:276), and AVWDDGLNGRVV (SEQ ID NO:277) respectively;
o. a heavy chain with three CDRs comprising the amino acid sequences GGTFSSYA (SEQ ID NO:143), INPNSGGT (SEQ ID NO:144), and ARGSGGYYLG (SEQ ID NO:145) respectively and/or a light chain with three CDRs comprising the amino acid sequences SNNVGNQG (SEQ ID NO:278), MNN (SEQ ID NO:279), and SAWDSSLSRWV (SEQ ID NO:280) respectively;
p. a heavy chain with three CDRs comprising the amino acid sequences GGTFSSYT (SEQ ID NO:146), IIPILGTP (SEQ ID NO:147), and AVGSGWYSGFDY (SEQ ID NO:148) respectively and/or a light chain with three CDRs comprising the amino acid sequences SGSIASNY (SEQ ID NO:281), EDS (SEQ ID NO:282), and QSFHNSNPVI (SEQ ID NO:283) respectively;
q. a heavy chain with three CDRs comprising the amino acid sequences GFTFSSYW (SEQ ID NO:149), IKQDGSEK (SEQ ID NO:150), and ARGFYYYGAFDI (SEQ ID NO:151) respectively and/or a light chain with three CDRs comprising the amino acid sequences SGSIASNY (SEQ ID NO:284), EDN (SEQ ID NO:285), and QSYDSSNHWV (SEQ ID NO:286) respectively;
r. a heavy chain with three CDRs comprising the amino acid sequences GFTFDDYA (SEQ ID NO:152), IDWNSGVI (SEQ ID NO:153), and AKDAYSYGFLGAFDI (SEQ ID NO:154) respectively and/or a light chain with three CDRs comprising the amino acid sequences NIGSKS (SEQ ID NO:287), EDR (SEQ ID NO:288), and QVWDGDSDHYV (SEQ ID NO:289) respectively;
s. a heavy chain with three CDRs comprising the amino acid sequences GFTFDDYA (SEQ ID NO:155), IDWNSGVI (SEQ ID NO:156), and ARDILPSNFDGKKIIVFQPPAKRDLDNYYGMDV (SEQ ID NO:157) respectively and/or a light chain with three CDRs comprising the amino acid sequences SSDVGGYNL (SEQ ID NO:290), EGS (SEQ ID NO:291), and SSYTITDVVV (SEQ ID NO:292) respectively; or
t. a heavy chain with three CDRs comprising the amino acid sequences GYSFTSNW (SEQ ID NO:158), IFPGDSDT (SEQ ID NO:159), and ARESYNAYGS (SEQ ID NO:160) respectively and/or a light chain with three CDRs comprising the amino acid sequences SSNIGSNP (SEQ ID NO:293), SNN (SEQ ID NO:294), and AAWDDSLSGVV (SEQ ID NO:295) respectively.

24. The antibody of claim 15, wherein the antibody comprises:
a. a heavy chain with three CDRs comprising the amino acid sequences GFTFTTYG (SEQ ID NO:114), ISYDGSIK (SEQ ID NO:115), and ARVGDSSSYYGIDA (SEQ ID NO:116) respectively, and/or a light chain with three CDRs comprising the amino acid sequences SSNIGSNS (SEQ ID NO:248), SNN (SEQ ID NO:249), and AAWDDSLTGYV (SEQ ID NO:250) respectively;
b. a heavy chain with three CDRs comprising the amino acid sequences GFTFSSHA (SEQ ID NO:117), ISYDGSYT (SEQ ID NO:118), and ARDWVNFGMDV (SEQ ID NO:119) respectively, and/or a light chain with three CDRs comprising the amino acid sequences SSDVGGYNY (SEQ ID NO:251), EVS (SEQ ID NO:252), and AAWDDSLSGPV (SEQ ID NO:253) respectively; or
c. a heavy chain with three CDRs comprising the amino acid sequences GFTFSDYP (SEQ ID NO:131), TSYDGRIK (SEQ ID NO:132), and ARDPGWLRSVGMDV (SEQ ID NO:133) respectively, and/or a light chain with three CDRs comprising the amino acid sequences SGSIARNY (SEQ ID NO:266), ADR (SEQ ID NO:267), and QSYDSSNQAAV (SEQ ID NO:268) respectively.

25. The antibody of claim 15, wherein the antibody comprises:
a. a heavy chain with three CDRs comprising the amino acid sequences GYTFTSYG (SEQ ID NO:161), ISAYNGNT (SEQ ID NO:162), and ARGFPQLGSDY (SEQ ID NO:163) respectively, and/or a light chain with three CDRs comprising the amino acid sequences SGSIASNY (SEQ ID NO:296), EDN (SEQ ID NO:297), and QSYDSTNWV (SEQ ID NO:298) respectively;
b. a heavy chain with three CDRs comprising the amino acid sequences GGTFSSYA (SEQ ID NO:164), ISGYNGNT (SEQ ID NO:165), and ARQMKDSGNYWEYYYYGMDV (SEQ ID NO:166) respectively, and/or a light chain with three CDRs comprising the amino acid sequences NIGSES (SEQ ID NO:299), EDR (SEQ ID NO:300), and QVWNPSGSLQYV (SEQ ID NO:301) respectively;
c. a heavy chain with three CDRs comprising the amino acid sequences GYTFTSYG (SEQ ID NO:167), ISTYNGNT (SEQ ID NO:168), and ARDVFGHFDY (SEQ ID NO:169) respectively, and/or a light chain with three CDRs comprising the amino acid sequences SGNIATNY (SEQ ID NO:302), EDN (SEQ ID NO:303), and KSYDDGNHV (SEQ ID NO:304) respectively;
d. a heavy chain with three CDRs comprising the amino acid sequences GFSLTTTGVS (SEQ ID NO:170), IHWDDDK (SEQ ID NO:171), and ASFIMTVYAEYFED (SEQ ID NO:172) respectively, and/or a light chain with three CDRs comprising the amino acid sequences QSVSSN (SEQ ID NO:305), DVS (SEQ ID NO:306), and QQRGVWPLT (SEQ ID NO:307) respectively;
e. a heavy chain with three CDRs comprising the amino acid sequences GFSLSTSAMC (SEQ ID NO:173), IDWDNDR (SEQ ID NO:174), and AHSPYDSIWGSFRPSVYYFDY (SEQ ID NO:175) respectively, and/or a light chain with three CDRs comprising the amino acid sequences SGSIVSSY (SEQ ID NO:308), EHN (SEQ ID NO:309), and QSYDSQNGV (SEQ ID NO:310) respectively;
f. a heavy chain with three CDRs comprising the amino acid sequences GFTFSDYY (SEQ ID NO:176), ISSSSSDT (SEQ ID NO:177), and AMPTREPAY (SEQ ID NO:178) respectively, and/or a light chain with three CDRs comprising the amino acid sequences SSDLGTYNY (SEQ ID NO:311), DVF (SEQ ID NO:312), and SSYTSSSTYV (SEQ ID NO:313) respectively;
g. a heavy chain with three CDRs comprising the amino acid sequences GFAFSDFP (SEQ ID NO:179), ISYDGSLK (SEQ ID NO:180), and AREGVSNSRPFDH (SEQ ID NO:181) respectively, and/or a light chain with three CDRs comprising the amino acid sequences SIGTKS (SEQ ID NO:314), DDD (SEQ ID NO:315), and QVWESDDDDLV (SEQ ID NO:316) respectively;
h. a heavy chain with three CDRs comprising the amino acid sequences GFTFSSYA (SEQ ID NO:182), ISSNGGST (SEQ ID NO:183), and TRDLWSGSADSFDI (SEQ ID NO:184) respectively, and/or a light chain with three CDRs comprising the amino acid sequences SLRRYY (SEQ ID NO:317), GKN (SEQ ID NO:318), and NSRDISDNQWQWI (SEQ ID NO:319) respectively;
i. a heavy chain with three CDRs comprising the amino acid sequences GFPFNAYY (SEQ ID NO:185), INQDGSEK (SEQ ID NO:186), and ARLYWWGMDV (SEQ ID NO:187) respectively, and/or a light chain with three CDRs comprising the amino acid sequences SSDVGGYKY (SEQ ID NO:320), DVN (SEQ ID NO:321), and SSYTGRMNLYV (SEQ ID NO:322) respectively;
j. a heavy chain with three CDRs comprising the amino acid sequences GFTFDDYA (SEQ ID NO:188), IDWNSGVI (SEQ ID NO:189), and AKDAYSYGFLGAFDI (SEQ ID NO:190) respectively, and/or a light chain with three CDRs comprising the amino acid sequences NIRTKG (SEQ ID NO:323), YAS (SEQ ID NO:324), and QVWDSSSDLVV (SEQ ID NO:325) respectively;
k. a heavy chain with three CDRs comprising the amino acid sequences GFTFDDYA (SEQ ID NO:191), ISWNSGSI (SEQ ID NO:192), and ARDWWGSIDH (SEQ ID NO:193) respectively, and/or a light chain with three CDRs comprising the amino acid sequences SSDVGGYDY (SEQ ID NO:326), DVS (SEQ ID NO:327), and SSYTSSSPVV (SEQ ID NO:328) respectively;
l. a heavy chain with three CDRs comprising the amino acid sequences GGSISSSNW (SEQ ID NO:194), IYHSGST (SEQ ID NO:195), and ARRGGTYHRGAFDI (SEQ ID NO:196) respectively, and/or a light chain with three CDRs comprising the amino acid sequences SRDVGSYDL (SEQ ID NO:329), EGS (SEQ ID NO:330), and SSYTSSNSLV (SEQ ID NO:331) respectively;
m. a heavy chain with three CDRs comprising the amino acid sequences GASISNSF (SEQ ID NO:197), TSYSGNS (SEQ ID NO:198), and ARREWIKGHFDY (SEQ ID NO:199) respectively, and/or a light chain with three CDRs comprising the amino acid sequences GGSIASNY (SEQ ID NO:332), EDN (SEQ ID NO:333), and QSYDSSNPVV (SEQ ID NO:334) respectively;
n. a heavy chain with three CDRs comprising the amino acid sequences GGSFTTHS (SEQ ID NO:200), ILPGGAT (SEQ ID NO:201), and ARGPGILSY (SEQ ID NO:202) respectively, and/or a light chain with three CDRs comprising the amino acid sequences SSIGSND (SEQ ID NO:335), SNN (SEQ ID NO:336), and AWDDSLSAVV (SEQ ID NO:337) respectively;
o. a heavy chain with three CDRs comprising the amino acid sequences GGSFRTHS (SEQ ID NO:203), IHHSGAT (SEQ ID NO:204), and ARGPGILSY (SEQ ID NO:205) respectively, and/or a light chain with three CDRs comprising the amino acid sequences SSNIGSNT (SEQ ID NO:338), INN (SEQ ID NO:339), and AEWYDSLNVHYV (SEQ ID NO:340) respectively;
p. a heavy chain with three CDRs comprising the amino acid sequences GGSIRTHS (SEQ ID NO:206), IHHSGAT (SEQ ID NO:207), and ARGPGILSY (SEQ ID NO:208) respectively, and/or a light chain with three CDRs comprising the amino acid sequences SSNIGSNT (SEQ ID NO:341), INN (SEQ ID NO:342), and AECYDSLNDHYV (SEQ ID NO:343) respectively;
q. a heavy chain with three CDRs comprising the amino acid sequences GGSIRTHS (SEQ ID NO:209), IHHSGAT (SEQ ID NO:210), and GRGPGILSY (SEQ ID NO:211) respectively, and/or a light chain with three CDRs comprising the amino acid sequences SSNIGSNT (SEQ ID NO:344), SNN (SEQ ID NO:345), and AAWDDSLNVHYV (SEQ ID NO:346) respectively;
r. a heavy chain with three CDRs comprising the amino acid sequences GYSFTSYW (SEQ ID NO:212), IYPGDSDT (SEQ ID NO:213), and ARQGDGGGYDY (SEQ ID NO:214) respectively, and/or a light chain with three CDRs comprising the amino acid sequences SSNIGSNP (SEQ ID NO:347), NNN (SEQ ID NO:348), and AAWDDSLNGL (SEQ ID NO:349) respectively;
s. a heavy chain with three CDRs comprising the amino acid sequences RYSFSNYW (SEQ ID NO:215), IYPYDSDT (SEQ ID NO:216), and ARQGSSQSFDI (SEQ ID NO:217) respectively, and/or a light chain with three CDRs comprising the amino acid sequences SLRSYY (SEQ ID NO:350), GKN (SEQ ID NO:351), and NSRDSSGDVRV (SEQ ID NO:352) respectively;
t. a heavy chain with three CDRs comprising the amino acid sequences GYSFTSYW (SEQ ID NO:218), IYPGDSDT (SEQ ID NO:219), and ARRRGSAAAFDT (SEQ ID NO:220) respectively, and/or a light chain with three CDRs comprising the amino acid sequences SSNIGSNP (SEQ ID NO:353), DNN (SEQ ID NO:354), and EAWDDSLSGPV (SEQ ID NO:355) respectively;
u. a heavy chain with three CDRs comprising the amino acid sequences GYSFTSYW (SEQ ID NO:221), IYPGDSDT (SEQ ID NO:222), and ARTTYSYGSFDY (SEQ ID NO:223) respectively, and/or a light chain with three CDRs comprising the amino acid sequences SSNIGGNS (SEQ ID NO:356), RNN (SEQ ID NO:357), and AAWDDSLNGWV (SEQ ID NO:358) respectively;
v. a heavy chain with three CDRs comprising the amino acid sequences GDSVTSNSAA (SEQ ID NO:224), TYYSSKWYN (SEQ ID NO:225), and ARGWLRLSFDP (SEQ ID NO:226) respectively, and/or a light chain with three CDRs comprising the amino acid sequences SGSIASNY (SEQ ID NO:359), EDN (SEQ ID NO:360), and QSYDPNNHGVV (SEQ ID NO:361) respectively; or
w. a heavy chain with three CDRs comprising the amino acid sequences GFSLTTSGVS (SEQ ID NO:983), IHWDDDK (SEQ ID NO:984), and ASFIMTVYAEYFED (SEQ ID NO:985) respectively, and/or a light chain with three CDRs comprising the amino acid sequences QSVSSN (SEQ ID NO:986), DVS (SEQ ID NO:987), and QQRGAWPLT (SEQ ID NO:988) respectively.

26. The antibody of claim 15, wherein the antibody comprises:
a. a V_{H} amino acid sequence having SEQ ID NO: 1, and a V_{L} amino acid sequence having SEQ ID NO: 2;
b. a V_{H} amino acid sequence having SEQ ID NO: 3, and a V_{L} amino acid sequence having SEQ ID NO: 4;
c. a V_{H} amino acid sequence having SEQ ID NO: 5, and a V_{L} amino acid sequence having SEQ ID NO: 6;
d. a V_{H} amino acid sequence having SEQ ID NO: 7, and a V_{L} amino acid sequence having SEQ ID NO: 8;
e. a V_{H} amino acid sequence having SEQ ID NO: 9, and a V_{L} amino acid sequence having SEQ ID NO: 10;
f. a V_{H} amino acid sequence having SEQ ID NO: 11, and a V_{L} amino acid sequence having SEQ ID NO: 12;
g. a V_{H} amino acid sequence having SEQ ID NO: 13, and a V_{L} amino acid sequence having SEQ ID NO: 14;
h. a V_{H} amino acid sequence having SEQ ID NO: 19, and a V_{L} amino acid sequence having SEQ ID NO: 20;
i. a V_{H} amino acid sequence having SEQ ID NO: 21, and a V_{L} amino acid sequence having SEQ ID NO: 22;
j. a V_{H} amino acid sequence having SEQ ID NO: 23, and a V_{L} amino acid sequence having SEQ ID NO: 24;
k. a V_{H} amino acid sequence having SEQ ID NO: 25, and a V_{L} amino acid sequence having SEQ ID NO: 26;
l. a V_{H} amino acid sequence having SEQ ID NO: 29, and a V_{L} amino acid sequence having SEQ ID NO: 30;
m. a V_{H} amino acid sequence having SEQ ID NO: 31, and a V_{L} amino acid sequence having SEQ ID NO: 32;
n. a V_{H} amino acid sequence having SEQ ID NO: 33, and a V_{L} amino acid sequence having SEQ ID NO: 34;
o. a V_{H} amino acid sequence having SEQ ID NO: 35, and a V_{L} amino acid sequence having SEQ ID NO: 36;
p. a V_{H} amino acid sequence having SEQ ID NO: 37, and a V_{L} amino acid sequence having SEQ ID NO: 38;
q. a V_{H} amino acid sequence having SEQ ID NO: 39, and a V_{L} amino acid sequence having SEQ ID NO: 40;
r. a V_{H} amino acid sequence having SEQ ID NO: 41, and a V_{L} amino acid sequence having SEQ ID NO: 42;
s. a V_{H} amino acid sequence having SEQ ID NO: 43, and a V_{L} amino acid sequence having SEQ ID NO: 44; or
t. a V_{H} amino acid sequence having SEQ ID NO: 47, and a V_{L} amino acid sequence having SEQ ID NO: 48.

27. The antibody of claim 15, wherein the antibody comprises:
a. a V_{H} amino acid sequence having SEQ ID NO: 15, and a V_{L} amino acid sequence having SEQ ID NO: 16;
b. a V_{H} amino acid sequence having SEQ ID NO: 17, and a V_{L} amino acid sequence having SEQ ID NO: 18; or
c. a V_{H} amino acid sequence having SEQ ID NO: 27, and a V_{L} amino acid sequence having SEQ ID NO: 28.

28. The antibody of claim 15, wherein the antibody comprises:
a. a V_{H} amino acid sequence having SEQ ID NO: 49, and a V_{L} amino acid sequence having SEQ ID NO: 50;
b. a V_{H} amino acid sequence having SEQ ID NO: 51, and a V_{L} amino acid sequence having SEQ ID NO: 52;
c. a V_{H} amino acid sequence having SEQ ID NO: 53, and a V_{L} amino acid sequence having SEQ ID NO: 54;
d. a V_{H} amino acid sequence having SEQ ID NO: 55, and a V_{L} amino acid sequence having SEQ ID NO: 56;
e. a V_{H} amino acid sequence having SEQ ID NO: 57, and a V_{L} amino acid sequence having SEQ ID NO: 58;
f. a V_{H} amino acid sequence having SEQ ID NO: 59, and a V_{L} amino acid sequence having SEQ ID NO: 60;
g. a V_{H} amino acid sequence having SEQ ID NO: 61, and a V_{L} amino acid sequence having SEQ ID NO: 62;
h. a V_{H} amino acid sequence having SEQ ID NO: 63, and a V_{L} amino acid sequence having SEQ ID NO: 64;
i. a V_{H} amino acid sequence having SEQ ID NO: 65, and a V_{L} amino acid sequence having SEQ ID NO: 66;
j. a V_{H} amino acid sequence having SEQ ID NO: 67, and a V_{L} amino acid sequence having SEQ ID NO: 68;
k. a V_{H} amino acid sequence having SEQ ID NO: 69, and a V_{L} amino acid sequence having SEQ ID NO: 70;
l. a V_{H} amino acid sequence having SEQ ID NO: 71, and a V_{L} amino acid sequence having SEQ ID NO: 72;
m. a V_{H} amino acid sequence having SEQ ID NO: 73, and a V_{L} amino acid sequence having SEQ ID NO: 74;
n. a V_{H} amino acid sequence having SEQ ID NO: 75, and a V_{L} amino acid sequence having SEQ ID NO: 76;
o. a V_{H} amino acid sequence having SEQ ID NO: 77, and a V_{L} amino acid sequence having SEQ ID NO: 78;
p. a V_{H} amino acid sequence having SEQ ID NO: 79, and a V_{L} amino acid sequence having SEQ ID NO: 80;
q. a V_{H} amino acid sequence having SEQ ID NO: 81, and a V_{L} amino acid sequence having SEQ ID NO: 82;
r. a V_{H} amino acid sequence having SEQ ID NO: 83, and a V_{L} amino acid sequence having SEQ ID NO: 84;
s. a V_{H} amino acid sequence having SEQ ID NO: 85, and a V_{L} amino acid sequence having SEQ ID NO: 86;
t. a V_{H} amino acid sequence having SEQ ID NO: 87, and a V_{L} amino acid sequence having SEQ ID NO: 88;
u. a V_{H} amino acid sequence having SEQ ID NO: 89, and a V_{L} amino acid sequence having SEQ ID NO: 90; or
v. a V_{H} amino acid sequence having SEQ ID NO: 91, and a V_{L} amino acid sequence having SEQ ID NO: 92.

29. The antibody of claim 1, wherein the antibody comprises:
a. a heavy chain with three CDRs comprising the amino acid sequences GFSLSTSGVG (SEQ ID NO:754), IYWDDDK (SEQ ID NO:755), and ARISGSGYFYPFDI (SEQ ID NO:756) respectively, and/or a light chain with three CDRs comprising the amino acid sequences SGSIASNY (SEQ ID NO:802), EDN (SEQ ID NO:803), and QSYDSSNLWV (SEQ ID NO:804) respectively;
b. a heavy chain with three CDRs comprising the amino acid sequences GDSVSSNSAA (SEQ ID NO:757), TYYRSRWYN (SEQ ID NO:758), and AREIRGFDY (SEQ ID NO:759) respectively, and/or a light chain with three CDRs comprising the amino acid sequences SSDVGAYNF (SEQ ID NO:805), DFN (SEQ ID NO:806), and SSYAGSNNFDVV (SEQ ID NO:807) respectively;
c. a heavy chain with three CDRs comprising the amino acid sequences GFTFGDYA (SEQ ID NO:760), IRSKAYGGTT (SEQ ID NO:761), and TTADDDMDV (SEQ ID NO:762) respectively, and/or a light chain with three CDRs comprising the amino acid sequences SGTIASNY (SEQ ID NO:808), EDN (SEQ ID NO:809), and QSYDTSNHYV (SEQ ID NO:810) respectively;
d. a heavy chain with three CDRs comprising the amino acid sequences GFTFSNYG (SEQ ID NO:763), IWERGSKK (SEQ ID NO:764), and AREGISMTGAEYFQH (SEQ ID NO:765) respectively, and/or a light chain with three CDRs comprising the amino acid sequences SSNIGAGYD (SEQ ID NO:811), GTN (SEQ ID NO:812), and QSYDNSLTDPYV (SEQ ID NO:813) respectively;
e. a heavy chain with three CDRs comprising the amino acid sequences GFTFDDYA (SEQ ID NO:766), IDWNSGVI (SEQ ID NO:767), and AKDIGPGGSGSYYAFDI (SEQ ID NO:768) respectively, and/or a light chain with three CDRs comprising the amino acid sequences SSDVGGSKY (SEQ ID NO:814), DVT (SEQ ID NO:815), and AAWDDSLNGVV (SEQ ID NO:816) respectively;
f. a heavy chain with three CDRs comprising the amino acid sequences GFSFSRYG (SEQ ID NO:769), IRHDGSKK (SEQ ID NO:770), and AKDGRLEAALDD (SEQ ID NO:771) respectively, and/or a light chain with three CDRs comprising the amino acid sequences SGSIANNF (SEQ ID NO:817), EDN (SEQ ID NO:818), and QSYDSSNLV (SEQ ID NO:819) respectively;
g. a heavy chain with three CDRs comprising the amino acid sequences GYSFTSYW (SEQ ID NO:772), IYPGDSDT (SEQ ID NO:773), and ARRGDLDAFDI (SEQ ID NO:774) respectively, and/or a light chain with three CDRs comprising the amino acid sequences SANIGSNA (SEQ ID NO:820), GNT (SEQ ID NO:821), and AAWDDSLNGYV (SEQ ID NO:822) respectively;
h. a heavy chain with three CDRs comprising the amino acid sequences GYRLSDYY (SEQ ID NO:775), IKQDGSEK (SEQ ID NO:776), and ARVRGWSRGYFDY (SEQ ID NO:777) respectively, and/or a light chain with three CDRs comprising the amino acid sequences SGSIASNY (SEQ ID NO:823), EDN (SEQ ID NO:824), and QSYDSSNHWV (SEQ ID NO:825) respectively;
i. a heavy chain with three CDRs comprising the amino acid sequences GFTFDDYA (SEQ ID NO:778), ISWNSGSI (SEQ ID NO:779), and ARDWWGSIDH (SEQ ID NO:780) respectively, and/or a light chain with three CDRs comprising the amino acid sequences SSDVGGYDY (SEQ ID NO:826), DVS (SEQ ID NO:827), and SSYTSSSPVV (SEQ ID NO:828) respectively;
j. a heavy chain with three CDRs comprising the amino acid sequences GFTFSHYD (SEQ ID NO:781), IGYDGTNL (SEQ ID NO:782), and ARAANYYDSSGYGRADAFDI (SEQ ID NO:783) respectively, and/or a light chain with three CDRs comprising the amino acid sequences TGSIAGNY (SEQ ID NO:829), DDN (SEQ ID NO:830), and QSYDSGNRGV (SEQ ID NO:831) respectively;
k. a heavy chain with three CDRs comprising the amino acid sequences GGTFSTYG (SEQ ID NO:784), IIPSLGIP (SEQ ID NO:785), and ARENIDLATNDF (SEQ ID NO:786) respectively, and/or a light chain with three CDRs comprising the amino acid sequences SRDIGAYGY (SEQ ID NO:832), EVR (SEQ ID NO:833), and SSYTSSSTLDVV (SEQ ID NO:834) respectively;
l. a heavy chain with three CDRs comprising the amino acid sequences GGTFSSSG (SEQ ID NO:787), IIPMLGTP (SEQ ID NO:788), and ARDGGNYDY (SEQ ID NO:789) respectively, and/or a light chain with three CDRs comprising the amino acid sequences SSNIGRNA (SEQ ID NO:835), SNN (SEQ ID NO:836), and SAWDTSLSTWV (SEQ ID NO:837) respectively;
m. a heavy chain with three CDRs comprising the amino acid sequences GFTFSSYW (SEQ ID NO:790), IKQDGSEK (SEQ ID NO:791), and ARGFYYYGAFDI (SEQ ID NO:792) respectively, and/or a light chain with three CDRs comprising the amino acid sequences SGSIASNY (SEQ ID NO:838), EDN (SEQ ID NO:839), and QSYDSSNHWV (SEQ ID NO:840) respectively;
n. a heavy chain with three CDRs comprising the amino acid sequences GFTFDDYA (SEQ ID NO:793), IDWNSGVI (SEQ ID NO:794), and AKDAYSYGFLGAFDI (SEQ ID NO:795) respectively, and/or a light chain with three CDRs comprising the amino acid sequences NIRTKG (SEQ ID NO:841), YAS (SEQ ID NO:842), and QVWDSSSDLVV (SEQ ID NO:843) respectively;
o. a heavy chain with three CDRs comprising the amino acid sequences GYSFTGSH (SEQ ID NO:796), INPDSGVI (SEQ ID NO:797), and ARDKAIGYVWALDY (SEQ ID NO:798) respectively, and/or a light chain with three CDRs comprising the amino acid sequences SSDVGTYNR (SEQ ID NO:844), EVS (SEQ ID NO:845), and SSYTRTFTYV (SEQ ID NO:846) respectively; or
p. a heavy chain with three CDRs comprising the amino acid sequences GASISNSF (SEQ ID NO:799), TSYSGNS (SEQ ID NO:800), and ARREWIKGHFDY (SEQ ID NO:801) respectively, and/or a light chain with three CDRs comprising the amino acid sequences GGSIASNY (SEQ ID NO:847), EDN (SEQ ID NO:848), and QSYDSSNPVV (SEQ ID NO:849) respectively.

30. The antibody of claim 1, wherein the antibody comprises:
a. a V_{H} amino acid sequence having SEQ ID NO: 722, and a V_{L} amino acid sequence having SEQ ID NO: 723;
b. a V_{H} amino acid sequence having SEQ ID NO: 724, and a V_{L} amino acid sequence having SEQ ID NO: 725;
c. a V_{H} amino acid sequence having SEQ ID NO: 726, and a V_{L} amino acid sequence having SEQ ID NO: 727;
d. a V_{H} amino acid sequence having SEQ ID NO: 728, and a V_{L} amino acid sequence having SEQ ID NO: 729;
e. a V_{H} amino acid sequence having SEQ ID NO: 730, and a V_{L} amino acid sequence having SEQ ID NO: 731;
f. a V_{H} amino acid sequence having SEQ ID NO: 732, and a V_{L} amino acid sequence having SEQ ID NO: 733;
g. a V_{H} amino acid sequence having SEQ ID NO: 734, and a V_{L} amino acid sequence having SEQ ID NO: 735;
h. a V_{H} amino acid sequence having SEQ ID NO: 736, and a V_{L} amino acid sequence having SEQ ID NO: 737;
i. a V_{H} amino acid sequence having SEQ ID NO: 738, and a V_{L} amino acid sequence having SEQ ID NO: 739;
j. a V_{H} amino acid sequence having SEQ ID NO: 740, and a V_{L} amino acid sequence having SEQ ID NO: 741;
k. a V_{H} amino acid sequence having SEQ ID NO: 742, and a V_{L} amino acid sequence having SEQ ID NO: 743;
l. a V_{H} amino acid sequence having SEQ ID NO: 744, and a V_{L} amino acid sequence having SEQ ID NO: 745;
m. a V_{H} amino acid sequence having SEQ ID NO: 746, and a V_{L} amino acid sequence having SEQ ID NO: 747;
n. a V_{H} amino acid sequence having SEQ ID NO: 748, and a V_{L} amino acid sequence having SEQ ID NO: 749;
o. a V_{H} amino acid sequence having SEQ ID NO: 750, and a V_{L} amino acid sequence having SEQ ID NO: 751; or
p. a V_{H} amino acid sequence having SEQ ID NO: 752, and a V_{L} amino acid sequence having SEQ ID NO: 753.

31. The antibody of claim 15, wherein the antibody comprises:
a. a heavy chain with three CDRs comprising the amino acid sequences GFSLSTSGVG (SEQ ID NO:754), IYWDDDK (SEQ ID NO:755), and ARISGSGYFYPFDI (SEQ ID NO:756) respectively, and/or a light chain with three CDRs comprising the amino acid sequences SGSIASNY (SEQ ID NO:802), EDN (SEQ ID NO:803), and QSYDSSNLWV (SEQ ID NO:804) respectively;
b. a heavy chain with three CDRs comprising the amino acid sequences GDSVSSNSAA (SEQ ID NO:757), TYYRSRWYN (SEQ ID NO:758), and AREIRGFDY (SEQ ID NO:759) respectively, and/or a light chain with three CDRs comprising the amino acid sequences SSDVGAYNF (SEQ ID NO:805), DFN (SEQ ID NO:806), and SSYAGSNNFDVV (SEQ ID NO:807) respectively;
c. a heavy chain with three CDRs comprising the amino acid sequences GFTFGDYA (SEQ ID NO:760), IRSKAYGGTT (SEQ ID NO:761), and TTADDDMDV (SEQ ID NO:762) respectively, and/or a light chain with three CDRs comprising the amino acid sequences SGTIASNY (SEQ ID NO:808), EDN (SEQ ID NO:809), and QSYDTSNHYV (SEQ ID NO:810) respectively;
d. a heavy chain with three CDRs comprising the amino acid sequences GFTFSNYG (SEQ ID NO:763), IWERGSKK (SEQ ID NO:764), and AREGISMTGAEYFQH (SEQ ID NO:765) respectively, and/or a light chain with three CDRs comprising the amino acid sequences SSNIGAGYD (SEQ ID NO:811), GTN (SEQ ID NO:812), and QSYDNSLTDPYV (SEQ ID NO:813) respectively;
e. a heavy chain with three CDRs comprising the amino acid sequences GFTFDDYA (SEQ ID NO:766), IDWNSGVI (SEQ ID NO:767), and AKDIGPGGSGSYYAFDI (SEQ ID NO:768) respectively, and/or a light chain with three CDRs comprising the amino acid sequences SSDVGGSKY (SEQ ID NO:814), DVT (SEQ ID NO:815), and AAWDDSLNGVV (SEQ ID NO:816) respectively;
f. a heavy chain with three CDRs comprising the amino acid sequences GFSFSRYG (SEQ ID NO:769), IRHDGSKK (SEQ ID NO:770), and AKDGRLEAALDD (SEQ ID NO:771) respectively, and/or a light chain with three CDRs comprising the amino acid sequences SGSIANNF (SEQ ID NO:817), EDN (SEQ ID NO:818), and QSYDSSNLV (SEQ ID NO:819) respectively;
g. a heavy chain with three CDRs comprising the amino acid sequences GYSFTSYW (SEQ ID NO:772), IYPGDSDT (SEQ ID NO:773), and ARRGDLDAFDI (SEQ ID NO:774) respectively, and/or a light chain with three CDRs comprising the amino acid sequences SANIGSNA (SEQ ID NO:820), GNT (SEQ ID NO:821), and AAWDDSLNGYV (SEQ ID NO:822) respectively;
h. a heavy chain with three CDRs comprising the amino acid sequences GYRLSDYY (SEQ ID NO:775), IKQDGSEK (SEQ ID NO:776), and ARVRGWSRGYFDY (SEQ ID NO:777) respectively, and/or a light chain with three CDRs comprising the amino acid sequences SGSIASNY (SEQ ID NO:823), EDN (SEQ ID NO:824), and QSYDSSNHWV (SEQ ID NO:825) respectively;
i. a heavy chain with three CDRs comprising the amino acid sequences GFTFDDYA (SEQ ID NO:778), ISWNSGSI (SEQ ID NO:779), and ARDWWGSIDH (SEQ ID NO:780) respectively, and/or a light chain with three CDRs comprising the amino acid sequences SSDVGGYDY (SEQ ID NO:826), DVS (SEQ ID NO:827), and SSYTSSSPVV (SEQ ID NO:828) respectively;
j. a heavy chain with three CDRs comprising the amino acid sequences GFTFSHYD (SEQ ID NO:781), IGYDGTNL (SEQ ID NO:782), and ARAANYYDSSGYGRADAFDI (SEQ ID NO:783) respectively, and/or a light chain with three CDRs comprising the amino acid sequences TGSIAGNY (SEQ ID NO:829), DDN (SEQ ID NO:830), and QSYDSGNRGV (SEQ ID NO:831) respectively;
k. a heavy chain with three CDRs comprising the amino acid sequences GGTFSTYG (SEQ ID NO:784), IIPSLGIP (SEQ ID NO:785), and ARENIDLATNDF (SEQ ID NO:786) respectively, and/or a light chain with three CDRs comprising the amino acid sequences SRDIGAYGY (SEQ ID NO:832), EVR (SEQ ID NO:833), and SSYTSSSTLDVV (SEQ ID NO:834) respectively;
l. a heavy chain with three CDRs comprising the amino acid sequences GGTFSSSG (SEQ ID NO:787), IIPMLGTP (SEQ ID NO:788), and ARDGGNYDY (SEQ ID NO:789) respectively, and/or a light chain with three CDRs comprising the amino acid sequences SSNIGRNA (SEQ ID NO:835), SNN (SEQ ID NO:836), and SAWDTSLSTWV (SEQ ID NO:837) respectively;
m. a heavy chain with three CDRs comprising the amino acid sequences GFTFSSYW (SEQ ID NO:790), IKQDGSEK (SEQ ID NO:791), and ARGFYYYGAFDI (SEQ ID NO:792) respectively, and/or a light chain with three CDRs comprising the amino acid sequences SGSIASNY (SEQ ID NO:838), EDN (SEQ ID NO:839), and QSYDSSNHWV (SEQ ID NO:840) respectively;
n. a heavy chain with three CDRs comprising the amino acid sequences GFTFDDYA (SEQ ID NO:793), IDWNSGVI (SEQ ID NO:794), and AKDAYSYGFLGAFDI (SEQ ID NO:795) respectively, and/or a light chain with three CDRs comprising the amino acid sequences NIRTKG (SEQ ID NO:841), YAS (SEQ ID NO:842), and QVWDSSSDLVV (SEQ ID NO:843) respectively;
o. a heavy chain with three CDRs comprising the amino acid sequences GYSFTGSH (SEQ ID NO:796), INPDSGVI (SEQ ID NO:797), and ARDKAIGYVWALDY (SEQ ID NO:798) respectively, and/or a light chain with three CDRs comprising the amino acid sequences SSDVGTYNR (SEQ ID NO:844), EVS (SEQ ID NO:845), and SSYTRTFTYV (SEQ ID NO:846) respectively; or
p. a heavy chain with three CDRs comprising the amino acid sequences GASISNSF (SEQ ID NO:799), TSYSGNS (SEQ ID NO:800), and ARREWIKGHFDY (SEQ ID NO:801) respectively, and/or a light chain with three CDRs comprising the amino acid sequences GGSIASNY (SEQ ID NO:847), EDN (SEQ ID NO:848), and QSYDSSNPVV (SEQ ID NO:849) respectively.

32. The antibody of claim 1, wherein the antibody comprises:
a. a V_{H} amino acid sequence having SEQ ID NO: 722, and a V_{L} amino acid sequence having SEQ ID NO: 723;
b. a V_{H} amino acid sequence having SEQ ID NO: 724, and a V_{L} amino acid sequence having SEQ ID NO: 725;
c. a V_{H} amino acid sequence having SEQ ID NO: 726, and a V_{L} amino acid sequence having SEQ ID NO: 727;
d. a V_{H} amino acid sequence having SEQ ID NO: 728, and a V_{L} amino acid sequence having SEQ ID NO: 729;
e. a V_{H} amino acid sequence having SEQ ID NO: 730, and a V_{L} amino acid sequence having SEQ ID NO: 731;
f. a V_{H} amino acid sequence having SEQ ID NO: 732, and a V_{L} amino acid sequence having SEQ ID NO: 733;
g. a V_{H} amino acid sequence having SEQ ID NO: 734, and a V_{L} amino acid sequence having SEQ ID NO: 735;
h. a V_{H} amino acid sequence having SEQ ID NO: 736, and a V_{L} amino acid sequence having SEQ ID NO: 737;
i. a V_{H} amino acid sequence having SEQ ID NO: 738, and a V_{L} amino acid sequence having SEQ ID NO: 739;
j. a V_{H} amino acid sequence having SEQ ID NO: 740, and a V_{L} amino acid sequence having SEQ ID NO: 741;
k. a V_{H} amino acid sequence having SEQ ID NO: 742, and a V_{L} amino acid sequence having SEQ ID NO: 743;
l. a V_{H} amino acid sequence having SEQ ID NO: 744, and a V_{L} amino acid sequence having SEQ ID NO: 745;
m. a V_{H} amino acid sequence having SEQ ID NO: 746, and a V_{L} amino acid sequence having SEQ ID NO: 747;
n. a V_{H} amino acid sequence having SEQ ID NO: 748, and a V_{L} amino acid sequence having SEQ ID NO: 749;
o. a V_{H} amino acid sequence having SEQ ID NO: 750, and a V_{L} amino acid sequence having SEQ ID NO: 751; or
p. a V_{H} amino acid sequence having SEQ ID NO: 752, and a V_{L} amino acid sequence having SEQ ID NO: 753.

33. A method of preventing a disease or disorder caused by Severe Acute Respiratory Syndrome coronavirus (SARS-CoV2), the method comprising administering to a subject at risk of suffering from the disease or disorder, a therapeutically effective amount of the multispecific antibody of claim 1 or the scFv antibody of claim 15.

34. The method of claim 33, wherein the method further comprises administering an anti-viral drug, a viral entry inhibitor, a viral attachment inhibitor, or a combination thereof.

35. The method of claim 33, wherein the method comprises administering two or more antibodies specific to SARS-CoV2.

36. The method of claim 33, wherein the antibody is administered prior to or after exposure to SARS-CoV2.

37. The method of claim 33, wherein the antibody is administered at a dose sufficient to neutralize the SARS-CoV2.

38.-51. (canceled)
